(19) United States
(12) Patent Application Publication
(10) Pub. No.: US 2023/0399329 A1
(43) Pub. Date: Dec. 14, 2023

(54) HETEROCYCLIC COMPOUND, ORGANIC LIGHT-EMITTING DEVICE COMPRISING SAME, AND COMPOSITION FOR FORMING ORGANIC LAYER

(71) Applicant: LT MATERIALS CO., LTD., Yongin-si, Gyeonggi-do (KR)

(72) Inventors: Yu Jin HEO, Yongin-si (KR); Won Jang JEONG, Yongin-si (KR); Gi Back LEE, Yongin-si (KR); Dong Jun KIM, Yongin-si (KR)

(73) Assignee: LT MATERIALS CO., LTD., Yongin-si, Gyeonggi-do (KR)

(21) Appl. No.: 18/036,136

(22) PCT Filed: Nov. 12, 2021
(86) PCT No.: PCT/KR2021/016553

§ 371 Date: May 9, 2023

(30) Foreign Application Priority Data
Nov. 17, 2020 (KR) 10-2020-0153293

(51) Int. Cl. C07D 471/04; C07D 209/82; C07D 401/14; H10K 50/16; H10K 85/60; C07D 417/14; C07D 231/56; C07D 413/14; C07F 9/28

(52) U.S. Cl. CPC C07D 471/04; C07D 209/82; C07D 401/14; H10K 50/16; H10K 85/6576; C07D 417/14; C07D 231/56; C07D 413/14; H10K 85/6574; H10K 85/615; H10K 85/654; C07F 9/28

(57) ABSTRACT

The present invention provides a heterocyclic compound represented by Formula 1, an organic light-emitting device comprising the same, and a composition for forming an organic layer.

### TECHNICAL FIELD

This application claims the benefit of priority based on Korean Patent Application No. 10-2020-0153293 filed on Nov. 17, 2020, and the entire contents disclosed in the literatures of said Korean Patent Application are incorporated as part of the present specification.

The present invention relates to a heterocyclic compound, an organic light-emitting device comprising the same, and a composition for forming an organic layer.

### BACKGROUND ART

An organic light-emitting device (organic light-emitting diode; OLED) has recently received a lot of attention due to an increase in demand for flat panel display devices. The organic light-emitting device is a device that converts electrical energy into light, and the performance of the organic light-emitting device is greatly affected by an organic material positioned between electrodes.

The organic light-emitting device has a structure in which an organic thin film is disposed between two electrodes. When a voltage is applied to the organic light-emitting device having such a structure, electrons and holes injected from the two electrodes combine in the organic thin film to form a pair, and then emit light while disappearing. The organic thin film may be composed of a single layer or multiple layers, if necessary.

The organic thin film material may have a light-emitting function, if necessary. For example, as the organic thin film material, a compound capable of constituting the light-emitting layer by itself may be used, or a compound capable of serving as a host or dopant of the host-dopant-based light-emitting layer may be used. In addition, as the organic thin film material, a compound capable of serving as a hole injection layer, a hole transport layer, an electron-blocking layer, a hole-blocking layer, an electron transport layer, an electron injection layer, an electron-generating layer, and the like may be used.

In order to improve the performance, lifespan, or efficiency of the organic light-emitting device, the development of the organic thin film material is continuously required.

### PRIOR ART REFERENCES

### Patent Documents

Korean Patent No. 10-1838693

### DISCLOSURE

### Technical Problem

It is an object of the present invention to provide a heterocyclic compound capable of imparting a low driving voltage, excellent luminous efficiency, and excellent lifespan properties to an organic light-emitting device.

It is another object of the present invention to provide an organic light-emitting device comprising the heterocyclic compound.

It is another object of the present invention to provide a composition for forming an organic layer comprising the heterocyclic compound.

### Technical Solution

The present invention provides a heterocyclic compound represented by following Formula 1:

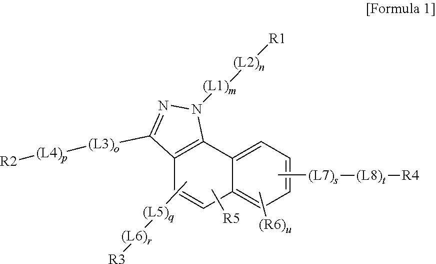

wherein:
R1 to R4 are the same as or different from each other and are each independently hydrogen, deuterium, halogen, a cyano group, a substituted or unsubstituted C1 to C60 alkyl group, a substituted or unsubstituted C2 to C60 alkenyl group, a substituted or unsubstituted C2 to C60 alkynyl group, a substituted or unsubstituted C1 to C60 alkoxy group, a substituted or unsubstituted C3 to C60 cycloalkyl group, a substituted or unsubstituted C2 to C60 heterocycloalkyl group, a substituted or unsubstituted C6 to C60 aryl group, a substituted or unsubstituted C2 to C60 heteroaryl group, or —P(═O)R101R102R103, wherein R101, R102, and R103 are the same as or different from each other and are each independently a substituted or unsubstituted C6 to C60 aryl group or a substituted or unsubstituted C2 to C60 heteroaryl group;
R5 and R6 are the same as or different from each other and are each independently hydrogen, deuterium, halogen, a cyano group, a substituted or unsubstituted C1 to C60 alkyl group, a substituted or unsubstituted C2 to C60 alkenyl group, a substituted or unsubstituted C2 to C60 alkynyl group, a substituted or unsubstituted C1 to C60 alkoxy group, a substituted or unsubstituted C3 to C60 cycloalkyl group, a substituted or unsubstituted C2 to C60 heterocycloalkyl group, a substituted or unsubstituted C6 to C60 aryl group, or a substituted or unsubstituted C2 to C60 heteroaryl group;
L1 to L8 are the same as or different from each other and are each independently a direct bond, a substituted or unsubstituted C6 to C60 arylene group, or a substituted or unsubstituted C2 to C60 heteroarylene group;
m, n, o, p, q, r, s, and t are the same as or different from each other and are each independently an integer of 0 to 2, provided that when m, n, o, p, q, r, s, and t are 2, each L1 to L8 defined by these are the same as or different from each other and are each independently selected; and
u is an integer of 0 to 4, provided that when u is 2 to 4, R6 is the same as or different from each other and is each independently selected.

In addition, the present invention provides an organic light-emitting device comprising:
a first electrode;
a second electrode provided to face the first electrode; and
one or more organic layers provided between the first electrode and the second electrode, and
wherein one or more of the organic layers comprise the heterocyclic compound represented by Formula 1.

In addition, the present invention provides a composition for forming an organic layer of an organic light-emitting device, comprising the heterocyclic compound represented by Formula 1.

### Advantageous Effects

The heterocyclic compound of the present invention and the composition for an organic layer comprising the same may be usefully used as a material for an organic layer of an organic light-emitting device. In particular, these are used as materials for an electron transport layer, a charge-generating layer, an electron injection layer, an electron-blocking layer, and a hole-blocking layer, thereby providing remarkable effects of lowering the driving voltage of the organic light-emitting device, improving the luminous efficiency, and improving the lifespan properties.

The organic light-emitting device of the present invention comprises the heterocyclic compound or the composition for an organic layer comprising the same, thereby providing excellent driving voltage, luminous efficiency, and lifespan properties.

### DESCRIPTION OF DRAWINGS

FIGS. 1 to 4 are schematic views showing a stacked structure of an organic light-emitting device according to one embodiment of the present invention, respectively.

### BEST MODE

Hereinafter, the present invention will be described in detail.

In the present invention, the term “substituted” means that a hydrogen atom bonded to a carbon atom of a compound is replaced with another substituent, and the position to be substituted is not limited as long as it is the position at which a hydrogen atom is substituted, that is, the position at which the substituent is substitutable. When two or more substituents are substituted, the two or more substituents may be the same as or different from each other.

In the present invention, the term “substituted or unsubstituted” means that it is unsubstituted or substituted by one or more substituents selected from the group insisting of C1 to C60 linear or branched alkyl, C2 to C60 linear or branched alkenyl, C2 to C60 linear or branched alkynyl, C3 to C60 monocyclic or polycyclic cycloalkyl, C2 to C60 monocyclic or polycyclic heterocycloalkyl, C6 to C60 monocyclic or polycyclic aryl, C2 to C60 monocyclic or polycyclic heteroaryl, —SiRR′R″, —P(═O)RR′, C1 to C20 alkylamine, C6 to C60 monocyclic or polycyclic arylamine, and C2 to C60 monocyclic or polycyclic heteroarylamine; or it is unsubstituted or substituted by a substituent to which two or more substituents selected from the above-exemplified substituents are connected.

In the present invention, the alkyl group includes a linear or branched chain having 1 to 60 carbon atoms, and may be further substituted by another substituent. The number of carbon atoms in the alkyl group may be 1 to 60, specifically 1 to 40, more specifically 1 to 20. Specific examples include, but are not limited to, methyl group, ethyl group, propyl group, n-propyl group, isopropyl group, butyl group, n-butyl group, isobutyl group, tert-butyl group, sec-butyl group, 1-methyl-butyl group, 1-ethylbutyl group, pentyl group, n-pentyl group, isopentyl group, neopentyl group, tert-pentyl group, hexyl group, n-hexyl group, 1-methylpentyl group, 2-methylpentyl group, 4-methyl-2-pentyl group, 3,3-dimethylbutyl group, 2-ethylbutyl group, heptyl group, n-heptyl group, 1-methylhexyl group, cyclopentylmethyl group, cyclohexylmethyl group, octyl group, n-octyl group, tert-octyl group, 1-methylheptyl group, 2-ethylhexyl group, 2-propylpentyl group, n-nonyl group, 2,2-dimethylheptyl group, 1-ethyl-propyl group, 1,1-dimethyl-propyl group, isohexyl group, 2-methylpentyl group, 4-methylhexyl group, 5-methylhexyl group, and the like.

In the present invention, the alkenyl group includes a linear or branched chain having 2 to 60 carbon atoms, and may be further substituted by another substituent. The number of carbon atoms in the alkenyl group may be 2 to 60, specifically 2 to 40, more specifically 2 to 20. Specific examples include, but are not limited to, a vinyl group, 1-propenyl group, isopropenyl group, 1-butenyl group, 2-butenyl group, 3-butenyl group, 1-pentenyl group, 2-pentenyl group, 3-pentenyl group, 3-methyl-1-butenyl group, 1,3-butadienyl group, allyl group, 1-phenylvinyl-1-yl group, 2-phenylvinyl-1-yl group, 2,2-diphenylvinyl-1-yl group, 2-phenyl-2-(naphthyl-1-yl)vinyl-1-yl group, 2,2-bis(diphenyl-1-yl)vinyl-1-yl group, stilbenyl group, styrenyl group, and the like.

In the present invention, the alkynyl group includes a linear or branched chain having 2 to 60 carbon atoms, and may be further substituted by another substituent. The number of carbon atoms in the alkynyl group may be 2 to 60, specifically 2 to 40, more specifically 2 to 20.

In the present invention, the cycloalkyl group includes a monocyclic or polycyclic ring having 3 to 60 carbon atoms, and may be further substituted by another substituent. Herein, the polycyclic refers to a group in which a cycloalkyl group is directly connected or condensed with another cyclic group. Herein, the another cyclic group may be a cycloalkyl group, but may be a different type of cyclic group, for example, a heterocycloalkyl group, an aryl group, a heteroaryl group, and the like. The number of carbon atoms in the cycloalkyl group may be 3 to 60, specifically 3 to 40, more specifically 5 to 20. Specific examples include, but are not limited to, cyclopropyl group, cyclobutyl group, cyclopentyl group, 3-methylcyclopentyl group, 2,3-dimethylcyclopentyl group, cyclohexyl group, 3-methylcyclohexyl group, 4-methylcyclohexyl group, 2,3-dimethylcyclohexyl group, 3,4,5-trimethylcyclohexyl group, 4-tert-butylcyclohexyl group, cycloheptyl group, cyclooctyl group, and the like.

In the present invention, the heterocycloalkyl group includes O, S, Se, N, or Si as a heteroatom, includes a monocyclic or polycyclic ring having 2 to 60 carbon atoms, and may be further substituted by another substituent. Herein, polycyclic refers to a group in which a heterocycloalkyl group is directly connected or condensed with another cyclic group. Herein, another cyclic group may be a heterocycloalkyl group, but may be a different type of cyclic group, for example, a cloalkyl group, an aryl group, a heteroaryl group, and the like. The number of carbon atoms in the heterocycloalkyl group may be 2 to 60, specifically 2 to 40, more specifically 3 to 20.

In the present invention, the aryl group includes a monocyclic or polycyclic ring having 6 to 60 carbon atoms, and may be further substituted by other substituents. Herein, the polycyclic refers to a group in which an aryl group is directly connected or condensed with another cyclic group. Herein, the another cyclic group may be an aryl group, but may be a different type of cyclic group, for example, a cloalkyl group, a heterocycloalkyl group, a heteroaryl group, and the like. The aryl group includes a spiro group. The number of carbon atoms in the aryl group may be 6 to 60, specifically 6 to 40, more specifically 6 to 25. Specific examples of the aryl group may include, but are not limited to, a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, tetracenyl group, pentacenyl group, fluorenyl group, indenyl group, acenaphthylenyl group, benzofluorenyl group, spirobifluorenyl group, 2,3-dihydro-1H-indenyl group, condensed cyclic groups thereof, and the like.

In the present invention, the fluorenyl group may be substituted, and adjacent substituents may be bonded to each other to form a ring.

When the fluorenyl group is substituted, it may be, but is not limited to,

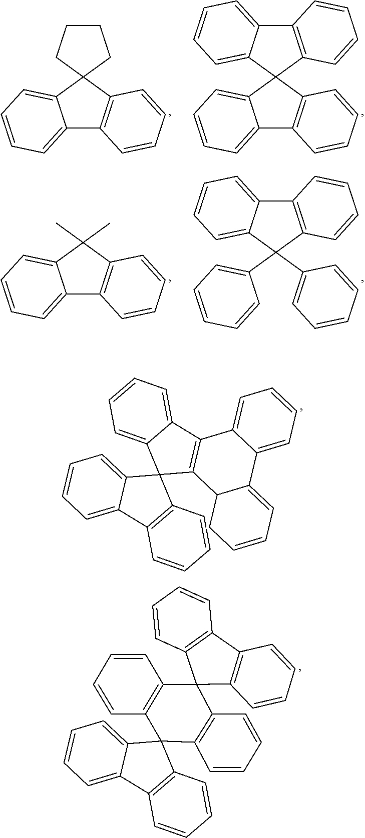

and the like.

In the present invention, the heteroaryl group includes S, O, Se, N, or Si as a heteroatom, includes a monocyclic or polycyclic ring having 2 to 60 carbon atoms, and may be further substituted by other substituents. Herein, the polycyclic refers to a group in which a heteroaryl group is directly connected or condensed with another cyclic group. Herein, the another cyclic group may be a heteroaryl group, but may be a different type of cyclic group, for example, a cloalkyl group, a heterocycloalkyl group, an aryl group, and the like. The number of carbon atoms in the heteroaryl group may be 2 to 60, specifically 2 to 40, more specifically 3 to 25. Specific examples of the heteroaryl group may include, but are not limited to, a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a triazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a triazinyl group, a dioxynyl group, a triazinyl group, a tetrazinyl group, a quinolyl group, an isoquinolyl group, a quinazolinyl group, an isoquinazolinyl group, a quinozolylyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilol group, a spirobi(dibenzosilole) group, a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like.

In the present invention, the amine group may be selected from the group consisting of a monoalkylamine group, monoarylamine group, monoheteroarylamine group, —NH₂, a dialkylamine group, a diarylamine group, a diheteroarylamine group, an alkylarylamine group, an alkylheteroarylamine group, and an arylheteroarylamine group; and the number of carbon atoms is not particularly limited, but is preferably 1 to 30. Specific examples of the amine group include, but are not limited to, a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like.

In the present invention, the arylene group refers to a group having two bonding positions on the aryl group, that is, a divalent group. The description of the aryl group described above may be applied, except that each of these is a divalent group. In addition, the heteroarylene group refers to a group having two bonding positions on the heteroaryl group, that is, a divalent group. The description of the heteroaryl group described above may be applied, except that each of these is a divalent group.

In the present invention, an “adjacent” group may refer to a substituent substituted on an atom directly connected to the atom on which a certain substituent is substituted, a substituent sterically closest to a certain substituent, or another substituent substituted on the atom in which a certain substituent is substituted. For example, two substituents substituted at an ortho position on a benzene ring and two substituents substituted at the same carbon on an aliphatic ring may be interpreted as “adjacent” groups to each other.

In the present invention, “the case where a substituent is not indicated in the chemical formula or compound structure” means that a hydrogen atom is bonded to a carbon atom. However, since deuterium (²H) is an isotope of hydrogen, some hydrogen atoms may be deuterium.

In one embodiment of the present invention, “the case where a substituent is not indicated in the chemical formula or compound structure” may mean that hydrogen or deuterium is present at all positions that may be substituted with a substituent. That is, since deuterium is an isotope of hydrogen, some hydrogen atoms may be isotope deuterium and the content of deuterium may be 0% to 100%.

In one embodiment of the present invention, in “the case where a substituent is not indicated in the chemical formula or compound structure,” hydrogen and deuterium may be used interchangeably in compounds unless deuterium is explicitly excluded, such as “the content of deuterium is 0%,” “the content of hydrogen is 100%,” and “all substituents are hydrogen.”

In one embodiment of the present invention, deuterium is one of the isotopes of hydrogen and is an element having a deuteron consisting of one proton and one neutron as a nucleus, and may be expressed as hydrogen-2, and its element symbol may also be written as D or 2H.

In one embodiment of the present invention, isotopes, which refer to atoms having the same atomic number (Z) but different mass numbers (A), may also be interpreted as elements having the same number of protons but a different number of neutrons.

In one embodiment of the present invention, the meaning of the T % content of a specific substituent may be defined as the following formula: T2/T1×100=T %, wherein T1 is defined as the total number of substituents that the basic compound can have and T2 is defined as the number of a specific substituent.

That is, in one example, the 20% content of deuterium in the phenyl group represented by

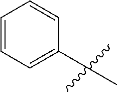 [SMILES: *c1ccccc1]

may mean that the total number of substituents that the phenyl group can have is 5 (T1 in the formula) and the number of deuterium is 1 (T2 in the formula). That is, the 20% content of deuterium in the phenyl group may be represented by the following structural formula:

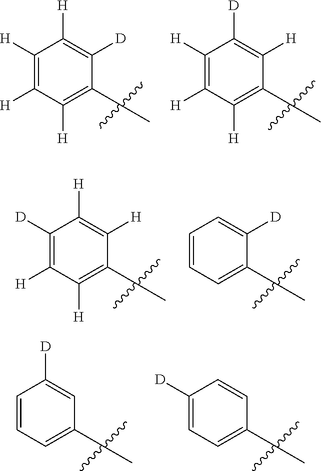

In addition, in one embodiment of the present invention, the case of “a phenyl group having a deuterium content of 0%” may mean a phenyl group that does not contain deuterium atoms, that is, has 5 hydrogen atoms.

In the present invention, the content of deuterium in the heterocyclic compound represented by Formula 1 may be 0 to 100%, more preferably 10 to 50%.

The present invention provides a heterocyclic compound represented by following Formula 1:

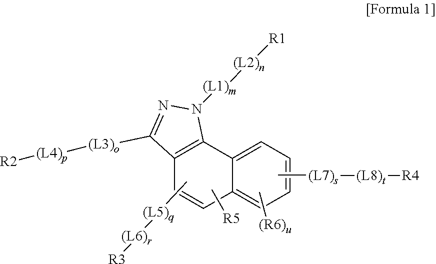

wherein:
R1 to R4 are the same as or different from each other and are each independently hydrogen, deuterium, halogen, a cyano group, a substituted or unsubstituted C1 to C60 alkyl group, a substituted or unsubstituted C2 to C60 alkenyl group, a substituted or unsubstituted C2 to C60 alkynyl group, a substituted or unsubstituted C1 to C60 alkoxy group, a substituted or unsubstituted C3 to C60 cycloalkyl group, a substituted or unsubstituted C2 to C60 heterocycloalkyl group, a substituted or unsubstituted C6 to C60 aryl group, a substituted or unsubstituted C2 to C60 heteroaryl group, or —P(═O)R101R102R103, wherein R101, R102, and R103 are the same as or different from each other and are each independently a substituted or unsubstituted C6 to C60 aryl group or a substituted or unsubstituted C2 to C60 heteroaryl group;
R5 and R6 are the same as or different from each other and are each independently hydrogen, deuterium, halogen, a cyano group, a substituted or unsubstituted C1 to C60 alkyl group, a substituted or unsubstituted C2 to C60 alkenyl group, a substituted or unsubstituted C2 to C60 alkynyl group, a substituted or unsubstituted C1 to C60 alkoxy group, a substituted or unsubstituted C3 to C60 cycloalkyl group, a substituted or unsubstituted C2 to C60 heterocycloalkyl group, a substituted or unsubstituted C6 to C60 aryl group, or a substituted or unsubstituted C2 to C60 heteroaryl group;
L1 to L8 are the same as or different from each other and are each independently a direct bond, a substituted or unsubstituted C6 to C60 arylene group, or a substituted or unsubstituted C2 to C60 heteroarylene group;
m, n, o, p, q, r, s, and t are the same as or different from each other and are each independently an integer of 0 to 2, provided that when m, n, o, p, q, r, s, and t are 2, each L1 to L8 defined by these are the same as or different from each other and are each independently selected; and
u is an integer of 0 to 4, provided that when u is 2 to 4, R6 is the same as or different from each other and may be each independently selected.

In one embodiment of the present invention, the heteroatom in the heteroatom-containing group may be one or more selected from O, S, Se, N, or Si.

In another embodiment of the present invention, the heteroatom in the heteroatom-containing group may be one or more selected from O, S, or N.

In another embodiment of the present invention, the heteroatom in heteroatom-containing group may be N.

In one embodiment of the present invention, R1 to R4 may be the same as or different from each other and may be each independently hydrogen, deuterium, halogen, a cyano group, a substituted or unsubstituted C1 to C30 alkyl group, a substituted or unsubstituted C1 to C30 alkoxy group, a substituted or unsubstituted C6 to C60 aryl group, a substituted or unsubstituted C2 to C60 heteroaryl group, or —P(═O)R101R102R103, wherein R101, R102, and R103 may be the same as or different from each other and may be each independently a substituted or unsubstituted C6 to C60 aryl group or a substituted or unsubstituted C2 to C60 heteroaryl group.

In another embodiment of the present invention, R1 to R4 may be the same as or different from each other and may be each independently hydrogen, deuterium, halogen, a cyano group, a substituted or unsubstituted C1 to C20 alkyl group, a substituted or unsubstituted C6 to C30 aryl group, a substituted or unsubstituted C2 to C30 heteroaryl group, or —P(═O)R101R102R103, wherein R101, R102, and R103 may be the same as or different from each other and may be each independently a substituted or unsubstituted C6 to C30 aryl group or a substituted or unsubstituted C2 to C30 heteroaryl group.

In another embodiment of the present invention, R1 to R4 may be the same as or different from each other and may be each independently hydrogen, deuterium, a substituted or unsubstituted C1 to C10 alkyl group, a substituted or unsubstituted C6 to C20 aryl group, a substituted or unsubstituted C2 to C20 heteroaryl group, or —P(═O)R101R102R103, wherein R101, R102, and R103 may be the same as or different from each other and may be each independently a substituted or unsubstituted C6 to C20 aryl group or a substituted or unsubstituted C2 to C20 heteroaryl group.

In another embodiment of the present invention, R1 to R4 may be the same as or different from each other and may be each independently hydrogen, deuterium, substituted or unsubstituted phenyl, naphthalenyl, anthracenyl, phenanthrenyl,

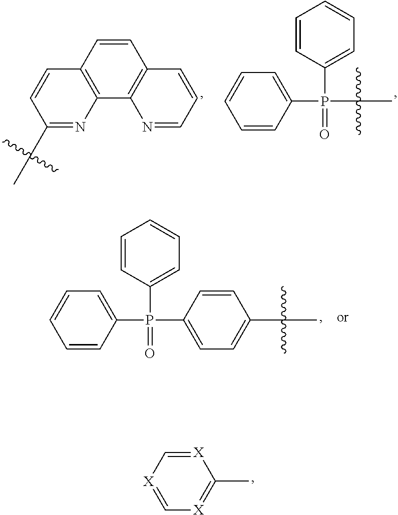

wherein X may be the same as or different from each other and may be a nitrogen atom or a carbon atom, provided that at least one may be a nitrogen atom.
Wherein two of X may be a nitrogen atom.
Wherein all of X may be a nitrogen atom.

In another embodiment of the present invention, three or more of R1 to R4 may be the same as or different from each other and may be each independently a substituted or unsubstituted C6 to C20 aryl group, a substituted or unsubstituted C2 to C20 heteroaryl group, or —P(═O)R101R102R103, wherein R101, R102, and R103 may be the same as or different from each other and may be each independently a substituted or unsubstituted C6 to C20 aryl group or a substituted or unsubstituted C2 to C20 heteroaryl group, and the other one may be hydrogen or deuterium.

In another embodiment of the present invention, R4 may be hydrogen or deuterium.

In one embodiment of the present invention, the substitution of R1 to R4 may be each independently made with one or more substituents selected from the group consisting of C1 to C10 linear or branched alkyl, C2 to C10 linear or branched alkenyl, C2 to C10 linear or branched alkynyl, C3 to C15 cycloalkyl, C2 to C20 heterocycloalkyl, C6 to C30 aryl, C2 to C30 heteroaryl, C1 to C10 alkylamine, C6 to C30 arylamine, and C2 to C30 heteroarylamine.

In another embodiment of the present invention, the substitution of R1 to R4 may be each independently made with one or more substituents selected from the group consisting of C1 to C10 linear or branched alkyl, C2 to C10 linear or branched alkenyl, C2 to C10 linear or branched alkynyl, C6 to C30 aryl, C2 to C30 heteroaryl, C6 to C30 arylamine, and C2 to C30 heteroarylamine.

In another embodiment of the present invention, the substitution of R1 to R4 may be each independently made with one or more substituents selected from the group consisting of C6 to C30 aryl, C2 to C30 heteroaryl, C6 to C30 arylamine, and C2 to C30 heteroarylamine.

In another embodiment of the present invention, the substitution of R1 to R4 may be each independently made with one or more substituents selected from the group consisting of phenyl, naphthalenyl, pyridinyl, anthracenyl, carbazole, biphenyl, dibenzothiophene, dibenzofuran, and phenanthrenyl.

In one embodiment of the present invention, R5 and R6 may be the same as or different from each other and may be each independently hydrogen, deuterium, a substituted or unsubstituted C6 to C30 aryl group, or a substituted or unsubstituted C2 to C30 heteroaryl group.

In another embodiment of the present invention, R5 and R6 may be the same as or different from each other and may be each independently hydrogen, deuterium, a substituted or unsubstituted C6 to C20 aryl group, or a substituted or unsubstituted C2 to C20 heteroaryl group.

In another embodiment of the present invention, R5 and R6 may be the same as or different from each other and may be each independently hydrogen, deuterium, substituted or unsubstituted phenyl, naphthalenyl, anthracenyl, phenanthrenyl,

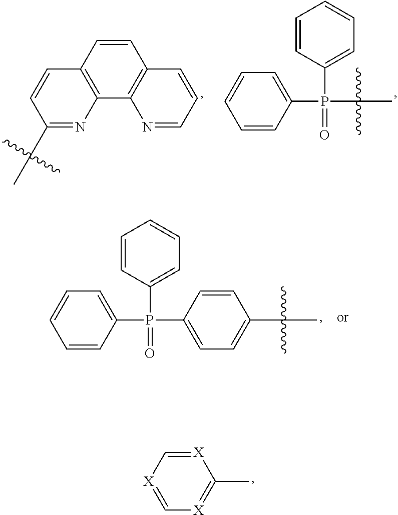

wherein X may be the same as or different from each other and may be each independently a nitrogen atom or a carbon atom, provided that at least one may be a nitrogen atom.
Wherein two of X may be a nitrogen atom.
Wherein all of X may be a nitrogen atom.

In one embodiment of the present invention, the substitution of R5 and R6 may be each independently made with one or more substituents selected from the group consisting of C1 to C10 linear or branched alkyl, C2 to C10 linear or branched alkenyl, C2 to C10 linear or branched alkynyl, C3 to C15 cycloalkyl, C2 to C20 heterocycloalkyl, C6 to C30 aryl, C2 to C30 heteroaryl, C1 to C10 alkylamine, C6 to C30 arylamine, and C2 to C30 heteroarylamine.

In another embodiment of the present invention, the substitution of R5 and R6 may be each independently made with one or more substituents selected from the group consisting of C1 to C10 linear or branched alkyl, C2 to C10 linear or branched alkenyl, C2 to C10 linear or branched alkynyl, C6 to C30 aryl, C2 to C30 heteroaryl, C6 to C30 arylamine, and C2 to C30 heteroarylamine.

In another embodiment of the present invention, the substitution of R5 and R6 may be each independently made with one or more substituents selected from the group consisting of C6 to C30 aryl, C2 to C30 heteroaryl, C6 to C30 arylamine, and C2 to C30 heteroarylamine.

In another embodiment of the present invention, the substitution of R5 and R6 may be each independently made with one or more substituents selected from the group consisting of phenyl, naphthalenyl, pyridinyl, anthracenyl, carbazole, biphenyl, dibenzothiophene, dibenzofuran, and phenanthrenyl.

In one embodiment of the present invention, L1 to L8 may be the same as or different from each other and may be each independently a direct bond, a substituted or unsubstituted C6 to C30 arylene group, or a substituted or unsubstituted C2 to C30 heteroarylene group.

In another embodiment of the present invention, L1 to L8 may be the same as or different from each other and may be each independently a direct bond, a substituted or unsubstituted C6 to C20 arylene group, or a substituted or unsubstituted C2 to C20 heteroarylene group.

In another embodiment of the present invention, L1 to L8 may be the same as or different from each other and may be each independently a direct bond, substituted or unsubstituted

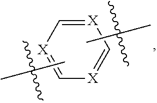

phenylene, naphthalene, anthracene, phenanthrene, or

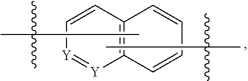

wherein X may be the same as or different from each other and may be each independently a nitrogen atom or a carbon atom, provided that at least one may be a nitrogen atom; and Y may be the same as or different from each other and may be each independently a nitrogen atom or a carbon atom, provided that at least one may be a nitrogen atom.
Wherein two of X may be a nitrogen atom.
Wherein all of X may be a nitrogen atom.
Wherein all of Y may be a nitrogen atom.

In another embodiment of the present invention, the substitution of L1 to L8 may be each independently made with one or more substituents selected from the group consisting of C1 to C10 linear or branched alkyl, C2 to C10 linear or branched alkenyl, C2 to C10 linear or branched alkynyl, C3 to C15 cycloalkyl, C2 to C20 heterocycloalkyl, C6 to C30 aryl, C2 to C30 heteroaryl, C1 to C10 alkylamine, C6 to C30 arylamine, and C2 to C30 heteroarylamine.

In another embodiment of the present invention, the substitution of L1 to L8 may be each independently made with one or more substituents selected from the group consisting of C1 to C10 linear or branched alkyl, C2 to C10 linear or branched alkenyl, C2 to C10 linear or branched alkynyl, C6 to C30 aryl, C2 to C30 heteroaryl, C6 to C30 arylamine, and C2 to C30 heteroarylamine.

In another embodiment of the present invention, the substitution of L1 to L8 may be each independently made with one or more substituents selected from the group consisting of C6 to C30 aryl, C2 to C30 heteroaryl, C6 to C30 arylamine, and C2 to C30 heteroarylamine.

In another embodiment of the present invention, the substitution of L1 to L8 may be each independently made with one or more substituents selected from the group consisting of phenyl, naphthalenyl, pyridinyl, anthracenyl, carbazole, biphenyl, dibenzothiophene, dibenzofuran, and phenanthrenyl.

In Formula 1 above, R1 to R3 may be the same as or different from each other and may be each independently a substituted or unsubstituted C6 to C60 aryl group, a substituted or unsubstituted C2 to C60 heteroaryl group, or —P(═O)R101R102R103, wherein R101, R102, and R103 may be the same as or different from each other and may be each independently a substituted or unsubstituted C6 to C60 aryl group or a substituted or unsubstituted C2 to C60 heteroaryl group.

In Formula 1 above, more preferably, any one or more of -(L1)m-(L2)n-R1, -(L3)o-(L4)p-R2, -(L5)q-(L6)r-R3, and -(L7)s-(L8)t-R4 may comprise 2 to 8 aromatic rings with or without a heteroatom, wherein the 2 to 8 aromatic rings may be composed of a monocyclic aromatic ring, an aromatic ring contained in a polycyclic condensed aromatic ring, or an aromatic ring included in a monocyclic aromatic ring and a polycyclic condensed aromatic ring.

In addition, wherein any one or more of -(L1)m-(L2)n-R1, -(L3)o-(L4)p-R2, -(L5)q-(L6)r-R3, and -(L7)s-(L8)t-R4 may comprise 3 to 8 aromatic rings with or without a heteroatom, wherein the 3 to 8 aromatic rings with or without a heteroatom may be composed of a monocyclic aromatic ring, an aromatic ring contained in a polycyclic condensed aromatic ring, or an aromatic ring included in a monocyclic aromatic ring and a polycyclic condensed aromatic ring.

The monocyclic aromatic ring may be phenyl or

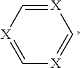

and the polycyclic condensed aromatic ring may be naphthalene, anthracene, phenanthrene, carbazole, dibenzothiophene, dibenzofuran,

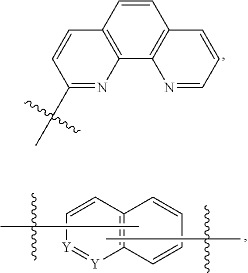

or the like, wherein X may be the same as or different from each other and may be each independently a nitrogen atom or a carbon atom, provided that at least one may be a nitrogen atom; and Y may be the same as or different from each other and may be each independently a nitrogen atom or a carbon atom, provided that at least one may be a nitrogen atom.

In Formula 1 above, much more preferably, R1 to R3 are the same as or different from each other and are each independently substituted or unsubstituted phenyl, naphthalenyl, anthracenyl, phenanthrenyl,

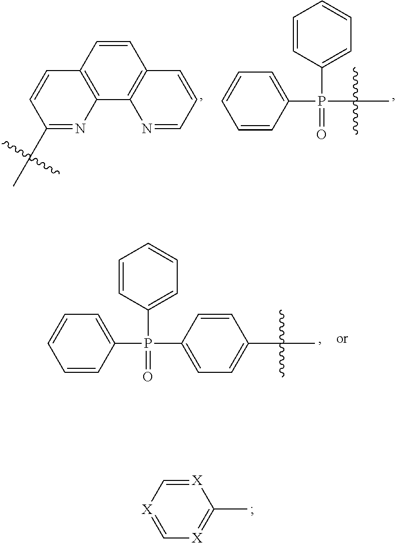

R4 is the same as or different from each other and is each independently hydrogen, deuterium, substituted or unsubstituted phenyl, naphthalenyl, anthracenyl, phenanthrenyl,

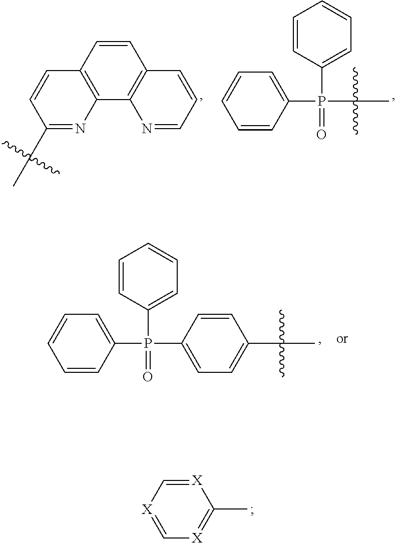

R5 and R6 are the same as or different from each other and are each independently hydrogen, deuterium, a substituted or unsubstituted C6 to C30 aryl group, or a substituted or unsubstituted C2 to C30 heteroaryl group;
L1 and L8 are the same as or different from each other and are each independently a direct bond, substituted or unsubstituted phenylene, naphthalene,

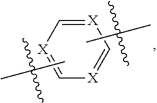

anthracene, phenanthrene, or

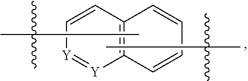

wherein X is the same as or different from each other and is each independently a nitrogen atom or a carbon atom, provided that at least one is a nitrogen atom; and Y is the same as or different from each other and is each independently a nitrogen atom or a carbon atom, provided that at least one is a nitrogen atom; and
wherein the substitution may be made with one or more substituents selected from the group consisting of substituted or unsubstituted phenyl, naphthalenyl, pyridinyl, anthracenyl, carbazole, biphenyl, dibenzothiophene, dibenzofuran, and phenanthrenyl.

In Formula 1 above, even more preferably, the heterocyclic compound represented by Formula 1 may be

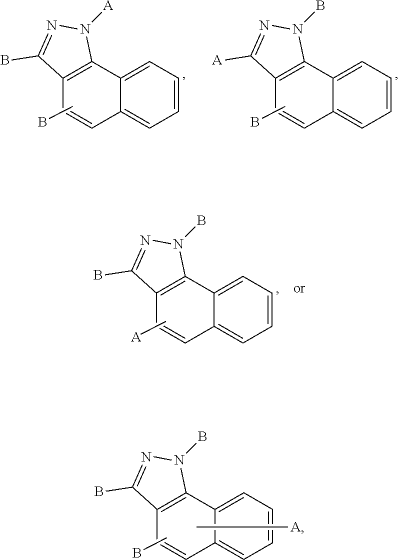

wherein:
A may comprise 2 to 8 aromatic rings with or without a heteroatom, wherein the 2 to 8 aromatic rings may be composed of a monocyclic aromatic ring, an aromatic ring contained in a polycyclic condensed aromatic ring, or an aromatic ring included in a monocyclic aromatic ring and a polycyclic condensed aromatic ring; and
B may be a substituted or unsubstituted C6 to C30 aryl group or a substituted or unsubstituted C2 to C30 heteroaryl group.
Wherein the monocyclic aromatic ring and the polycyclic aromatic ring are the same as described above.
In Formula 1 above, particularly preferably, A may be 5 to 7 aromatic rings with or without a heteroatom.
In Formula 1 above, particularly preferably, A may be 5 to 7 aromatic rings containing a heteroatom.
B may be a substituted or unsubstituted phenyl group.
In one embodiment of the present invention, the heterocyclic compound represented by Formula 1 may be a compound represented by any one of the following compounds:

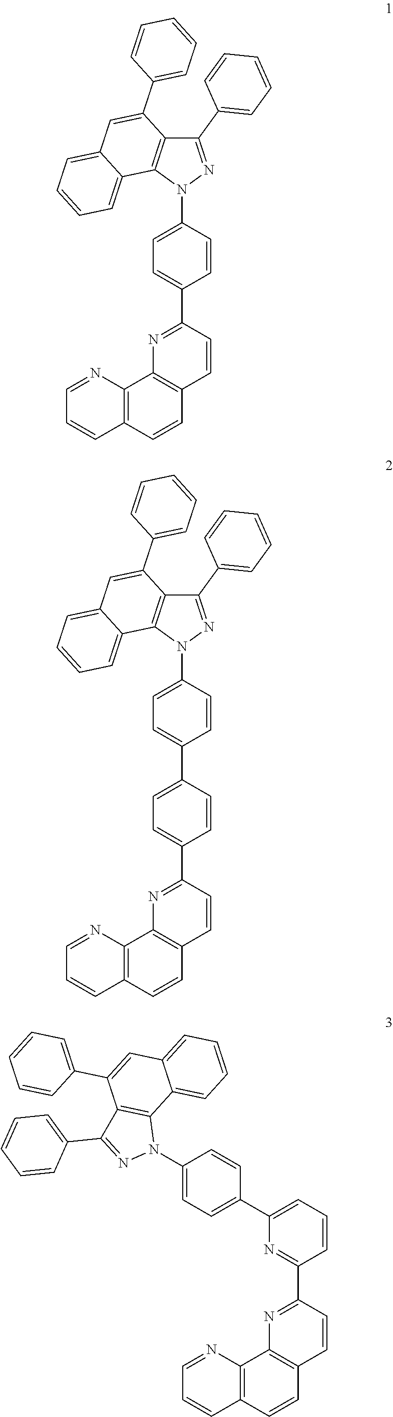

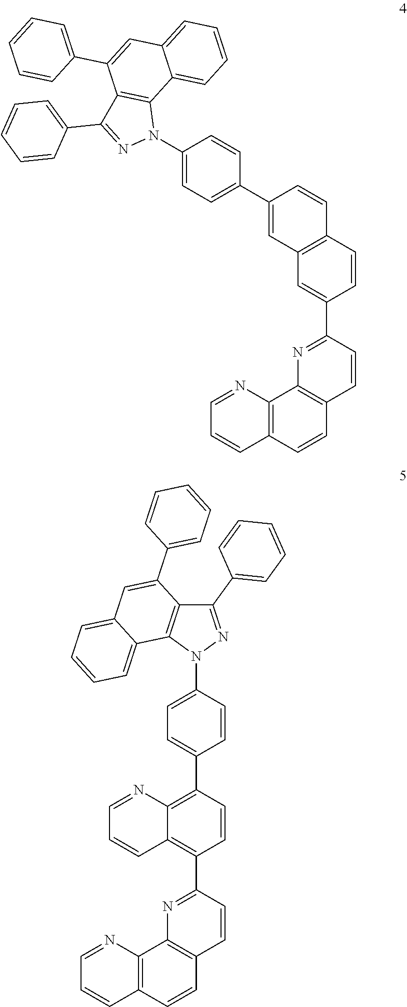 [SMILES: c1ccc(-c2cc3ccccc3c3c2c(-c2ccccc2)nn3-c2ccc(-c3ccc4ccc(-c5ccc6ccc7cccnc7c6n5)cc4c3)cc2)cc1 ; c1ccc(-c2cc3ccccc3c3c2c(-c2ccccc2)nn3-c2ccc(-c3ccc(-c4ccc5ccc6cccnc6c5n4)c4cccnc34)cc2)cc1]

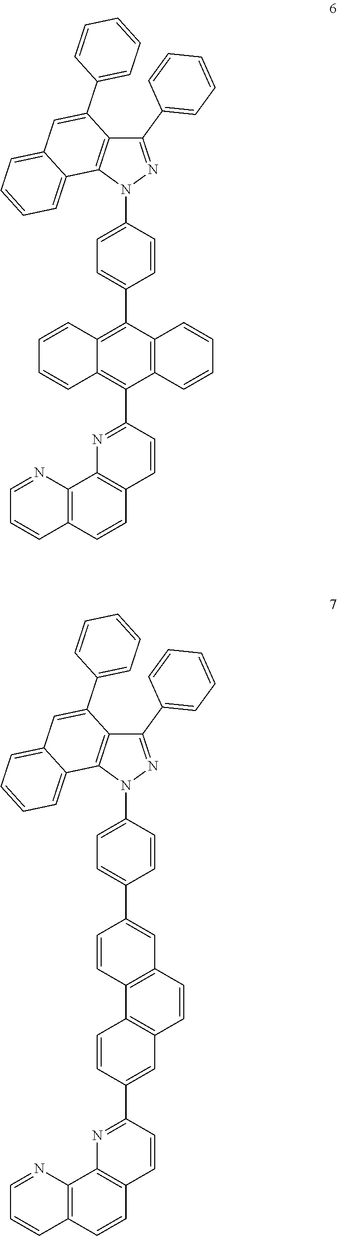 [SMILES: c1ccc(-c2cc3ccccc3c3c2c(-c2ccccc2)nn3-c2ccc(-c3c4ccccc4c(-c4ccc5ccc6cccnc6c5n4)c4ccccc34)cc2)cc1 ; c1ccc(-c2cc3ccccc3c3c2c(-c2ccccc2)nn3-c2ccc(-c3ccc4c(ccc5cc(-c6ccc7ccc8cccnc8c7n6)ccc54)c3)cc2)cc1]

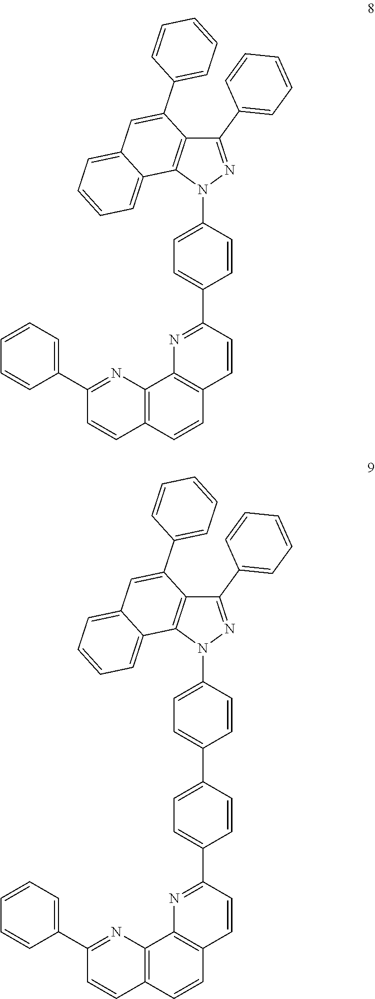

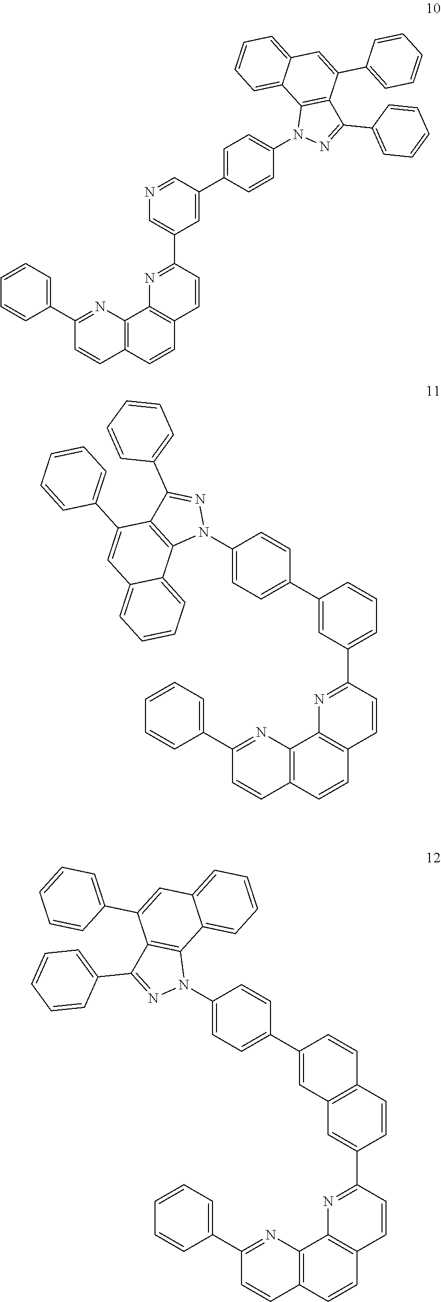

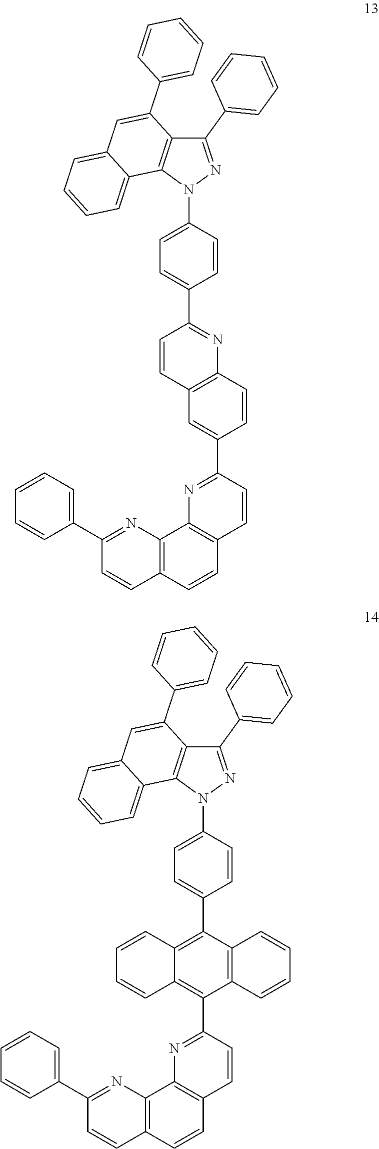

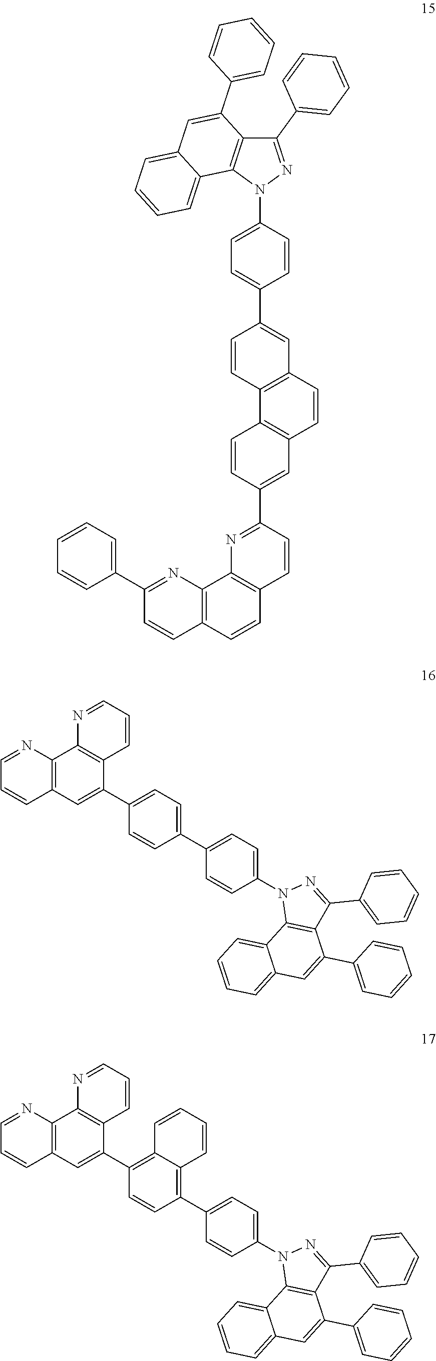 [SMILES: c1ccc(-c2ccc3ccc4ccc(-c5ccc6c(ccc7cc(-c8ccc(-n9nc(-c%10ccccc%10)c%10c(-c%11ccccc%11)cc%11ccccc%11c%109)cc8)ccc76)c5)nc4c3n2)cc1 ; c1ccc(-c2cc3ccccc3c3c2c(-c2ccccc2)nn3-c2ccc(-c3ccc(-c4cc5cccnc5c5ncccc45)cc3)cc2)cc1 ; c1ccc(-c2cc3ccccc3c3c2c(-c2ccccc2)nn3-c2ccc(-c3ccc(-c4cc5cccnc5c5ncccc45)c4ccccc34)cc2)cc1]

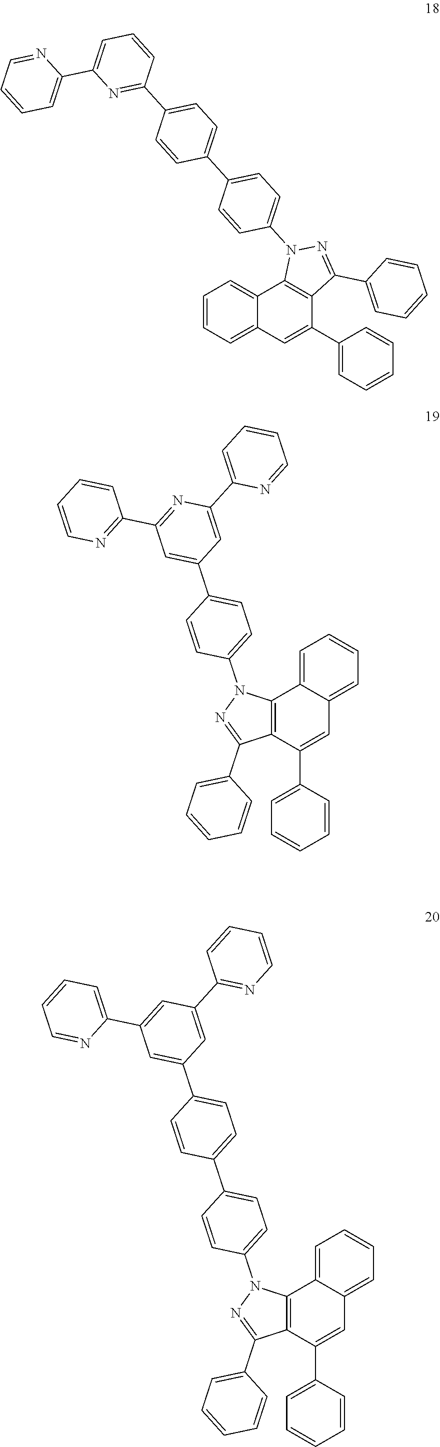 [SMILES: c1ccc(-c2cc3ccccc3c3c2c(-c2ccccc2)nn3-c2ccc(-c3ccc(-c4cccc(-c5ccccn5)n4)cc3)cc2)cc1 ; c1ccc(-c2cc3ccccc3c3c2c(-c2ccccc2)nn3-c2ccc(-c3cc(-c4ccccn4)nc(-c4ccccn4)c3)cc2)cc1 ; c1ccc(-c2cc3ccccc3c3c2c(-c2ccccc2)nn3-c2ccc(-c3ccc(-c4cc(-c5ccccn5)cc(-c5ccccn5)c4)cc3)cc2)cc1]

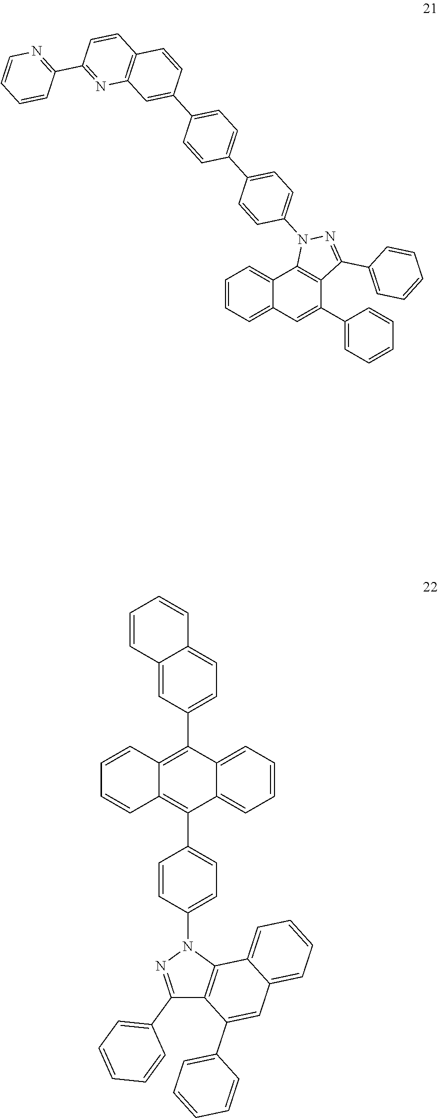 [SMILES: c1ccc(-c2cc3ccccc3c3c2c(-c2ccccc2)nn3-c2ccc(-c3ccc(-c4ccc5ccc(-c6ccccn6)nc5c4)cc3)cc2)cc1 ; c1ccc(-c2cc3ccccc3c3c2c(-c2ccccc2)nn3-c2ccc(-c3c4ccccc4c(-c4ccc5ccccc5c4)c4ccccc34)cc2)cc1]

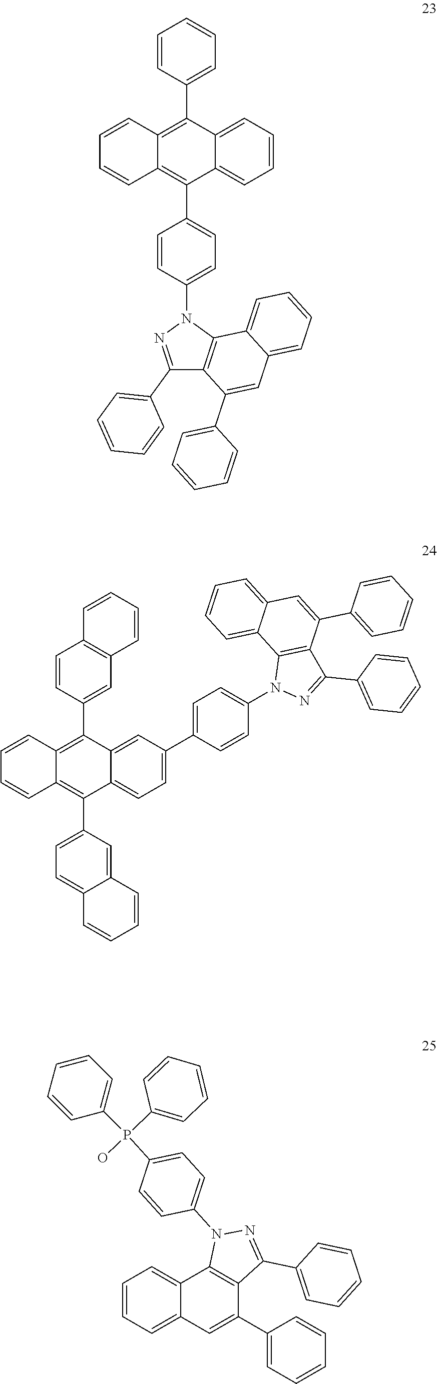

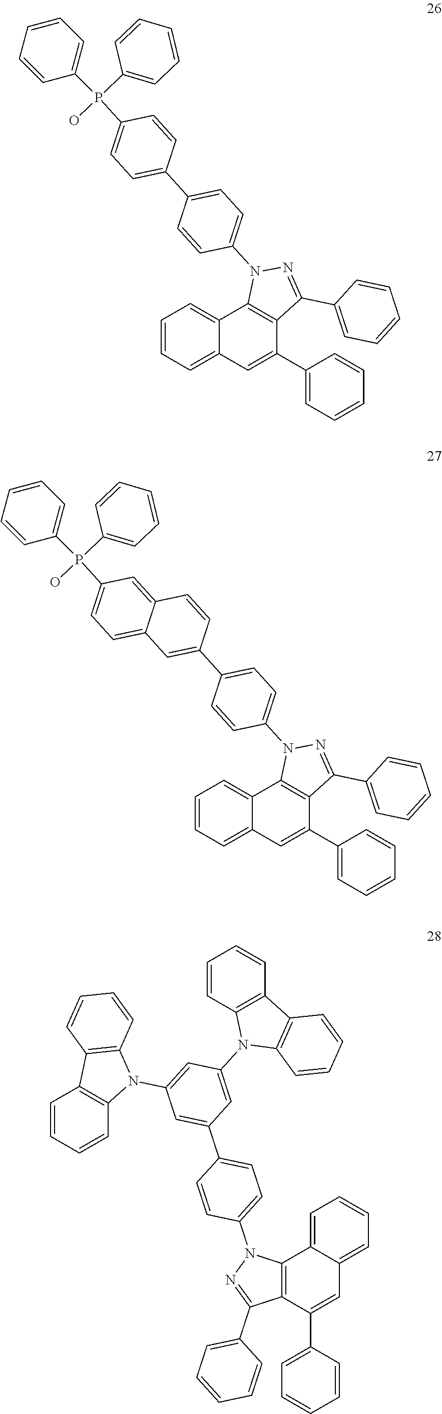

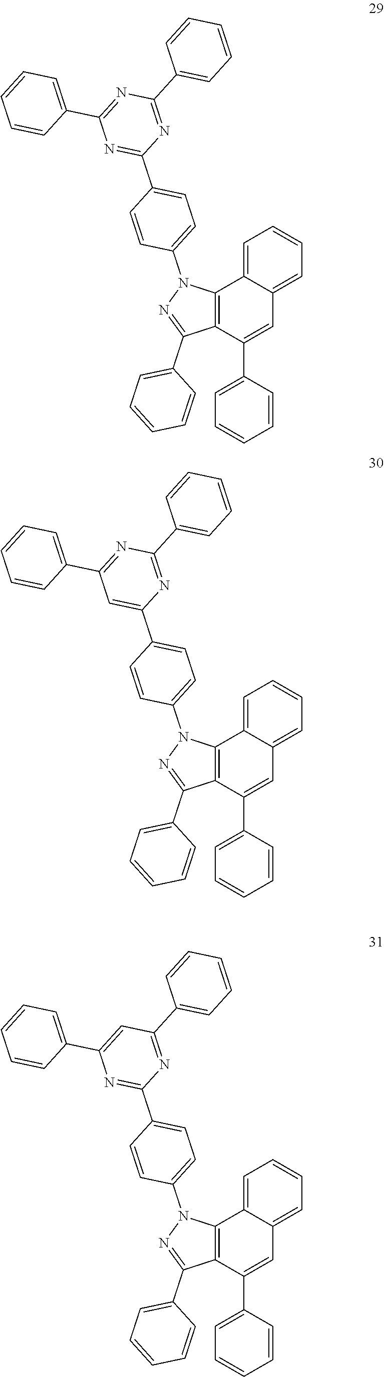

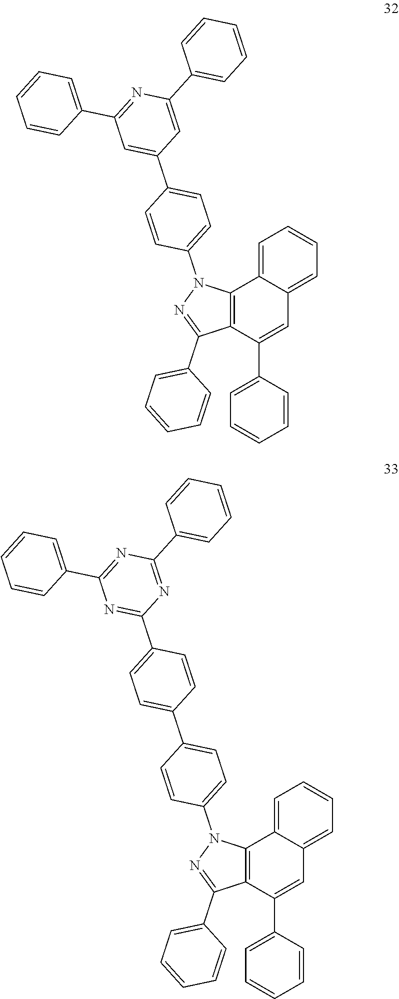 [SMILES: c1ccc(-c2cc(-c3ccc(-n4nc(-c5ccccc5)c5c(-c6ccccc6)cc6ccccc6c54)cc3)cc(-c3ccccc3)n2)cc1 ; c1ccc(-c2nc(-c3ccccc3)nc(-c3ccc(-c4ccc(-n5nc(-c6ccccc6)c6c(-c7ccccc7)cc7ccccc7c65)cc4)cc3)n2)cc1]

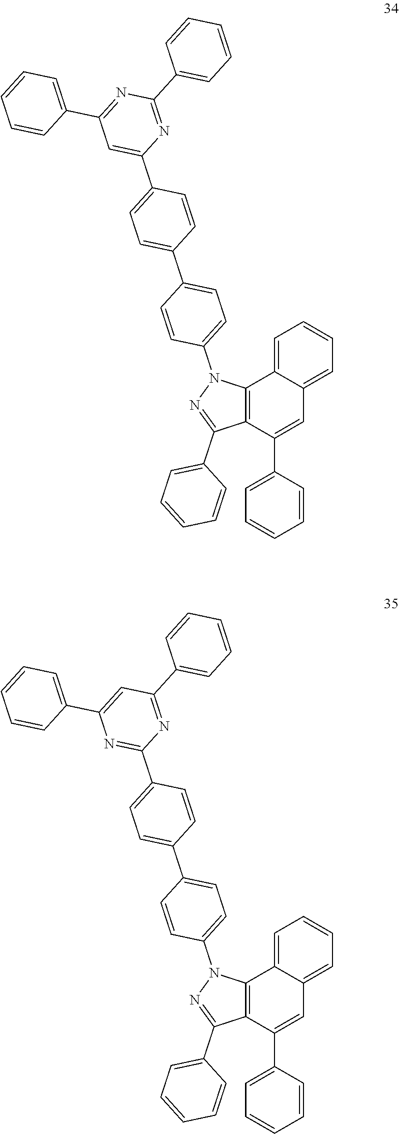 [SMILES: c1ccc(-c2cc(-c3ccc(-c4ccc(-n5nc(-c6ccccc6)c6c(-c7ccccc7)cc7ccccc7c65)cc4)cc3)nc(-c3ccccc3)n2)cc1 ; c1ccc(-c2cc(-c3ccccc3)nc(-c3ccc(-c4ccc(-n5nc(-c6ccccc6)c6c(-c7ccccc7)cc7ccccc7c65)cc4)cc3)n2)cc1]

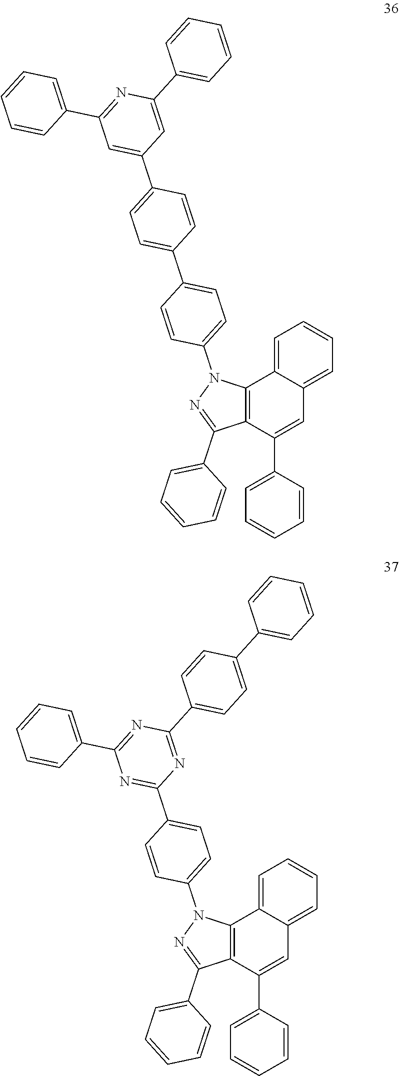

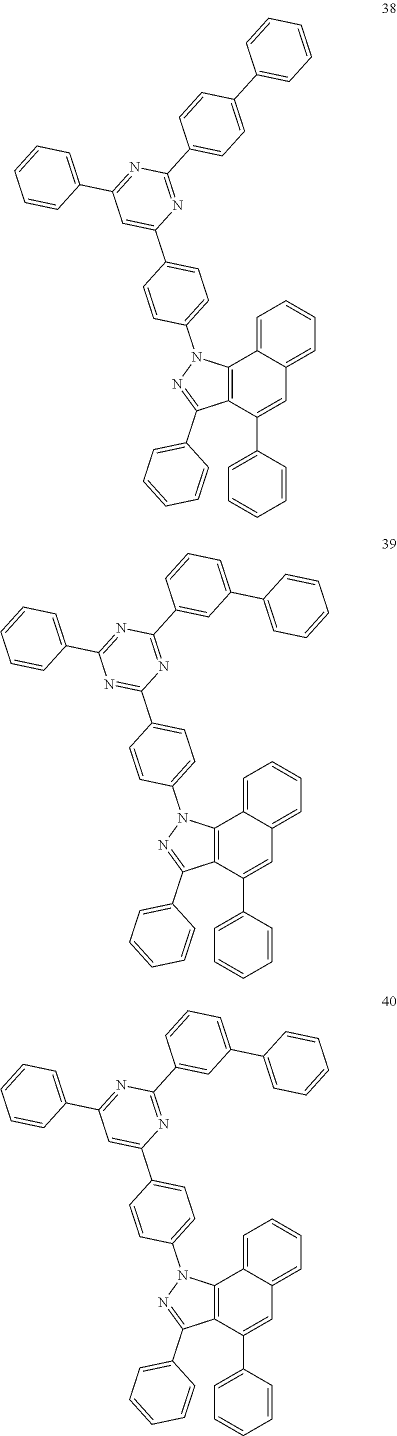

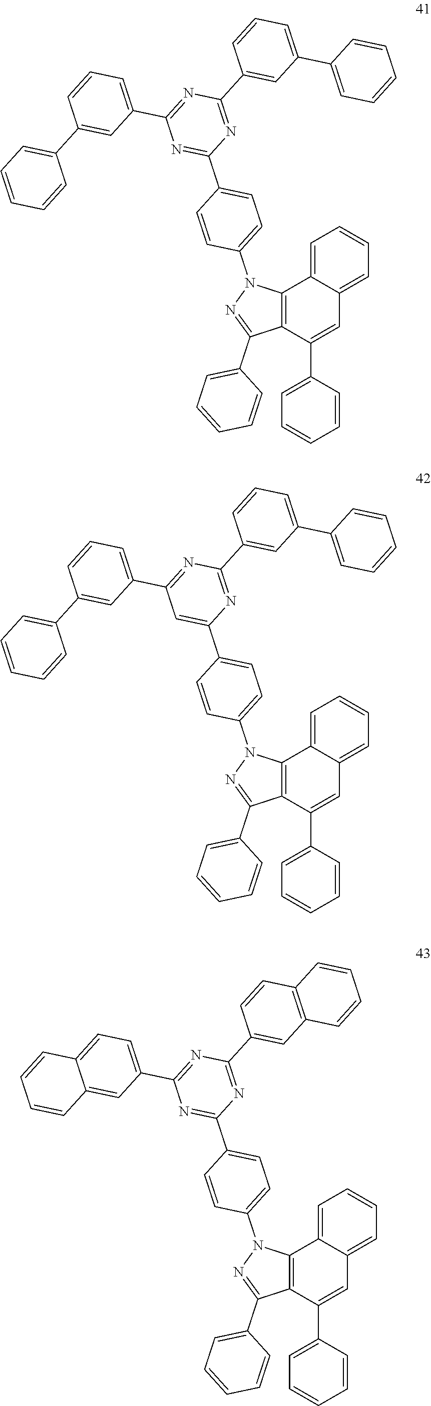 [SMILES: c1ccc(-c2cccc(-c3nc(-c4ccc(-n5nc(-c6ccccc6)c6c(-c7ccccc7)cc7ccccc7c65)cc4)nc(-c4cccc(-c5ccccc5)c4)n3)c2)cc1 ; c1ccc(-c2cccc(-c3cc(-c4ccc(-n5nc(-c6ccccc6)c6c(-c7ccccc7)cc7ccccc7c65)cc4)nc(-c4cccc(-c5ccccc5)c4)n3)c2)cc1 ; c1ccc(-c2cc3ccccc3c3c2c(-c2ccccc2)nn3-c2ccc(-c3nc(-c4ccc5ccccc5c4)nc(-c4ccc5ccccc5c4)n3)cc2)cc1]

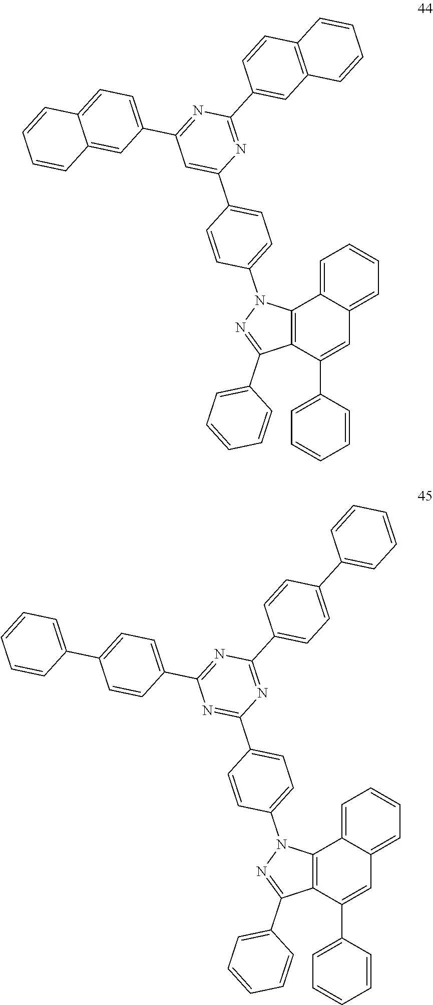 [SMILES: c1ccc(-c2cc3ccccc3c3c2c(-c2ccccc2)nn3-c2ccc(-c3cc(-c4ccc5ccccc5c4)nc(-c4ccc5ccccc5c4)n3)cc2)cc1 ; c1ccc(-c2ccc(-c3nc(-c4ccc(-c5ccccc5)cc4)nc(-c4ccc(-n5nc(-c6ccccc6)c6c(-c7ccccc7)cc7ccccc7c65)cc4)n3)cc2)cc1]

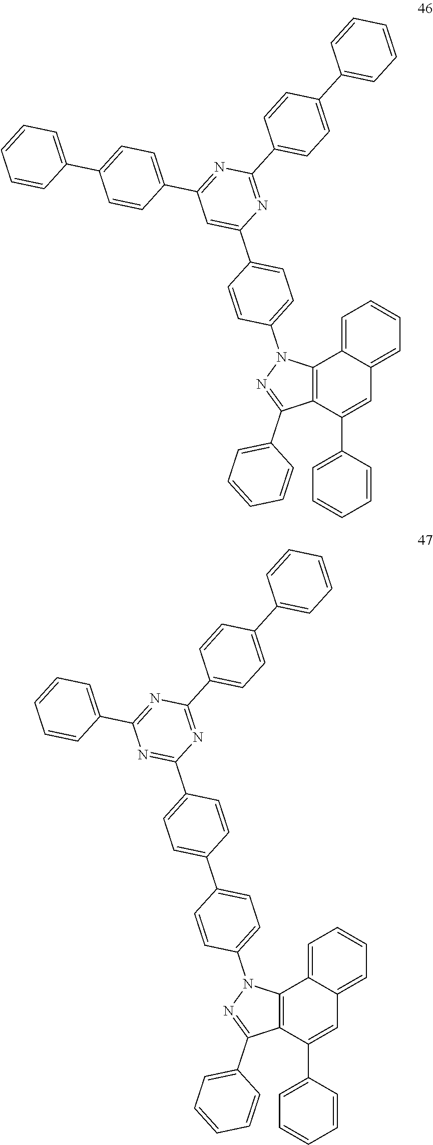

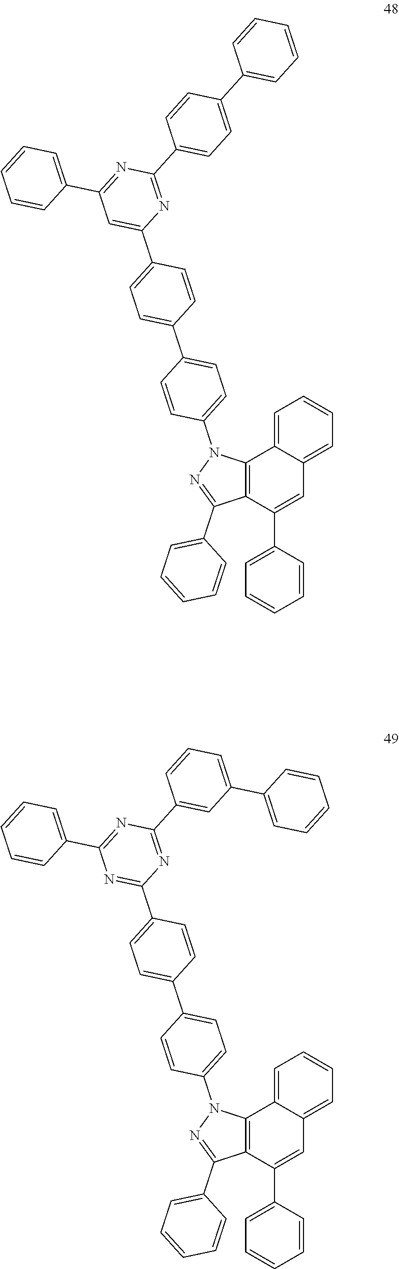 [SMILES: c1ccc(-c2ccc(-c3nc(-c4ccccc4)cc(-c4ccc(-c5ccc(-n6nc(-c7ccccc7)c7c(-c8ccccc8)cc8ccccc8c76)cc5)cc4)n3)cc2)cc1 ; c1ccc(-c2cccc(-c3nc(-c4ccccc4)nc(-c4ccc(-c5ccc(-n6nc(-c7ccccc7)c7c(-c8ccccc8)cc8ccccc8c76)cc5)cc4)n3)c2)cc1]

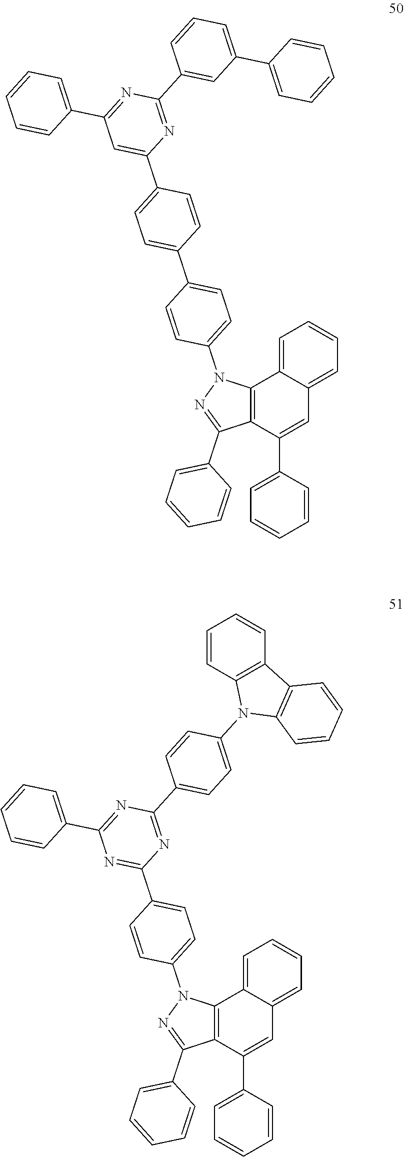 [SMILES: c1ccc(-c2cccc(-c3nc(-c4ccccc4)cc(-c4ccc(-c5ccc(-n6nc(-c7ccccc7)c7c(-c8ccccc8)cc8ccccc8c76)cc5)cc4)n3)c2)cc1 ; c1ccc(-c2nc(-c3ccc(-n4nc(-c5ccccc5)c5c(-c6ccccc6)cc6ccccc6c54)cc3)nc(-c3ccc(-n4c5ccccc5c5ccccc54)cc3)n2)cc1]

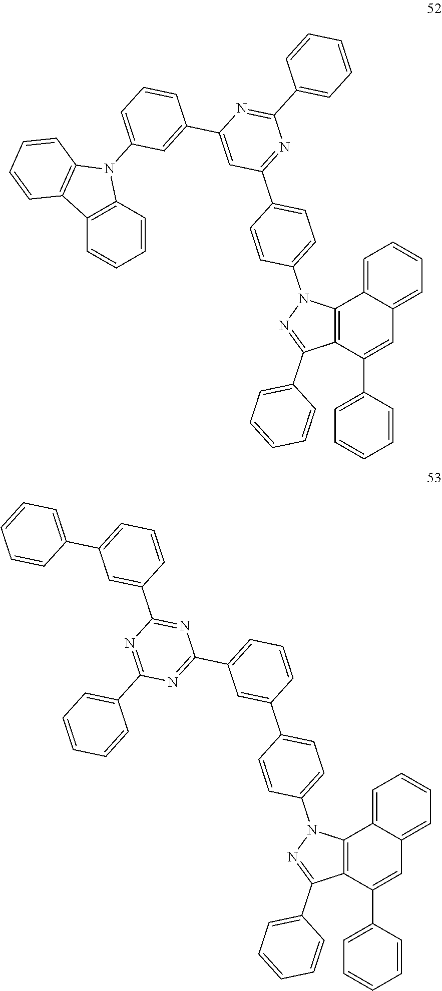 [SMILES: c1ccc(-c2nc(-c3ccc(-n4nc(-c5ccccc5)c5c(-c6ccccc6)cc6ccccc6c54)cc3)cc(-c3cccc(-n4c5ccccc5c5ccccc54)c3)n2)cc1 ; c1ccc(-c2cccc(-c3nc(-c4ccccc4)nc(-c4cccc(-c5ccc(-n6nc(-c7ccccc7)c7c(-c8ccccc8)cc8ccccc8c76)cc5)c4)n3)c2)cc1]

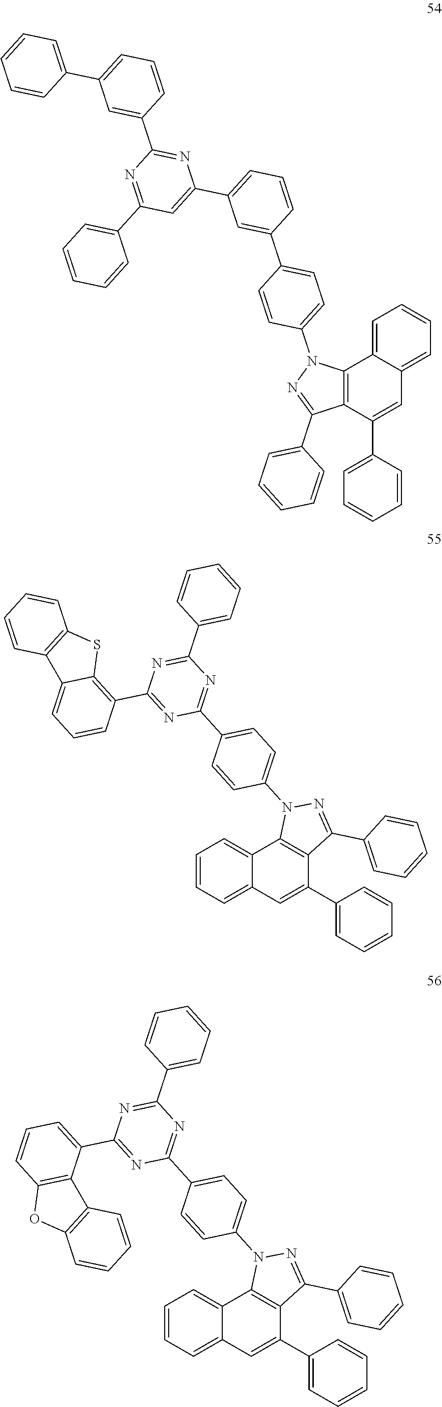 [SMILES: c1ccc(-c2cccc(-c3nc(-c4ccccc4)cc(-c4cccc(-c5ccc(-n6nc(-c7ccccc7)c7c(-c8ccccc8)cc8ccccc8c76)cc5)c4)n3)c2)cc1 ; c1ccc(-c2nc(-c3ccc(-n4nc(-c5ccccc5)c5c(-c6ccccc6)cc6ccccc6c54)cc3)nc(-c3cccc4c3sc3ccccc34)n2)cc1 ; c1ccc(-c2nc(-c3ccc(-n4nc(-c5ccccc5)c5c(-c6ccccc6)cc6ccccc6c54)cc3)nc(-c3cccc4oc5ccccc5c34)n2)cc1]

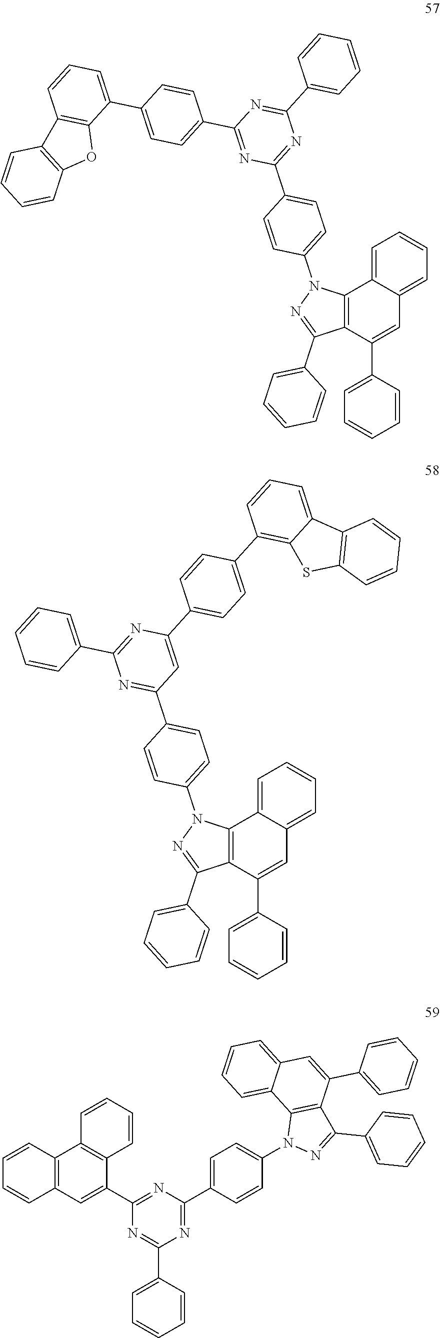 [SMILES: c1ccc(-c2nc(-c3ccc(-c4cccc5c4oc4ccccc45)cc3)nc(-c3ccc(-n4nc(-c5ccccc5)c5c(-c6ccccc6)cc6ccccc6c54)cc3)n2)cc1 ; c1ccc(-c2nc(-c3ccc(-c4cccc5c4sc4ccccc45)cc3)cc(-c3ccc(-n4nc(-c5ccccc5)c5c(-c6ccccc6)cc6ccccc6c54)cc3)n2)cc1 ; c1ccc(-c2nc(-c3ccc(-n4nc(-c5ccccc5)c5c(-c6ccccc6)cc6ccccc6c54)cc3)nc(-c3cc4ccccc4c4ccccc34)n2)cc1]

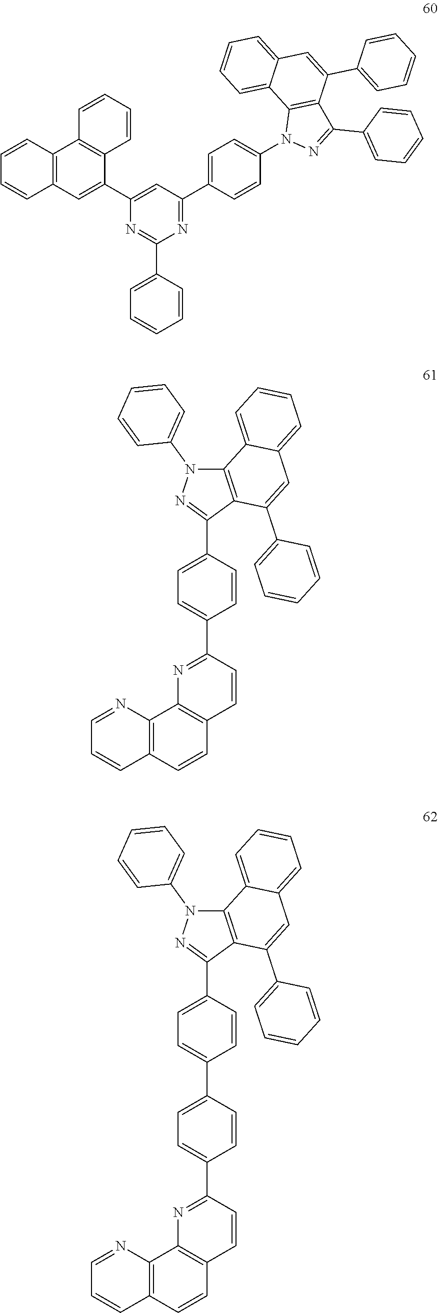 [SMILES: c1ccc(-c2nc(-c3ccc(-n4nc(-c5ccccc5)c5c(-c6ccccc6)cc6ccccc6c54)cc3)cc(-c3cc4ccccc4c4ccccc34)n2)cc1 ; c1ccc(-c2cc3ccccc3c3c2c(-c2ccc(-c4ccc5ccc6cccnc6c5n4)cc2)nn3-c2ccccc2)cc1 ; c1ccc(-c2cc3ccccc3c3c2c(-c2ccc(-c4ccc(-c5ccc6ccc7cccnc7c6n5)cc4)cc2)nn3-c2ccccc2)cc1]

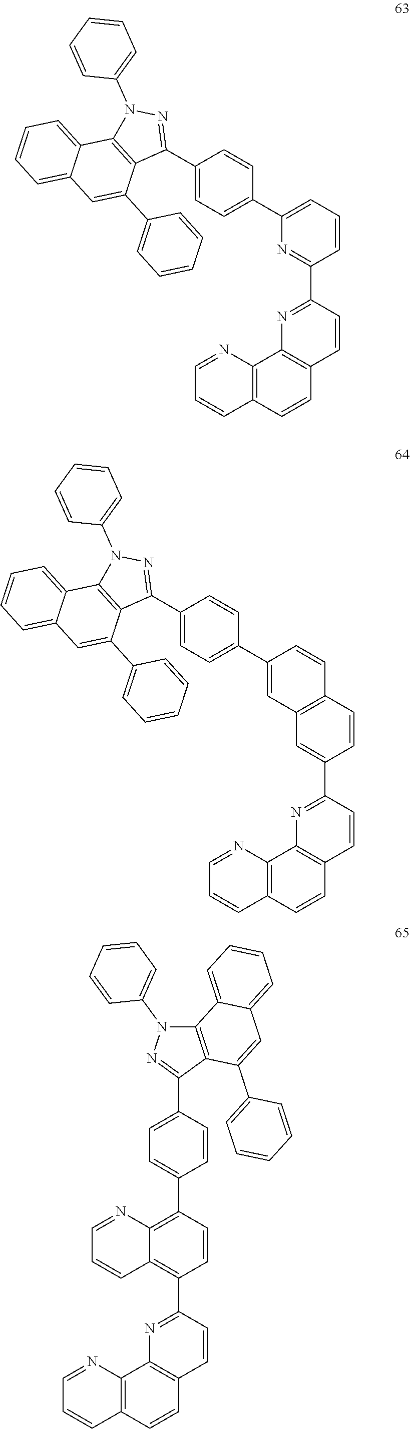

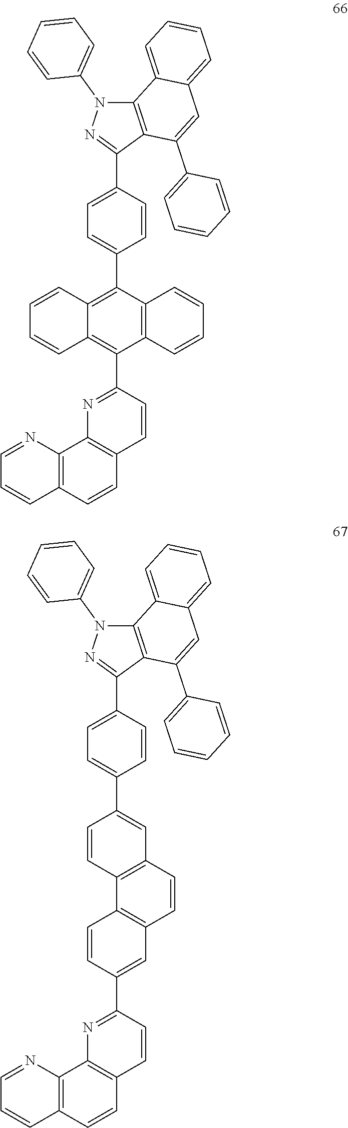

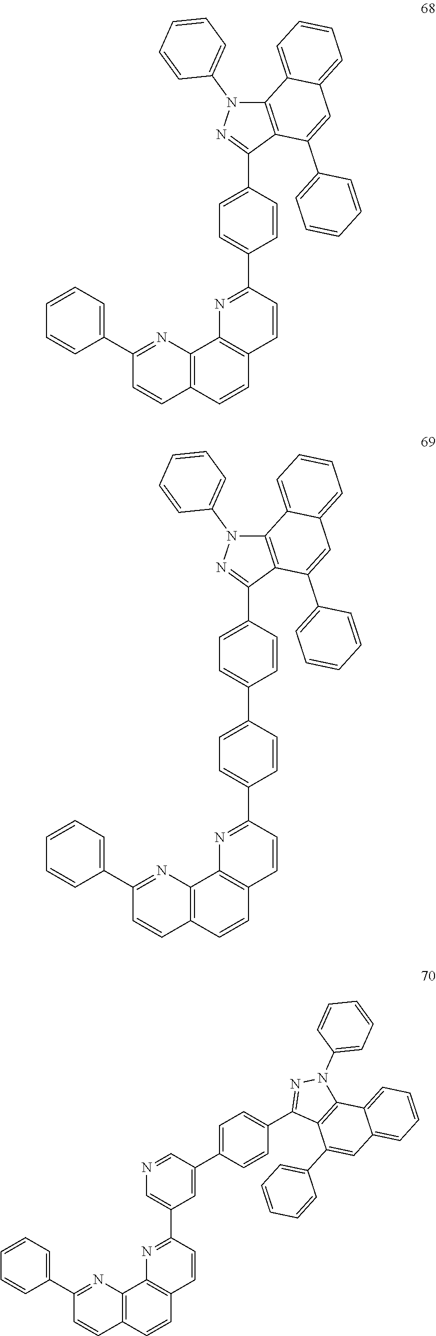 [SMILES: c1ccc(-c2ccc3ccc4ccc(-c5ccc(-c6nn(-c7ccccc7)c7c6c(-c6ccccc6)cc6ccccc67)cc5)nc4c3n2)cc1 ; c1ccc(-c2ccc3ccc4ccc(-c5ccc(-c6ccc(-c7nn(-c8ccccc8)c8c7c(-c7ccccc7)cc7ccccc78)cc6)cc5)nc4c3n2)cc1 ; c1ccc(-c2ccc3ccc4ccc(-c5cncc(-c6ccc(-c7nn(-c8ccccc8)c8c7c(-c7ccccc7)cc7ccccc78)cc6)c5)nc4c3n2)cc1]

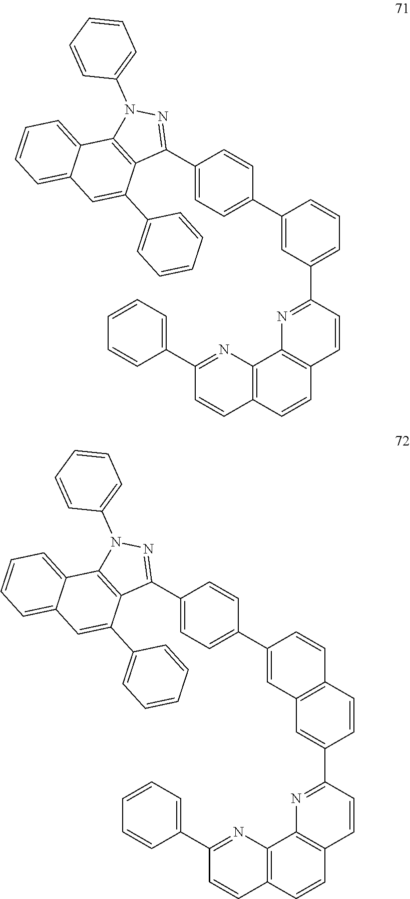 [SMILES: c1ccc(-c2ccc3ccc4ccc(-c5cccc(-c6ccc(-c7nn(-c8ccccc8)c8c7c(-c7ccccc7)cc7ccccc78)cc6)c5)nc4c3n2)cc1 ; c1ccc(-c2ccc3ccc4ccc(-c5ccc6ccc(-c7ccc(-c8nn(-c9ccccc9)c9c8c(-c8ccccc8)cc8ccccc89)cc7)cc6c5)nc4c3n2)cc1]

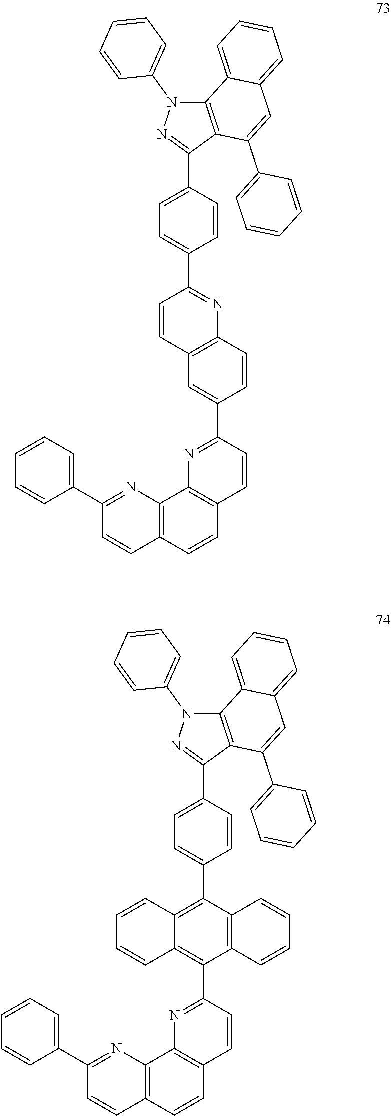 [SMILES: c1ccc(-c2ccc3ccc4ccc(-c5ccc6nc(-c7ccc(-c8nn(-c9ccccc9)c9c8c(-c8ccccc8)cc8ccccc89)cc7)ccc6c5)nc4c3n2)cc1 ; c1ccc(-c2ccc3ccc4ccc(-c5c6ccccc6c(-c6ccc(-c7nn(-c8ccccc8)c8c7c(-c7ccccc7)cc7ccccc78)cc6)c6ccccc56)nc4c3n2)cc1]

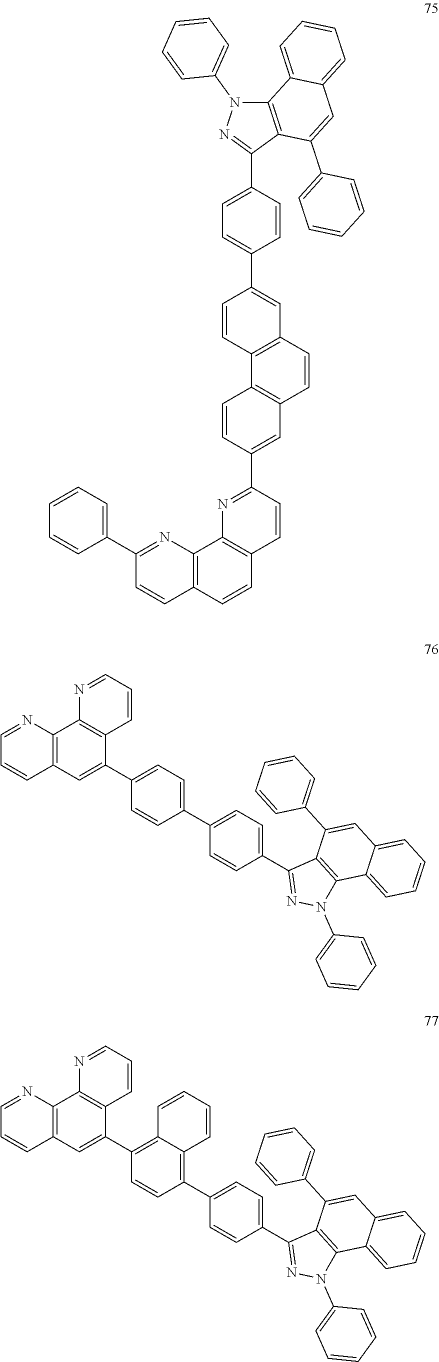 [SMILES: c1ccc(-c2ccc3ccc4ccc(-c5ccc6c(ccc7cc(-c8ccc(-c9nn(-c%10ccccc%10)c%10c9c(-c9ccccc9)cc9ccccc9%10)cc8)ccc76)c5)nc4c3n2)cc1 ; c1ccc(-c2cc3ccccc3c3c2c(-c2ccc(-c4ccc(-c5cc6cccnc6c6ncccc56)cc4)cc2)nn3-c2ccccc2)cc1 ; c1ccc(-c2cc3ccccc3c3c2c(-c2ccc(-c4ccc(-c5cc6cccnc6c6ncccc56)c5ccccc45)cc2)nn3-c2ccccc2)cc1]

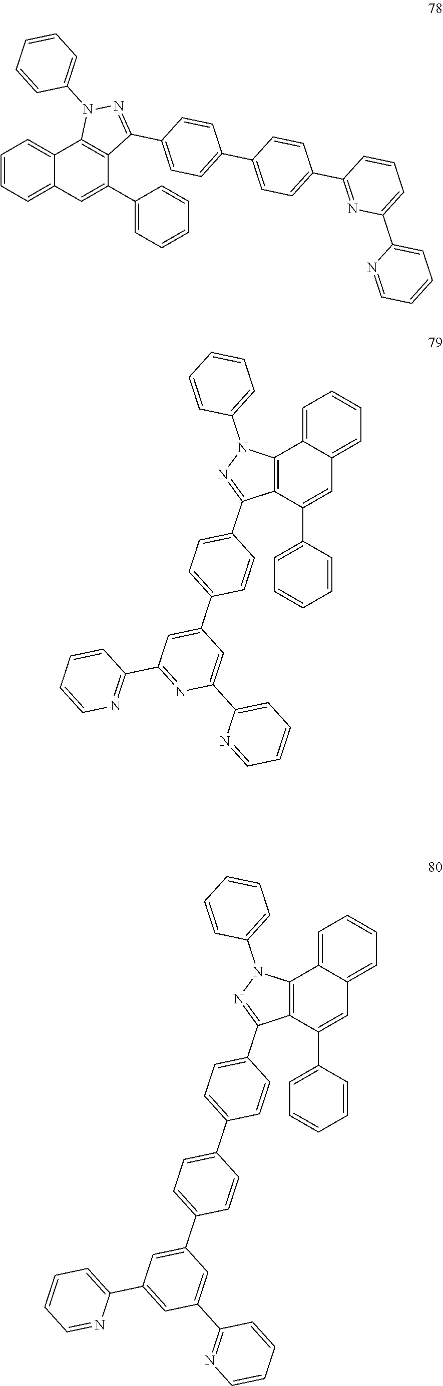 [SMILES: c1ccc(-c2cc3ccccc3c3c2c(-c2ccc(-c4ccc(-c5cccc(-c6ccccn6)n5)cc4)cc2)nn3-c2ccccc2)cc1 ; c1ccc(-c2cc3ccccc3c3c2c(-c2ccc(-c4cc(-c5ccccn5)nc(-c5ccccn5)c4)cc2)nn3-c2ccccc2)cc1 ; c1ccc(-c2cc3ccccc3c3c2c(-c2ccc(-c4ccc(-c5cc(-c6ccccn6)cc(-c6ccccn6)c5)cc4)cc2)nn3-c2ccccc2)cc1]

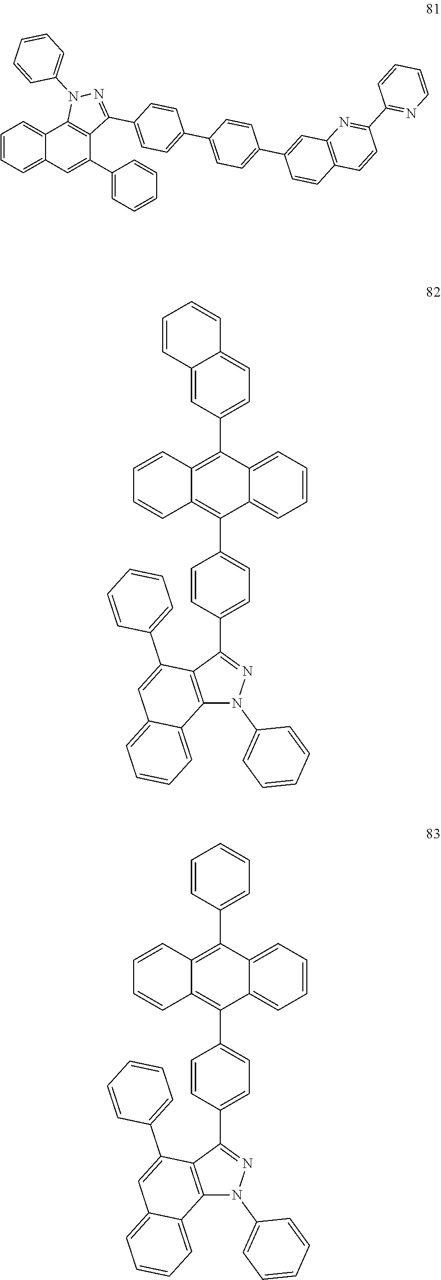 [SMILES: c1ccc(-c2cc3ccccc3c3c2c(-c2ccc(-c4ccc(-c5ccc6ccc(-c7ccccn7)nc6c5)cc4)cc2)nn3-c2ccccc2)cc1 ; c1ccc(-c2cc3ccccc3c3c2c(-c2ccc(-c4c5ccccc5c(-c5ccc6ccccc6c5)c5ccccc45)cc2)nn3-c2ccccc2)cc1 ; c1ccc(-c2c3ccccc3c(-c3ccc(-c4nn(-c5ccccc5)c5c4c(-c4ccccc4)cc4ccccc45)cc3)c3ccccc23)cc1]

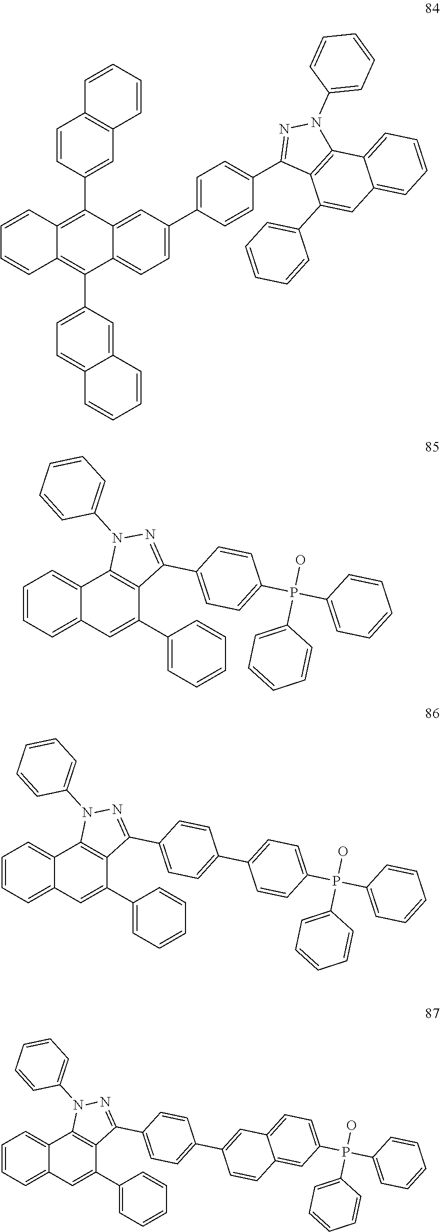

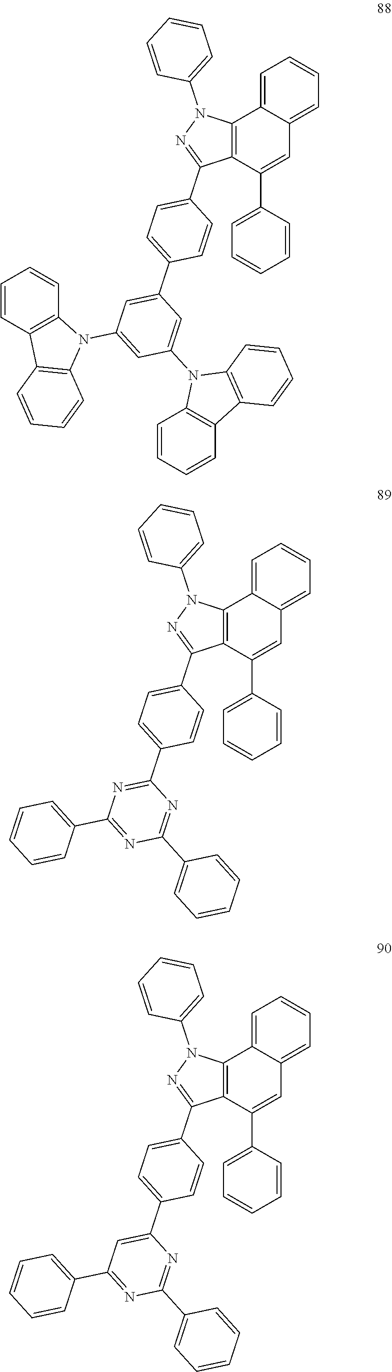

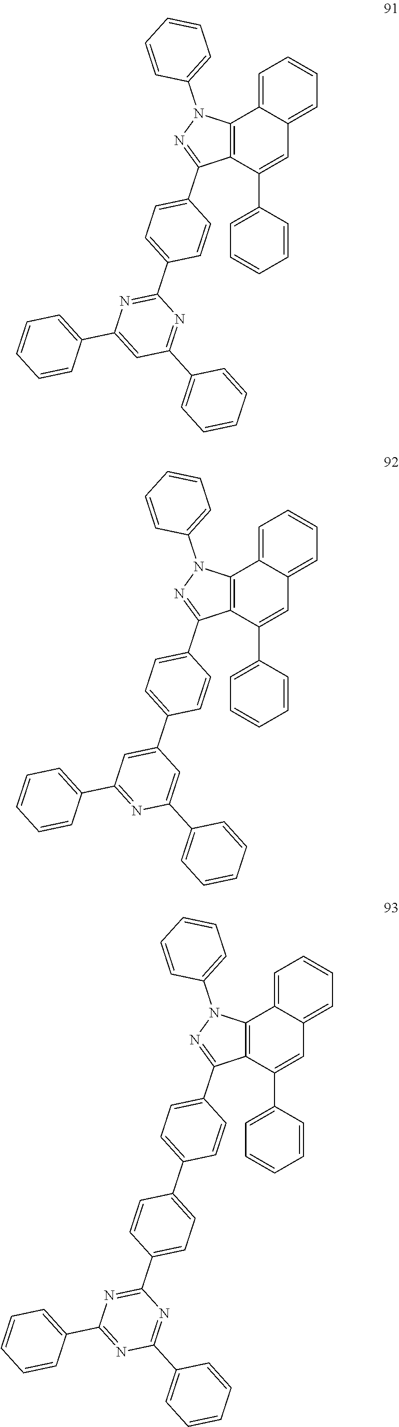

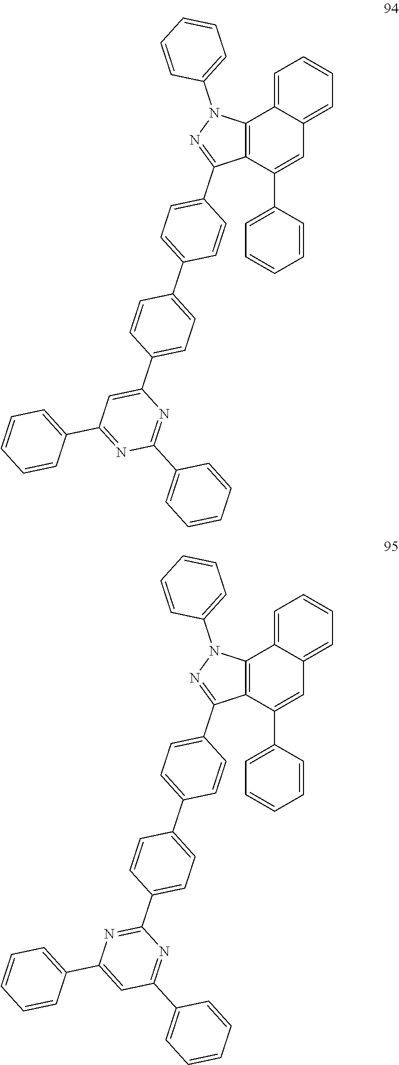

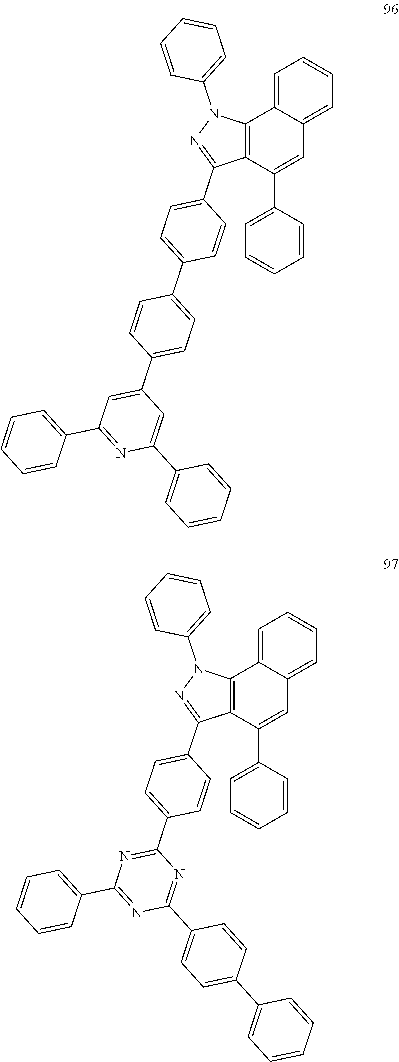 [SMILES: c1ccc(-c2cc(-c3ccc(-c4ccc(-c5nn(-c6ccccc6)c6c5c(-c5ccccc5)cc5ccccc56)cc4)cc3)cc(-c3ccccc3)n2)cc1 ; c1ccc(-c2ccc(-c3nc(-c4ccccc4)nc(-c4ccc(-c5nn(-c6ccccc6)c6c5c(-c5ccccc5)cc5ccccc56)cc4)n3)cc2)cc1]

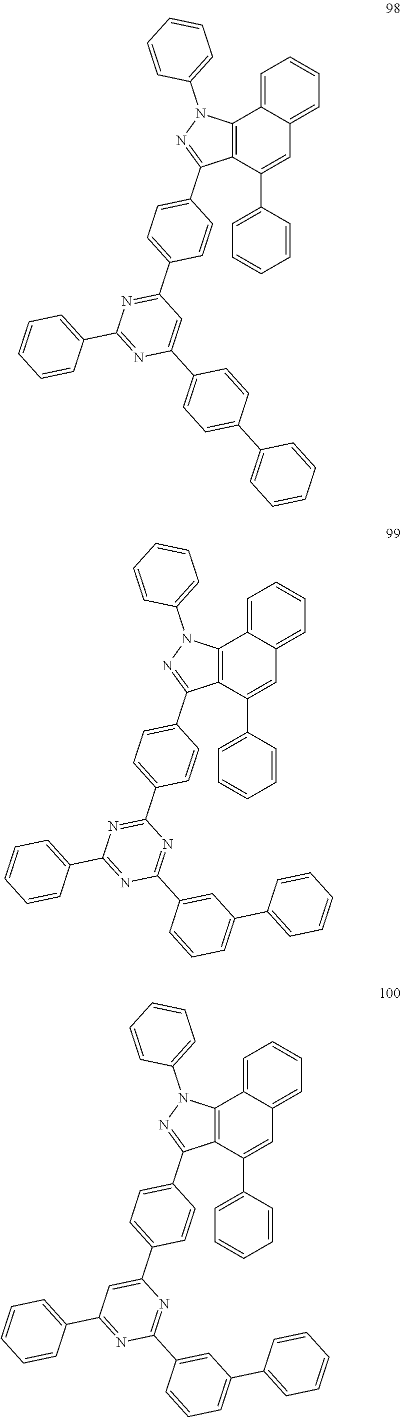 [SMILES: c1ccc(-c2ccc(-c3cc(-c4ccc(-c5nn(-c6ccccc6)c6c5c(-c5ccccc5)cc5ccccc56)cc4)nc(-c4ccccc4)n3)cc2)cc1 ; c1ccc(-c2cccc(-c3nc(-c4ccccc4)nc(-c4ccc(-c5nn(-c6ccccc6)c6c5c(-c5ccccc5)cc5ccccc56)cc4)n3)c2)cc1 ; c1ccc(-c2cccc(-c3nc(-c4ccccc4)cc(-c4ccc(-c5nn(-c6ccccc6)c6c5c(-c5ccccc5)cc5ccccc56)cc4)n3)c2)cc1]

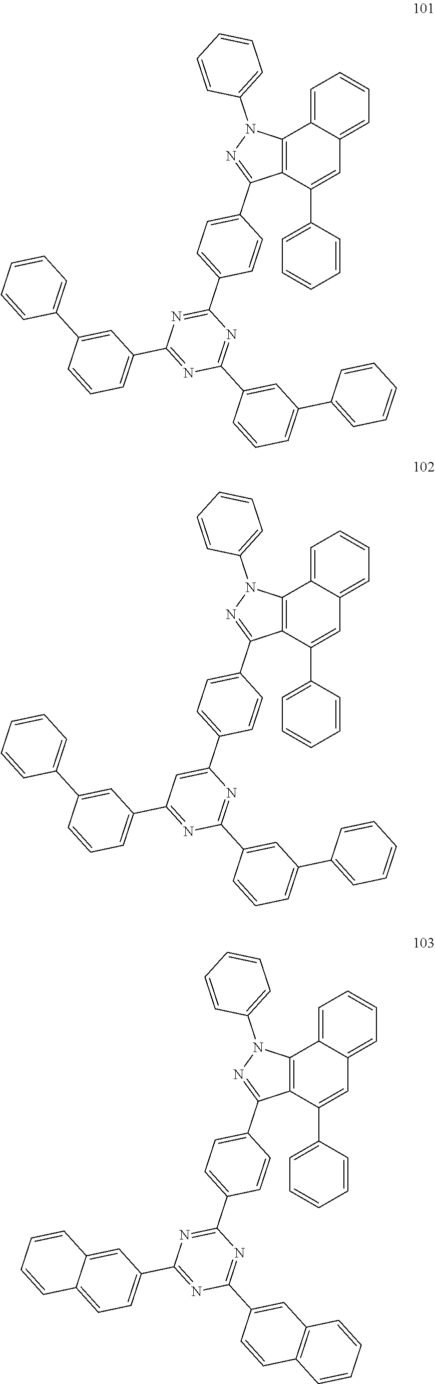 [SMILES: c1ccc(-c2cccc(-c3nc(-c4ccc(-c5nn(-c6ccccc6)c6c5c(-c5ccccc5)cc5ccccc56)cc4)nc(-c4cccc(-c5ccccc5)c4)n3)c2)cc1 ; c1ccc(-c2cccc(-c3cc(-c4ccc(-c5nn(-c6ccccc6)c6c5c(-c5ccccc5)cc5ccccc56)cc4)nc(-c4cccc(-c5ccccc5)c4)n3)c2)cc1 ; c1ccc(-c2cc3ccccc3c3c2c(-c2ccc(-c4nc(-c5ccc6ccccc6c5)nc(-c5ccc6ccccc6c5)n4)cc2)nn3-c2ccccc2)cc1]

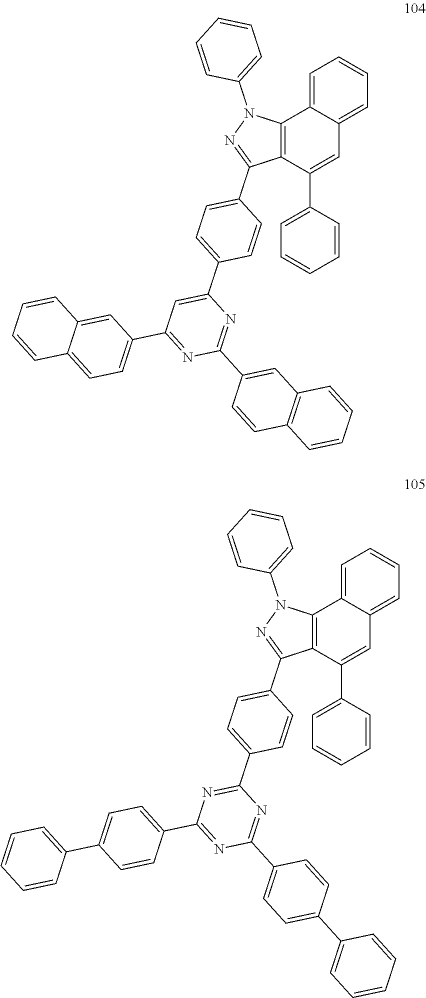 [SMILES: c1ccc(-c2cc3ccccc3c3c2c(-c2ccc(-c4cc(-c5ccc6ccccc6c5)nc(-c5ccc6ccccc6c5)n4)cc2)nn3-c2ccccc2)cc1 ; c1ccc(-c2ccc(-c3nc(-c4ccc(-c5ccccc5)cc4)nc(-c4ccc(-c5nn(-c6ccccc6)c6c5c(-c5ccccc5)cc5ccccc56)cc4)n3)cc2)cc1]

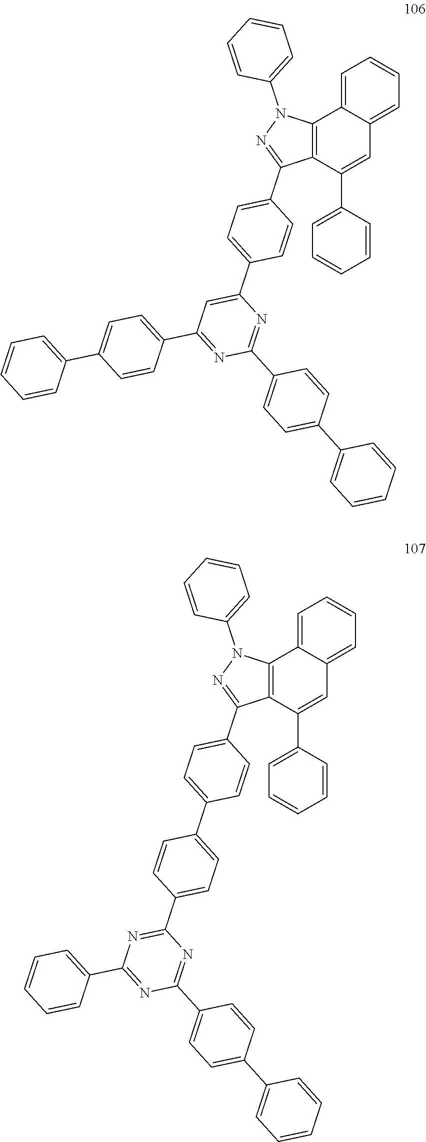 [SMILES: c1ccc(-c2ccc(-c3cc(-c4ccc(-c5nn(-c6ccccc6)c6c5c(-c5ccccc5)cc5ccccc56)cc4)nc(-c4ccc(-c5ccccc5)cc4)n3)cc2)cc1 ; c1ccc(-c2ccc(-c3nc(-c4ccccc4)nc(-c4ccc(-c5ccc(-c6nn(-c7ccccc7)c7c6c(-c6ccccc6)cc6ccccc67)cc5)cc4)n3)cc2)cc1]

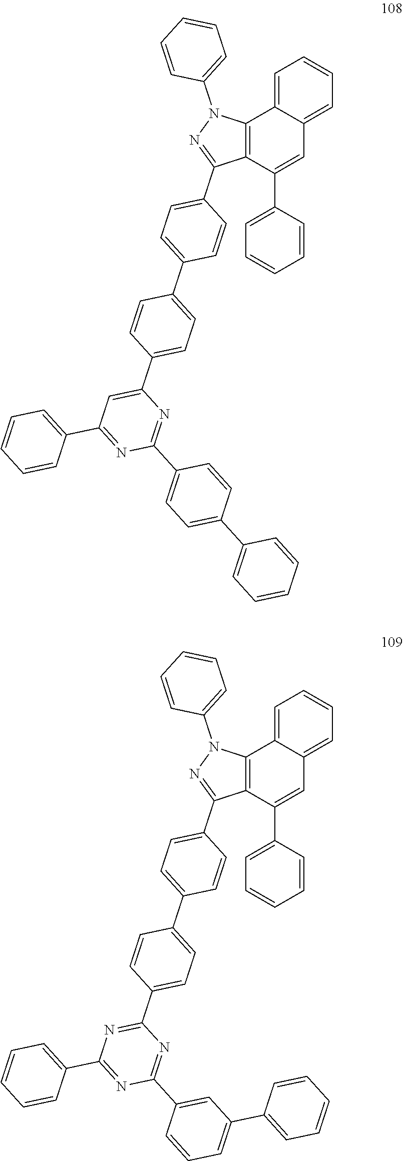 [SMILES: c1ccc(-c2ccc(-c3nc(-c4ccccc4)cc(-c4ccc(-c5ccc(-c6nn(-c7ccccc7)c7c6c(-c6ccccc6)cc6ccccc67)cc5)cc4)n3)cc2)cc1 ; c1ccc(-c2cccc(-c3nc(-c4ccccc4)nc(-c4ccc(-c5ccc(-c6nn(-c7ccccc7)c7c6c(-c6ccccc6)cc6ccccc67)cc5)cc4)n3)c2)cc1]

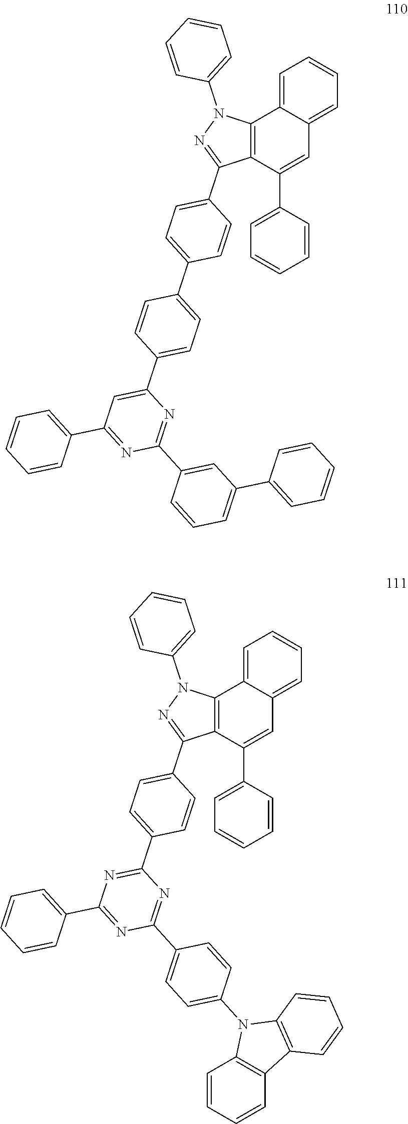 [SMILES: c1ccc(-c2cccc(-c3nc(-c4ccccc4)cc(-c4ccc(-c5ccc(-c6nn(-c7ccccc7)c7c6c(-c6ccccc6)cc6ccccc67)cc5)cc4)n3)c2)cc1 ; c1ccc(-c2nc(-c3ccc(-c4nn(-c5ccccc5)c5c4c(-c4ccccc4)cc4ccccc45)cc3)nc(-c3ccc(-n4c5ccccc5c5ccccc54)cc3)n2)cc1]

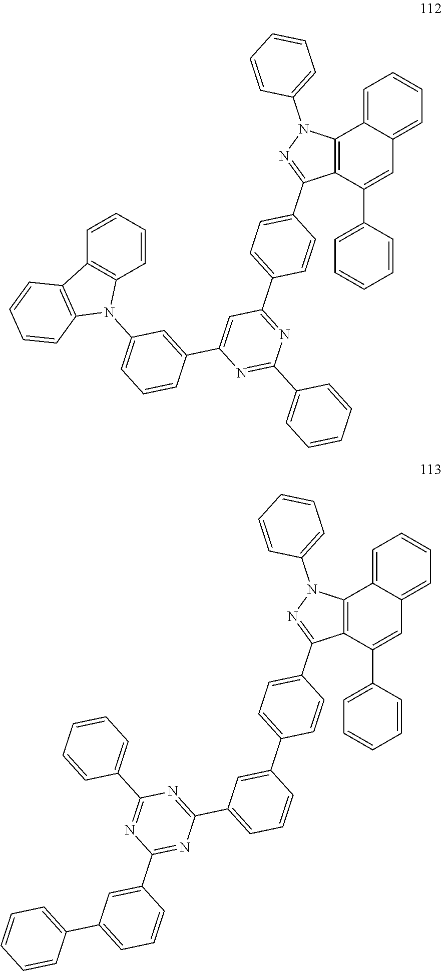 [SMILES: c1ccc(-c2nc(-c3ccc(-c4nn(-c5ccccc5)c5c4c(-c4ccccc4)cc4ccccc45)cc3)cc(-c3cccc(-n4c5ccccc5c5ccccc54)c3)n2)cc1 ; c1ccc(-c2cccc(-c3nc(-c4ccccc4)nc(-c4cccc(-c5ccc(-c6nn(-c7ccccc7)c7c6c(-c6ccccc6)cc6ccccc67)cc5)c4)n3)c2)cc1]

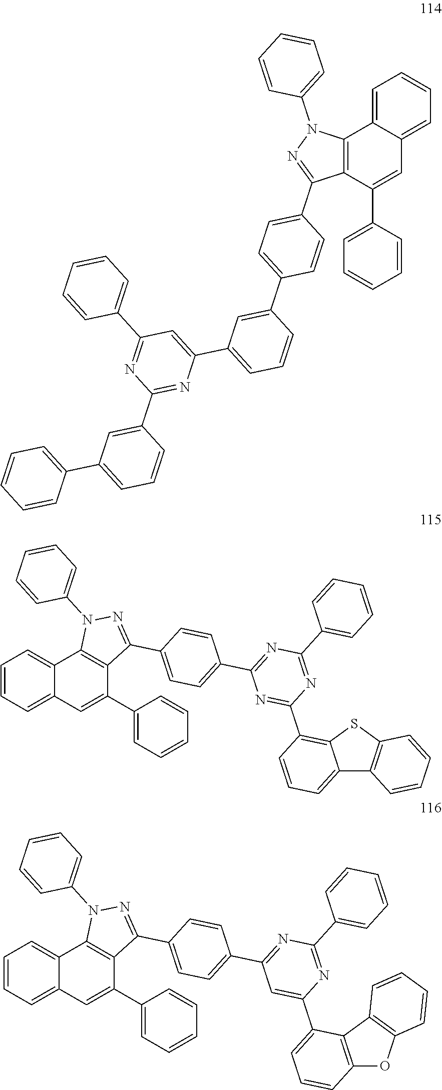

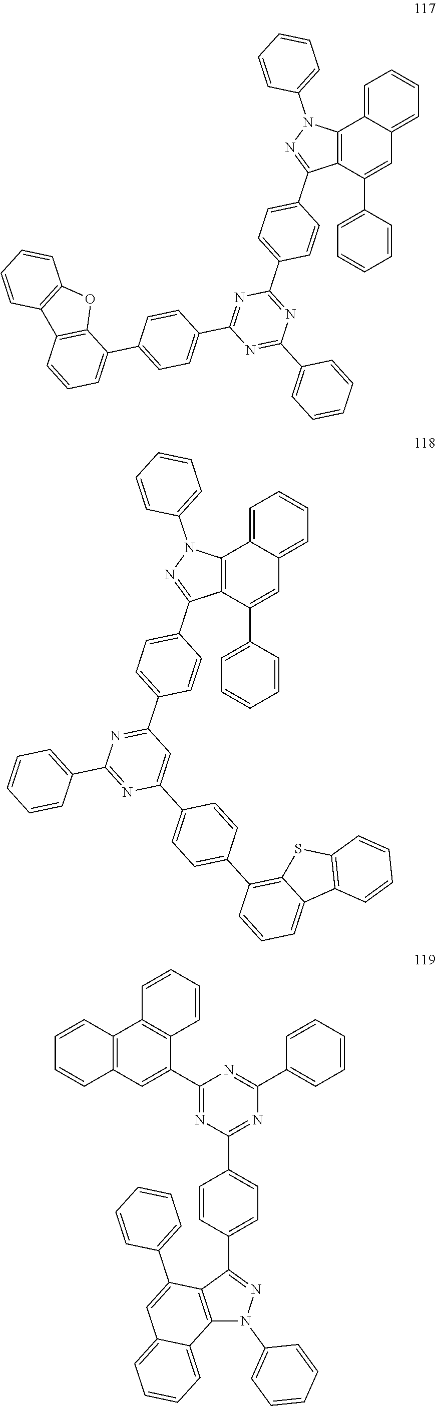 [SMILES: c1ccc(-c2nc(-c3ccc(-c4cccc5c4oc4ccccc45)cc3)nc(-c3ccc(-c4nn(-c5ccccc5)c5c4c(-c4ccccc4)cc4ccccc45)cc3)n2)cc1 ; c1ccc(-c2nc(-c3ccc(-c4cccc5c4sc4ccccc45)cc3)cc(-c3ccc(-c4nn(-c5ccccc5)c5c4c(-c4ccccc4)cc4ccccc45)cc3)n2)cc1 ; c1ccc(-c2nc(-c3ccc(-c4nn(-c5ccccc5)c5c4c(-c4ccccc4)cc4ccccc45)cc3)nc(-c3cc4ccccc4c4ccccc34)n2)cc1]

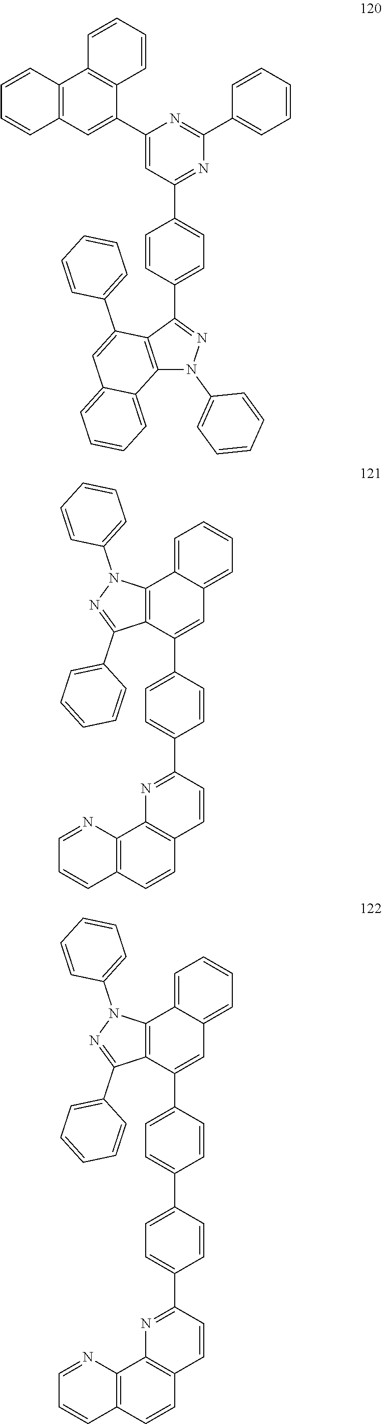

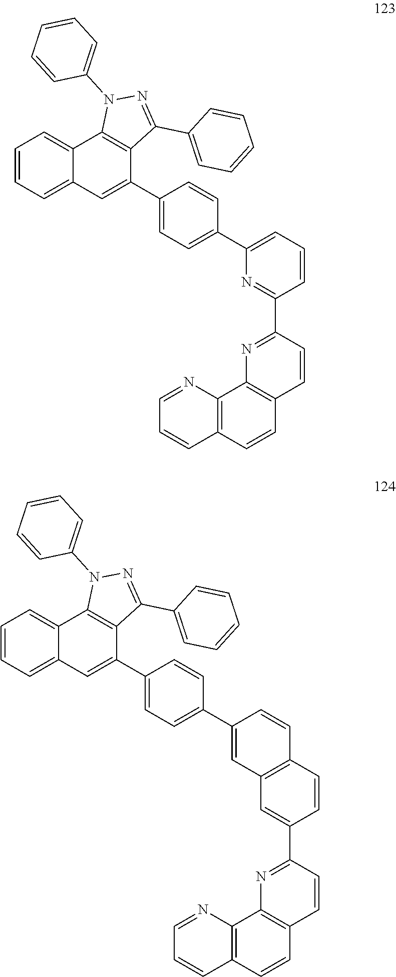 [SMILES: c1ccc(-c2nn(-c3ccccc3)c3c2c(-c2ccc(-c4cccc(-c5ccc6ccc7cccnc7c6n5)n4)cc2)cc2ccccc23)cc1 ; c1ccc(-c2nn(-c3ccccc3)c3c2c(-c2ccc(-c4ccc5ccc(-c6ccc7ccc8cccnc8c7n6)cc5c4)cc2)cc2ccccc23)cc1]

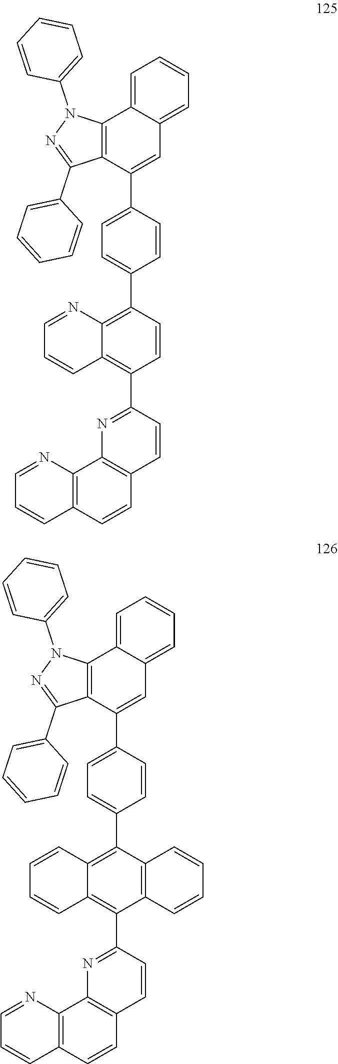

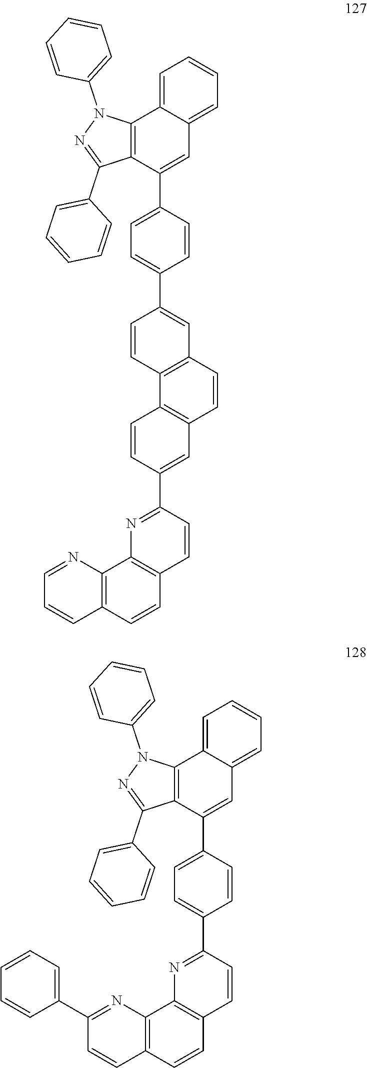

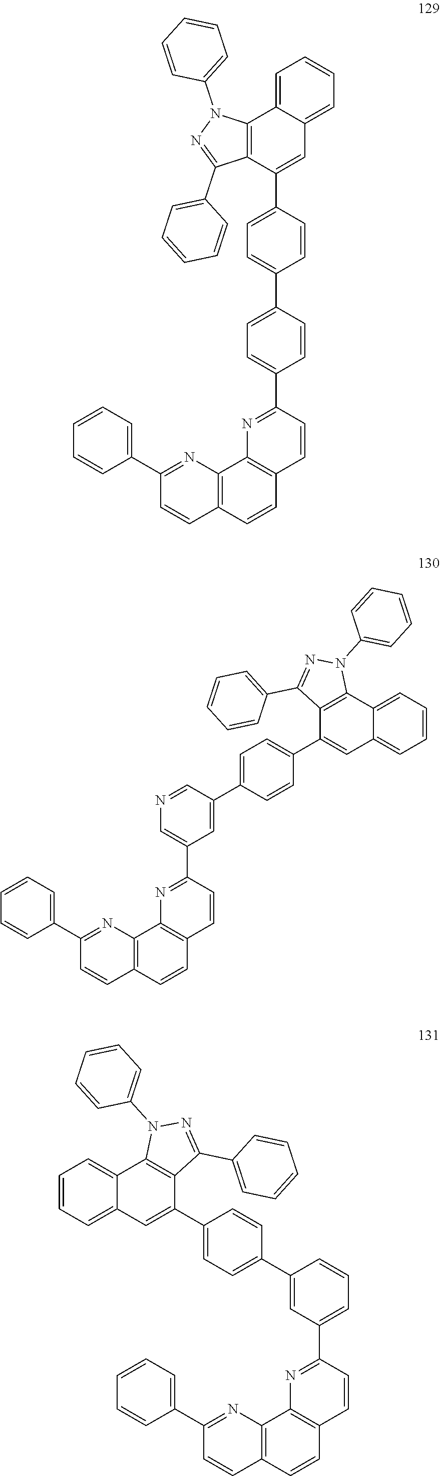 [SMILES: c1ccc(-c2ccc3ccc4ccc(-c5ccc(-c6ccc(-c7cc8ccccc8c8c7c(-c7ccccc7)nn8-c7ccccc7)cc6)cc5)nc4c3n2)cc1 ; c1ccc(-c2ccc3ccc4ccc(-c5cncc(-c6ccc(-c7cc8ccccc8c8c7c(-c7ccccc7)nn8-c7ccccc7)cc6)c5)nc4c3n2)cc1 ; c1ccc(-c2ccc3ccc4ccc(-c5cccc(-c6ccc(-c7cc8ccccc8c8c7c(-c7ccccc7)nn8-c7ccccc7)cc6)c5)nc4c3n2)cc1]

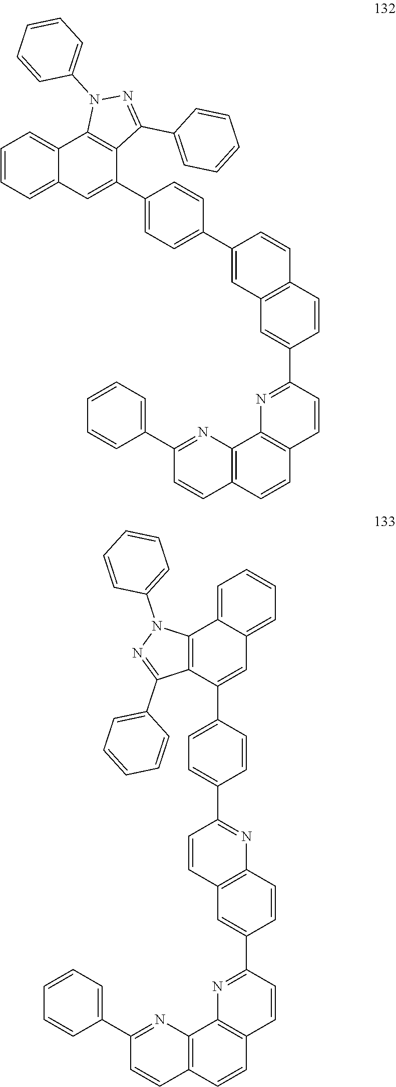 [SMILES: c1ccc(-c2ccc3ccc4ccc(-c5ccc6ccc(-c7ccc(-c8cc9ccccc9c9c8c(-c8ccccc8)nn9-c8ccccc8)cc7)cc6c5)nc4c3n2)cc1 ; c1ccc(-c2ccc3ccc4ccc(-c5ccc6nc(-c7ccc(-c8cc9ccccc9c9c8c(-c8ccccc8)nn9-c8ccccc8)cc7)ccc6c5)nc4c3n2)cc1]

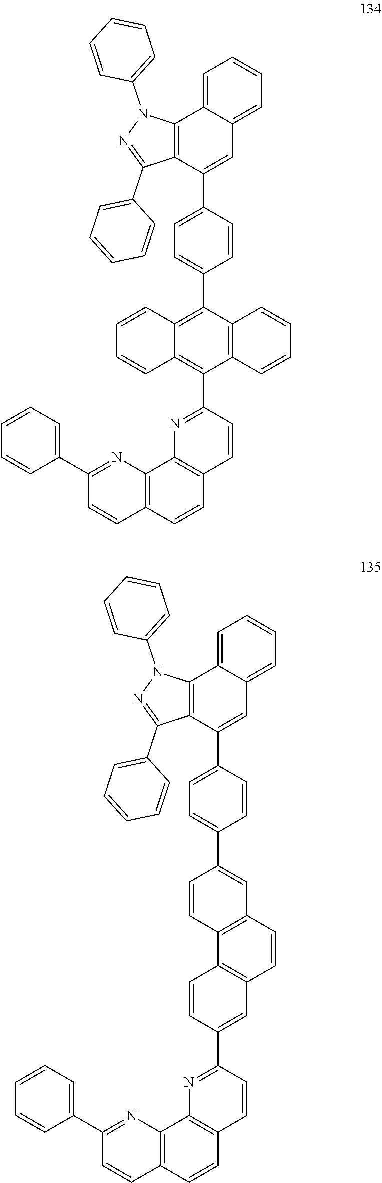 [SMILES: c1ccc(-c2ccc3ccc4ccc(-c5c6ccccc6c(-c6ccc(-c7cc8ccccc8c8c7c(-c7ccccc7)nn8-c7ccccc7)cc6)c6ccccc56)nc4c3n2)cc1 ; c1ccc(-c2ccc3ccc4ccc(-c5ccc6c(ccc7cc(-c8ccc(-c9cc%10ccccc%10c%10c9c(-c9ccccc9)nn%10-c9ccccc9)cc8)ccc76)c5)nc4c3n2)cc1]

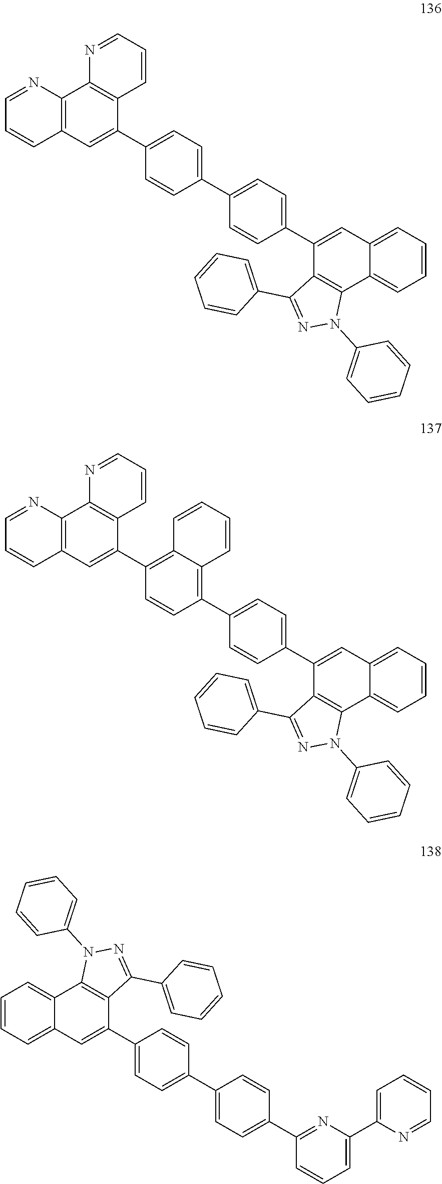 [SMILES: c1ccc(-c2nn(-c3ccccc3)c3c2c(-c2ccc(-c4ccc(-c5cc6cccnc6c6ncccc56)cc4)cc2)cc2ccccc23)cc1 ; c1ccc(-c2nn(-c3ccccc3)c3c2c(-c2ccc(-c4ccc(-c5cc6cccnc6c6ncccc56)c5ccccc45)cc2)cc2ccccc23)cc1 ; c1ccc(-c2nn(-c3ccccc3)c3c2c(-c2ccc(-c4ccc(-c5cccc(-c6ccccn6)n5)cc4)cc2)cc2ccccc23)cc1]

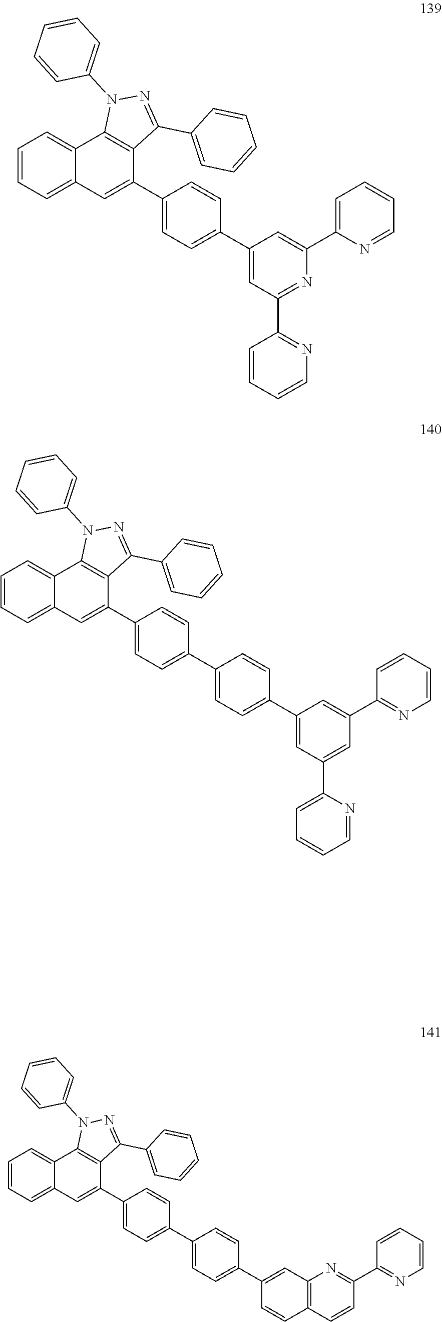 [SMILES: c1ccc(-c2nn(-c3ccccc3)c3c2c(-c2ccc(-c4cc(-c5ccccn5)nc(-c5ccccn5)c4)cc2)cc2ccccc23)cc1 ; c1ccc(-c2nn(-c3ccccc3)c3c2c(-c2ccc(-c4ccc(-c5cc(-c6ccccn6)cc(-c6ccccn6)c5)cc4)cc2)cc2ccccc23)cc1 ; c1ccc(-c2nn(-c3ccccc3)c3c2c(-c2ccc(-c4ccc(-c5ccc6ccc(-c7ccccn7)nc6c5)cc4)cc2)cc2ccccc23)cc1]

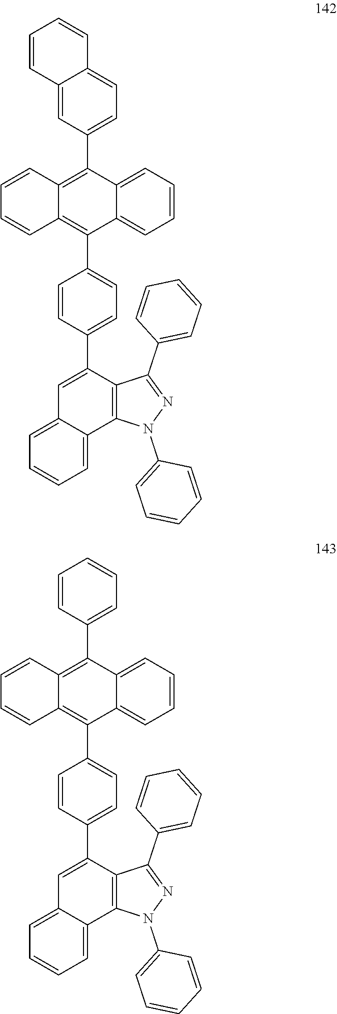 [SMILES: c1ccc(-c2nn(-c3ccccc3)c3c2c(-c2ccc(-c4c5ccccc5c(-c5ccc6ccccc6c5)c5ccccc45)cc2)cc2ccccc23)cc1 ; c1ccc(-c2c3ccccc3c(-c3ccc(-c4cc5ccccc5c5c4c(-c4ccccc4)nn5-c4ccccc4)cc3)c3ccccc23)cc1]

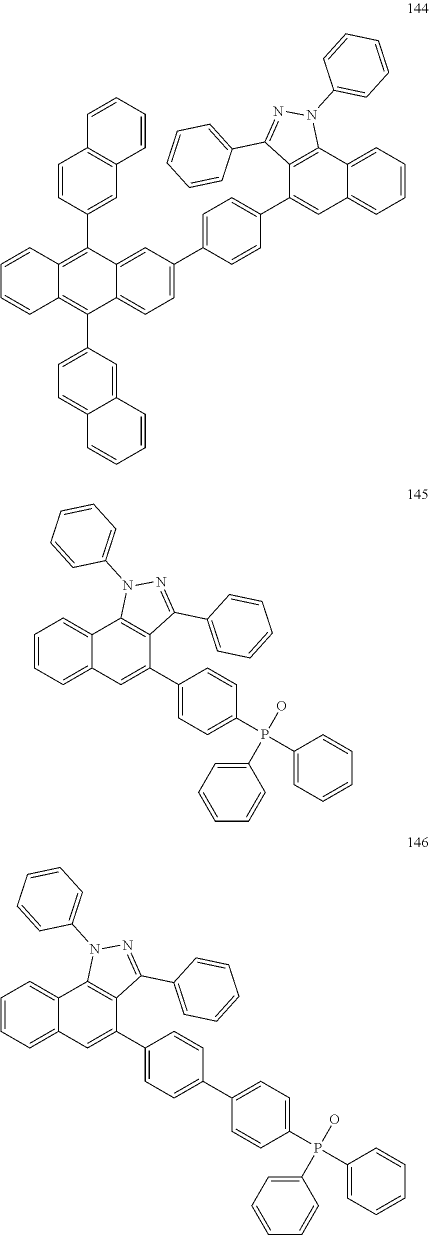

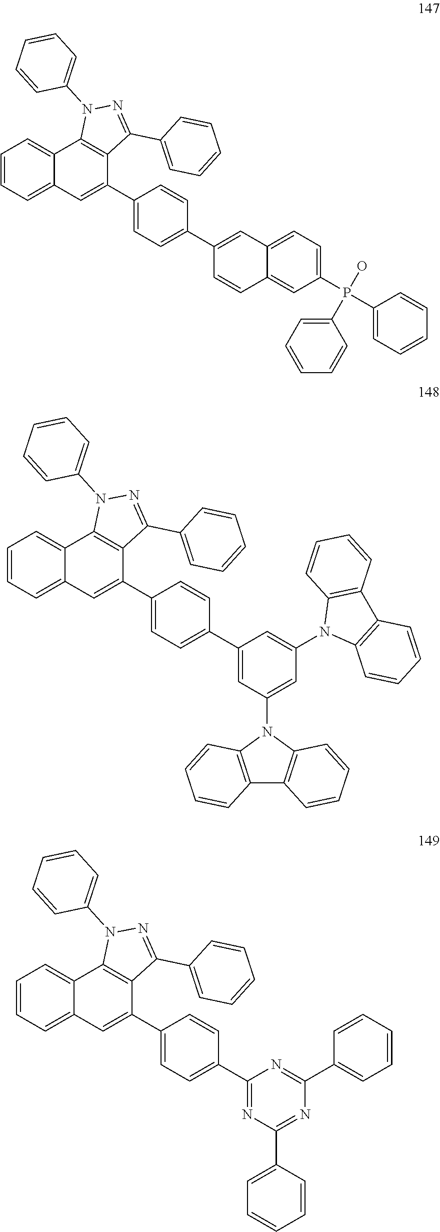

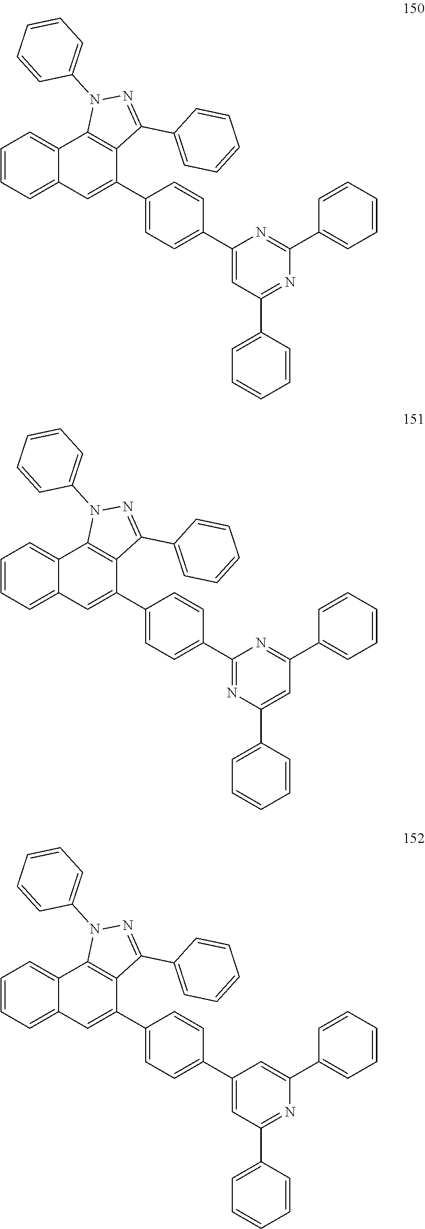 [SMILES: c1ccc(-c2cc(-c3ccc(-c4cc5ccccc5c5c4c(-c4ccccc4)nn5-c4ccccc4)cc3)nc(-c3ccccc3)n2)cc1 ; c1ccc(-c2cc(-c3ccccc3)nc(-c3ccc(-c4cc5ccccc5c5c4c(-c4ccccc4)nn5-c4ccccc4)cc3)n2)cc1 ; c1ccc(-c2cc(-c3ccc(-c4cc5ccccc5c5c4c(-c4ccccc4)nn5-c4ccccc4)cc3)cc(-c3ccccc3)n2)cc1]

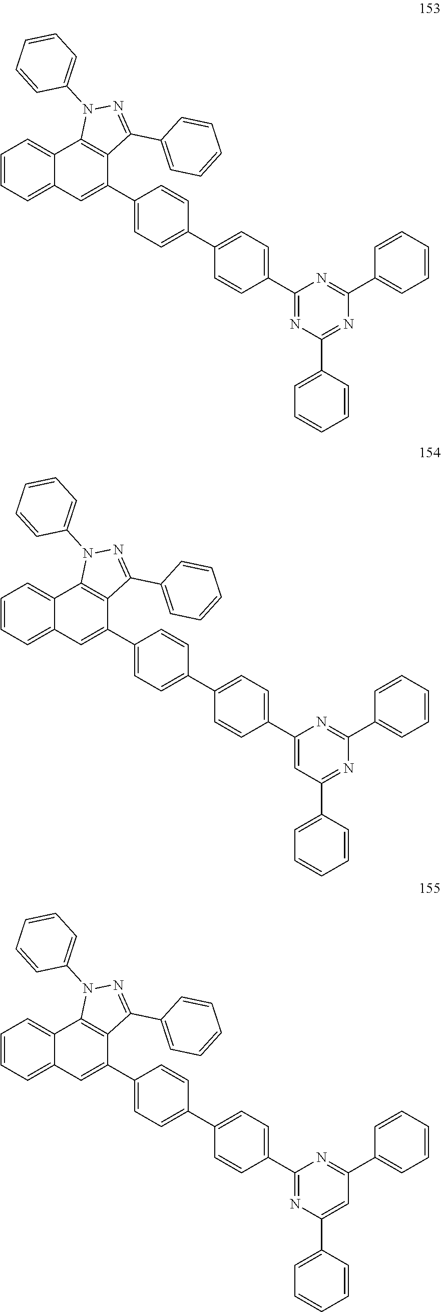 [SMILES: c1ccc(-c2nc(-c3ccccc3)nc(-c3ccc(-c4ccc(-c5cc6ccccc6c6c5c(-c5ccccc5)nn6-c5ccccc5)cc4)cc3)n2)cc1 ; c1ccc(-c2cc(-c3ccc(-c4ccc(-c5cc6ccccc6c6c5c(-c5ccccc5)nn6-c5ccccc5)cc4)cc3)nc(-c3ccccc3)n2)cc1 ; c1ccc(-c2cc(-c3ccccc3)nc(-c3ccc(-c4ccc(-c5cc6ccccc6c6c5c(-c5ccccc5)nn6-c5ccccc5)cc4)cc3)n2)cc1]

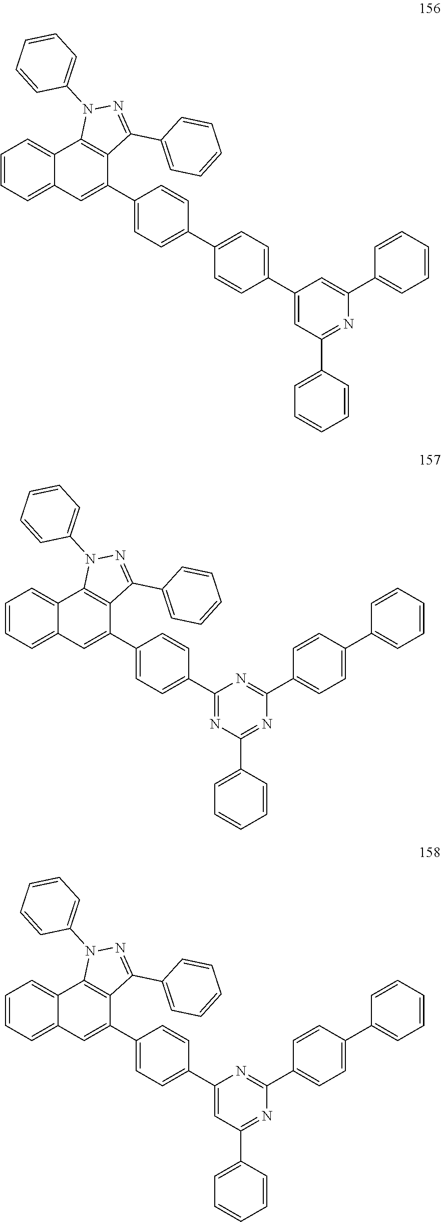 [SMILES: c1ccc(-c2cc(-c3ccc(-c4ccc(-c5cc6ccccc6c6c5c(-c5ccccc5)nn6-c5ccccc5)cc4)cc3)cc(-c3ccccc3)n2)cc1 ; c1ccc(-c2ccc(-c3nc(-c4ccccc4)nc(-c4ccc(-c5cc6ccccc6c6c5c(-c5ccccc5)nn6-c5ccccc5)cc4)n3)cc2)cc1 ; c1ccc(-c2ccc(-c3nc(-c4ccccc4)cc(-c4ccc(-c5cc6ccccc6c6c5c(-c5ccccc5)nn6-c5ccccc5)cc4)n3)cc2)cc1]

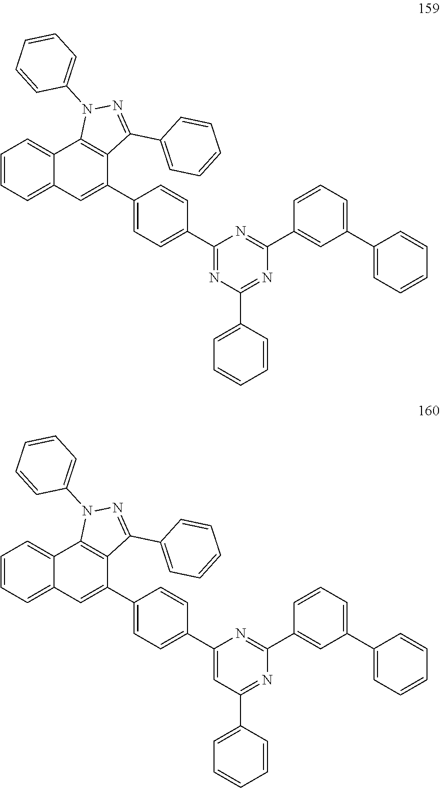 [SMILES: c1ccc(-c2cccc(-c3nc(-c4ccccc4)nc(-c4ccc(-c5cc6ccccc6c6c5c(-c5ccccc5)nn6-c5ccccc5)cc4)n3)c2)cc1 ; c1ccc(-c2cccc(-c3nc(-c4ccccc4)cc(-c4ccc(-c5cc6ccccc6c6c5c(-c5ccccc5)nn6-c5ccccc5)cc4)n3)c2)cc1]

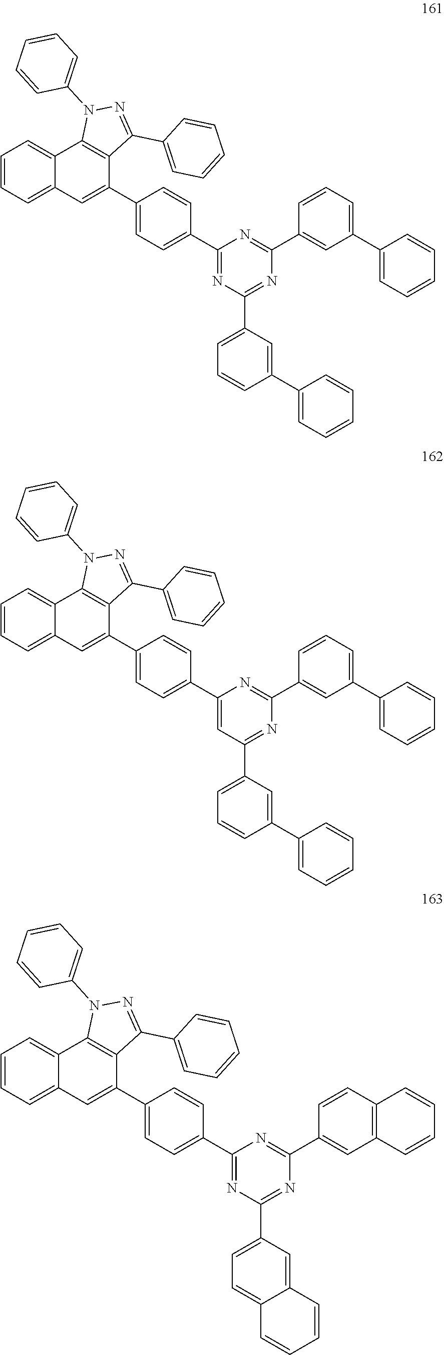 [SMILES: c1ccc(-c2cccc(-c3nc(-c4ccc(-c5cc6ccccc6c6c5c(-c5ccccc5)nn6-c5ccccc5)cc4)nc(-c4cccc(-c5ccccc5)c4)n3)c2)cc1 ; c1ccc(-c2cccc(-c3cc(-c4ccc(-c5cc6ccccc6c6c5c(-c5ccccc5)nn6-c5ccccc5)cc4)nc(-c4cccc(-c5ccccc5)c4)n3)c2)cc1 ; c1ccc(-c2nn(-c3ccccc3)c3c2c(-c2ccc(-c4nc(-c5ccc6ccccc6c5)nc(-c5ccc6ccccc6c5)n4)cc2)cc2ccccc23)cc1]

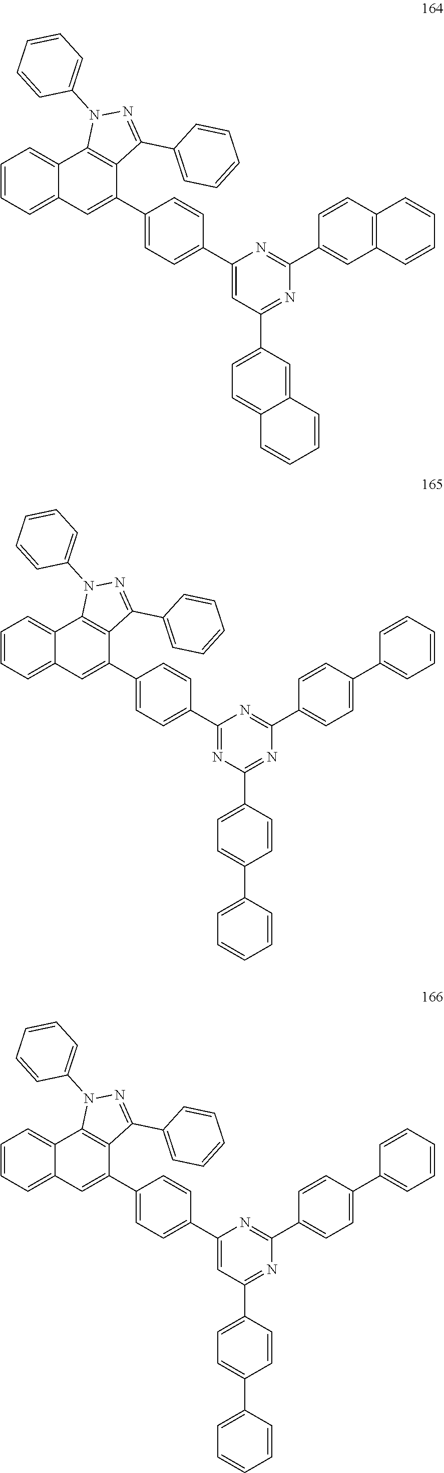 [SMILES: c1ccc(-c2nn(-c3ccccc3)c3c2c(-c2ccc(-c4cc(-c5ccc6ccccc6c5)nc(-c5ccc6ccccc6c5)n4)cc2)cc2ccccc23)cc1 ; c1ccc(-c2ccc(-c3nc(-c4ccc(-c5ccccc5)cc4)nc(-c4ccc(-c5cc6ccccc6c6c5c(-c5ccccc5)nn6-c5ccccc5)cc4)n3)cc2)cc1 ; c1ccc(-c2ccc(-c3cc(-c4ccc(-c5cc6ccccc6c6c5c(-c5ccccc5)nn6-c5ccccc5)cc4)nc(-c4ccc(-c5ccccc5)cc4)n3)cc2)cc1]

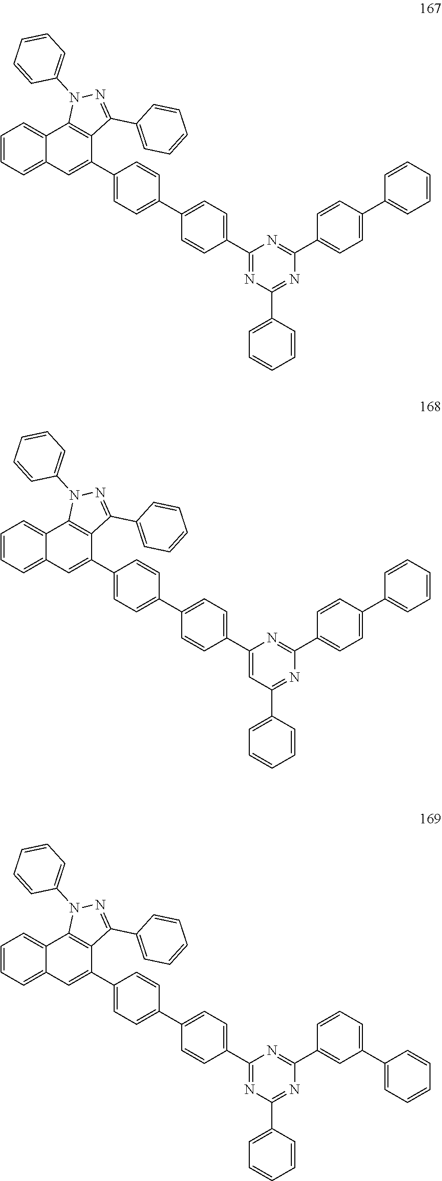 [SMILES: c1ccc(-c2ccc(-c3nc(-c4ccccc4)nc(-c4ccc(-c5ccc(-c6cc7ccccc7c7c6c(-c6ccccc6)nn7-c6ccccc6)cc5)cc4)n3)cc2)cc1 ; c1ccc(-c2ccc(-c3nc(-c4ccccc4)cc(-c4ccc(-c5ccc(-c6cc7ccccc7c7c6c(-c6ccccc6)nn7-c6ccccc6)cc5)cc4)n3)cc2)cc1 ; c1ccc(-c2cccc(-c3nc(-c4ccccc4)nc(-c4ccc(-c5ccc(-c6cc7ccccc7c7c6c(-c6ccccc6)nn7-c6ccccc6)cc5)cc4)n3)c2)cc1]

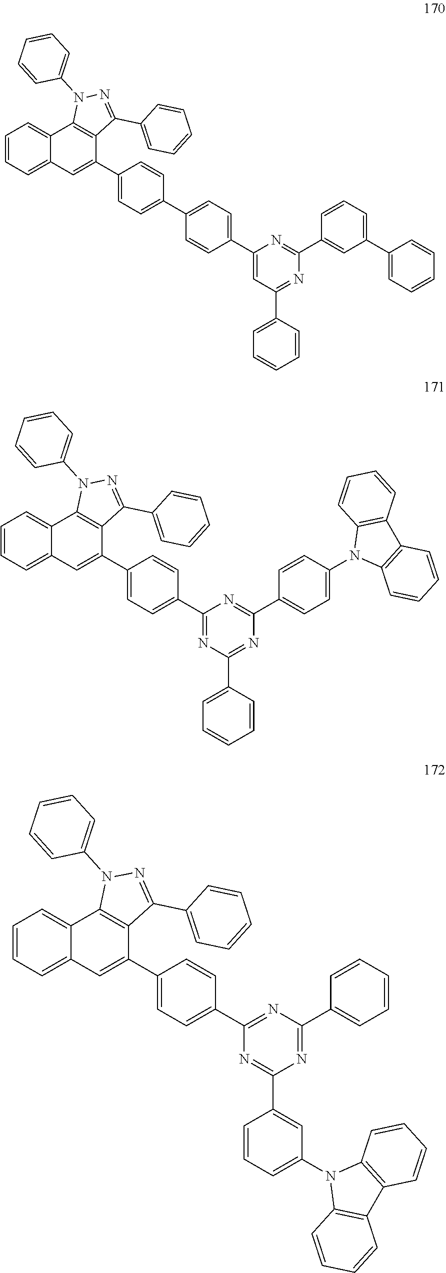 [SMILES: c1ccc(-c2cccc(-c3nc(-c4ccccc4)cc(-c4ccc(-c5ccc(-c6cc7ccccc7c7c6c(-c6ccccc6)nn7-c6ccccc6)cc5)cc4)n3)c2)cc1 ; c1ccc(-c2nc(-c3ccc(-c4cc5ccccc5c5c4c(-c4ccccc4)nn5-c4ccccc4)cc3)nc(-c3ccc(-n4c5ccccc5c5ccccc54)cc3)n2)cc1 ; c1ccc(-c2nc(-c3ccc(-c4cc5ccccc5c5c4c(-c4ccccc4)nn5-c4ccccc4)cc3)nc(-c3cccc(-n4c5ccccc5c5ccccc54)c3)n2)cc1]

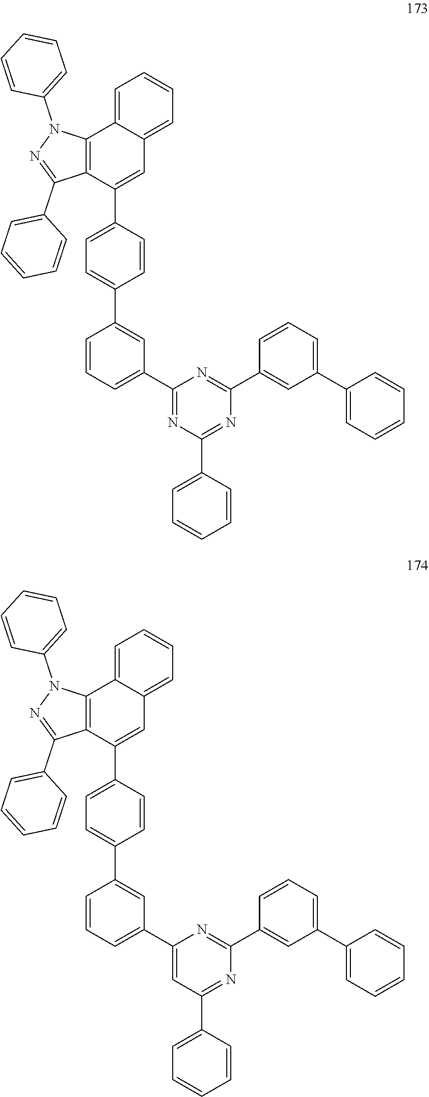 [SMILES: c1ccc(-c2cccc(-c3nc(-c4ccccc4)nc(-c4cccc(-c5ccc(-c6cc7ccccc7c7c6c(-c6ccccc6)nn7-c6ccccc6)cc5)c4)n3)c2)cc1 ; c1ccc(-c2cccc(-c3nc(-c4ccccc4)cc(-c4cccc(-c5ccc(-c6cc7ccccc7c7c6c(-c6ccccc6)nn7-c6ccccc6)cc5)c4)n3)c2)cc1]

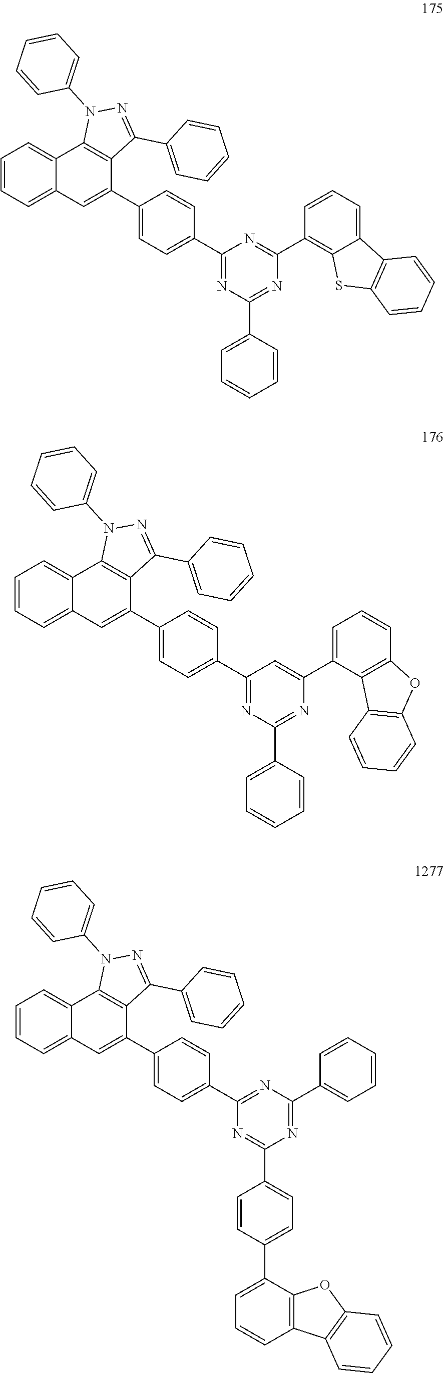 [SMILES: c1ccc(-c2nc(-c3ccc(-c4cc5ccccc5c5c4c(-c4ccccc4)nn5-c4ccccc4)cc3)nc(-c3cccc4c3sc3ccccc34)n2)cc1 ; c1ccc(-c2nc(-c3ccc(-c4cc5ccccc5c5c4c(-c4ccccc4)nn5-c4ccccc4)cc3)cc(-c3cccc4oc5ccccc5c34)n2)cc1 ; c1ccc(-c2nc(-c3ccc(-c4cccc5c4oc4ccccc45)cc3)nc(-c3ccc(-c4cc5ccccc5c5c4c(-c4ccccc4)nn5-c4ccccc4)cc3)n2)cc1]

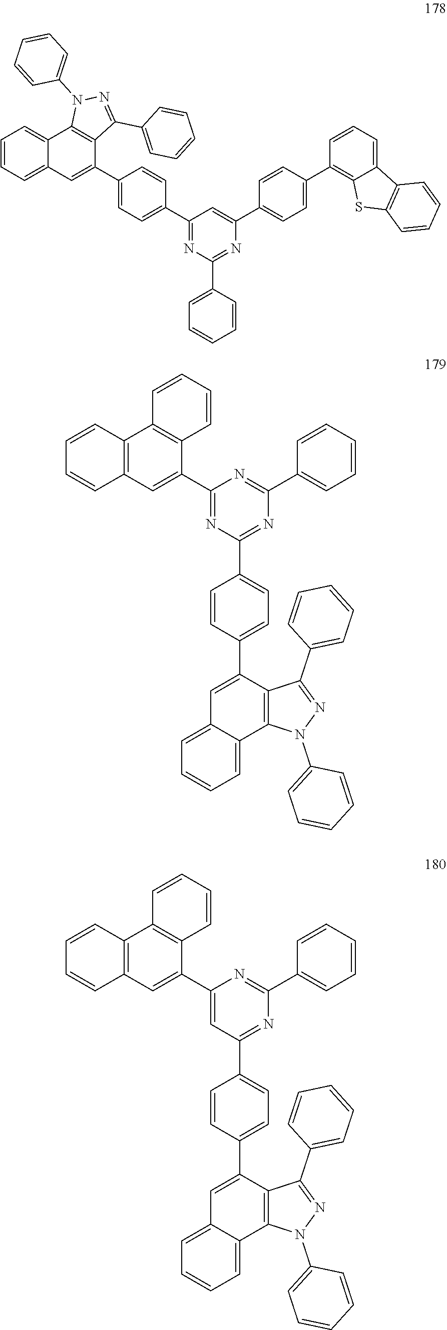

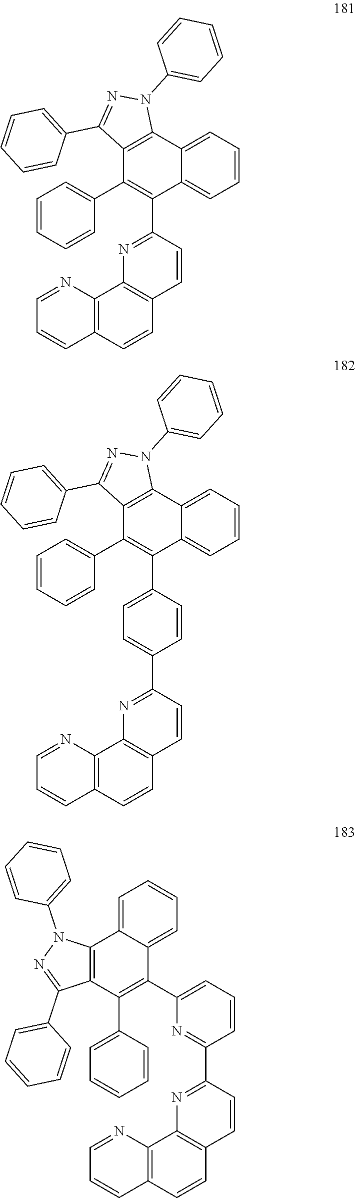

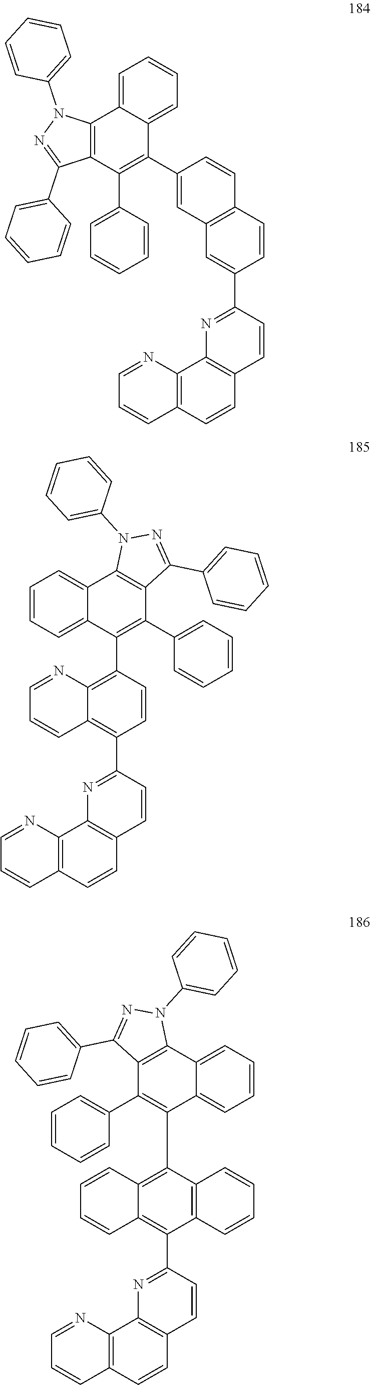 [SMILES: c1ccc(-c2nn(-c3ccccc3)c3c2c(-c2ccccc2)c(-c2ccc4ccc(-c5ccc6ccc7cccnc7c6n5)cc4c2)c2ccccc23)cc1 ; c1ccc(-c2nn(-c3ccccc3)c3c2c(-c2ccccc2)c(-c2ccc(-c4ccc5ccc6cccnc6c5n4)c4cccnc24)c2ccccc23)cc1 ; c1ccc(-c2nn(-c3ccccc3)c3c2c(-c2ccccc2)c(-c2c4ccccc4c(-c4ccc5ccc6cccnc6c5n4)c4ccccc24)c2ccccc23)cc1]

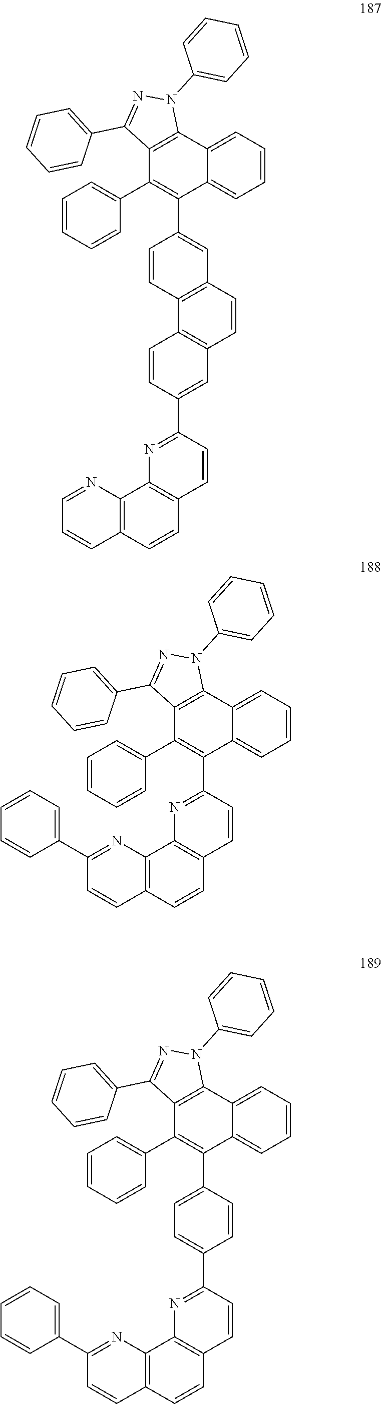 [SMILES: c1ccc(-c2nn(-c3ccccc3)c3c2c(-c2ccccc2)c(-c2ccc4c(ccc5cc(-c6ccc7ccc8cccnc8c7n6)ccc54)c2)c2ccccc23)cc1 ; c1ccc(-c2ccc3ccc4ccc(-c5c(-c6ccccc6)c6c(-c7ccccc7)nn(-c7ccccc7)c6c6ccccc56)nc4c3n2)cc1 ; c1ccc(-c2ccc3ccc4ccc(-c5ccc(-c6c(-c7ccccc7)c7c(-c8ccccc8)nn(-c8ccccc8)c7c7ccccc67)cc5)nc4c3n2)cc1]

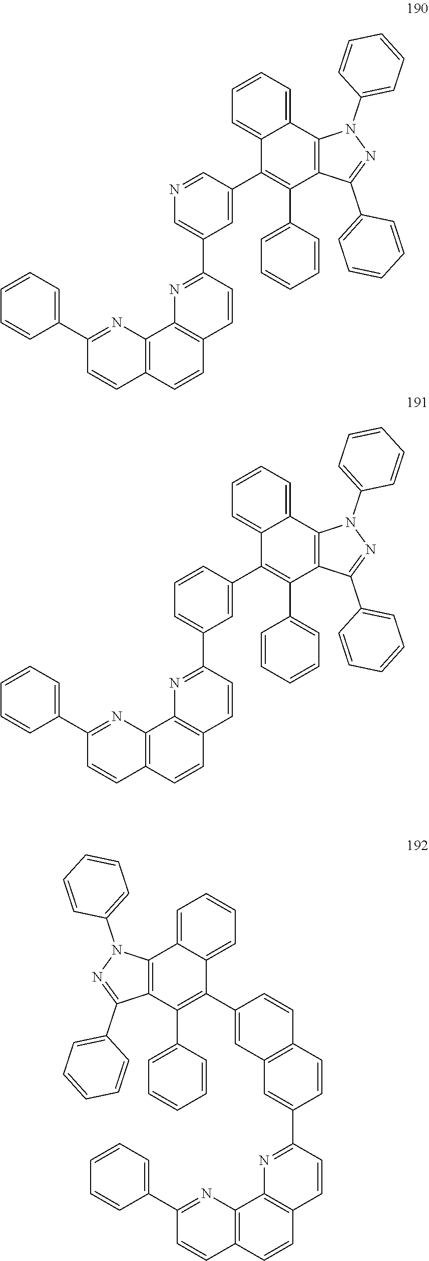 [SMILES: c1ccc(-c2ccc3ccc4ccc(-c5cncc(-c6c(-c7ccccc7)c7c(-c8ccccc8)nn(-c8ccccc8)c7c7ccccc67)c5)nc4c3n2)cc1 ; c1ccc(-c2ccc3ccc4ccc(-c5cccc(-c6c(-c7ccccc7)c7c(-c8ccccc8)nn(-c8ccccc8)c7c7ccccc67)c5)nc4c3n2)cc1 ; c1ccc(-c2ccc3ccc4ccc(-c5ccc6ccc(-c7c(-c8ccccc8)c8c(-c9ccccc9)nn(-c9ccccc9)c8c8ccccc78)cc6c5)nc4c3n2)cc1]

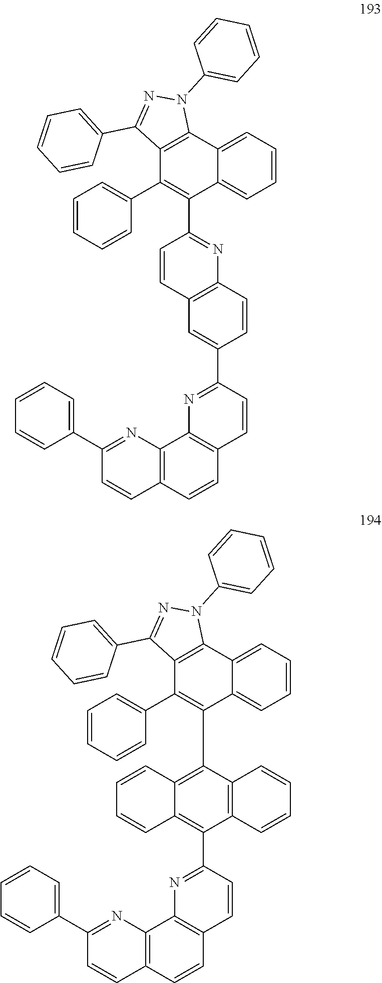 [SMILES: c1ccc(-c2ccc3ccc4ccc(-c5ccc6nc(-c7c(-c8ccccc8)c8c(-c9ccccc9)nn(-c9ccccc9)c8c8ccccc78)ccc6c5)nc4c3n2)cc1 ; c1ccc(-c2ccc3ccc4ccc(-c5c6ccccc6c(-c6c(-c7ccccc7)c7c(-c8ccccc8)nn(-c8ccccc8)c7c7ccccc67)c6ccccc56)nc4c3n2)cc1]

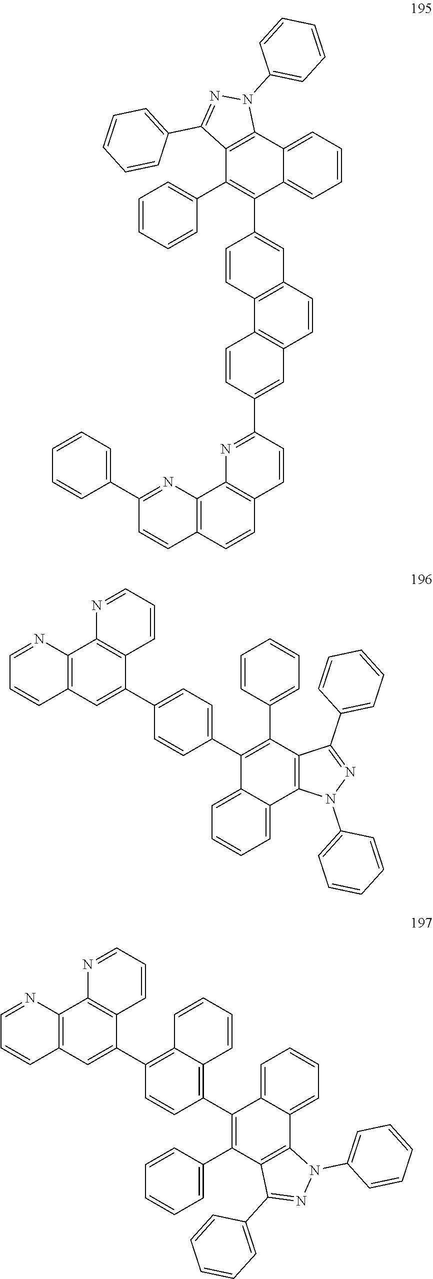 [SMILES: c1ccc(-c2ccc3ccc4ccc(-c5ccc6c(ccc7cc(-c8c(-c9ccccc9)c9c(-c%10ccccc%10)nn(-c%10ccccc%10)c9c9ccccc89)ccc76)c5)nc4c3n2)cc1 ; c1ccc(-c2nn(-c3ccccc3)c3c2c(-c2ccccc2)c(-c2ccc(-c4cc5cccnc5c5ncccc45)cc2)c2ccccc23)cc1 ; c1ccc(-c2nn(-c3ccccc3)c3c2c(-c2ccccc2)c(-c2ccc(-c4cc5cccnc5c5ncccc45)c4ccccc24)c2ccccc23)cc1]

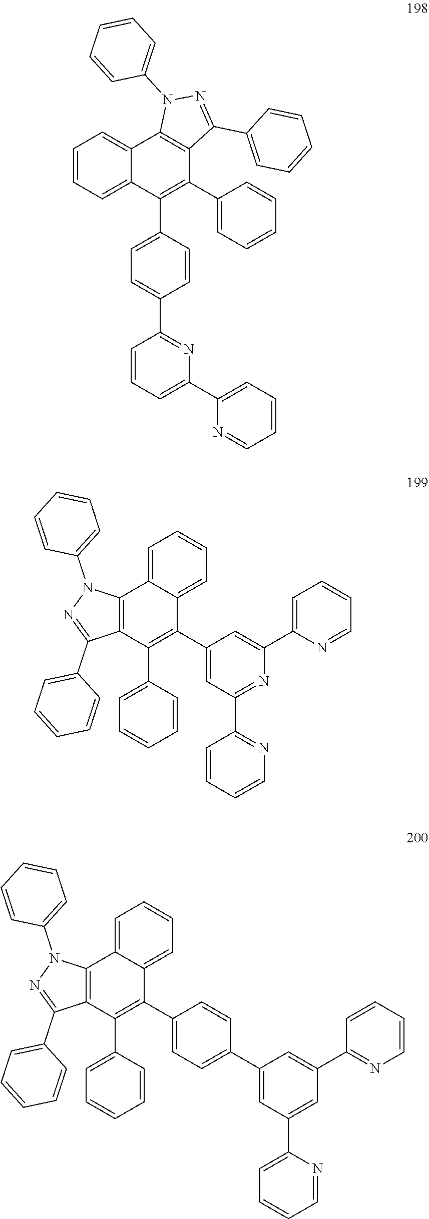 [SMILES: c1ccc(-c2nn(-c3ccccc3)c3c2c(-c2ccccc2)c(-c2ccc(-c4cccc(-c5ccccn5)n4)cc2)c2ccccc23)cc1 ; c1ccc(-c2nn(-c3ccccc3)c3c2c(-c2ccccc2)c(-c2cc(-c4ccccn4)nc(-c4ccccn4)c2)c2ccccc23)cc1 ; c1ccc(-c2nn(-c3ccccc3)c3c2c(-c2ccccc2)c(-c2ccc(-c4cc(-c5ccccn5)cc(-c5ccccn5)c4)cc2)c2ccccc23)cc1]

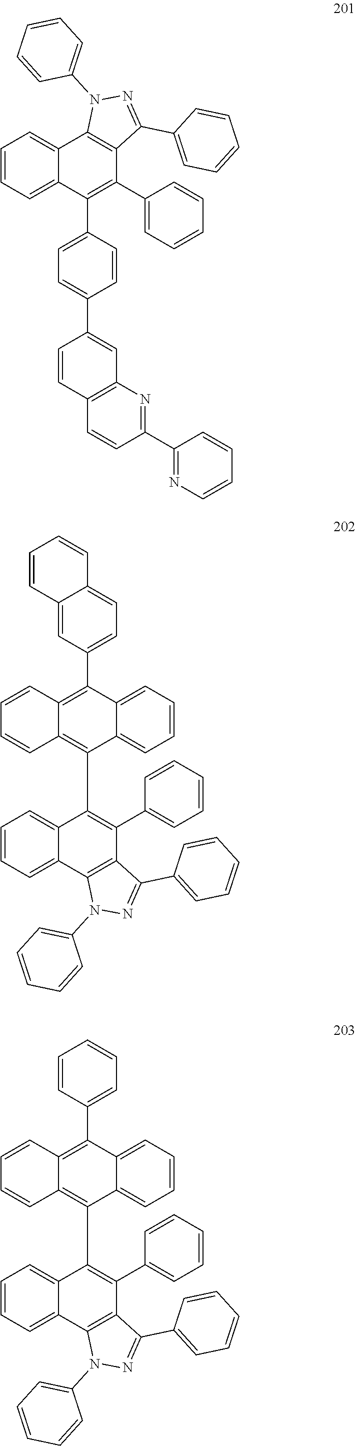 [SMILES: c1ccc(-c2nn(-c3ccccc3)c3c2c(-c2ccccc2)c(-c2ccc(-c4ccc5ccc(-c6ccccn6)nc5c4)cc2)c2ccccc23)cc1 ; c1ccc(-c2nn(-c3ccccc3)c3c2c(-c2ccccc2)c(-c2c4ccccc4c(-c4ccc5ccccc5c4)c4ccccc24)c2ccccc23)cc1 ; c1ccc(-c2c3ccccc3c(-c3c(-c4ccccc4)c4c(-c5ccccc5)nn(-c5ccccc5)c4c4ccccc34)c3ccccc23)cc1]

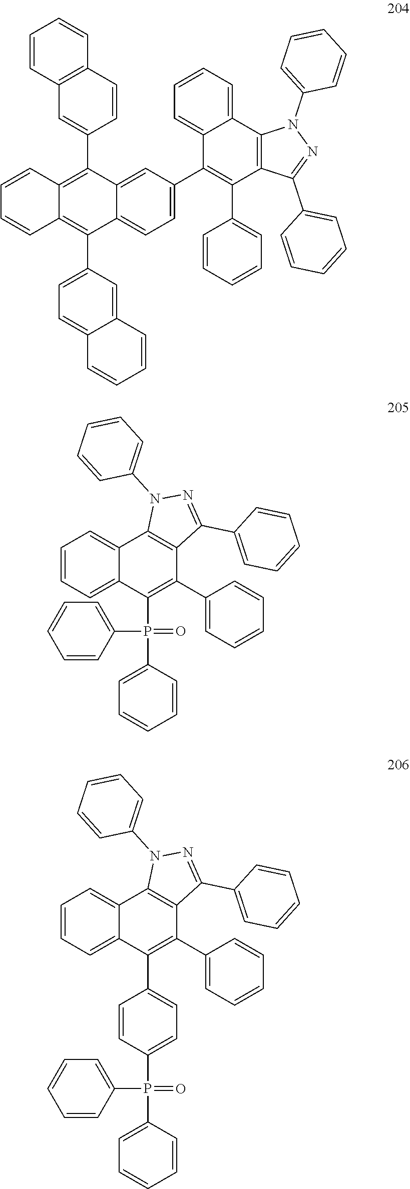

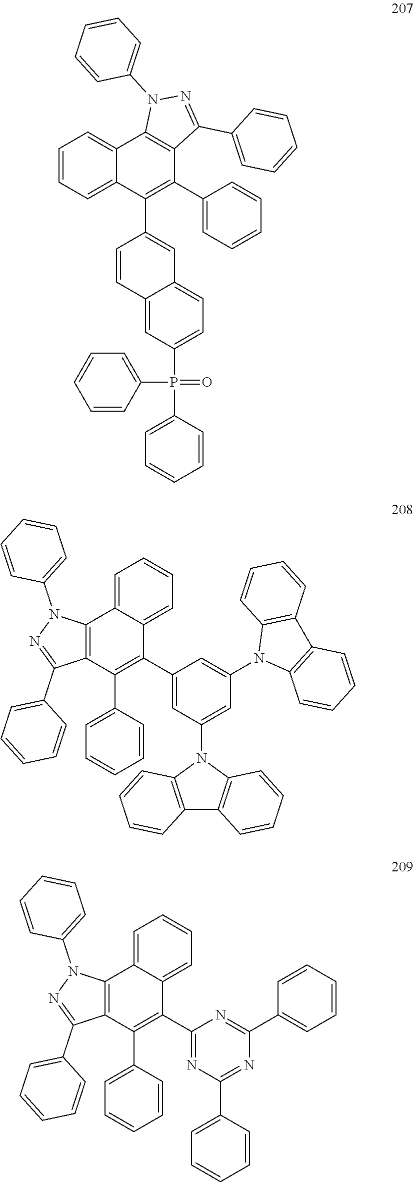 [SMILES: O=P(c1ccccc1)(c1ccccc1)c1ccc2cc(-c3c(-c4ccccc4)c4c(-c5ccccc5)nn(-c5ccccc5)c4c4ccccc34)ccc2c1 ; c1ccc(-c2nn(-c3ccccc3)c3c2c(-c2ccccc2)c(-c2cc(-n4c5ccccc5c5ccccc54)cc(-n4c5ccccc5c5ccccc54)c2)c2ccccc23)cc1 ; c1ccc(-c2nc(-c3ccccc3)nc(-c3c(-c4ccccc4)c4c(-c5ccccc5)nn(-c5ccccc5)c4c4ccccc34)n2)cc1]

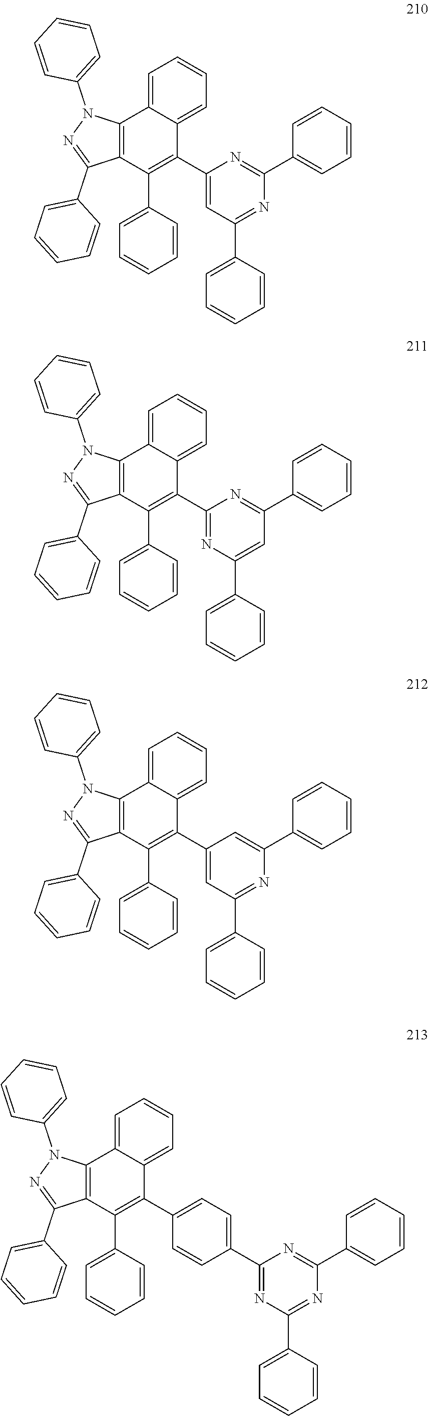 [SMILES: c1ccc(-c2cc(-c3c(-c4ccccc4)c4c(-c5ccccc5)nn(-c5ccccc5)c4c4ccccc34)nc(-c3ccccc3)n2)cc1 ; c1ccc(-c2cc(-c3ccccc3)nc(-c3c(-c4ccccc4)c4c(-c5ccccc5)nn(-c5ccccc5)c4c4ccccc34)n2)cc1 ; c1ccc(-c2cc(-c3c(-c4ccccc4)c4c(-c5ccccc5)nn(-c5ccccc5)c4c4ccccc34)cc(-c3ccccc3)n2)cc1 ; c1ccc(-c2nc(-c3ccccc3)nc(-c3ccc(-c4c(-c5ccccc5)c5c(-c6ccccc6)nn(-c6ccccc6)c5c5ccccc45)cc3)n2)cc1]

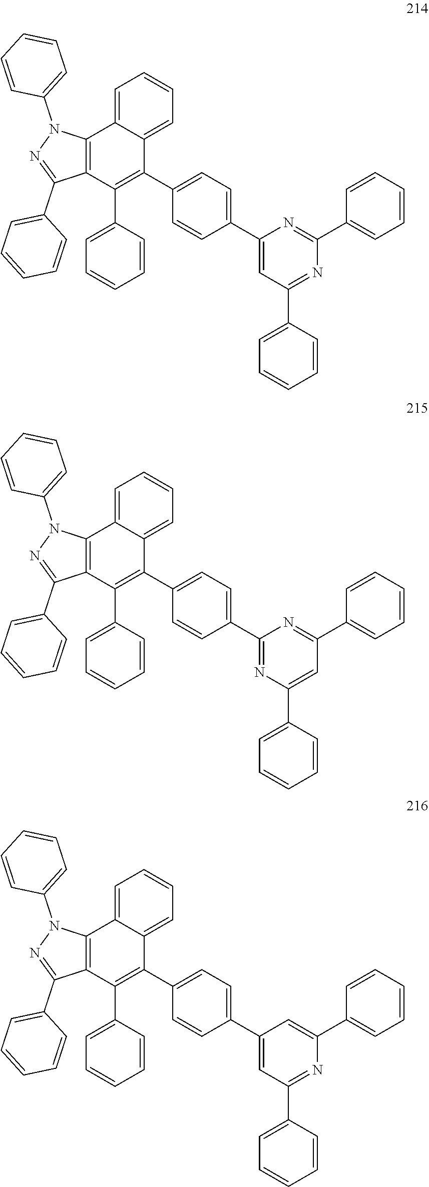 [SMILES: c1ccc(-c2cc(-c3ccc(-c4c(-c5ccccc5)c5c(-c6ccccc6)nn(-c6ccccc6)c5c5ccccc45)cc3)nc(-c3ccccc3)n2)cc1 ; c1ccc(-c2cc(-c3ccccc3)nc(-c3ccc(-c4c(-c5ccccc5)c5c(-c6ccccc6)nn(-c6ccccc6)c5c5ccccc45)cc3)n2)cc1 ; c1ccc(-c2cc(-c3ccc(-c4c(-c5ccccc5)c5c(-c6ccccc6)nn(-c6ccccc6)c5c5ccccc45)cc3)cc(-c3ccccc3)n2)cc1]

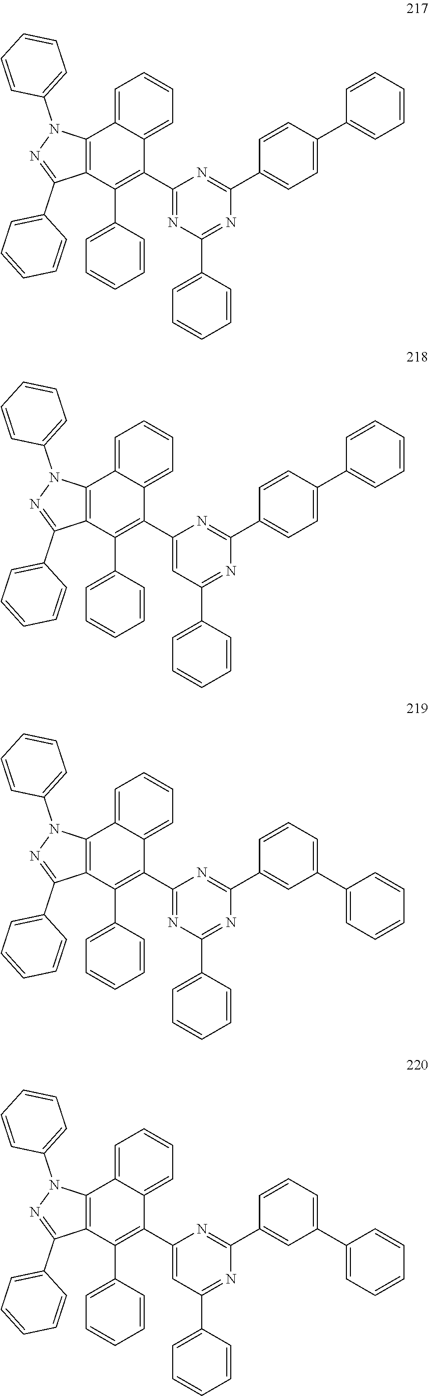 [SMILES: c1ccc(-c2ccc(-c3nc(-c4ccccc4)nc(-c4c(-c5ccccc5)c5c(-c6ccccc6)nn(-c6ccccc6)c5c5ccccc45)n3)cc2)cc1 ; c1ccc(-c2ccc(-c3nc(-c4ccccc4)cc(-c4c(-c5ccccc5)c5c(-c6ccccc6)nn(-c6ccccc6)c5c5ccccc45)n3)cc2)cc1 ; c1ccc(-c2cccc(-c3nc(-c4ccccc4)nc(-c4c(-c5ccccc5)c5c(-c6ccccc6)nn(-c6ccccc6)c5c5ccccc45)n3)c2)cc1 ; c1ccc(-c2cccc(-c3nc(-c4ccccc4)cc(-c4c(-c5ccccc5)c5c(-c6ccccc6)nn(-c6ccccc6)c5c5ccccc45)n3)c2)cc1]

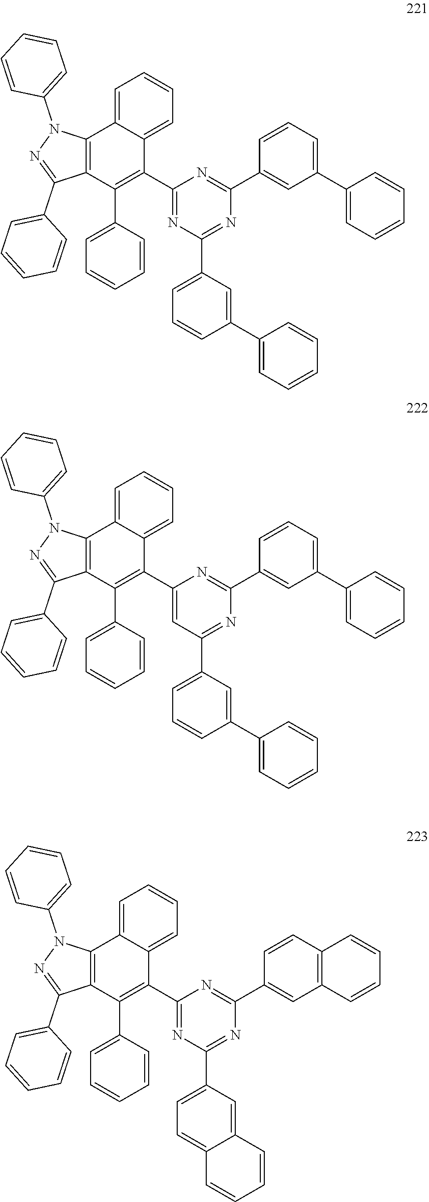 [SMILES: c1ccc(-c2cccc(-c3nc(-c4cccc(-c5ccccc5)c4)nc(-c4c(-c5ccccc5)c5c(-c6ccccc6)nn(-c6ccccc6)c5c5ccccc45)n3)c2)cc1 ; c1ccc(-c2cccc(-c3cc(-c4c(-c5ccccc5)c5c(-c6ccccc6)nn(-c6ccccc6)c5c5ccccc45)nc(-c4cccc(-c5ccccc5)c4)n3)c2)cc1 ; c1ccc(-c2nn(-c3ccccc3)c3c2c(-c2ccccc2)c(-c2nc(-c4ccc5ccccc5c4)nc(-c4ccc5ccccc5c4)n2)c2ccccc23)cc1]

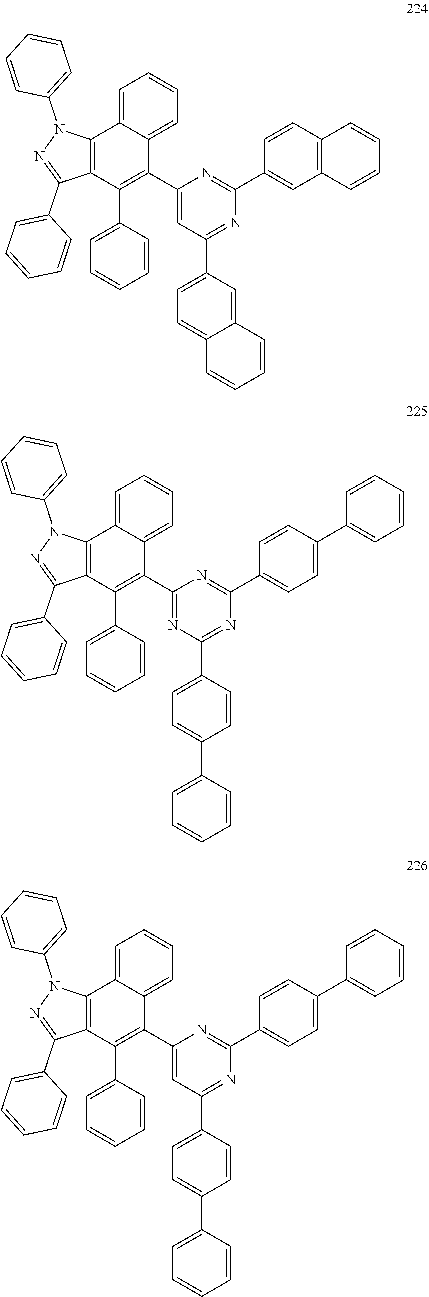 [SMILES: c1ccc(-c2nn(-c3ccccc3)c3c2c(-c2ccccc2)c(-c2cc(-c4ccc5ccccc5c4)nc(-c4ccc5ccccc5c4)n2)c2ccccc23)cc1 ; c1ccc(-c2ccc(-c3nc(-c4ccc(-c5ccccc5)cc4)nc(-c4c(-c5ccccc5)c5c(-c6ccccc6)nn(-c6ccccc6)c5c5ccccc45)n3)cc2)cc1 ; c1ccc(-c2ccc(-c3cc(-c4c(-c5ccccc5)c5c(-c6ccccc6)nn(-c6ccccc6)c5c5ccccc45)nc(-c4ccc(-c5ccccc5)cc4)n3)cc2)cc1]

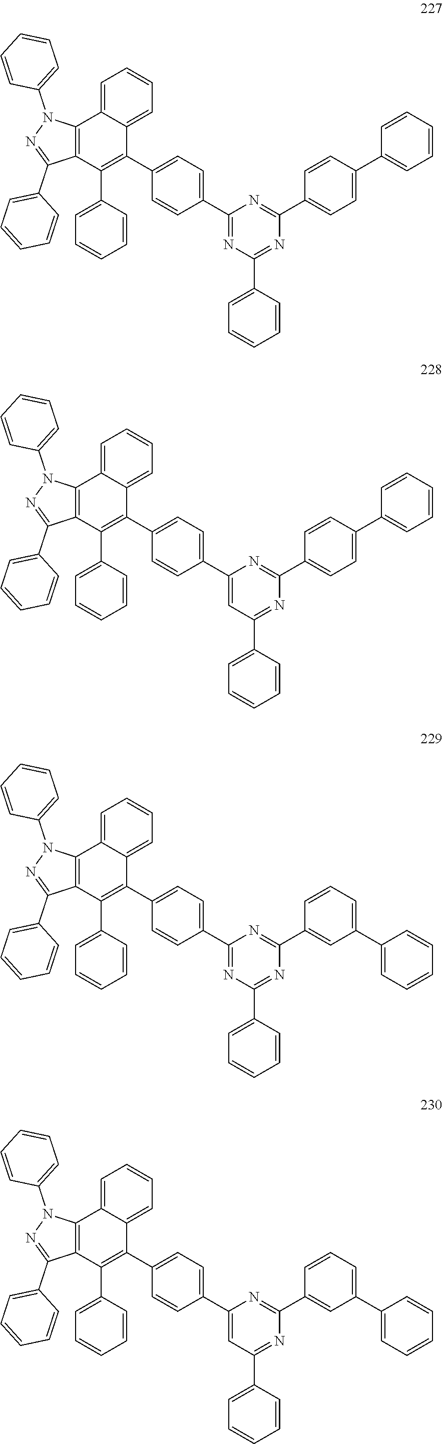 [SMILES: c1ccc(-c2ccc(-c3nc(-c4ccccc4)nc(-c4ccc(-c5c(-c6ccccc6)c6c(-c7ccccc7)nn(-c7ccccc7)c6c6ccccc56)cc4)n3)cc2)cc1 ; c1ccc(-c2ccc(-c3nc(-c4ccccc4)cc(-c4ccc(-c5c(-c6ccccc6)c6c(-c7ccccc7)nn(-c7ccccc7)c6c6ccccc56)cc4)n3)cc2)cc1 ; c1ccc(-c2cccc(-c3nc(-c4ccccc4)nc(-c4ccc(-c5c(-c6ccccc6)c6c(-c7ccccc7)nn(-c7ccccc7)c6c6ccccc56)cc4)n3)c2)cc1 ; c1ccc(-c2cccc(-c3nc(-c4ccccc4)cc(-c4ccc(-c5c(-c6ccccc6)c6c(-c7ccccc7)nn(-c7ccccc7)c6c6ccccc56)cc4)n3)c2)cc1]

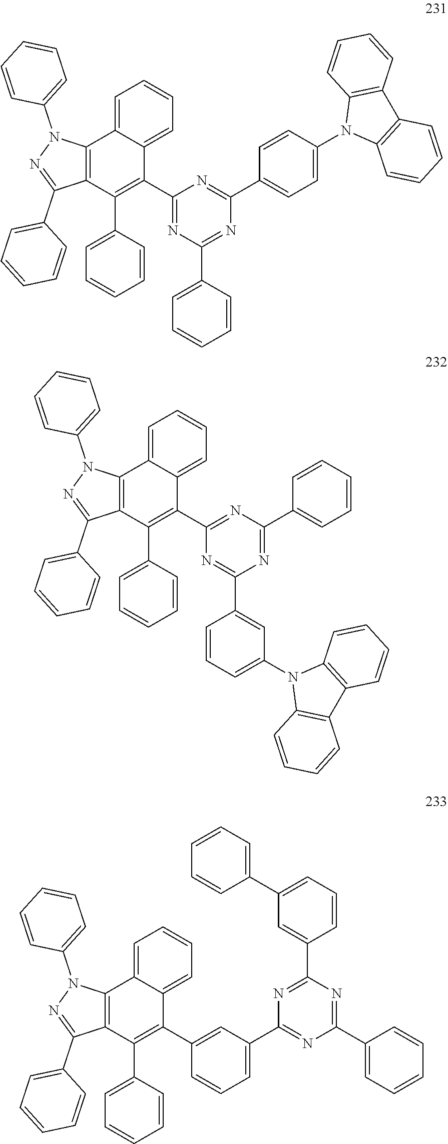 [SMILES: c1ccc(-c2nc(-c3ccc(-n4c5ccccc5c5ccccc54)cc3)nc(-c3c(-c4ccccc4)c4c(-c5ccccc5)nn(-c5ccccc5)c4c4ccccc34)n2)cc1 ; c1ccc(-c2nc(-c3cccc(-n4c5ccccc5c5ccccc54)c3)nc(-c3c(-c4ccccc4)c4c(-c5ccccc5)nn(-c5ccccc5)c4c4ccccc34)n2)cc1 ; c1ccc(-c2cccc(-c3nc(-c4ccccc4)nc(-c4cccc(-c5c(-c6ccccc6)c6c(-c7ccccc7)nn(-c7ccccc7)c6c6ccccc56)c4)n3)c2)cc1]

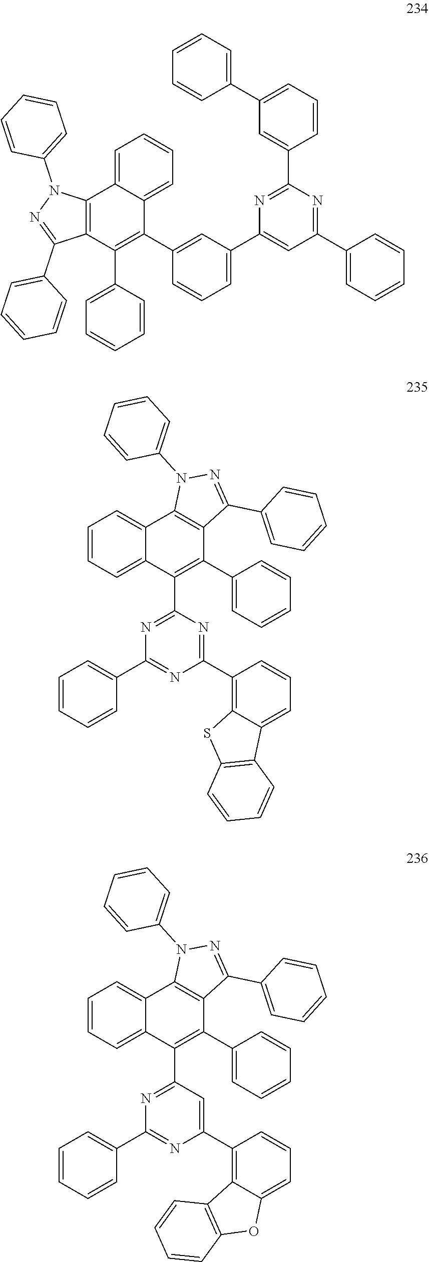 [SMILES: c1ccc(-c2cccc(-c3nc(-c4ccccc4)cc(-c4cccc(-c5c(-c6ccccc6)c6c(-c7ccccc7)nn(-c7ccccc7)c6c6ccccc56)c4)n3)c2)cc1 ; c1ccc(-c2nc(-c3c(-c4ccccc4)c4c(-c5ccccc5)nn(-c5ccccc5)c4c4ccccc34)nc(-c3cccc4c3sc3ccccc34)n2)cc1 ; c1ccc(-c2nc(-c3c(-c4ccccc4)c4c(-c5ccccc5)nn(-c5ccccc5)c4c4ccccc34)cc(-c3cccc4oc5ccccc5c34)n2)cc1]

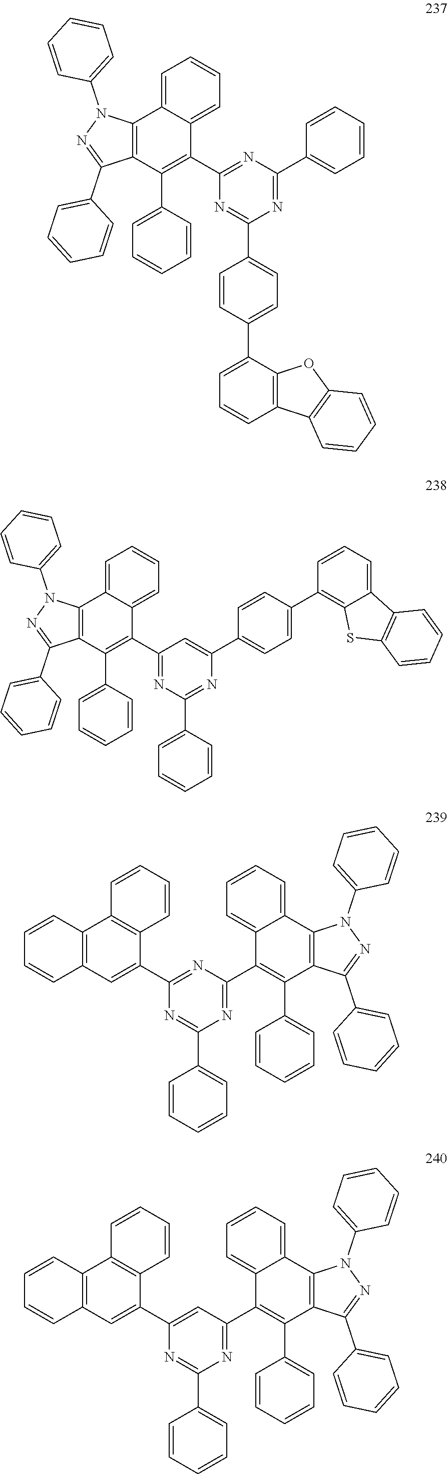 [SMILES: c1ccc(-c2nc(-c3ccc(-c4cccc5c4oc4ccccc45)cc3)nc(-c3c(-c4ccccc4)c4c(-c5ccccc5)nn(-c5ccccc5)c4c4ccccc34)n2)cc1 ; c1ccc(-c2nc(-c3ccc(-c4cccc5c4sc4ccccc45)cc3)cc(-c3c(-c4ccccc4)c4c(-c5ccccc5)nn(-c5ccccc5)c4c4ccccc34)n2)cc1 ; c1ccc(-c2nc(-c3cc4ccccc4c4ccccc34)nc(-c3c(-c4ccccc4)c4c(-c5ccccc5)nn(-c5ccccc5)c4c4ccccc34)n2)cc1 ; c1ccc(-c2nc(-c3cc4ccccc4c4ccccc34)cc(-c3c(-c4ccccc4)c4c(-c5ccccc5)nn(-c5ccccc5)c4c4ccccc34)n2)cc1]

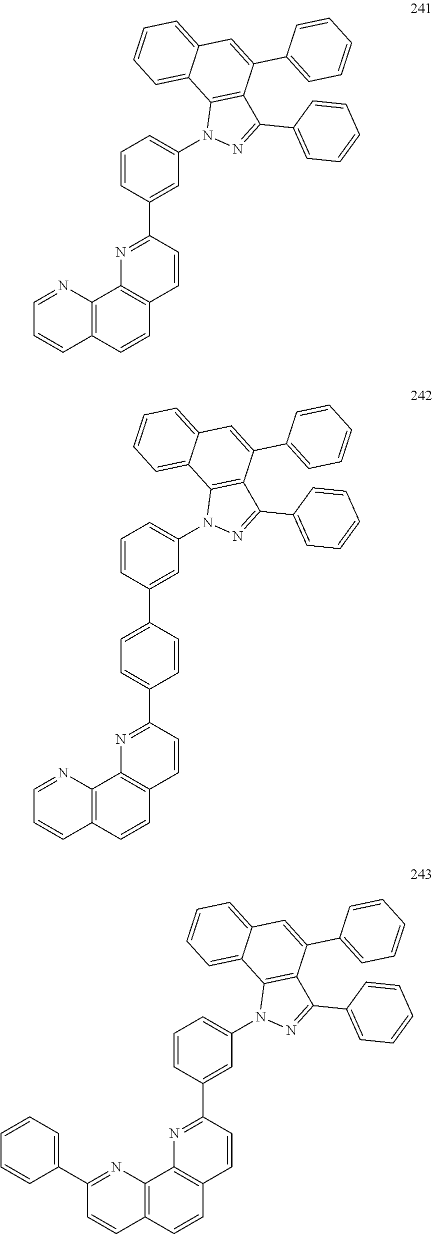 [SMILES: c1ccc(-c2cc3ccccc3c3c2c(-c2ccccc2)nn3-c2cccc(-c3ccc4ccc5cccnc5c4n3)c2)cc1 ; c1ccc(-c2cc3ccccc3c3c2c(-c2ccccc2)nn3-c2cccc(-c3ccc(-c4ccc5ccc6cccnc6c5n4)cc3)c2)cc1 ; c1ccc(-c2ccc3ccc4ccc(-c5cccc(-n6nc(-c7ccccc7)c7c(-c8ccccc8)cc8ccccc8c76)c5)nc4c3n2)cc1]

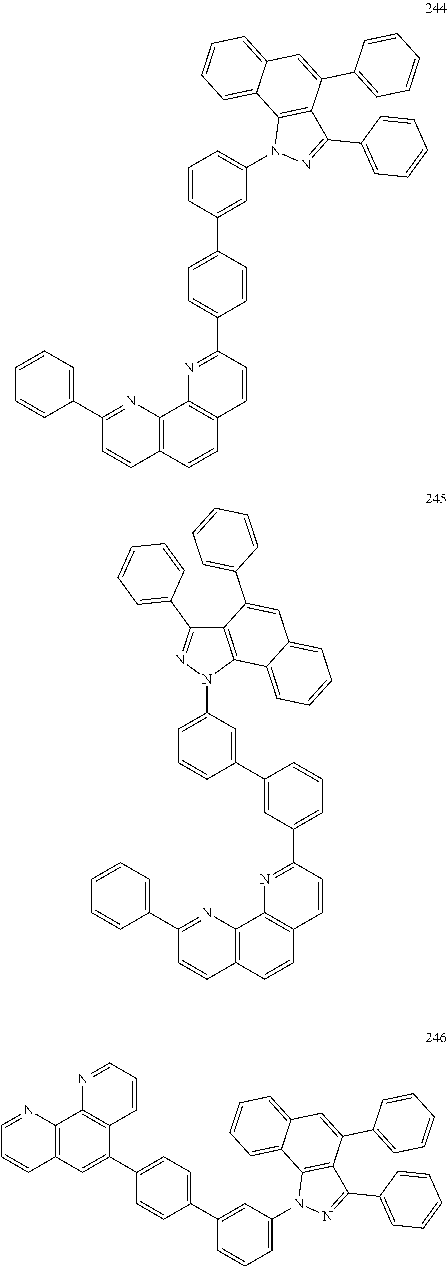 [SMILES: c1ccc(-c2ccc3ccc4ccc(-c5ccc(-c6cccc(-n7nc(-c8ccccc8)c8c(-c9ccccc9)cc9ccccc9c87)c6)cc5)nc4c3n2)cc1 ; c1ccc(-c2ccc3ccc4ccc(-c5cccc(-c6cccc(-n7nc(-c8ccccc8)c8c(-c9ccccc9)cc9ccccc9c87)c6)c5)nc4c3n2)cc1 ; c1ccc(-c2cc3ccccc3c3c2c(-c2ccccc2)nn3-c2cccc(-c3ccc(-c4cc5cccnc5c5ncccc45)cc3)c2)cc1]

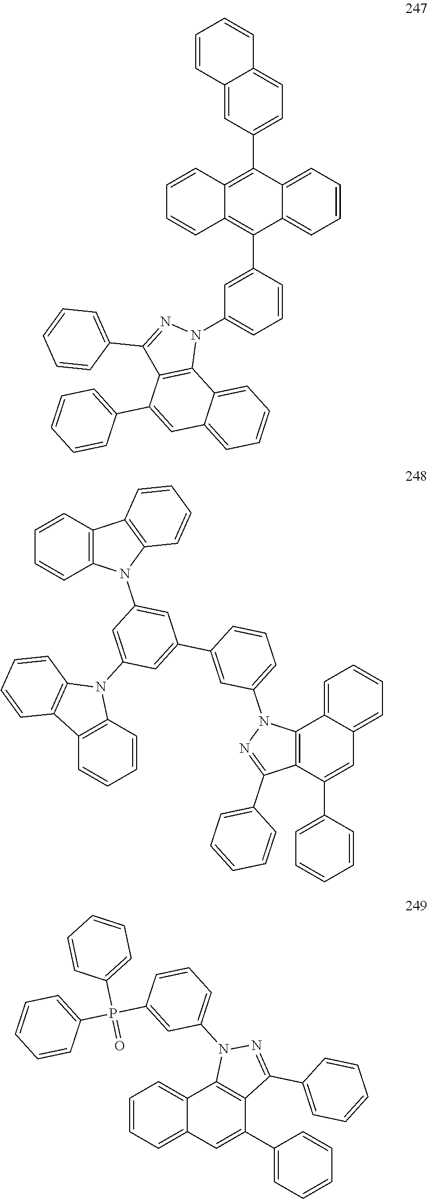 [SMILES: c1ccc(-c2cc3ccccc3c3c2c(-c2ccccc2)nn3-c2cccc(-c3c4ccccc4c(-c4ccc5ccccc5c4)c4ccccc34)c2)cc1 ; c1ccc(-c2cc3ccccc3c3c2c(-c2ccccc2)nn3-c2cccc(-c3cc(-n4c5ccccc5c5ccccc54)cc(-n4c5ccccc5c5ccccc54)c3)c2)cc1 ; O=P(c1ccccc1)(c1ccccc1)c1cccc(-n2nc(-c3ccccc3)c3c(-c4ccccc4)cc4ccccc4c32)c1]

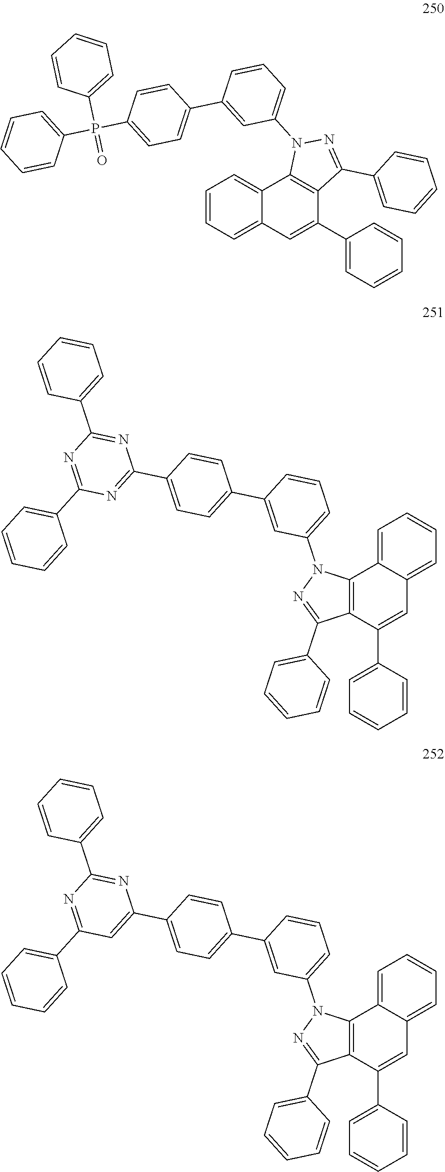 [SMILES: O=P(c1ccccc1)(c1ccccc1)c1ccc(-c2cccc(-n3nc(-c4ccccc4)c4c(-c5ccccc5)cc5ccccc5c43)c2)cc1 ; c1ccc(-c2nc(-c3ccccc3)nc(-c3ccc(-c4cccc(-n5nc(-c6ccccc6)c6c(-c7ccccc7)cc7ccccc7c65)c4)cc3)n2)cc1 ; c1ccc(-c2cc(-c3ccc(-c4cccc(-n5nc(-c6ccccc6)c6c(-c7ccccc7)cc7ccccc7c65)c4)cc3)nc(-c3ccccc3)n2)cc1]

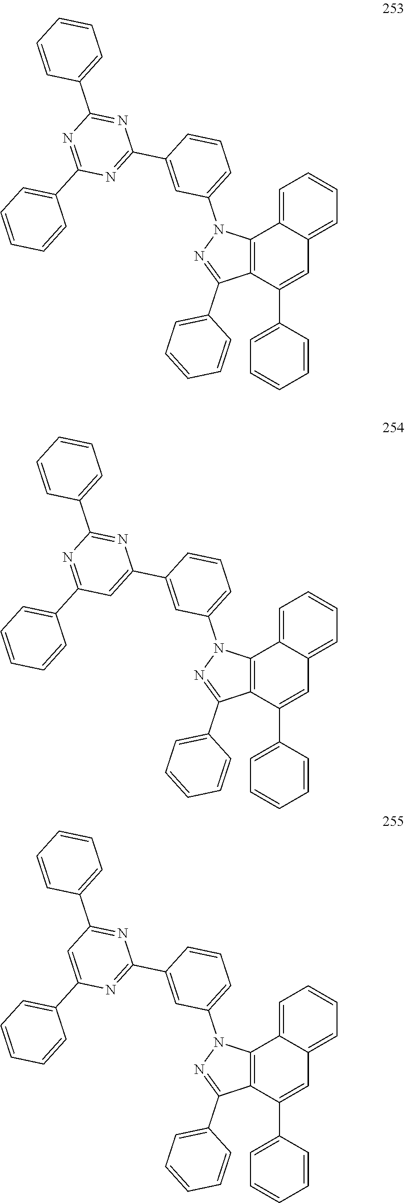 [SMILES: c1ccc(-c2nc(-c3ccccc3)nc(-c3cccc(-n4nc(-c5ccccc5)c5c(-c6ccccc6)cc6ccccc6c54)c3)n2)cc1 ; c1ccc(-c2cc(-c3cccc(-n4nc(-c5ccccc5)c5c(-c6ccccc6)cc6ccccc6c54)c3)nc(-c3ccccc3)n2)cc1 ; c1ccc(-c2cc(-c3ccccc3)nc(-c3cccc(-n4nc(-c5ccccc5)c5c(-c6ccccc6)cc6ccccc6c54)c3)n2)cc1]

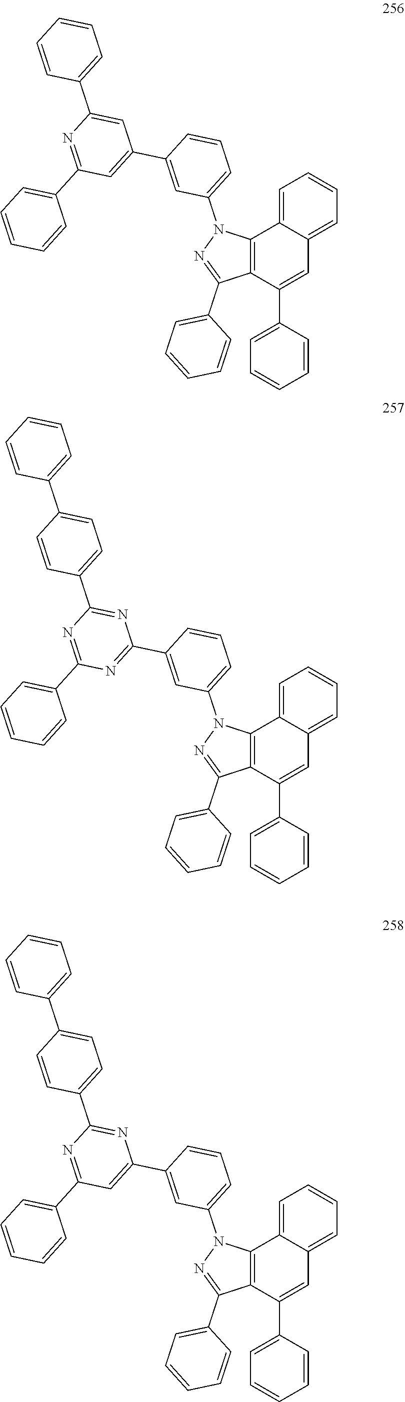 [SMILES: c1ccc(-c2cc(-c3cccc(-n4nc(-c5ccccc5)c5c(-c6ccccc6)cc6ccccc6c54)c3)cc(-c3ccccc3)n2)cc1 ; c1ccc(-c2ccc(-c3nc(-c4ccccc4)nc(-c4cccc(-n5nc(-c6ccccc6)c6c(-c7ccccc7)cc7ccccc7c65)c4)n3)cc2)cc1 ; c1ccc(-c2ccc(-c3nc(-c4ccccc4)cc(-c4cccc(-n5nc(-c6ccccc6)c6c(-c7ccccc7)cc7ccccc7c65)c4)n3)cc2)cc1]

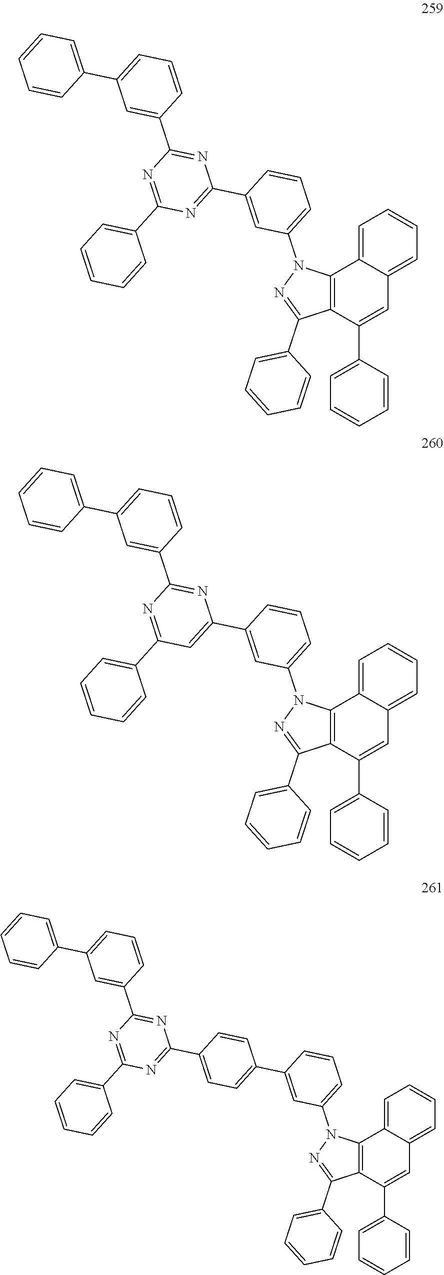 [SMILES: c1ccc(-c2cccc(-c3nc(-c4ccccc4)nc(-c4cccc(-n5nc(-c6ccccc6)c6c(-c7ccccc7)cc7ccccc7c65)c4)n3)c2)cc1 ; c1ccc(-c2cccc(-c3nc(-c4ccccc4)cc(-c4cccc(-n5nc(-c6ccccc6)c6c(-c7ccccc7)cc7ccccc7c65)c4)n3)c2)cc1 ; c1ccc(-c2cccc(-c3nc(-c4ccccc4)nc(-c4ccc(-c5cccc(-n6nc(-c7ccccc7)c7c(-c8ccccc8)cc8ccccc8c76)c5)cc4)n3)c2)cc1]

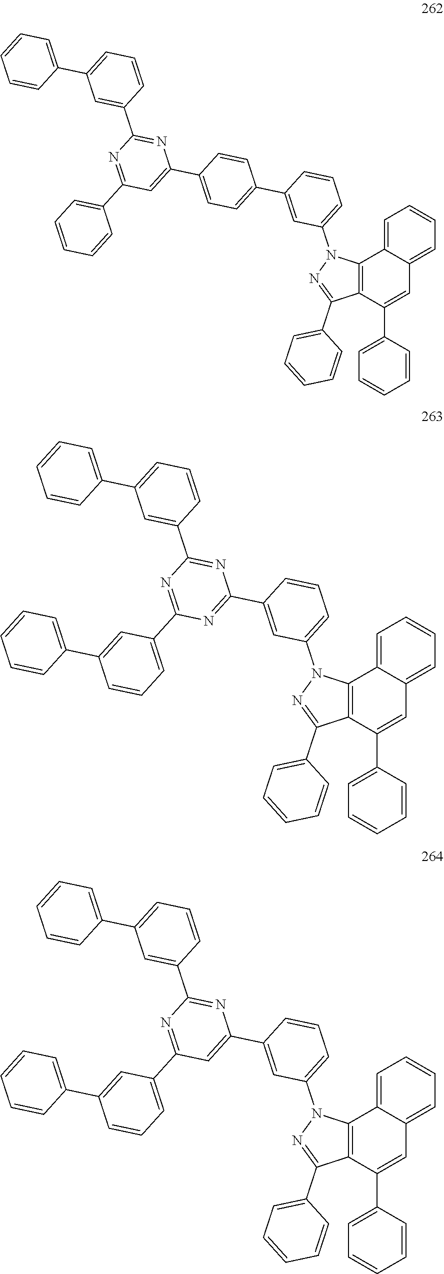 [SMILES: c1ccc(-c2cccc(-c3nc(-c4ccccc4)cc(-c4ccc(-c5cccc(-n6nc(-c7ccccc7)c7c(-c8ccccc8)cc8ccccc8c76)c5)cc4)n3)c2)cc1 ; c1ccc(-c2cccc(-c3nc(-c4cccc(-c5ccccc5)c4)nc(-c4cccc(-n5nc(-c6ccccc6)c6c(-c7ccccc7)cc7ccccc7c65)c4)n3)c2)cc1 ; c1ccc(-c2cccc(-c3cc(-c4cccc(-n5nc(-c6ccccc6)c6c(-c7ccccc7)cc7ccccc7c65)c4)nc(-c4cccc(-c5ccccc5)c4)n3)c2)cc1]

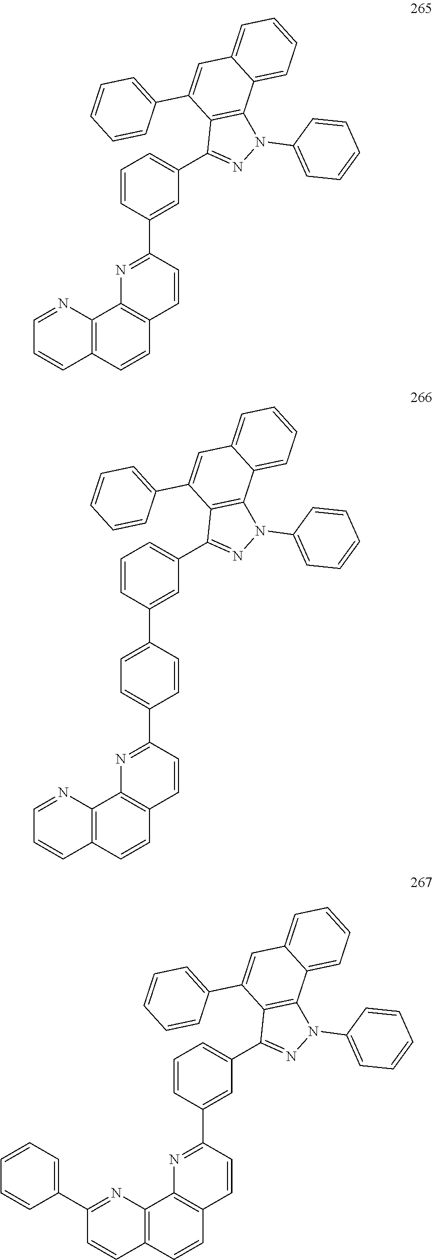 [SMILES: c1ccc(-c2cc3ccccc3c3c2c(-c2cccc(-c4ccc5ccc6cccnc6c5n4)c2)nn3-c2ccccc2)cc1 ; c1ccc(-c2cc3ccccc3c3c2c(-c2cccc(-c4ccc(-c5ccc6ccc7cccnc7c6n5)cc4)c2)nn3-c2ccccc2)cc1 ; c1ccc(-c2ccc3ccc4ccc(-c5cccc(-c6nn(-c7ccccc7)c7c6c(-c6ccccc6)cc6ccccc67)c5)nc4c3n2)cc1]

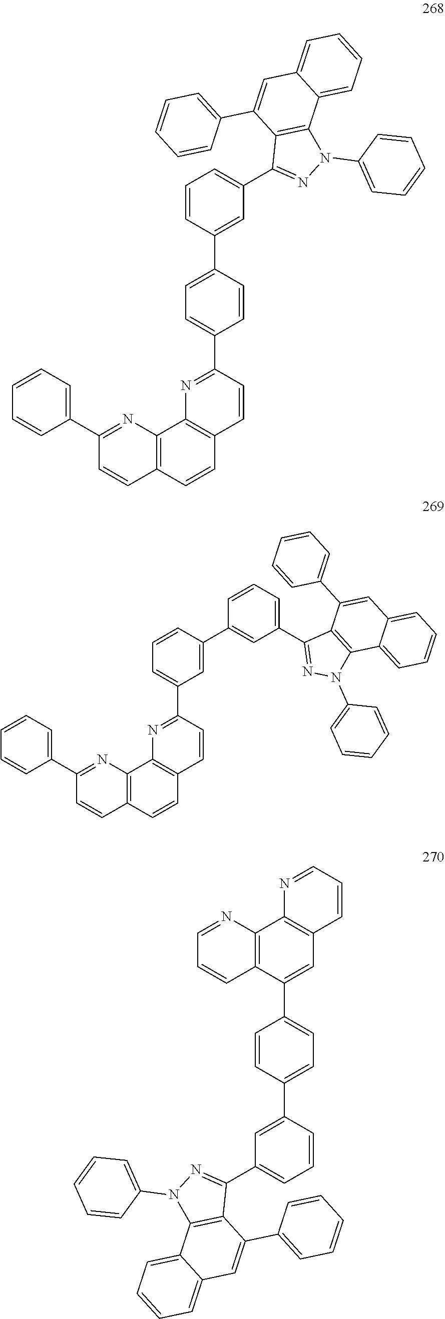 [SMILES: c1ccc(-c2ccc3ccc4ccc(-c5ccc(-c6cccc(-c7nn(-c8ccccc8)c8c7c(-c7ccccc7)cc7ccccc78)c6)cc5)nc4c3n2)cc1 ; c1ccc(-c2ccc3ccc4ccc(-c5cccc(-c6cccc(-c7nn(-c8ccccc8)c8c7c(-c7ccccc7)cc7ccccc78)c6)c5)nc4c3n2)cc1 ; c1ccc(-c2cc3ccccc3c3c2c(-c2cccc(-c4ccc(-c5cc6cccnc6c6ncccc56)cc4)c2)nn3-c2ccccc2)cc1]

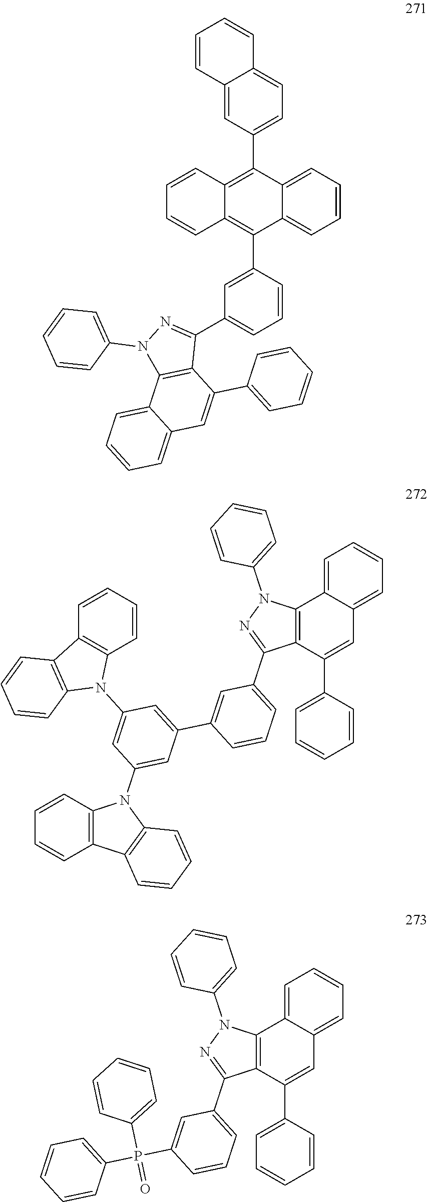 [SMILES: c1ccc(-c2cc3ccccc3c3c2c(-c2cccc(-c4c5ccccc5c(-c5ccc6ccccc6c5)c5ccccc45)c2)nn3-c2ccccc2)cc1 ; c1ccc(-c2cc3ccccc3c3c2c(-c2cccc(-c4cc(-n5c6ccccc6c6ccccc65)cc(-n5c6ccccc6c6ccccc65)c4)c2)nn3-c2ccccc2)cc1 ; O=P(c1ccccc1)(c1ccccc1)c1cccc(-c2nn(-c3ccccc3)c3c2c(-c2ccccc2)cc2ccccc23)c1]

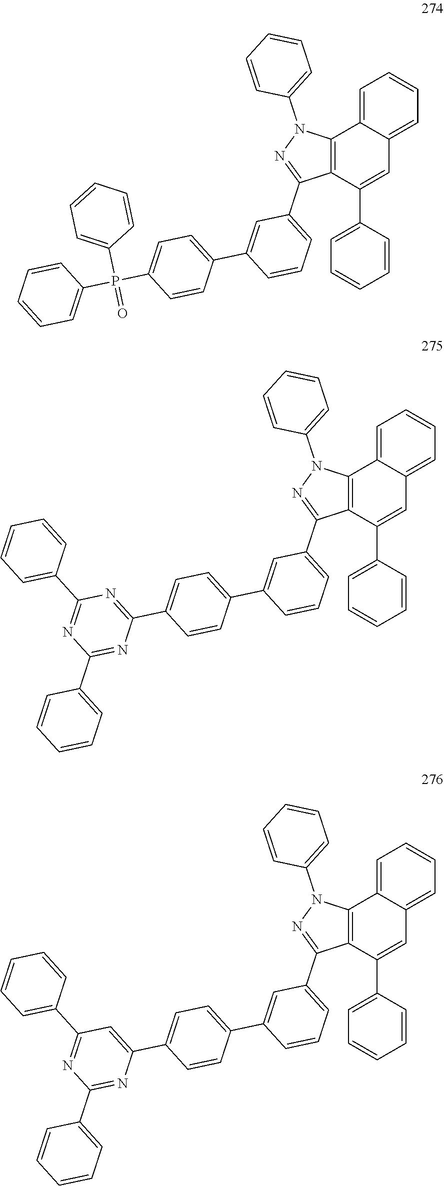 [SMILES: O=P(c1ccccc1)(c1ccccc1)c1ccc(-c2cccc(-c3nn(-c4ccccc4)c4c3c(-c3ccccc3)cc3ccccc34)c2)cc1 ; c1ccc(-c2nc(-c3ccccc3)nc(-c3ccc(-c4cccc(-c5nn(-c6ccccc6)c6c5c(-c5ccccc5)cc5ccccc56)c4)cc3)n2)cc1 ; c1ccc(-c2cc(-c3ccc(-c4cccc(-c5nn(-c6ccccc6)c6c5c(-c5ccccc5)cc5ccccc56)c4)cc3)nc(-c3ccccc3)n2)cc1]

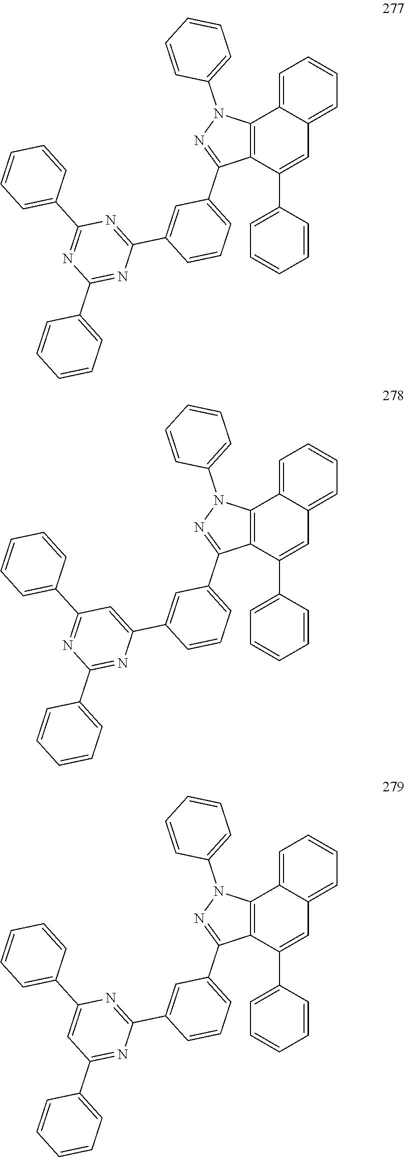 [SMILES: c1ccc(-c2nc(-c3ccccc3)nc(-c3cccc(-c4nn(-c5ccccc5)c5c4c(-c4ccccc4)cc4ccccc45)c3)n2)cc1 ; c1ccc(-c2cc(-c3cccc(-c4nn(-c5ccccc5)c5c4c(-c4ccccc4)cc4ccccc45)c3)nc(-c3ccccc3)n2)cc1 ; c1ccc(-c2cc(-c3ccccc3)nc(-c3cccc(-c4nn(-c5ccccc5)c5c4c(-c4ccccc4)cc4ccccc45)c3)n2)cc1]

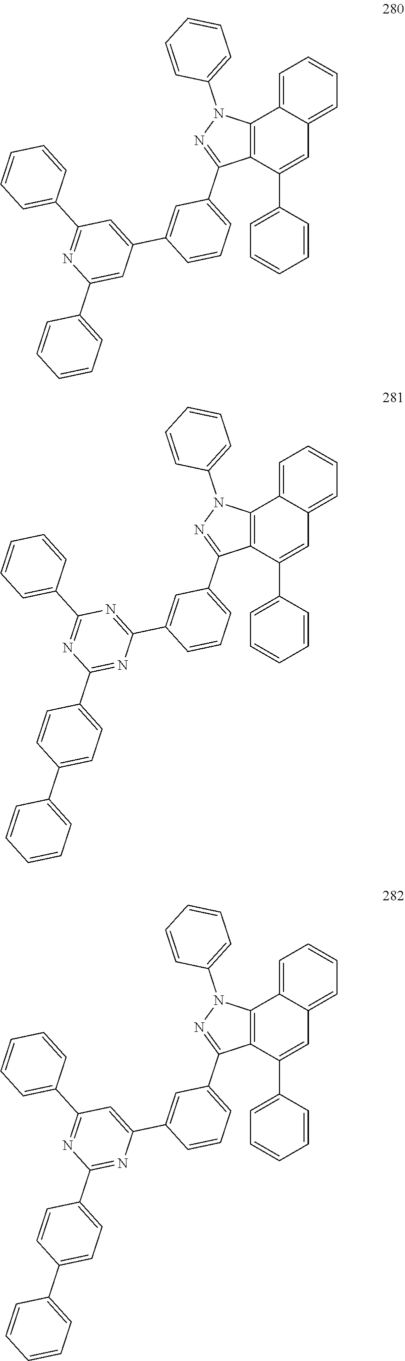 [SMILES: c1ccc(-c2cc(-c3cccc(-c4nn(-c5ccccc5)c5c4c(-c4ccccc4)cc4ccccc45)c3)cc(-c3ccccc3)n2)cc1 ; c1ccc(-c2ccc(-c3nc(-c4ccccc4)nc(-c4cccc(-c5nn(-c6ccccc6)c6c5c(-c5ccccc5)cc5ccccc56)c4)n3)cc2)cc1 ; c1ccc(-c2ccc(-c3nc(-c4ccccc4)cc(-c4cccc(-c5nn(-c6ccccc6)c6c5c(-c5ccccc5)cc5ccccc56)c4)n3)cc2)cc1]

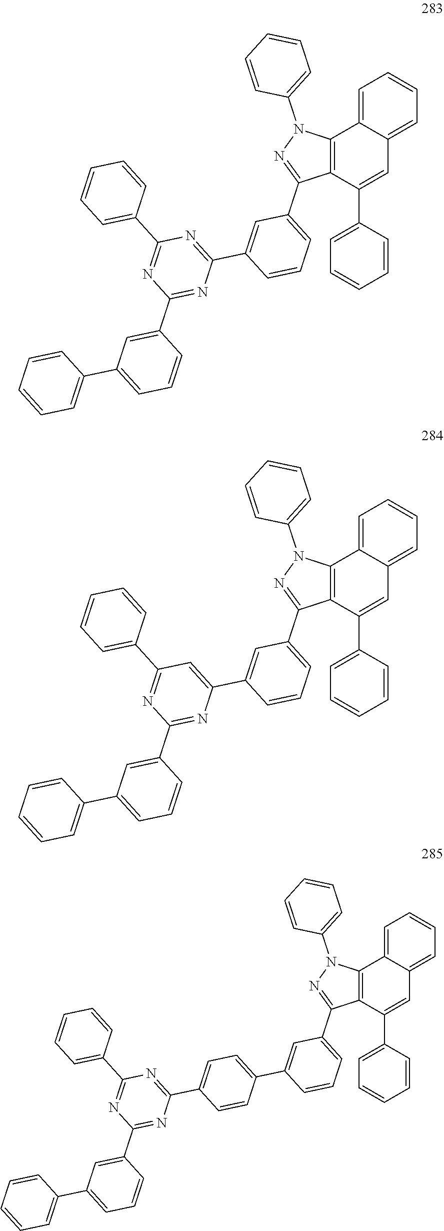 [SMILES: c1ccc(-c2cccc(-c3nc(-c4ccccc4)nc(-c4cccc(-c5nn(-c6ccccc6)c6c5c(-c5ccccc5)cc5ccccc56)c4)n3)c2)cc1 ; c1ccc(-c2cccc(-c3nc(-c4ccccc4)cc(-c4cccc(-c5nn(-c6ccccc6)c6c5c(-c5ccccc5)cc5ccccc56)c4)n3)c2)cc1 ; c1ccc(-c2cccc(-c3nc(-c4ccccc4)nc(-c4ccc(-c5cccc(-c6nn(-c7ccccc7)c7c6c(-c6ccccc6)cc6ccccc67)c5)cc4)n3)c2)cc1]

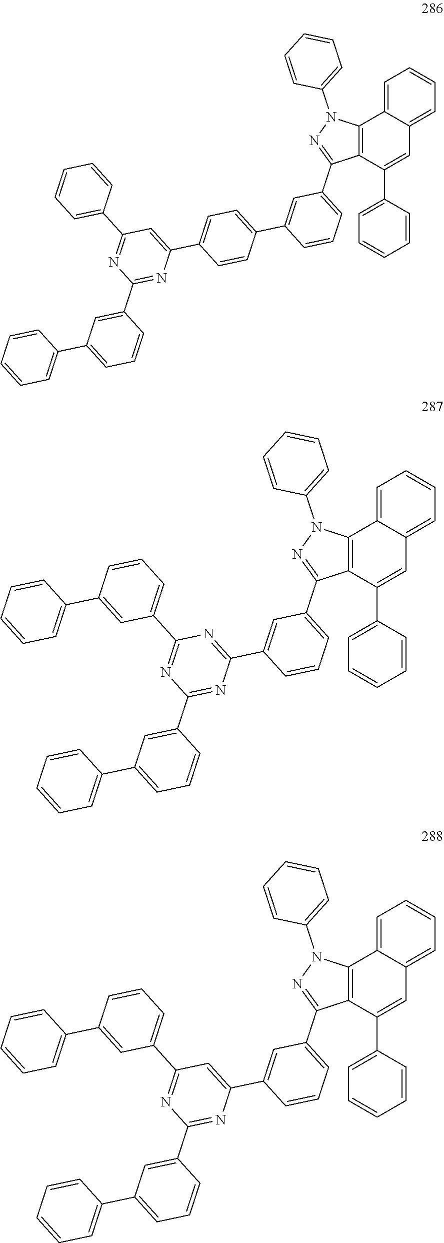 [SMILES: c1ccc(-c2cccc(-c3nc(-c4ccccc4)cc(-c4ccc(-c5cccc(-c6nn(-c7ccccc7)c7c6c(-c6ccccc6)cc6ccccc67)c5)cc4)n3)c2)cc1 ; c1ccc(-c2cccc(-c3nc(-c4cccc(-c5ccccc5)c4)nc(-c4cccc(-c5nn(-c6ccccc6)c6c5c(-c5ccccc5)cc5ccccc56)c4)n3)c2)cc1 ; c1ccc(-c2cccc(-c3cc(-c4cccc(-c5nn(-c6ccccc6)c6c5c(-c5ccccc5)cc5ccccc56)c4)nc(-c4cccc(-c5ccccc5)c4)n3)c2)cc1]

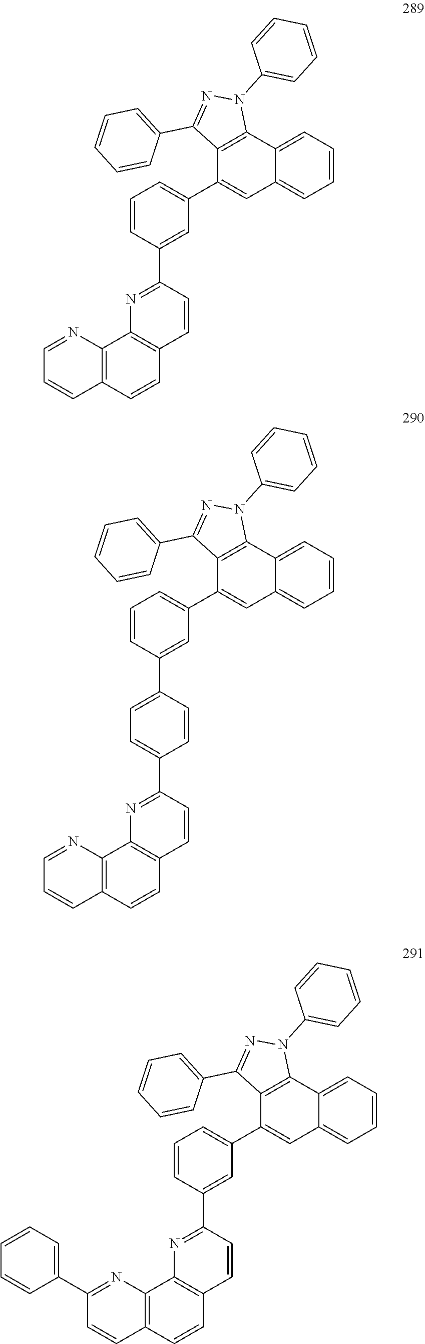 [SMILES: c1ccc(-c2nn(-c3ccccc3)c3c2c(-c2cccc(-c4ccc5ccc6cccnc6c5n4)c2)cc2ccccc23)cc1 ; c1ccc(-c2nn(-c3ccccc3)c3c2c(-c2cccc(-c4ccc(-c5ccc6ccc7cccnc7c6n5)cc4)c2)cc2ccccc23)cc1 ; c1ccc(-c2ccc3ccc4ccc(-c5cccc(-c6cc7ccccc7c7c6c(-c6ccccc6)nn7-c6ccccc6)c5)nc4c3n2)cc1]

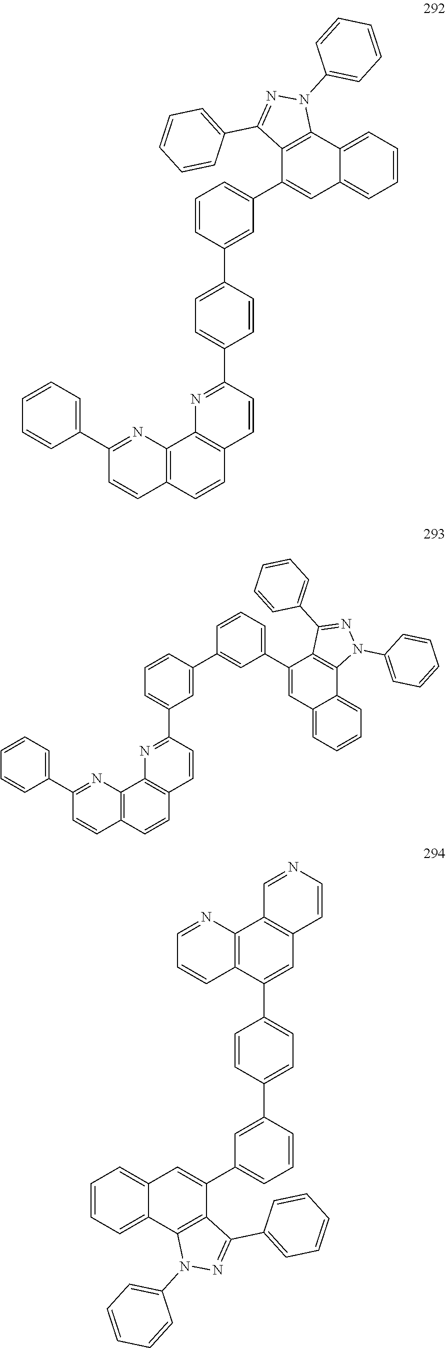 [SMILES: c1ccc(-c2ccc3ccc4ccc(-c5ccc(-c6cccc(-c7cc8ccccc8c8c7c(-c7ccccc7)nn8-c7ccccc7)c6)cc5)nc4c3n2)cc1 ; c1ccc(-c2ccc3ccc4ccc(-c5cccc(-c6cccc(-c7cc8ccccc8c8c7c(-c7ccccc7)nn8-c7ccccc7)c6)c5)nc4c3n2)cc1 ; c1ccc(-c2nn(-c3ccccc3)c3c2c(-c2cccc(-c4ccc(-c5cc6ccncc6c6ncccc56)cc4)c2)cc2ccccc23)cc1]

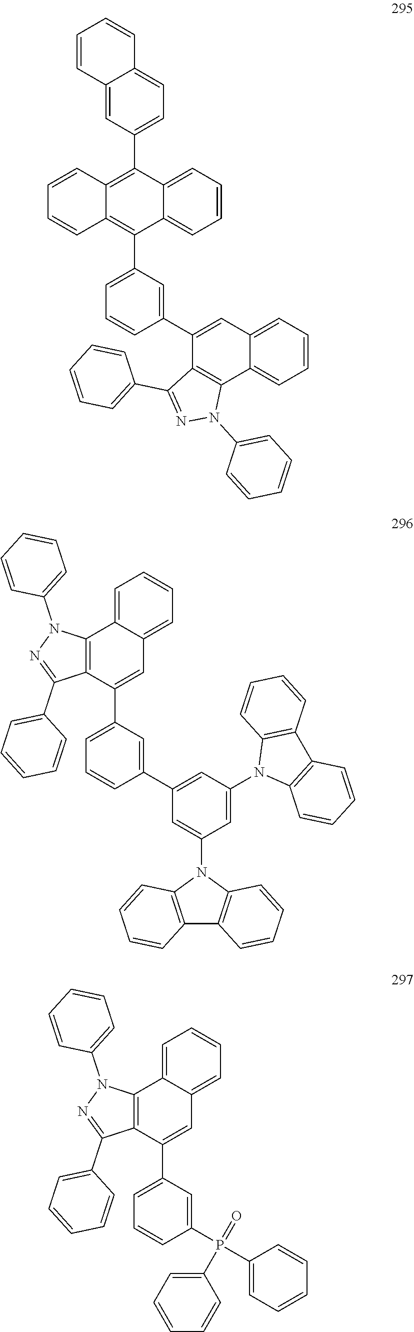 [SMILES: c1ccc(-c2nn(-c3ccccc3)c3c2c(-c2cccc(-c4c5ccccc5c(-c5ccc6ccccc6c5)c5ccccc45)c2)cc2ccccc23)cc1 ; c1ccc(-c2nn(-c3ccccc3)c3c2c(-c2cccc(-c4cc(-n5c6ccccc6c6ccccc65)cc(-n5c6ccccc6c6ccccc65)c4)c2)cc2ccccc23)cc1 ; O=P(c1ccccc1)(c1ccccc1)c1cccc(-c2cc3ccccc3c3c2c(-c2ccccc2)nn3-c2ccccc2)c1]

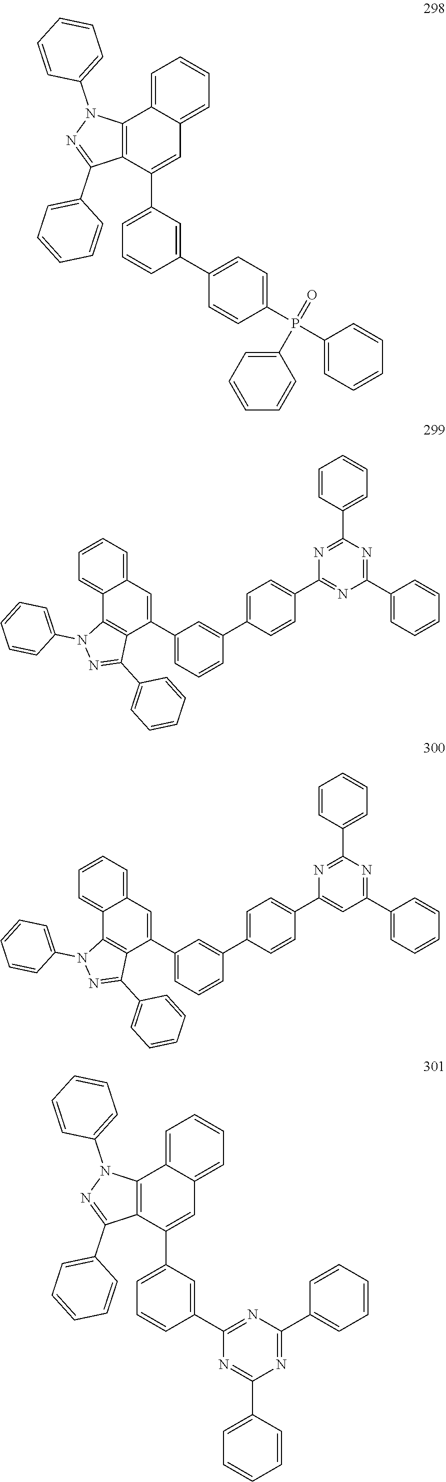 [SMILES: O=P(c1ccccc1)(c1ccccc1)c1ccc(-c2cccc(-c3cc4ccccc4c4c3c(-c3ccccc3)nn4-c3ccccc3)c2)cc1 ; c1ccc(-c2nc(-c3ccccc3)nc(-c3ccc(-c4cccc(-c5cc6ccccc6c6c5c(-c5ccccc5)nn6-c5ccccc5)c4)cc3)n2)cc1 ; c1ccc(-c2cc(-c3ccc(-c4cccc(-c5cc6ccccc6c6c5c(-c5ccccc5)nn6-c5ccccc5)c4)cc3)nc(-c3ccccc3)n2)cc1 ; c1ccc(-c2nc(-c3ccccc3)nc(-c3cccc(-c4cc5ccccc5c5c4c(-c4ccccc4)nn5-c4ccccc4)c3)n2)cc1]

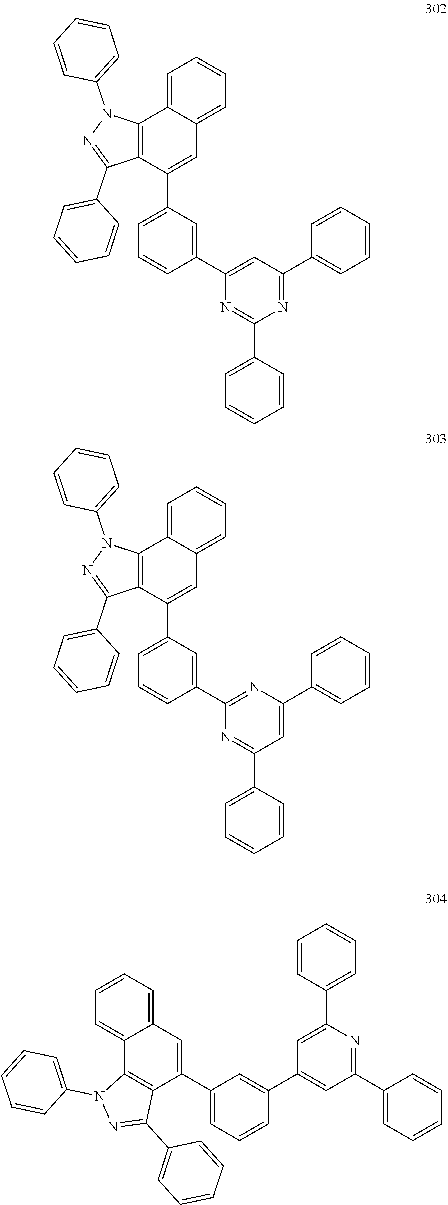 [SMILES: c1ccc(-c2cc(-c3cccc(-c4cc5ccccc5c5c4c(-c4ccccc4)nn5-c4ccccc4)c3)nc(-c3ccccc3)n2)cc1 ; c1ccc(-c2cc(-c3ccccc3)nc(-c3cccc(-c4cc5ccccc5c5c4c(-c4ccccc4)nn5-c4ccccc4)c3)n2)cc1 ; c1ccc(-c2cc(-c3cccc(-c4cc5ccccc5c5c4c(-c4ccccc4)nn5-c4ccccc4)c3)cc(-c3ccccc3)n2)cc1]

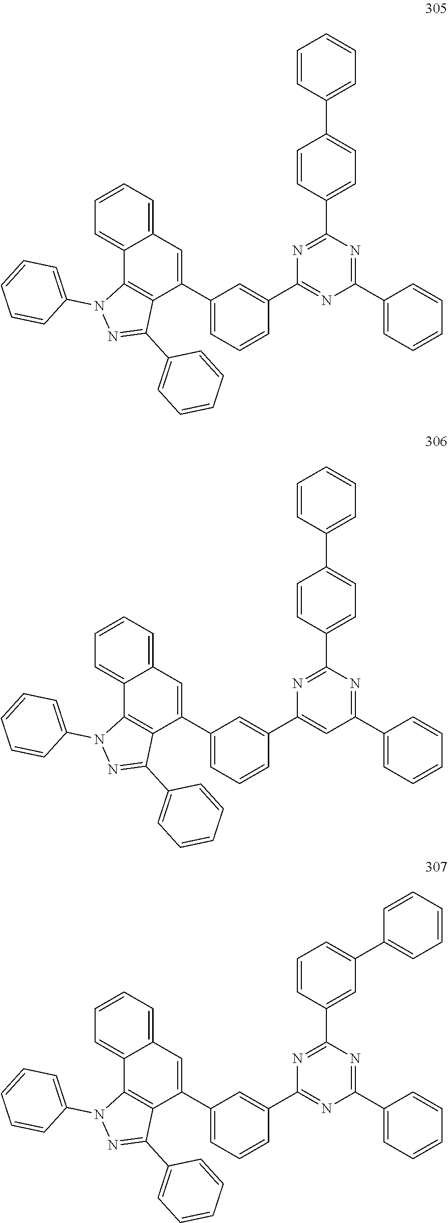 [SMILES: c1ccc(-c2ccc(-c3nc(-c4ccccc4)nc(-c4cccc(-c5cc6ccccc6c6c5c(-c5ccccc5)nn6-c5ccccc5)c4)n3)cc2)cc1 ; c1ccc(-c2ccc(-c3nc(-c4ccccc4)cc(-c4cccc(-c5cc6ccccc6c6c5c(-c5ccccc5)nn6-c5ccccc5)c4)n3)cc2)cc1 ; c1ccc(-c2cccc(-c3nc(-c4ccccc4)nc(-c4cccc(-c5cc6ccccc6c6c5c(-c5ccccc5)nn6-c5ccccc5)c4)n3)c2)cc1]

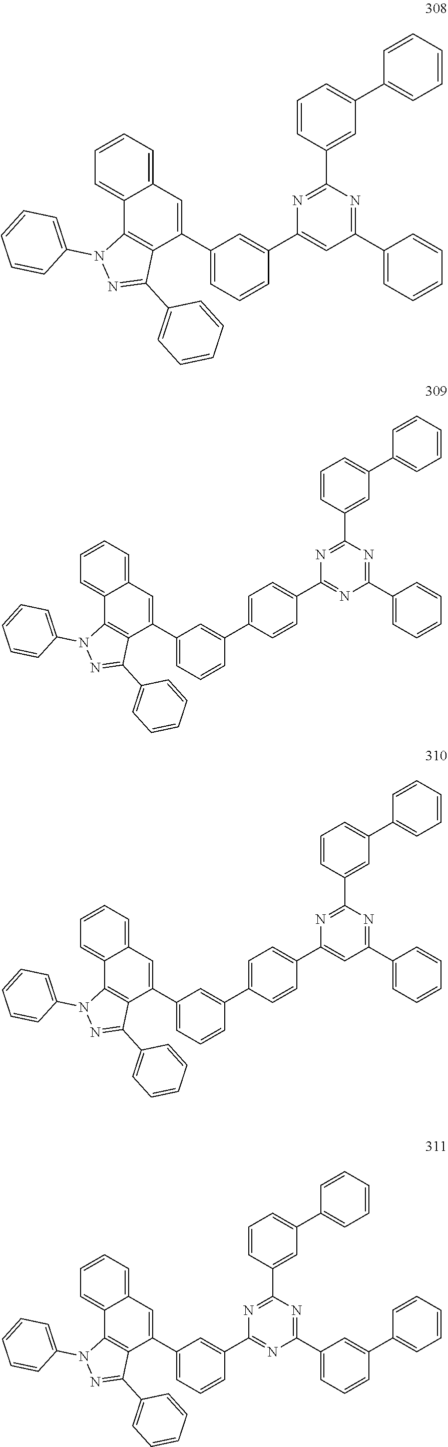 [SMILES: c1ccc(-c2cccc(-c3nc(-c4ccccc4)cc(-c4cccc(-c5cc6ccccc6c6c5c(-c5ccccc5)nn6-c5ccccc5)c4)n3)c2)cc1 ; c1ccc(-c2cccc(-c3nc(-c4ccccc4)nc(-c4ccc(-c5cccc(-c6cc7ccccc7c7c6c(-c6ccccc6)nn7-c6ccccc6)c5)cc4)n3)c2)cc1 ; c1ccc(-c2cccc(-c3nc(-c4ccccc4)cc(-c4ccc(-c5cccc(-c6cc7ccccc7c7c6c(-c6ccccc6)nn7-c6ccccc6)c5)cc4)n3)c2)cc1 ; c1ccc(-c2cccc(-c3nc(-c4cccc(-c5ccccc5)c4)nc(-c4cccc(-c5cc6ccccc6c6c5c(-c5ccccc5)nn6-c5ccccc5)c4)n3)c2)cc1]

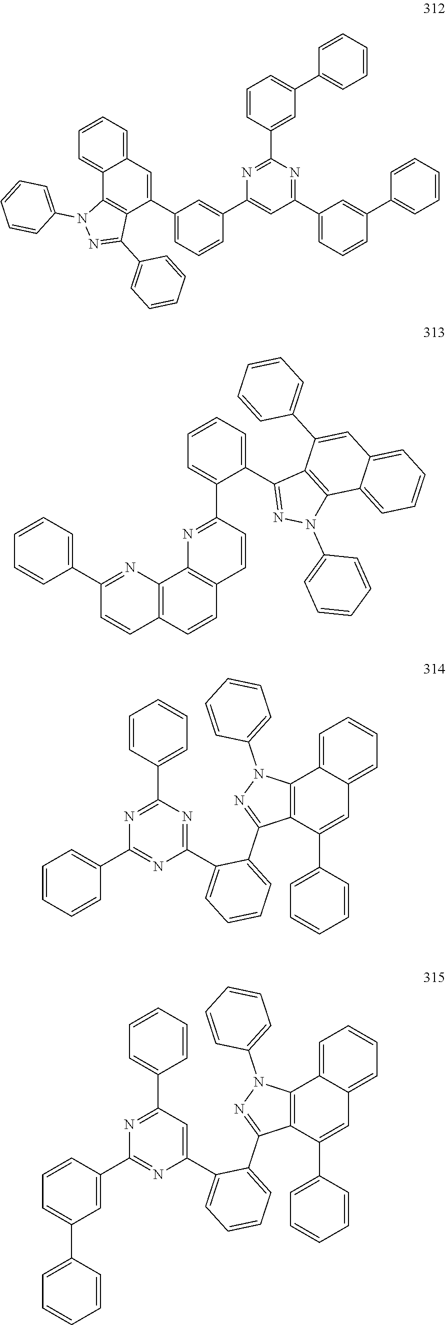 [SMILES: c1ccc(-c2cccc(-c3cc(-c4cccc(-c5cc6ccccc6c6c5c(-c5ccccc5)nn6-c5ccccc5)c4)nc(-c4cccc(-c5ccccc5)c4)n3)c2)cc1 ; c1ccc(-c2ccc3ccc4ccc(-c5ccccc5-c5nn(-c6ccccc6)c6c5c(-c5ccccc5)cc5ccccc56)nc4c3n2)cc1 ; c1ccc(-c2nc(-c3ccccc3)nc(-c3ccccc3-c3nn(-c4ccccc4)c4c3c(-c3ccccc3)cc3ccccc34)n2)cc1 ; c1ccc(-c2cccc(-c3nc(-c4ccccc4)cc(-c4ccccc4-c4nn(-c5ccccc5)c5c4c(-c4ccccc4)cc4ccccc45)n3)c2)cc1]

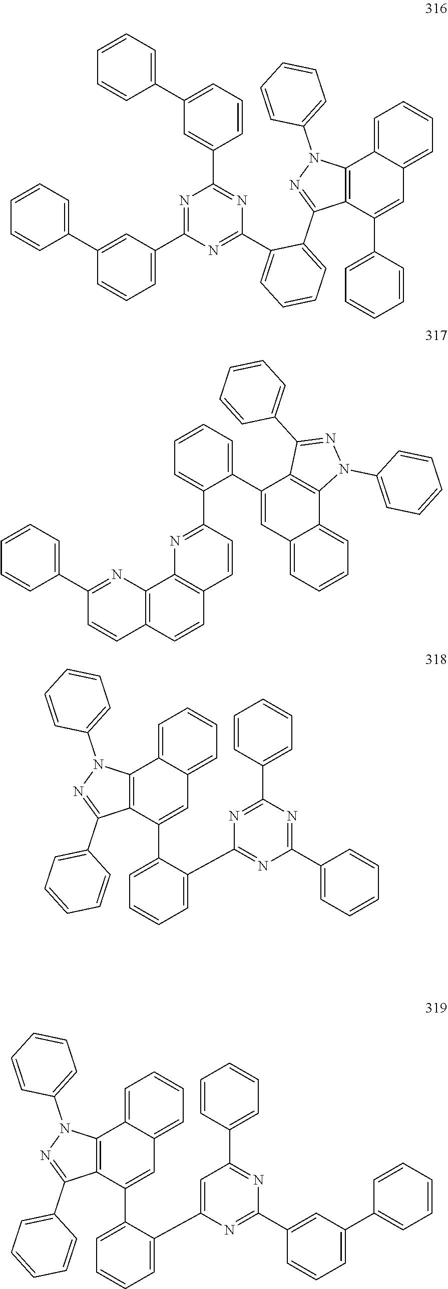

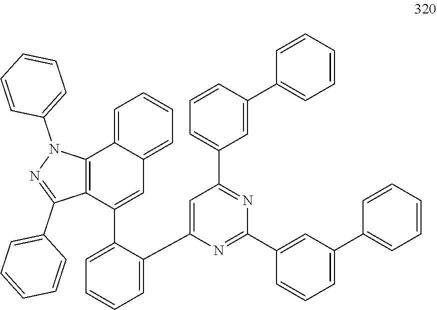 [SMILES: c1ccc(-c2cccc(-c3cc(-c4ccccc4-c4cc5ccccc5c5c4c(-c4ccccc4)nn5-c4ccccc4)nc(-c4cccc(-c5ccccc5)c4)n3)c2)cc1]

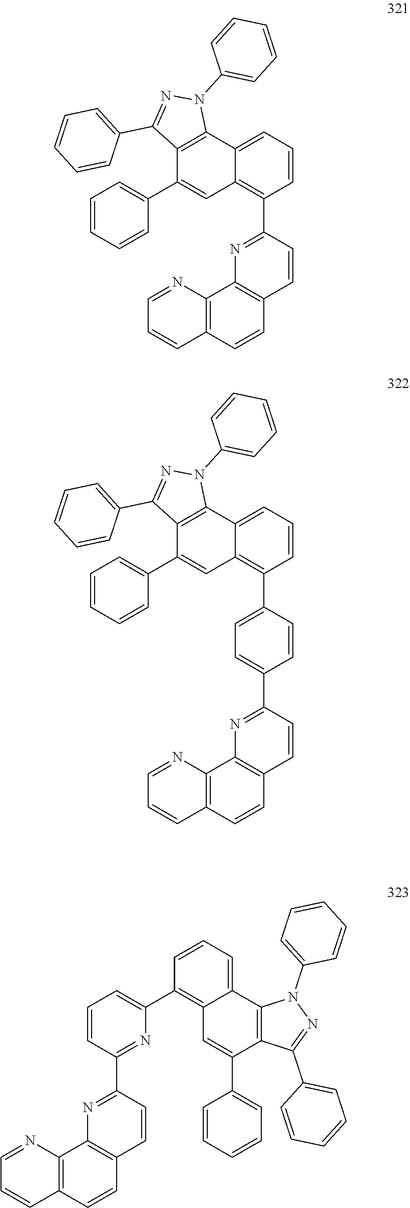

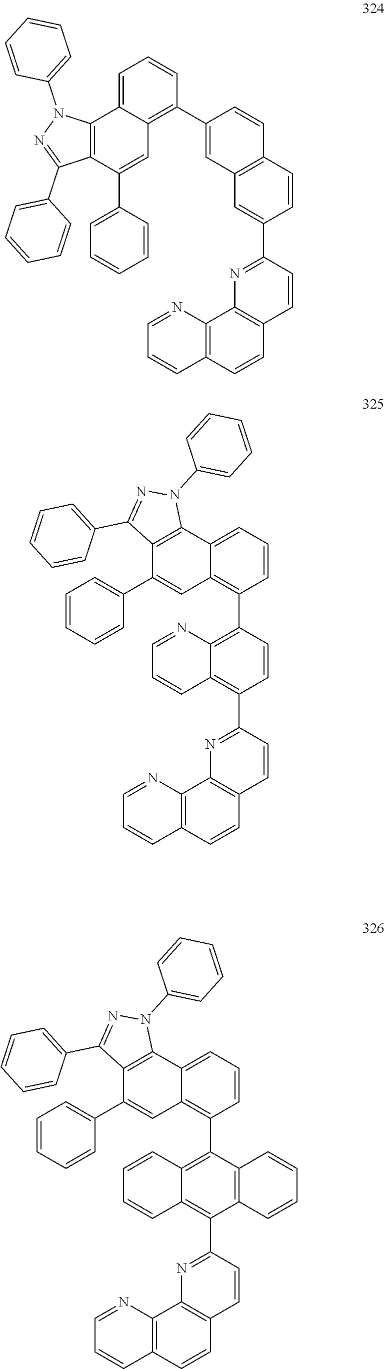 [SMILES: c1ccc(-c2cc3c(-c4ccc5ccc(-c6ccc7ccc8cccnc8c7n6)cc5c4)cccc3c3c2c(-c2ccccc2)nn3-c2ccccc2)cc1 ; c1ccc(-c2cc3c(-c4ccc(-c5ccc6ccc7cccnc7c6n5)c5cccnc45)cccc3c3c2c(-c2ccccc2)nn3-c2ccccc2)cc1 ; c1ccc(-c2cc3c(-c4c5ccccc5c(-c5ccc6ccc7cccnc7c6n5)c5ccccc45)cccc3c3c2c(-c2ccccc2)nn3-c2ccccc2)cc1]

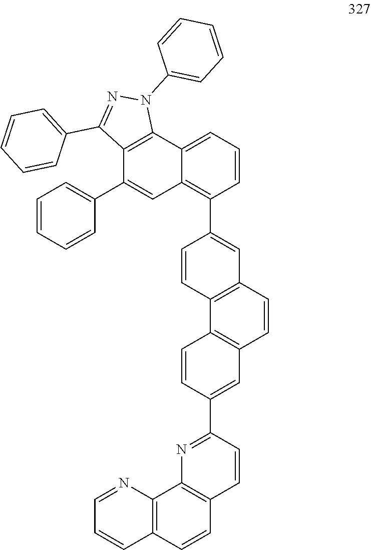 [SMILES: c1ccc(-c2cc3c(-c4ccc5c(ccc6cc(-c7ccc8ccc9cccnc9c8n7)ccc65)c4)cccc3c3c2c(-c2ccccc2)nn3-c2ccccc2)cc1]

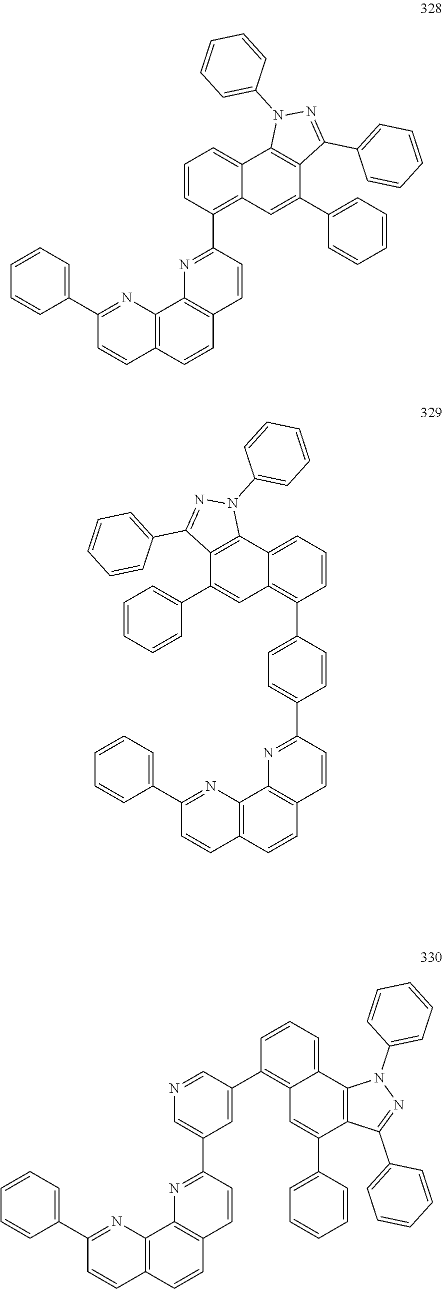 [SMILES: c1ccc(-c2ccc3ccc4ccc(-c5cccc6c5cc(-c5ccccc5)c5c(-c7ccccc7)nn(-c7ccccc7)c56)nc4c3n2)cc1 ; c1ccc(-c2ccc3ccc4ccc(-c5ccc(-c6cccc7c6cc(-c6ccccc6)c6c(-c8ccccc8)nn(-c8ccccc8)c67)cc5)nc4c3n2)cc1 ; c1ccc(-c2ccc3ccc4ccc(-c5cncc(-c6cccc7c6cc(-c6ccccc6)c6c(-c8ccccc8)nn(-c8ccccc8)c67)c5)nc4c3n2)cc1]

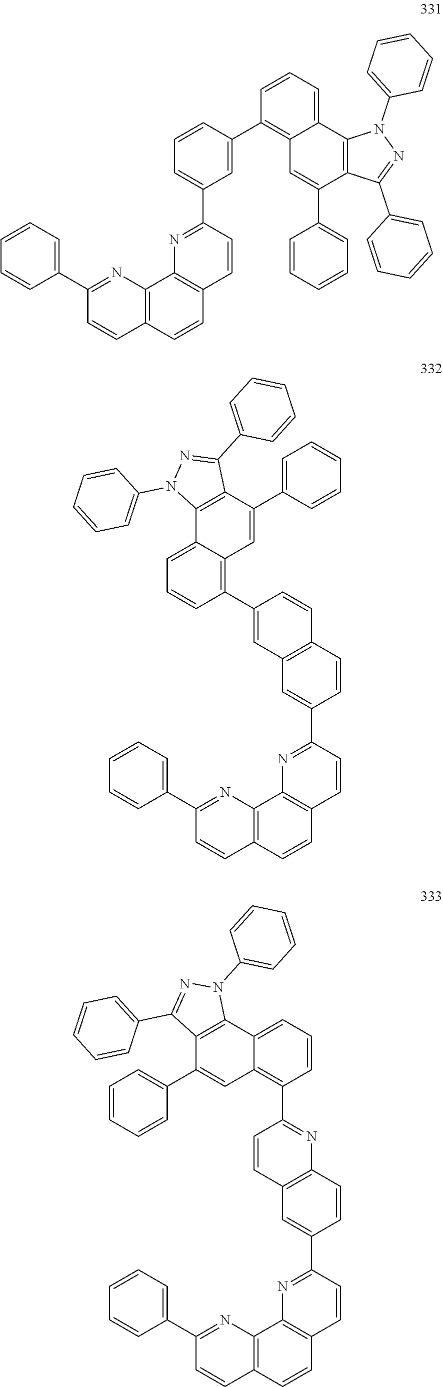 [SMILES: c1ccc(-c2ccc3ccc4ccc(-c5cccc(-c6cccc7c6cc(-c6ccccc6)c6c(-c8ccccc8)nn(-c8ccccc8)c67)c5)nc4c3n2)cc1 ; c1ccc(-c2ccc3ccc4ccc(-c5ccc6ccc(-c7cccc8c7cc(-c7ccccc7)c7c(-c9ccccc9)nn(-c9ccccc9)c78)cc6c5)nc4c3n2)cc1 ; c1ccc(-c2ccc3ccc4ccc(-c5ccc6nc(-c7cccc8c7cc(-c7ccccc7)c7c(-c9ccccc9)nn(-c9ccccc9)c78)ccc6c5)nc4c3n2)cc1]

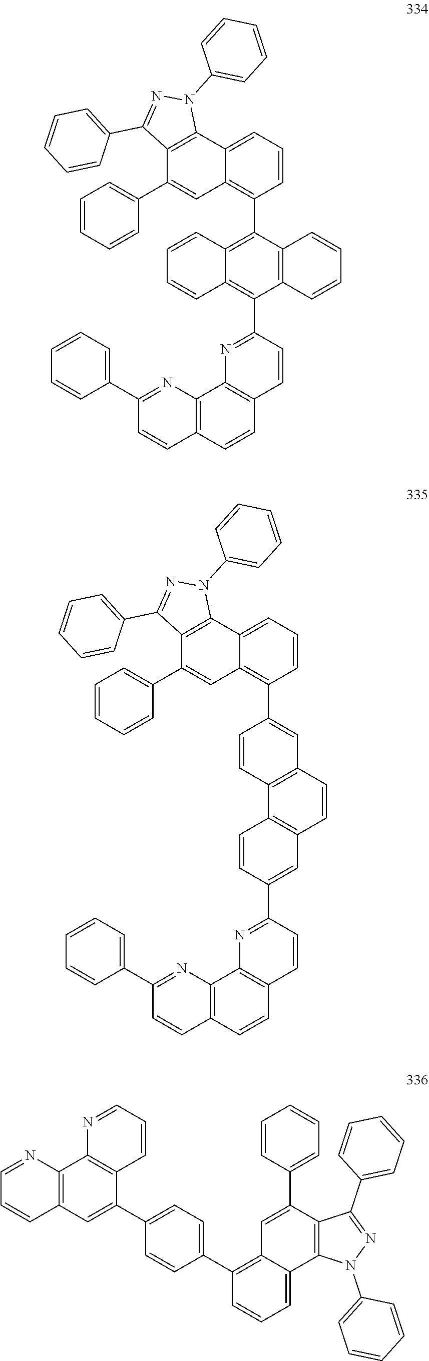 [SMILES: c1ccc(-c2ccc3ccc4ccc(-c5c6ccccc6c(-c6cccc7c6cc(-c6ccccc6)c6c(-c8ccccc8)nn(-c8ccccc8)c67)c6ccccc56)nc4c3n2)cc1 ; c1ccc(-c2ccc3ccc4ccc(-c5ccc6c(ccc7cc(-c8cccc9c8cc(-c8ccccc8)c8c(-c%10ccccc%10)nn(-c%10ccccc%10)c89)ccc76)c5)nc4c3n2)cc1 ; c1ccc(-c2cc3c(-c4ccc(-c5cc6cccnc6c6ncccc56)cc4)cccc3c3c2c(-c2ccccc2)nn3-c2ccccc2)cc1]

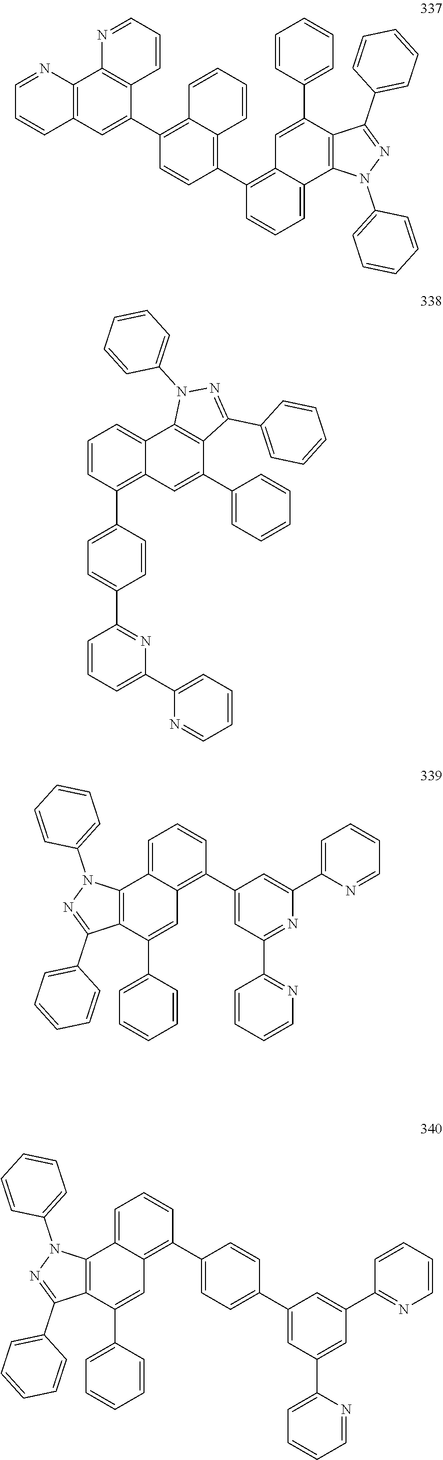 [SMILES: c1ccc(-c2cc3c(-c4ccc(-c5cc6cccnc6c6ncccc56)c5ccccc45)cccc3c3c2c(-c2ccccc2)nn3-c2ccccc2)cc1 ; c1ccc(-c2cc3c(-c4ccc(-c5cccc(-c6ccccn6)n5)cc4)cccc3c3c2c(-c2ccccc2)nn3-c2ccccc2)cc1 ; c1ccc(-c2cc3c(-c4cc(-c5ccccn5)nc(-c5ccccn5)c4)cccc3c3c2c(-c2ccccc2)nn3-c2ccccc2)cc1 ; c1ccc(-c2cc3c(-c4ccc(-c5cc(-c6ccccn6)cc(-c6ccccn6)c5)cc4)cccc3c3c2c(-c2ccccc2)nn3-c2ccccc2)cc1]

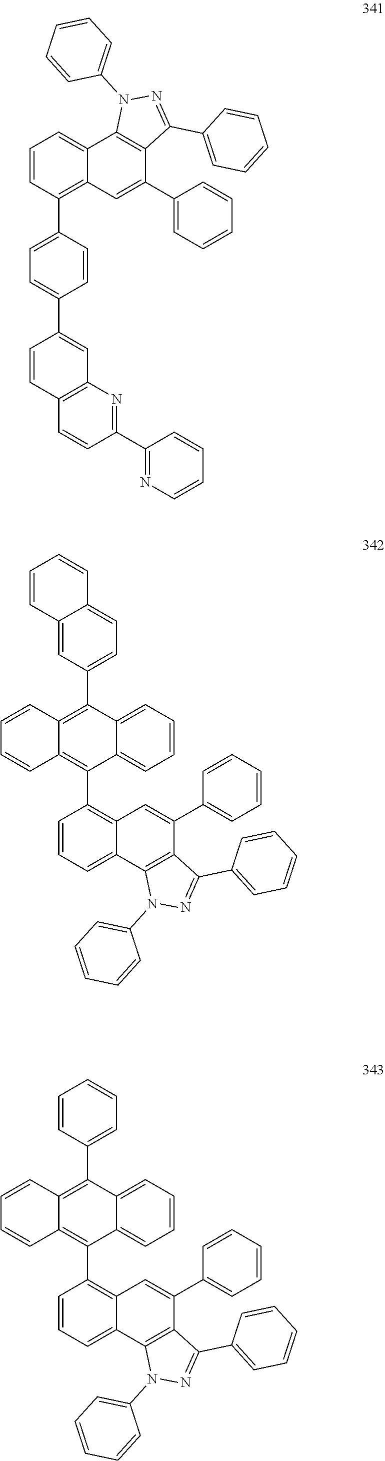 [SMILES: c1ccc(-c2cc3c(-c4ccc(-c5ccc6ccc(-c7ccccn7)nc6c5)cc4)cccc3c3c2c(-c2ccccc2)nn3-c2ccccc2)cc1 ; c1ccc(-c2cc3c(-c4c5ccccc5c(-c5ccc6ccccc6c5)c5ccccc45)cccc3c3c2c(-c2ccccc2)nn3-c2ccccc2)cc1 ; c1ccc(-c2c3ccccc3c(-c3cccc4c3cc(-c3ccccc3)c3c(-c5ccccc5)nn(-c5ccccc5)c34)c3ccccc23)cc1]

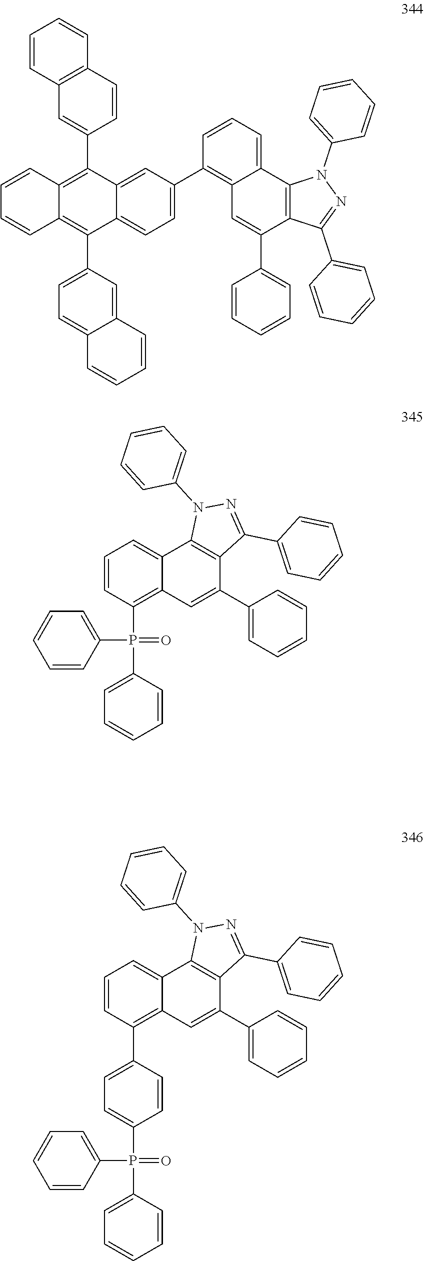 [SMILES: c1ccc(-c2cc3c(-c4ccc5c(-c6ccc7ccccc7c6)c6ccccc6c(-c6ccc7ccccc7c6)c5c4)cccc3c3c2c(-c2ccccc2)nn3-c2ccccc2)cc1 ; O=P(c1ccccc1)(c1ccccc1)c1cccc2c1cc(-c1ccccc1)c1c(-c3ccccc3)nn(-c3ccccc3)c12 ; O=P(c1ccccc1)(c1ccccc1)c1ccc(-c2cccc3c2cc(-c2ccccc2)c2c(-c4ccccc4)nn(-c4ccccc4)c23)cc1]

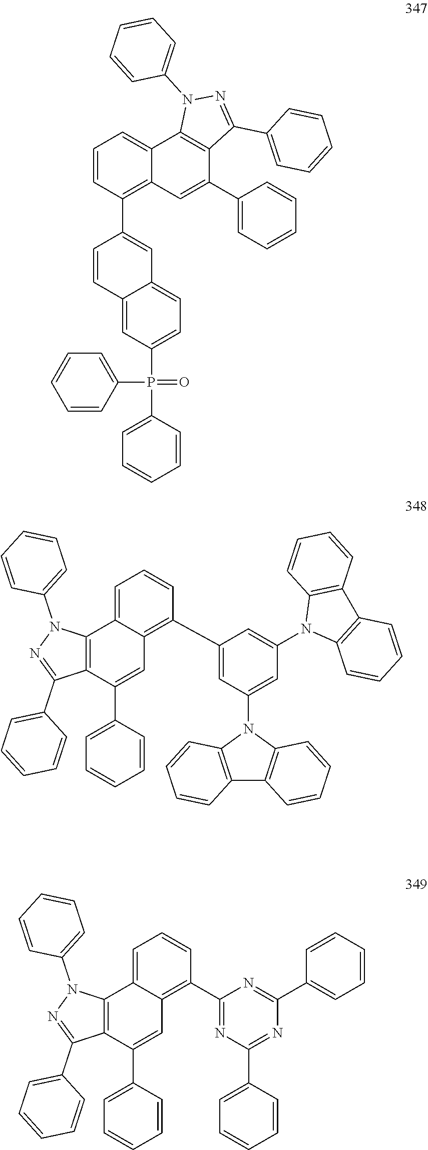 [SMILES: O=P(c1ccccc1)(c1ccccc1)c1ccc2cc(-c3cccc4c3cc(-c3ccccc3)c3c(-c5ccccc5)nn(-c5ccccc5)c34)ccc2c1 ; c1ccc(-c2cc3c(-c4cc(-n5c6ccccc6c6ccccc65)cc(-n5c6ccccc6c6ccccc65)c4)cccc3c3c2c(-c2ccccc2)nn3-c2ccccc2)cc1 ; c1ccc(-c2nc(-c3ccccc3)nc(-c3cccc4c3cc(-c3ccccc3)c3c(-c5ccccc5)nn(-c5ccccc5)c34)n2)cc1]

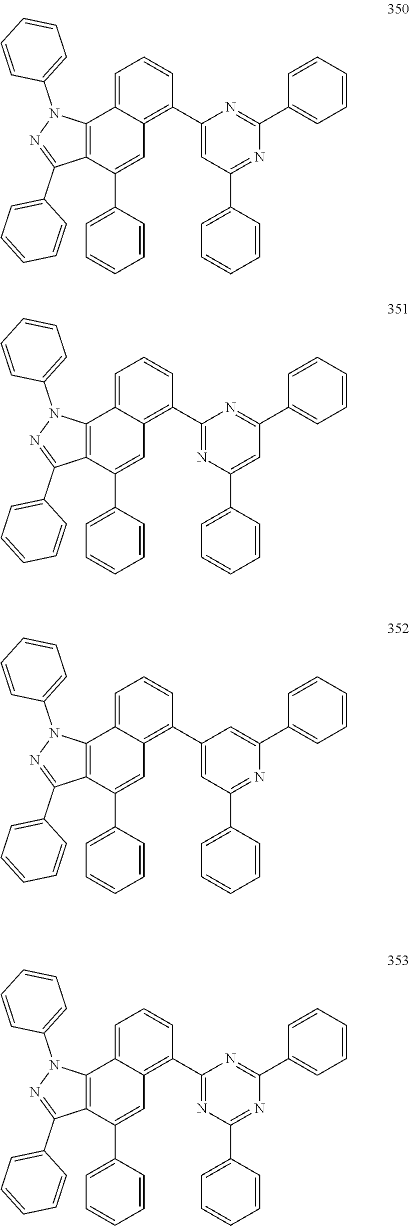 [SMILES: c1ccc(-c2cc(-c3cccc4c3cc(-c3ccccc3)c3c(-c5ccccc5)nn(-c5ccccc5)c34)nc(-c3ccccc3)n2)cc1 ; c1ccc(-c2cc(-c3ccccc3)nc(-c3cccc4c3cc(-c3ccccc3)c3c(-c5ccccc5)nn(-c5ccccc5)c34)n2)cc1 ; c1ccc(-c2cc(-c3cccc4c3cc(-c3ccccc3)c3c(-c5ccccc5)nn(-c5ccccc5)c34)cc(-c3ccccc3)n2)cc1 ; c1ccc(-c2nc(-c3ccccc3)nc(-c3cccc4c3cc(-c3ccccc3)c3c(-c5ccccc5)nn(-c5ccccc5)c34)n2)cc1]

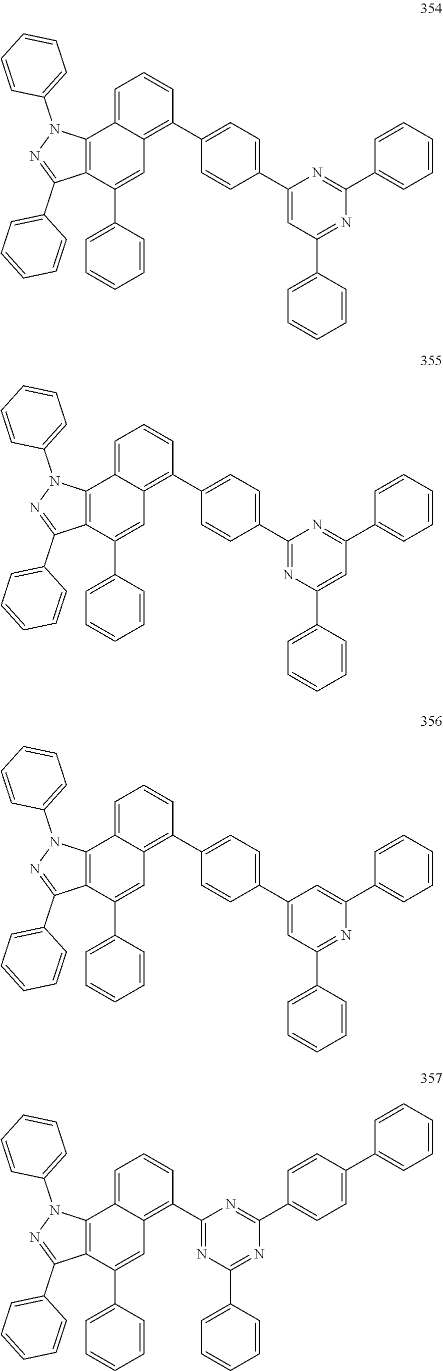 [SMILES: c1ccc(-c2cc(-c3ccc(-c4cccc5c4cc(-c4ccccc4)c4c(-c6ccccc6)nn(-c6ccccc6)c45)cc3)nc(-c3ccccc3)n2)cc1 ; c1ccc(-c2cc(-c3ccccc3)nc(-c3ccc(-c4cccc5c4cc(-c4ccccc4)c4c(-c6ccccc6)nn(-c6ccccc6)c45)cc3)n2)cc1 ; c1ccc(-c2cc(-c3ccc(-c4cccc5c4cc(-c4ccccc4)c4c(-c6ccccc6)nn(-c6ccccc6)c45)cc3)cc(-c3ccccc3)n2)cc1 ; c1ccc(-c2ccc(-c3nc(-c4ccccc4)nc(-c4cccc5c4cc(-c4ccccc4)c4c(-c6ccccc6)nn(-c6ccccc6)c45)n3)cc2)cc1]

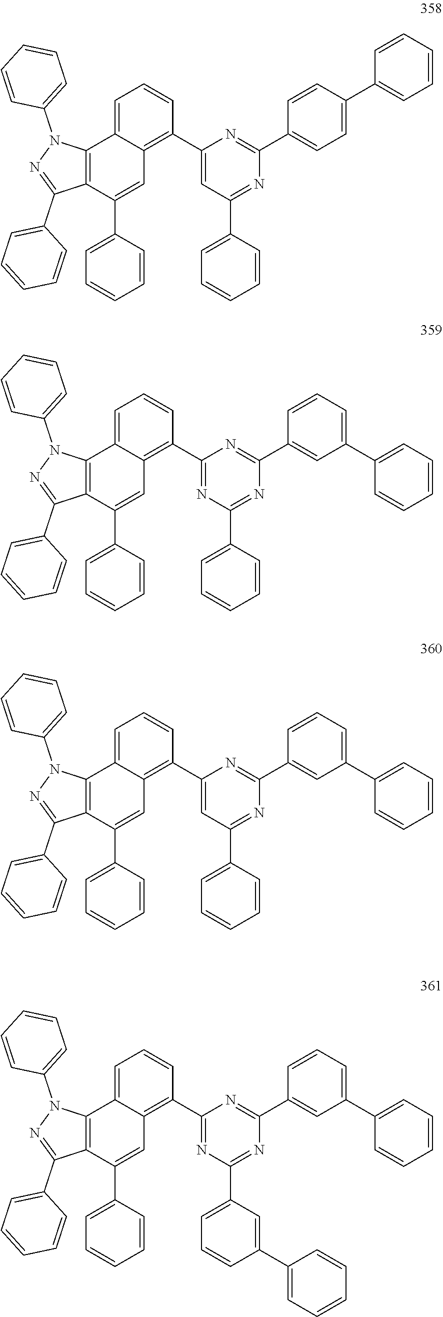 [SMILES: c1ccc(-c2ccc(-c3nc(-c4ccccc4)cc(-c4cccc5c4cc(-c4ccccc4)c4c(-c6ccccc6)nn(-c6ccccc6)c45)n3)cc2)cc1 ; c1ccc(-c2cccc(-c3nc(-c4ccccc4)nc(-c4cccc5c4cc(-c4ccccc4)c4c(-c6ccccc6)nn(-c6ccccc6)c45)n3)c2)cc1 ; c1ccc(-c2cccc(-c3nc(-c4ccccc4)cc(-c4cccc5c4cc(-c4ccccc4)c4c(-c6ccccc6)nn(-c6ccccc6)c45)n3)c2)cc1 ; c1ccc(-c2cccc(-c3nc(-c4cccc(-c5ccccc5)c4)nc(-c4cccc5c4cc(-c4ccccc4)c4c(-c6ccccc6)nn(-c6ccccc6)c45)n3)c2)cc1]

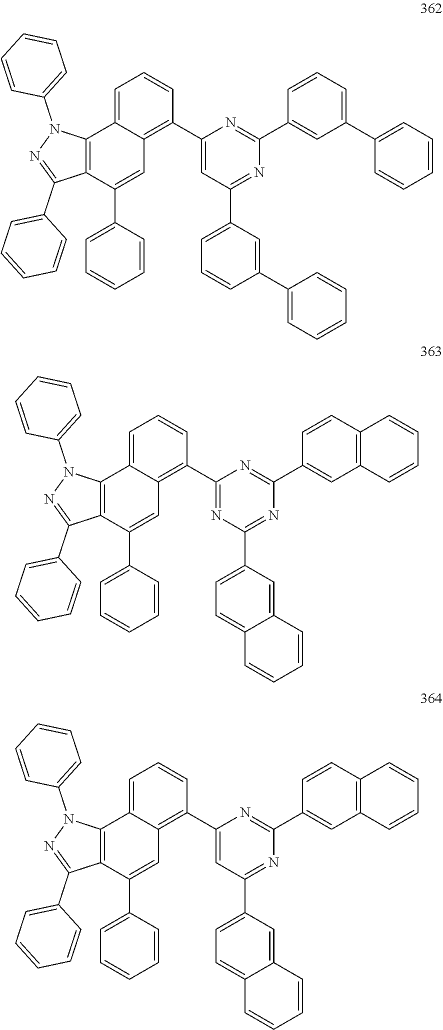 [SMILES: c1ccc(-c2cccc(-c3cc(-c4cccc5c4cc(-c4ccccc4)c4c(-c6ccccc6)nn(-c6ccccc6)c45)nc(-c4cccc(-c5ccccc5)c4)n3)c2)cc1 ; c1ccc(-c2cc3c(-c4nc(-c5ccc6ccccc6c5)nc(-c5ccc6ccccc6c5)n4)cccc3c3c2c(-c2ccccc2)nn3-c2ccccc2)cc1 ; c1ccc(-c2cc3c(-c4cc(-c5ccc6ccccc6c5)nc(-c5ccc6ccccc6c5)n4)cccc3c3c2c(-c2ccccc2)nn3-c2ccccc2)cc1]

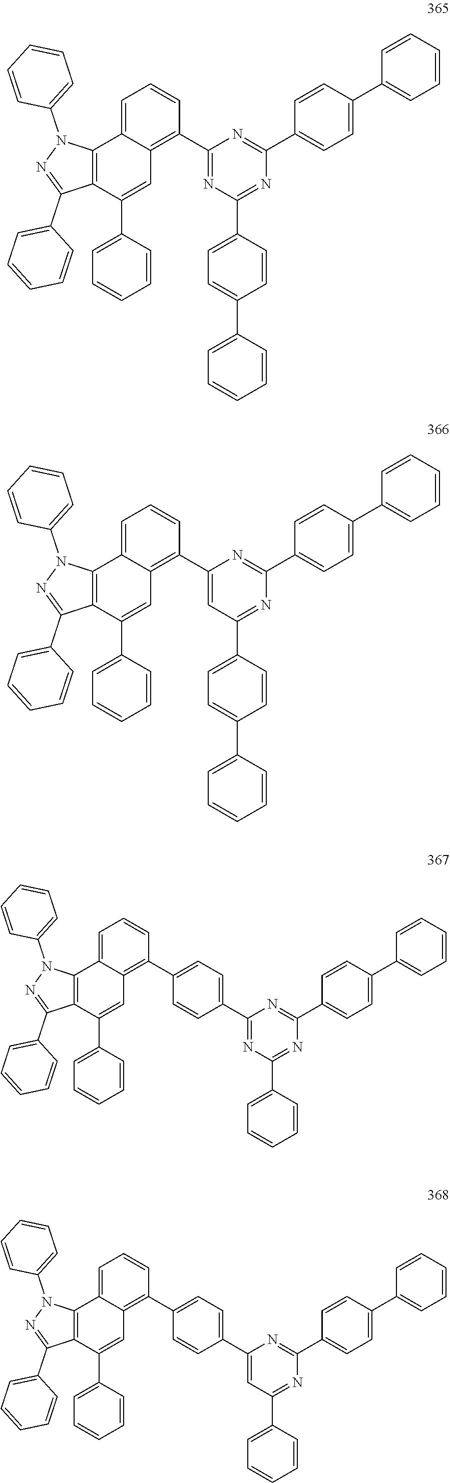 [SMILES: c1ccc(-c2ccc(-c3nc(-c4ccc(-c5ccccc5)cc4)nc(-c4cccc5c4cc(-c4ccccc4)c4c(-c6ccccc6)nn(-c6ccccc6)c45)n3)cc2)cc1 ; c1ccc(-c2ccc(-c3cc(-c4cccc5c4cc(-c4ccccc4)c4c(-c6ccccc6)nn(-c6ccccc6)c45)nc(-c4ccc(-c5ccccc5)cc4)n3)cc2)cc1 ; c1ccc(-c2ccc(-c3nc(-c4ccccc4)nc(-c4ccc(-c5cccc6c5cc(-c5ccccc5)c5c(-c7ccccc7)nn(-c7ccccc7)c56)cc4)n3)cc2)cc1 ; c1ccc(-c2ccc(-c3nc(-c4ccccc4)cc(-c4ccc(-c5cccc6c5cc(-c5ccccc5)c5c(-c7ccccc7)nn(-c7ccccc7)c56)cc4)n3)cc2)cc1]

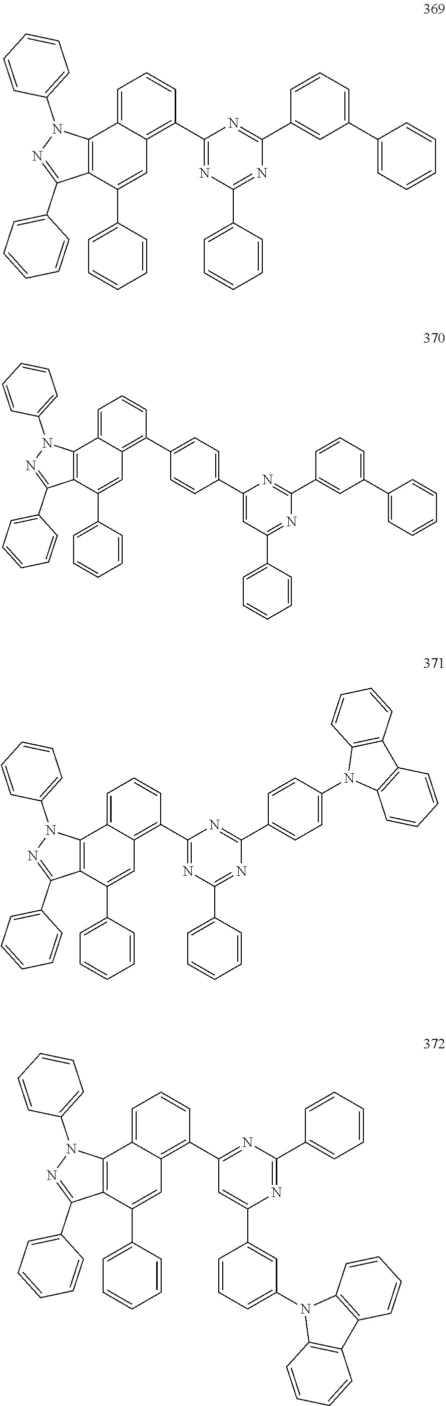 [SMILES: c1ccc(-c2cccc(-c3nc(-c4ccccc4)nc(-c4cccc5c4cc(-c4ccccc4)c4c(-c6ccccc6)nn(-c6ccccc6)c45)n3)c2)cc1 ; c1ccc(-c2cccc(-c3nc(-c4ccccc4)cc(-c4ccc(-c5cccc6c5cc(-c5ccccc5)c5c(-c7ccccc7)nn(-c7ccccc7)c56)cc4)n3)c2)cc1 ; c1ccc(-c2nc(-c3ccc(-n4c5ccccc5c5ccccc54)cc3)nc(-c3cccc4c3cc(-c3ccccc3)c3c(-c5ccccc5)nn(-c5ccccc5)c34)n2)cc1 ; c1ccc(-c2nc(-c3cccc(-n4c5ccccc5c5ccccc54)c3)cc(-c3cccc4c3cc(-c3ccccc3)c3c(-c5ccccc5)nn(-c5ccccc5)c34)n2)cc1]

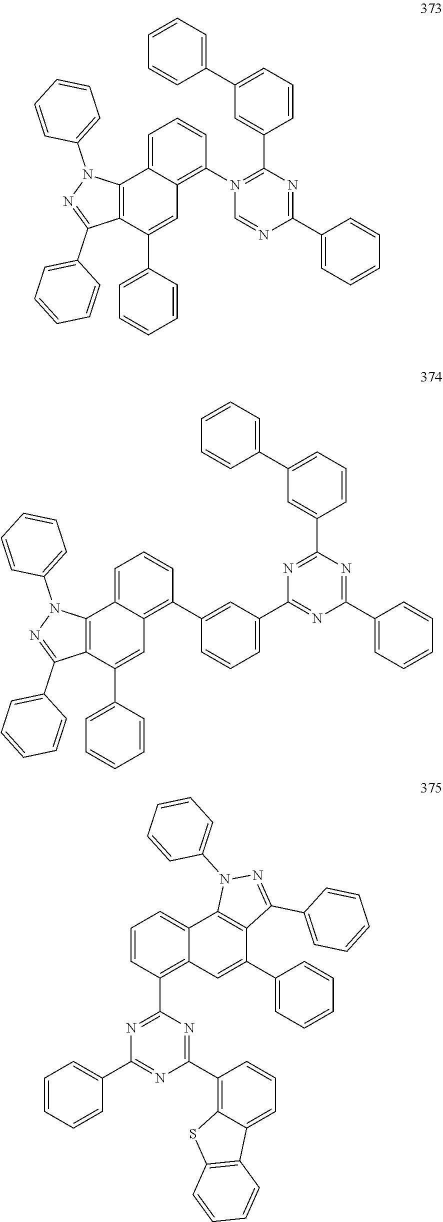

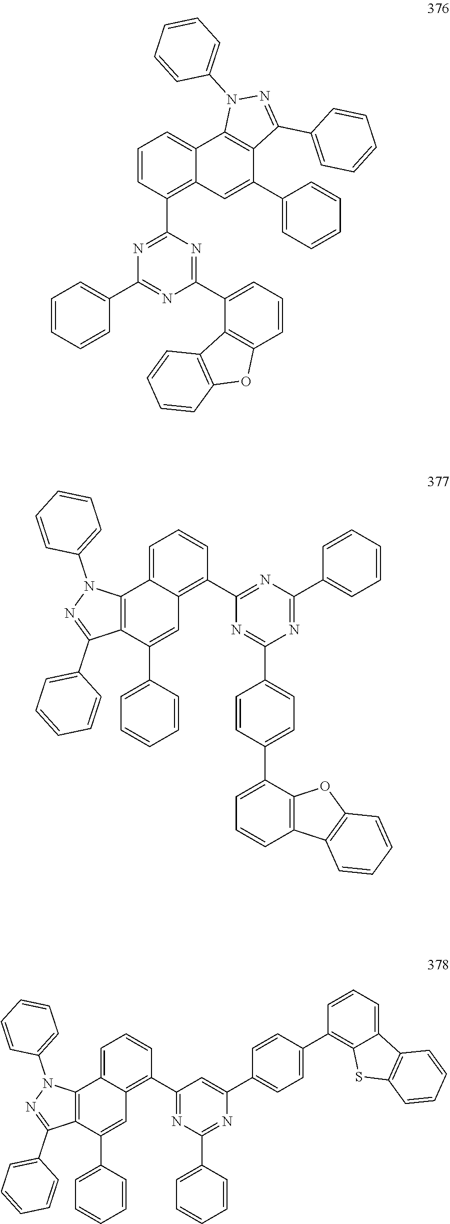 [SMILES: c1ccc(-c2nc(-c3cccc4c3cc(-c3ccccc3)c3c(-c5ccccc5)nn(-c5ccccc5)c34)nc(-c3cccc4oc5ccccc5c34)n2)cc1 ; c1ccc(-c2nc(-c3ccc(-c4cccc5c4oc4ccccc45)cc3)nc(-c3cccc4c3cc(-c3ccccc3)c3c(-c5ccccc5)nn(-c5ccccc5)c34)n2)cc1 ; c1ccc(-c2nc(-c3ccc(-c4cccc5c4sc4ccccc45)cc3)cc(-c3cccc4c3cc(-c3ccccc3)c3c(-c5ccccc5)nn(-c5ccccc5)c34)n2)cc1]

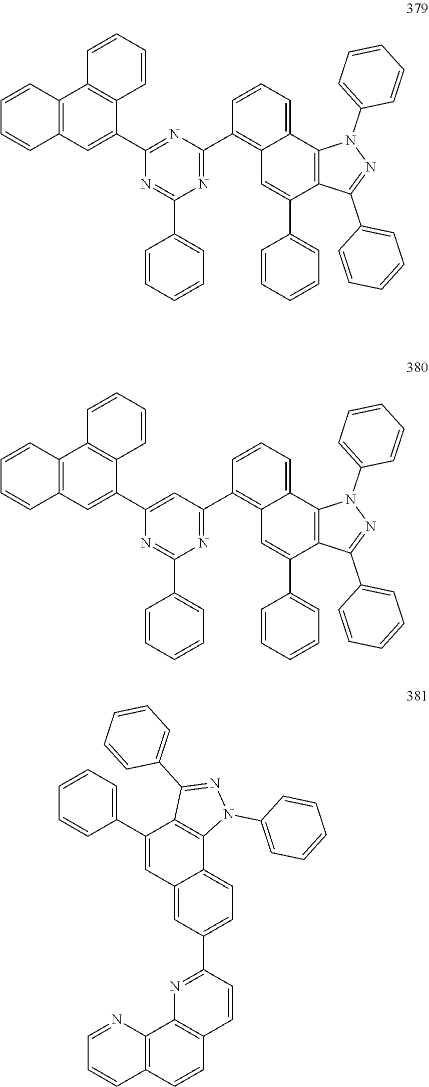 [SMILES: c1ccc(-c2nc(-c3cc4ccccc4c4ccccc34)nc(-c3cccc4c3cc(-c3ccccc3)c3c(-c5ccccc5)nn(-c5ccccc5)c34)n2)cc1 ; c1ccc(-c2nc(-c3cc4ccccc4c4ccccc34)cc(-c3cccc4c3cc(-c3ccccc3)c3c(-c5ccccc5)nn(-c5ccccc5)c34)n2)cc1 ; c1ccc(-c2cc3cc(-c4ccc5ccc6cccnc6c5n4)ccc3c3c2c(-c2ccccc2)nn3-c2ccccc2)cc1]

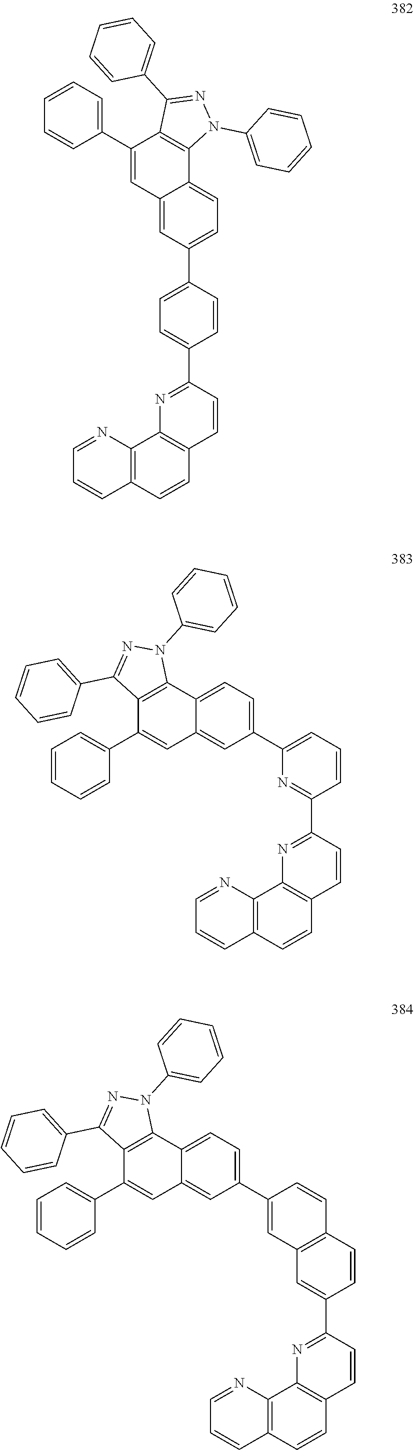 [SMILES: c1ccc(-c2cc3cc(-c4ccc(-c5ccc6ccc7cccnc7c6n5)cc4)ccc3c3c2c(-c2ccccc2)nn3-c2ccccc2)cc1 ; c1ccc(-c2cc3cc(-c4cccc(-c5ccc6ccc7cccnc7c6n5)n4)ccc3c3c2c(-c2ccccc2)nn3-c2ccccc2)cc1 ; c1ccc(-c2cc3cc(-c4ccc5ccc(-c6ccc7ccc8cccnc8c7n6)cc5c4)ccc3c3c2c(-c2ccccc2)nn3-c2ccccc2)cc1]

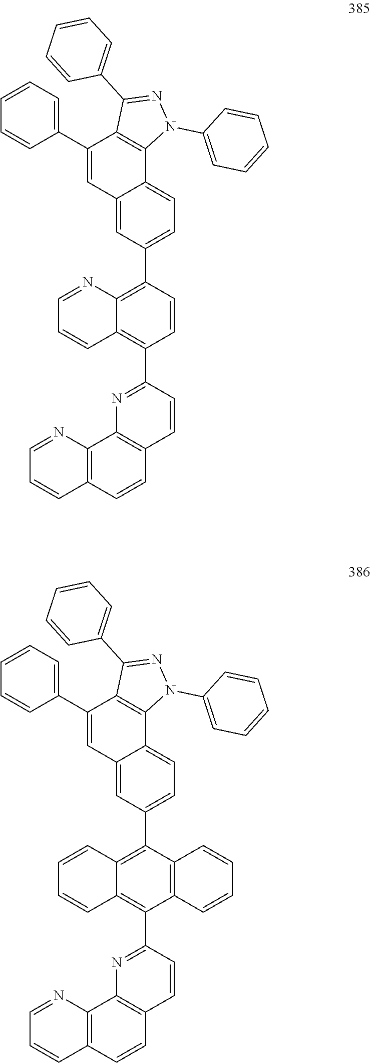 [SMILES: c1ccc(-c2cc3cc(-c4ccc(-c5ccc6ccc7cccnc7c6n5)c5cccnc45)ccc3c3c2c(-c2ccccc2)nn3-c2ccccc2)cc1 ; c1ccc(-c2cc3cc(-c4c5ccccc5c(-c5ccc6ccc7cccnc7c6n5)c5ccccc45)ccc3c3c2c(-c2ccccc2)nn3-c2ccccc2)cc1]

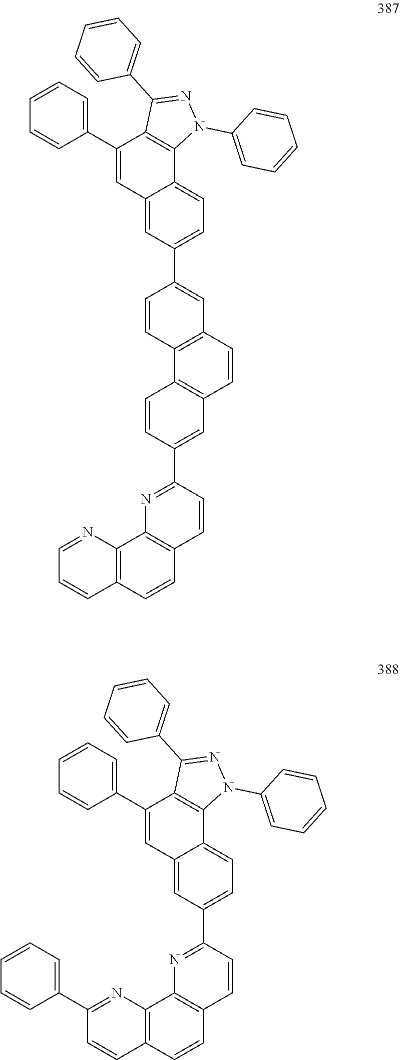 [SMILES: c1ccc(-c2cc3cc(-c4ccc5c(ccc6cc(-c7ccc8ccc9cccnc9c8n7)ccc65)c4)ccc3c3c2c(-c2ccccc2)nn3-c2ccccc2)cc1 ; c1ccc(-c2ccc3ccc4ccc(-c5ccc6c(c5)cc(-c5ccccc5)c5c(-c7ccccc7)nn(-c7ccccc7)c56)nc4c3n2)cc1]

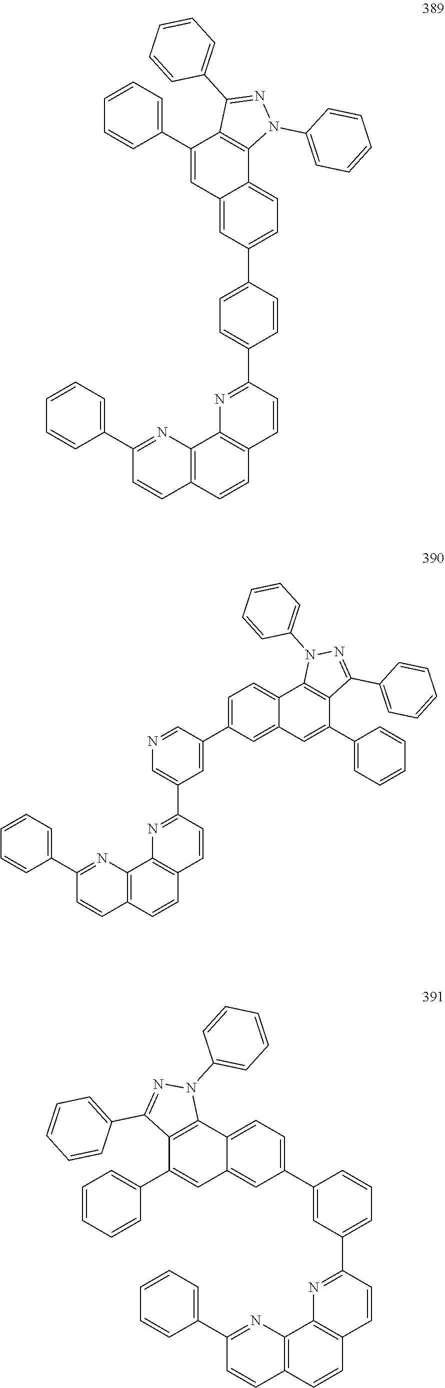 [SMILES: c1ccc(-c2ccc3ccc4ccc(-c5ccc(-c6ccc7c(c6)cc(-c6ccccc6)c6c(-c8ccccc8)nn(-c8ccccc8)c67)cc5)nc4c3n2)cc1 ; c1ccc(-c2ccc3ccc4ccc(-c5cncc(-c6ccc7c(c6)cc(-c6ccccc6)c6c(-c8ccccc8)nn(-c8ccccc8)c67)c5)nc4c3n2)cc1 ; c1ccc(-c2ccc3ccc4ccc(-c5cccc(-c6ccc7c(c6)cc(-c6ccccc6)c6c(-c8ccccc8)nn(-c8ccccc8)c67)c5)nc4c3n2)cc1]

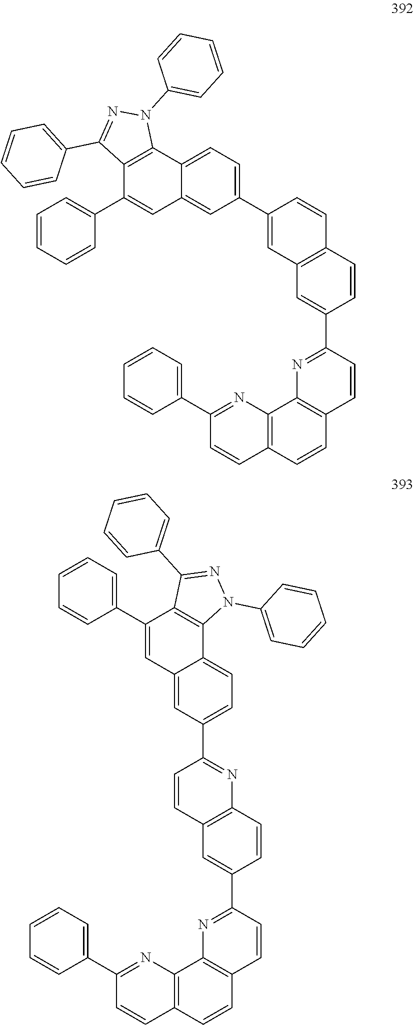 [SMILES: c1ccc(-c2ccc3ccc4ccc(-c5ccc6ccc(-c7ccc8c(c7)cc(-c7ccccc7)c7c(-c9ccccc9)nn(-c9ccccc9)c78)cc6c5)nc4c3n2)cc1 ; c1ccc(-c2ccc3ccc4ccc(-c5ccc6nc(-c7ccc8c(c7)cc(-c7ccccc7)c7c(-c9ccccc9)nn(-c9ccccc9)c78)ccc6c5)nc4c3n2)cc1]

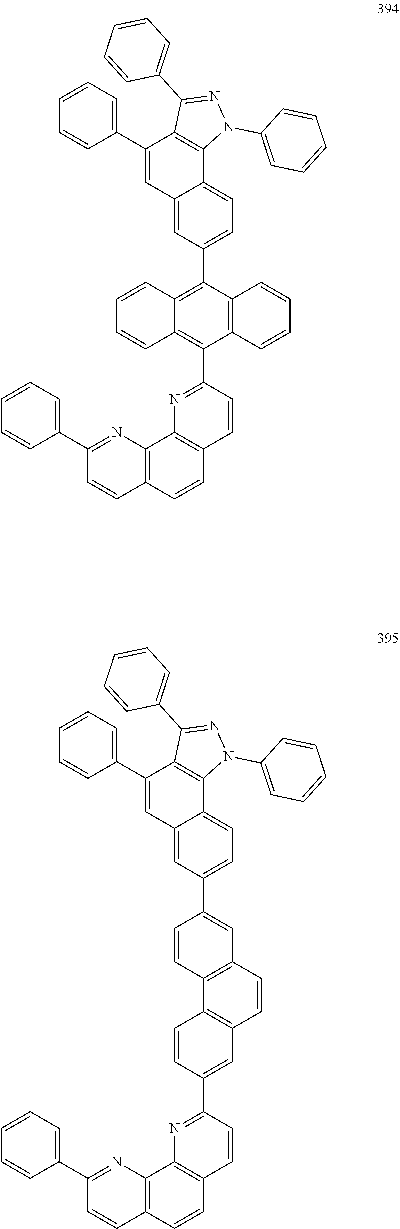 [SMILES: c1ccc(-c2ccc3ccc4ccc(-c5c6ccccc6c(-c6ccc7c(c6)cc(-c6ccccc6)c6c(-c8ccccc8)nn(-c8ccccc8)c67)c6ccccc56)nc4c3n2)cc1 ; c1ccc(-c2ccc3ccc4ccc(-c5ccc6c(ccc7cc(-c8ccc9c(c8)cc(-c8ccccc8)c8c(-c%10ccccc%10)nn(-c%10ccccc%10)c89)ccc76)c5)nc4c3n2)cc1]

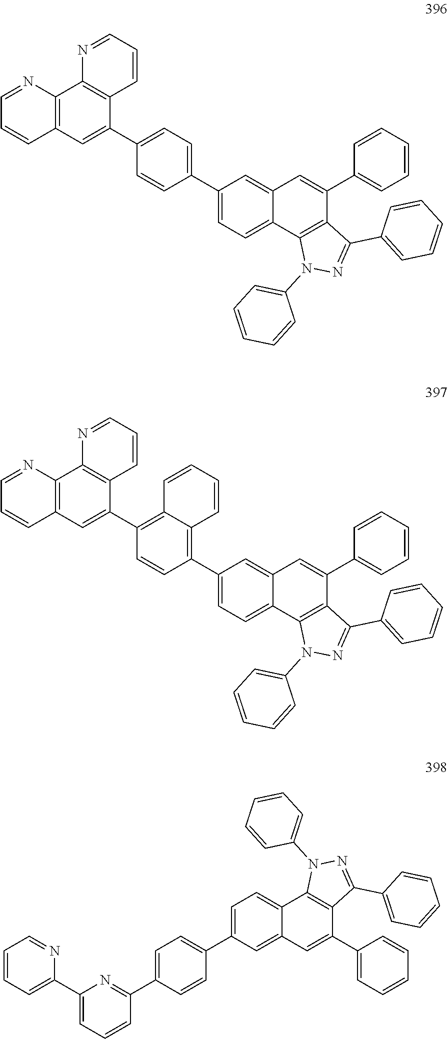 [SMILES: c1ccc(-c2cc3cc(-c4ccc(-c5cc6cccnc6c6ncccc56)cc4)ccc3c3c2c(-c2ccccc2)nn3-c2ccccc2)cc1 ; c1ccc(-c2cc3cc(-c4ccc(-c5cc6cccnc6c6ncccc56)c5ccccc45)ccc3c3c2c(-c2ccccc2)nn3-c2ccccc2)cc1 ; c1ccc(-c2cc3cc(-c4ccc(-c5cccc(-c6ccccn6)n5)cc4)ccc3c3c2c(-c2ccccc2)nn3-c2ccccc2)cc1]

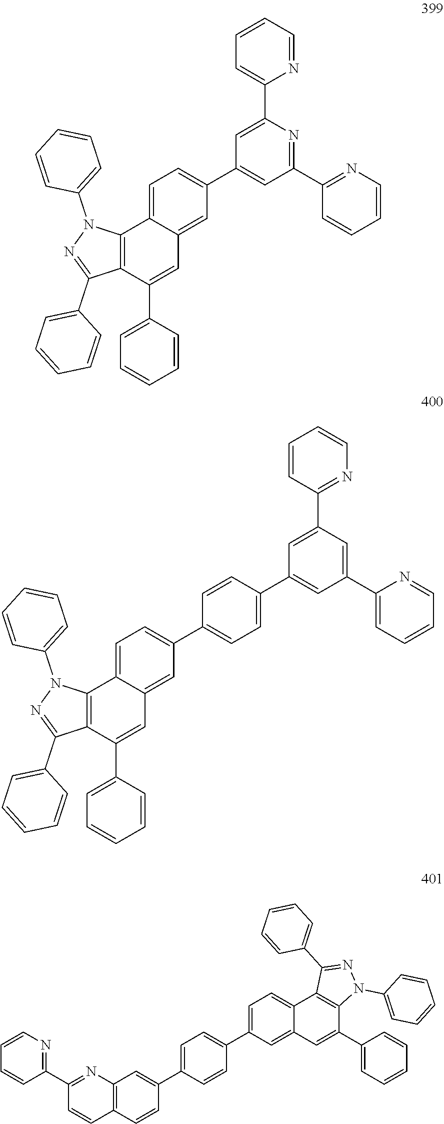 [SMILES: c1ccc(-c2cc3cc(-c4cc(-c5ccccn5)nc(-c5ccccn5)c4)ccc3c3c2c(-c2ccccc2)nn3-c2ccccc2)cc1 ; c1ccc(-c2cc3cc(-c4ccc(-c5cc(-c6ccccn6)cc(-c6ccccn6)c5)cc4)ccc3c3c2c(-c2ccccc2)nn3-c2ccccc2)cc1 ; c1ccc(-c2nn(-c3ccccc3)c3c(-c4ccccc4)cc4cc(-c5ccc(-c6ccc7ccc(-c8ccccn8)nc7c6)cc5)ccc4c23)cc1]

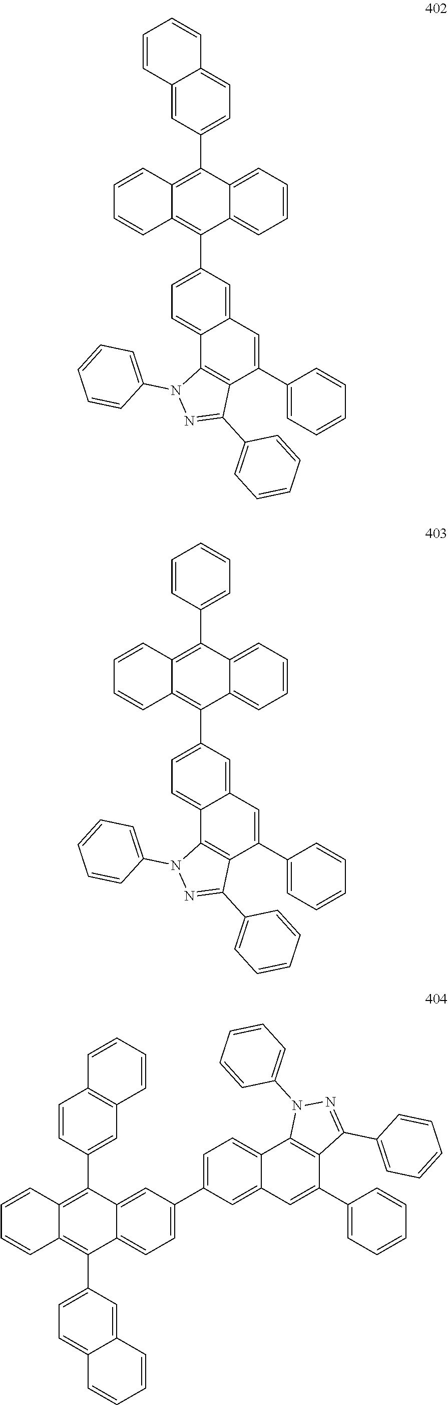 [SMILES: c1ccc(-c2cc3cc(-c4c5ccccc5c(-c5ccc6ccccc6c5)c5ccccc45)ccc3c3c2c(-c2ccccc2)nn3-c2ccccc2)cc1 ; c1ccc(-c2c3ccccc3c(-c3ccc4c(c3)cc(-c3ccccc3)c3c(-c5ccccc5)nn(-c5ccccc5)c34)c3ccccc23)cc1 ; c1ccc(-c2cc3cc(-c4ccc5c(-c6ccc7ccccc7c6)c6ccccc6c(-c6ccc7ccccc7c6)c5c4)ccc3c3c2c(-c2ccccc2)nn3-c2ccccc2)cc1]

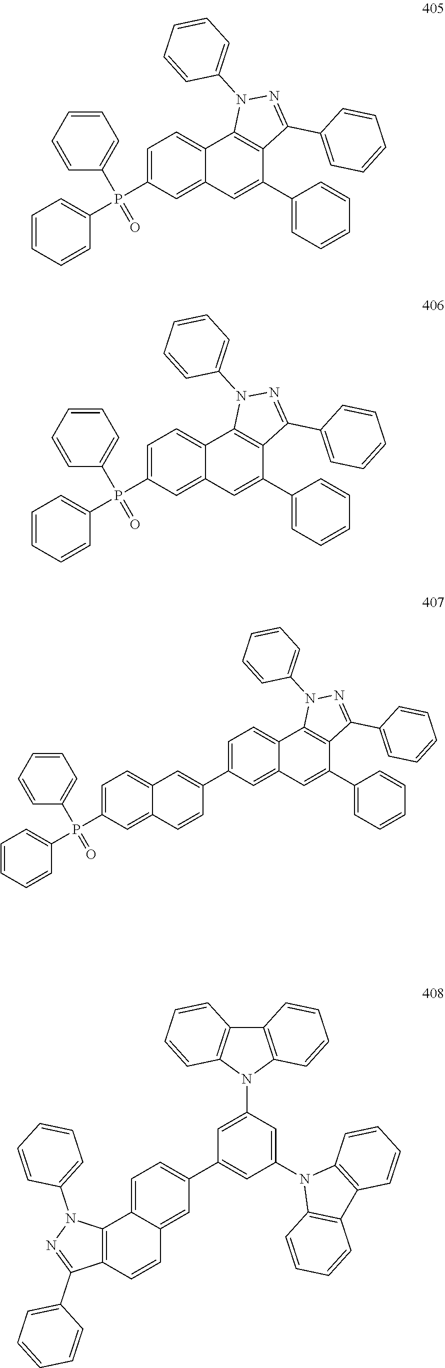 [SMILES: O=P(c1ccccc1)(c1ccccc1)c1ccc2c(c1)cc(-c1ccccc1)c1c(-c3ccccc3)nn(-c3ccccc3)c12 ; O=P(c1ccccc1)(c1ccccc1)c1ccc2c(c1)cc(-c1ccccc1)c1c(-c3ccccc3)nn(-c3ccccc3)c12 ; O=P(c1ccccc1)(c1ccccc1)c1ccc2cc(-c3ccc4c(c3)cc(-c3ccccc3)c3c(-c5ccccc5)nn(-c5ccccc5)c34)ccc2c1 ; c1ccc(-c2nn(-c3ccccc3)c3c2ccc2cc(-c4cc(-n5c6ccccc6c6ccccc65)cc(-n5c6ccccc6c6ccccc65)c4)ccc23)cc1]

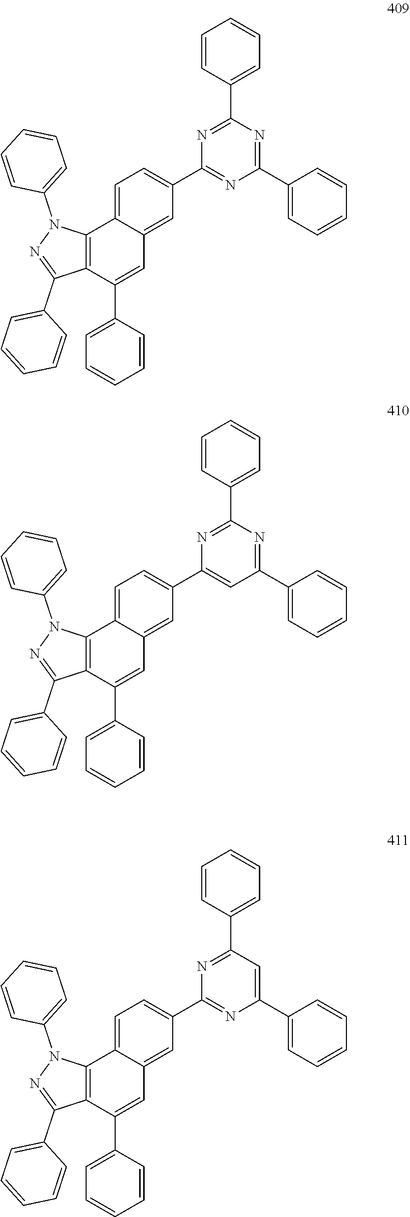 [SMILES: c1ccc(-c2nc(-c3ccccc3)nc(-c3ccc4c(c3)cc(-c3ccccc3)c3c(-c5ccccc5)nn(-c5ccccc5)c34)n2)cc1 ; c1ccc(-c2cc(-c3ccc4c(c3)cc(-c3ccccc3)c3c(-c5ccccc5)nn(-c5ccccc5)c34)nc(-c3ccccc3)n2)cc1 ; c1ccc(-c2cc(-c3ccccc3)nc(-c3ccc4c(c3)cc(-c3ccccc3)c3c(-c5ccccc5)nn(-c5ccccc5)c34)n2)cc1]

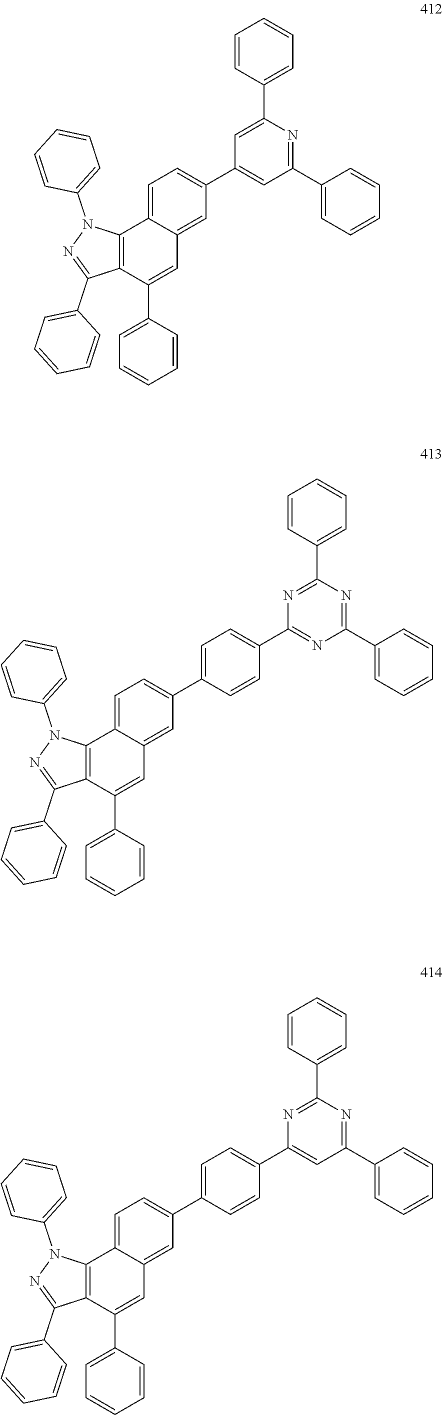 [SMILES: c1ccc(-c2cc(-c3ccc4c(c3)cc(-c3ccccc3)c3c(-c5ccccc5)nn(-c5ccccc5)c34)cc(-c3ccccc3)n2)cc1 ; c1ccc(-c2nc(-c3ccccc3)nc(-c3ccc(-c4ccc5c(c4)cc(-c4ccccc4)c4c(-c6ccccc6)nn(-c6ccccc6)c45)cc3)n2)cc1 ; c1ccc(-c2cc(-c3ccc(-c4ccc5c(c4)cc(-c4ccccc4)c4c(-c6ccccc6)nn(-c6ccccc6)c45)cc3)nc(-c3ccccc3)n2)cc1]

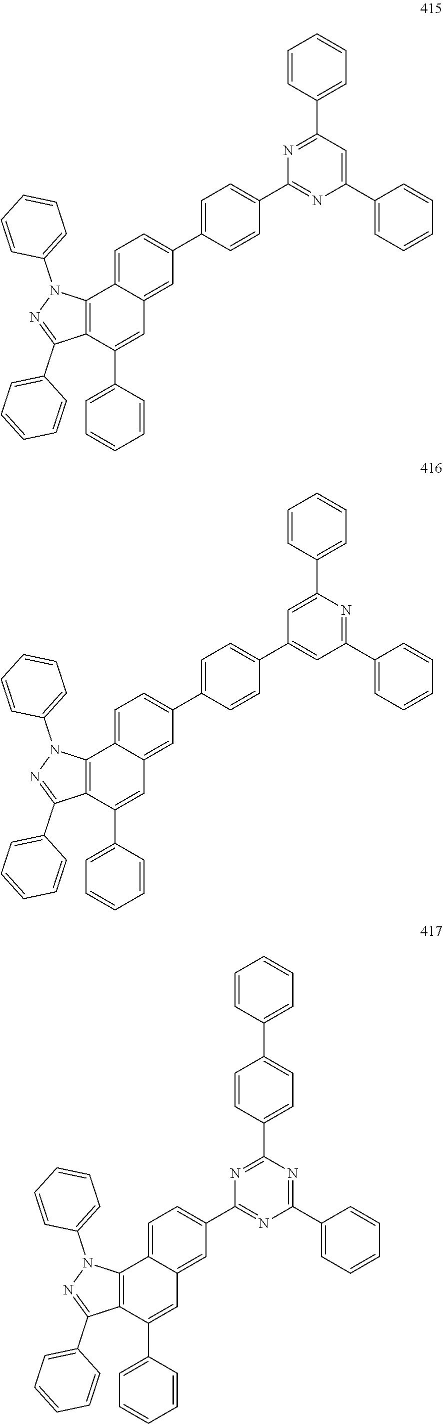 [SMILES: c1ccc(-c2cc(-c3ccccc3)nc(-c3ccc(-c4ccc5c(c4)cc(-c4ccccc4)c4c(-c6ccccc6)nn(-c6ccccc6)c45)cc3)n2)cc1 ; c1ccc(-c2cc(-c3ccc(-c4ccc5c(c4)cc(-c4ccccc4)c4c(-c6ccccc6)nn(-c6ccccc6)c45)cc3)cc(-c3ccccc3)n2)cc1 ; c1ccc(-c2ccc(-c3nc(-c4ccccc4)nc(-c4ccc5c(c4)cc(-c4ccccc4)c4c(-c6ccccc6)nn(-c6ccccc6)c45)n3)cc2)cc1]

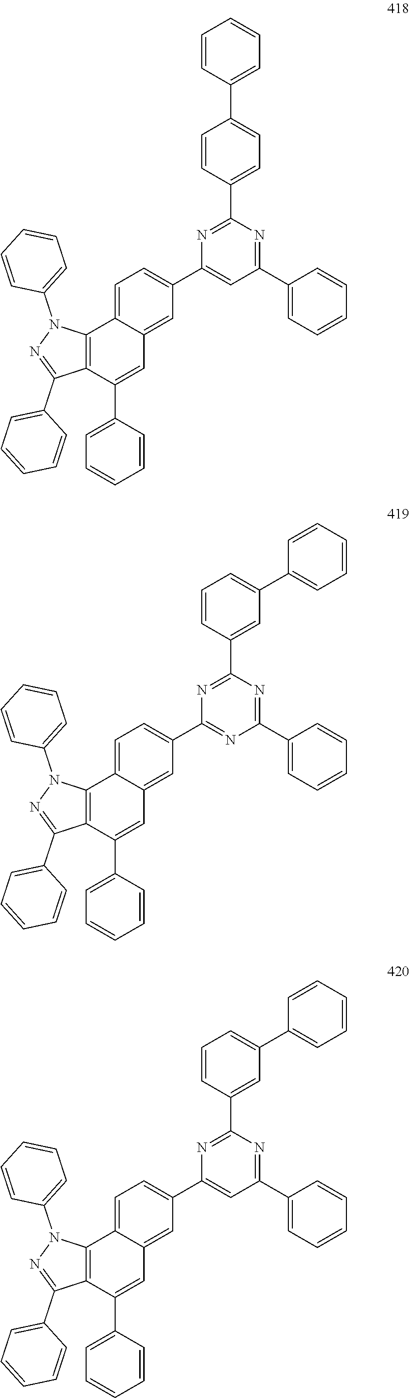 [SMILES: c1ccc(-c2ccc(-c3nc(-c4ccccc4)cc(-c4ccc5c(c4)cc(-c4ccccc4)c4c(-c6ccccc6)nn(-c6ccccc6)c45)n3)cc2)cc1 ; c1ccc(-c2cccc(-c3nc(-c4ccccc4)nc(-c4ccc5c(c4)cc(-c4ccccc4)c4c(-c6ccccc6)nn(-c6ccccc6)c45)n3)c2)cc1 ; c1ccc(-c2cccc(-c3nc(-c4ccccc4)cc(-c4ccc5c(c4)cc(-c4ccccc4)c4c(-c6ccccc6)nn(-c6ccccc6)c45)n3)c2)cc1]

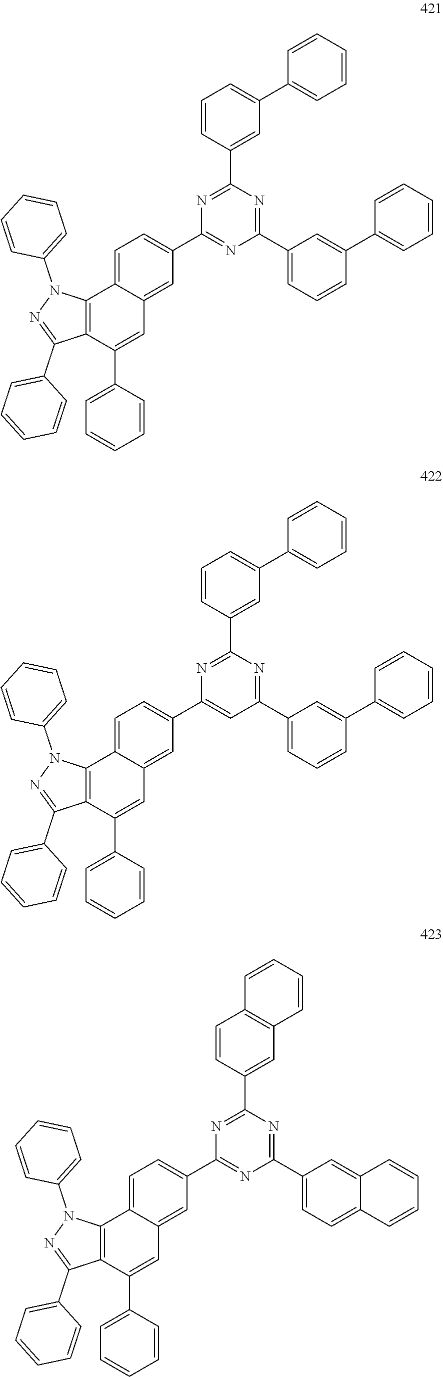 [SMILES: c1ccc(-c2cccc(-c3nc(-c4cccc(-c5ccccc5)c4)nc(-c4ccc5c(c4)cc(-c4ccccc4)c4c(-c6ccccc6)nn(-c6ccccc6)c45)n3)c2)cc1 ; c1ccc(-c2cccc(-c3cc(-c4ccc5c(c4)cc(-c4ccccc4)c4c(-c6ccccc6)nn(-c6ccccc6)c45)nc(-c4cccc(-c5ccccc5)c4)n3)c2)cc1 ; c1ccc(-c2cc3cc(-c4nc(-c5ccc6ccccc6c5)nc(-c5ccc6ccccc6c5)n4)ccc3c3c2c(-c2ccccc2)nn3-c2ccccc2)cc1]

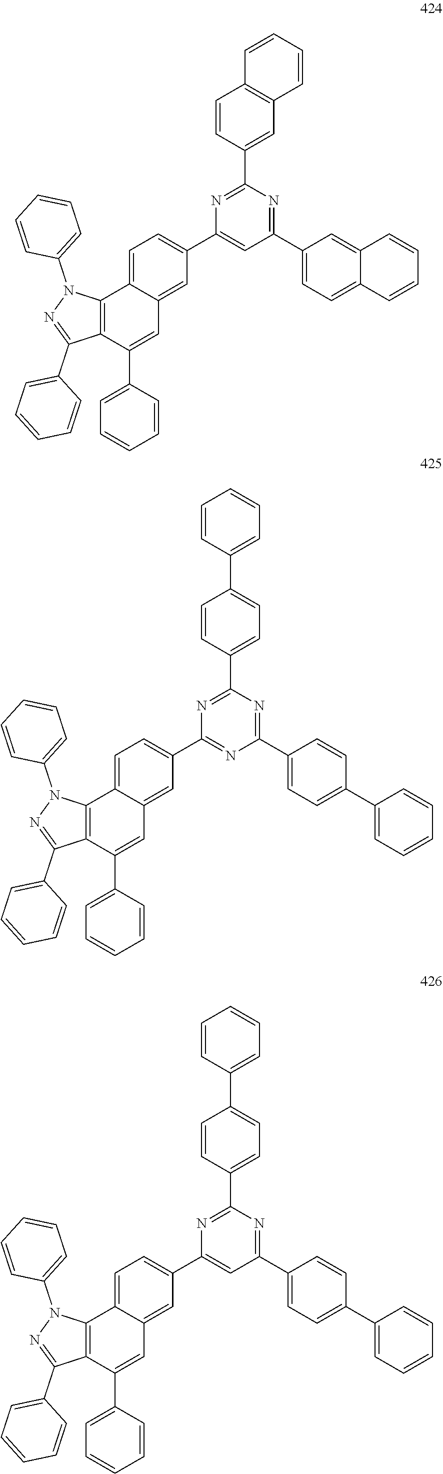 [SMILES: c1ccc(-c2cc3cc(-c4cc(-c5ccc6ccccc6c5)nc(-c5ccc6ccccc6c5)n4)ccc3c3c2c(-c2ccccc2)nn3-c2ccccc2)cc1 ; c1ccc(-c2ccc(-c3nc(-c4ccc(-c5ccccc5)cc4)nc(-c4ccc5c(c4)cc(-c4ccccc4)c4c(-c6ccccc6)nn(-c6ccccc6)c45)n3)cc2)cc1 ; c1ccc(-c2ccc(-c3cc(-c4ccc5c(c4)cc(-c4ccccc4)c4c(-c6ccccc6)nn(-c6ccccc6)c45)nc(-c4ccc(-c5ccccc5)cc4)n3)cc2)cc1]

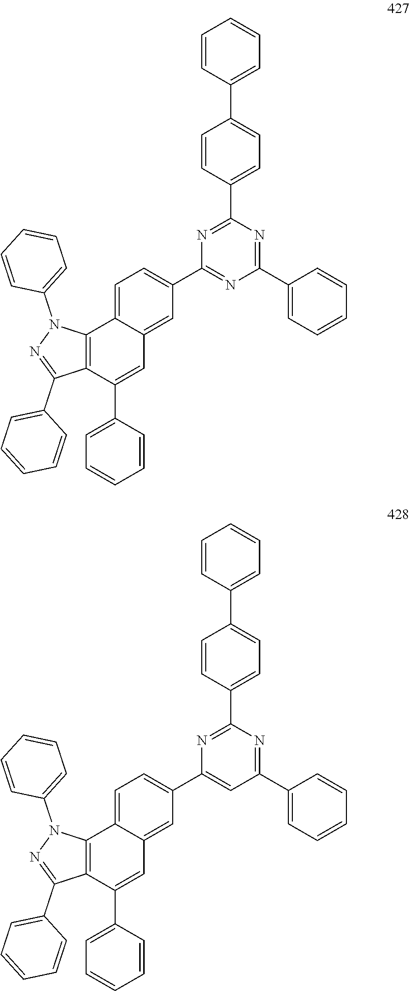 [SMILES: c1ccc(-c2ccc(-c3nc(-c4ccccc4)nc(-c4ccc5c(c4)cc(-c4ccccc4)c4c(-c6ccccc6)nn(-c6ccccc6)c45)n3)cc2)cc1 ; c1ccc(-c2ccc(-c3nc(-c4ccccc4)cc(-c4ccc5c(c4)cc(-c4ccccc4)c4c(-c6ccccc6)nn(-c6ccccc6)c45)n3)cc2)cc1]

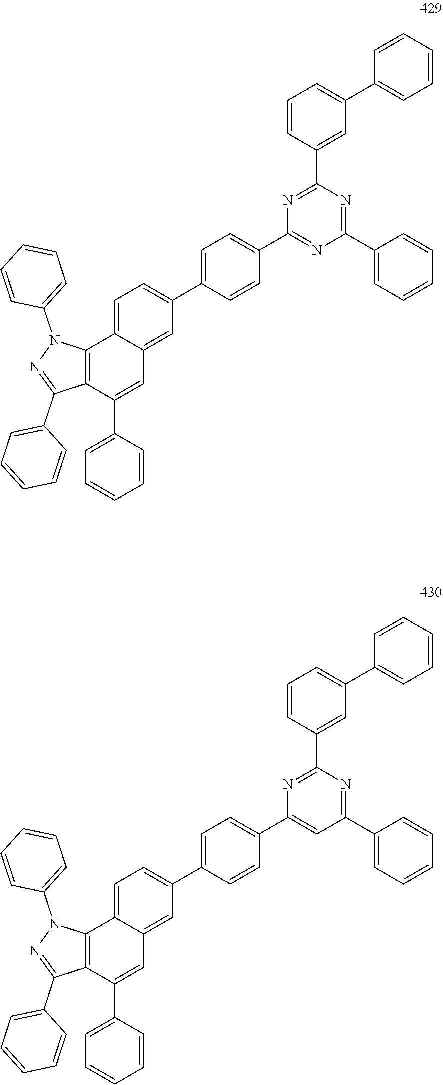 [SMILES: c1ccc(-c2cccc(-c3nc(-c4ccccc4)nc(-c4ccc(-c5ccc6c(c5)cc(-c5ccccc5)c5c(-c7ccccc7)nn(-c7ccccc7)c56)cc4)n3)c2)cc1 ; c1ccc(-c2cccc(-c3nc(-c4ccccc4)cc(-c4ccc(-c5ccc6c(c5)cc(-c5ccccc5)c5c(-c7ccccc7)nn(-c7ccccc7)c56)cc4)n3)c2)cc1]

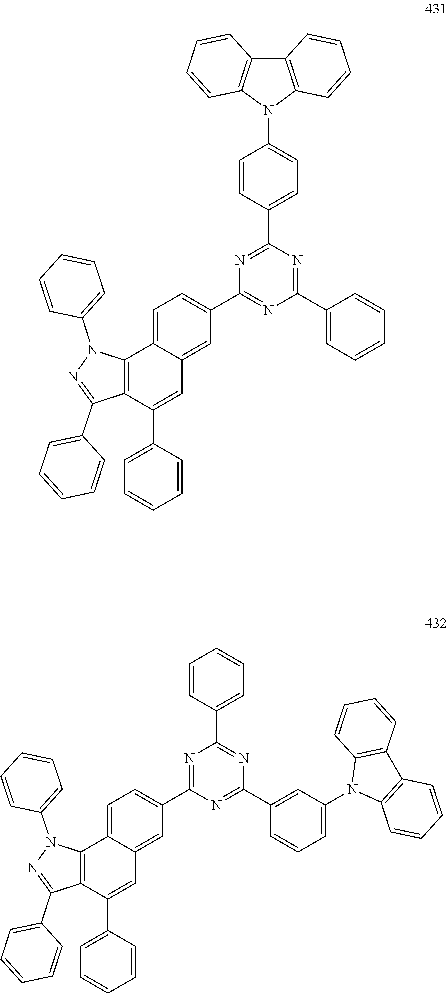 [SMILES: c1ccc(-c2nc(-c3ccc(-n4c5ccccc5c5ccccc54)cc3)nc(-c3ccc4c(c3)cc(-c3ccccc3)c3c(-c5ccccc5)nn(-c5ccccc5)c34)n2)cc1 ; c1ccc(-c2nc(-c3cccc(-n4c5ccccc5c5ccccc54)c3)nc(-c3ccc4c(c3)cc(-c3ccccc3)c3c(-c5ccccc5)nn(-c5ccccc5)c34)n2)cc1]

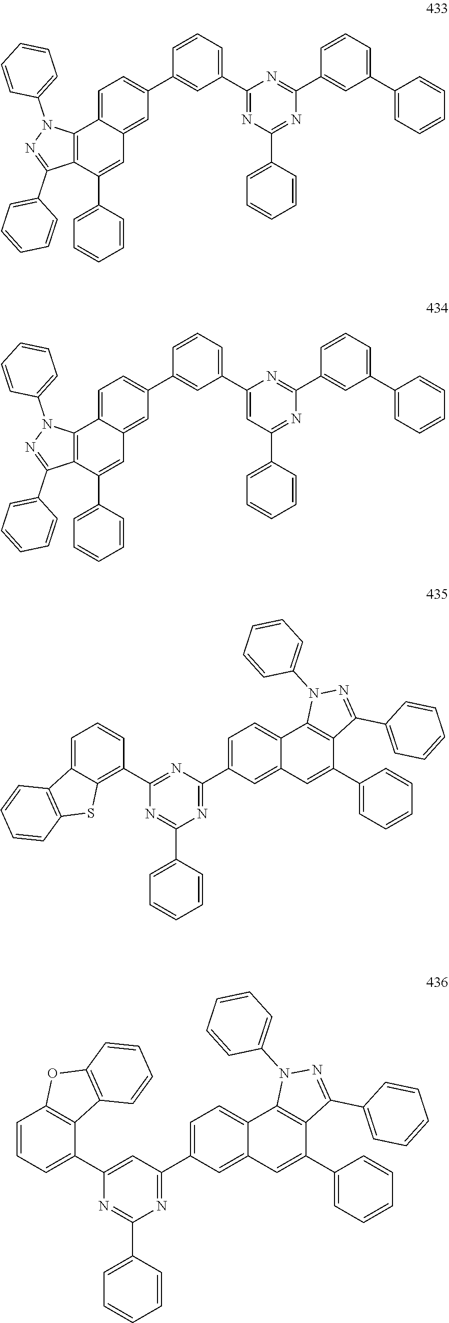 [SMILES: c1ccc(-c2cccc(-c3nc(-c4ccccc4)nc(-c4cccc(-c5ccc6c(c5)cc(-c5ccccc5)c5c(-c7ccccc7)nn(-c7ccccc7)c56)c4)n3)c2)cc1 ; c1ccc(-c2cccc(-c3nc(-c4ccccc4)cc(-c4cccc(-c5ccc6c(c5)cc(-c5ccccc5)c5c(-c7ccccc7)nn(-c7ccccc7)c56)c4)n3)c2)cc1 ; c1ccc(-c2nc(-c3ccc4c(c3)cc(-c3ccccc3)c3c(-c5ccccc5)nn(-c5ccccc5)c34)nc(-c3cccc4c3sc3ccccc34)n2)cc1 ; c1ccc(-c2nc(-c3ccc4c(c3)cc(-c3ccccc3)c3c(-c5ccccc5)nn(-c5ccccc5)c34)cc(-c3cccc4oc5ccccc5c34)n2)cc1]

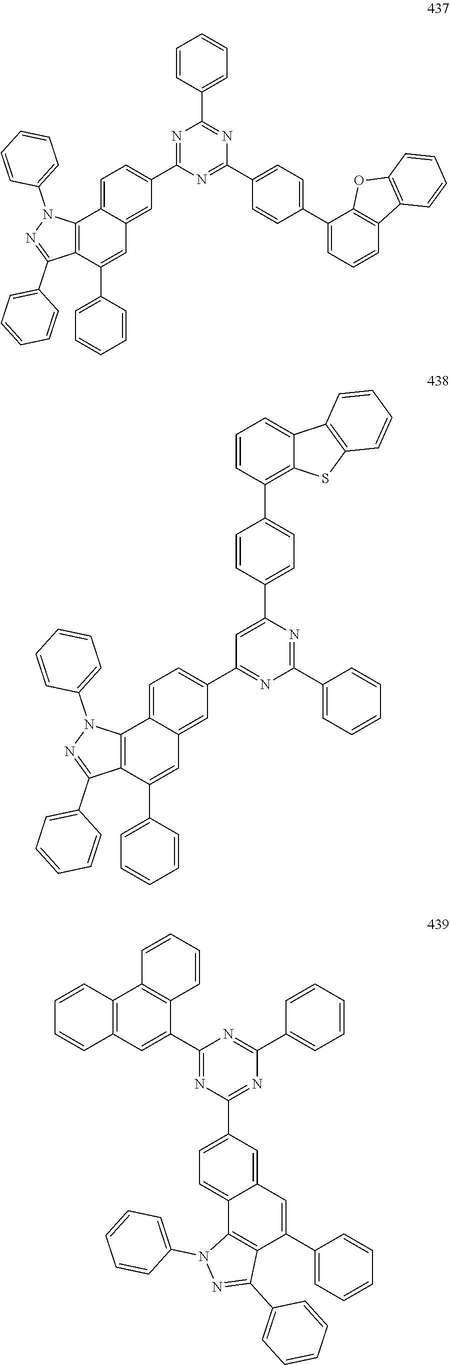 [SMILES: c1ccc(-c2nc(-c3ccc(-c4cccc5c4oc4ccccc45)cc3)nc(-c3ccc4c(c3)cc(-c3ccccc3)c3c(-c5ccccc5)nn(-c5ccccc5)c34)n2)cc1 ; c1ccc(-c2nc(-c3ccc(-c4cccc5c4sc4ccccc45)cc3)cc(-c3ccc4c(c3)cc(-c3ccccc3)c3c(-c5ccccc5)nn(-c5ccccc5)c34)n2)cc1 ; c1ccc(-c2nc(-c3ccc4c(c3)cc(-c3ccccc3)c3c(-c5ccccc5)nn(-c5ccccc5)c34)nc(-c3cc4ccccc4c4ccccc34)n2)cc1]

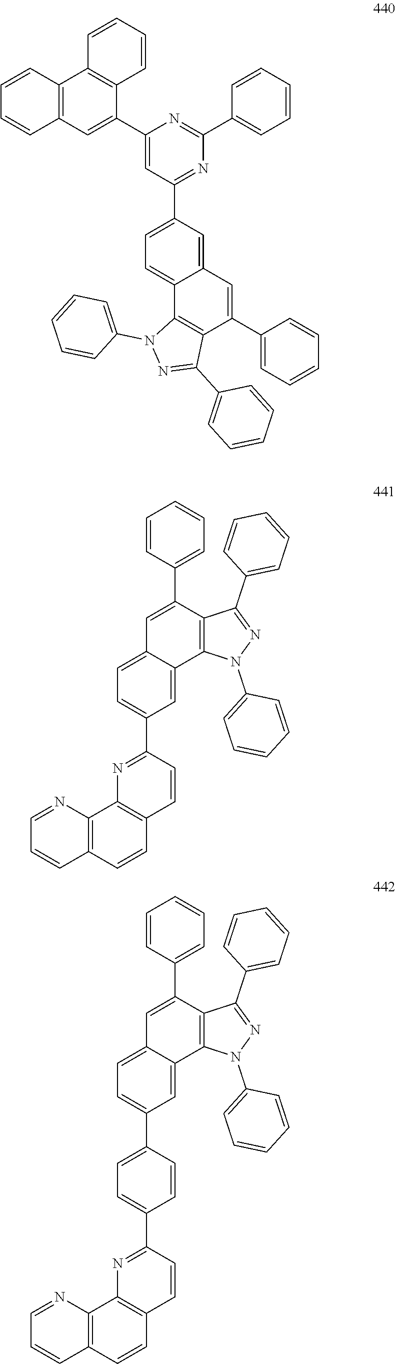 [SMILES: c1ccc(-c2nc(-c3ccc4c(c3)cc(-c3ccccc3)c3c(-c5ccccc5)nn(-c5ccccc5)c34)cc(-c3cc4ccccc4c4ccccc34)n2)cc1 ; c1ccc(-c2cc3ccc(-c4ccc5ccc6cccnc6c5n4)cc3c3c2c(-c2ccccc2)nn3-c2ccccc2)cc1 ; c1ccc(-c2cc3ccc(-c4ccc(-c5ccc6ccc7cccnc7c6n5)cc4)cc3c3c2c(-c2ccccc2)nn3-c2ccccc2)cc1]

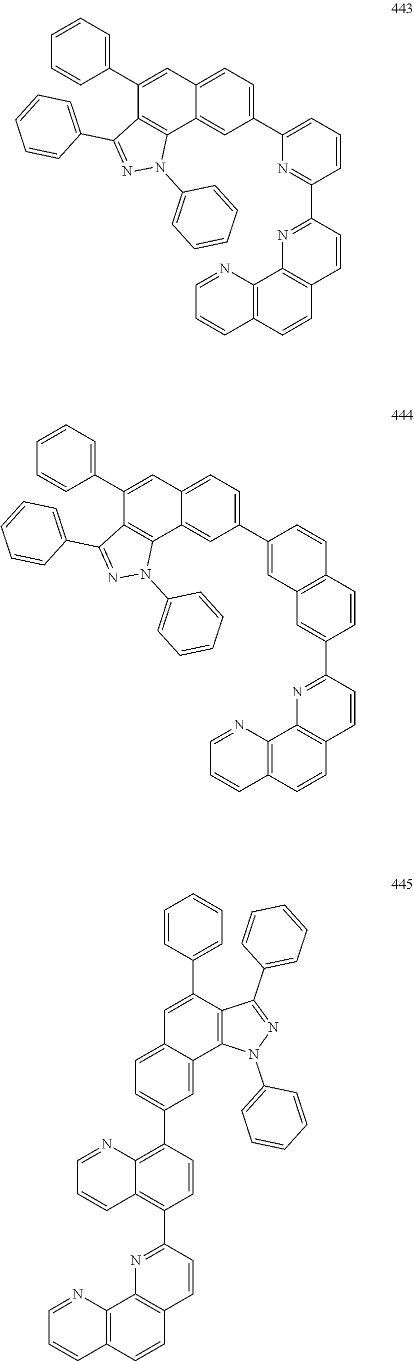 [SMILES: c1ccc(-c2cc3ccc(-c4cccc(-c5ccc6ccc7cccnc7c6n5)n4)cc3c3c2c(-c2ccccc2)nn3-c2ccccc2)cc1 ; c1ccc(-c2cc3ccc(-c4ccc5ccc(-c6ccc7ccc8cccnc8c7n6)cc5c4)cc3c3c2c(-c2ccccc2)nn3-c2ccccc2)cc1 ; c1ccc(-c2cc3ccc(-c4ccc(-c5ccc6ccc7cccnc7c6n5)c5cccnc45)cc3c3c2c(-c2ccccc2)nn3-c2ccccc2)cc1]

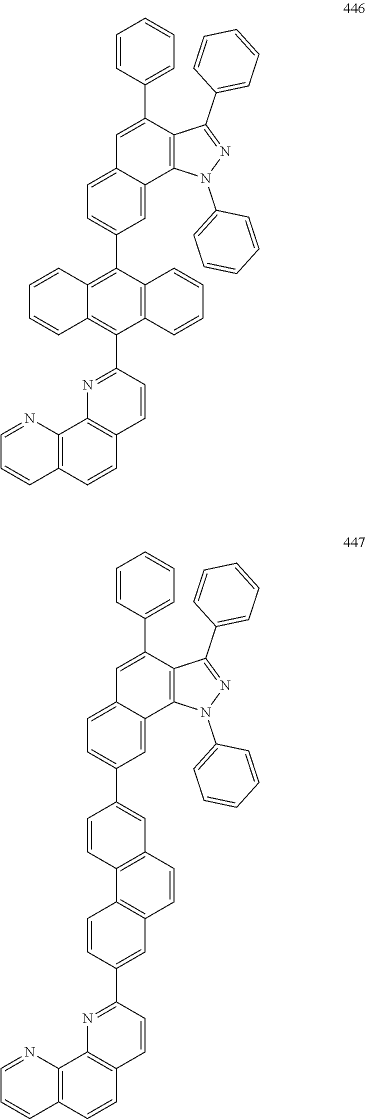 [SMILES: c1ccc(-c2cc3ccc(-c4c5ccccc5c(-c5ccc6ccc7cccnc7c6n5)c5ccccc45)cc3c3c2c(-c2ccccc2)nn3-c2ccccc2)cc1 ; c1ccc(-c2cc3ccc(-c4ccc5c(ccc6cc(-c7ccc8ccc9cccnc9c8n7)ccc65)c4)cc3c3c2c(-c2ccccc2)nn3-c2ccccc2)cc1]

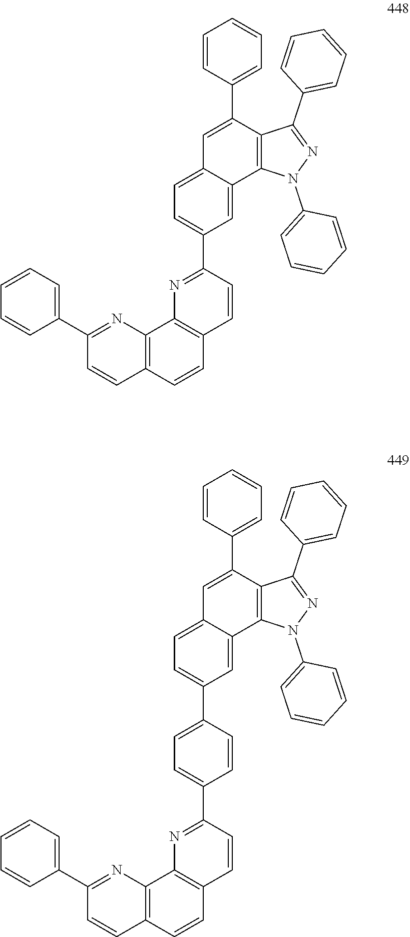 [SMILES: c1ccc(-c2ccc3ccc4ccc(-c5ccc6cc(-c7ccccc7)c7c(-c8ccccc8)nn(-c8ccccc8)c7c6c5)nc4c3n2)cc1 ; c1ccc(-c2ccc3ccc4ccc(-c5ccc(-c6ccc7cc(-c8ccccc8)c8c(-c9ccccc9)nn(-c9ccccc9)c8c7c6)cc5)nc4c3n2)cc1]

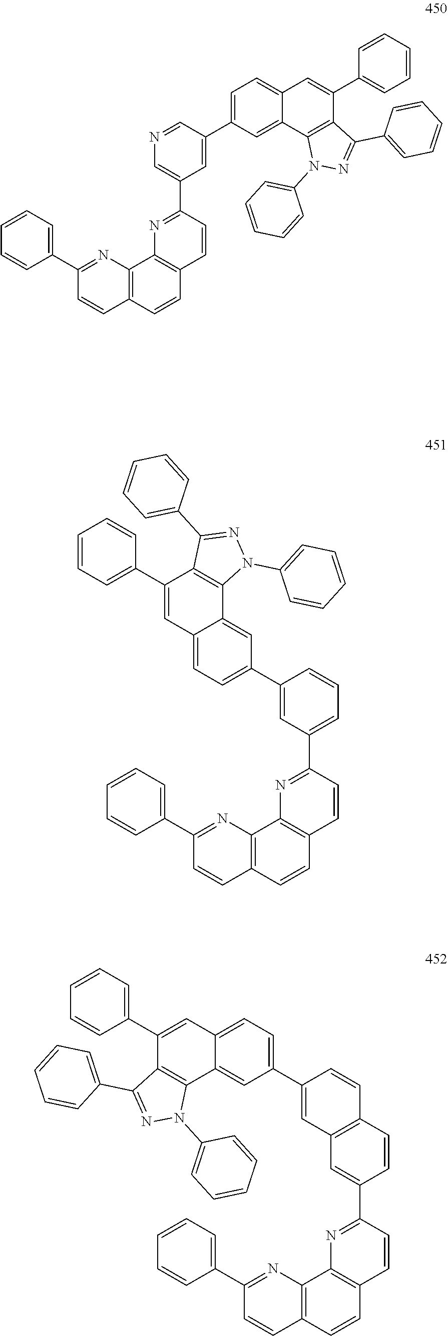 [SMILES: c1ccc(-c2ccc3ccc4ccc(-c5cncc(-c6ccc7cc(-c8ccccc8)c8c(-c9ccccc9)nn(-c9ccccc9)c8c7c6)c5)nc4c3n2)cc1 ; c1ccc(-c2ccc3ccc4ccc(-c5cccc(-c6ccc7cc(-c8ccccc8)c8c(-c9ccccc9)nn(-c9ccccc9)c8c7c6)c5)nc4c3n2)cc1 ; c1ccc(-c2ccc3ccc4ccc(-c5ccc6ccc(-c7ccc8cc(-c9ccccc9)c9c(-c%10ccccc%10)nn(-c%10ccccc%10)c9c8c7)cc6c5)nc4c3n2)cc1]

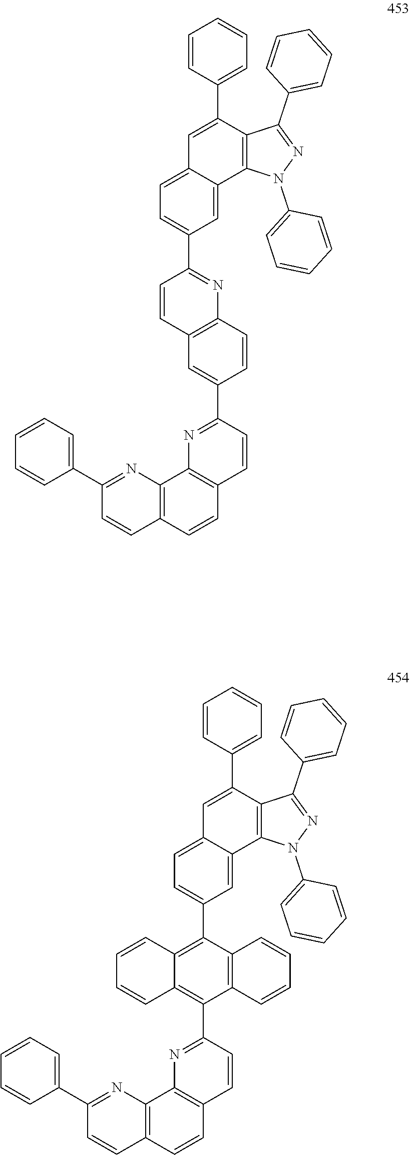 [SMILES: c1ccc(-c2ccc3ccc4ccc(-c5ccc6nc(-c7ccc8cc(-c9ccccc9)c9c(-c%10ccccc%10)nn(-c%10ccccc%10)c9c8c7)ccc6c5)nc4c3n2)cc1 ; c1ccc(-c2ccc3ccc4ccc(-c5c6ccccc6c(-c6ccc7cc(-c8ccccc8)c8c(-c9ccccc9)nn(-c9ccccc9)c8c7c6)c6ccccc56)nc4c3n2)cc1]

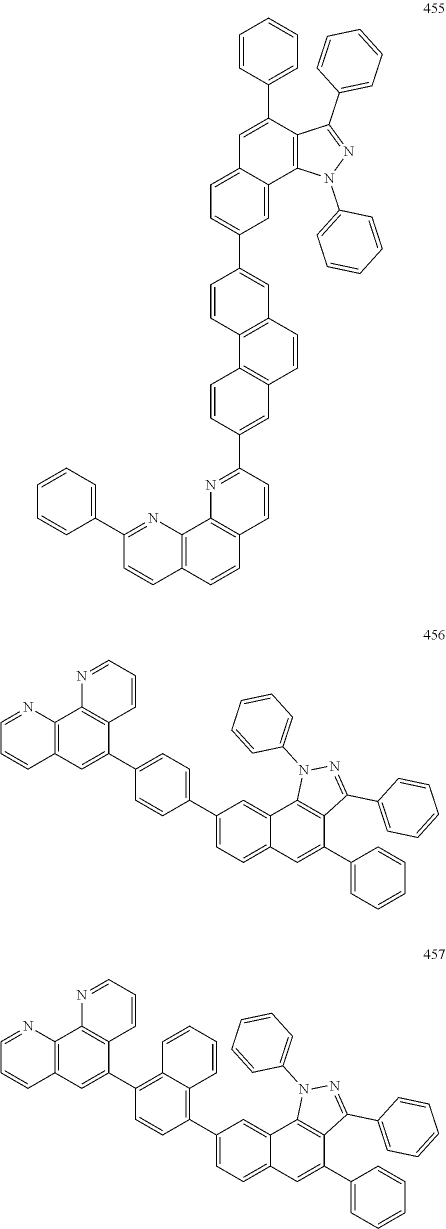 [SMILES: c1ccc(-c2ccc3ccc4ccc(-c5ccc6c(ccc7cc(-c8ccc9cc(-c%10ccccc%10)c%10c(-c%11ccccc%11)nn(-c%11ccccc%11)c%10c9c8)ccc76)c5)nc4c3n2)cc1 ; c1ccc(-c2cc3ccc(-c4ccc(-c5cc6cccnc6c6ncccc56)cc4)cc3c3c2c(-c2ccccc2)nn3-c2ccccc2)cc1 ; c1ccc(-c2cc3ccc(-c4ccc(-c5cc6cccnc6c6ncccc56)c5ccccc45)cc3c3c2c(-c2ccccc2)nn3-c2ccccc2)cc1]

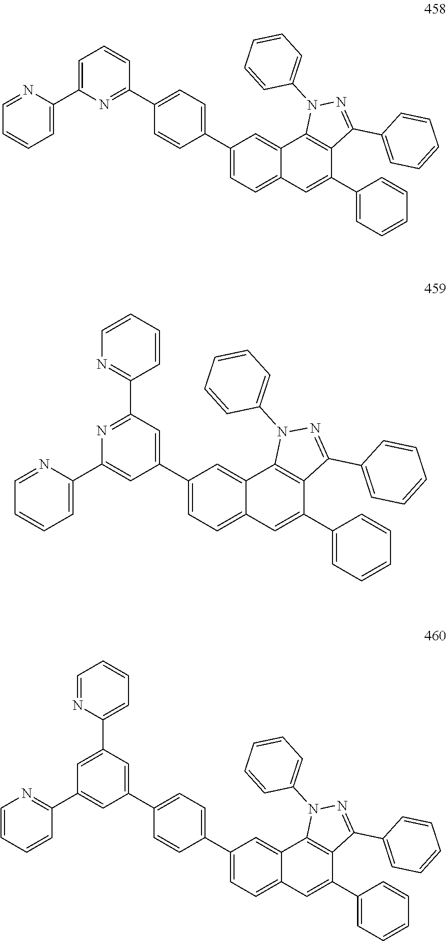 [SMILES: c1ccc(-c2cc3ccc(-c4ccc(-c5cccc(-c6ccccn6)n5)cc4)cc3c3c2c(-c2ccccc2)nn3-c2ccccc2)cc1 ; c1ccc(-c2cc3ccc(-c4cc(-c5ccccn5)nc(-c5ccccn5)c4)cc3c3c2c(-c2ccccc2)nn3-c2ccccc2)cc1 ; c1ccc(-c2cc3ccc(-c4ccc(-c5cc(-c6ccccn6)cc(-c6ccccn6)c5)cc4)cc3c3c2c(-c2ccccc2)nn3-c2ccccc2)cc1]

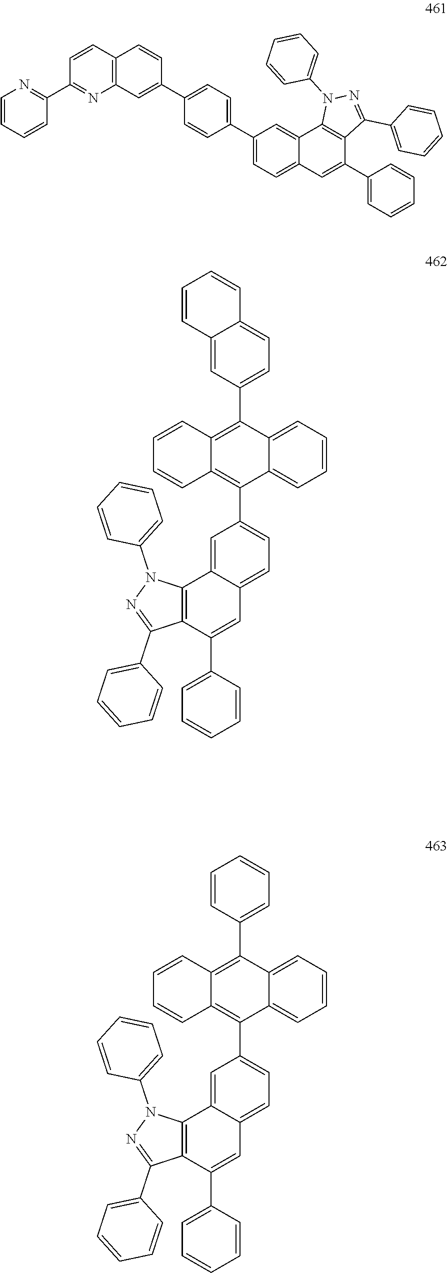 [SMILES: c1ccc(-c2cc3ccc(-c4ccc(-c5ccc6ccc(-c7ccccn7)nc6c5)cc4)cc3c3c2c(-c2ccccc2)nn3-c2ccccc2)cc1 ; c1ccc(-c2cc3ccc(-c4c5ccccc5c(-c5ccc6ccccc6c5)c5ccccc45)cc3c3c2c(-c2ccccc2)nn3-c2ccccc2)cc1 ; c1ccc(-c2c3ccccc3c(-c3ccc4cc(-c5ccccc5)c5c(-c6ccccc6)nn(-c6ccccc6)c5c4c3)c3ccccc23)cc1]

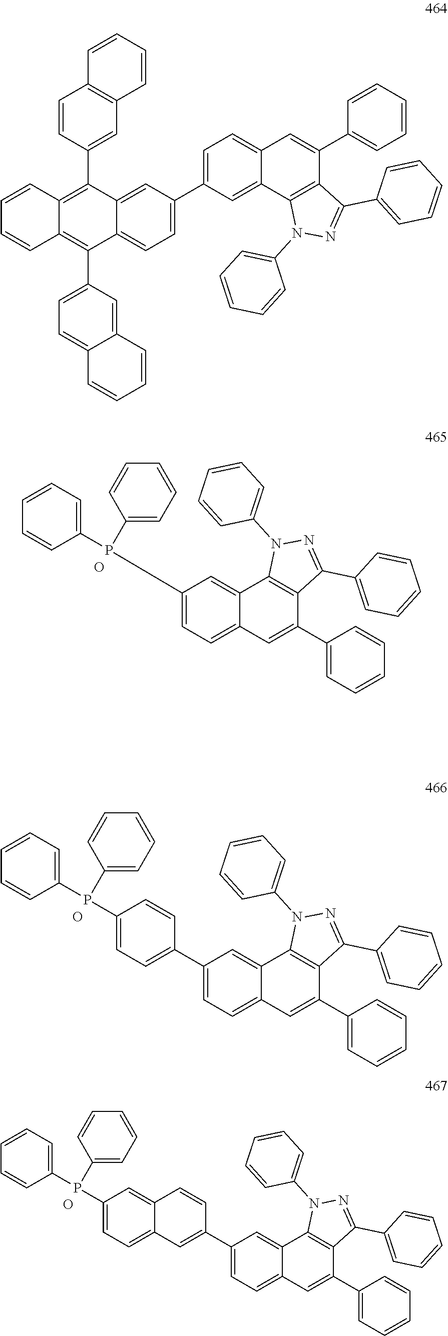

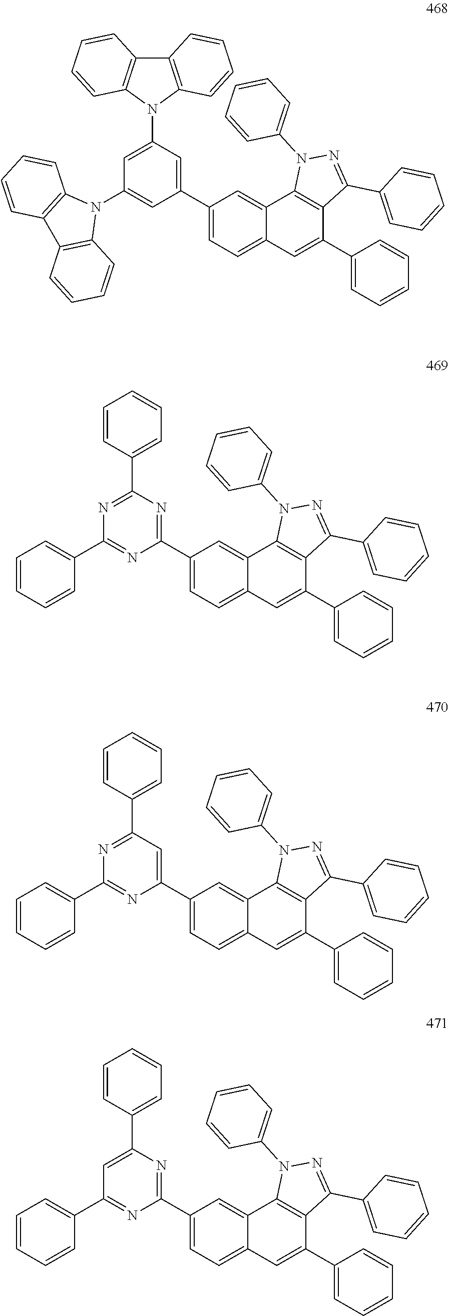 [SMILES: c1ccc(-c2cc3ccc(-c4cc(-n5c6ccccc6c6ccccc65)cc(-n5c6ccccc6c6ccccc65)c4)cc3c3c2c(-c2ccccc2)nn3-c2ccccc2)cc1 ; c1ccc(-c2nc(-c3ccccc3)nc(-c3ccc4cc(-c5ccccc5)c5c(-c6ccccc6)nn(-c6ccccc6)c5c4c3)n2)cc1 ; c1ccc(-c2cc(-c3ccc4cc(-c5ccccc5)c5c(-c6ccccc6)nn(-c6ccccc6)c5c4c3)nc(-c3ccccc3)n2)cc1 ; c1ccc(-c2cc(-c3ccccc3)nc(-c3ccc4cc(-c5ccccc5)c5c(-c6ccccc6)nn(-c6ccccc6)c5c4c3)n2)cc1]

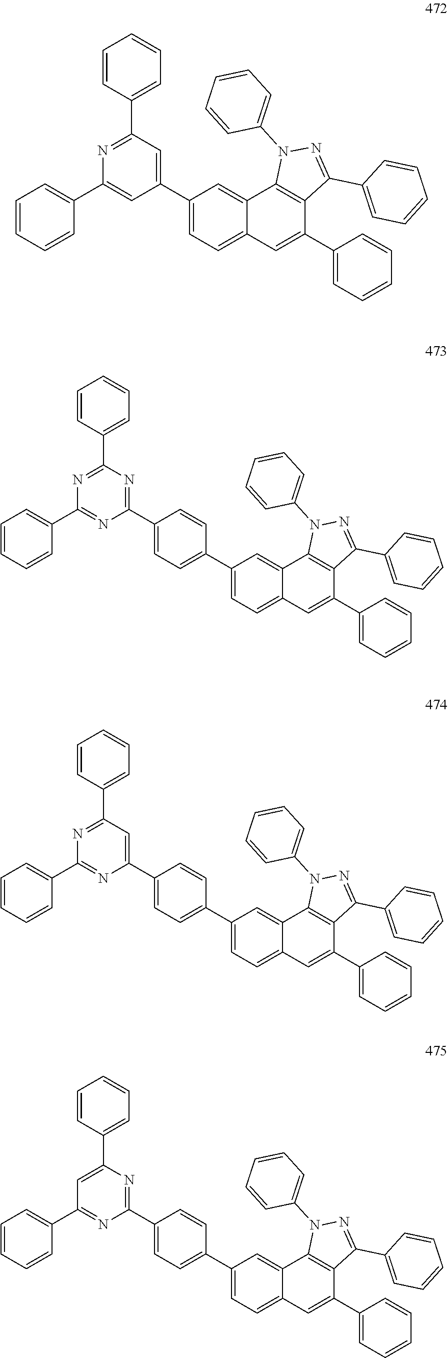 [SMILES: c1ccc(-c2cc(-c3ccc4cc(-c5ccccc5)c5c(-c6ccccc6)nn(-c6ccccc6)c5c4c3)cc(-c3ccccc3)n2)cc1 ; c1ccc(-c2nc(-c3ccccc3)nc(-c3ccc(-c4ccc5cc(-c6ccccc6)c6c(-c7ccccc7)nn(-c7ccccc7)c6c5c4)cc3)n2)cc1 ; c1ccc(-c2cc(-c3ccc(-c4ccc5cc(-c6ccccc6)c6c(-c7ccccc7)nn(-c7ccccc7)c6c5c4)cc3)nc(-c3ccccc3)n2)cc1 ; c1ccc(-c2cc(-c3ccccc3)nc(-c3ccc(-c4ccc5cc(-c6ccccc6)c6c(-c7ccccc7)nn(-c7ccccc7)c6c5c4)cc3)n2)cc1]

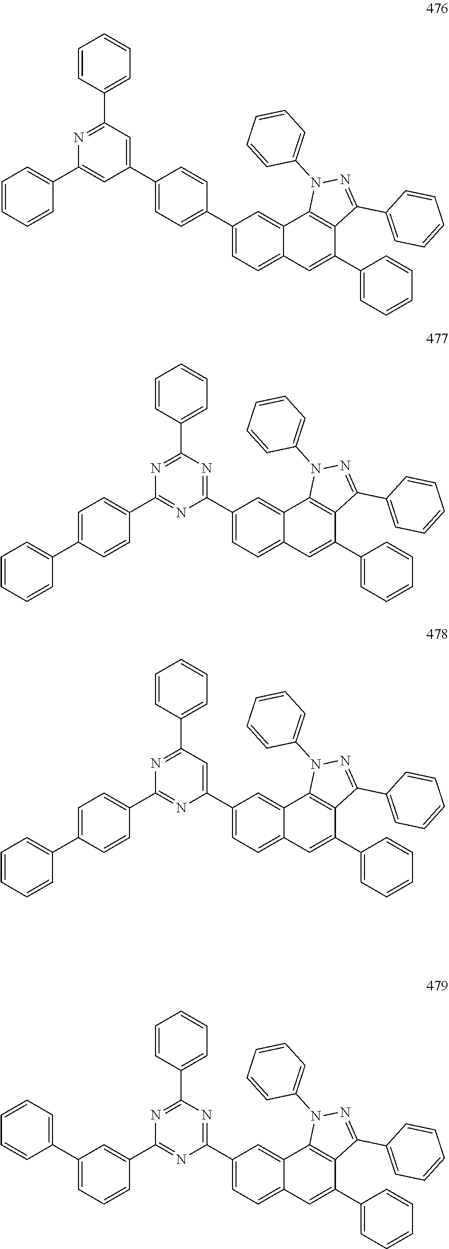 [SMILES: c1ccc(-c2cc(-c3ccc(-c4ccc5cc(-c6ccccc6)c6c(-c7ccccc7)nn(-c7ccccc7)c6c5c4)cc3)cc(-c3ccccc3)n2)cc1 ; c1ccc(-c2ccc(-c3nc(-c4ccccc4)nc(-c4ccc5cc(-c6ccccc6)c6c(-c7ccccc7)nn(-c7ccccc7)c6c5c4)n3)cc2)cc1 ; c1ccc(-c2ccc(-c3nc(-c4ccccc4)cc(-c4ccc5cc(-c6ccccc6)c6c(-c7ccccc7)nn(-c7ccccc7)c6c5c4)n3)cc2)cc1 ; c1ccc(-c2cccc(-c3nc(-c4ccccc4)nc(-c4ccc5cc(-c6ccccc6)c6c(-c7ccccc7)nn(-c7ccccc7)c6c5c4)n3)c2)cc1]

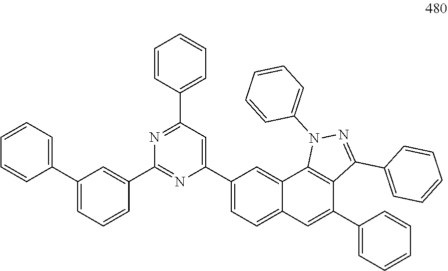 [SMILES: c1ccc(-c2cccc(-c3nc(-c4ccccc4)cc(-c4ccc5cc(-c6ccccc6)c6c(-c7ccccc7)nn(-c7ccccc7)c6c5c4)n3)c2)cc1]

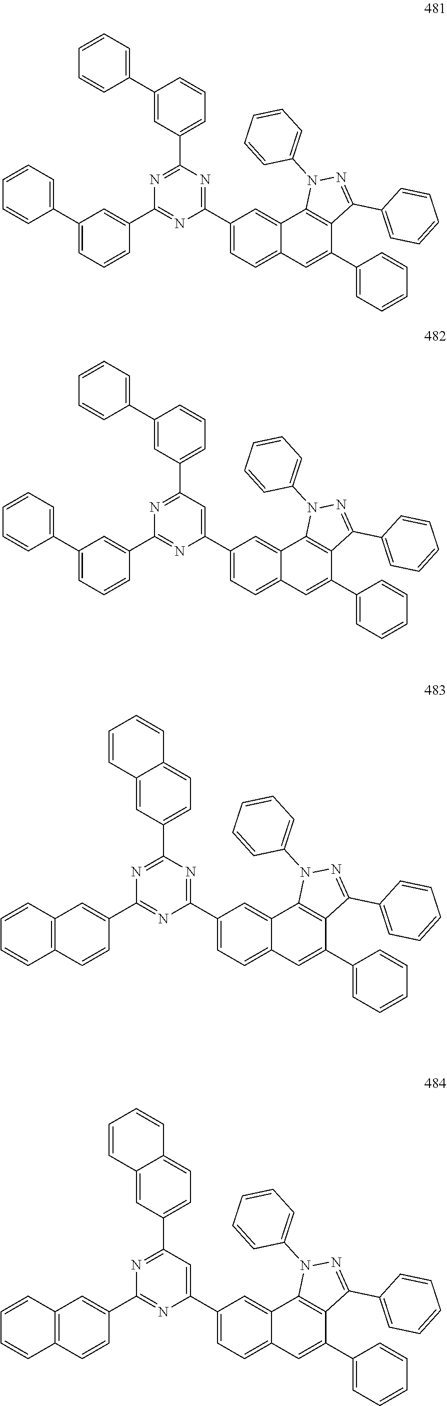

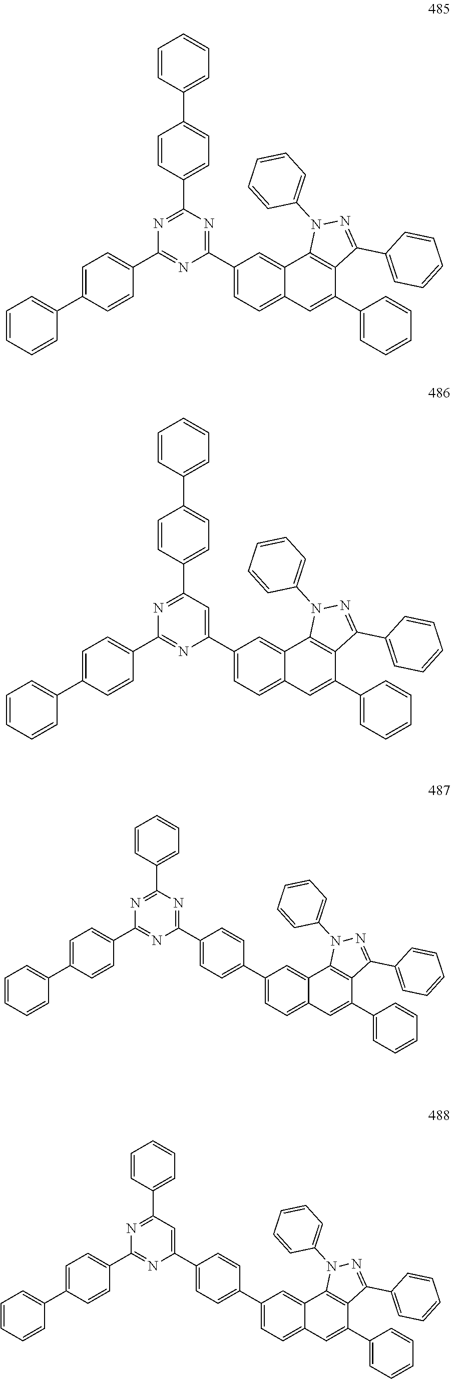

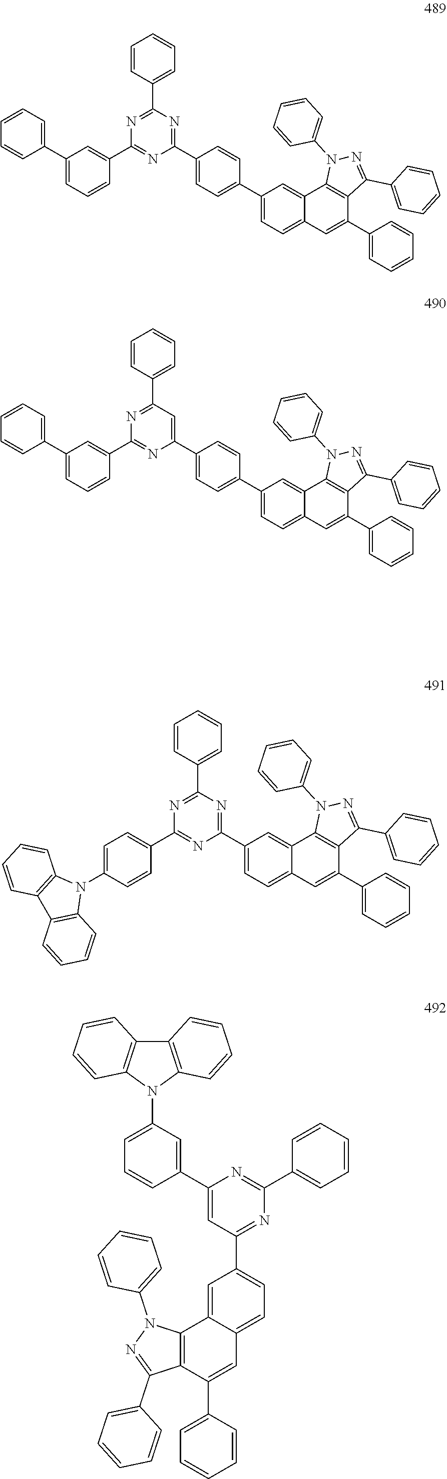

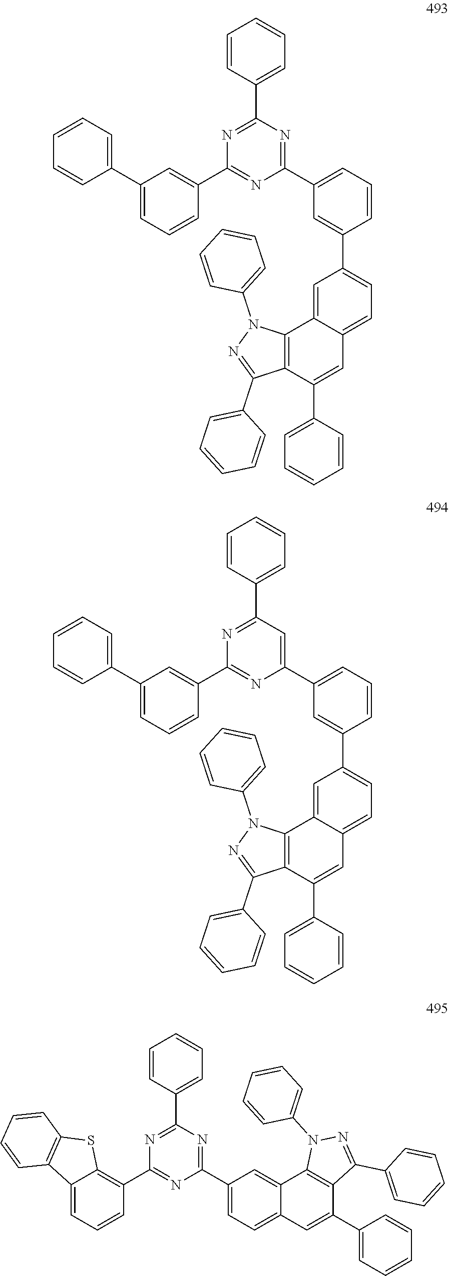

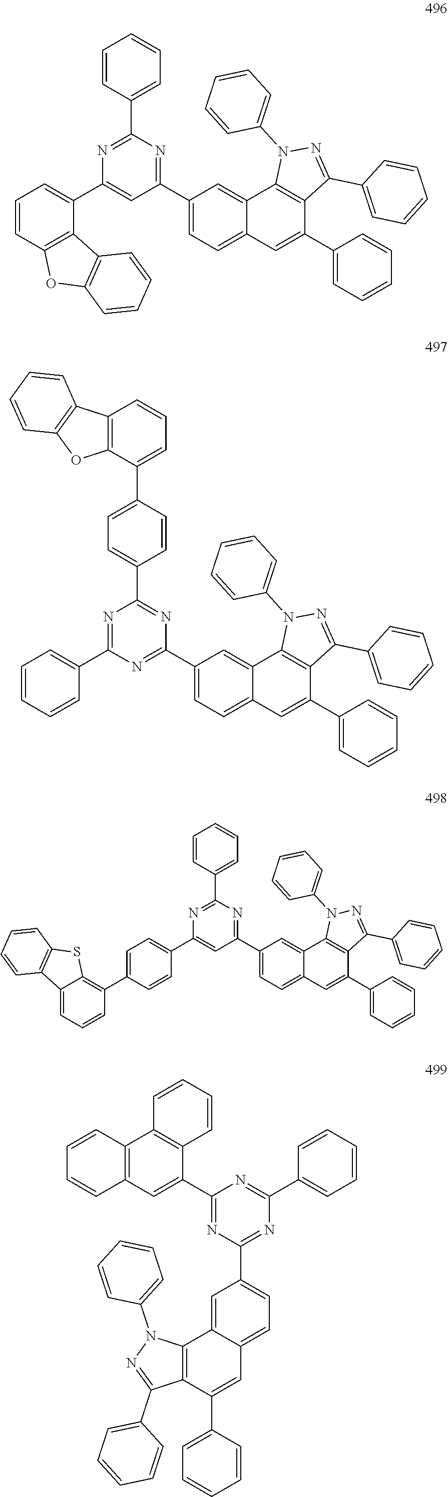

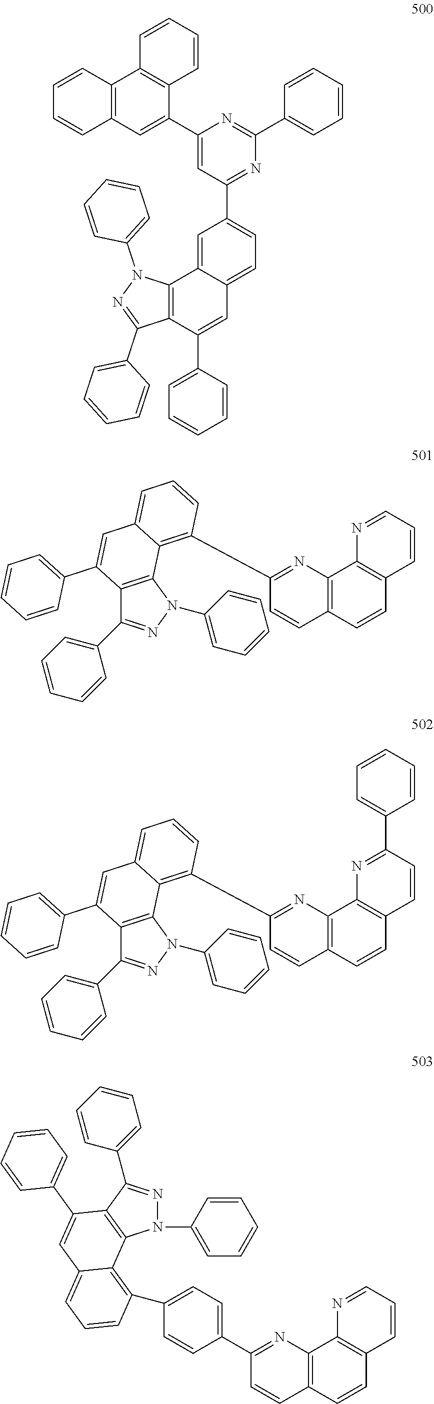

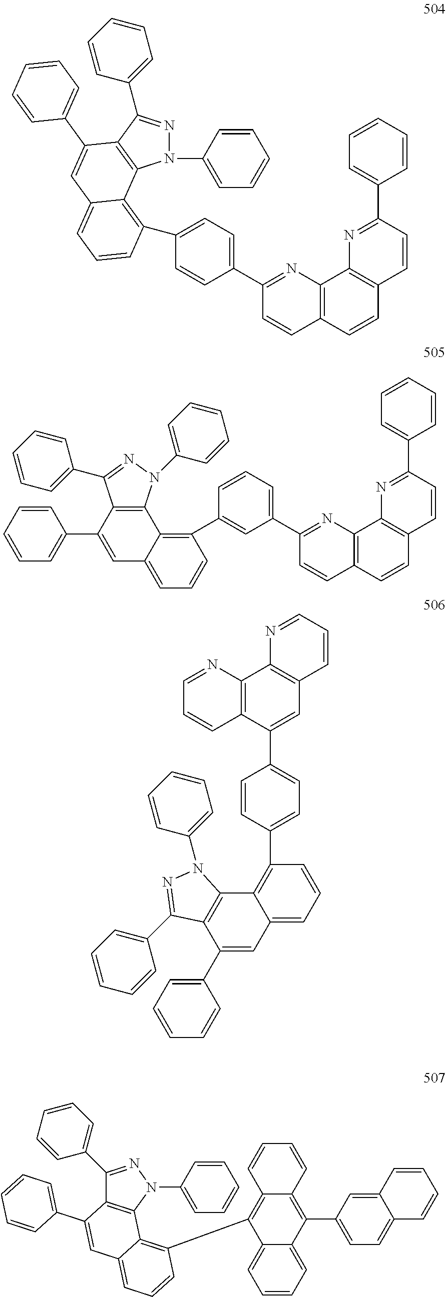

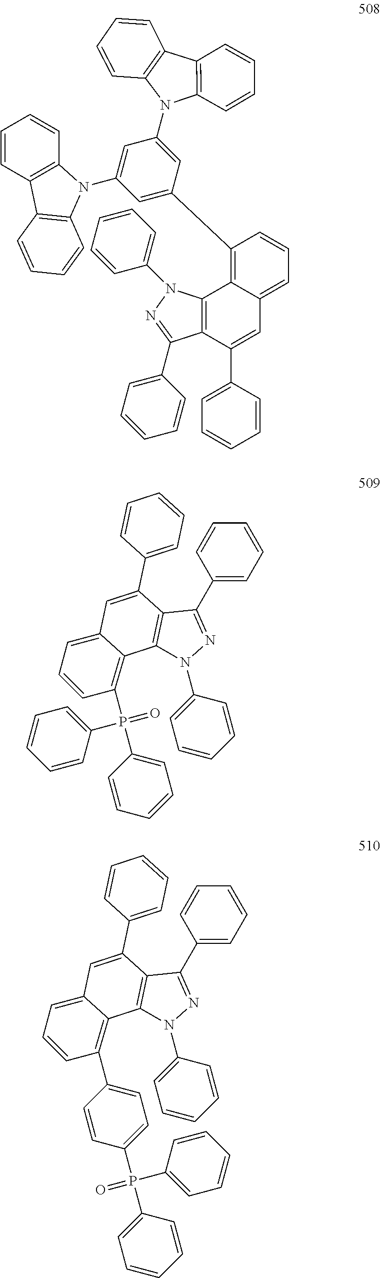

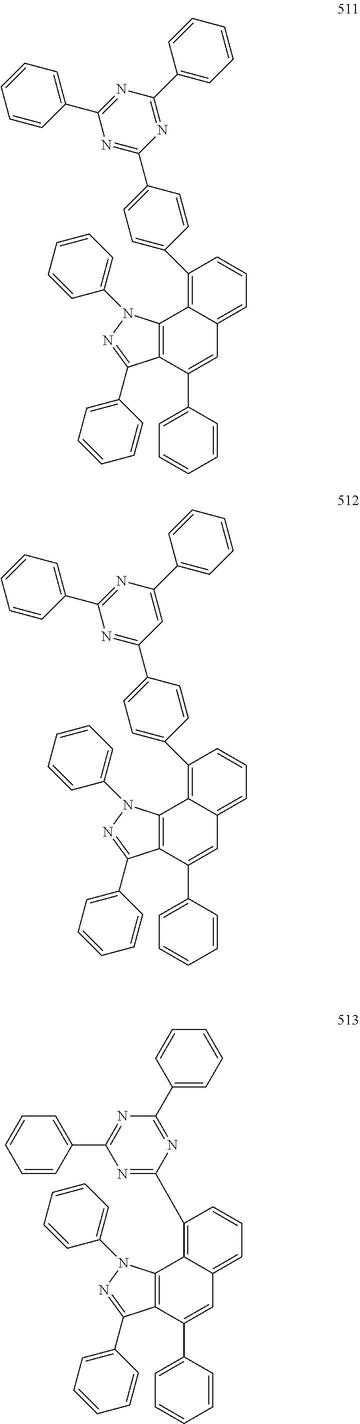

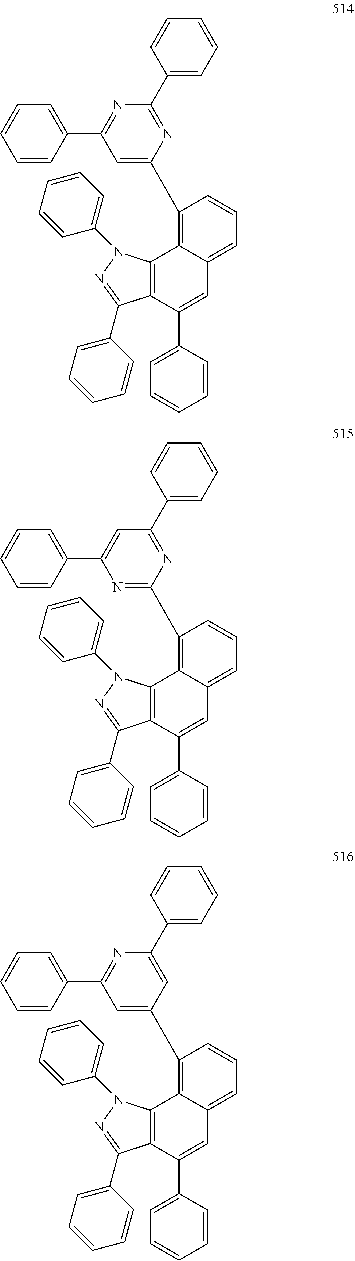

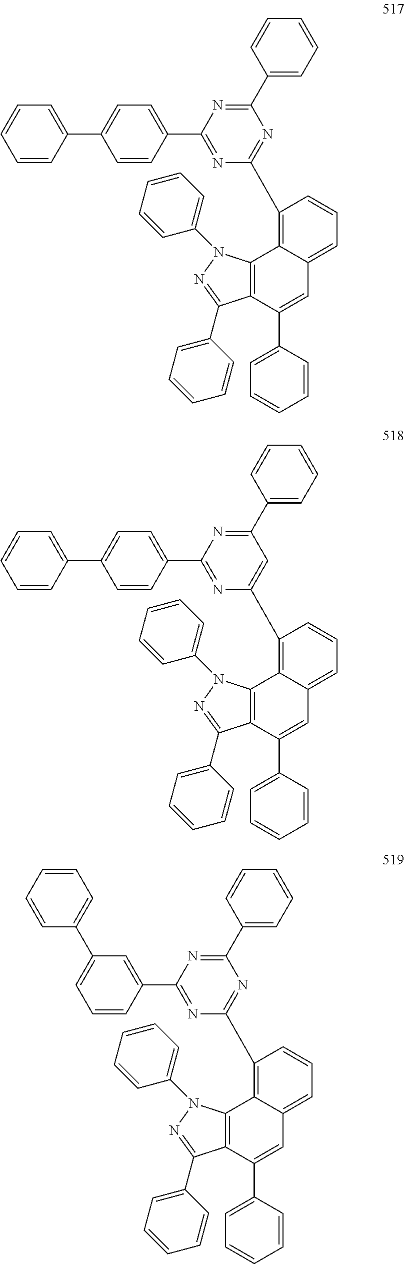

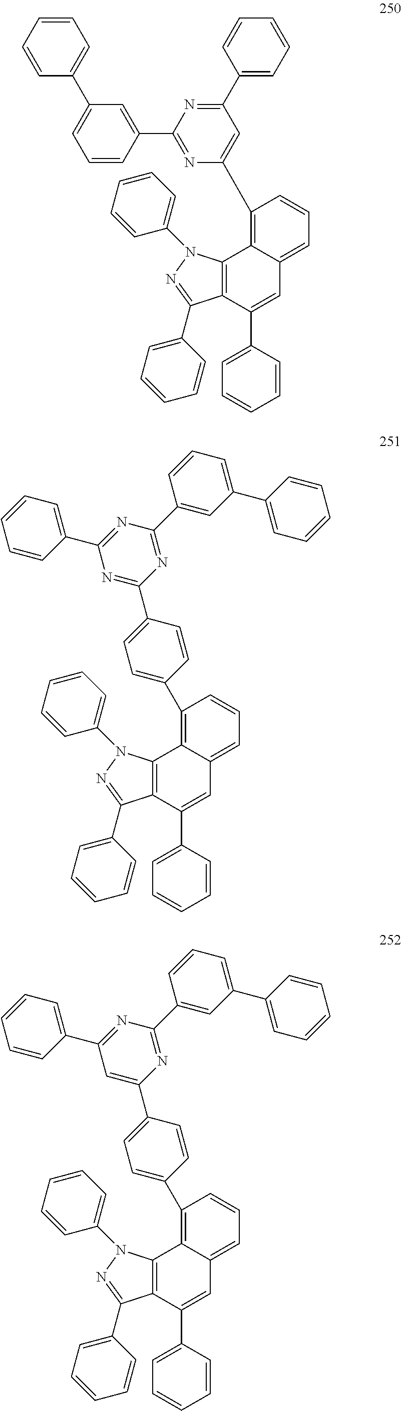

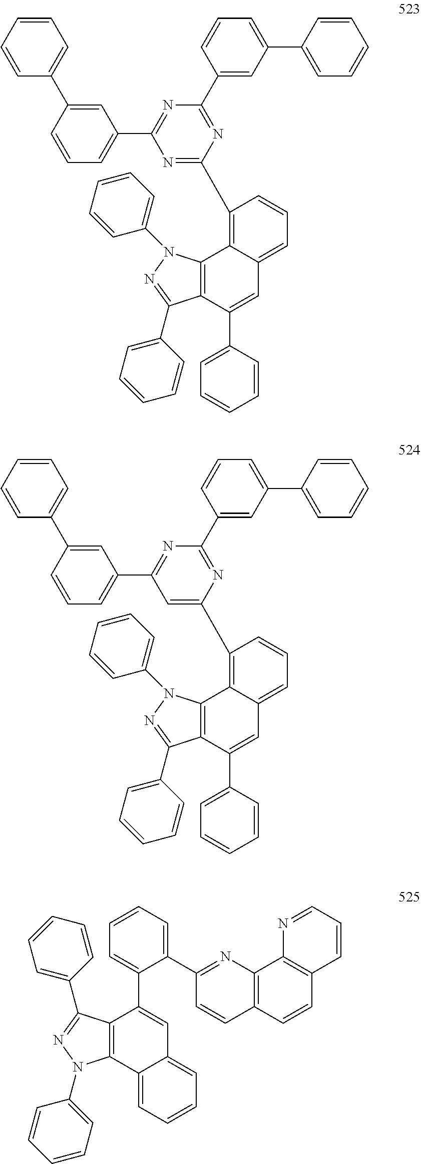 [SMILES: c1ccc(-c2cccc(-c3nc(-c4cccc(-c5ccccc5)c4)nc(-c4cccc5cc(-c6ccccc6)c6c(-c7ccccc7)nn(-c7ccccc7)c6c45)n3)c2)cc1 ; c1ccc(-c2cccc(-c3cc(-c4cccc5cc(-c6ccccc6)c6c(-c7ccccc7)nn(-c7ccccc7)c6c45)nc(-c4cccc(-c5ccccc5)c4)n3)c2)cc1 ; c1ccc(-c2nn(-c3ccccc3)c3c2c(-c2ccccc2-c2ccc4ccc5cccnc5c4n2)cc2ccccc23)cc1]

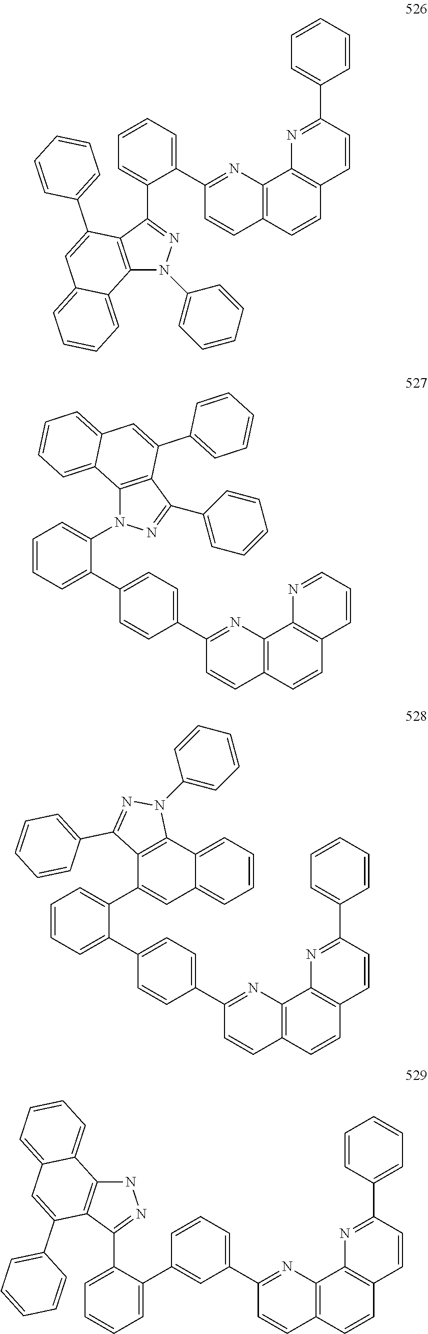

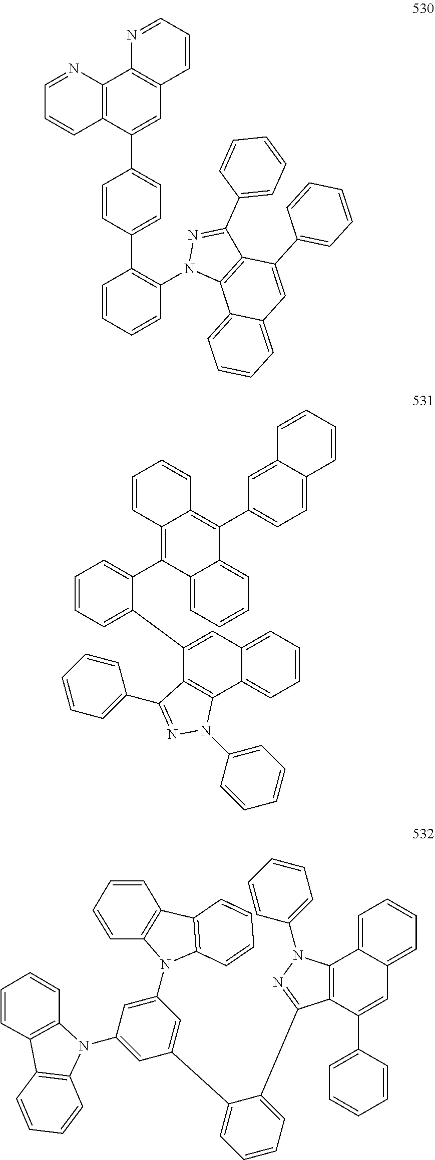 [SMILES: c1ccc(-c2cc3ccccc3c3c2c(-c2ccccc2)nn3-c2ccccc2-c2ccc(-c3cc4cccnc4c4ncccc34)cc2)cc1 ; c1ccc(-c2nn(-c3ccccc3)c3c2c(-c2ccccc2-c2c4ccccc4c(-c4ccc5ccccc5c4)c4ccccc24)cc2ccccc23)cc1 ; c1ccc(-c2cc3ccccc3c3c2c(-c2ccccc2-c2cc(-n4c5ccccc5c5ccccc54)cc(-n4c5ccccc5c5ccccc54)c2)nn3-c2ccccc2)cc1]

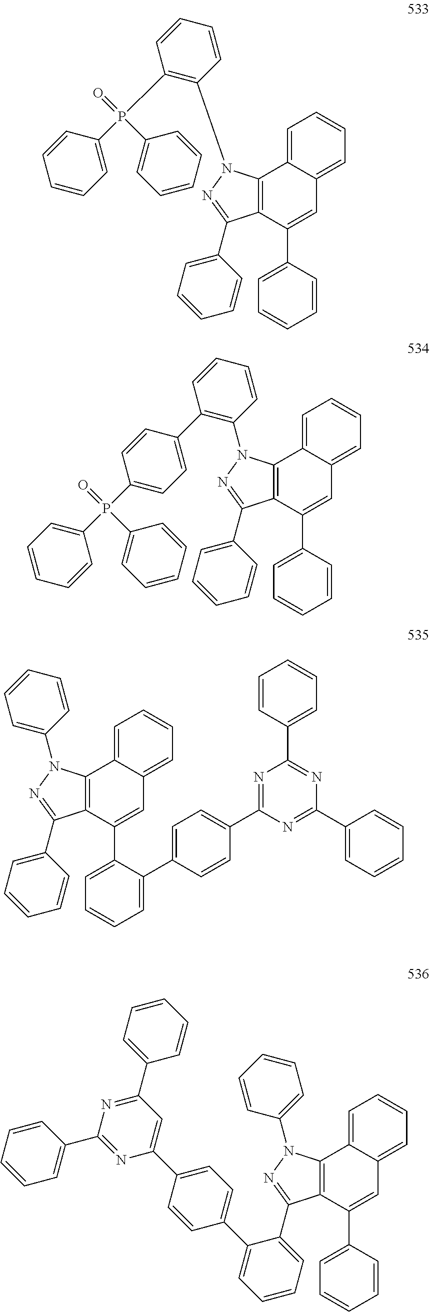

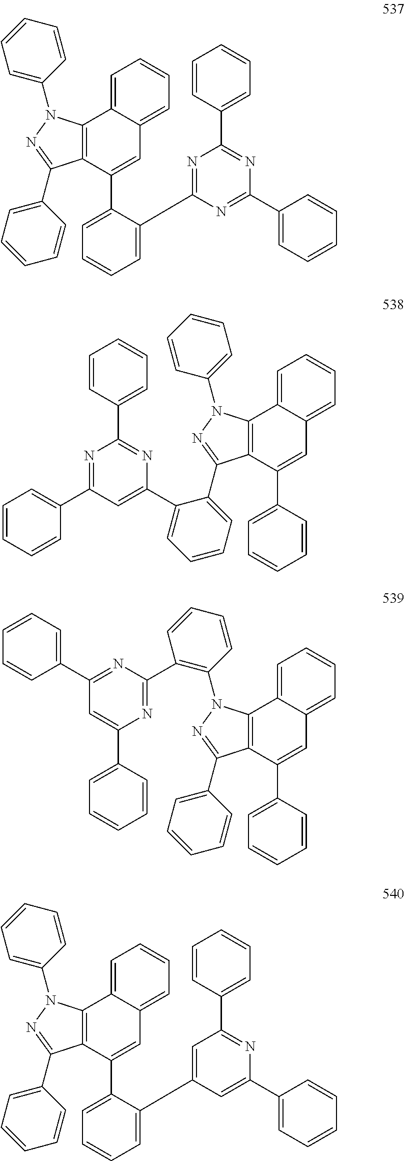

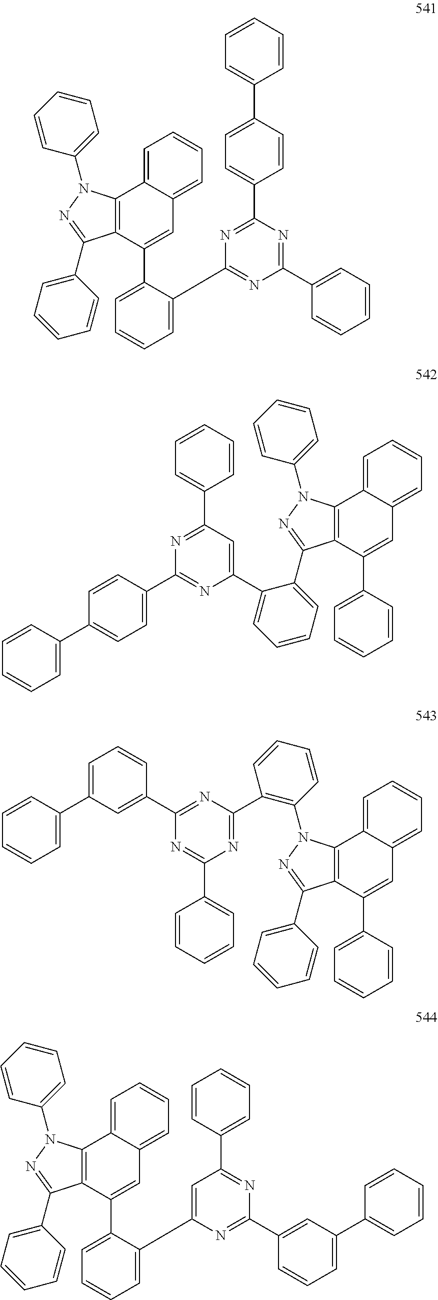 [SMILES: c1ccc(-c2ccc(-c3nc(-c4ccccc4)nc(-c4ccccc4-c4cc5ccccc5c5c4c(-c4ccccc4)nn5-c4ccccc4)n3)cc2)cc1 ; c1ccc(-c2ccc(-c3nc(-c4ccccc4)cc(-c4ccccc4-c4nn(-c5ccccc5)c5c4c(-c4ccccc4)cc4ccccc45)n3)cc2)cc1 ; c1ccc(-c2cccc(-c3nc(-c4ccccc4)nc(-c4ccccc4-n4nc(-c5ccccc5)c5c(-c6ccccc6)cc6ccccc6c54)n3)c2)cc1 ; c1ccc(-c2cccc(-c3nc(-c4ccccc4)cc(-c4ccccc4-c4cc5ccccc5c5c4c(-c4ccccc4)nn5-c4ccccc4)n3)c2)cc1]

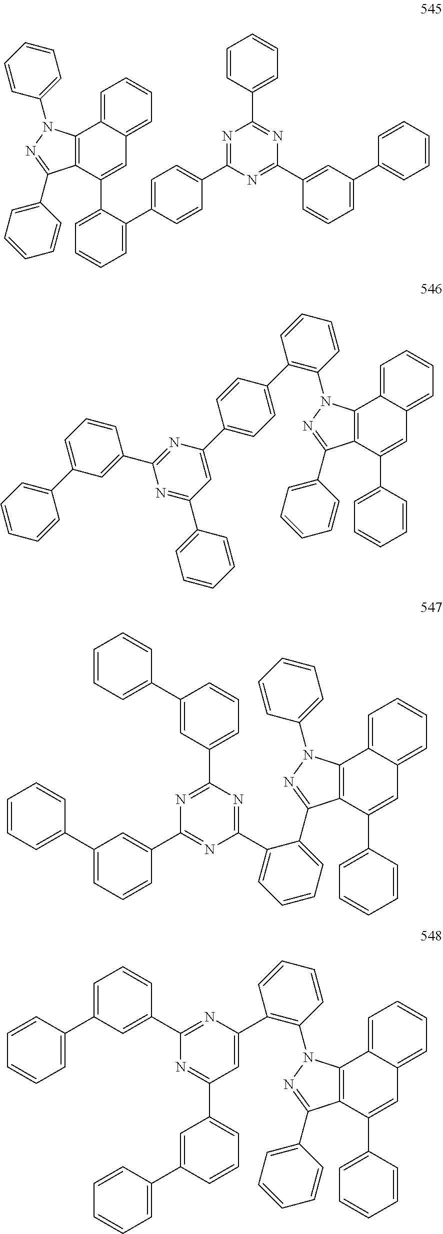 [SMILES: c1ccc(-c2cccc(-c3nc(-c4ccccc4)nc(-c4ccc(-c5ccccc5-c5cc6ccccc6c6c5c(-c5ccccc5)nn6-c5ccccc5)cc4)n3)c2)cc1 ; c1ccc(-c2cccc(-c3nc(-c4ccccc4)cc(-c4ccc(-c5ccccc5-n5nc(-c6ccccc6)c6c(-c7ccccc7)cc7ccccc7c65)cc4)n3)c2)cc1 ; c1ccc(-c2cccc(-c3nc(-c4cccc(-c5ccccc5)c4)nc(-c4ccccc4-c4nn(-c5ccccc5)c5c4c(-c4ccccc4)cc4ccccc45)n3)c2)cc1 ; c1ccc(-c2cccc(-c3cc(-c4ccccc4-n4nc(-c5ccccc5)c5c(-c6ccccc6)cc6ccccc6c54)nc(-c4cccc(-c5ccccc5)c4)n3)c2)cc1]

The compound of Formula 1 may be synthesized as a compound having intrinsic properties of the introduced substituent by introducing various substituents into the corresponding structure. For example, by introducing a substituent mainly used for a hole injection layer material, a hole transport layer material, an electron-blocking layer material, a light-emitting layer material, a hole-blocking layer material, an electron transport layer material, an electron injection layer material, and an electron-generating layer material used in manufacturing the organic light-emitting device into the core structure, it is possible to synthesize a material satisfying the conditions required for each organic layer.

In addition, by introducing various substituents into the structure of the compound of Formula 1, it is possible to finely control the energy band gap, while by improving the properties at the interface between organic materials, it is possible to diversify the use of the material.

The heterocyclic compound may be used as one or more uses selected from a hole injection layer material, a hole transport layer material, an electron-blocking layer material, a light-emitting layer material, a hole-blocking layer material, an electron transport layer material, an electron injection layer material, and an electron-generating layer material used in the organic layer of the organic light-emitting device, substantially, may be used as an electron transport layer material, a charge-generating layer material, an electron injection layer material, an electron-blocking layer material, and a hole-blocking layer material, and in particular, may be preferably used as an electron transport layer material and a charge-generating layer material.

In addition, the present invention relates to an light-emitting device comprising:
a first electrode; a second electrode provided to face the first electrode; and one or more organic layers provided between the first electrode and the second electrode, and
wherein one or more of the organic layers comprise the heterocyclic compound represented by Formula 1.

In one embodiment of the present invention, the first electrode may be an anode, and the second electrode may be a cathode.

In another embodiment, the first electrode may be a cathode, and the second electrode may be an anode.

The organic light-emitting device according to one embodiment of the present invention may further comprise one or more layers selected from the group consisting of a hole injection layer, a hole transport layer, an electron-blocking layer, a light-emitting layer, a hole-blocking layer, an electron transport layer, and an electron injection layer, and they may have, but are not limited to, a stack structure in the order of anode/hole injection layer/hole transport layer/electron-blocking layer/light-emitting layer/hole-blocking layer/electron transport layer/electron injection layer/cathode.

In one embodiment of the present invention, the organic light-emitting device may be a green organic light-emitting device, and the heterocyclic compound represented by Formula 1 may be used as a material of the green organic light-emitting device.

In one embodiment of the present invention, the organic light-emitting device may be a blue organic light-emitting device, and the heterocyclic compound represented by Formula 1 may be used as a material of the blue organic light-emitting device.

In one embodiment of the present invention, the organic light-emitting device may be a red organic light-emitting device, and the heterocyclic compound represented by Formula 1 may be used as a material of the red organic light-emitting device.

In one embodiment of the present invention, the organic light-emitting device may be a white organic light-emitting device, and the heterocyclic compound represented by Formula 1 may be used as a material of the white organic light-emitting device.

Specific contents of the heterocyclic compound represented by Formula 1 are the same as described above.

The organic light-emitting device of the present invention may be manufactured by conventional methods and materials for manufacturing an organic light-emitting device, except that one or more organic layers are formed using the aforementioned heterocyclic compound.

In one embodiment of the present invention, the heterocyclic compound represented by Formula 1 may be used as one or more uses selected from a hole injection layer material, a hole transport layer material, an electron-blocking layer material, a light-emitting layer material, a hole-blocking layer material, an electron transport layer material, an electron injection layer material, and a charge-generating layer material in the green organic light-emitting device, the blue organic light-emitting device, the red organic light-emitting device, and the white organic light-emitting device, substantially, may be used as an electron transport layer material, a charge-generating layer material, an electron injection layer material, an electron-blocking layer material, and a hole-blocking layer material, and in particular, may be preferably used as an electron transport layer material and a charge-generating layer material.

The accompanying FIGS. 1 to 3 illustrate the stack order of the electrodes and the organic layers of the organic light-emitting device according to one embodiment of the present invention. However, it is not intended that the scope of the present invention be limited by these drawings, and the structure of an organic light-emitting device known in the art may also be applied to the present invention.

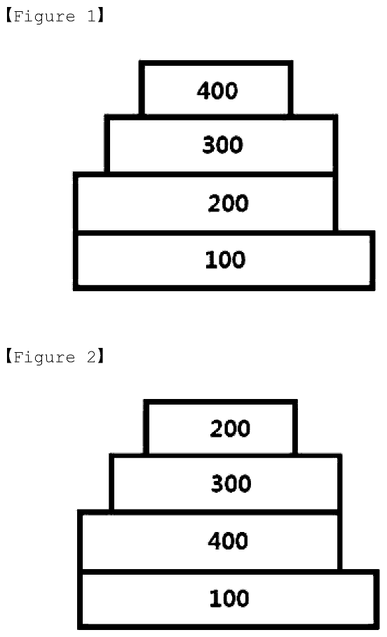

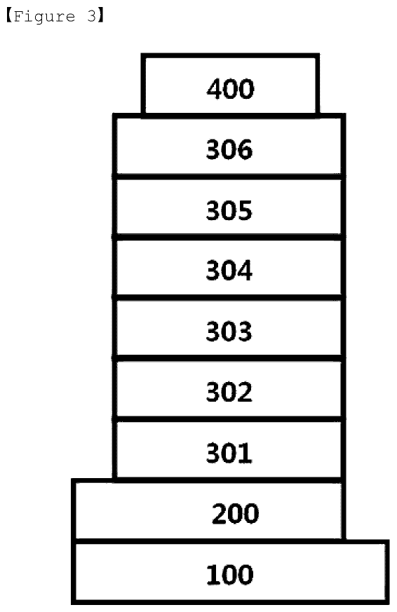

Referring to FIG. 1, an organic light-emitting device in which an anode 200, an organic layer 300, and a cathode 400 are sequentially stacked on a substrate 100 is illustrated. However, it is not limited to such a structure, and an organic light-emitting device in which a cathode, an organic layer, and an anode are sequentially stacked on a substrate may be implemented, as shown in FIG. 2.

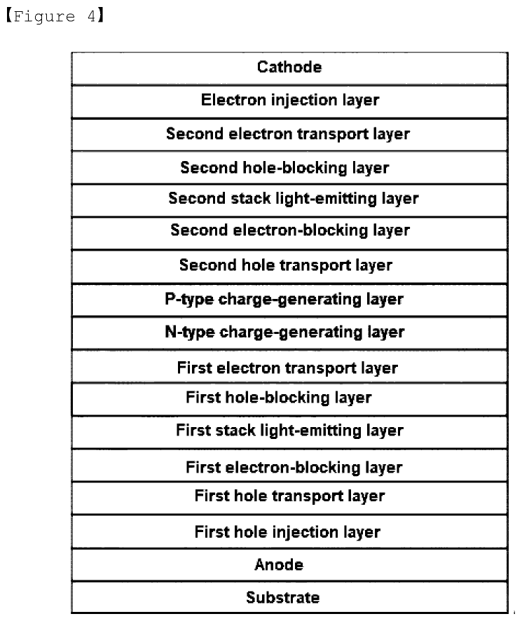

FIG. 3 illustrates a case where the organic layer is multi-layered. The organic light-emitting device according to FIG. 3 comprises a hole injection layer 301, a hole transport layer 302, a light-emitting layer 303, a hole-blocking layer 304, an electron transport layer 305, and an electron injection layer 306. However, the scope of the present invention is not limited by such stack structures, and the remaining layers except for the light-emitting layer may be omitted, if necessary, and other necessary functional layers such as an electron-blocking layer may be further added.

According to one embodiment of the present invention, the organic light-emitting device may have a tandem structure in which two or more independent devices are connected in series. In one embodiment, the tandem structure may have a form in which each organic light-emitting device is bonded to a charge-generating layer. Since a device having a tandem structure may be driven at a lower current than a unit device based on the same brightness, there is an advantage in that the lifespan property of the device are greatly improved.

According to one embodiment of the present invention, the organic layer comprises a first stack including one or more light-emitting layers; a second stack including one or more light-emitting layers; and one or more charge-generating layers provided between the first stack and the second stack.

According to another embodiment of the present invention, the organic layer comprises a first stack including one or more light-emitting layers; a second stack including one or more light-emitting layers; and a third stack including one or more light-emitting layers, and comprises one or more charge-generating layers, respectively, between the first stack and the second stack and between the second stack and the third stack.

The charge-generating layer may mean a layer in which holes and electrons are generated when a voltage is applied. The charge-generating layer may be an N-type charge-generating layer or a P-type charge-generating layer. In the present invention, the N-type charge-generating layer means a charge-generating layer located closer to the anode than the P-type charge-generating layer, and the P-type charge-generating layer means a charge-generating layer located closer to the cathode than the N-type charge-generating layer.

The N-type charge-generating layer and the P-type charge-generating layer may be provided in contact with each other, and in this case, an NP junction is formed. By the NP junction, holes are easily formed in the P-type charge-generating layer and electrons are easily formed in the N-type charge-generating layer. Electrons are transported in the anode direction through the LUMO level of the N-type charge-generating layer, and holes are transported in the cathode direction through the HOMO level of the P-type charge-generating layer.

The first stack, the second stack, and the third stack each independently include one or more light-emitting layers, and may further include one or more layers of a hole injection layer, a hole transport layer, an electron-blocking layer, a layer that transports and injects holes at the same time (hole injection and transport layer), and a layer that transports and injects electrons at the same time (electron injection and transport layer).

An organic light-emitting diode including the first stack and the second stack is illustrated in FIG. 4. However, it is not intended that the scope of the present invention be limited by these drawings, and the structure of an organic light-emitting device known in the art may also be applied to the present invention.

The first electron-blocking layer, the first hole-blocking layer, the second hole-blocking layer, and the like described in FIG. 4 may be omitted in some cases.

According to one embodiment of the present invention, the charge-generating layer including the heterocyclic compound of Formula 1 may be an N-type charge-generating layer, and the charge-generating layer may further include dopants known in the art other than the heterocyclic compound of Formula 1.

The organic light-emitting device of the present invention may be manufactured by conventional methods and materials for manufacturing an organic light-emitting device, except that one or more organic layers are formed using the aforementioned heterocyclic compound.

The heterocyclic compound may form an organic layer by a solution coating method as well as a vacuum deposition method when manufacturing an organic light-emitting device. Wherein the solution coating method refers to, but is not limited to, spin coating, dip coating, inkjet printing, screen printing, spraying, roll coating, and the like.

The organic layer of the organic light-emitting device of the present invention may have a single-layer structure, but may have a multi-layer structure in which two or more organic layers are stacked. For example, the organic light-emitting device of the present invention may have a structure comprising one or more selected from the group consisting of a hole injection layer, a hole transport layer, an electron-blocking layer, light-emitting layer, a hole-blocking layer, an electron transport layer, an electron injection layer, an electron-generating layer, and the like, as an organic layer. However, the structure of the organic light-emitting device is not limited to such a structure, and may include a smaller or larger number of organic layers.

In the organic light-emitting device according to one embodiment of the present invention, materials other than the heterocyclic compound represented by Formula 1 are exemplified below, but these are for illustration only and not for limiting the scope of the present invention, and may be replaced with materials known in the art.

Materials having a relatively large work function may be used as the anode material, and transparent conductive oxides, metals, conductive polymers, or the like may be used. Specific examples of the anode material include, but are not limited to, metals such as vanadium, chromium, copper, zinc, and gold, or alloys thereof; metal oxides such as zinc oxide, indium oxide, indium tin oxide (ITO), and indium zinc oxide (IZO); a combination of metals and oxides such as ZnO: Al or SnO₂: Sb; conductive polymers such as poly(3-methylthiophene), poly[3,4-(ethylene-1,2-dioxy)thiophene] (PEDOT), polypyrrole, and polyaniline; and the like.

Materials having a relatively low work function may be used as the cathode material, and metals, metal oxides, conductive polymers, or the like may be used. Specific examples of the cathode material include, but are not limited to, metals such as magnesium, calcium, sodium, potassium, titanium, indium, yttrium, lithium, gadolinium, aluminum, silver, tin, and lead, or alloys thereof; multilayer structure materials such as LiF/Al or LiO₂/Al.

As the hole injection layer material, a known hole injection layer material may be used, for example, phthalocyanine compounds such as copper phthalocyanine, and the like, disclosed in U.S. Pat. No. 4,356,429, or starburst-type amine derivatives such as tris(4-carbazolyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), soluble conductive polymer polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, disclosed in Advanced Material, 6, p.677 (1994) may be used.

As the hole transport layer material, a pyrazoline derivative, an arylamine-based derivative, a stilbene derivative, a triphenyldiamine derivative, and the like may be used, and a low molecular weight or high molecular weight material may be used.

As the electron transport layer material, oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone and derivatives thereof, diphenyldicyanoethylene and derivatives thereof, diphenoquinone derivatives, metal complexes of 8-hydroxyquinoline and derivatives thereof, and the like may be used, and high molecular weight materials as well as low molecular weight materials may be used.

As the electron injection layer material, for example, LiF is typically used in the art, but the present invention is not limited thereto.

As the light-emitting layer material, a red, green, or blue light-emitting material may be used, and a mixture of two or more light-emitting materials may be used, if necessary. In this case, it is possible to use by depositing two or more light-emitting materials as separate sources, or it is possible to use by premixing and depositing them as a single source. In addition, as the light-emitting layer material, a fluorescent material may be used, or a phosphorescent material may be used. As the light-emitting layer material, materials that emit light by combining holes and electrons respectively injected from the anode and the cathode may be used alone, or materials in which the host material and the dopant material together participate in light emission may be used.

When using by mixing hosts of the light-emitting layer material, it is possible to use by mixing hosts of the same series, or it is possible to use by mixing hosts of different series. For example, it is possible to use by selecting any two or more types of n-type host material and p-type host material as the host material of the light-emitting layer.

In the phosphorescent material, those known in the art may be used as the phosphorescent dopant material. For example, phosphorescent dopant materials represented by LL′MX′, LL′L″M, LMX′X″, L₂MX′, and L₃M may be used, but the scope of the present invention is not limited by these examples.
Wherein M may be iridium, platinum, osmium, or the like.
Wherein L is an anionic bidentate ligand coordinated to M by sp² carbon and a heteroatom, and X may function to trap electrons or holes. Non-limiting examples of L include 2-(1-naphthyl)benzoxazole, (2-phenylbenzoxazole), (2-phenylbenzothiazole), (7,8-benzoquinoline), (thiophenepyrizine), phenylpyridine, benzothiophenepyrizine, 3-methoxy-2-phenylpyridine, thiophenepyrizine, tolylpyridine, and the like. Non-limiting examples of X′ and X″ include acetylacetonate (acac), hexafluoroacetylacetonate, salicylidene, picolinate, 8-hydroxyquinolinate, and the like.

Specific examples of the phosphorescent dopant are shown below, but are not limited to these examples.

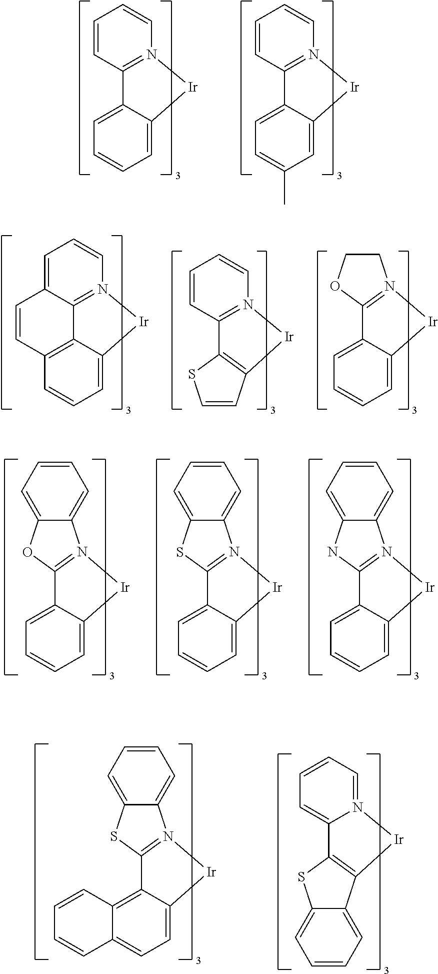

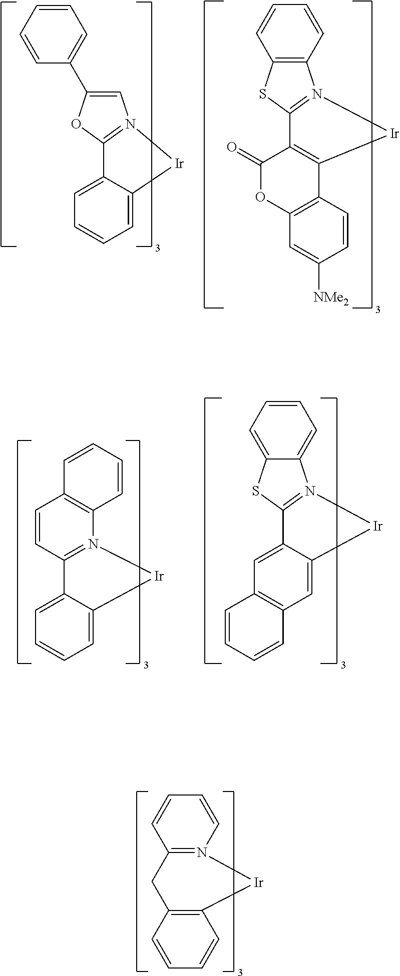

In one embodiment of the present invention, the light-emitting layer includes the heterocyclic compound represented by Formula 1, and may be used together with an iridium-based dopant.

In one embodiment of the present invention, as the iridium-based dopant, the red phosphorescent dopant (piq) 2 (Ir) (acac), the green phosphorescent dopant Ir(ppy) 3, and the like may be used.

In one embodiment of the present invention, the content of the dopant may have a content of 1% to 15%, preferably 3% to 10%, more preferably 5% to 10% based on the entire light-emitting layer.

The electronic-blocking layer material may include, but is limited to, one or more compounds selected from tris(phenyloyrazole)iridium, 9, 9-bis[4—(N,N-bis-biphenyl-4-ylamino)phenyl]-9H-fluorene (BPAPF), bis[4-(p,p-ditolylamino)phenyl]diphenylsilane, NPD (4,4′-bis[N-(1-napthyl)-N-phenylamino]biphenyl), mCP (N,N′-dicarbazolyl-3,5-benzene), and MPMP (bis[4—(N,N-diethylamino)-2-methylphenyl] (4-methylphenyl)methane).

In addition, the electron-blocking layer may include an inorganic compound. For example, it may include, but is not limited to, at least any one or a combination of halide compounds such as LiF, NaF, KF, RbF, CsF, FrF, MgF₂, CaF₂, SrF₂, BaF₂, LiCl, NaCl, KCl, RbCl, CsCl, FrCl, and the like; and oxides such as Li₂O, Li₂O₂, Na₂O, K₂O, Rb₂O, Rb₂O₂, Cs₂O, Cs₂O₂, LiAlO₂, LiBO₂, LiTaO₃, LiNbO₃, LiWO₄, Li₂CO, NaWO₄, KAlO₂, K₂SiO₃, B₂O₅, Al₂O₃, SiO₂, and the like.

The hole-blocking layer material may include, but is not limited to, an oxadiazole derivative, a triazole derivative, a phenanthroline derivative, BCP, an aluminum complex, and the like. The N-type charge-generating layer may include, but is not limited to, 2,3,5,6-tetrafluoro-7,7,8,8-tetracyanoquinodimethane (F4TCNQ), fluorine-substituted 3,4,9,10-perylenetetracarboxylic dianhydride (PTCDA), cyano-substituted PTCDA, naphthalenetetracarboxylic dianhydride (NTCDA), fluorine-substituted NTCDA, cyano-substituted NTCDA, hexaazatriphenylline derivatives and the like. In one embodiment, the N-type charge-generating layer may include a benzimidazophenanthrine-based derivative and a Li metal at the same time.

The P-type charge-generating layer may include an arylamine-based derivative and a cyano group-containing compound at the same time.

In the organic light-emitting device of the present invention, materials known in the art may be used without limitation as materials not described above.

The organic light-emitting device according to one embodiment of the present invention may be a top emission type, a bottom emission type, or a dual emission type depending on the material to be used.

In addition, the present invention relates to a composition for an organic layer of a organic light-emitting device comprising the heterocyclic compound represented by Formula 1.

Specific contents of the heterocyclic compound represented by Formula 1 are the same as described above.

The composition for an organic layer may be used as a hole injection layer material, a hole transport layer material, an electron-blocking layer material, a light-emitting layer material, a hole-blocking layer material, an electron transport layer material, an electron injection layer material, and a charge-generating layer material, substantially, may be used as an electron transport layer material, a charge-generating layer material, an electron injection layer material, an electron-blocking layer material, and a hole-blocking layer material, and in particular, may be preferably used as an electron transport layer material and a charge-generating layer material.

If it is used as the charge-generating layer material, it may be used as the N-type charge-generating layer material.

The composition for an organic layer may further include materials commonly used in the composition for an organic layer in the art, together with the heterocyclic compound represented by Formula 1.

In addition, the present invention relates to a method of manufacturing a organic light-emitting device, comprising the steps of:
preparing a substrate; forming a first electrode on the substrate; forming one or more organic layers on the first electrode; and forming a second electrode on the organic layer, wherein the step of forming the organic layers comprises the step of forming one or more organic layers using the heterocyclic compound represented by Formula 1 or the composition for an organic layer of the present invention.

In one embodiment of the present invention, the step of forming the organic layers may form the organic layers using the heterocyclic compound represented by Formula 1 or the composition for an organic material layer through a thermal vacuum deposition method.

The organic layer including the composition for an organic layer may further include other materials commonly used in the art, if necessary.

The heterocyclic compound represented by Formula 1 according to one embodiment of the present invention may act on a principle similar to that applied to the organic light-emitting device even in an organic electronic device including an organic solar cell, an organic photoreceptor, an organic transistor, and the like.

Hereinafter, preferred examples will be provided to help to understand the present invention, but the following examples are provided not to limit the present invention but to facilitate the understanding of the present invention.

### Preparative Examples

### [Preparative Example 1] Preparation of Compound 1

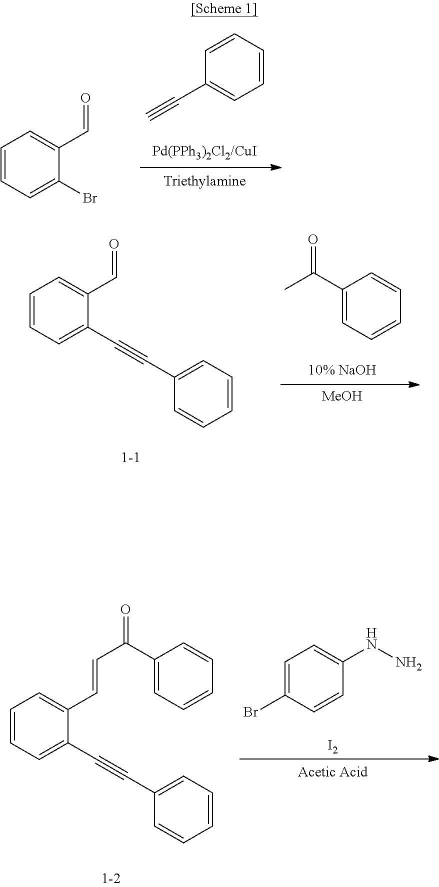

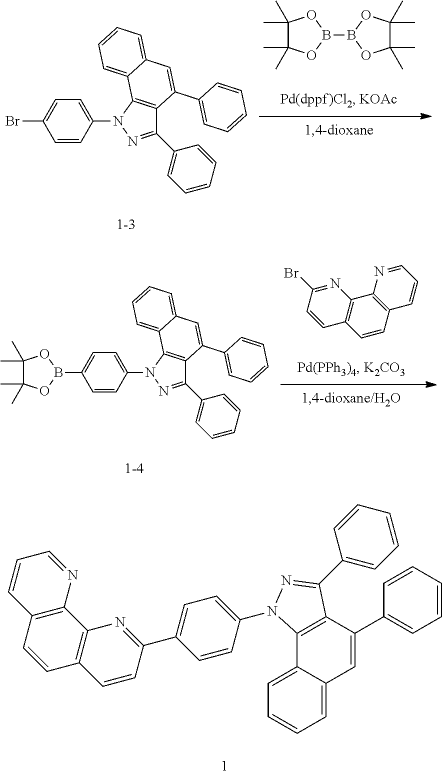

### 1) Preparation of Compound 1-1

2-bromobenzaldehyde (60 g, 324.29 mmol, 1 eq), ethynylbenzene (36.4 g, 356.72 mmol, 1.1 eq), bis(triphenylphosphine)palladium(II) dichloride (4.56 g, 6.49 mmol, 0.02 eq), and copper iodide (0.61 g, 3.24 mmol, 0.01 eq) were placed in 600 ml of trimethylamine and stirred at 60° C. for 6 hours. The solution was passed through Celite and then washed with MC. The solvent was concentrated and then passed through silica gel. The solvent was removed to give 58 g of Compound 1-1 in a yield of 87%.

### 2) Preparation of Compound 1-2

Compound 1-1 (58 g, 281.23 mmol, 1 eq) and acetophenone (37.2 g, 309.35 mmol, 1.1 eq) were placed in 10% aqueous sodium hydroxide solution (58 ml) and methanol (580 ml) and stirred at room temperature for 3 hours. After completion of the reaction, the precipitated solid was filtered and washed with water and methanol. 72 g of compound 1-2 was obtained in a yield of 83%.

### 3) Preparation of Compound 1-3

Compound 1-2 (72 g, 233.48 mmol, 1 eq) was added to acetic acid (720 ml), and then (4-bromophenyl)hydrazine (52.4 g, 280.18 mmol, 1.2 eq) and iodine (71.1 g, 280.18) mmol, 1.2 eq) were placed therein with stirring and stirred under reflux for 8 hours. After completion of the reaction, the reaction solution was cooled to room temperature and diluted with distilled water, and then neutralized with an aqueous sodium hydrogen carbonate solution. It was extracted with ethyl acetate and distilled water, and then separated by a silica gel column (developing solvent MC:Hex=1:5) to give 100 g of compound 1-3 in a yield of 75%.

### 3) Preparation of Compound 1-4

Compound 1-3 (100 g, 210.35 mmol, 1 eq), bis(pinacolato)diboron (80.1 g, 315.52 mmol, 1.5 eq), PdCl₂(dppf) (15.39 g, 21.04 mmol, 0.1 eq), and KOAc (41.29 g, 420.70 mmol, 2 eq) were placed in 1000 ml of 1,4-dioxane and stirred at 100° C. for 6 hours. It was extracted with MC and water, and then the organic layer was dried over anhydrous Na₂SO₄ and filtered through silica gel. After it was precipitated with MC/MeOH, the precipitate was filtered to give 91 g of Compound 1-4 in a yield of 83%.

4) Preparation of Compound 1

Compound 1-4 (11.4 g, 21.86 mmol, 1 eq) and 2-bromo-1,10-phenanthroline (5.66 g, 21.86 mmol, 1 eq) were dissolved in 110 ml of 1,4-dioxane and 25 ml of distilled water, and then Pd(PPh₃)₄ (1.26 g, 1.09 mmol, 0.05 eq) and K₂CO₃ (6.04 g, 43.72 mmol, 2 eq) were placed therein and stirred under reflux for 15 hours. MC was placed and dissolved in the reaction solution, and then extracted with water, and the organic layer was dried over anhydrous Na₂SO₄. It was passed through a silica gel filter and then precipitated with MC/MeOH. The precipitated solid was filtered to give 10.8 g of Compound 1 in a yield of 88%.

Target compound A in Table 1 below was synthesized in the same manner as in Preparative Example 1, except that Intermediate A in Table 1 below was used instead of 2-bromo-1,10-phenanthroline.

TABLE 1

| Com- | | | |
|---|---|---|---|
| pound | | | |
| No. | Intermediate A | Target Compound A | Yield |
| 8 | 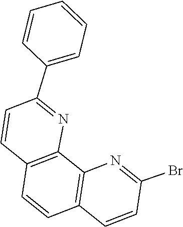 [SMILES: Brc1ccc2ccc3ccc(-c4ccccc4)nc3c2n1] | 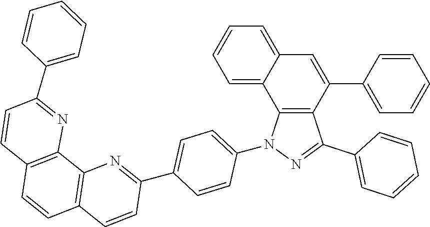 [SMILES: c1ccc(-c2ccc3ccc4ccc(-c5ccc(-n6nc(-c7ccccc7)c7c(-c8ccccc8)cc8ccccc8c76)cc5)nc4c3n2)cc1] | 78% |
| 9 | 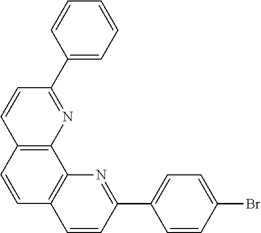 [SMILES: Brc1ccc(-c2ccc3ccc4ccc(-c5ccccc5)nc4c3n2)cc1] | 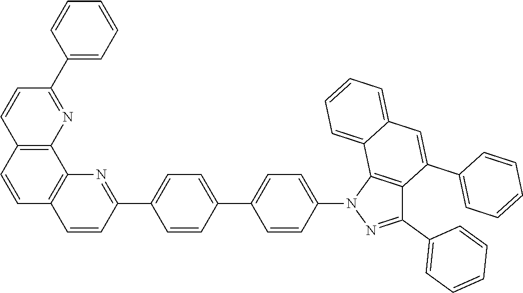 [SMILES: c1ccc(-c2ccc3ccc4ccc(-c5ccc(-c6ccc(-n7nc(-c8ccccc8)c8c(-c9ccccc9)cc9ccccc9c87)cc6)cc5)nc4c3n2)cc1] | 82% |
| 26 | 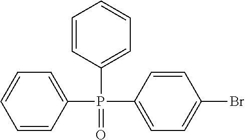 [SMILES: O=P(c1ccccc1)(c1ccccc1)c1ccc(Br)cc1] | 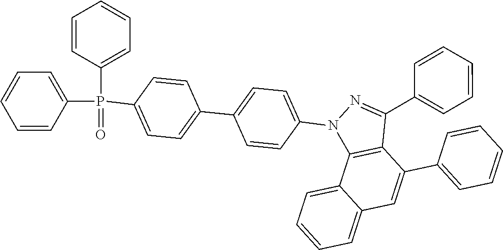 [SMILES: O=P(c1ccccc1)(c1ccccc1)c1ccc(-c2ccc(-n3nc(-c4ccccc4)c4c(-c5ccccc5)cc5ccccc5c43)cc2)cc1] | 84% |
| 29 | 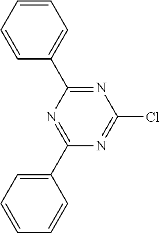 [SMILES: Clc1nc(-c2ccccc2)nc(-c2ccccc2)n1] | 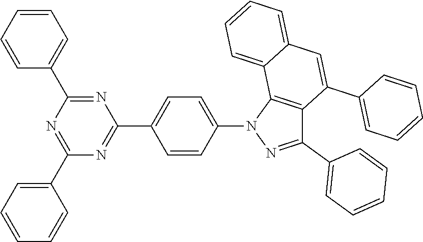 [SMILES: c1ccc(-c2nc(-c3ccccc3)nc(-c3ccc(-n4nc(-c5ccccc5)c5c(-c6ccccc6)cc6ccccc6c54)cc3)n2)cc1] | 79% |
| 30 | 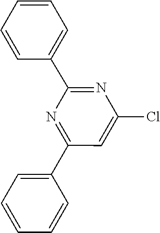 [SMILES: Clc1cc(-c2ccccc2)nc(-c2ccccc2)n1] | 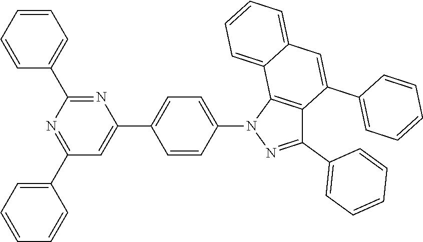 [SMILES: c1ccc(-c2cc(-c3ccc(-n4nc(-c5ccccc5)c5c(-c6ccccc6)cc6ccccc6c54)cc3)nc(-c3ccccc3)n2)cc1] | 68% |
| 37 | 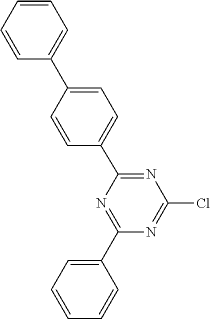 [SMILES: Clc1nc(-c2ccccc2)nc(-c2ccc(-c3ccccc3)cc2)n1] | 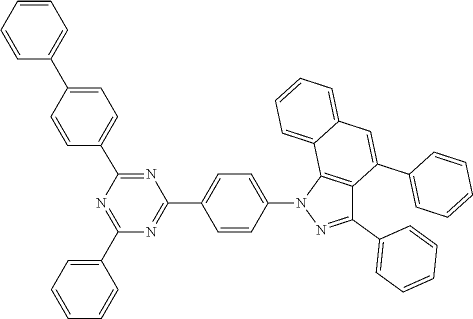 [SMILES: c1ccc(-c2ccc(-c3nc(-c4ccccc4)nc(-c4ccc(-n5nc(-c6ccccc6)c6c(-c7ccccc7)cc7ccccc7c65)cc4)n3)cc2)cc1] | 71% |
| 38 | 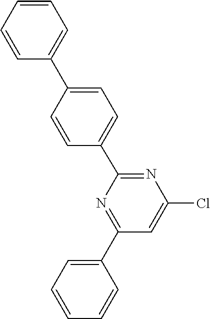 [SMILES: Clc1cc(-c2ccccc2)nc(-c2ccc(-c3ccccc3)cc2)n1] | 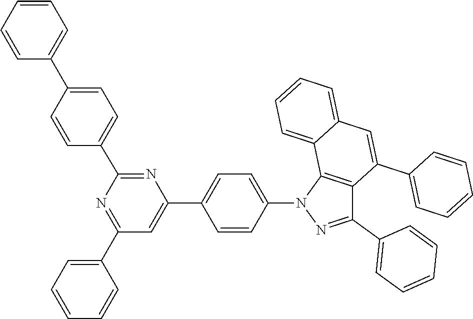 [SMILES: c1ccc(-c2ccc(-c3nc(-c4ccccc4)cc(-c4ccc(-n5nc(-c6ccccc6)c6c(-c7ccccc7)cc7ccccc7c65)cc4)n3)cc2)cc1] | 69% |
| 39 | 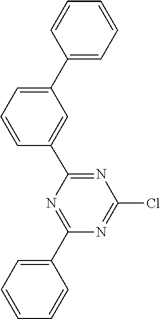 [SMILES: Clc1nc(-c2ccccc2)nc(-c2cccc(-c3ccccc3)c2)n1] | 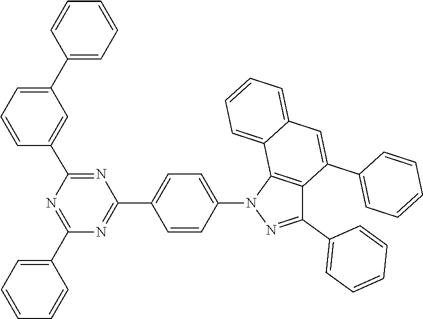 [SMILES: c1ccc(-c2cccc(-c3nc(-c4ccccc4)nc(-c4ccc(-n5nc(-c6ccccc6)c6c(-c7ccccc7)cc7ccccc7c65)cc4)n3)c2)cc1] | 83% |
| 46 | 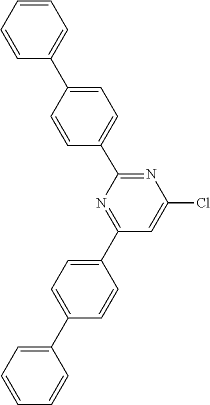 [SMILES: Clc1cc(-c2ccc(-c3ccccc3)cc2)nc(-c2ccc(-c3ccccc3)cc2)n1] | 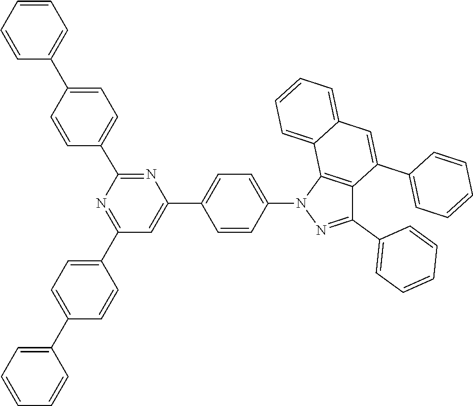 [SMILES: c1ccc(-c2ccc(-c3cc(-c4ccc(-n5nc(-c6ccccc6)c6c(-c7ccccc7)cc7ccccc7c65)cc4)nc(-c4ccc(-c5ccccc5)cc4)n3)cc2)cc1] | 86% |

### [Preparative Example 2] Preparation of Compound 61

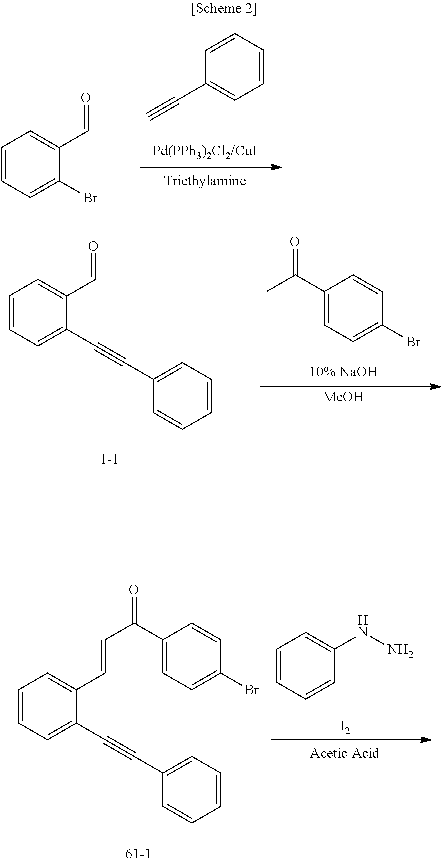

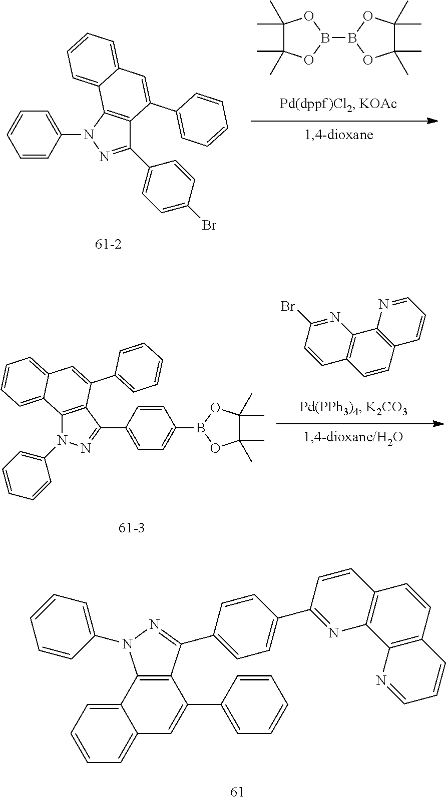

### 1) Preparation of Compound 61-1

Compound 1-1 (58 g, 281.23 mmol, 1 eq) and 1-(4-bromophenyl)ethan-1-one (61.6 g, 309.35 mmol, 1.1 eq) were placed in 10% aqueous sodium hydroxide solution (58 ml) and methanol (580 ml) and stirred at room temperature for 5 hours. After completion of the reaction, the precipitated solid was filtered and washed with water and methanol. 90.4 g of compound 61-1 was obtained in a yield of 83%.

### 2) Preparation of Compound 61-2

Compound 61-1 (72 g, 233.48 mmol, 1 eq) was added to acetic acid (720 ml), and then phenylhydrazine (30.3 g, 280.18 mmol, 1.2 eq) and iodine (71.1 g, 280.18) mmol, 1.2 eq) were placed therein with stirring and stirred under reflux for 7 hours. After completion of the reaction, the reaction solution was cooled to room temperature and diluted with distilled water, and then neutralized with an aqueous sodium hydrogen carbonate solution. It was extracted with ethyl acetate and distilled water, and then separated by a silica gel column (developing solvent MC:Hex=1:5) to give 100 g of compound 61-2 in a yield of 75%.

### 3) Preparation of Compound 61-3

Compound 61-2 (100 g, 210.35 mmol, 1 eq), bis(pinacolato)diboron (80.1 g, 315.52 mmol, 1.5 eq), PdCl₂(dppf) (15.39 g, 21.04 mmol, 0.1 eq), and KOAc (41.29 g, 420.70 mmol, 2 eq) were placed in 1000 ml of 1,4-dioxane and stirred at 100° C. for 9 hours. It was extracted with MC and water, and then the organic layer was dried over anhydrous Na₂SO₄ and filtered through silica gel. After it was precipitated with MC/MeOH, the precipitate was filtered to give 91 g of Compound 61-3 in a yield of 83%.

### 4) Preparation of Compound 61

Compound 1-4 (11.4 g, 21.86 mmol, 1 eq) and 2-bromo-1,10-phenanthroline (5.66 g, 21.86 mmol, 1 eq) were dissolved in 110 ml of 1,4-dioxane and 25 ml of distilled water, and then Pd(PPh₃)₄ (1.26 g, 1.09 mmol, 0.05 eq) and K₂CO₃ (6.04 g, 43.72 mmol, 2 eq) were placed therein and stirred under reflux for 15 hours. MC was placed and dissolved in the reaction solution, and then extracted with water, and the organic layer was dried over anhydrous Na₂SO₄. It was passed through a silica gel filter and then precipitated with MC/MeOH. The precipitated solid was filtered to give 9.0 g of Compound 61 in a yield of 72%.

Target compound B in Table 2 below was synthesized in the same manner as in Preparative Example 2, except that Intermediate B in Table 2 below was used instead of 2-bromo-1,10-phenanthroline.

TABLE 2

| Com- | | | |
|---|---|---|---|
| pound | | | |
| No. | Intermediate B | Target Compound B | Yield |
| 68 | 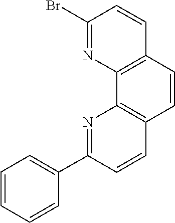 [SMILES: Brc1ccc2ccc3ccc(-c4ccccc4)nc3c2n1] | 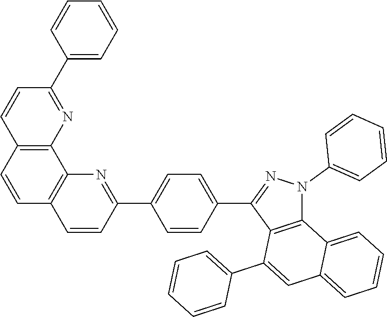 [SMILES: c1ccc(-c2ccc3ccc4ccc(-c5ccc(-c6nn(-c7ccccc7)c7c6c(-c6ccccc6)cc6ccccc67)cc5)nc4c3n2)cc1] | 87% |
| 69 | 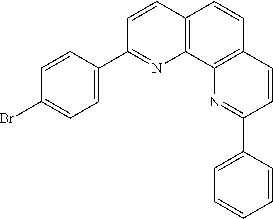 [SMILES: Brc1ccc(-c2ccc3ccc4ccc(-c5ccccc5)nc4c3n2)cc1] | 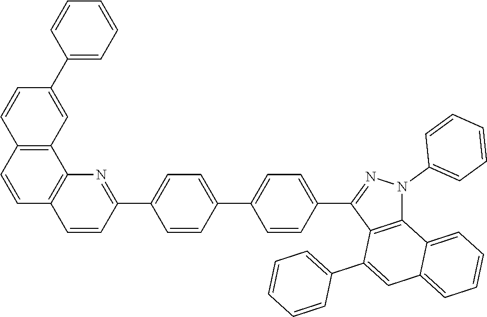 [SMILES: c1ccc(-c2ccc3ccc4ccc(-c5ccc(-c6ccc(-c7nn(-c8ccccc8)c8c7c(-c7ccccc7)cc7ccccc78)cc6)cc5)nc4c3c2)cc1] | 82% |
| 79 | 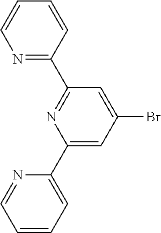 [SMILES: Brc1cc(-c2ccccn2)nc(-c2ccccn2)c1] | 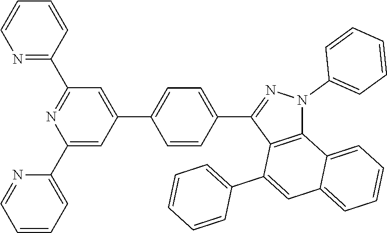 [SMILES: c1ccc(-c2cc3ccccc3c3c2c(-c2ccc(-c4cc(-c5ccccn5)nc(-c5ccccn5)c4)cc2)nn3-c2ccccc2)cc1] | 76% |
| 86 | 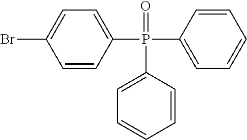 [SMILES: O=P(c1ccccc1)(c1ccccc1)c1ccc(Br)cc1] | 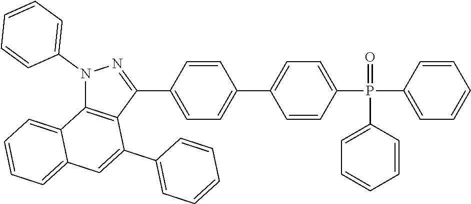 [SMILES: O=P(c1ccccc1)(c1ccccc1)c1ccc(-c2ccc(-c3nn(-c4ccccc4)c4c3c(-c3ccccc3)cc3ccccc34)cc2)cc1] | 73% |
| 87 | 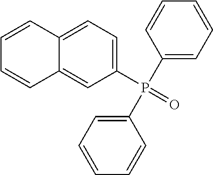 [SMILES: O=P(c1ccccc1)(c1ccccc1)c1ccc2ccccc2c1] | 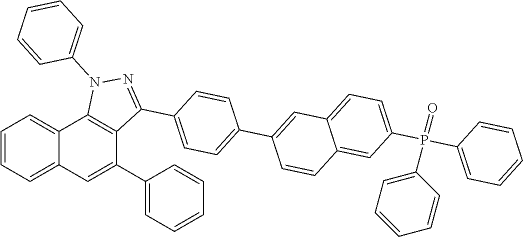 [SMILES: O=P(c1ccccc1)(c1ccccc1)c1ccc2cc(-c3ccc(-c4nn(-c5ccccc5)c5c4c(-c4ccccc4)cc4ccccc45)cc3)ccc2c1] | 75% |
| 88 | 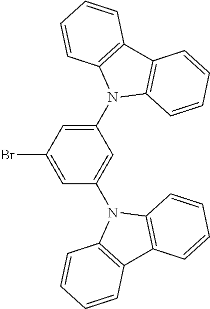 [SMILES: Brc1cc(-n2c3ccccc3c3ccccc32)cc(-n2c3ccccc3c3ccccc32)c1] | 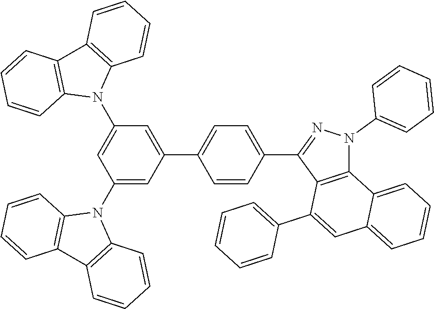 [SMILES: c1ccc(-c2cc3ccccc3c3c2c(-c2ccc(-c4cc(-n5c6ccccc6c6ccccc65)cc(-n5c6ccccc6c6ccccc65)c4)cc2)nn3-c2ccccc2)cc1] | 75% |
| 93 | 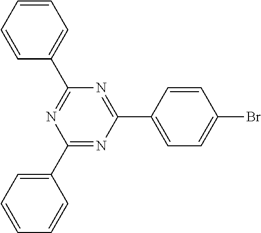 [SMILES: Brc1ccc(-c2nc(-c3ccccc3)nc(-c3ccccc3)n2)cc1] | 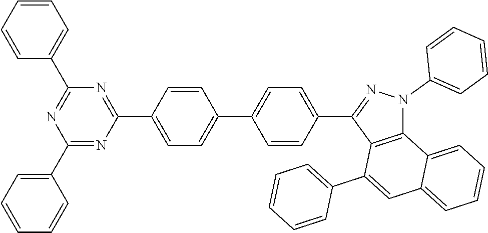 [SMILES: c1ccc(-c2nc(-c3ccccc3)nc(-c3ccc(-c4ccc(-c5nn(-c6ccccc6)c6c5c(-c5ccccc5)cc5ccccc56)cc4)cc3)n2)cc1] | 80% |
| 94 | 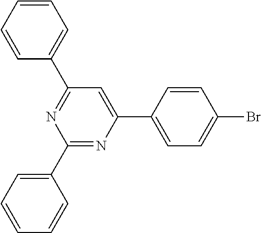 [SMILES: Brc1ccc(-c2cc(-c3ccccc3)nc(-c3ccccc3)n2)cc1] | 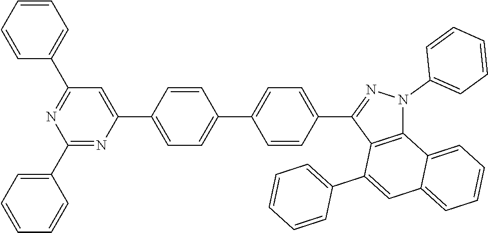 [SMILES: c1ccc(-c2cc(-c3ccc(-c4ccc(-c5nn(-c6ccccc6)c6c5c(-c5ccccc5)cc5ccccc56)cc4)cc3)nc(-c3ccccc3)n2)cc1] | 74% |
| 98 | 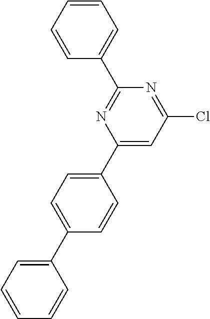 [SMILES: Clc1cc(-c2ccc(-c3ccccc3)cc2)nc(-c2ccccc2)n1] | 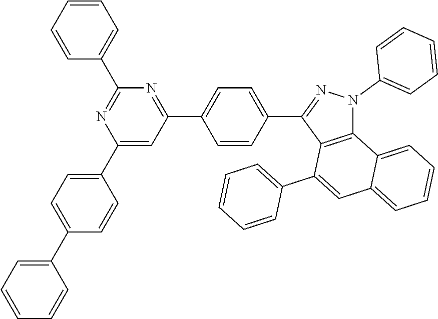 [SMILES: c1ccc(-c2ccc(-c3cc(-c4ccc(-c5nn(-c6ccccc6)c6c5c(-c5ccccc5)cc5ccccc56)cc4)nc(-c4ccccc4)n3)cc2)cc1] | 89% |
| 105 | 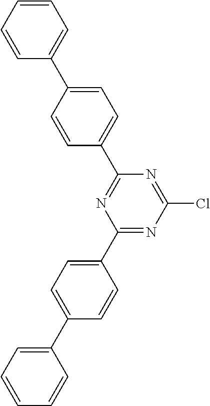 [SMILES: Clc1nc(-c2ccc(-c3ccccc3)cc2)nc(-c2ccc(-c3ccccc3)cc2)n1] | 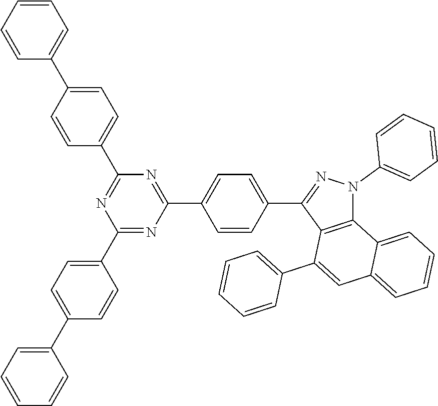 [SMILES: c1ccc(-c2ccc(-c3nc(-c4ccc(-c5ccccc5)cc4)nc(-c4ccc(-c5nn(-c6ccccc6)c6c5c(-c5ccccc5)cc5ccccc56)cc4)n3)cc2)cc1] | 78% |
| 112 | 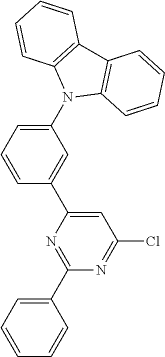 [SMILES: Clc1cc(-c2cccc(-n3c4ccccc4c4ccccc43)c2)nc(-c2ccccc2)n1] | 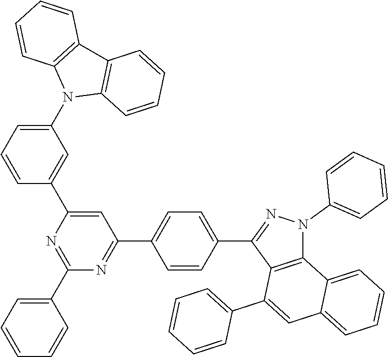 [SMILES: c1ccc(-c2nc(-c3ccc(-c4nn(-c5ccccc5)c5c4c(-c4ccccc4)cc4ccccc45)cc3)cc(-c3cccc(-n4c5ccccc5c5ccccc54)c3)n2)cc1] | 81% |
| 115 | 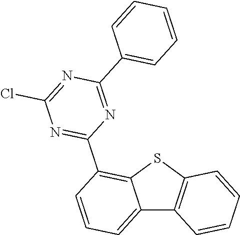 [SMILES: Clc1nc(-c2ccccc2)nc(-c2cccc3c2sc2ccccc23)n1] | 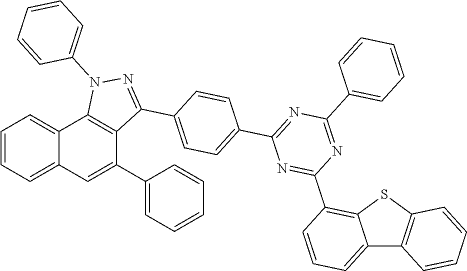 [SMILES: c1ccc(-c2nc(-c3ccc(-c4nn(-c5ccccc5)c5c4c(-c4ccccc4)cc4ccccc45)cc3)nc(-c3cccc4c3sc3ccccc34)n2)cc1] | 83% |
| 117 | 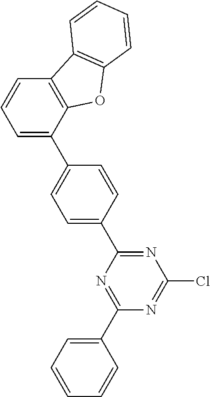 [SMILES: Clc1nc(-c2ccccc2)nc(-c2ccc(-c3cccc4c3oc3ccccc34)cc2)n1] | 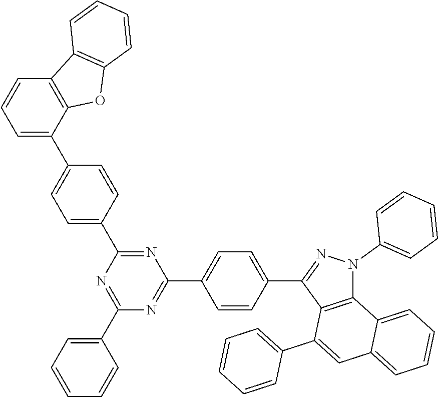 [SMILES: c1ccc(-c2nc(-c3ccc(-c4cccc5c4oc4ccccc45)cc3)nc(-c3ccc(-c4nn(-c5ccccc5)c5c4c(-c4ccccc4)cc4ccccc45)cc3)n2)cc1] | 79% |
| 120 | 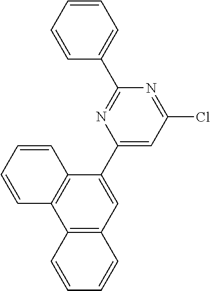 [SMILES: Clc1cc(-c2cc3ccccc3c3ccccc23)nc(-c2ccccc2)n1] | 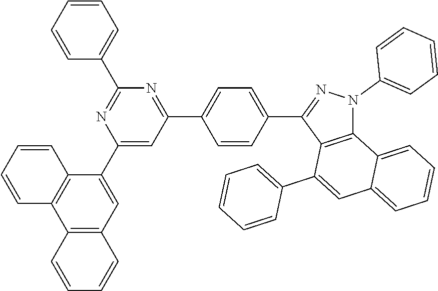 [SMILES: c1ccc(-c2nc(-c3ccc(-c4nn(-c5ccccc5)c5c4c(-c4ccccc4)cc4ccccc45)cc3)cc(-c3cc4ccccc4c4ccccc34)n2)cc1] | 68% |

### [Preparative Example 3] Preparation of Compound 121

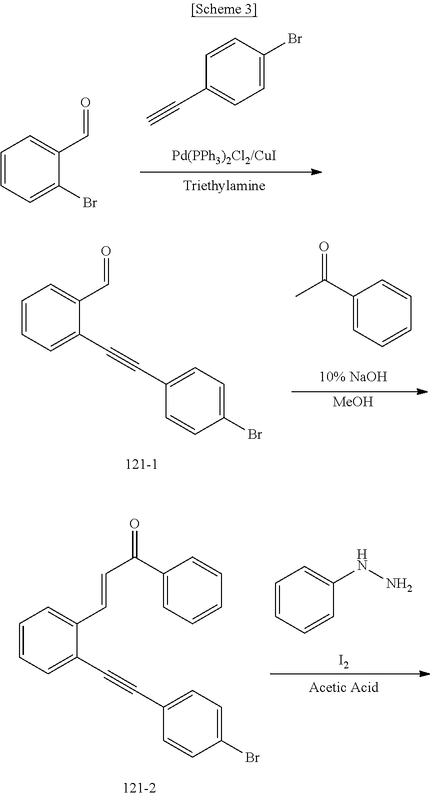

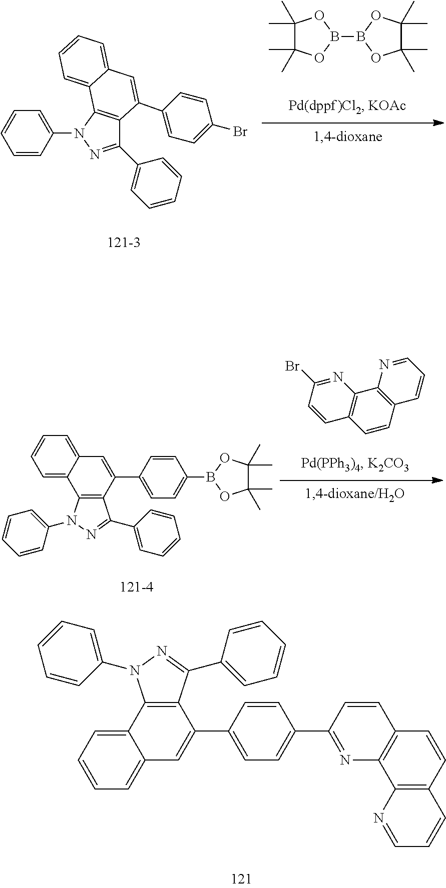

### 1) Preparation of Compound 121-1

2-bromobenzaldehyde (60 g, 324.29 mmol, 1 eq), 1-bromo-4-ethynylbenzene (64.6 g, 356.72 mmol, 1.1 eq), bis(triphenylphosphine)palladium(II) dichloride (4.56 g, 6.49 mmol, 0.02 eq), and copper iodide (0.61 g, 3.24 mmol, 0.01 eq) were placed in 600 ml of trimethylamine and stirred at 60° C. for 5 hours. The solution was passed through Celite and then washed with MC. The solvent was concentrated and then passed through silica gel. The solvent was removed to give 80.2 g of Compound 121-1 in a yield of 87%.

### 2) Preparation of Compound 121-2

Compound 121-1 (80.2 g, 281.23 mmol, 1 eq) and acetophenone (37.2 g, 309.35 mmol, 1.1 eq) were placed in 10% aqueous sodium hydroxide solution (58 ml) and methanol (580 ml) and stirred at room temperature for 8 hours. After completion of the reaction, the precipitated solid was filtered and washed with water and methanol. 90.4 g of compound 121-2 was obtained in a yield of 83%.

### 3) Preparation of Compound 121-3

Compound 121-2 (90.4 g, 233.48 mmol, 1 eq) was added to acetic acid (720 ml), and then phenylhydrazine (30.3 g, 280.18 mmol, 1.2 eq) and iodine (71.1 g, 280.18) mmol, 1.2 eq) were placed therein with stirring and stirred under reflux for 13 hours. After completion of the reaction, the reaction solution was cooled to room temperature and diluted with distilled water, and then neutralized with an aqueous sodium hydrogen carbonate solution. It was extracted with ethyl acetate and distilled water, and then separated by a silica gel column (developing solvent MC:Hex=1:5) to give 100 g of compound 121-3 in a yield of 75%.

### 4) Preparation of Compound 121-4

Compound 121-3 (100 g, 210.35 mmol, 1 eq), bis(pinacolato)diboron (80.1 g, 315.52 mmol, 1.5 eq), PdCl₂(dppf) (15.39 g, 21.04 mmol, 0.1 eq), and KOAc (41.29 g, 420.70 mmol, 2 eq) were placed in 1000 ml of 1,4-dioxane and stirred at 100° C. for 12 hours. It was extracted with MC and water, and then the organic layer was dried over anhydrous Na₂SO₄ and filtered through silica gel. After it was precipitated with MC/MeOH, the precipitate was filtered to give 85.7 g of Compound 121-4 in a yield of 78%.

### 5) Preparation of Compound 121

Compound 121-4 (11.4 g, 21.86 mmol, 1 eq) and 2-bromo-1,10-phenanthroline (5.66 g, 21.86 mmol, 1 eq) were dissolved in 110 ml of 1,4-dioxane and 25 ml of distilled water, and then Pd(PPh₃)₄ (1.26 g, 1.09 mmol, 0.05 eq) and K₂CO₃ (6.04 g, 43.72 mmol, 2 eq) were placed therein and stirred under reflux for 15 hours. MC was placed and dissolved in the reaction solution, and then extracted with water, and the organic layer was dried over anhydrous Na₂SO₄. It was passed through a silica gel filter and precipitated with MC/MeOH. The precipitated solid was filtered to give 8.8 g of Compound 121 in a yield of 70%.

Target compound C in Table 3 was synthesized in the same manner as in Preparative Example 3, except that Intermediate C in Table 3 below was used instead of 2-bromo-1,10-phenanthroline.

TABLE 3

| Com- | | | |
|---|---|---|---|
| pound | | | |
| No. | Intermediate C | Target Compound C | Yield |
| 129 | 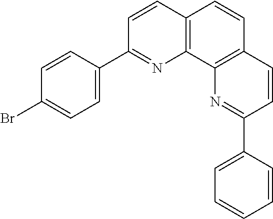 [SMILES: Brc1ccc(-c2ccc3ccc4ccc(-c5ccccc5)nc4c3n2)cc1] | 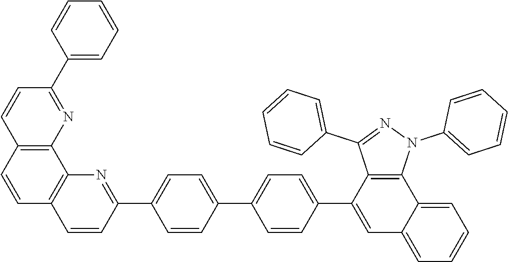 [SMILES: c1ccc(-c2ccc3ccc4ccc(-c5ccc(-c6ccc(-c7cc8ccccc8c8c7c(-c7ccccc7)nn8-c7ccccc7)cc6)cc5)nc4c3n2)cc1] | 91% |
| 146 | 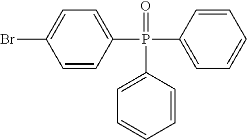 [SMILES: O=P(c1ccccc1)(c1ccccc1)c1ccc(Br)cc1] | 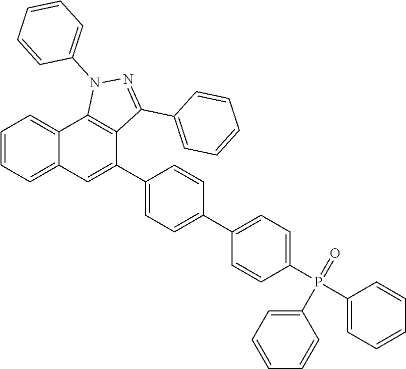 [SMILES: O=P(c1ccccc1)(c1ccccc1)c1ccc(-c2ccc(-c3cc4ccccc4c4c3c(-c3ccccc3)nn4-c3ccccc3)cc2)cc1] | 69% |
| 148 | 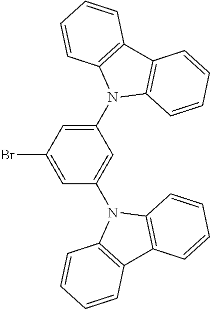 [SMILES: Brc1cc(-n2c3ccccc3c3ccccc32)cc(-n2c3ccccc3c3ccccc32)c1] | 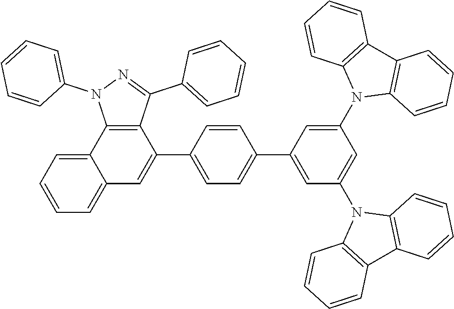 [SMILES: c1ccc(-c2nn(-c3ccccc3)c3c2c(-c2ccc(-c4cc(-n5c6ccccc6c6ccccc65)cc(-n5c6ccccc6c6ccccc65)c4)cc2)cc2ccccc23)cc1] | 77% |
| 149 | 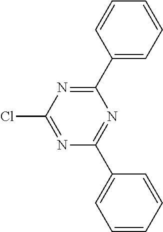 [SMILES: Clc1nc(-c2ccccc2)nc(-c2ccccc2)n1] | 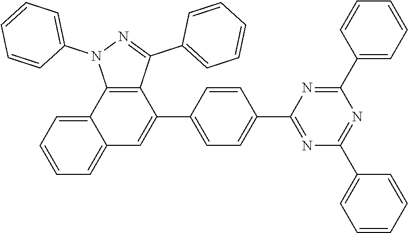 [SMILES: c1ccc(-c2nc(-c3ccccc3)nc(-c3ccc(-c4cc5ccccc5c5c4c(-c4ccccc4)nn5-c4ccccc4)cc3)n2)cc1] | 69% |
| 150 | 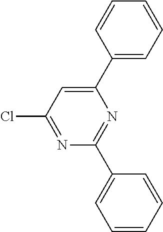 [SMILES: Clc1cc(-c2ccccc2)nc(-c2ccccc2)n1] | 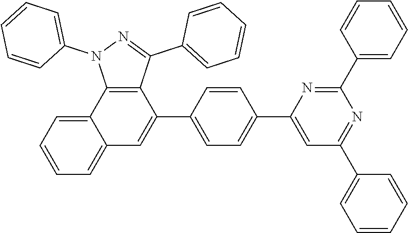 [SMILES: c1ccc(-c2cc(-c3ccc(-c4cc5ccccc5c5c4c(-c4ccccc4)nn5-c4ccccc4)cc3)nc(-c3ccccc3)n2)cc1] | 75% |
| 154 | 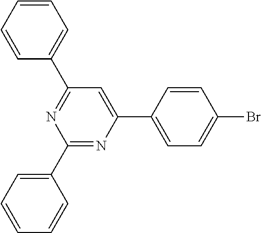 [SMILES: Brc1ccc(-c2cc(-c3ccccc3)nc(-c3ccccc3)n2)cc1] | 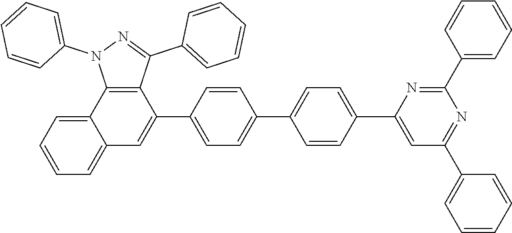 [SMILES: c1ccc(-c2cc(-c3ccc(-c4ccc(-c5cc6ccccc6c6c5c(-c5ccccc5)nn6-c5ccccc5)cc4)cc3)nc(-c3ccccc3)n2)cc1] | 71% |
| 159 | 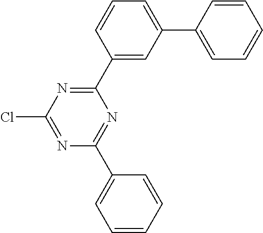 [SMILES: Clc1nc(-c2ccccc2)nc(-c2cccc(-c3ccccc3)c2)n1] | 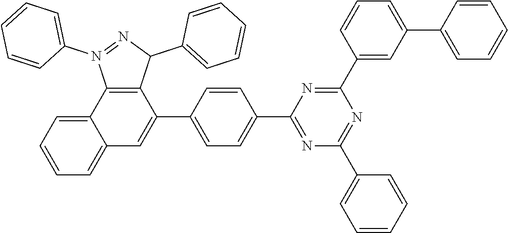 | 83% |
| 161 | 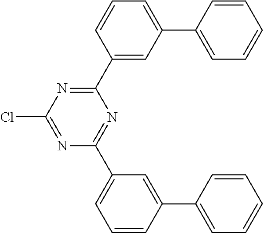 [SMILES: Clc1nc(-c2cccc(-c3ccccc3)c2)nc(-c2cccc(-c3ccccc3)c2)n1] | 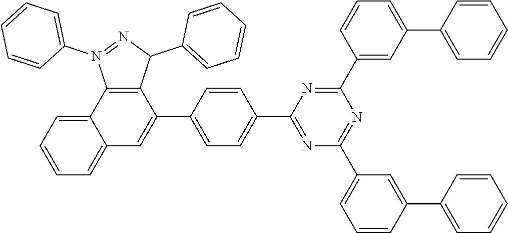 | 77% |
| 164 | 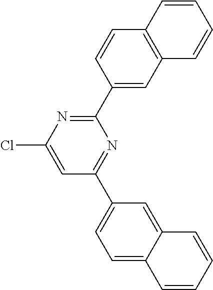 [SMILES: Clc1cc(-c2ccc3ccccc3c2)nc(-c2ccc3ccccc3c2)n1] | 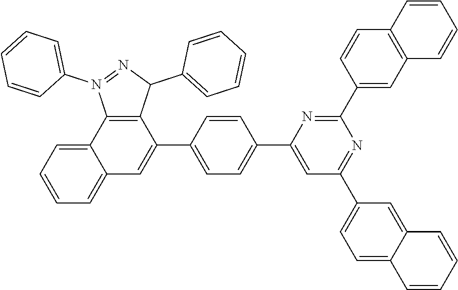 | 82% |
| 167 | 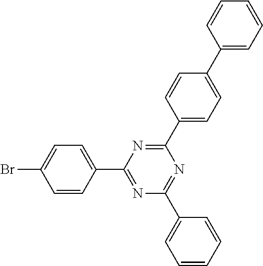 [SMILES: Brc1ccc(-c2nc(-c3ccccc3)nc(-c3ccc(-c4ccccc4)cc3)n2)cc1] | 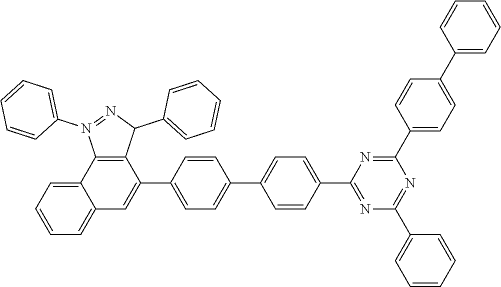 | 63% |
| 178 | 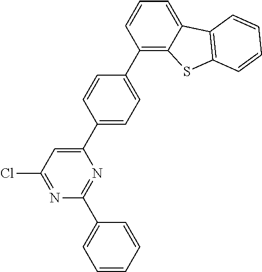 [SMILES: Clc1cc(-c2ccc(-c3cccc4c3sc3ccccc34)cc2)nc(-c2ccccc2)n1] | 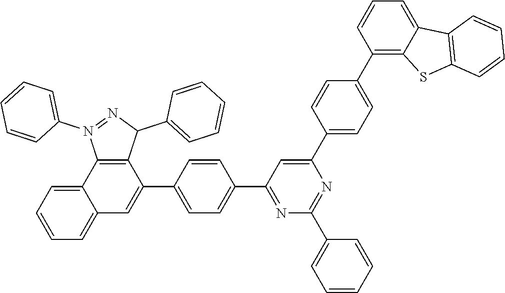 | 71% |

### [Preparative Example 4] Preparation of Compound 321

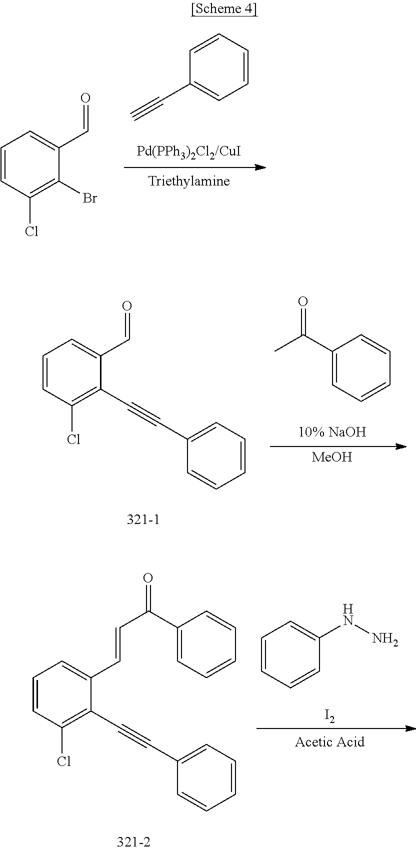

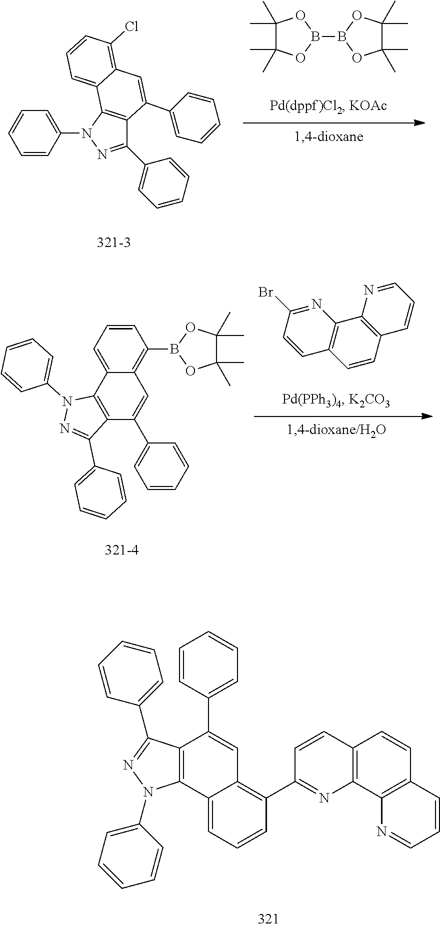

### 1) Preparation of Compound 321-1

2-bromo-3-chlorobenzaldehyde (71.2 g, 324.29 mmol, 1 eq), ethynylbenzene (36.4 g, 356.72 mmol, 1.1 eq), bis(triphenylphosphine)palladium(II) dichloride (4.56 g, 6.49 mmol, 0.02 eq), and copper iodide (0.61 g, 3.24 mmol, 0.01 eq) were placed in 700 ml of trimethylamine and stirred at 60° C. for 8 hours. The solution was passed through Celite and then washed with MC. The solvent was concentrated and then passed through silica gel. The solvent was removed to give 67.7 g of Compound 321-1 in a yield of 87%.

### 2) Preparation of Compound 321-2

Compound 321-1 (67.7 g, 281.23 mmol, 1 eq) and acetophenone (37.2 g, 309.35 mmol, 1.1 eq) were placed in 10% aqueous sodium hydroxide solution (67 ml) and methanol (670 ml) and stirred at room temperature for 10 hours. After completion of the reaction, the precipitated solid was filtered and washed with water and methanol. 80 g of compound 321-2 was obtained in a yield of 83%.

### 3) Preparation of Compound 321-3

Compound 321-2 (80 g, 233.48 mmol, 1 eq) was added to acetic acid (800 ml), and then phenylhydrazine (30.3 g, 280.18 mmol, 1.2 eq) and iodine (71.1 g, 280.18) mmol, 1.2 eq) were placed therein with stirring and stirred under reflux for 16 hours. After completion of the reaction, the reaction solution was cooled to room temperature and diluted with distilled water, and then neutralized with an aqueous sodium hydrogen carbonate solution. It was extracted with ethyl acetate and distilled water, and then separated by a silica gel column (developing solvent MC:Hex=1:5) to give 90.6 g of compound 321-3 in a yield of 75%.

### 4) Preparation of Compound 321-4

Compound 321-3 (90.6 g, 210.35 mmol, 1 eq), bis(pinacolato)diboron (80.1 g, 315.52 mmol, 1.5 eq), PdCl₂(dppf) (15.39 g, 21.04 mmol, 0.1 eq), and KOAc (41.29 g, 420.70 mmol, 2 eq) were placed in 900 ml of 1,4-dioxane and stirred at 100° C. for 18 hours. It was extracted with MC and water, and then the organic layer was dried over anhydrous Na₂SO₄ and filtered through silica gel. After it was precipitated with MC/MeOH, the precipitate was filtered to give 77.6 g of Compound 321-4 in a yield of 85%.

### 5) Preparation of Compound 321

Compound 321-4 (11.4 g, 21.86 mmol, 1 eq) and 2-bromo-1,10-phenanthroline (5.66 g, 21.86 mmol, 1 eq) were dissolved in 110 ml of 1,4-dioxane and 25 ml of distilled water, and then Pd(PPh₃)₄ (1.26 g, 1.09 mmol, 0.05 eq) and K₂CO₃ (6.04 g, 43.72 mmol, 2 eq) were placed therein and stirred under reflux for 8 hours. MC was placed and dissolved in the reaction solution, and then extracted with water, and the organic layer was dried over anhydrous Na₂SO₄. It was passed through a silica gel filter and precipitated with MC/MeOH. The precipitated solid was filtered to give 9.7 g of Compound 321 in a yield of 77%.

Target compound E in Table 4 below was synthesized in the same manner as in Preparative Example 4, except that Intermediate D instead of 2-bromo-3-chlorobenzaldehyde and Intermediate E instead of 2-bromo-1,10-phenanthroline in Table 4 below were used.

TABLE 4

| Com- | | | | |
|---|---|---|---|---|
| pound | | | | |
| No. | Intermediate D | Intermediate E | Target Compound E | Yield |
| 322 | 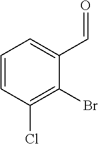 | 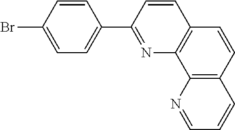 [SMILES: Brc1ccc(-c2ccc3ccc4cccnc4c3n2)cc1] | 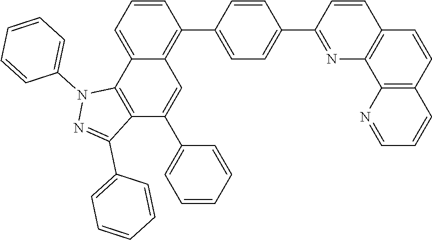 [SMILES: c1ccc(-c2cc3c(-c4ccc(-c5ccc6ccc7cccnc7c6n5)cc4)cccc3c3c2c(-c2ccccc2)nn3-c2ccccc2)cc1] | 91% |
| 346 | 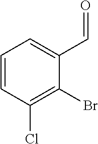 | 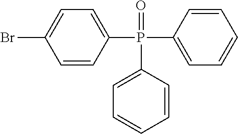 [SMILES: O=P(c1ccccc1)(c1ccccc1)c1ccc(Br)cc1] | 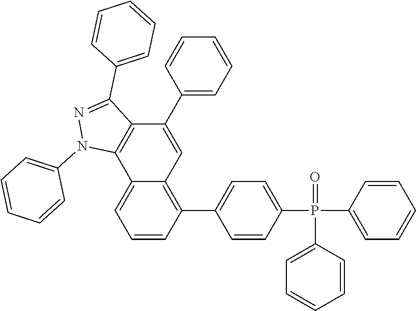 [SMILES: O=P(c1ccccc1)(c1ccccc1)c1ccc(-c2cccc3c2cc(-c2ccccc2)c2c(-c4ccccc4)nn(-c4ccccc4)c23)cc1] | 69% |
| 366 | 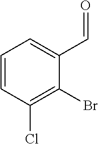 | 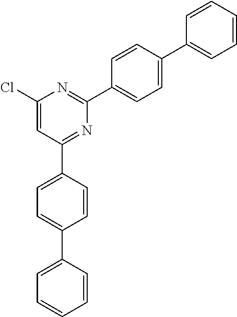 [SMILES: Clc1cc(-c2ccc(-c3ccccc3)cc2)nc(-c2ccc(-c3ccccc3)cc2)n1] | 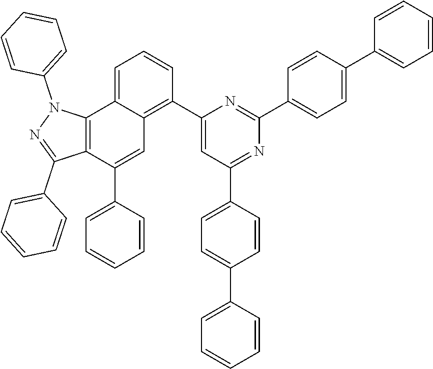 [SMILES: c1ccc(-c2ccc(-c3cc(-c4cccc5c4cc(-c4ccccc4)c4c(-c6ccccc6)nn(-c6ccccc6)c45)nc(-c4ccc(-c5ccccc5)cc4)n3)cc2)cc1] | 77% |
| 371 | 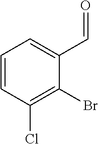 | 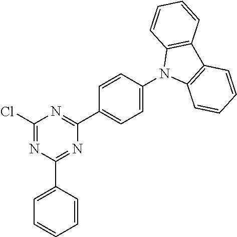 [SMILES: Clc1nc(-c2ccccc2)nc(-c2ccc(-n3c4ccccc4c4ccccc43)cc2)n1] | 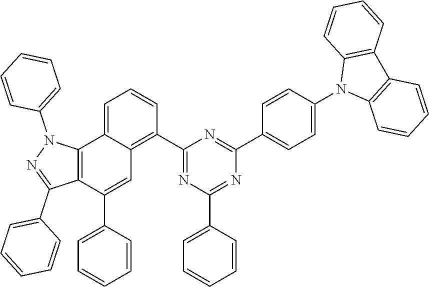 [SMILES: c1ccc(-c2nc(-c3ccc(-n4c5ccccc5c5ccccc54)cc3)nc(-c3cccc4c3cc(-c3ccccc3)c3c(-c5ccccc5)nn(-c5ccccc5)c34)n2)cc1] | 69% |
| 389 | 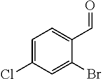 | 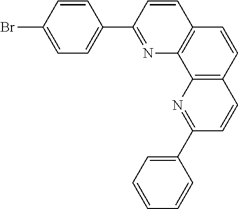 [SMILES: Brc1ccc(-c2ccc3ccc4ccc(-c5ccccc5)nc4c3n2)cc1] | 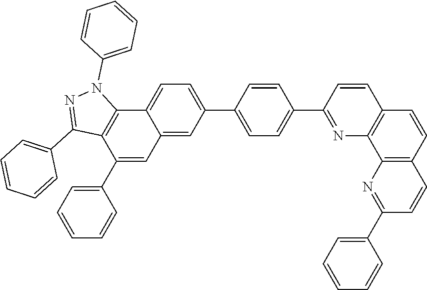 [SMILES: c1ccc(-c2ccc3ccc4ccc(-c5ccc(-c6ccc7c(c6)cc(-c6ccccc6)c6c(-c8ccccc8)nn(-c8ccccc8)c67)cc5)nc4c3n2)cc1] | 75% |
| 407 | 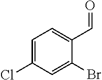 | 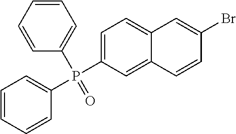 [SMILES: O=P(c1ccccc1)(c1ccccc1)c1ccc2cc(Br)ccc2c1] | 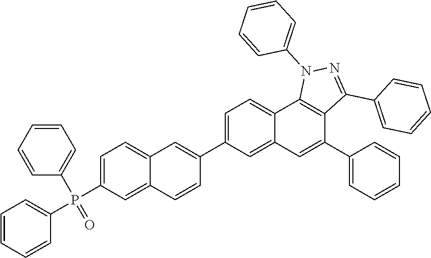 [SMILES: O=P(c1ccccc1)(c1ccccc1)c1ccc2cc(-c3ccc4c(c3)cc(-c3ccccc3)c3c(-c5ccccc5)nn(-c5ccccc5)c34)ccc2c1] | 71% |
| 414 | 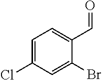 | 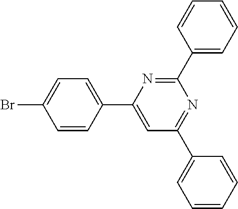 [SMILES: Brc1ccc(-c2cc(-c3ccccc3)nc(-c3ccccc3)n2)cc1] | 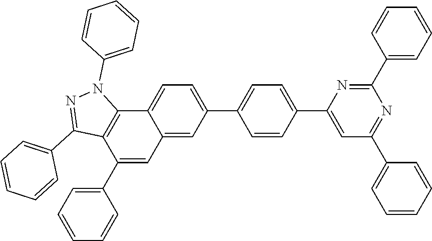 [SMILES: c1ccc(-c2cc(-c3ccc(-c4ccc5c(c4)cc(-c4ccccc4)c4c(-c6ccccc6)nn(-c6ccccc6)c45)cc3)nc(-c3ccccc3)n2)cc1] | 83% |
| 439 | 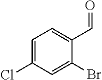 | 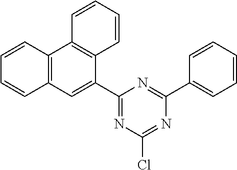 [SMILES: Clc1nc(-c2ccccc2)nc(-c2cc3ccccc3c3ccccc23)n1] | 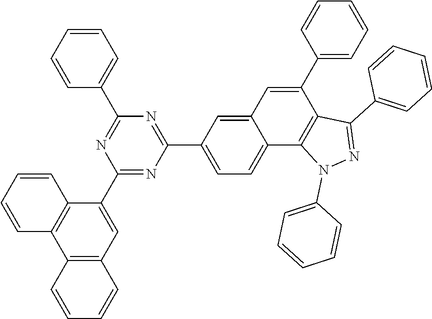 [SMILES: c1ccc(-c2nc(-c3ccc4c(c3)cc(-c3ccccc3)c3c(-c5ccccc5)nn(-c5ccccc5)c34)nc(-c3cc4ccccc4c4ccccc34)n2)cc1] | 77% |
| 473 | 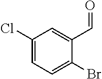 | 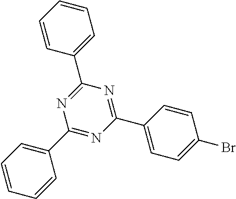 [SMILES: Brc1ccc(-c2nc(-c3ccccc3)nc(-c3ccccc3)n2)cc1] | 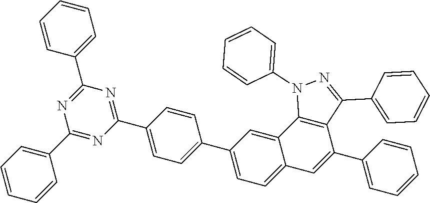 [SMILES: c1ccc(-c2nc(-c3ccccc3)nc(-c3ccc(-c4ccc5cc(-c6ccccc6)c6c(-c7ccccc7)nn(-c7ccccc7)c6c5c4)cc3)n2)cc1] | 82% |
| 477 | 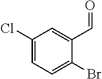 | 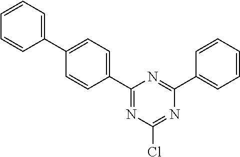 [SMILES: Clc1nc(-c2ccccc2)nc(-c2ccc(-c3ccccc3)cc2)n1] | 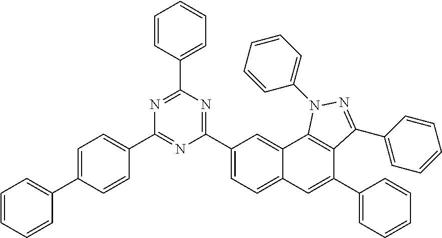 [SMILES: c1ccc(-c2ccc(-c3nc(-c4ccccc4)nc(-c4ccc5cc(-c6ccccc6)c6c(-c7ccccc7)nn(-c7ccccc7)c6c5c4)n3)cc2)cc1] | 63% |
| 502 | 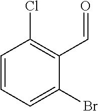 | 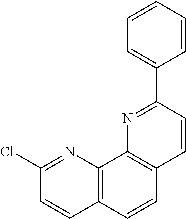 [SMILES: Clc1ccc2ccc3ccc(-c4ccccc4)nc3c2n1] | 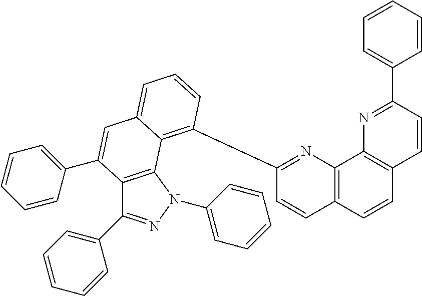 [SMILES: c1ccc(-c2ccc3ccc4ccc(-c5cccc6cc(-c7ccccc7)c7c(-c8ccccc8)nn(-c8ccccc8)c7c56)nc4c3n2)cc1] | 71% |

Compounds were prepared in the same manner as in the above preparative examples and the synthesis confirmation results are shown in Tables 5 and 6. Table 5 shows the measured values of ¹H NMR (CDCl₃, 200 Mz) and Table 6 shows the measured values of field desorption mass spectrometry (FD-MS).

TABLE 5

| Compound | ¹H NMR(CDCl₃, 200 Mz) |
|---|---|
| 1 | δ = 8.80(d, 1H), 8.71(d, 1H), 8.45(d, 1H), 8.20-8.16(m, |
| | 3H), 8.02-7.79(m, 9H), 7.59-7.41(m, 10H), 7.29(d, 1H) |
| 8 | δ = 8.71(d, 2H), 8.33(d, 2H), 8.20-8.16(m, 3H), 8.02- |
| | 7.79(m, 9H), 7.59-7.40(m, 12H), 7.29(d, 2H) |
| 9 | δ = 8.71-8.69(m, 4H), 8.33(d, 2H), 8.20(d, 1H), 8.02- |
| | 7.98(m, 2H), 7.90-7.79(m, 11H), 7.59-7.40(m, 12H), |
| | 7.29(d, 2H) |
| 26 | δ = 8.02-7.97(m, 6H), 7.84-7.77(m, 12H), 7.59-7.40(m, |
| | 15H) |
| 28 | δ = 8.55(d, 2H), 8.19-8.17(m, 4H), 8.02-7.94(m, 4H), |
| | 7.84-7.77(m, 8H), 7.59-7.35(m, 16H), 7.20-7.16(m, 4H) |
| 29 | δ = 8.36(d, 4H), 8.02(d, 1H), 7.98(d, 1H), 7.84-7.77(m, |
| | 8H), 7.59-7.41(m, 15H) |
| 30 | δ = 8.35(d, 2H), 8.23(s, 1H), 7.98-7.94(m, 4H), 7.84- |
| | 7.77(m, 8H), 7.59-7.40(m, 15H) |
| 37 | δ = 8.36(d, 2H), 8.02-7.96(m, 4H), 7.84-7.75(m, 10H), |
| | 7.59-7.41(m, 15H), 7.25(d, 2H) |
| 38 | δ = 8.23(s, 1H), 8.02-7.94(m, 6H), 7.84-7.75 m, 10H), |
| | 7.55-7.40(m, 15H), 7.25(d, 2H) |
| 39 | δ = 8.38-8.36(m, 3H), 8.02-7.94(m, 3H), 7.84-7.73(m, |
| | 11H), 7.61-7.41(m, 16H) |
| 46 | δ = 8.30(d, 2H), 8.02-7.96(m, 4H), 7.85-7.75(m, 14H), |
| | 7.59-7.41(m, 15H), 7.25(d, 2H) |
| 61 | δ = 8.80(d, 1H), 8.71-8.69(m, 3H), 8.45(d, 1H), 8.30(d, |
| | 2H), 8.20(d, 1H), 8.02-7.98(m, 2H), 7.90(d, 1H), |
| | 7.79(d, 2H), 7.60-7.41(m, 12H), 7.29(d, 1H) |
| 68 | δ = 8.71(d, 2H), 8.69(d, 2H), 8.33-8.30(m, 4H), 8.20(d, |
| | 1H), 8.02-7.98(m, 2H), 7.90(d, 1H), 7.79(d, 2H), 7.60- |
| | 7.41(m, 14H), 7.29(d, 2H) |
| 69 | δ = 8.71-8.69(m, 4H), 8.33-8.30(m, 4H), 8.20(d, 1H), |
| | 8.02-7.98(m, 2H), 7.90-7.79(m, 7H), 7.60-7.40(m, 14H), |
| | 7.29(d, 2H) |
| 79 | δ = 9.18-9.14(m, 4H), 8.55(d, 2H), 8.30(d, 2H), 8.02- |
| | 7.98(m, 2H), 7.85-7.74(m, 6H), 7.60-7.40(m, 11H), |
| | 7.23(t, 2H) |
| 86 | δ = 8.30(d, 2H), 8.02-7.96(m, 6H), 7.85-7.77(m, 8H), |
| | 7.60-7.41(m, 17H) |
| 87 | δ = 8.43(s, 1H), 8.30(d, 2H), 8.16(d, 1H), 8.03-7.98(m, |
| | 4H), 7.85-7.77(m, 8H), 7.60-7.38(m, 19H) |
| 88 | δ = 8.55(d, 2H), 8.30(d, 2H), 8.19-8.17(m, 4H), 8.02- |
| | 7.94(m, 4H), 7.85-7.79(, 4H), 7.60-7.35(m, 18H), 7.20- |
| | 7.16(m, 4H) |
| 93 | δ = 8.36(d, 4H), 8.30(d, 2H), 8.02-7.96(m, 4H), 7.85- |
| | 7.79(m, 4H), 7.60-7.40(m, 17H), 7.25(d, 2H) |
| 94 | δ = 8.35-8.30(m, 6H), 8.02-7.94(m, 4H), 7.85-7.79(m, |
| | 6H), 7.60-7.40(m, 17H) |
| 98 | δ = 8.35-8.30(m, 8H), 8.23(s, 1H), 8.02-7.98(m, 2H), |
| | 7.85-7.75(m, 6H), 7.60-7.40(m, 17H) |
| 105 | δ = 8.30(d, 2H), 8.02-7.96(m, 8H), 7.79-7.75(m, 6H), |
| | 7.60-7.40(m, 17H), 7.25(d, 4H) |
| 112 | δ = 8.55(d, 1H), 8.35-8.30(m, 6H), 8.23-8.19(m, 3H), |
| | 8.02-7.94(m, 3H), 7.80-7.79(m, 3H), 7.68-7.35(m, 19H), |
| | 7.20-7.16(m, 2H) |
| 115 | δ = 8.55(d, 1H), 8.45(d, 1H), 8.36-8.30(m, 4H), 8.02- |
| | 7.92(m, 6H), 7.79(m, 2H), 7.70(t, 1H), 7.60-7.40(m, |
| | 17H) |
| 117 | δ = 8.36-8.30(m, 4H), 8.02-7.96(m, 9H), 7.79(d, 2H), |
| | 7.60-7.25(m, 20H) |
| 120 | δ = 9.08(d, 1H), 8.84(d, 1H), 8.35-8.30(m, 6H), 8.23(s, |
| | 1H), 8.17(d, 1H), 8.05(s, 1H), 8.02-7.98(m, 2H), 7.70- |
| | 7.40(m, 20H) |
| 121 | δ = 8.80(d, 1H), 8.71-8.69(m, 3H), 8.45(d, 1H), 8.20(d, |
| | 1H), 8.02-7.98(m, 2H), 7.90-7.84(m, 3H), 7.60-7.49(m, |
| | 11H), 7.40(s, 1H), 7.29-7.25(m, 3H) |
| 129 | δ = 8.71-8.69(m, 4H), 8.33(d, 2H), 8.20(d, 1H), 8.02- |
| | 7.98(m, 2H), 7.90-7.84(m, 5H), 7.60-7.49(m, 13H), 7.29- |
| | 7.25(m, 6H) |
| 146 | δ = 8.02-7.96(m, 6H), 7.84-7.77(m, 6H), 7.60-7.51(m, |
| | 16H), 7.40(s, 1H), 7.25(d, 4H) |
| 148 | δ = 8.55(d, 2H), 8.19-8.17(m, 4H), 8.02-7.94(m, 4H), |
| | 7.84(d, 2H), 7.60-7.49(m, 15H), 7.40-7.35(m, 3H), 7.25- |
| | 7.16(m, 8H) |
| 149 | δ = 8.36(d, 4H), 8.02-7.96(m, 4H), 7.84(d, 2H), 7.60- |
| | 7.49(m, 16H), 7.40(s, 1H), 7.25(d, 2H) |
| 150 | δ = 8.35-8.30(m, 4H), 8.23(s, 1H), 8.02-7.94(m, 4H), |
| | 7.84(d, 2H), 7.60-7.49(m, 16H), 7.25(d, 2H) |
| 154 | δ = 8.35-8.30(m, 4H), 8.23(s, 1H), 8.02-7.94(m, 4H), |
| | 7.85-7.84(m, 4H), |
| | 7.60-7.49(m, 16H), 7.40(s, 1H), 7.25(d, 4H) |
| 159 | δ = 8.38-8.36(m, 3H), 8.02-7.94(m, 5H), 7.84(d, 2H), |
| | 7.75-7.73(m, 3H), 7.61-7.41(m, 18H), 7.25(d, 2H) |
| 161 | δ = 8.38(d, 2H), 8.02-7.94(m, 6H), 7.84(d, 2H), 7.75- |
| | 7.73(m, 6H), 7.61-7.41(m, 19H), 7.25(d, 2H) |
| 164 | δ = 9.09(s, 1H), 8.49-8.46(m, 2H), 8.30(d, 2H), 8.23(s, |
| | 1H), 8.08-7.98(m, 8H), 7.84(d, 2H), 7.61-7.49(m, 14H), |
| | 7.40(s, 1H), 7.25(d, 2H) |
| 167 | δ = 8.36(d, 2H), 8.02-7.96(m, 6H), 7.84(d, 2H), 7.75(d, |
| | 2H), 7.60-7.40(m, 17H), 7.25(d, 8H) |
| 178 | δ = 8.55(d, 1H), 8.45(d, 1H), 8.35-8.30(m, 7H), 8.23(s, |
| | 1H), 8.02-7.93(m, 3H), 7.84(d, 2H), 7.70(t, 1H), 7.60- |
| | 7.49(m, 15H), 7.40(s, 1H), 7.25(d, 4H) |
| 321 | δ = 8.80(d, 1H), 8.71(d, 1H), 8.49-8.45(m, 2H), 8.22- |
| | 8.20(m, 2H), 8.00(t, 1H), 7.90-7.79(m, 5H), 7.60- |
| | 7.40(m, 13H), 7.29(d, 1H) |
| 322 | δ = 8.80(d, 1H), 8.71-8.69(m, 3H), 8.50-8.45(m, 2H), |
| | 8.20(d, 1H), 8.06(d, 1H), 7.90-7.77(m, 6H), 7.60- |
| | 7.40(m, 13H), 7.29-7.25(m, 3H) |
| 346 | δ = 8.50(d, 1H), 8.06(d, 1H), 7.97(d, 4H), 7.84-7.77(m, |
| | 9H), 7.60-7.40(m, 18H) |
| 366 | δ = 8.30(d, 2H), 8.23-8.22(m, 2H), 8.10(d, 1H), 8.00- |
| | 7.96(m, 3H), 7.85-7.75(m, 10H), 7.60-7.40(m, 18H), |
| | 7.25(d, 2H) |
| 371 | δ = 8.55(d, 1H), 8.36(d, 2H), 8.22-8.19(m, 2H), 8.10(d, |
| | 1H), 8.00-7.84(m, 10H), 7.60-7.35(m, 18H), 7.20(t, 1H), |
| | 7.16(t, 1H) |
| 389 | δ = 8.71-8.69(m, 4H), 8.33(d, 2H), 8.23(d, 1H), 8.20(d, |
| | 1H), 7.90-7.79(m, 7H), 7.60-7.40(m, 17H), 7.29(d, 2H) |
| 407 | δ = 8.43(s, 1H), 8.23(d, 2H), 8.16(d, 1H), 8.03-7.99(m, |
| | 2H), 7.84-7.77(m, 8H), 7.60-7.38(m, 22H) |
| 414 | δ = 8.35-8.23(m, 6H), 7.94(d, 2H), 7.85-7.79(m, 6H), |
| | 7.60-7.38(m, 20H) |
| 439 | δ = 9.09-9.08(m, 2H), 8.84(d, 1H), 8.49(d, 1H), 8.36(d, |
| | 2H), 8.17(d, 1H), 8.05(s, 1H), 7.70-7.40(m, 24H) |
| 473 | δ = 8.36-8.32(m, 5H), 7.99-7.96(m, 3H), 7.84-7.79(m, |
| | 4H), 7.60-7.38(m, 19H), 7.25(d, 2H) |
| 477 | δ = 8.49-8.46(m, 2H), 8.36(d, 2H), 7.96-7.93(m, 3H), |
| | 7.84-7.75(m, 6H), 7.60-7.40(m, 18H), 7.25(d, 2H) |
| 502 | δ = 8.71(d, 2H), 8.49(d, 1H), 8.33-8.20(m, 4H), 8.00(t, |
| | 1H), 7.90-7.79(5H, m), 7.60-7.40(m, 15H), 7.29(d, 2H) |

TABLE 6

| Compound | FD-MS | Compound | FD-MS |
|---|---|---|---|
| 1 | m/z = 509.60 | 8 | m/z = 585.70 |
| | (C37H23N3 = 509.19) | | (C43H27N3 = 585.22) |
| 9 | m/z = 726.88 | 26 | m/z = 672.77 |
| | (C53H34N4 = 726.28) | | (C47H33N2OP = 672.23) |
| 28 | m/z = 802.98 | 29 | m/z = 627.75 |
| | (C59H38N4 = 802.31) | | (C44H29N5 = 627.24) |
| 30 | m/z = 626.76 | 37 | m/z = 703.85 |
| | (C45H30N4 = 626.25) | | (C50H33N5 = 703.27) |
| 38 | m/z = 702.86 | 39 | m/z = 703.85 |
| | (C51H34N4 = 702.28) | | (C50H33N5 = 703.27) |
| 46 | m/z = 778.96 | 61 | m/z = 574.69 |
| | (C57H38N4 = 778.31) | | (C41H26N4 = 574.22) |
| 68 | m/z = 650.78 | 69 | m/z = 726.88 |
| | (C47H30N4 = 650.25) | | (C53H34N4 = 726.28) |
| 79 | m/z = 627.75 | 86 | m/z = 672.77 |
| | (C44H29N5 = 627.24) | | (C47H33N2OP = 672.23) |
| 87 | m/z = 722.83 | 88 | m/z = 802.98 |
| | (C51H35N2OP = 722.25) | | (C59H38N4 = 802.31) |
| 93 | m/z = 703.85 | 94 | m/z = 702.86 |
| | (C50H33N5 = 703.27) | | (C51H34N4 = 702.28) |
| 98 | m/z = 702.86 | 105 | m/z = 779.95 |
| | (C51H34N4 = 702.28) | | (C56H37N5 = 779.30) |
| 112 | m/z = 791.96 | 115 | m/z = 733.89 |
| | (C57H37N5 = 791.30) | | (C50H31N5S = 733.23) |
| 117 | m/z = 793.93 | 120 | m/z = 726.88 |
| | (C56H35N50 = 793.28) | | (C53H34N4 = 726.28) |
| 121 | m/z = 574.69 | 129 | m/z = 726.88 |
| | (C41H26N4 = 574.22) | | (C53H34N4 = 726.28) |
| 146 | m/z = 672.77 | 148 | m/z = 802.98 |
| | (C47H33N2OP = 672.23) | | (C59H38N4 = 802.31) |
| 149 | m/z = 627.75 | 150 | m/z = 626.76 |
| | (C44H29N5 = 627.24) | | (C45H30N4 = 626.25) |
| 154 | m/z = 702.86 | 159 | m/z = 703.85 |
| | (C51H34N4 = 702.28) | | (C50H33N5 = 703.27) |
| 161 | m/z = 779.95 | 164 | m/z = 726.88 |
| | (C56H37N5 = 779.30) | | (C53H34N4 = 726.28) |
| 167 | m/z = 779.95 | 178 | m/z = 809.00 |
| | (C56H37N5 = 779.30) | | (C57H36N4S = 808.27) |
| 321 | m/z = 574.69 | 322 | m/z = 650.78 |
| | (C41H26N4 = 574.22) | | (C47H30N4 = 650.25) |
| 346 | m/z = 672.77 | 366 | m/z = 778.96 |
| | (C47H33N2OP = 672.23) | | (C57H38N4 = 778.31) |
| 371 | m/z = 792.95 | 389 | m/z = 726.88 |
| | (C56H36N6 = 792.30) | | (C53H34N4 = 726.28) |
| 407 | m/z = 722.83 | 414 | m/z = 702.86 |
| | (C51H35N2OP = 722.25) | | (C51H34N4 = 702.28) |
| 439 | m/z = 727.87 | 473 | m/z = 703.85 |
| | (C52H33N5 = 727.27) | | (C50H33N5 = 703.27) |
| 477 | m/z = 703.85 | 502 | m/z = 650.78 |
| | (C50H33N5 = 703.27) | | (C47H30N4 = 650.25) |

### Experimental Example

### Experimental Example 1

1) Manufacture of Organic Light-Emitting Device

A glass substrate coated with a thin film of ITO to a thickness of 1500 Å was washed with distilled water ultrasonic waves. After finishing the distilled water, it was ultrasonically washed with a solvent such as acetone, methanol, isopropyl alcohol, and the like, and dried, and then UVO treatment was performed for minutes using UV in a UV washer. Next, the substrate was transferred to a plasma cleaner (PT), and then plasma-treated for the ITO work function and residual film removal in a vacuum state and transferred to a thermal deposition equipment for organic deposition. An organic layer was formed in a single light-emitting stack structure on the ITO transparent electrode (anode). A hole injection layer was formed by depositing HAT-CN to a thickness of A, and then the hole transport layer NPD was doped with DNTPD within 10% and deposited to a thickness of 1500 Å, and TCTA was continuously deposited to a thickness of 200 Å. Next, a light-emitting layer including a t-Bu-perylene dopant was formed on the ADN host to a thickness of 250 Å. Next, the electron transport layer Alq₃ was formed to a thickness of 250 Å, an N-type charge-generating layer was formed to a thickness of 100 Å by doping the compounds described in Table 7 below with the alkali metal lithium, and the cathode Al was formed to a thickness of about 1,000 Å, thereby manufacturing an organic light-emitting device.

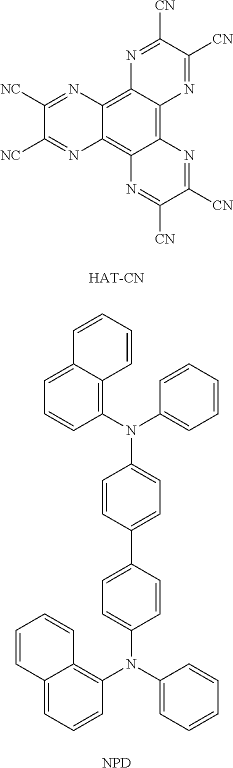

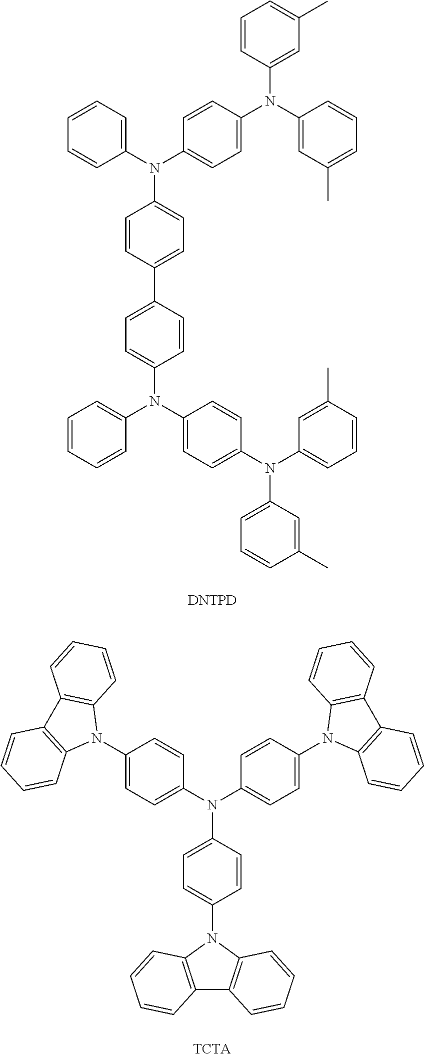 [SMILES: Cc1cccc(N(c2ccc(N(c3ccccc3)c3ccc(-c4ccc(N(c5ccccc5)c5ccc(N(c6cccc(C)c6)c6cccc(C)c6)cc5)cc4)cc3)cc2)c2cccc(C)c2)c1 ; "DNTPD" ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

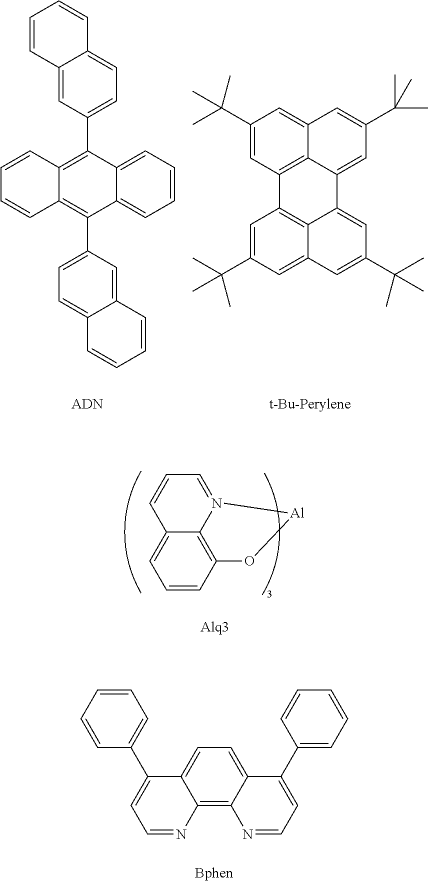

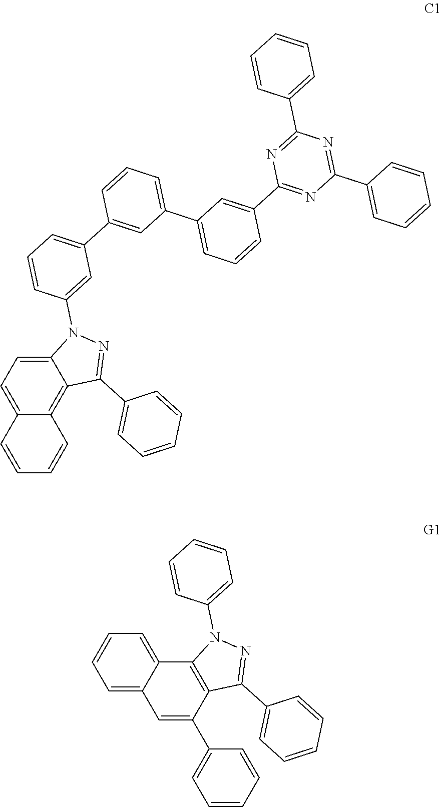 [SMILES: c1ccc(-c2nc(-c3ccccc3)nc(-c3cccc(-c4cccc(-c5cccc(-n6nc(-c7ccccc7)c7c8ccccc8ccc76)c5)c4)c3)n2)cc1 ; c1ccc(-c2cc3ccccc3c3c2c(-c2ccccc2)nn3-c2ccccc2)cc1]

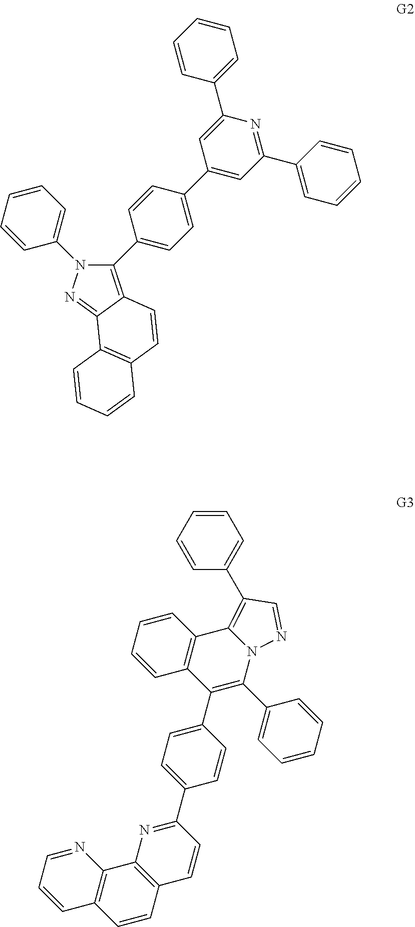 [SMILES: c1ccc(-c2cc(-c3ccc(-c4c5ccc6ccccc6c5nn4-c4ccccc4)cc3)cc(-c3ccccc3)n2)cc1 ; c1ccc(-c2cnn3c(-c4ccccc4)c(-c4ccc(-c5ccc6ccc7cccnc7c6n5)cc4)c4ccccc4c23)cc1]

2) Driving Voltage and Luminous Efficiency of Organic Light-Emitting Device

For the organic light-emitting device manufactured as described above, electroluminescence (EL) properties were measured with M7000 from McScience, and based on the measured results, T₉₅ was measured when the reference luminance was 750 cd/m² through the lifespan measuring device (M6000) manufactured by McScience. The measured results of the driving voltage, luminous efficiency, external quantum efficiency, and color coordinates (CIE) of the white organic light-emitting device manufactured according to the present invention are shown Table 7.

TABLE 7

| | | Driving | Luminous | | |
|---|---|---|---|---|---|
| | | Voltage | Efficiency | CIE | Lifespan |
| | Compound | (V) | (cd/A) | (x, y) | (T95) |
| Example | 1 | 5.05 | 6.71 | (0.134, | 33 |
| 1 | | | | 0.104) | |
| Example | 8 | 4.70 | 6.89 | (0.134, | 38 |
| 2 | | | | 0.104) | |
| Example | 9 | 4.88 | 6.56 | (0.134, | 31 |
| 3 | | | | 0.105) | |
| Example | 26 | 5.48 | 6.37 | (0.134, | 42 |
| 4 | | | | 0.103) | |
| Example | 28 | 5.50 | 6.57 | (0.134, | 33 |
| 5 | | | | 0.101) | |
| Example | 29 | 5.01 | 6.47 | (0.134, | 34 |
| 6 | | | | 0.101) | |
| Example | 30 | 5.66 | 6.71 | (0.133, | 32 |
| 7 | | | | 0.102) | |
| Example | 37 | 4.77 | 6.83 | (0.134, | 42 |
| 8 | | | | 0.100) | |
| Example | 38 | 5.00 | 6.34 | (0.133, | 32 |
| 9 | | | | 0.101) | |
| Example | 39 | 4.92 | 6.38 | (0.134, | 34 |
| 10 | | | | 0.101) | |
| Example | 46 | 4.89 | 6.90 | (0.134, | 38 |
| 11 | | | | 0.101) | |
| Example | 61 | 5.09 | 6.40 | (0.134, | 33 |
| 12 | | | | 0.101) | |
| Example | 68 | 5.39 | 6.87 | (0.133, | 34 |
| 13 | | | | 0.101) | |
| Example | 69 | 5.15 | 6.41 | (0.133, | 33 |
| 14 | | | | 0.102) | |
| Example | 79 | 5.34 | 6.44 | (0.134, | 32 |
| 15 | | | | 0.100) | |
| Example | 86 | 5.47 | 6.35 | (0.133, | 50 |
| 16 | | | | 0.101) | |
| Example | 87 | 5.26 | 6.33 | (0.134, | 52 |
| 17 | | | | 0.101) | |
| Example | 88 | 5.12 | 6.81 | (0.133, | 32 |
| 18 | | | | 0.100) | |
| Example | 93 | 5.03 | 6.33 | (0.134, | 32 |
| 19 | | | | 0.100) | |
| Example | 94 | 5.15 | 6.78 | (0.133, | 32 |
| 20 | | | | 0.101) | |
| Example | 98 | 5.11 | 6.45 | (0.134, | 33 |
| 21 | | | | 0.101) | |
| Example | 105 | 4.81 | 6.87 | (0.134, | 40 |
| 22 | | | | 0.099) | |
| Example | 112 | 4.99 | 6.67 | (0.134, | 31 |
| 23 | | | | 0.101) | |
| Example | 115 | 5.11 | 6.52 | (0.133, | 32 |
| 24 | | | | 0.101) | |
| Example | 117 | 5.17 | 6.49 | (0.134, | 33 |
| 25 | | | | 0.102) | |
| Example | 120 | 5.24 | 6.68 | (0.134, | 34 |
| 26 | | | | 0.101) | |
| Example | 121 | 5.22 | 6.81 | (0.132, | 32 |
| 27 | | | | 0.101) | |
| Example | 129 | 4.94 | 6.63 | (0.134, | 32 |
| 28 | | | | 0.103) | |
| Example | 146 | 5.61 | 6.42 | (0.134, | 49 |
| 29 | | | | 0.101) | |
| Example | 148 | 4.93 | 6.69 | (0.133, | 31 |
| 30 | | | | 0.102) | |
| Example | 149 | 5.00 | 6.69 | (0.133, | 33 |
| 31 | | | | 0.101) | |
| Example | 150 | 5.38 | 6.74 | (0.134, | 34 |
| 32 | | | | 0.100) | |
| Example | 154 | 4.83 | 6.64 | (0.134, | 31 |
| 33 | | | | 0.101) | |
| Example | 159 | 4.78 | 6.88 | (0.133, | 39 |
| 34 | | | | 0.100) | |
| Example | 161 | 5.00 | 6.58 | (0.133, | 31 |
| 35 | | | | 0.101) | |
| Example | 164 | 4.89 | 6.61 | (0.133, | 32 |
| 36 | | | | 0.101) | |
| Example | 167 | 4.77 | 6.91 | (0.133, | 36 |
| 37 | | | | 0.101) | |
| Example | 178 | 4.97 | 6.51 | (0.133, | 32 |
| 38 | | | | 0.099) | |
| Example | 321 | 5.51 | 6.38 | (0.134, | 33 |
| 39 | | | | 0.101) | |
| Example | 322 | 5.08 | 6.52 | (0.134, | 31 |
| 40 | | | | 0.100) | |
| Example | 346 | 5.69 | 6.44 | (0.134, | 53 |
| 41 | | | | 0.101) | |
| Example | 366 | 4.93 | 6.81 | (0.133, | 31 |
| 42 | | | | 0.101) | |
| Example | 371 | 5.03 | 6.57 | (0.134, | 31 |
| 43 | | | | 0.101) | |
| Example | 389 | 4.73 | 6.94 | (0.134, | 39 |
| 44 | | | | 0.101) | |
| Example | 407 | 5.66 | 6.39 | (0.133, | 48 |
| 45 | | | | 0.101) | |
| Example | 414 | 4.97 | 6.35 | (0.134, | 32 |
| 46 | | | | 0.101) | |
| Example | 439 | 5.02 | 6.42 | (0.134, | 32 |
| 47 | | | | 0.101) | |
| Example | 473 | 5.65 | 6.44 | (0.132, | 40 |
| 48 | | | | 0.101) | |
| Example | 477 | 5.54 | 6.38 | (0.133, | 33 |
| 49 | | | | 0.100) | |
| Example | 502 | 5.36 | 6.63 | (0.133, | 34 |
| 50 | | | | 0.100) | |
| Comparative | Bphen | 5.82 | 6.32 | (0.134, | 27 |
| Example | | | | 0.110) | |
| 1-1 | | | | | |
| Comparative | C1 | 5.78 | 6.22 | (0.134, | 29 |
| Example | | | | 0.111) | |
| 1-2 | | | | | |
| Comparative | G1 | 5.99 | 5.78 | (0.134, | 20 |
| Example | | | | 0.111) | |
| 1-3 | | | | | |
| Comparative | G2 | 5.81 | 6.12 | (0.133, | 28 |
| Example | | | | 0.102) | |
| 1-4 | | | | | |
| Comparative | G3 | 5.67 | 6.39 | (0.134, | 30 |
| Examples | | | | 0.108) | |
| 1-5 | | | | | |

From the results of Table 7 above, it was confirmed that the blue organic light-emitting device (single light-emitting stack structure) of the example using the compounds of the present invention as the charge-generating layer material had a lower driving voltage and improved luminous efficiency compared to the comparative examples. In particular, it was confirmed that the present invention provided remarkably superior effects in all aspects of driving voltage, efficiency, and lifespan.

Such results are presumed to be because the compounds of the present invention are composed of an appropriate heterocyclic compound having a skeleton with appropriate length, strength, and flat properties and capable of binding to a metal, thereby forming a gap state in the N-type charge-generating layer in a state doped with an alkali metal or alkaline earth metal. Specifically, it is presumed that excellent effects were exhibited because electrons generated from the P-type charge-generating layer were easily injected into the electron transport layer through the gap state generated in the N-type charge-generating layer.

That is, it is presumed that the P-type charge-generating layer is able to inject and transfer electrons well into the N-type charge-generating layer due to the above properties, thereby exhibiting a lowered driving voltage and improved luminous efficiency and lifespan properties of the organic light-emitting device.

Additionally, it was confirmed that the blue organic light-emitting device of Comparative Example 1-3 having an electron-generating layer composed of a compound having the same basic skeleton as the compounds of the present invention had poor driving voltage, efficiency, and lifespan properties when compared with other comparative examples (1-1 and 1-2). From such results, it can be seen that improved electroluminescence properties and lifespan properties cannot be obtained only with the basic skeleton of the compounds of the present invention. In addition, it can be confirmed that only when the basic skeleton is properly combined with various substituents as in the compounds of the present invention, proper physicochemical properties and thermal properties may be provided and excellent properties and results may be exhibited in device evaluation.

### Experimental Example 2

1) Manufacture of Organic Light-Emitting Device

A glass substrate coated with a thin film of ITO to a thickness of 1500 Å was washed with distilled water ultrasonic waves. After finishing the distilled water, it was ultrasonically washed with a solvent such as acetone, methanol, isopropyl alcohol, and the like, and dried, and then UVO treatment was performed for minutes using UV in a UV washer. Next, the substrate was transferred to a plasma cleaner (PT), and then plasma-treated for the ITO work function and residual film removal in a vacuum state and transferred to a thermal deposition equipment for organic deposition.

An organic layer was formed in a 2-light-emitting stack WOLED (white organic light-emitting device) structure on the ITO transparent electrode (anode).

In the case of a first light-emitting stack, a hole transport layer was formed by first thermally vacuum depositing TAPC to a thickness of 300 Å. After the hole transport layer was formed, a light-emitting layer was thermally vacuum deposited thereon as follows. The host TCz1 was doped with the blue phosphorescent dopant Flrpic at 8% to deposit the light-emitting layer at 300 Å. After an electron transport layer was formed at 400 Å using TmPyPB, a charge-generating layer was formed at 100 Å by doping the compounds described in Table 8 with Cs₂CO₃ at 20%.

In the case of a second light-emitting stack, a hole injection layer was formed by first thermally vacuum depositing MoO₃ to a thickness of 50 Å to form. A hole transport layer, which is a common layer, was formed by doping TAPC with MoO₃ at 20% to form 100 Å, and then depositing TAPC at 300 Å. A light-emitting layer was deposited at 300 Å thereon by doping the host TCz1 with the green phosphorescent dopant Ir(ppy)₃ at 8%, and then an electron transport layer was formed at 600 Å using TmPyPB. Finally, an electron injection layer was formed by depositing lithium fluoride (LiF) on the electron transport layer to a thickness of 10 Å, and then a cathode was formed by depositing an aluminum (Al) cathode to a thickness of 1,200 Å on the electron injection layer, thereby manufacturing a light-emitting device.

On the other hand, all organic compounds required for manufacturing OLED devices were vacuum sublimated and purified under 10⁻⁶ to 10⁻⁸ torr for each material before use in OLED manufacturing.

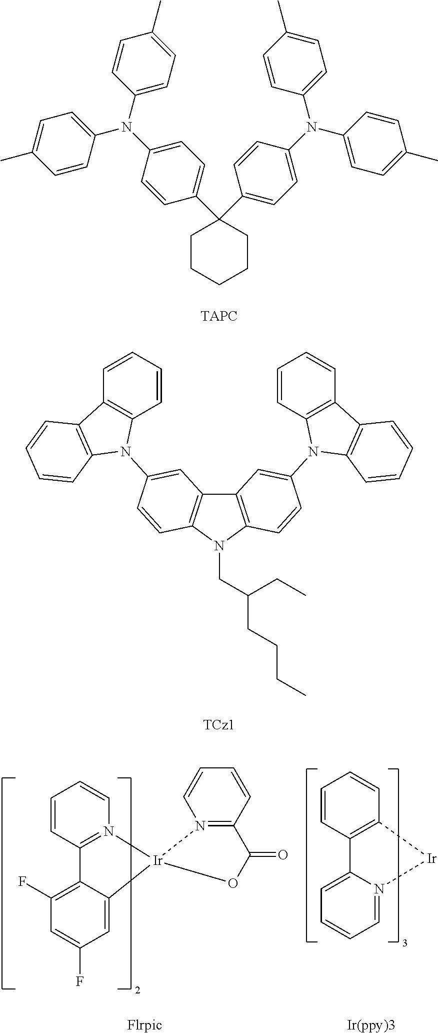

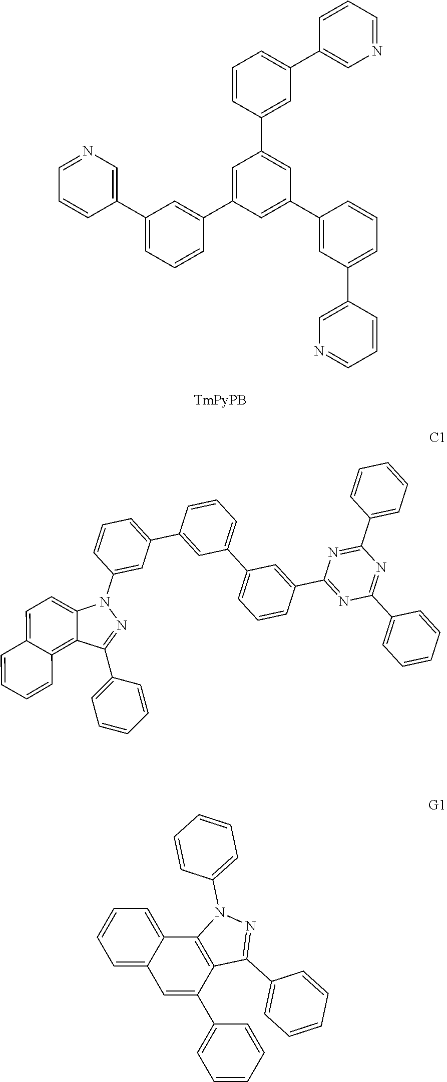 [SMILES: c1cncc(-c2cccc(-c3cc(-c4cccc(-c5cccnc5)c4)cc(-c4cccc(-c5cccnc5)c4)c3)c2)c1 ; c1ccc(-c2nc(-c3ccccc3)nc(-c3cccc(-c4cccc(-c5cccc(-n6nc(-c7ccccc7)c7c8ccccc8ccc76)c5)c4)c3)n2)cc1 ; c1ccc(-c2cc3ccccc3c3c2c(-c2ccccc2)nn3-c2ccccc2)cc1]

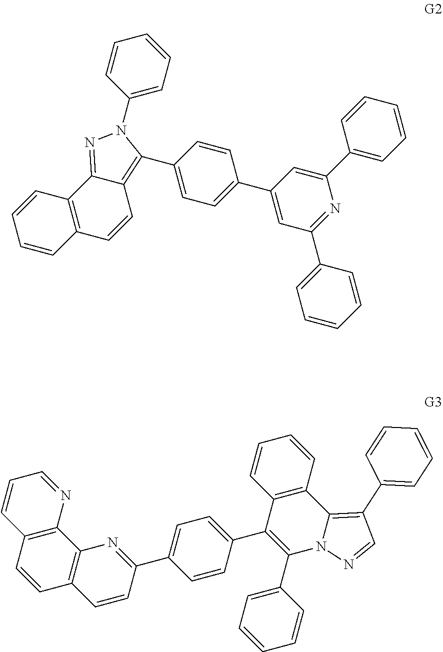 [SMILES: c1ccc(-c2cc(-c3ccc(-c4c5ccc6ccccc6c5nn4-c4ccccc4)cc3)cc(-c3ccccc3)n2)cc1 ; c1ccc(-c2cnn3c(-c4ccccc4)c(-c4ccc(-c5ccc6ccc7cccnc7c6n5)cc4)c4ccccc4c23)cc1]

2) Driving Voltage and Luminous Efficiency of Organic Light-Emitting Device

For the organic light-emitting device manufactured as described above, electroluminescence (EL) properties were measured with M7000 from McScience, and based on the measured results, T₉₅ was measured when the reference luminance was 3,500 cd/m² through the lifespan measuring device (M6000) manufactured by McScience. The measured results of the driving voltage, luminous efficiency, external quantum efficiency, and color coordinates (CIE) of the white organic light-emitting device manufactured according to the present invention are shown Table 8.

TABLE 8

| | | Driving | Luminous | | |
|---|---|---|---|---|---|
| | | Voltage | Efficiency | CIE | Lifespan |
| | Compound | (V) | (cd/A) | (x, y) | (T95) |
| Example | 1 | 7.91 | 60.95 | (0.218, | 31 |
| 51 | | | | 0.427) | |
| Example | 8 | 7.17 | 69.45 | (0.220, | 39 |
| 52 | | | | 0.431) | |
| Example | 9 | 7.45 | 68.88 | (0.200, | 29 |
| 53 | | | | 0.421) | |
| Example | 26 | 7.98 | 60.23 | (0.205, | 49 |
| 54 | | | | 0.411) | |
| Example | 28 | 7.93 | 63.21 | (0.221, | 32 |
| 55 | | | | 0.434) | |
| Example | 29 | 7.99 | 61.82 | (0.220, | 31 |
| 56 | | | | 0.440) | |
| Example | 30 | 7.49 | 68.98 | (0.219, | 29 |
| 57 | | | | 0.411) | |
| Example | 37 | 7.10 | 69.45 | (0.219, | 41 |
| 58 | | | | 0.429) | |
| Example | 38 | 8.01 | 64.22 | (0.215, | 33 |
| 59 | | | | 0.411) | |
| Example | 39 | 7.99 | 64.94 | (0.211, | 33 |
| 60 | | | | 0.419) | |
| Example | 46 | 7.21 | 68.26 | (0.209, | 35 |
| 61 | | | | 0.419) | |
| Example | 61 | 7.98 | 63.11 | (0.207, | 32 |
| 62 | | | | 0.409) | |
| Example | 68 | 7.50 | 66.66 | (0.208, | 31 |
| 63 | | | | 0.415) | |
| Example | 69 | 7.86 | 62.03 | (0.208, | 33 |
| 64 | | | | 0.412) | |
| Example | 79 | 7.94 | 61.98 | (0.208, | 34 |
| 65 | | | | 0.411) | |
| Example | 86 | 8.06 | 58.77 | (0.208, | 50 |
| 66 | | | | 0.412) | |
| Example | 87 | 8.14 | 59.05 | (0.209, | 49 |
| 67 | | | | 0.412) | |
| Example | 88 | 7.67 | 63.01 | (0.207, | 30 |
| 68 | | | | 0.411) | |
| Example | 93 | 7.30 | 68.21 | (0.231, | 31 |
| 69 | | | | 0.440) | |
| Example | 94 | 7.71 | 66.27 | (0.232, | 30 |
| 70 | | | | 0.422) | |
| Example | 98 | 7.89 | 62.11 | (0.230, | 31 |
| 71 | | | | 0.420) | |
| Example | 105 | 7.02 | 69.78 | (0.223, | 41 |
| 72 | | | | 0.433) | |
| Example | 112 | 7.53 | 64.47 | (0.222, | 32 |
| 73 | | | | 0.435) | |
| Example | 115 | 7.87 | 62.84 | (0.218, | 31 |
| 74 | | | | 0.421) | |
| Example | 117 | 7.99 | 64.96 | (0.220, | 32 |
| 75 | | | | 0.421) | |
| Example | 120 | 8.21 | 59.56 | (0.224, | 31 |
| 76 | | | | 0.429) | |
| Example | 121 | 7.55 | 64.99 | (0.215, | 32 |
| 77 | | | | 0.422) | |
| Example | 129 | 8.20 | 63.11 | (0.214, | 33 |
| 78 | | | | 0.420) | |
| Example | 146 | 7.97 | 59.32 | (0.230, | 52 |
| 79 | | | | 0.439) | |
| Example | 148 | 7.66 | 65.97 | (0.208, | 30 |
| 80 | | | | 0.412) | |
| Example | 149 | 8.28 | 59.04 | (0.231, | 32 |
| 81 | | | | 0.418) | |
| Example | 150 | 7.93 | 63.01 | (0.208, | 33 |
| 82 | | | | 0.412) | |
| Example | 154 | 7.36 | 67.03 | (0.209, | 30 |
| 83 | | | | 0.411) | |
| Example | 159 | 7.03 | 68.37 | (0.208, | 37 |
| 84 | | | | 0.412) | |
| Example | 161 | 7.89 | 62.67 | (0.233, | 32 |
| 85 | | | | 0.419) | |
| Example | 164 | 7.51 | 68.58 | (0.208, | 31 |
| 86 | | | | 0.412) | |
| Example | 167 | 7.21 | 69.36 | (0.207, | 36 |
| 87 | | | | 0.417) | |
| Example | 178 | 8.00 | 62.03 | (0.220, | 32 |
| 88 | | | | 0.412) | |
| Example | 321 | 7.90 | 59.80 | (0.231, | 33 |
| 89 | | | | 0.423) | |
| Example | 322 | 8.15 | 63.10 | (0.238, | 31 |
| 90 | | | | 0.423) | |
| Example | 346 | 7.98 | 61.88 | (0.209, | 48 |
| 91 | | | | 0.419) | |
| Example | 366 | 7.59 | 66.18 | (0.210, | 32 |
| 92 | | | | 0.420) | |
| Example | 371 | 7.99 | 63.97 | (0.211, | 33 |
| 93 | | | | 0.421) | |
| Example | 389 | 7.22 | 66.45 | (0.212, | 43 |
| 94 | | | | 0.422) | |
| Example | 407 | 8.03 | 59.10 | (0.228, | 54 |
| 95 | | | | 0.418) | |
| Example | 414 | 7.84 | 64.03 | (0.227, | 31 |
| 96 | | | | 0.412) | |
| Example | 439 | 8.00 | 61.25 | (0.229, | 33 |
| 97 | | | | 0.423) | |
| Example | 473 | 7.97 | 59.67 | (0.230, | 53 |
| 98 | | | | 0.421) | |
| Example | 477 | 8.09 | 59.20 | (0.231, | 33 |
| 99 | | | | 0.419) | |
| Example | 502 | 8.24 | 63.11 | (0.230, | 31 |
| 100 | | | | 0.423) | |
| Comparative | TmPyPB | 8.57 | 57.61 | (0.212, | 24 |
| Examples | | | | 0.433) | |
| 2-1 | | | | | |
| Comparative | C1 | 8.43 | 58.11 | (0.220, | 27 |
| Example | | | | 0.429) | |
| 2-2 | | | | | |
| Comparative | G1 | 8.61 | 57.55 | (0.222, | 23 |
| Example | | | | 0.430) | |
| 2-3 | | | | | |
| Comparative | G2 | 8.51 | 58.01 | (0.220, | 26 |
| Example | | | | 0.430) | |
| 2-4 | | | | | |
| Comparative | G3 | 8.37 | 58.68 | (0.212, | 29 |
| Example | | | | 0.421) | |
| 2-5 | | | | | |

From the results of Table 8 above, it can be confirmed that the white organic light-emitting device (2-light-emitting stack structure) of the example using the compounds of the present invention as the charge-generating layer material have a lower driving voltage and improved luminous efficiency compared to the comparative examples. In particular, it was confirmed that the present invention provided remarkably superior effects in all aspects of driving voltage, efficiency, and lifespan.

Such results are presumed to be because the compounds of the present invention are composed of an appropriate heterocyclic compound having a skeleton with appropriate length, strength, and flat properties and capable of binding to a metal, thereby forming a gap state in the N-type charge-generating layer in a state doped with an alkali metal or alkaline earth metal. Specifically, it is presumed that excellent effects were exhibited because electrons generated from the P-type charge-generating layer were easily injected into the electron transport layer through the gap state generated in the N-type charge-generating layer.

That is, it is presumed that the P-type charge-generating layer is able to inject and transfer electrons well into the N-type charge-generating layer due to the above properties, thereby exhibiting a lowered driving voltage and improved luminous efficiency and lifespan of the organic light-emitting device.

Additionally, it was confirmed that the white organic light-emitting device of Comparative Example 2-3 having an electron-generating layer composed of a compound having the same basic skeleton as the compounds of the present invention had poor driving voltage, efficiency, and lifespan properties when compared with other comparative examples (2-1 and 2-2). From such results, it can be seen that improved electroluminescence properties and lifespan properties cannot be obtained only with the basic skeleton of the compounds of the present invention. In addition, it can be confirmed that only when the basic skeleton is properly combined with various substituents as in the compounds of the present invention, proper physicochemical properties and thermal properties may be provided and excellent properties and results may be exhibited in device evaluation.

### Experimental Example 3

1) Manufacture of Organic Light-Emitting Device

The transparent electrode ITO thin film obtained from the glass for OLED was subject to ultrasonic washing for each 5 minutes using trichloroethylene, acetone, ethanol, and distilled water sequentially, and then stored in isopropanol before use.

Next, the ITO substrate was installed in the substrate folder of the vacuum deposition equipment, and the following 4,4′,4″-tris(N,N-(2-naphthyl)-phenylamino)triphenylamine (2-TNATA) was placed in the cell in the vacuum deposition equipment.

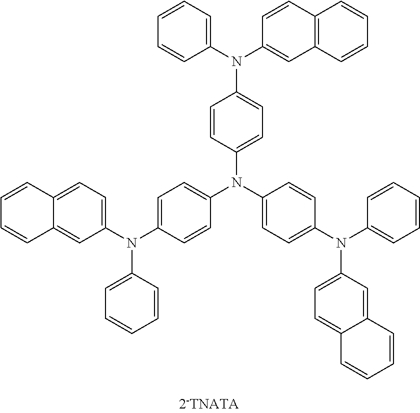 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Next, after evacuating the chamber until the vacuum degree reached 10⁻⁶ torr, an electric current was applied to the cell to evaporate 2-TNATA, thereby depositing a 600 Å-thick hole injection layer on the ITO substrate.

The following N,NY-bis(α-naphthyl)-N,NY-diphenyl-4,4′-diamine (NPB) was placed in another cell in the vacuum deposition equipment and evaporated by applying an electric current to the cell, thereby depositing a 300 Å-thick hole transport layer on the hole injection layer.

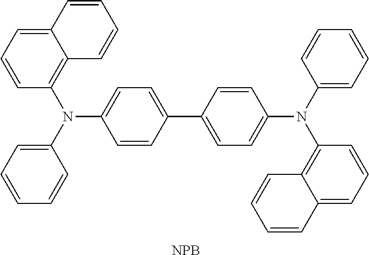 [SMILES: c1ccc(N(c2ccc(-c3ccc(N(c4ccccc4)c4cccc5ccccc45)cc3)cc2)c2cccc3ccccc23)cc1 ; "NPB"]

After the hole injection layer and the hole transport layer were formed in this way, a blue light-emitting material having the following structure was deposited as a light-emitting layer thereon. Specifically, the blue light-emitting host material H1 was vacuum-deposited to a thickness of 200 Å in one cell in the vacuum deposition equipment, and the blue light-emitting dopant material Dl was vacuum-deposited at 5% thereon compared to the host material.

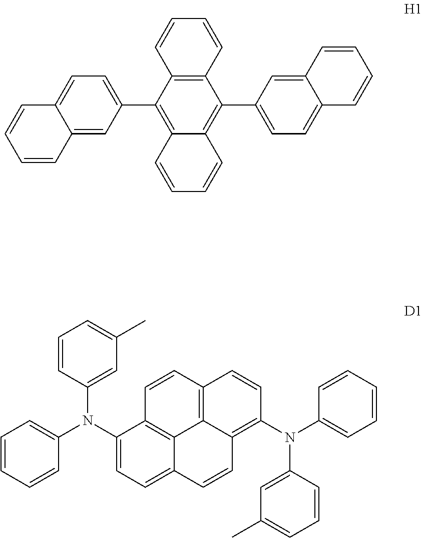

Next, an electron transport layer was deposited to a thickness of 300 Å with the compounds in Table 9 below.

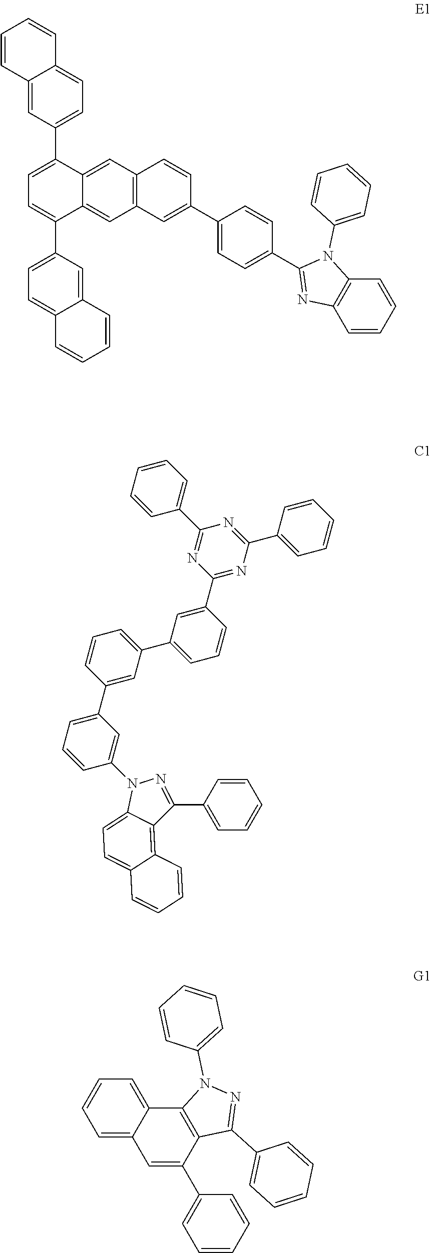 [SMILES: c1ccc(-n2c(-c3ccc(-c4ccc5cc6c(-c7ccc8ccccc8c7)ccc(-c7ccc8ccccc8c7)c6cc5c4)cc3)nc3ccccc32)cc1 ; c1ccc(-c2nc(-c3ccccc3)nc(-c3cccc(-c4cccc(-c5cccc(-n6nc(-c7ccccc7)c7c8ccccc8ccc76)c5)c4)c3)n2)cc1 ; c1ccc(-c2cc3ccccc3c3c2c(-c2ccccc2)nn3-c2ccccc2)cc1]

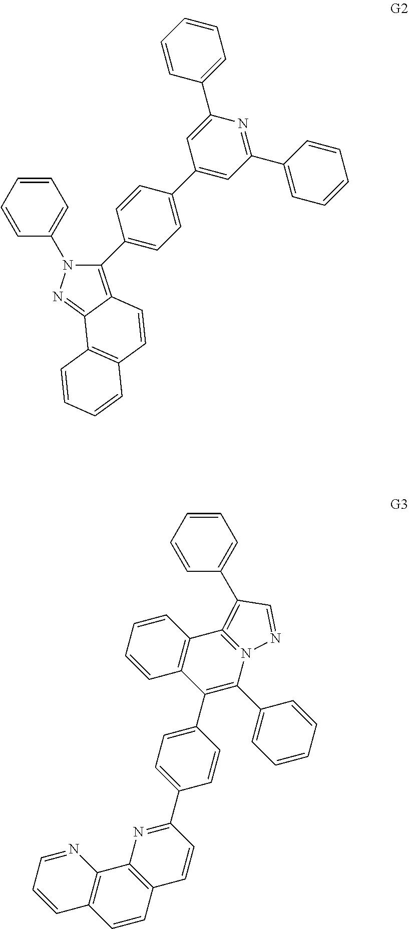

An electron injection layer was deposited to a thickness of A with lithium fluoride (LiF) and an Al cathode was deposited to a thickness of 1,000 Å, thereby manufacturing an OLED device.

On the other hand, all organic compounds required for manufacturing OLED devices were vacuum sublimated and purified under 10-6 to 10⁻⁸ torr for each material before use in OLED manufacturing.

2) Driving Voltage and Luminous Efficiency of Organic Light-Emitting Device

For the organic light-emitting device manufactured as described above, electroluminescence (EL) properties were measured with M7000 from McScience, and based on the measured results, T₉₅ was measured when the reference luminance was 700 cd/m² through the lifespan measuring device (M6000) manufactured by McScience. The measured results of the driving voltage, luminous efficiency, external quantum efficiency, and color coordinates (CIE) of the blue organic light-emitting device manufactured according to the present invention are shown Table 9.

TABLE 9

| | | Driving | Luminous | | |
|---|---|---|---|---|---|
| | | Voltage | Efficiency | CIE | Lifespan |
| | Compound | (V) | (cd/A) | (x, y) | (T95) |
| Example | 1 | 4.94 | 6.73 | (0.134, | 33 |
| 101 | | | | 0.101) | |
| Example | 8 | 4.71 | 6.90 | (0.134, | 40 |
| 102 | | | | 0.102) | |
| Example | 9 | 4.80 | 6.58 | (0.134, | 32 |
| 103 | | | | 0.101) | |
| Example | 26 | 5.38 | 6.18 | (0.134, | 50 |
| 104 | | | | 0.103) | |
| Example | 28 | 4.82 | 6.51 | (0.134, | 33 |
| 105 | | | | 0.102) | |
| Example | 29 | 5.23 | 6.22 | (0.134, | 32 |
| 106 | | | | 0.101) | |
| Example | 30 | 4.98 | 6.88 | (0.134, | 31 |
| 107 | | | | 0.102) | |
| Example | 37 | 4.77 | 6.98 | (0.134, | 38 |
| 108 | | | | 0.101) | |
| Example | 38 | 5.00 | 6.53 | (0.134, | 33 |
| 109 | | | | 0.101) | |
| Example | 39 | 5.14 | 6.50 | (0.134, | 32 |
| 110 | | | | 0.100) | |
| Example | 46 | 4.66 | 7.01 | (0.134, | 37 |
| 111 | | | | 0.101) | |
| Example | 61 | 5.01 | 6.41 | (0.134, | 33 |
| 112 | | | | 0.100) | |
| Example | 68 | 4.81 | 6.83 | (0.134, | 34 |
| 113 | | | | 0.100) | |
| Example | 69 | 4.91 | 6.55 | (0.134, | 36 |
| 114 | | | | 0.100) | |
| Example | 79 | 4.92 | 6.67 | (0.134, | 32 |
| 115 | | | | 0.100) | |
| Example | 86 | 5.03 | 6.38 | (0.134, | 55 |
| 116 | | | | 0.100) | |
| Example | 87 | 5.12 | 6.33 | (0.134, | 57 |
| 117 | | | | 0.102) | |
| Example | 88 | 4.71 | 6.99 | (0.134, | 33 |
| 118 | | | | 0.101) | |
| Example | 93 | 4.90 | 6.67 | (0.134, | 32 |
| 119 | | | | 0.102) | |
| Example | 94 | 4.98 | 6.62 | (0.134, | 33 |
| 120 | | | | 0.100) | |
| Example | 98 | 4.98 | 6.76 | (0.134, | 31 |
| 121 | | | | 0.103) | |
| Example | 105 | 4.68 | 6.85 | (0.134, | 38 |
| 122 | | | | 0.100) | |
| Example | 112 | 4.79 | 6.98 | (0.134, | 32 |
| 123 | | | | 0.102) | |
| Example | 115 | 5.42 | 6.26 | (0.134, | 32 |
| 124 | | | | 0.101) | |
| Example | 117 | 5.01 | 6.61 | (0.134, | 31 |
| 125 | | | | 0.100) | |
| Example | 120 | 5.05 | 6.77 | (0.134, | 32 |
| 126 | | | | 0.102) | |
| Example | 121 | 4.74 | 6.90 | (0.134, | 34 |
| 127 | | | | 0.103) | |
| Example | 129 | 4.99 | 6.69 | (0.134, | 31 |
| 128 | | | | 0.100) | |
| Example | 146 | 5.22 | 6.15 | (0.134, | 53 |
| 129 | | | | 0.103) | |
| Example | 148 | 4.79 | 6.68 | (0.134, | 32 |
| 130 | | | | 0.102) | |
| Example | 149 | 4.93 | 6.71 | (0.134, | 32 |
| 131 | | | | 0.100) | |
| Example | 150 | 4.98 | 6.75 | (0.134, | 32 |
| 132 | | | | 0.099) | |
| Example | 154 | 4.70 | 6.67 | (0.134, | 31 |
| 133 | | | | 0.102) | |
| Example | 159 | 4.75 | 6.93 | (0.134, | 37 |
| 134 | | | | 0.100) | |
| Example | 161 | 4.91 | 6.61 | (0.134, | 31 |
| 135 | | | | 0.103) | |
| Example | 164 | 5.01 | 6.53 | (0.134, | 35 |
| 136 | | | | 0.101) | |
| Example | 167 | 4.79 | 6.98 | (0.134, | 40 |
| 137 | | | | 0.104) | |
| Example | 178 | 5.03 | 6.50 | (0.134, | 32 |
| 138 | | | | 0.100) | |
| Example | 321 | 4.97 | 6.62 | (0.134, | 31 |
| 139 | | | | 0.103) | |
| Example | 322 | 4.91 | 6.55 | (0.134, | 32 |
| 140 | | | | 0.100) | |
| Example | 346 | 5.24 | 6.20 | (0.134, | 53 |
| 141 | | | | 0.102) | |
| Example | 366 | 4.81 | 6.91 | (0.134, | 34 |
| 142 | | | | 0.100) | |
| Example | 371 | 4.97 | 6.69 | (0.134, | 33 |
| 143 | | | | 0.101) | |
| Example | 389 | 4.67 | 6.85 | (0.134, | 37 |
| 144 | | | | 0.100) | |
| Example | 407 | 5.18 | 6.13 | (0.134, | 49 |
| 145 | | | | 0.101) | |
| Example | 414 | 5.00 | 6.11 | (0.134, | 33 |
| 146 | | | | 0.100) | |
| Example | 439 | 4.93 | 6.65 | (0.134, | 32 |
| 147 | | | | 0.101) | |
| Example | 473 | 5.05 | 6.23 | (0.134, | 55 |
| 148 | | | | 0.101) | |
| Example | 477 | 4.84 | 6.70 | (0.134, | 31 |
| 149 | | | | 0.100) | |
| Example | 502 | 5.43 | 6.17 | (0.134, | 34 |
| 150 | | | | 0.102) | |
| Comparative | E1 | 5.56 | 5.91 | (0.134, | 28 |
| Example | | | | 0.100) | |
| 3-1 | | | | | |
| Comparative | C1 | 5.50 | 6.10 | (0.134, | 30 |
| Example | | | | 0.101) | |
| 3-2 | | | | | |
| Comparative | G1 | 5.61 | 5.88 | (0.134, | 26 |
| Example | | | | 0.102) | |
| 3-3 | | | | | |
| Comparative | G2 | 5.46 | 6.08 | (0.134, | 28 |
| Example | | | | 0.101) | |
| 3-4 | | | | | |
| Comparative | G3 | 5.39 | 6.17 | (0.133, | 30 |
| Example | | | | 0.103) | |
| 3-5 | | | | | |

From the results of Table 9 above, it was confirmed that the blue organic light-emitting device of the examples using the compounds of the present invention as the electron transport layer material had a lower driving voltage and remarkably improved efficiency and lifespan compared to the comparative examples. In particular, it was confirmed that the examples using Compounds 8, 37, 46, 105, 159, 167, and 389 provided superior results in all aspects of driving voltage, efficiency, and lifespan.

Such results are judged to be due to the fact that when the compounds of the present invention having appropriate length, strength, and flat properties are used as the electron transport layer material, a compound in an excited state is made by accepting electrons under certain conditions, and in particular, if an excited state is formed at the heteroskeleton site of the compound, the excited heteroskeleton site returns to a stable state before reacting with other compounds, and thus, the stabilized compound is able to react with other compounds to efficiently transfer electrons without decomposition or destruction. For reference, compounds having a stable state when excited are aryls, acenes, or poly-membered heterocyclic compounds. Therefore, it is judged that the compounds of the present invention provide excellent effects in all aspects of driving voltage, efficiency, and lifespan due to improved electron transport properties or stability.

Additionally, it was confirmed that the blue organic light-emitting device of Comparative Example 3-3 having an electron transport layer composed of a compound having the same basic skeleton as the compounds of the present invention had poor driving voltage, luminous efficiency, and lifespan properties when compared with other comparative examples (3-1 and 3-2). From such results, it can be seen that improved electroluminescence properties and lifespan properties cannot be obtained only with the basic skeleton of the compounds of the present invention. In addition, it can be confirmed that only when the basic skeleton is properly combined with various substituents as in the compounds of the present invention, proper physicochemical properties and thermal properties may be provided and excellent properties and results may be exhibited in device evaluation.

The present invention is not limited to the above examples, but may be manufactured in a variety of different forms, and those of ordinary skill in the art to which the present invention pertains will understand that it may be implemented in other specific forms without changing the technical spirit or essential features of the present invention. Therefore, it should be understood that the examples described above are illustrative and not restrictive in all respects.

[Description of Symbols]

| | |
|---|---|
| 100: Substrate | 200: Anode |
| 300: Organic layer | 301: Hole injection layer |
| 302: Hole transport layer | 303: Light-emitting layer |
| 304: Hole-blocking layer | 305: Electron transport layer |
| 306: Electron injection layer | 400: Cathode |

1. A heterocyclic compound represented by following Formula 1: 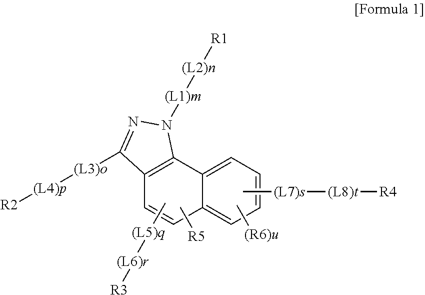
wherein:
R1 to R4 are the same as or different from each other and are each independently hydrogen, deuterium, halogen, a cyano group, a substituted or unsubstituted C1 to C60 alkyl group, a substituted or unsubstituted C2 to C60 alkenyl group, a substituted or unsubstituted C2 to C60 alkynyl group, a substituted or unsubstituted C1 to C60 alkoxy group, a substituted or unsubstituted C3 to C60 cycloalkyl group, a substituted or unsubstituted C2 to C60 heterocycloalkyl group, a substituted or unsubstituted C6 to C60 aryl group, a substituted or unsubstituted C2 to C60 heteroaryl group, or —P(═O)R101R102R103, wherein R101, R102, and R103 are the same as or different from each other and are each independently a substituted or unsubstituted C6 to C60 aryl group or a substituted or unsubstituted C2 to C60 heteroaryl group;
R5 and R6 are the same as or different from each other and are each independently hydrogen, deuterium, halogen, a cyano group, a substituted or unsubstituted C1 to C60 alkyl group, a substituted or unsubstituted C2 to C60 alkenyl group, a substituted or unsubstituted C2 to C60 alkynyl group, a substituted or unsubstituted C1 to C60 alkoxy group, a substituted or unsubstituted C3 to C60 cycloalkyl group, a substituted or unsubstituted C2 to C60 heterocycloalkyl group, a substituted or unsubstituted C6 to C60 aryl group, or a substituted or unsubstituted C2 to C60 heteroaryl group;
L1 to L8 are the same as or different from each other and are each independently a direct bond, a substituted or unsubstituted C6 to C60 arylene group, or a substituted or unsubstituted C2 to C60 heteroarylene group;
m, n, o, p, q, r, s, and t are the same as or different from each other and are each independently an integer of 0 to 2, provided that when m, n, o, p, q, r, s, and t are 2, each L1 to L8 defined by these are the same as or different from each other and are each independently selected; and
u is an integer of 0 to 4, provided that when u is 2 to 4, R6 is the same as or different from each other and is each independently selected.

2. The heterocyclic compound according to claim 1, wherein R1 to R3 are the same as or different from each other and are each independently a substituted or unsubstituted C6 to C60 aryl group, a substituted or unsubstituted C2 to C60 heteroaryl group, or —P(═O)R101R102R103, wherein R101, R102, and R103 are the same as or different from each other and are each independently a substituted or unsubstituted C6 to C60 aryl group or a substituted or unsubstituted C2 to C60 heteroaryl group.

3. The heterocyclic compound according to claim 2, wherein any one or more of -(L1)m-(L2)n-R1, -(L3)o-(L4)p-R2, -(L5)q-(L6)r-R3, and -(L7)s-(L8)t-R4 in Formula 1 comprise 2 to 8 aromatic rings with or without a heteroatom, wherein the 2 to 8 aromatic rings are composed of a monocyclic aromatic ring, an aromatic ring contained in a polycyclic condensed aromatic ring, or an aromatic ring included in a monocyclic aromatic ring and a polycyclic condensed aromatic ring.

4. The heterocyclic compound according to claim 3, wherein any one or more of -(L1)m-(L2)n-R1, -(L3)o-(L4)p-R2, -(L5)q-(L6)r-R3, and -(L7)s-(L8)t-R4 in Formula 1 comprise 3 to 8 aromatic rings with or without a heteroatom, wherein the 3 to 8 aromatic rings with or without a heteroatom are composed of a monocyclic aromatic ring, an aromatic ring contained in a polycyclic condensed aromatic ring, or an aromatic ring included in a monocyclic aromatic ring and a polycyclic condensed aromatic ring.

5. The heterocyclic compound according to claim 4, wherein the heterocyclic compound represented by Formula 1 is 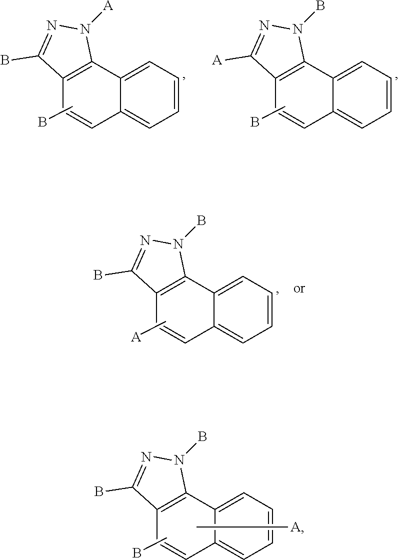
wherein:
A comprises 2 to 8 aromatic rings with or without a heteroatom, wherein the 2 to 8 aromatic rings are composed of a monocyclic aromatic ring, an aromatic ring contained in a polycyclic condensed aromatic ring, or an aromatic ring included in a monocyclic aromatic ring and a polycyclic condensed aromatic ring; and
B is a substituted or unsubstituted C6 to C30 aryl group or a substituted or unsubstituted C2 to C30 heteroaryl group.

6. The heterocyclic compound according to claim 5, wherein A comprises 5 to 7 aromatic rings with or without a heteroatom.

7. The heterocyclic compound according to claim 6, wherein A comprises a heteroatom.

8. The heterocyclic compound according to claim 7, wherein B is a substituted or unsubstituted phenyl group.

9. The heterocyclic compound according to claim 1, wherein the heterocyclic compound represented by Formula 1 is a compound represented by any one of the following compounds:

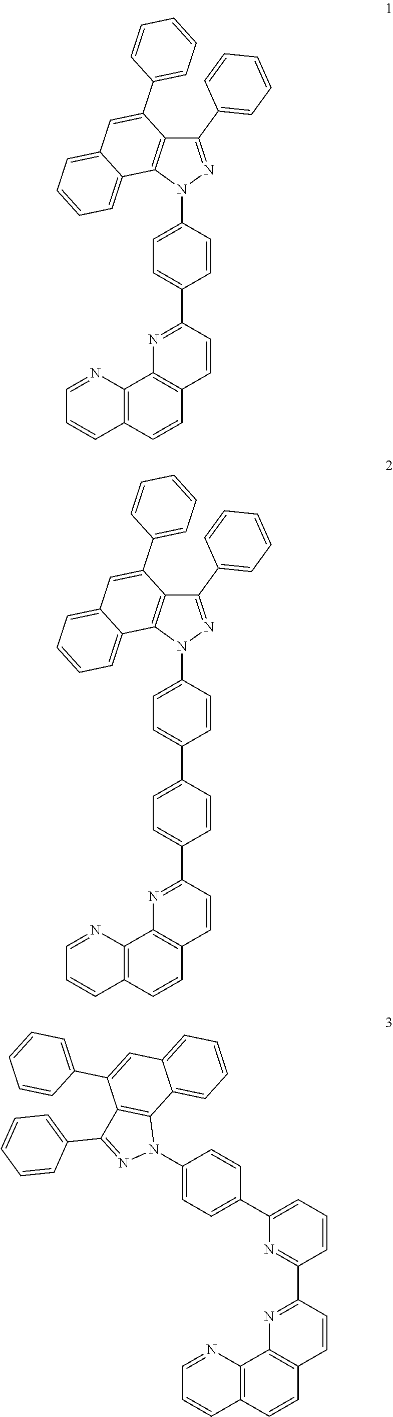

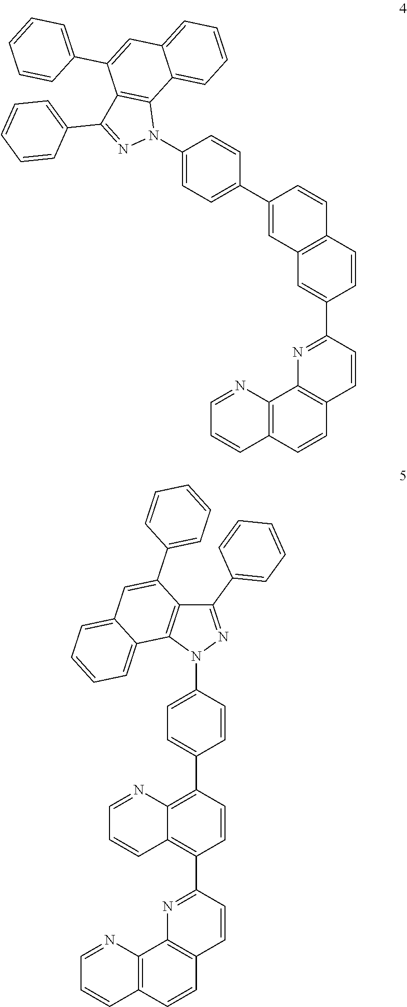 [SMILES: c1ccc(-c2cc3ccccc3c3c2c(-c2ccccc2)nn3-c2ccc(-c3ccc4ccc(-c5ccc6ccc7cccnc7c6n5)cc4c3)cc2)cc1 ; c1ccc(-c2cc3ccccc3c3c2c(-c2ccccc2)nn3-c2ccc(-c3ccc(-c4ccc5ccc6cccnc6c5n4)c4cccnc34)cc2)cc1]

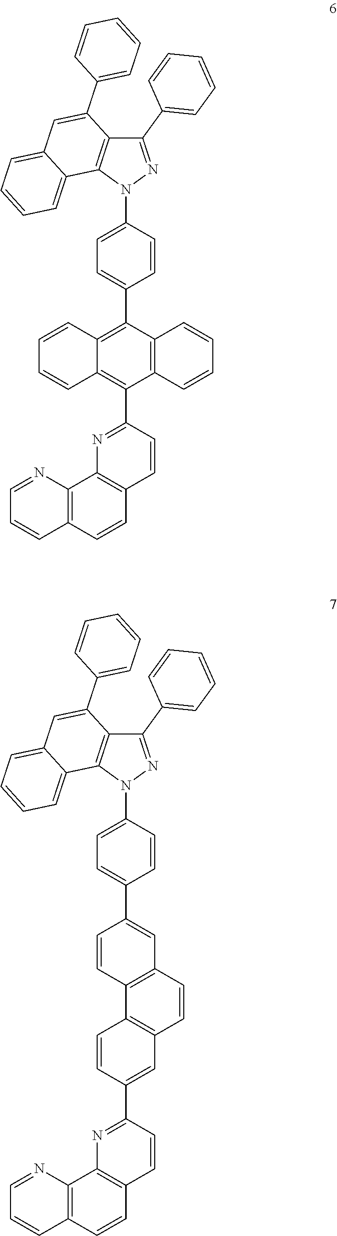 [SMILES: c1ccc(-c2cc3ccccc3c3c2c(-c2ccccc2)nn3-c2ccc(-c3c4ccccc4c(-c4ccc5ccc6cccnc6c5n4)c4ccccc34)cc2)cc1 ; c1ccc(-c2cc3ccccc3c3c2c(-c2ccccc2)nn3-c2ccc(-c3ccc4c(ccc5cc(-c6ccc7ccc8cccnc8c7n6)ccc54)c3)cc2)cc1]

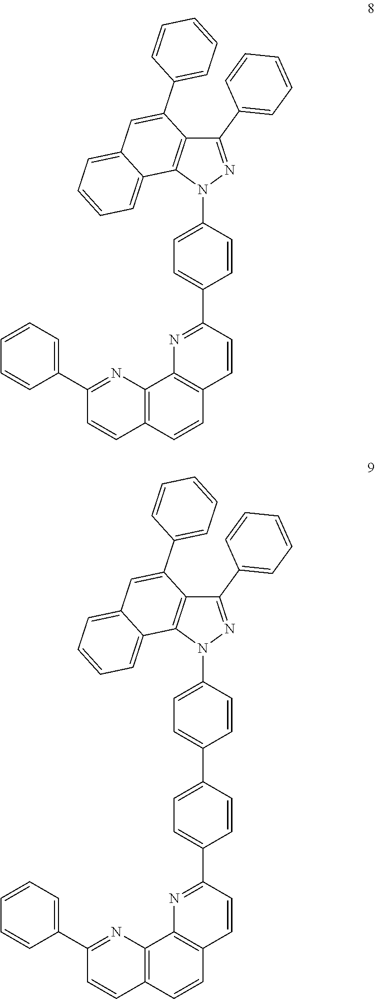

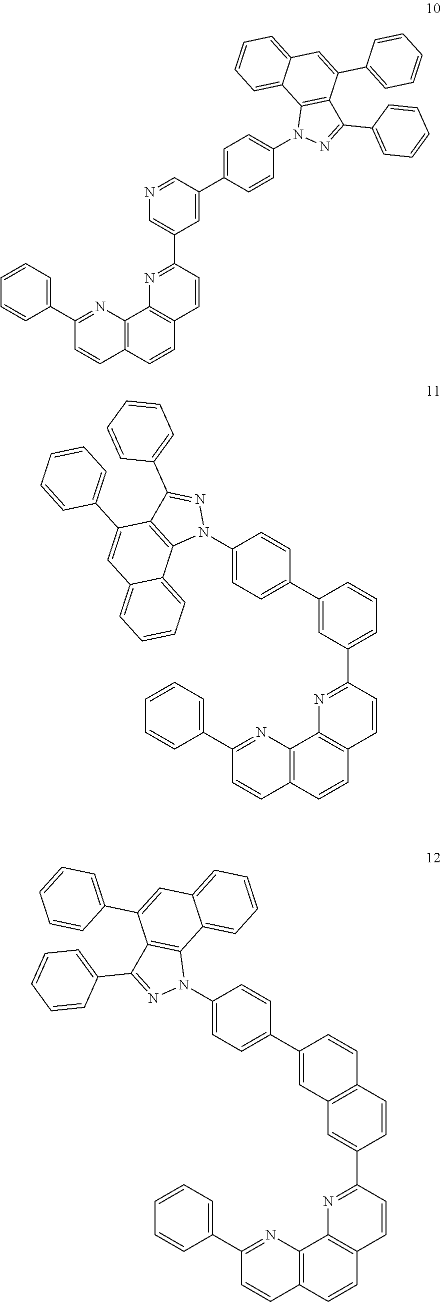

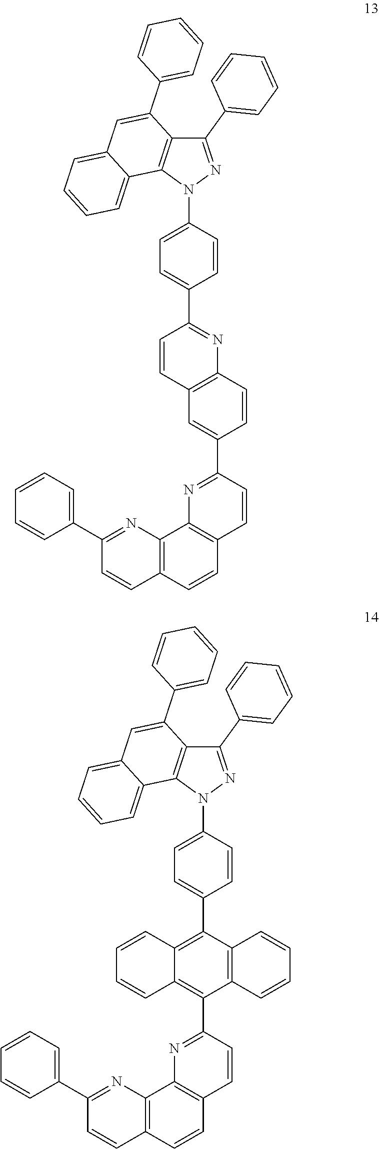

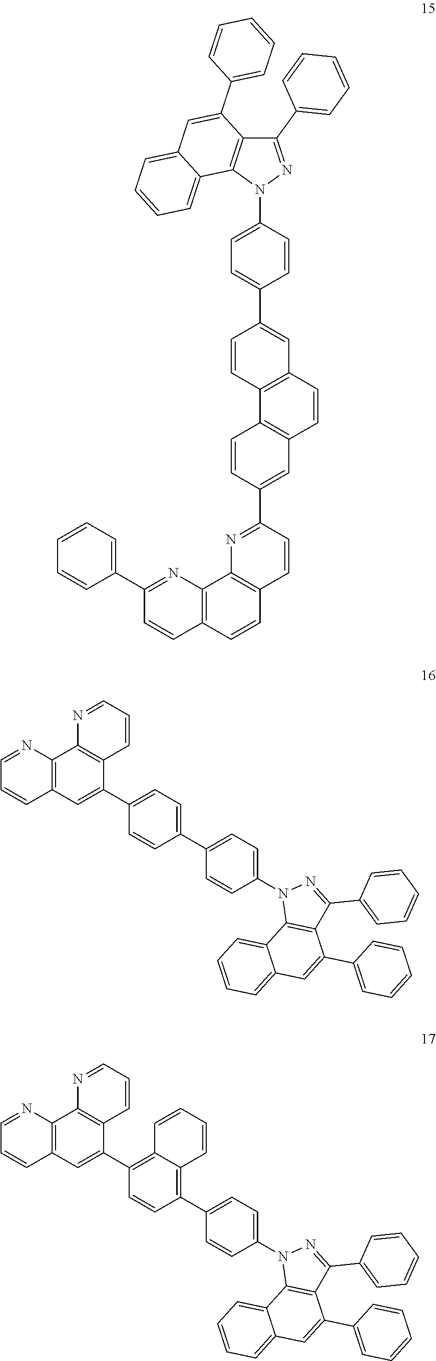 [SMILES: c1ccc(-c2ccc3ccc4ccc(-c5ccc6c(ccc7cc(-c8ccc(-n9nc(-c%10ccccc%10)c%10c(-c%11ccccc%11)cc%11ccccc%11c%109)cc8)ccc76)c5)nc4c3n2)cc1 ; c1ccc(-c2cc3ccccc3c3c2c(-c2ccccc2)nn3-c2ccc(-c3ccc(-c4cc5cccnc5c5ncccc45)cc3)cc2)cc1 ; c1ccc(-c2cc3ccccc3c3c2c(-c2ccccc2)nn3-c2ccc(-c3ccc(-c4cc5cccnc5c5ncccc45)c4ccccc34)cc2)cc1]

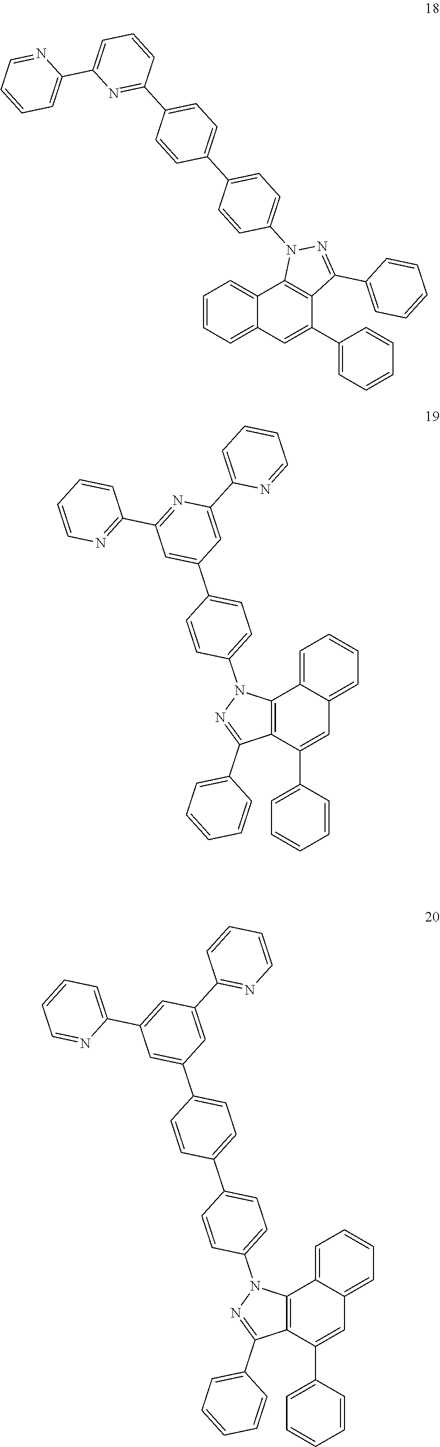 [SMILES: c1ccc(-c2cc3ccccc3c3c2c(-c2ccccc2)nn3-c2ccc(-c3ccc(-c4cccc(-c5ccccn5)n4)cc3)cc2)cc1 ; c1ccc(-c2cc3ccccc3c3c2c(-c2ccccc2)nn3-c2ccc(-c3cc(-c4ccccn4)nc(-c4ccccn4)c3)cc2)cc1 ; c1ccc(-c2cc3ccccc3c3c2c(-c2ccccc2)nn3-c2ccc(-c3ccc(-c4cc(-c5ccccn5)cc(-c5ccccn5)c4)cc3)cc2)cc1]

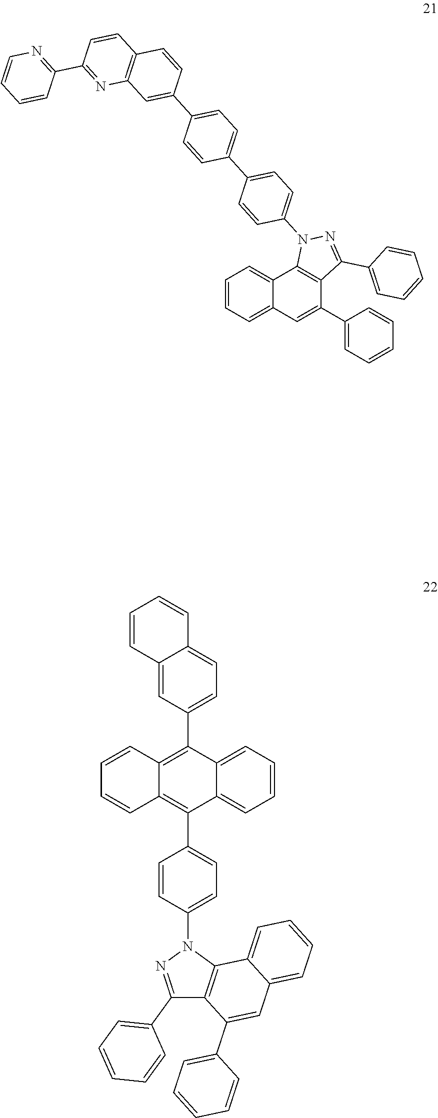 [SMILES: c1ccc(-c2cc3ccccc3c3c2c(-c2ccccc2)nn3-c2ccc(-c3ccc(-c4ccc5ccc(-c6ccccn6)nc5c4)cc3)cc2)cc1 ; c1ccc(-c2cc3ccccc3c3c2c(-c2ccccc2)nn3-c2ccc(-c3c4ccccc4c(-c4ccc5ccccc5c4)c4ccccc34)cc2)cc1]

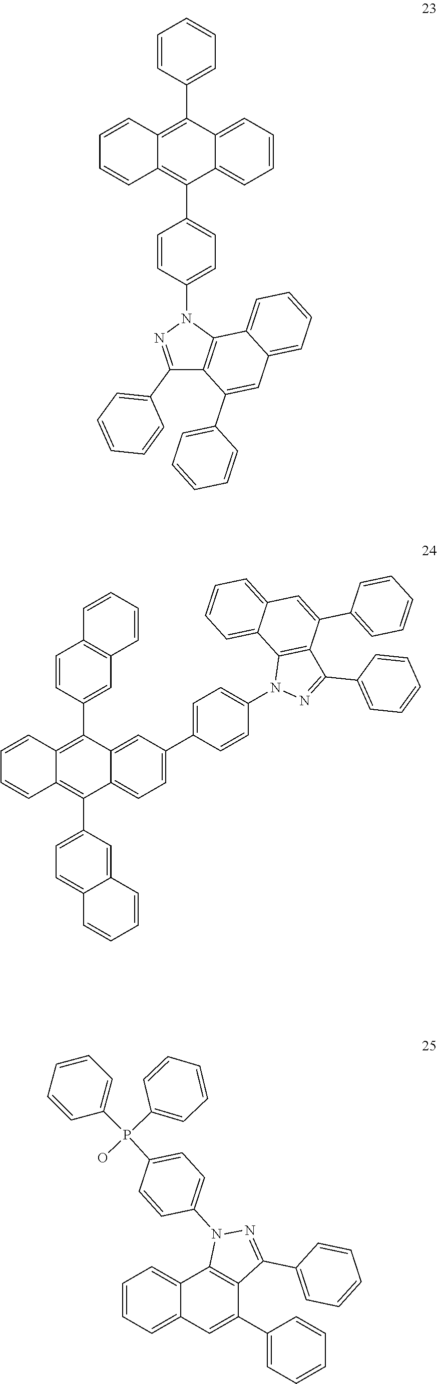

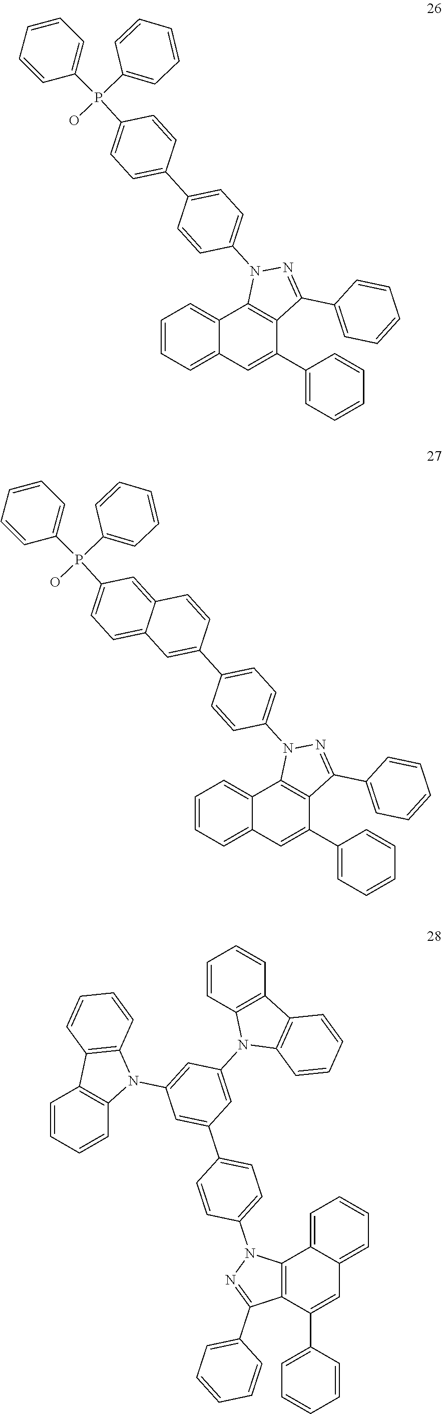

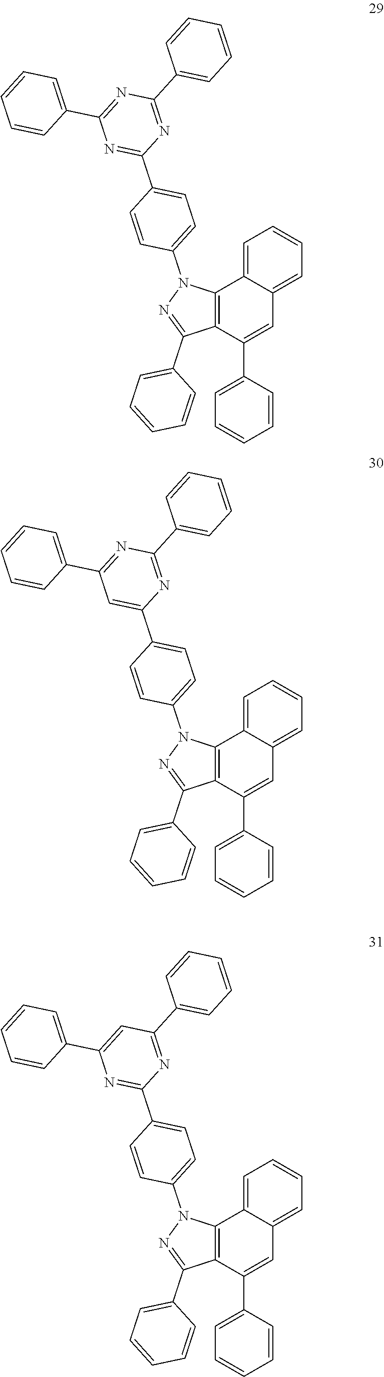

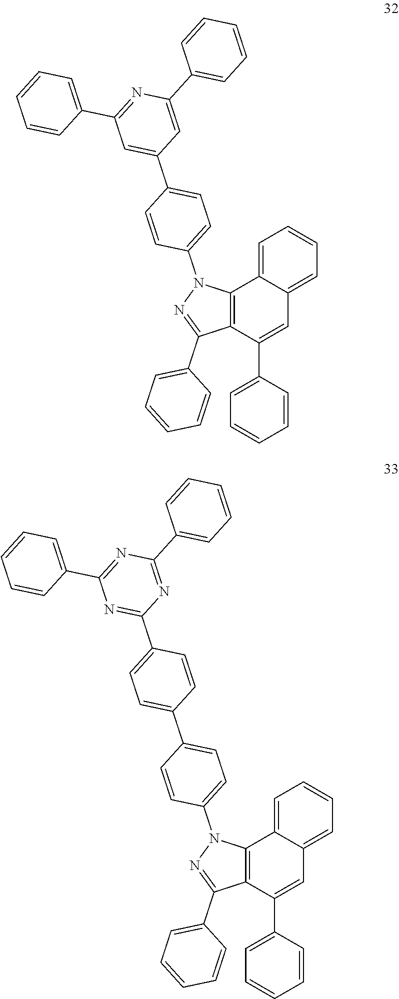 [SMILES: c1ccc(-c2cc(-c3ccc(-n4nc(-c5ccccc5)c5c(-c6ccccc6)cc6ccccc6c54)cc3)cc(-c3ccccc3)n2)cc1 ; c1ccc(-c2nc(-c3ccccc3)nc(-c3ccc(-c4ccc(-n5nc(-c6ccccc6)c6c(-c7ccccc7)cc7ccccc7c65)cc4)cc3)n2)cc1]

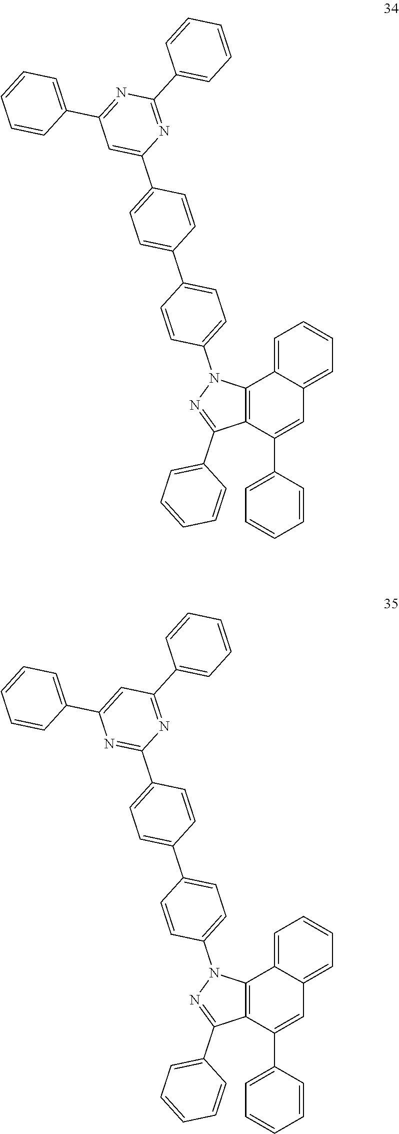 [SMILES: c1ccc(-c2cc(-c3ccc(-c4ccc(-n5nc(-c6ccccc6)c6c(-c7ccccc7)cc7ccccc7c65)cc4)cc3)nc(-c3ccccc3)n2)cc1 ; c1ccc(-c2cc(-c3ccccc3)nc(-c3ccc(-c4ccc(-n5nc(-c6ccccc6)c6c(-c7ccccc7)cc7ccccc7c65)cc4)cc3)n2)cc1]

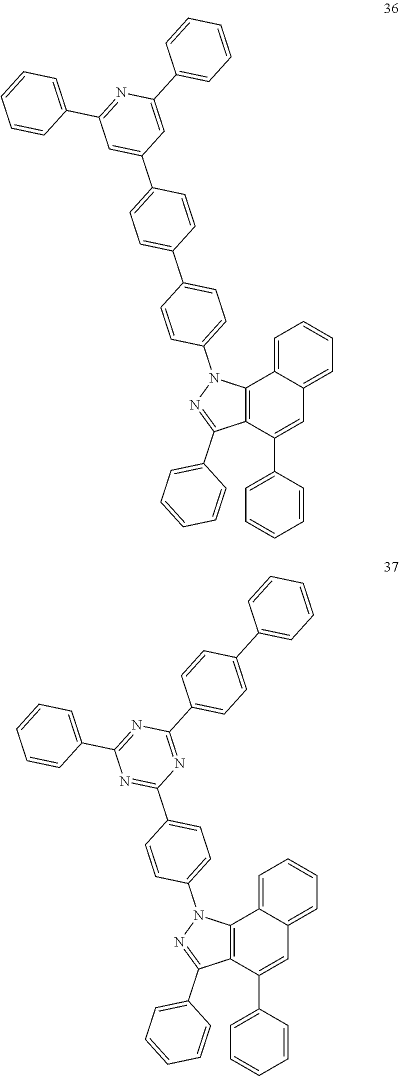

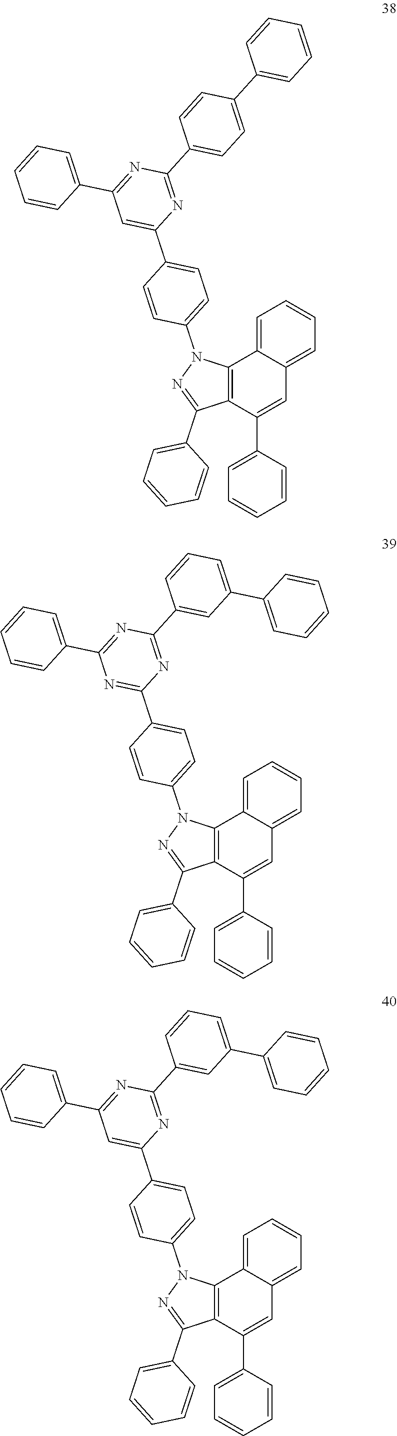

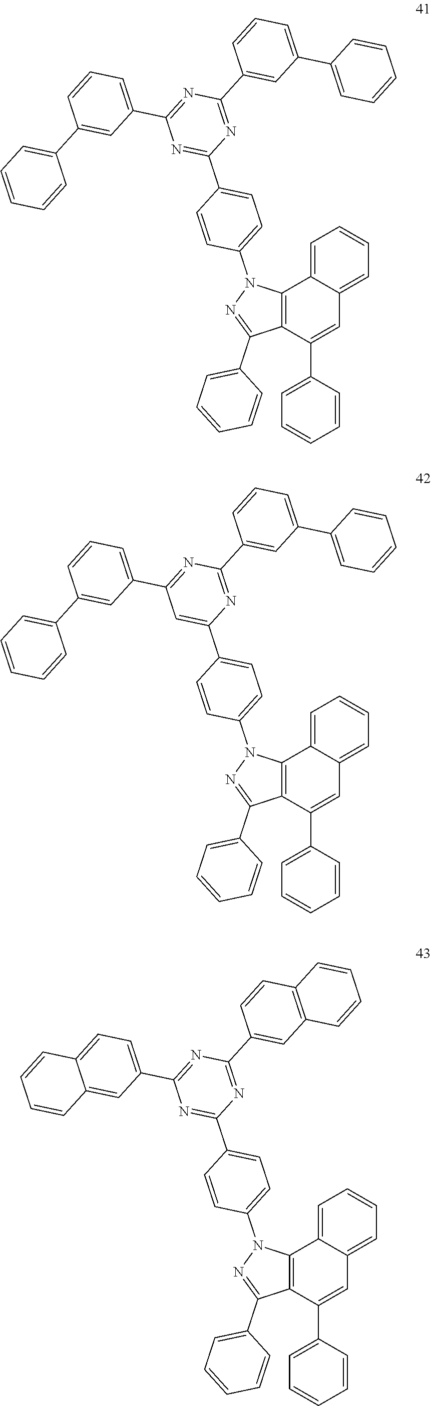 [SMILES: c1ccc(-c2cccc(-c3nc(-c4ccc(-n5nc(-c6ccccc6)c6c(-c7ccccc7)cc7ccccc7c65)cc4)nc(-c4cccc(-c5ccccc5)c4)n3)c2)cc1 ; c1ccc(-c2cccc(-c3cc(-c4ccc(-n5nc(-c6ccccc6)c6c(-c7ccccc7)cc7ccccc7c65)cc4)nc(-c4cccc(-c5ccccc5)c4)n3)c2)cc1 ; c1ccc(-c2cc3ccccc3c3c2c(-c2ccccc2)nn3-c2ccc(-c3nc(-c4ccc5ccccc5c4)nc(-c4ccc5ccccc5c4)n3)cc2)cc1]

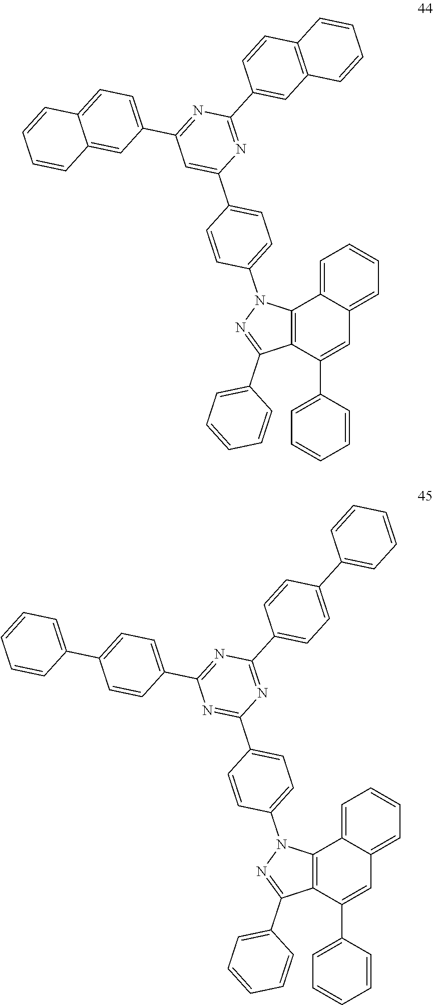 [SMILES: c1ccc(-c2cc3ccccc3c3c2c(-c2ccccc2)nn3-c2ccc(-c3cc(-c4ccc5ccccc5c4)nc(-c4ccc5ccccc5c4)n3)cc2)cc1 ; c1ccc(-c2ccc(-c3nc(-c4ccc(-c5ccccc5)cc4)nc(-c4ccc(-n5nc(-c6ccccc6)c6c(-c7ccccc7)cc7ccccc7c65)cc4)n3)cc2)cc1]

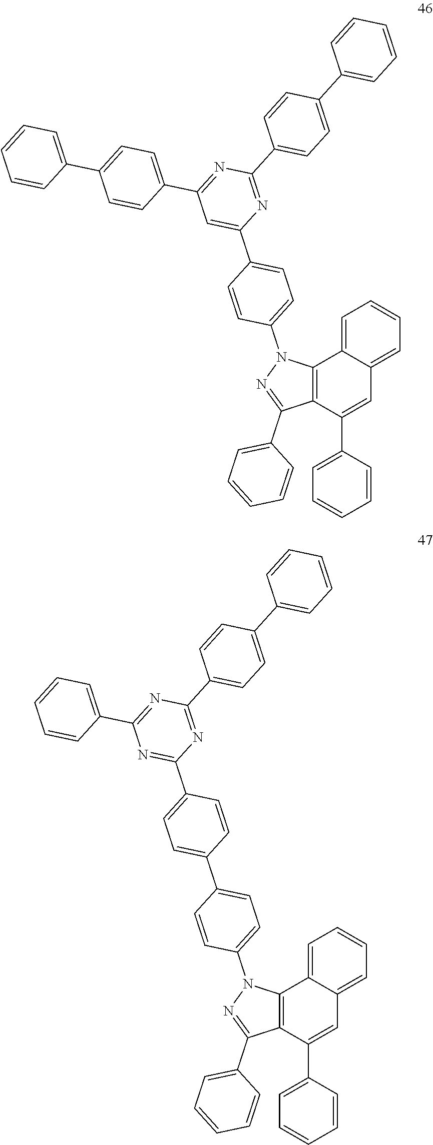

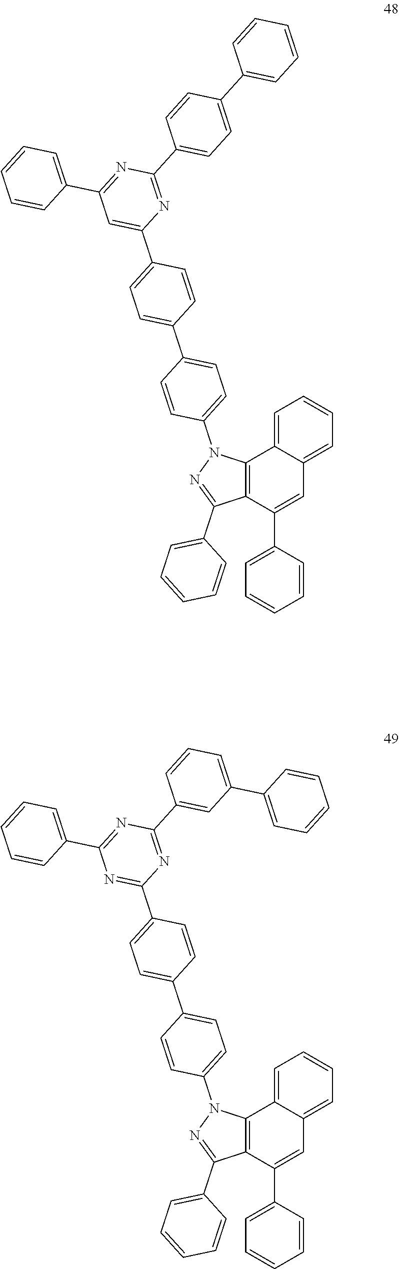 [SMILES: c1ccc(-c2ccc(-c3nc(-c4ccccc4)cc(-c4ccc(-c5ccc(-n6nc(-c7ccccc7)c7c(-c8ccccc8)cc8ccccc8c76)cc5)cc4)n3)cc2)cc1 ; c1ccc(-c2cccc(-c3nc(-c4ccccc4)nc(-c4ccc(-c5ccc(-n6nc(-c7ccccc7)c7c(-c8ccccc8)cc8ccccc8c76)cc5)cc4)n3)c2)cc1]

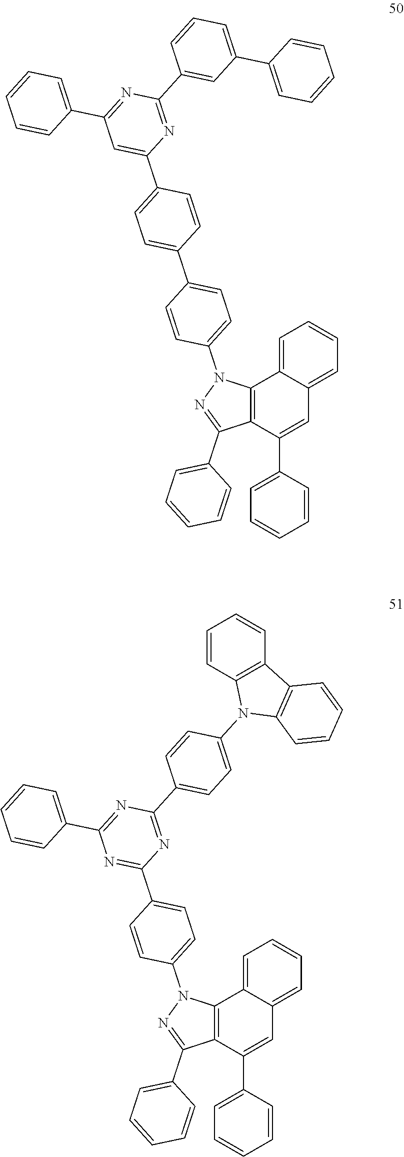 [SMILES: c1ccc(-c2cccc(-c3nc(-c4ccccc4)cc(-c4ccc(-c5ccc(-n6nc(-c7ccccc7)c7c(-c8ccccc8)cc8ccccc8c76)cc5)cc4)n3)c2)cc1 ; c1ccc(-c2nc(-c3ccc(-n4nc(-c5ccccc5)c5c(-c6ccccc6)cc6ccccc6c54)cc3)nc(-c3ccc(-n4c5ccccc5c5ccccc54)cc3)n2)cc1]

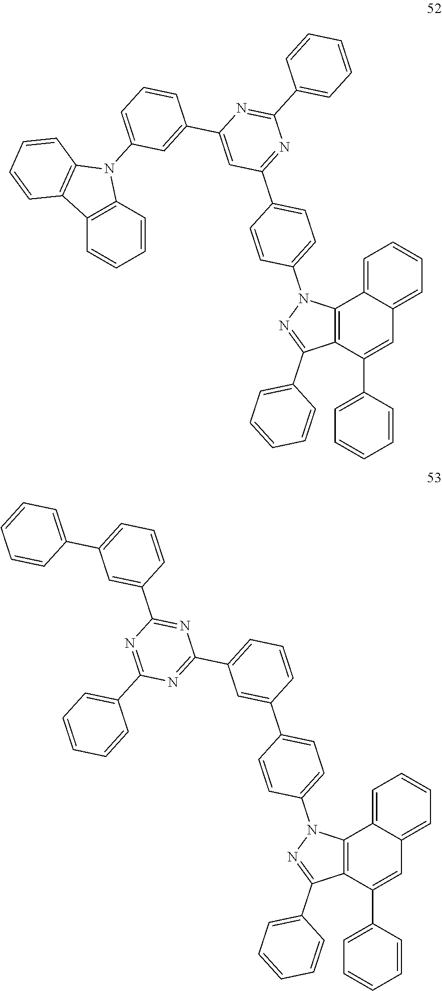 [SMILES: c1ccc(-c2nc(-c3ccc(-n4nc(-c5ccccc5)c5c(-c6ccccc6)cc6ccccc6c54)cc3)cc(-c3cccc(-n4c5ccccc5c5ccccc54)c3)n2)cc1 ; c1ccc(-c2cccc(-c3nc(-c4ccccc4)nc(-c4cccc(-c5ccc(-n6nc(-c7ccccc7)c7c(-c8ccccc8)cc8ccccc8c76)cc5)c4)n3)c2)cc1]

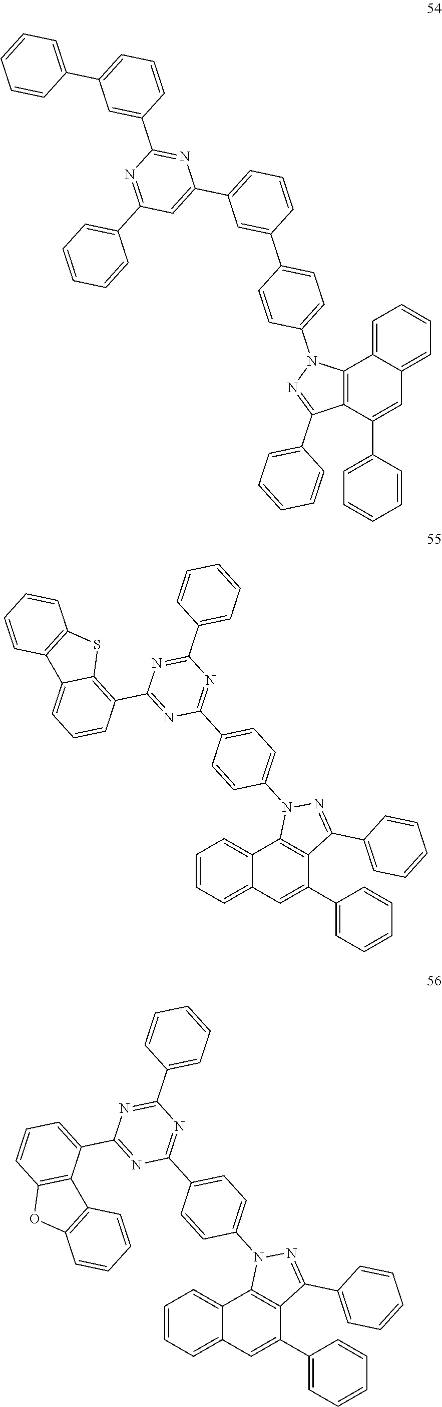 [SMILES: c1ccc(-c2cccc(-c3nc(-c4ccccc4)cc(-c4cccc(-c5ccc(-n6nc(-c7ccccc7)c7c(-c8ccccc8)cc8ccccc8c76)cc5)c4)n3)c2)cc1 ; c1ccc(-c2nc(-c3ccc(-n4nc(-c5ccccc5)c5c(-c6ccccc6)cc6ccccc6c54)cc3)nc(-c3cccc4c3sc3ccccc34)n2)cc1 ; c1ccc(-c2nc(-c3ccc(-n4nc(-c5ccccc5)c5c(-c6ccccc6)cc6ccccc6c54)cc3)nc(-c3cccc4oc5ccccc5c34)n2)cc1]

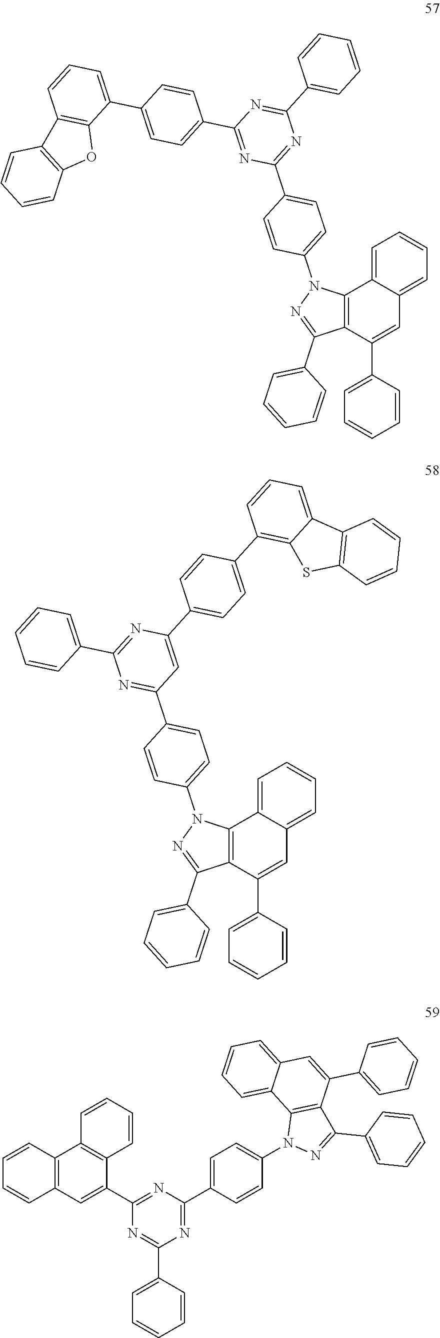 [SMILES: c1ccc(-c2nc(-c3ccc(-c4cccc5c4oc4ccccc45)cc3)nc(-c3ccc(-n4nc(-c5ccccc5)c5c(-c6ccccc6)cc6ccccc6c54)cc3)n2)cc1 ; c1ccc(-c2nc(-c3ccc(-c4cccc5c4sc4ccccc45)cc3)cc(-c3ccc(-n4nc(-c5ccccc5)c5c(-c6ccccc6)cc6ccccc6c54)cc3)n2)cc1 ; c1ccc(-c2nc(-c3ccc(-n4nc(-c5ccccc5)c5c(-c6ccccc6)cc6ccccc6c54)cc3)nc(-c3cc4ccccc4c4ccccc34)n2)cc1]

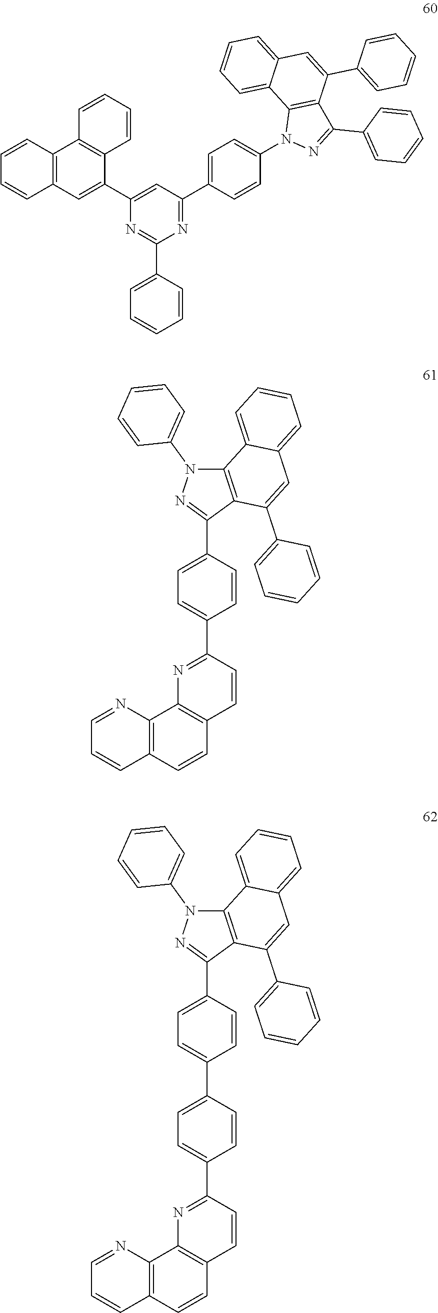 [SMILES: c1ccc(-c2nc(-c3ccc(-n4nc(-c5ccccc5)c5c(-c6ccccc6)cc6ccccc6c54)cc3)cc(-c3cc4ccccc4c4ccccc34)n2)cc1 ; c1ccc(-c2cc3ccccc3c3c2c(-c2ccc(-c4ccc5ccc6cccnc6c5n4)cc2)nn3-c2ccccc2)cc1 ; c1ccc(-c2cc3ccccc3c3c2c(-c2ccc(-c4ccc(-c5ccc6ccc7cccnc7c6n5)cc4)cc2)nn3-c2ccccc2)cc1]

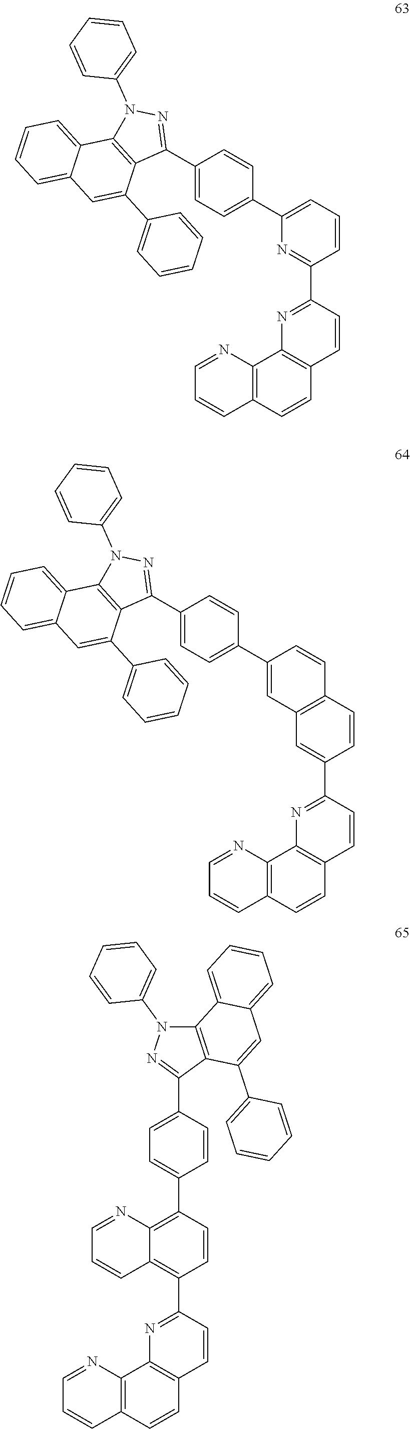

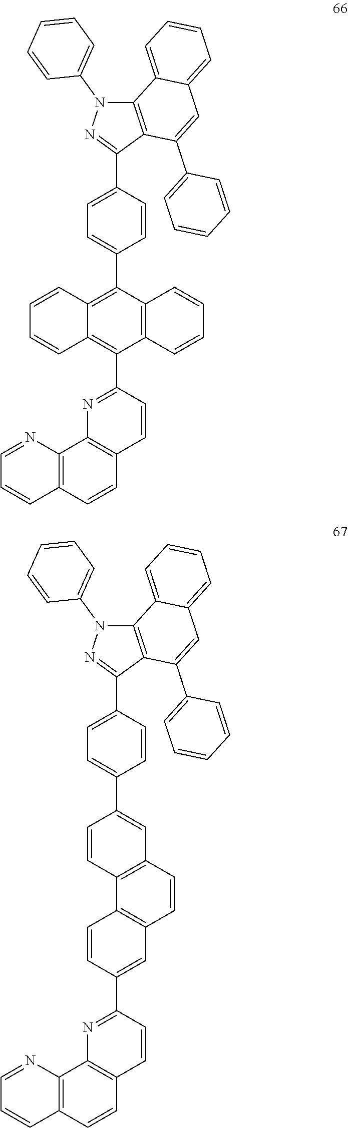

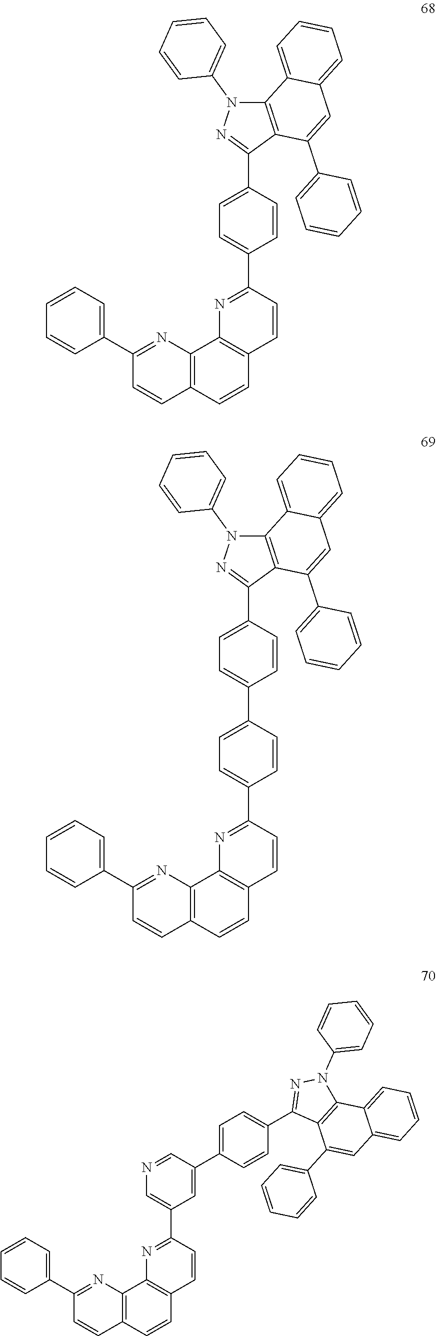 [SMILES: c1ccc(-c2ccc3ccc4ccc(-c5ccc(-c6nn(-c7ccccc7)c7c6c(-c6ccccc6)cc6ccccc67)cc5)nc4c3n2)cc1 ; c1ccc(-c2ccc3ccc4ccc(-c5ccc(-c6ccc(-c7nn(-c8ccccc8)c8c7c(-c7ccccc7)cc7ccccc78)cc6)cc5)nc4c3n2)cc1 ; c1ccc(-c2ccc3ccc4ccc(-c5cncc(-c6ccc(-c7nn(-c8ccccc8)c8c7c(-c7ccccc7)cc7ccccc78)cc6)c5)nc4c3n2)cc1]

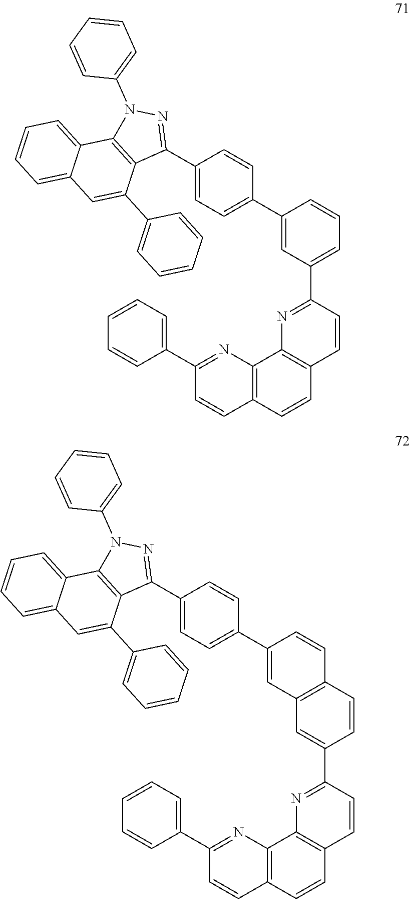 [SMILES: c1ccc(-c2ccc3ccc4ccc(-c5cccc(-c6ccc(-c7nn(-c8ccccc8)c8c7c(-c7ccccc7)cc7ccccc78)cc6)c5)nc4c3n2)cc1 ; c1ccc(-c2ccc3ccc4ccc(-c5ccc6ccc(-c7ccc(-c8nn(-c9ccccc9)c9c8c(-c8ccccc8)cc8ccccc89)cc7)cc6c5)nc4c3n2)cc1]

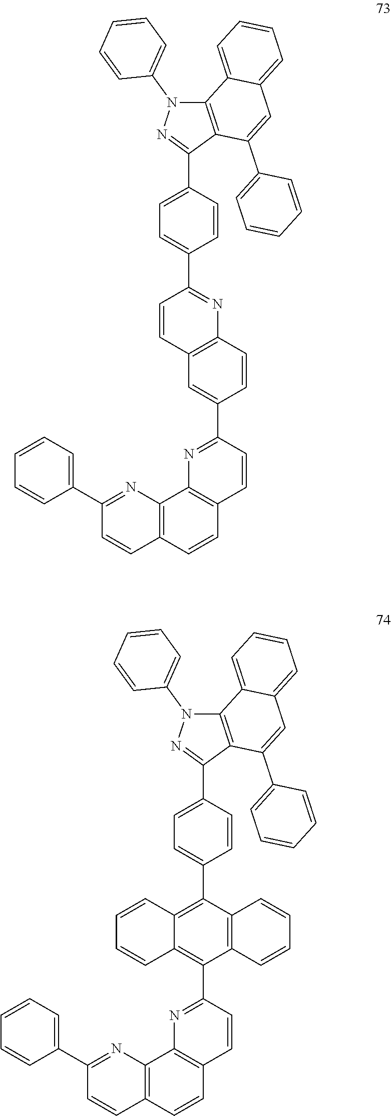 [SMILES: c1ccc(-c2ccc3ccc4ccc(-c5ccc6nc(-c7ccc(-c8nn(-c9ccccc9)c9c8c(-c8ccccc8)cc8ccccc89)cc7)ccc6c5)nc4c3n2)cc1 ; c1ccc(-c2ccc3ccc4ccc(-c5c6ccccc6c(-c6ccc(-c7nn(-c8ccccc8)c8c7c(-c7ccccc7)cc7ccccc78)cc6)c6ccccc56)nc4c3n2)cc1]

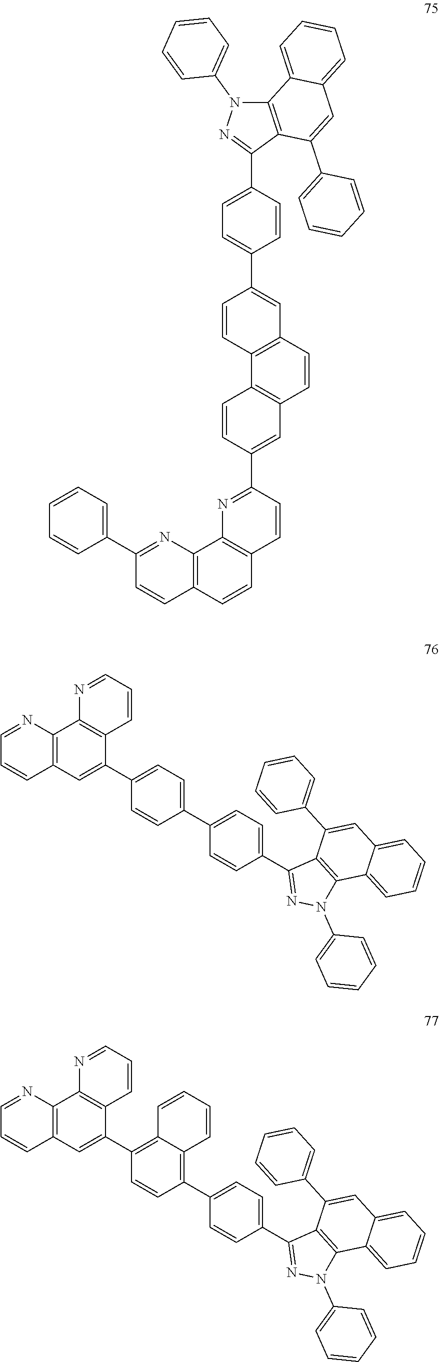 [SMILES: c1ccc(-c2ccc3ccc4ccc(-c5ccc6c(ccc7cc(-c8ccc(-c9nn(-c%10ccccc%10)c%10c9c(-c9ccccc9)cc9ccccc9%10)cc8)ccc76)c5)nc4c3n2)cc1 ; c1ccc(-c2cc3ccccc3c3c2c(-c2ccc(-c4ccc(-c5cc6cccnc6c6ncccc56)cc4)cc2)nn3-c2ccccc2)cc1 ; c1ccc(-c2cc3ccccc3c3c2c(-c2ccc(-c4ccc(-c5cc6cccnc6c6ncccc56)c5ccccc45)cc2)nn3-c2ccccc2)cc1]

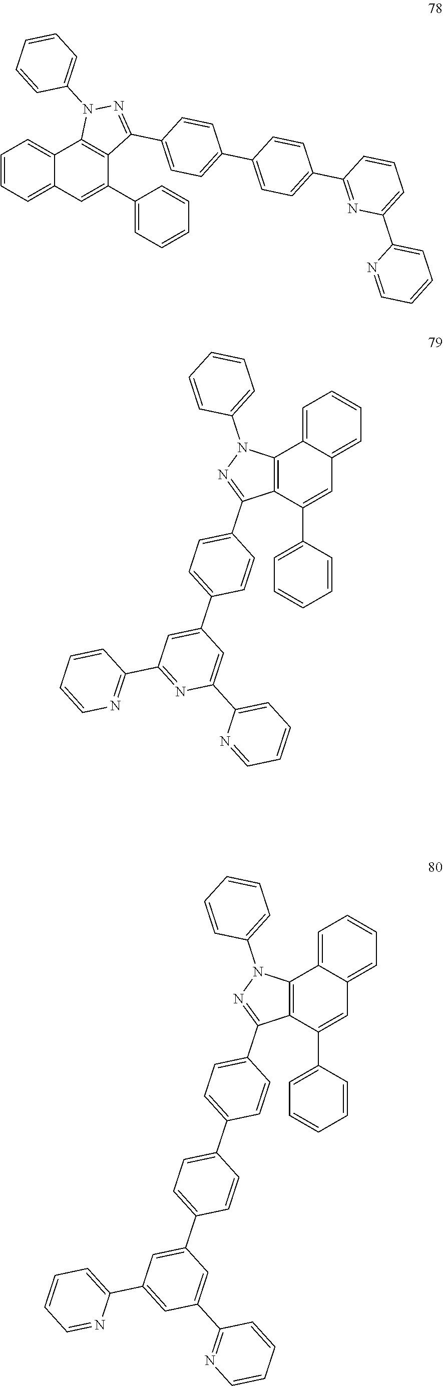 [SMILES: c1ccc(-c2cc3ccccc3c3c2c(-c2ccc(-c4ccc(-c5cccc(-c6ccccn6)n5)cc4)cc2)nn3-c2ccccc2)cc1 ; c1ccc(-c2cc3ccccc3c3c2c(-c2ccc(-c4cc(-c5ccccn5)nc(-c5ccccn5)c4)cc2)nn3-c2ccccc2)cc1 ; c1ccc(-c2cc3ccccc3c3c2c(-c2ccc(-c4ccc(-c5cc(-c6ccccn6)cc(-c6ccccn6)c5)cc4)cc2)nn3-c2ccccc2)cc1]

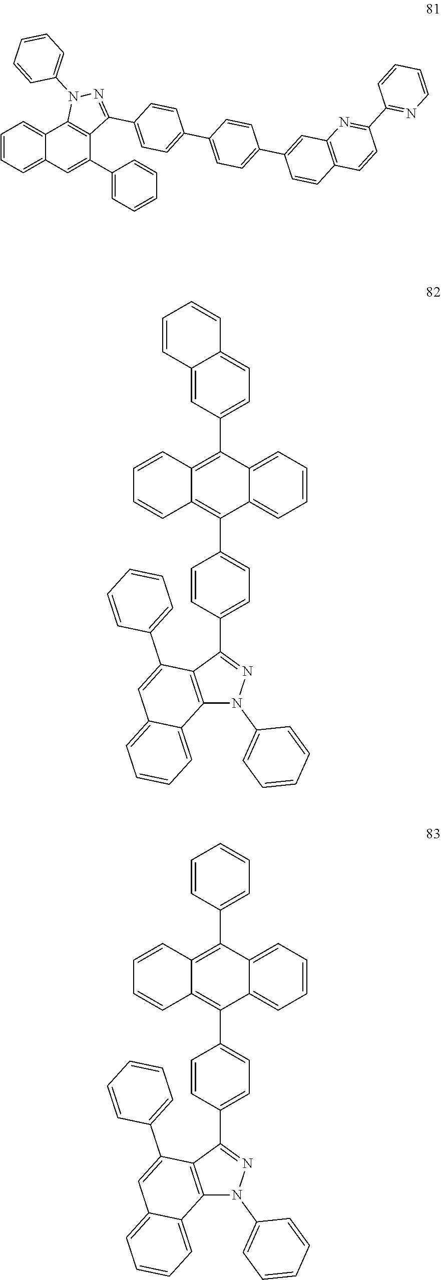 [SMILES: c1ccc(-c2cc3ccccc3c3c2c(-c2ccc(-c4ccc(-c5ccc6ccc(-c7ccccn7)nc6c5)cc4)cc2)nn3-c2ccccc2)cc1 ; c1ccc(-c2cc3ccccc3c3c2c(-c2ccc(-c4c5ccccc5c(-c5ccc6ccccc6c5)c5ccccc45)cc2)nn3-c2ccccc2)cc1 ; c1ccc(-c2c3ccccc3c(-c3ccc(-c4nn(-c5ccccc5)c5c4c(-c4ccccc4)cc4ccccc45)cc3)c3ccccc23)cc1]

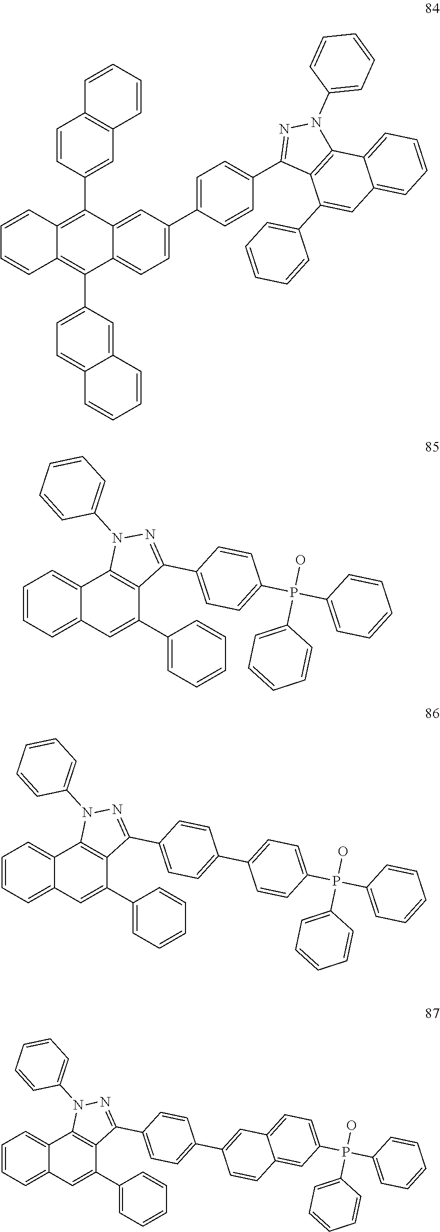

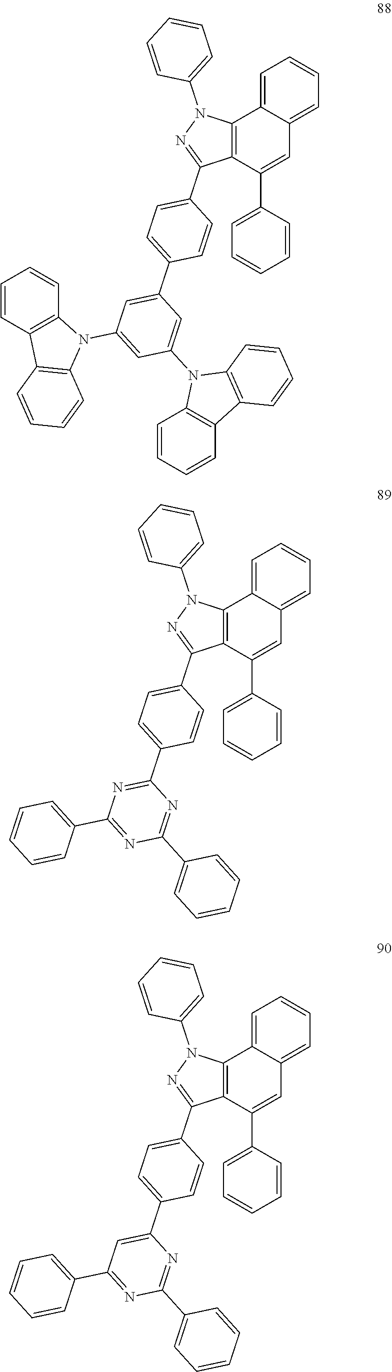

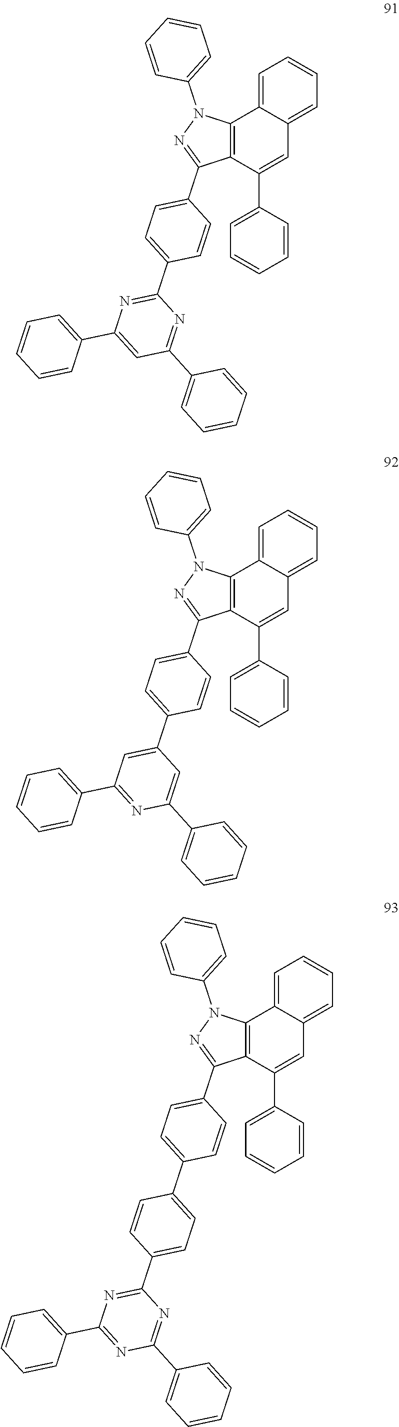

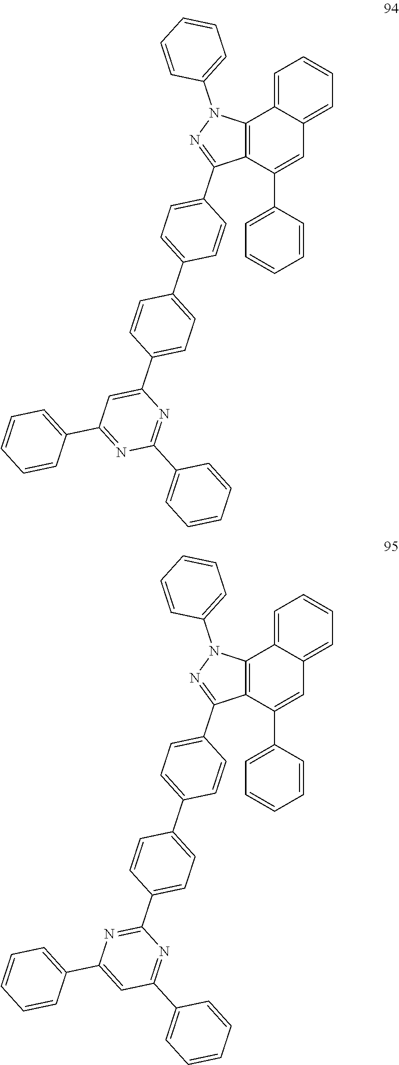

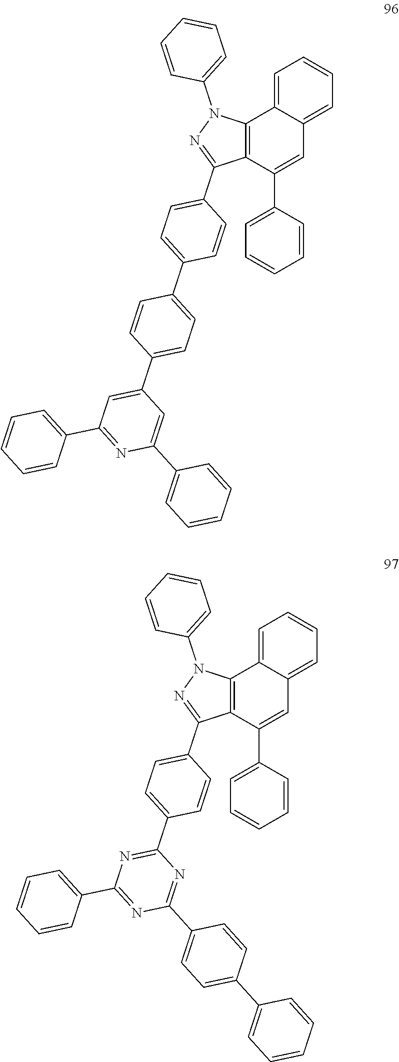 [SMILES: c1ccc(-c2cc(-c3ccc(-c4ccc(-c5nn(-c6ccccc6)c6c5c(-c5ccccc5)cc5ccccc56)cc4)cc3)cc(-c3ccccc3)n2)cc1 ; c1ccc(-c2ccc(-c3nc(-c4ccccc4)nc(-c4ccc(-c5nn(-c6ccccc6)c6c5c(-c5ccccc5)cc5ccccc56)cc4)n3)cc2)cc1]

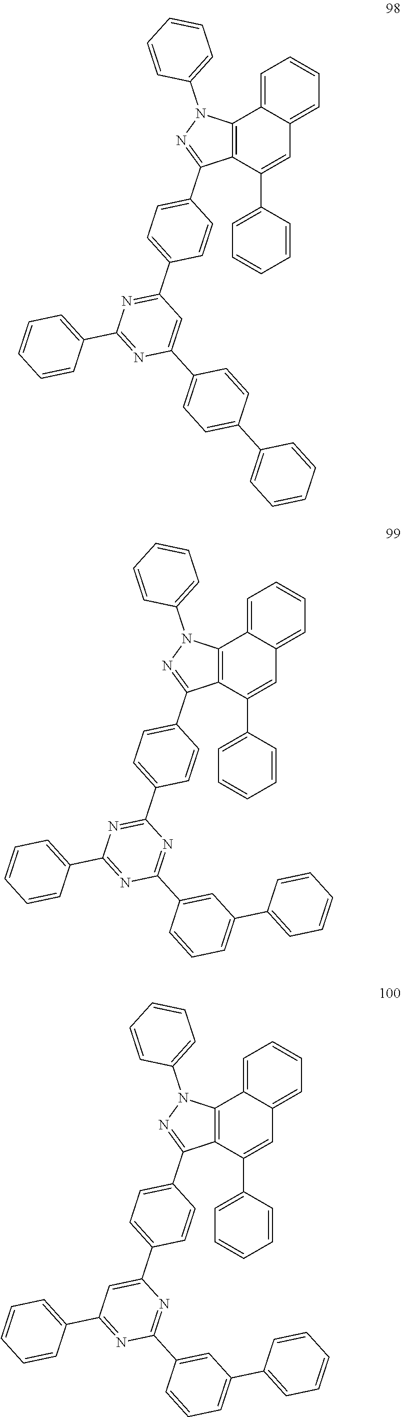 [SMILES: c1ccc(-c2ccc(-c3cc(-c4ccc(-c5nn(-c6ccccc6)c6c5c(-c5ccccc5)cc5ccccc56)cc4)nc(-c4ccccc4)n3)cc2)cc1 ; c1ccc(-c2cccc(-c3nc(-c4ccccc4)nc(-c4ccc(-c5nn(-c6ccccc6)c6c5c(-c5ccccc5)cc5ccccc56)cc4)n3)c2)cc1 ; c1ccc(-c2cccc(-c3nc(-c4ccccc4)cc(-c4ccc(-c5nn(-c6ccccc6)c6c5c(-c5ccccc5)cc5ccccc56)cc4)n3)c2)cc1]

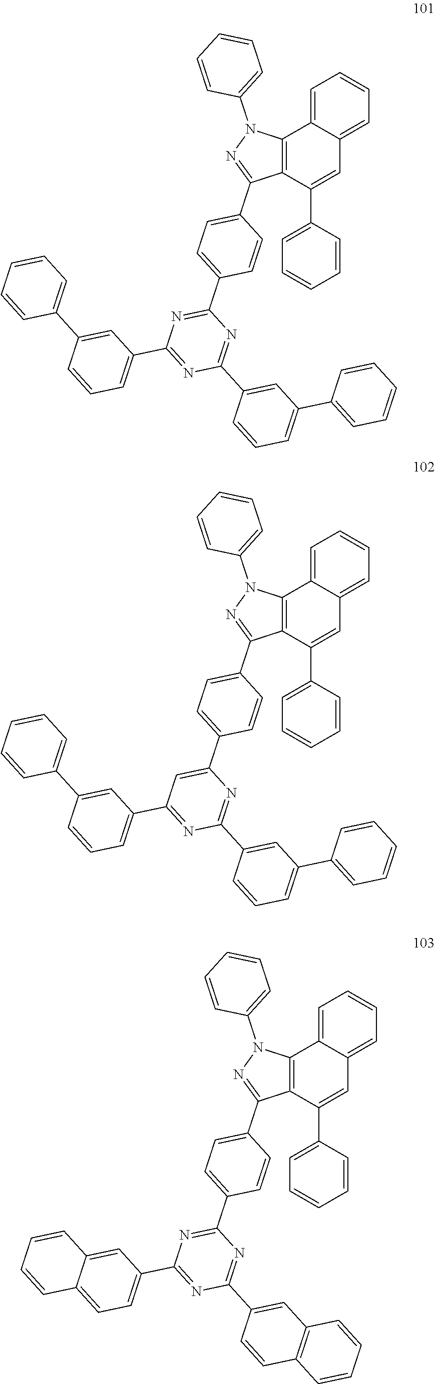 [SMILES: c1ccc(-c2cccc(-c3nc(-c4ccc(-c5nn(-c6ccccc6)c6c5c(-c5ccccc5)cc5ccccc56)cc4)nc(-c4cccc(-c5ccccc5)c4)n3)c2)cc1 ; c1ccc(-c2cccc(-c3cc(-c4ccc(-c5nn(-c6ccccc6)c6c5c(-c5ccccc5)cc5ccccc56)cc4)nc(-c4cccc(-c5ccccc5)c4)n3)c2)cc1 ; c1ccc(-c2cc3ccccc3c3c2c(-c2ccc(-c4nc(-c5ccc6ccccc6c5)nc(-c5ccc6ccccc6c5)n4)cc2)nn3-c2ccccc2)cc1]

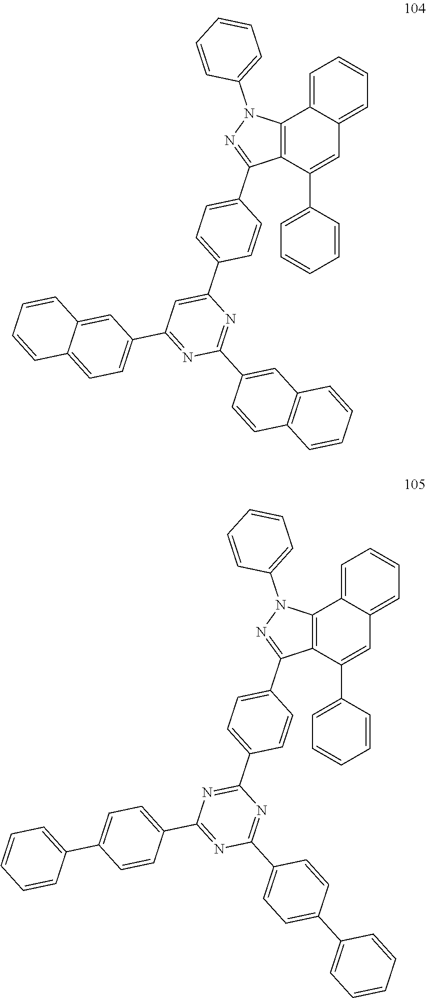 [SMILES: c1ccc(-c2cc3ccccc3c3c2c(-c2ccc(-c4cc(-c5ccc6ccccc6c5)nc(-c5ccc6ccccc6c5)n4)cc2)nn3-c2ccccc2)cc1 ; c1ccc(-c2ccc(-c3nc(-c4ccc(-c5ccccc5)cc4)nc(-c4ccc(-c5nn(-c6ccccc6)c6c5c(-c5ccccc5)cc5ccccc56)cc4)n3)cc2)cc1]

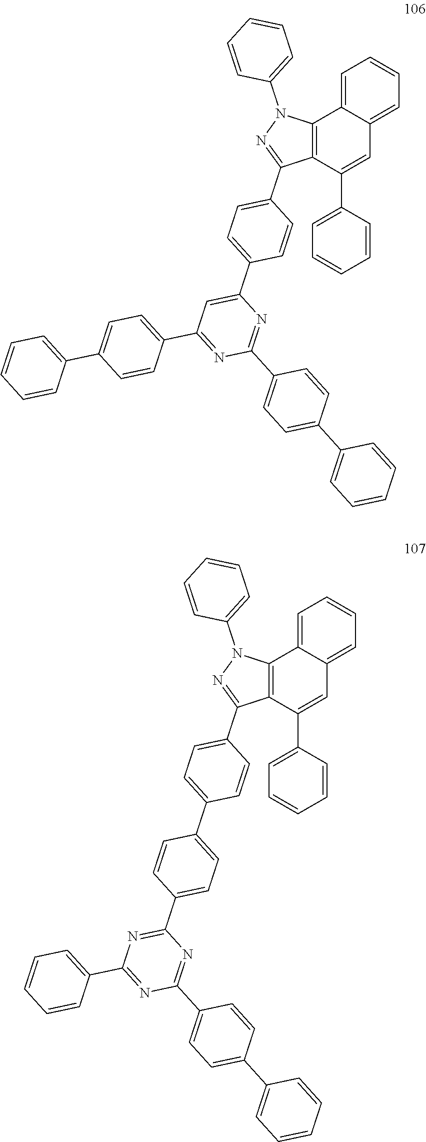 [SMILES: c1ccc(-c2ccc(-c3cc(-c4ccc(-c5nn(-c6ccccc6)c6c5c(-c5ccccc5)cc5ccccc56)cc4)nc(-c4ccc(-c5ccccc5)cc4)n3)cc2)cc1 ; c1ccc(-c2ccc(-c3nc(-c4ccccc4)nc(-c4ccc(-c5ccc(-c6nn(-c7ccccc7)c7c6c(-c6ccccc6)cc6ccccc67)cc5)cc4)n3)cc2)cc1]

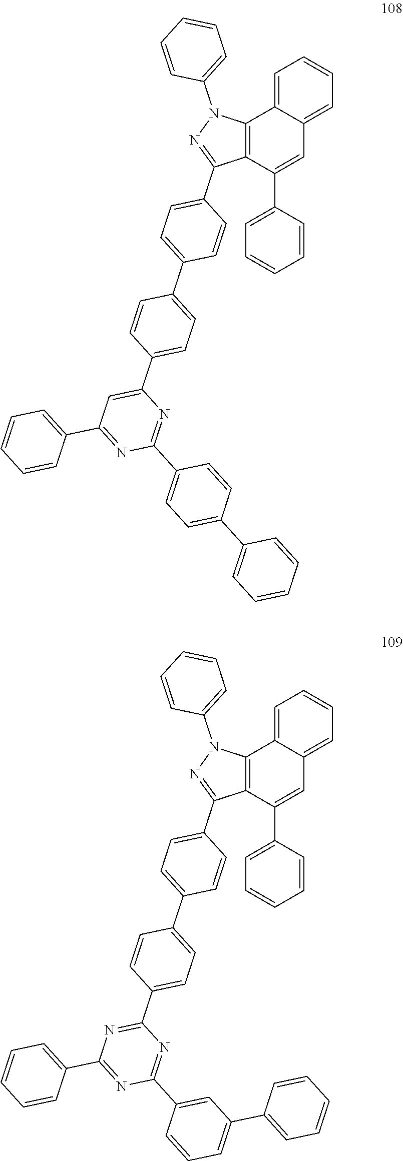 [SMILES: c1ccc(-c2ccc(-c3nc(-c4ccccc4)cc(-c4ccc(-c5ccc(-c6nn(-c7ccccc7)c7c6c(-c6ccccc6)cc6ccccc67)cc5)cc4)n3)cc2)cc1 ; c1ccc(-c2cccc(-c3nc(-c4ccccc4)nc(-c4ccc(-c5ccc(-c6nn(-c7ccccc7)c7c6c(-c6ccccc6)cc6ccccc67)cc5)cc4)n3)c2)cc1]

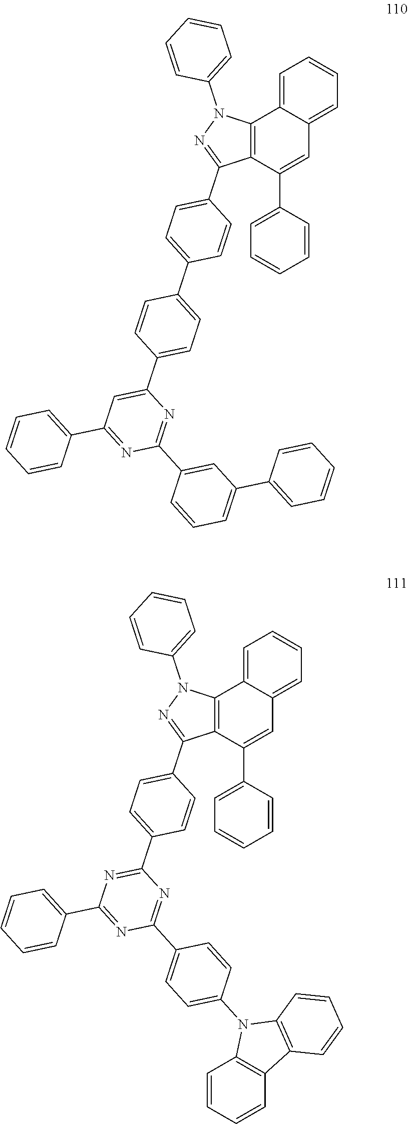 [SMILES: c1ccc(-c2cccc(-c3nc(-c4ccccc4)cc(-c4ccc(-c5ccc(-c6nn(-c7ccccc7)c7c6c(-c6ccccc6)cc6ccccc67)cc5)cc4)n3)c2)cc1 ; c1ccc(-c2nc(-c3ccc(-c4nn(-c5ccccc5)c5c4c(-c4ccccc4)cc4ccccc45)cc3)nc(-c3ccc(-n4c5ccccc5c5ccccc54)cc3)n2)cc1]

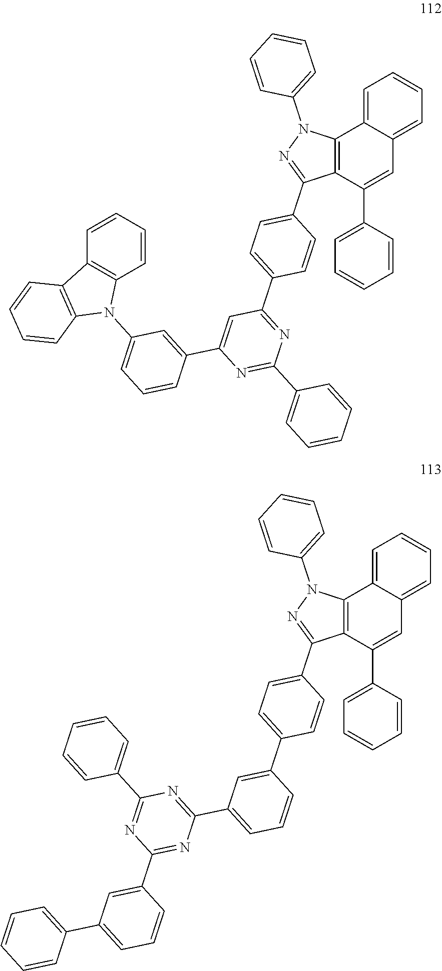 [SMILES: c1ccc(-c2nc(-c3ccc(-c4nn(-c5ccccc5)c5c4c(-c4ccccc4)cc4ccccc45)cc3)cc(-c3cccc(-n4c5ccccc5c5ccccc54)c3)n2)cc1 ; c1ccc(-c2cccc(-c3nc(-c4ccccc4)nc(-c4cccc(-c5ccc(-c6nn(-c7ccccc7)c7c6c(-c6ccccc6)cc6ccccc67)cc5)c4)n3)c2)cc1]

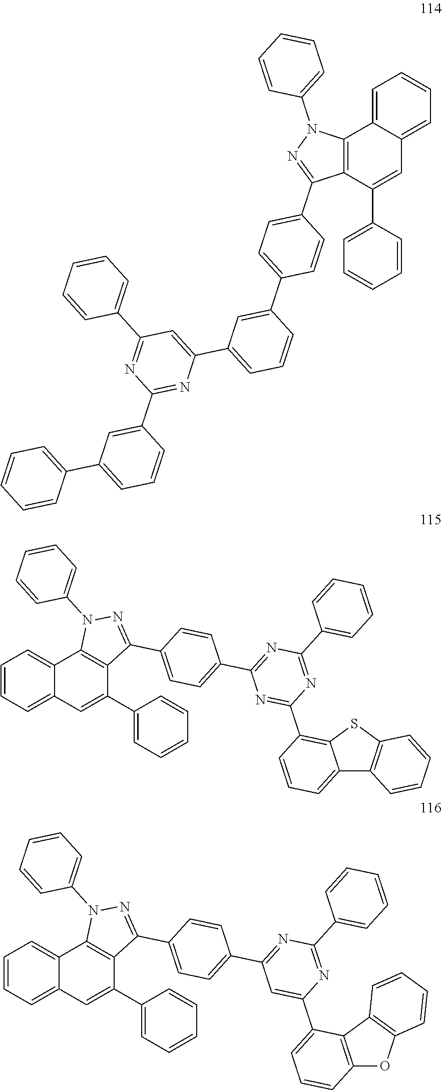

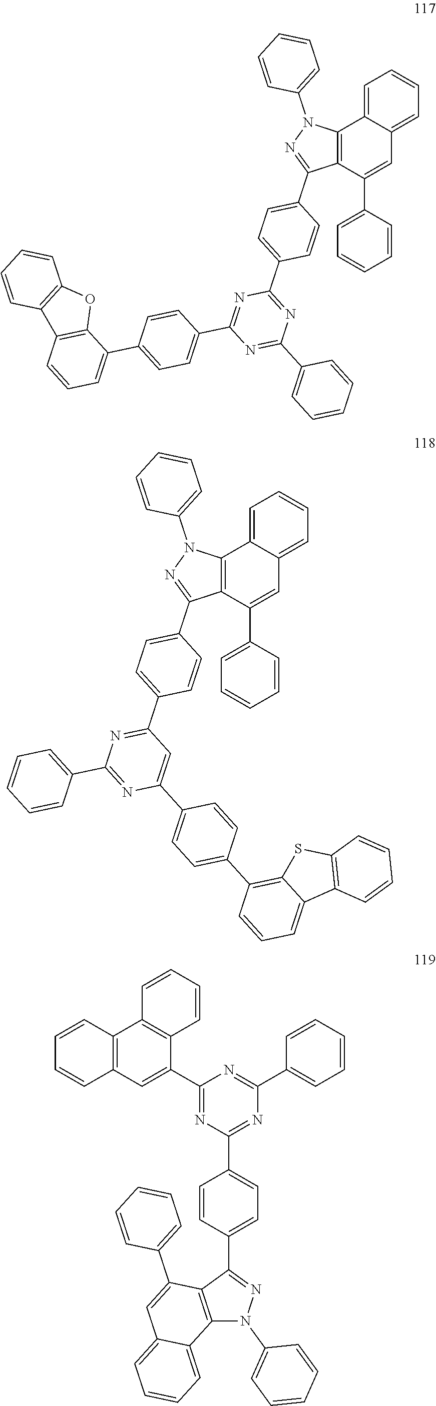 [SMILES: c1ccc(-c2nc(-c3ccc(-c4cccc5c4oc4ccccc45)cc3)nc(-c3ccc(-c4nn(-c5ccccc5)c5c4c(-c4ccccc4)cc4ccccc45)cc3)n2)cc1 ; c1ccc(-c2nc(-c3ccc(-c4cccc5c4sc4ccccc45)cc3)cc(-c3ccc(-c4nn(-c5ccccc5)c5c4c(-c4ccccc4)cc4ccccc45)cc3)n2)cc1 ; c1ccc(-c2nc(-c3ccc(-c4nn(-c5ccccc5)c5c4c(-c4ccccc4)cc4ccccc45)cc3)nc(-c3cc4ccccc4c4ccccc34)n2)cc1]

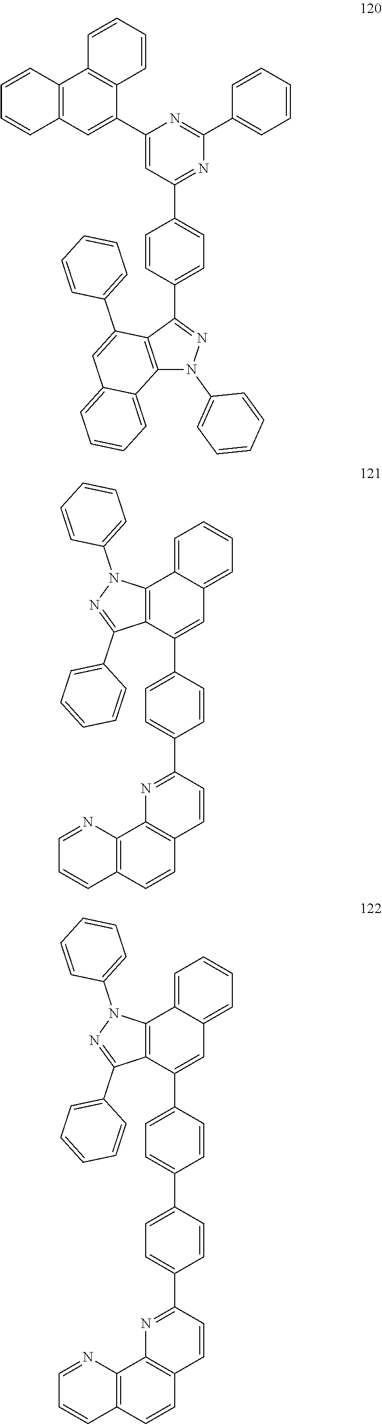

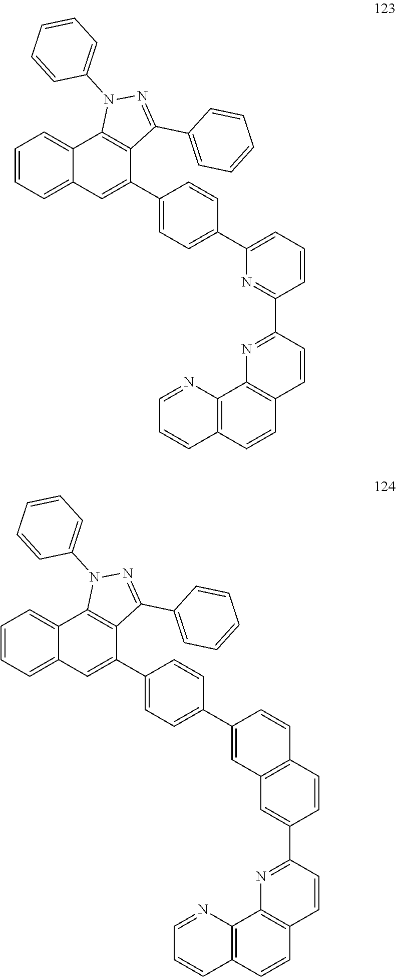 [SMILES: c1ccc(-c2nn(-c3ccccc3)c3c2c(-c2ccc(-c4cccc(-c5ccc6ccc7cccnc7c6n5)n4)cc2)cc2ccccc23)cc1 ; c1ccc(-c2nn(-c3ccccc3)c3c2c(-c2ccc(-c4ccc5ccc(-c6ccc7ccc8cccnc8c7n6)cc5c4)cc2)cc2ccccc23)cc1]

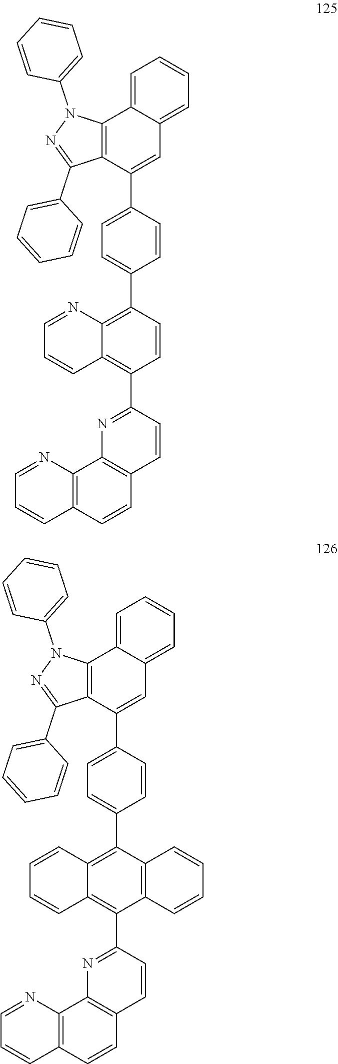

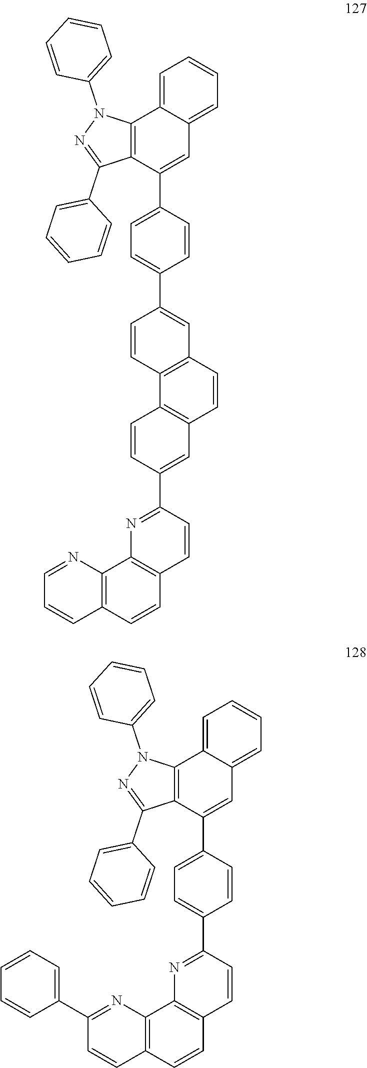

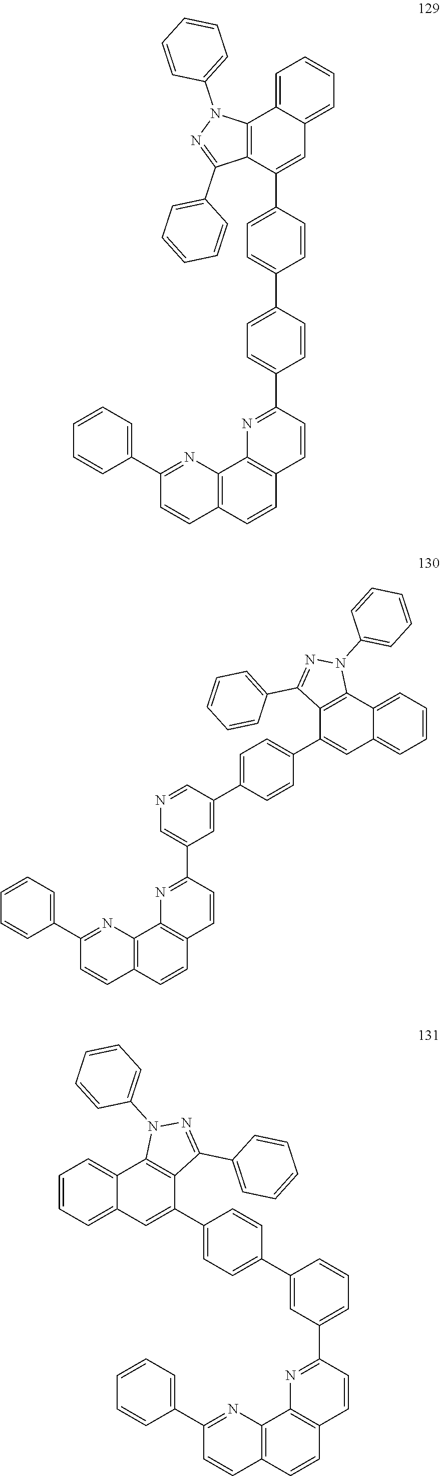 [SMILES: c1ccc(-c2ccc3ccc4ccc(-c5ccc(-c6ccc(-c7cc8ccccc8c8c7c(-c7ccccc7)nn8-c7ccccc7)cc6)cc5)nc4c3n2)cc1 ; c1ccc(-c2ccc3ccc4ccc(-c5cncc(-c6ccc(-c7cc8ccccc8c8c7c(-c7ccccc7)nn8-c7ccccc7)cc6)c5)nc4c3n2)cc1 ; c1ccc(-c2ccc3ccc4ccc(-c5cccc(-c6ccc(-c7cc8ccccc8c8c7c(-c7ccccc7)nn8-c7ccccc7)cc6)c5)nc4c3n2)cc1]

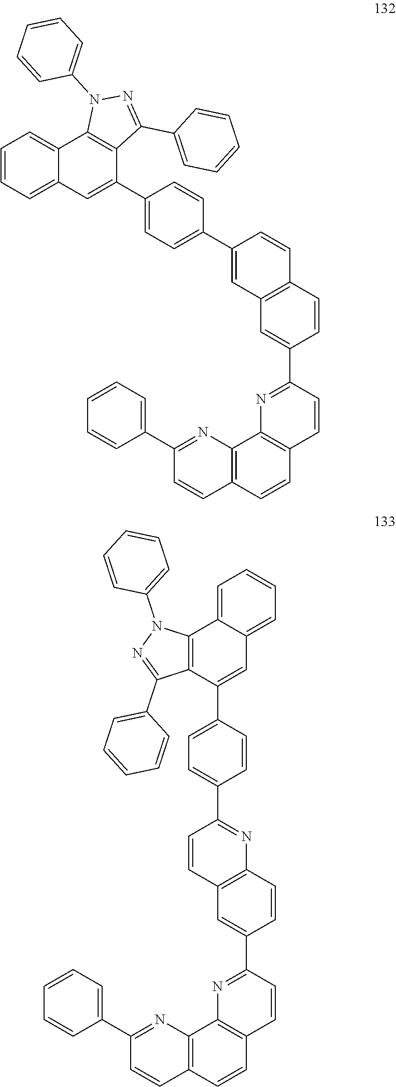 [SMILES: c1ccc(-c2ccc3ccc4ccc(-c5ccc6ccc(-c7ccc(-c8cc9ccccc9c9c8c(-c8ccccc8)nn9-c8ccccc8)cc7)cc6c5)nc4c3n2)cc1 ; c1ccc(-c2ccc3ccc4ccc(-c5ccc6nc(-c7ccc(-c8cc9ccccc9c9c8c(-c8ccccc8)nn9-c8ccccc8)cc7)ccc6c5)nc4c3n2)cc1]

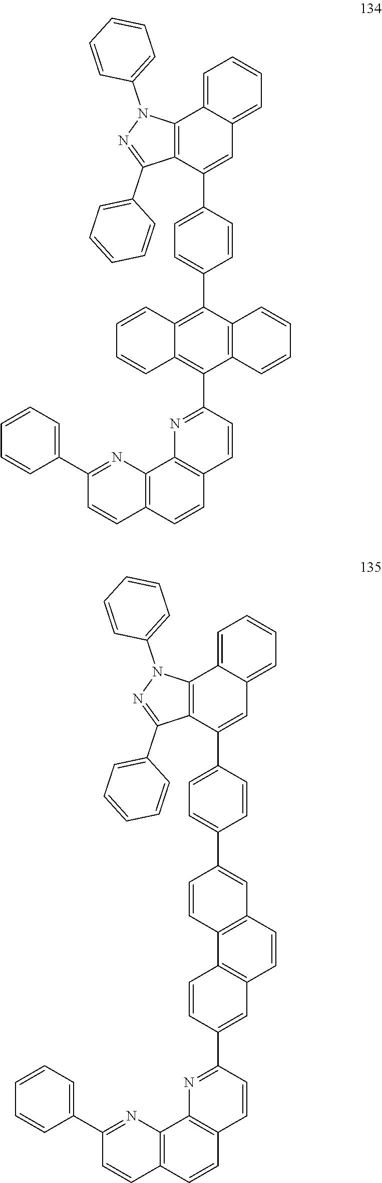 [SMILES: c1ccc(-c2ccc3ccc4ccc(-c5c6ccccc6c(-c6ccc(-c7cc8ccccc8c8c7c(-c7ccccc7)nn8-c7ccccc7)cc6)c6ccccc56)nc4c3n2)cc1 ; c1ccc(-c2ccc3ccc4ccc(-c5ccc6c(ccc7cc(-c8ccc(-c9cc%10ccccc%10c%10c9c(-c9ccccc9)nn%10-c9ccccc9)cc8)ccc76)c5)nc4c3n2)cc1]

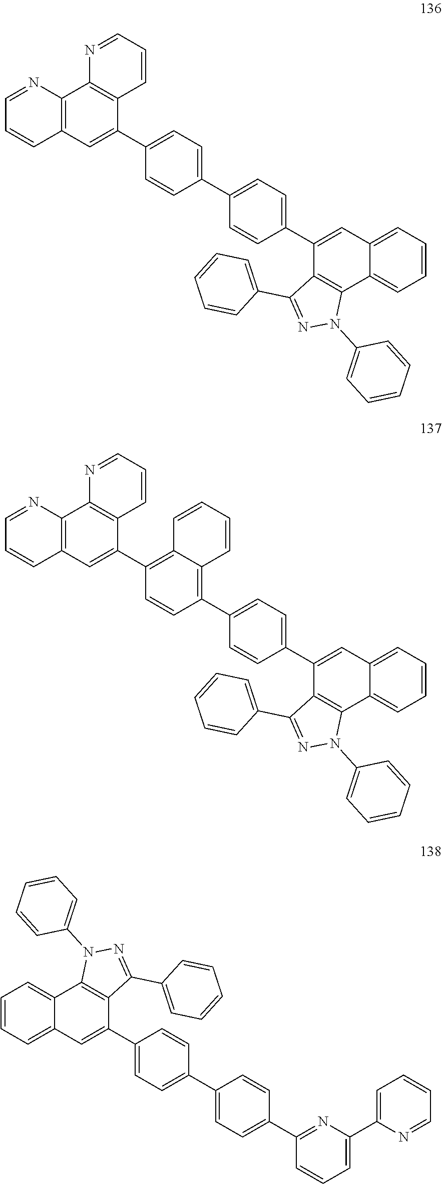 [SMILES: c1ccc(-c2nn(-c3ccccc3)c3c2c(-c2ccc(-c4ccc(-c5cc6cccnc6c6ncccc56)cc4)cc2)cc2ccccc23)cc1 ; c1ccc(-c2nn(-c3ccccc3)c3c2c(-c2ccc(-c4ccc(-c5cc6cccnc6c6ncccc56)c5ccccc45)cc2)cc2ccccc23)cc1 ; c1ccc(-c2nn(-c3ccccc3)c3c2c(-c2ccc(-c4ccc(-c5cccc(-c6ccccn6)n5)cc4)cc2)cc2ccccc23)cc1]

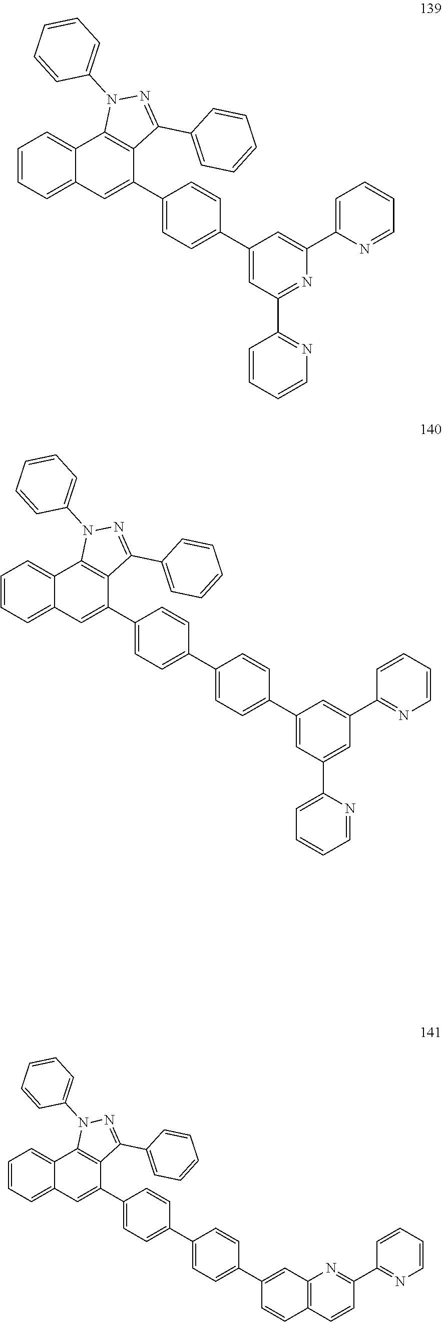 [SMILES: c1ccc(-c2nn(-c3ccccc3)c3c2c(-c2ccc(-c4cc(-c5ccccn5)nc(-c5ccccn5)c4)cc2)cc2ccccc23)cc1 ; c1ccc(-c2nn(-c3ccccc3)c3c2c(-c2ccc(-c4ccc(-c5cc(-c6ccccn6)cc(-c6ccccn6)c5)cc4)cc2)cc2ccccc23)cc1 ; c1ccc(-c2nn(-c3ccccc3)c3c2c(-c2ccc(-c4ccc(-c5ccc6ccc(-c7ccccn7)nc6c5)cc4)cc2)cc2ccccc23)cc1]

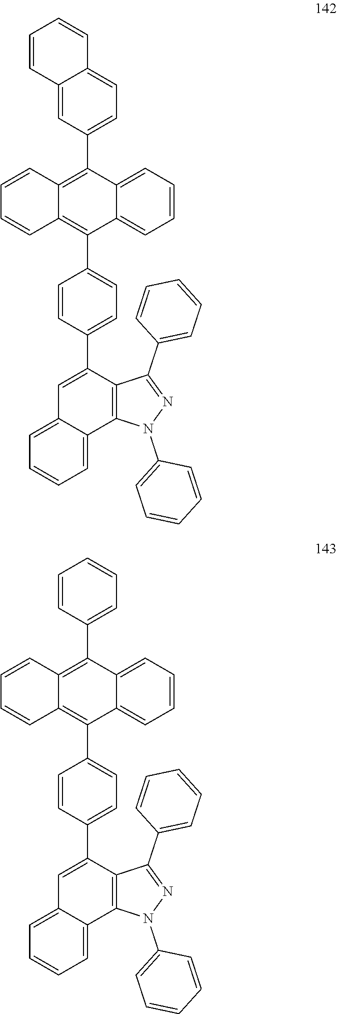 [SMILES: c1ccc(-c2nn(-c3ccccc3)c3c2c(-c2ccc(-c4c5ccccc5c(-c5ccc6ccccc6c5)c5ccccc45)cc2)cc2ccccc23)cc1 ; c1ccc(-c2c3ccccc3c(-c3ccc(-c4cc5ccccc5c5c4c(-c4ccccc4)nn5-c4ccccc4)cc3)c3ccccc23)cc1]

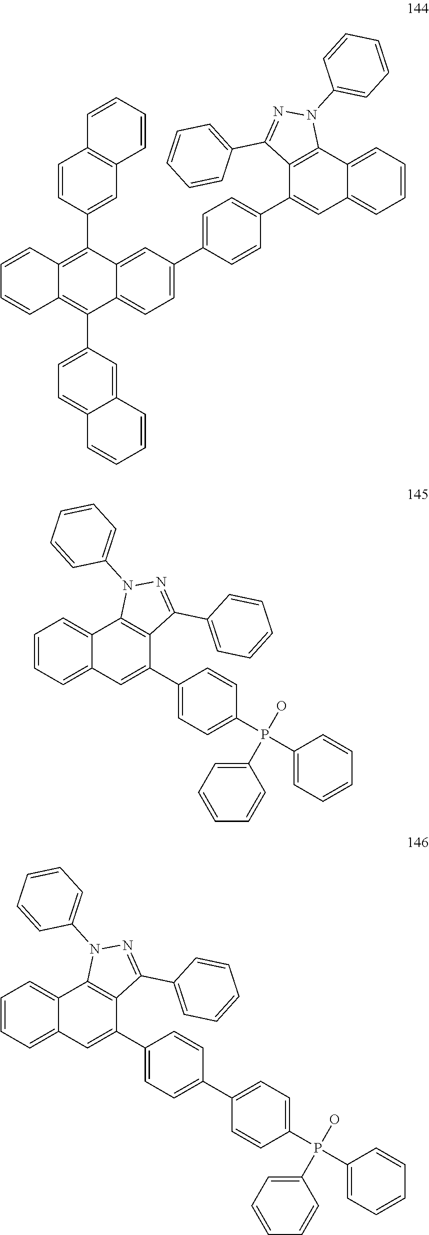

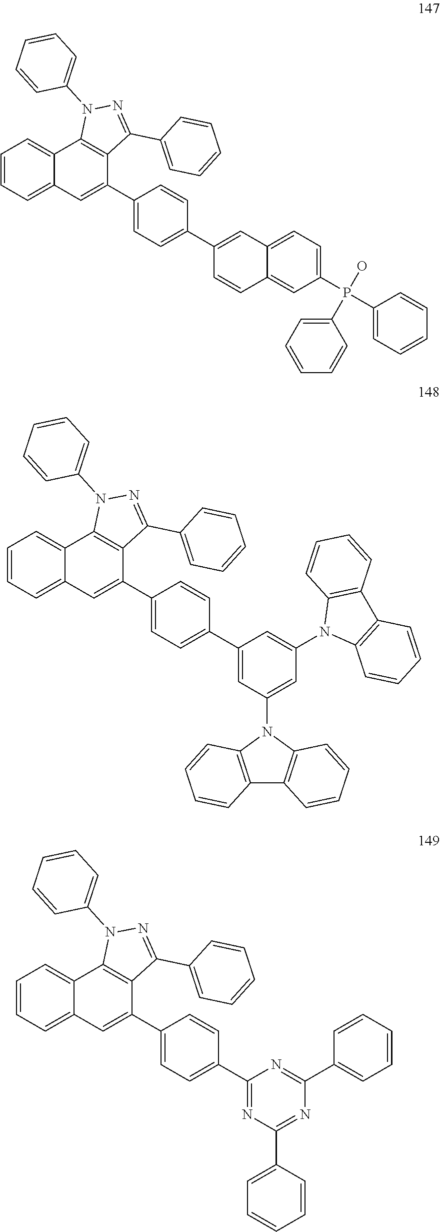

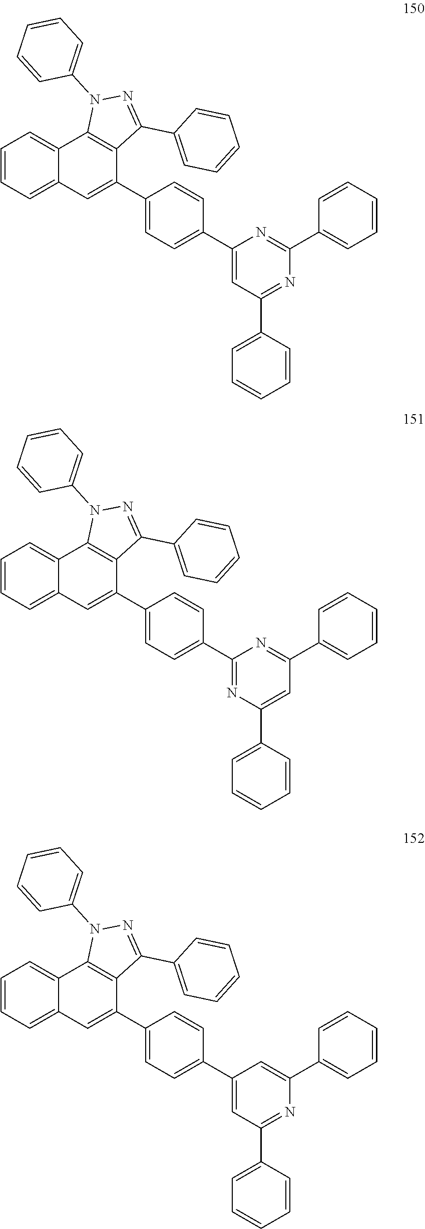 [SMILES: c1ccc(-c2cc(-c3ccc(-c4cc5ccccc5c5c4c(-c4ccccc4)nn5-c4ccccc4)cc3)nc(-c3ccccc3)n2)cc1 ; c1ccc(-c2cc(-c3ccccc3)nc(-c3ccc(-c4cc5ccccc5c5c4c(-c4ccccc4)nn5-c4ccccc4)cc3)n2)cc1 ; c1ccc(-c2cc(-c3ccc(-c4cc5ccccc5c5c4c(-c4ccccc4)nn5-c4ccccc4)cc3)cc(-c3ccccc3)n2)cc1]

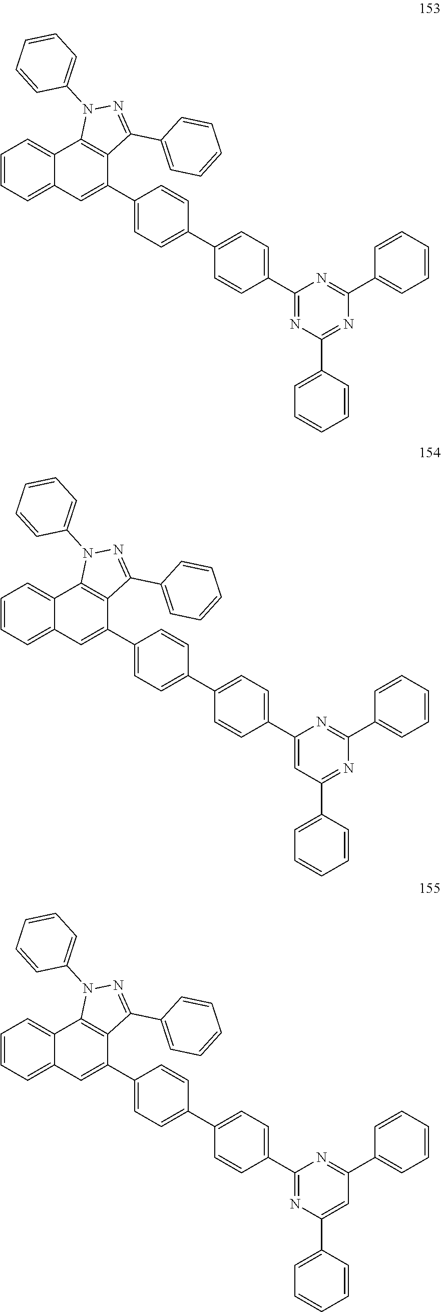 [SMILES: c1ccc(-c2nc(-c3ccccc3)nc(-c3ccc(-c4ccc(-c5cc6ccccc6c6c5c(-c5ccccc5)nn6-c5ccccc5)cc4)cc3)n2)cc1 ; c1ccc(-c2cc(-c3ccc(-c4ccc(-c5cc6ccccc6c6c5c(-c5ccccc5)nn6-c5ccccc5)cc4)cc3)nc(-c3ccccc3)n2)cc1 ; c1ccc(-c2cc(-c3ccccc3)nc(-c3ccc(-c4ccc(-c5cc6ccccc6c6c5c(-c5ccccc5)nn6-c5ccccc5)cc4)cc3)n2)cc1]

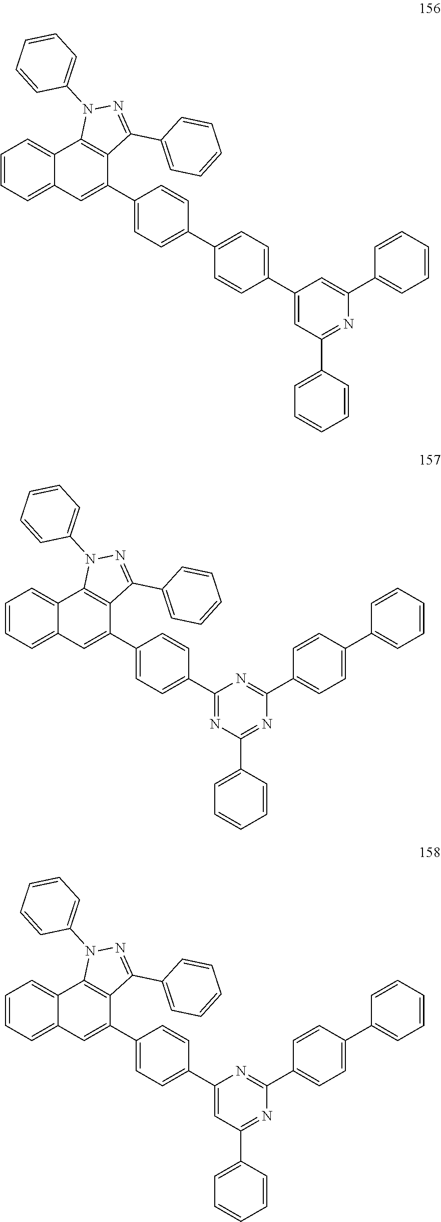 [SMILES: c1ccc(-c2cc(-c3ccc(-c4ccc(-c5cc6ccccc6c6c5c(-c5ccccc5)nn6-c5ccccc5)cc4)cc3)cc(-c3ccccc3)n2)cc1 ; c1ccc(-c2ccc(-c3nc(-c4ccccc4)nc(-c4ccc(-c5cc6ccccc6c6c5c(-c5ccccc5)nn6-c5ccccc5)cc4)n3)cc2)cc1 ; c1ccc(-c2ccc(-c3nc(-c4ccccc4)cc(-c4ccc(-c5cc6ccccc6c6c5c(-c5ccccc5)nn6-c5ccccc5)cc4)n3)cc2)cc1]

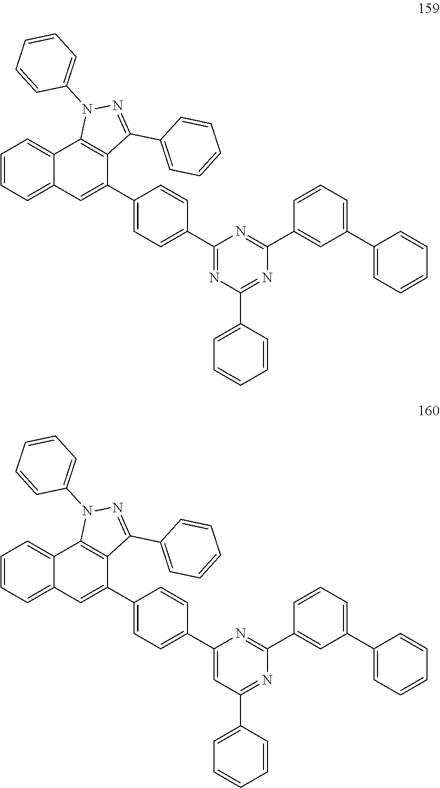 [SMILES: c1ccc(-c2cccc(-c3nc(-c4ccccc4)nc(-c4ccc(-c5cc6ccccc6c6c5c(-c5ccccc5)nn6-c5ccccc5)cc4)n3)c2)cc1 ; c1ccc(-c2cccc(-c3nc(-c4ccccc4)cc(-c4ccc(-c5cc6ccccc6c6c5c(-c5ccccc5)nn6-c5ccccc5)cc4)n3)c2)cc1]

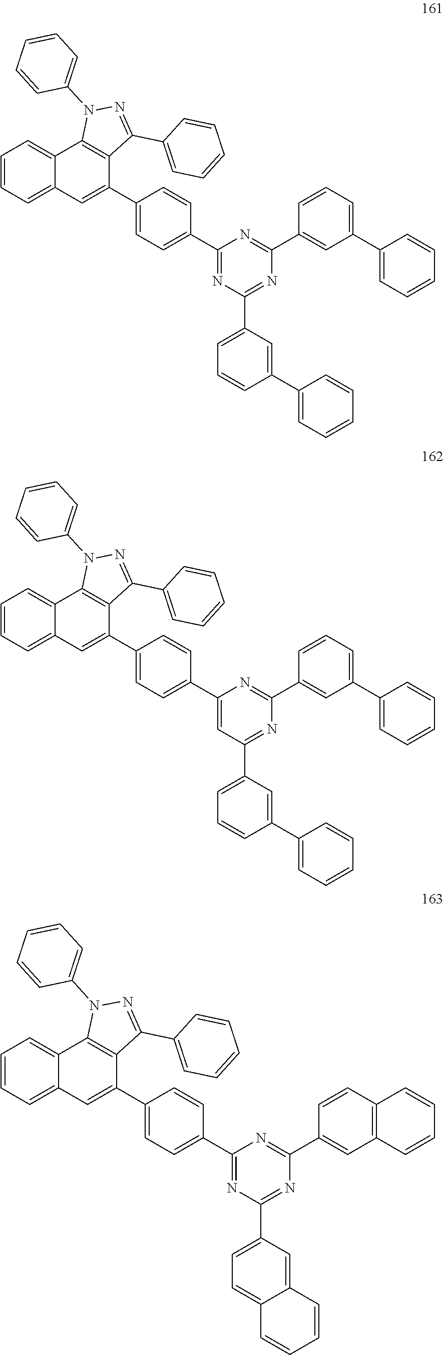 [SMILES: c1ccc(-c2cccc(-c3nc(-c4ccc(-c5cc6ccccc6c6c5c(-c5ccccc5)nn6-c5ccccc5)cc4)nc(-c4cccc(-c5ccccc5)c4)n3)c2)cc1 ; c1ccc(-c2cccc(-c3cc(-c4ccc(-c5cc6ccccc6c6c5c(-c5ccccc5)nn6-c5ccccc5)cc4)nc(-c4cccc(-c5ccccc5)c4)n3)c2)cc1 ; c1ccc(-c2nn(-c3ccccc3)c3c2c(-c2ccc(-c4nc(-c5ccc6ccccc6c5)nc(-c5ccc6ccccc6c5)n4)cc2)cc2ccccc23)cc1]

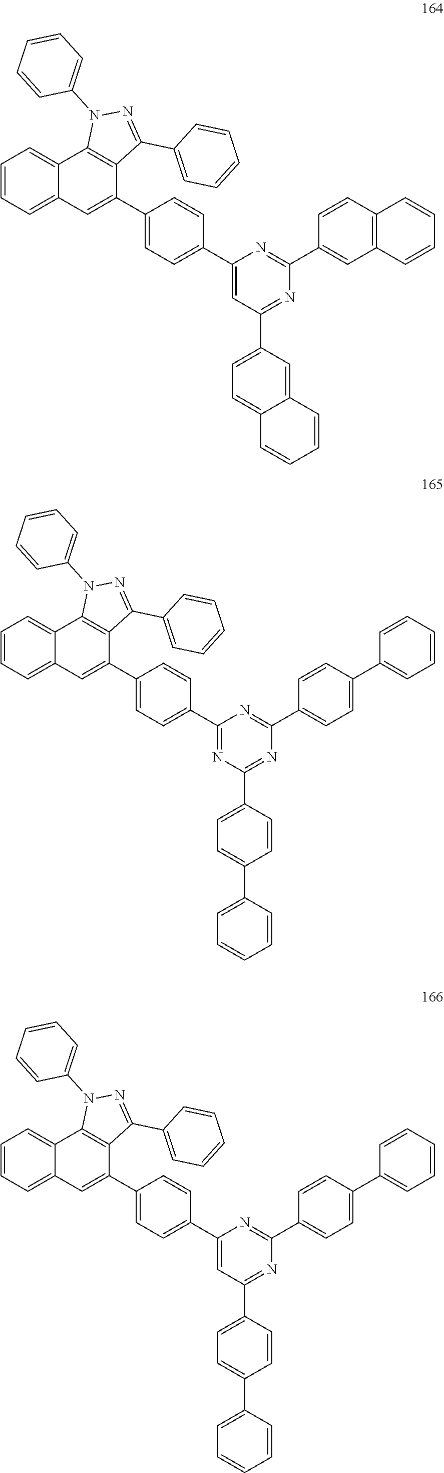 [SMILES: c1ccc(-c2nn(-c3ccccc3)c3c2c(-c2ccc(-c4cc(-c5ccc6ccccc6c5)nc(-c5ccc6ccccc6c5)n4)cc2)cc2ccccc23)cc1 ; c1ccc(-c2ccc(-c3nc(-c4ccc(-c5ccccc5)cc4)nc(-c4ccc(-c5cc6ccccc6c6c5c(-c5ccccc5)nn6-c5ccccc5)cc4)n3)cc2)cc1 ; c1ccc(-c2ccc(-c3cc(-c4ccc(-c5cc6ccccc6c6c5c(-c5ccccc5)nn6-c5ccccc5)cc4)nc(-c4ccc(-c5ccccc5)cc4)n3)cc2)cc1]

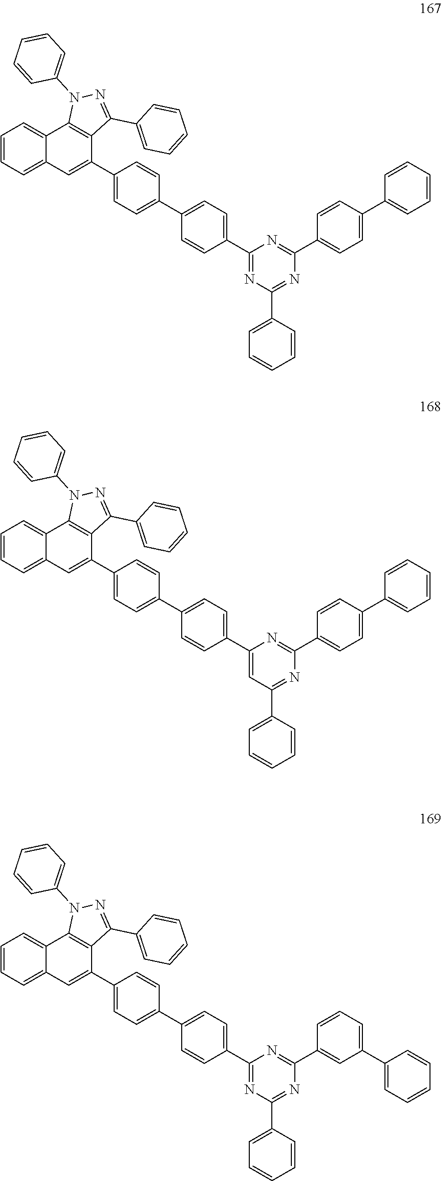 [SMILES: c1ccc(-c2ccc(-c3nc(-c4ccccc4)nc(-c4ccc(-c5ccc(-c6cc7ccccc7c7c6c(-c6ccccc6)nn7-c6ccccc6)cc5)cc4)n3)cc2)cc1 ; c1ccc(-c2ccc(-c3nc(-c4ccccc4)cc(-c4ccc(-c5ccc(-c6cc7ccccc7c7c6c(-c6ccccc6)nn7-c6ccccc6)cc5)cc4)n3)cc2)cc1 ; c1ccc(-c2cccc(-c3nc(-c4ccccc4)nc(-c4ccc(-c5ccc(-c6cc7ccccc7c7c6c(-c6ccccc6)nn7-c6ccccc6)cc5)cc4)n3)c2)cc1]

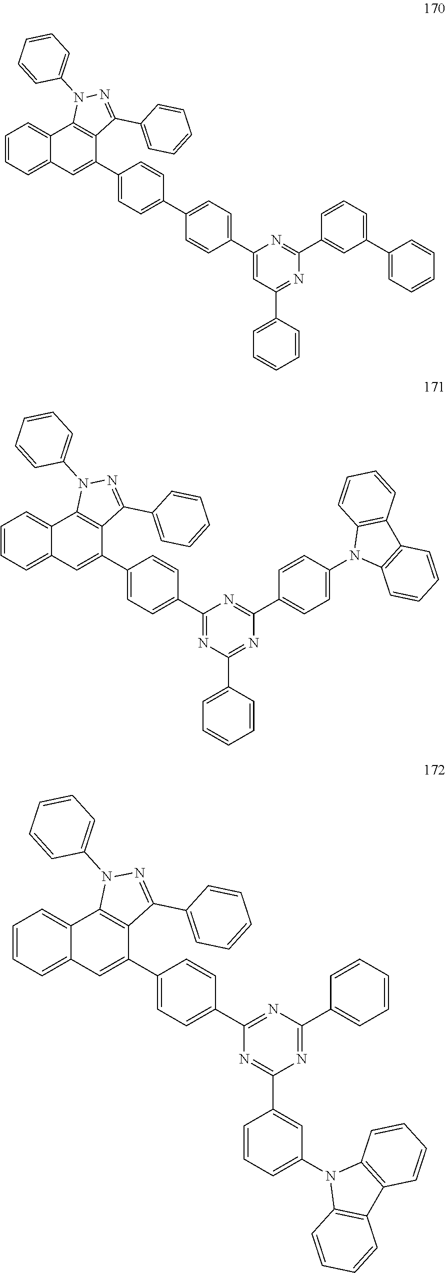 [SMILES: c1ccc(-c2cccc(-c3nc(-c4ccccc4)cc(-c4ccc(-c5ccc(-c6cc7ccccc7c7c6c(-c6ccccc6)nn7-c6ccccc6)cc5)cc4)n3)c2)cc1 ; c1ccc(-c2nc(-c3ccc(-c4cc5ccccc5c5c4c(-c4ccccc4)nn5-c4ccccc4)cc3)nc(-c3ccc(-n4c5ccccc5c5ccccc54)cc3)n2)cc1 ; c1ccc(-c2nc(-c3ccc(-c4cc5ccccc5c5c4c(-c4ccccc4)nn5-c4ccccc4)cc3)nc(-c3cccc(-n4c5ccccc5c5ccccc54)c3)n2)cc1]

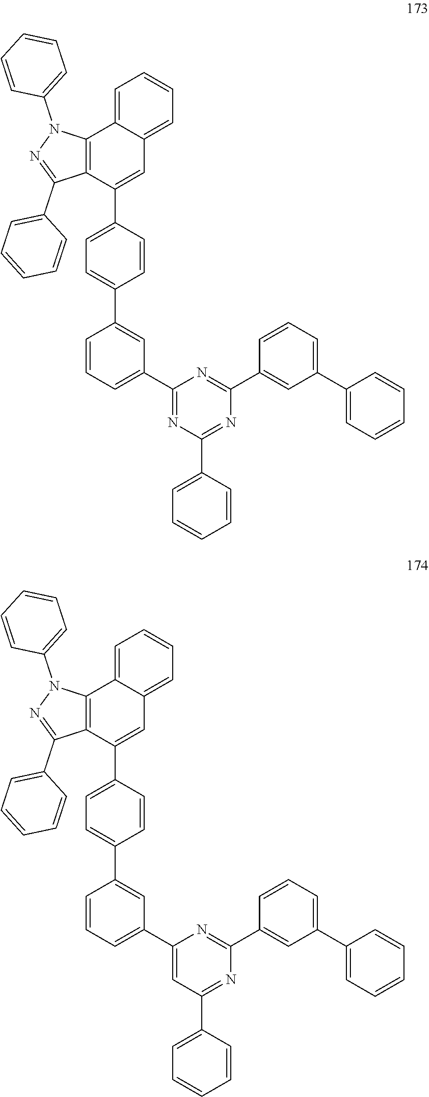 [SMILES: c1ccc(-c2cccc(-c3nc(-c4ccccc4)nc(-c4cccc(-c5ccc(-c6cc7ccccc7c7c6c(-c6ccccc6)nn7-c6ccccc6)cc5)c4)n3)c2)cc1 ; c1ccc(-c2cccc(-c3nc(-c4ccccc4)cc(-c4cccc(-c5ccc(-c6cc7ccccc7c7c6c(-c6ccccc6)nn7-c6ccccc6)cc5)c4)n3)c2)cc1]

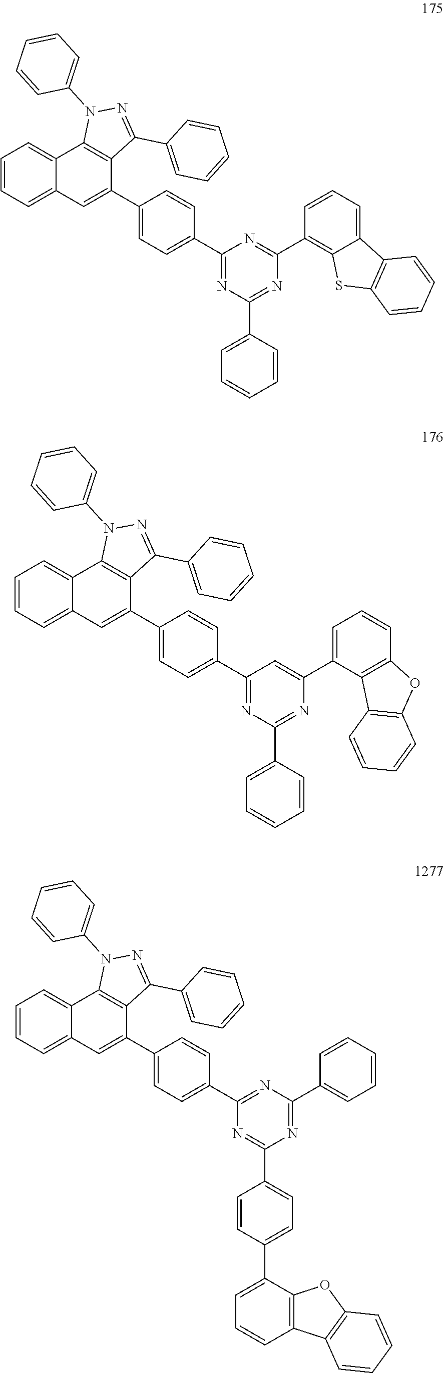 [SMILES: c1ccc(-c2nc(-c3ccc(-c4cc5ccccc5c5c4c(-c4ccccc4)nn5-c4ccccc4)cc3)nc(-c3cccc4c3sc3ccccc34)n2)cc1 ; c1ccc(-c2nc(-c3ccc(-c4cc5ccccc5c5c4c(-c4ccccc4)nn5-c4ccccc4)cc3)cc(-c3cccc4oc5ccccc5c34)n2)cc1 ; c1ccc(-c2nc(-c3ccc(-c4cccc5c4oc4ccccc45)cc3)nc(-c3ccc(-c4cc5ccccc5c5c4c(-c4ccccc4)nn5-c4ccccc4)cc3)n2)cc1]

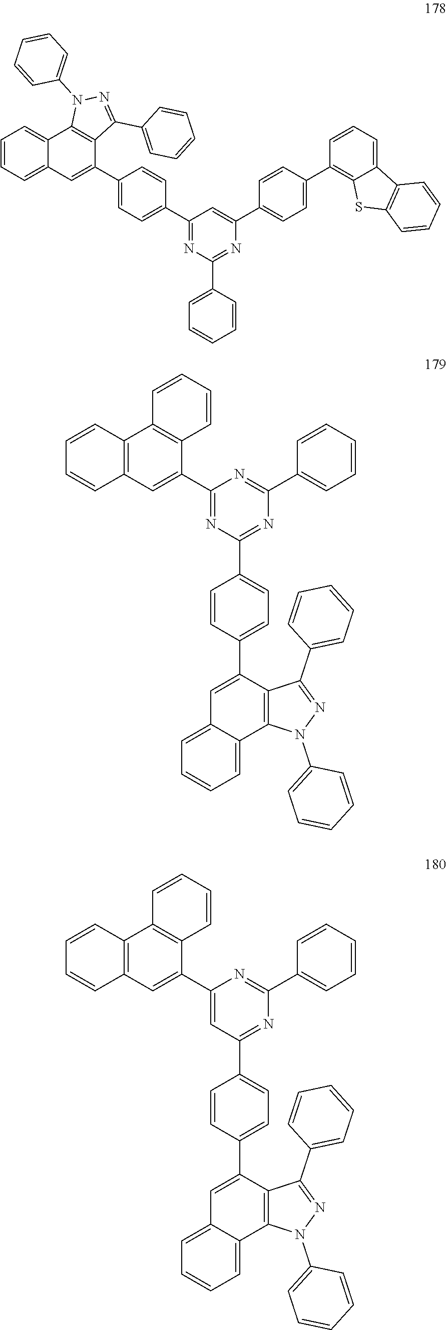

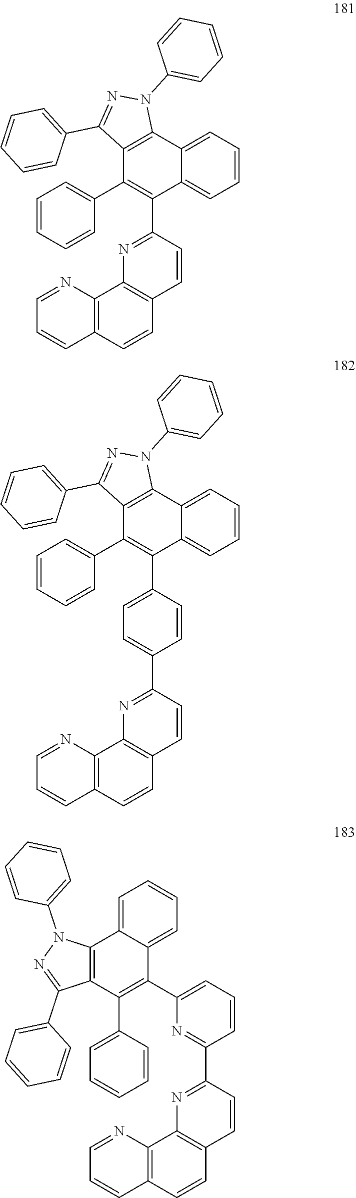

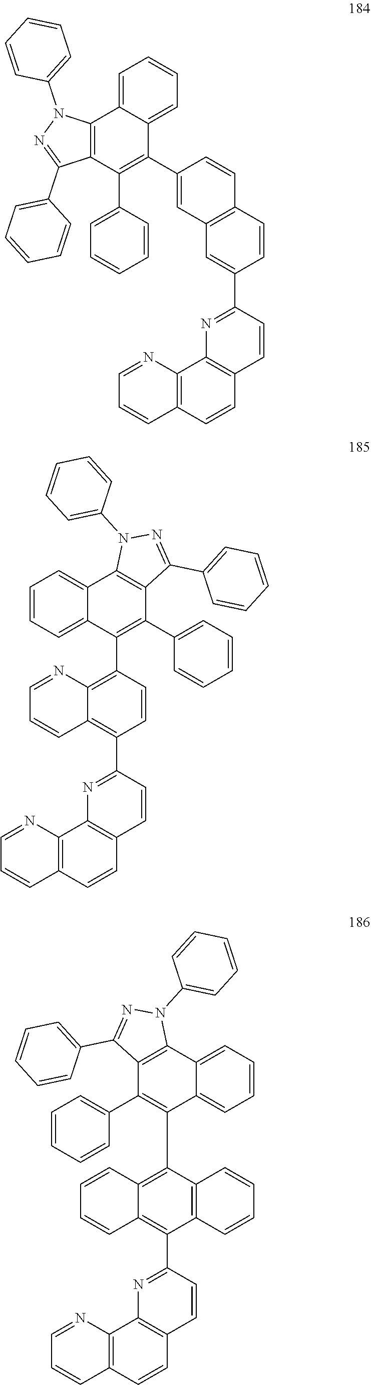 [SMILES: c1ccc(-c2nn(-c3ccccc3)c3c2c(-c2ccccc2)c(-c2ccc4ccc(-c5ccc6ccc7cccnc7c6n5)cc4c2)c2ccccc23)cc1 ; c1ccc(-c2nn(-c3ccccc3)c3c2c(-c2ccccc2)c(-c2ccc(-c4ccc5ccc6cccnc6c5n4)c4cccnc24)c2ccccc23)cc1 ; c1ccc(-c2nn(-c3ccccc3)c3c2c(-c2ccccc2)c(-c2c4ccccc4c(-c4ccc5ccc6cccnc6c5n4)c4ccccc24)c2ccccc23)cc1]

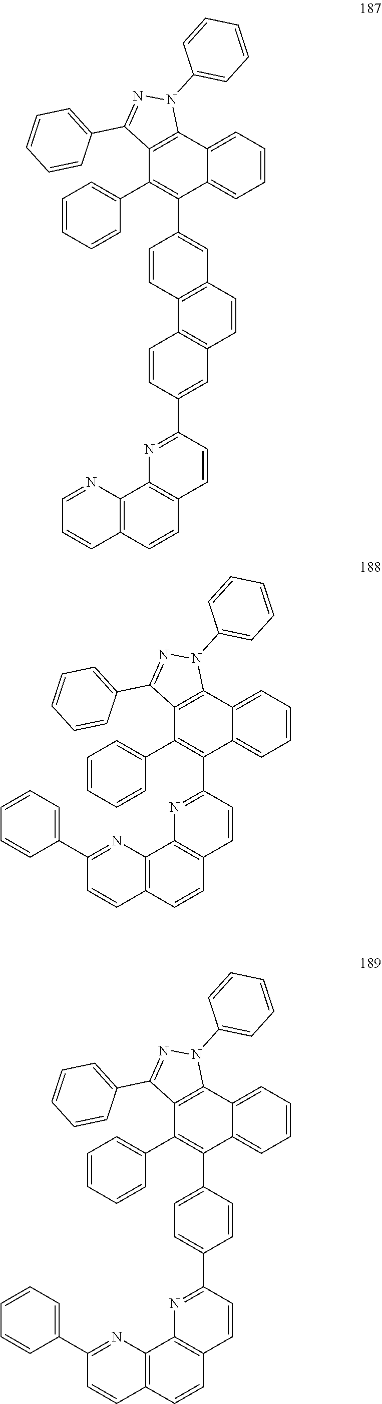 [SMILES: c1ccc(-c2nn(-c3ccccc3)c3c2c(-c2ccccc2)c(-c2ccc4c(ccc5cc(-c6ccc7ccc8cccnc8c7n6)ccc54)c2)c2ccccc23)cc1 ; c1ccc(-c2ccc3ccc4ccc(-c5c(-c6ccccc6)c6c(-c7ccccc7)nn(-c7ccccc7)c6c6ccccc56)nc4c3n2)cc1 ; c1ccc(-c2ccc3ccc4ccc(-c5ccc(-c6c(-c7ccccc7)c7c(-c8ccccc8)nn(-c8ccccc8)c7c7ccccc67)cc5)nc4c3n2)cc1]

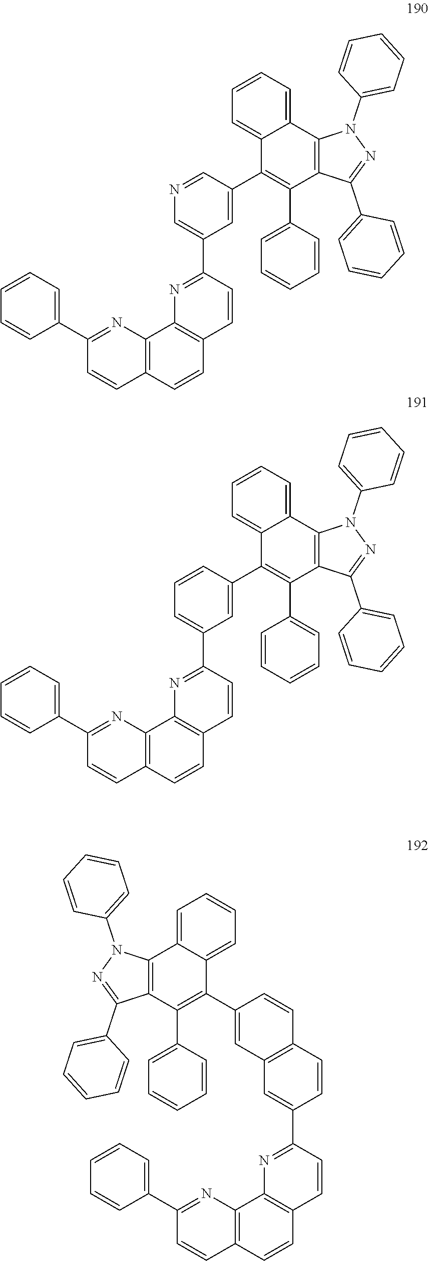 [SMILES: c1ccc(-c2ccc3ccc4ccc(-c5cncc(-c6c(-c7ccccc7)c7c(-c8ccccc8)nn(-c8ccccc8)c7c7ccccc67)c5)nc4c3n2)cc1 ; c1ccc(-c2ccc3ccc4ccc(-c5cccc(-c6c(-c7ccccc7)c7c(-c8ccccc8)nn(-c8ccccc8)c7c7ccccc67)c5)nc4c3n2)cc1 ; c1ccc(-c2ccc3ccc4ccc(-c5ccc6ccc(-c7c(-c8ccccc8)c8c(-c9ccccc9)nn(-c9ccccc9)c8c8ccccc78)cc6c5)nc4c3n2)cc1]

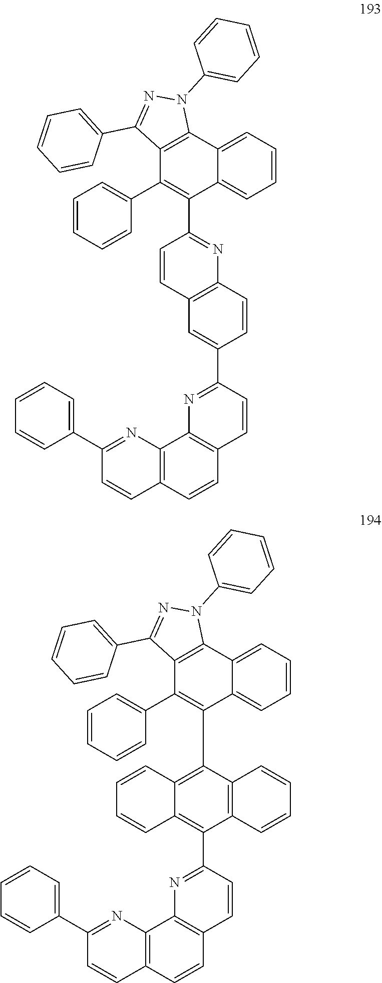 [SMILES: c1ccc(-c2ccc3ccc4ccc(-c5ccc6nc(-c7c(-c8ccccc8)c8c(-c9ccccc9)nn(-c9ccccc9)c8c8ccccc78)ccc6c5)nc4c3n2)cc1 ; c1ccc(-c2ccc3ccc4ccc(-c5c6ccccc6c(-c6c(-c7ccccc7)c7c(-c8ccccc8)nn(-c8ccccc8)c7c7ccccc67)c6ccccc56)nc4c3n2)cc1]

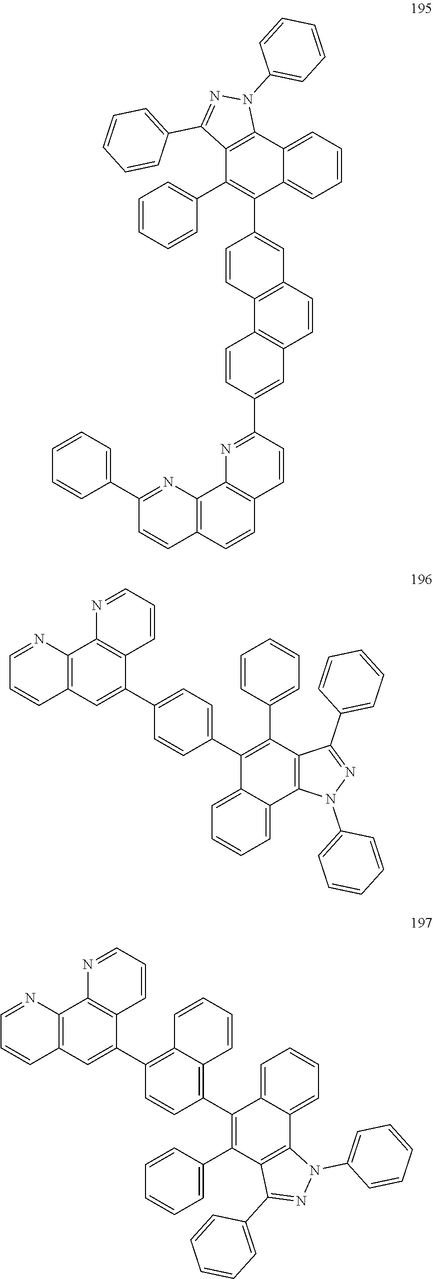 [SMILES: c1ccc(-c2ccc3ccc4ccc(-c5ccc6c(ccc7cc(-c8c(-c9ccccc9)c9c(-c%10ccccc%10)nn(-c%10ccccc%10)c9c9ccccc89)ccc76)c5)nc4c3n2)cc1 ; c1ccc(-c2nn(-c3ccccc3)c3c2c(-c2ccccc2)c(-c2ccc(-c4cc5cccnc5c5ncccc45)cc2)c2ccccc23)cc1 ; c1ccc(-c2nn(-c3ccccc3)c3c2c(-c2ccccc2)c(-c2ccc(-c4cc5cccnc5c5ncccc45)c4ccccc24)c2ccccc23)cc1]

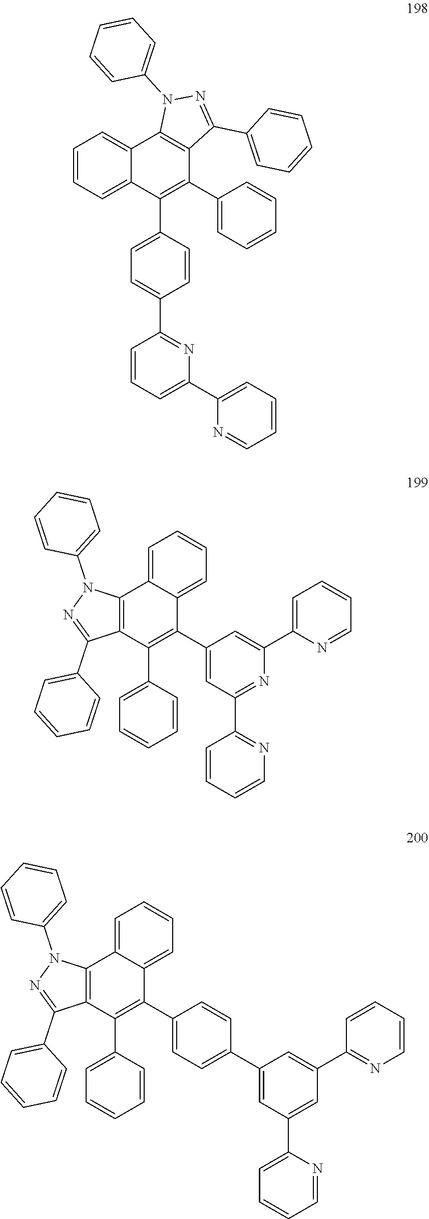 [SMILES: c1ccc(-c2nn(-c3ccccc3)c3c2c(-c2ccccc2)c(-c2ccc(-c4cccc(-c5ccccn5)n4)cc2)c2ccccc23)cc1 ; c1ccc(-c2nn(-c3ccccc3)c3c2c(-c2ccccc2)c(-c2cc(-c4ccccn4)nc(-c4ccccn4)c2)c2ccccc23)cc1 ; c1ccc(-c2nn(-c3ccccc3)c3c2c(-c2ccccc2)c(-c2ccc(-c4cc(-c5ccccn5)cc(-c5ccccn5)c4)cc2)c2ccccc23)cc1]

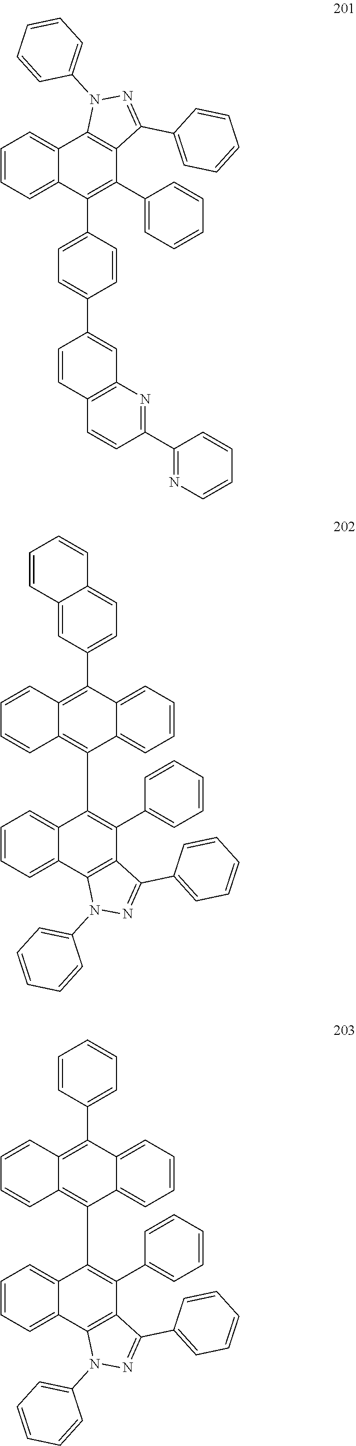 [SMILES: c1ccc(-c2nn(-c3ccccc3)c3c2c(-c2ccccc2)c(-c2ccc(-c4ccc5ccc(-c6ccccn6)nc5c4)cc2)c2ccccc23)cc1 ; c1ccc(-c2nn(-c3ccccc3)c3c2c(-c2ccccc2)c(-c2c4ccccc4c(-c4ccc5ccccc5c4)c4ccccc24)c2ccccc23)cc1 ; c1ccc(-c2c3ccccc3c(-c3c(-c4ccccc4)c4c(-c5ccccc5)nn(-c5ccccc5)c4c4ccccc34)c3ccccc23)cc1]

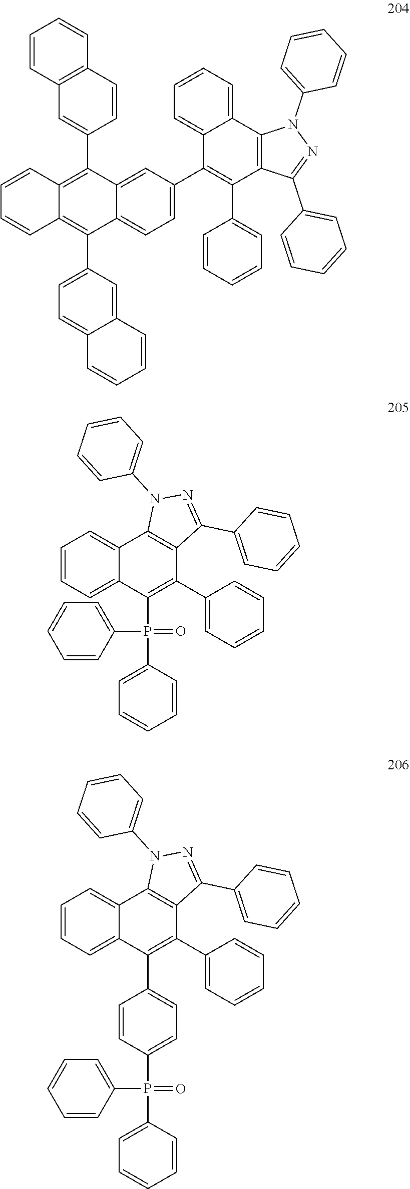

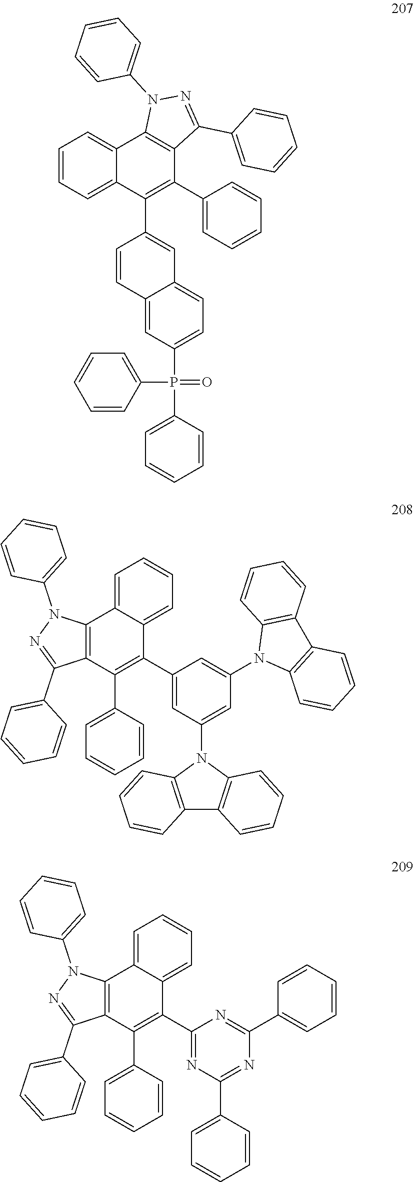 [SMILES: O=P(c1ccccc1)(c1ccccc1)c1ccc2cc(-c3c(-c4ccccc4)c4c(-c5ccccc5)nn(-c5ccccc5)c4c4ccccc34)ccc2c1 ; c1ccc(-c2nn(-c3ccccc3)c3c2c(-c2ccccc2)c(-c2cc(-n4c5ccccc5c5ccccc54)cc(-n4c5ccccc5c5ccccc54)c2)c2ccccc23)cc1 ; c1ccc(-c2nc(-c3ccccc3)nc(-c3c(-c4ccccc4)c4c(-c5ccccc5)nn(-c5ccccc5)c4c4ccccc34)n2)cc1]

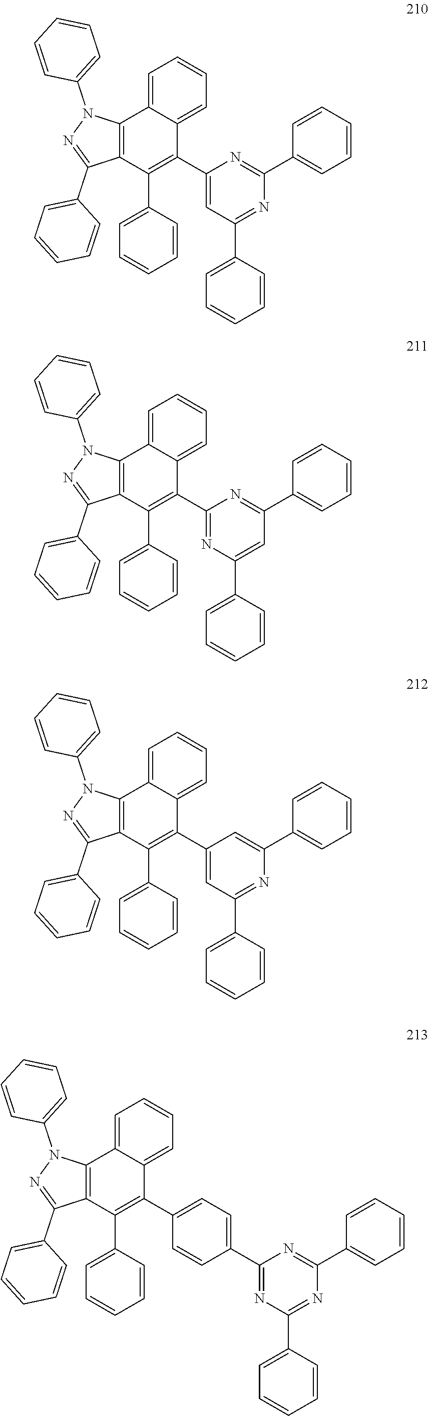 [SMILES: c1ccc(-c2cc(-c3c(-c4ccccc4)c4c(-c5ccccc5)nn(-c5ccccc5)c4c4ccccc34)nc(-c3ccccc3)n2)cc1 ; c1ccc(-c2cc(-c3ccccc3)nc(-c3c(-c4ccccc4)c4c(-c5ccccc5)nn(-c5ccccc5)c4c4ccccc34)n2)cc1 ; c1ccc(-c2cc(-c3c(-c4ccccc4)c4c(-c5ccccc5)nn(-c5ccccc5)c4c4ccccc34)cc(-c3ccccc3)n2)cc1 ; c1ccc(-c2nc(-c3ccccc3)nc(-c3ccc(-c4c(-c5ccccc5)c5c(-c6ccccc6)nn(-c6ccccc6)c5c5ccccc45)cc3)n2)cc1]

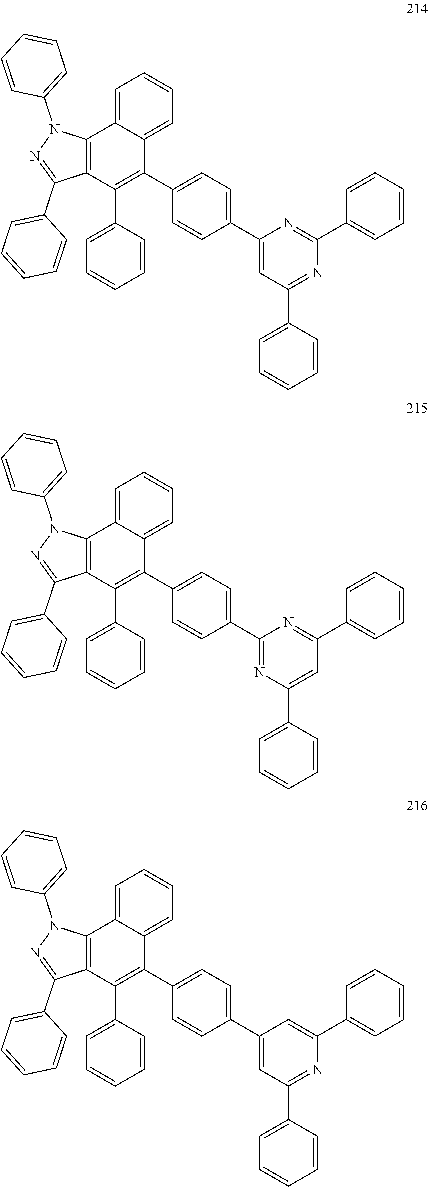 [SMILES: c1ccc(-c2cc(-c3ccc(-c4c(-c5ccccc5)c5c(-c6ccccc6)nn(-c6ccccc6)c5c5ccccc45)cc3)nc(-c3ccccc3)n2)cc1 ; c1ccc(-c2cc(-c3ccccc3)nc(-c3ccc(-c4c(-c5ccccc5)c5c(-c6ccccc6)nn(-c6ccccc6)c5c5ccccc45)cc3)n2)cc1 ; c1ccc(-c2cc(-c3ccc(-c4c(-c5ccccc5)c5c(-c6ccccc6)nn(-c6ccccc6)c5c5ccccc45)cc3)cc(-c3ccccc3)n2)cc1]

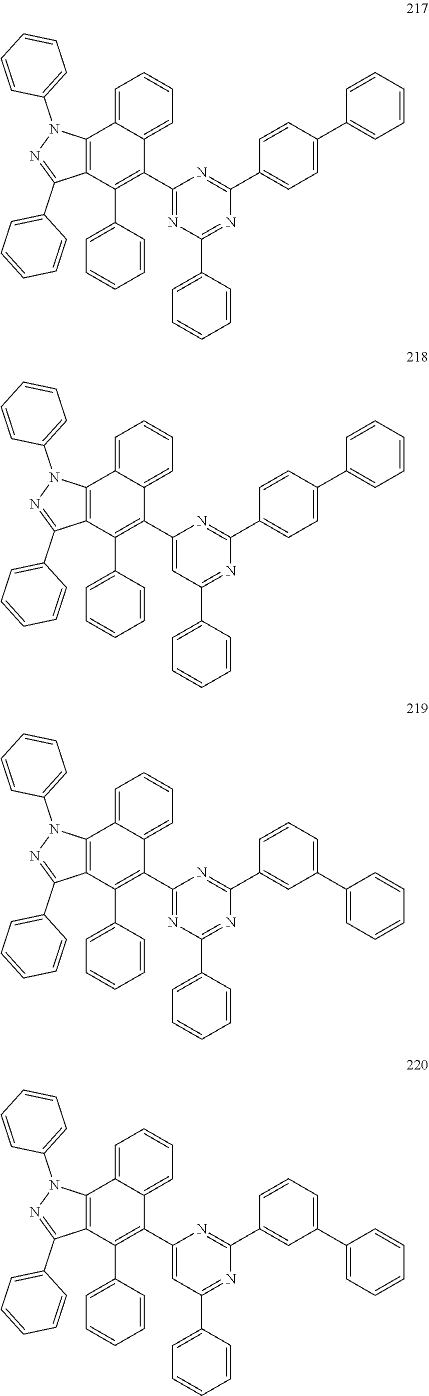 [SMILES: c1ccc(-c2ccc(-c3nc(-c4ccccc4)nc(-c4c(-c5ccccc5)c5c(-c6ccccc6)nn(-c6ccccc6)c5c5ccccc45)n3)cc2)cc1 ; c1ccc(-c2ccc(-c3nc(-c4ccccc4)cc(-c4c(-c5ccccc5)c5c(-c6ccccc6)nn(-c6ccccc6)c5c5ccccc45)n3)cc2)cc1 ; c1ccc(-c2cccc(-c3nc(-c4ccccc4)nc(-c4c(-c5ccccc5)c5c(-c6ccccc6)nn(-c6ccccc6)c5c5ccccc45)n3)c2)cc1 ; c1ccc(-c2cccc(-c3nc(-c4ccccc4)cc(-c4c(-c5ccccc5)c5c(-c6ccccc6)nn(-c6ccccc6)c5c5ccccc45)n3)c2)cc1]

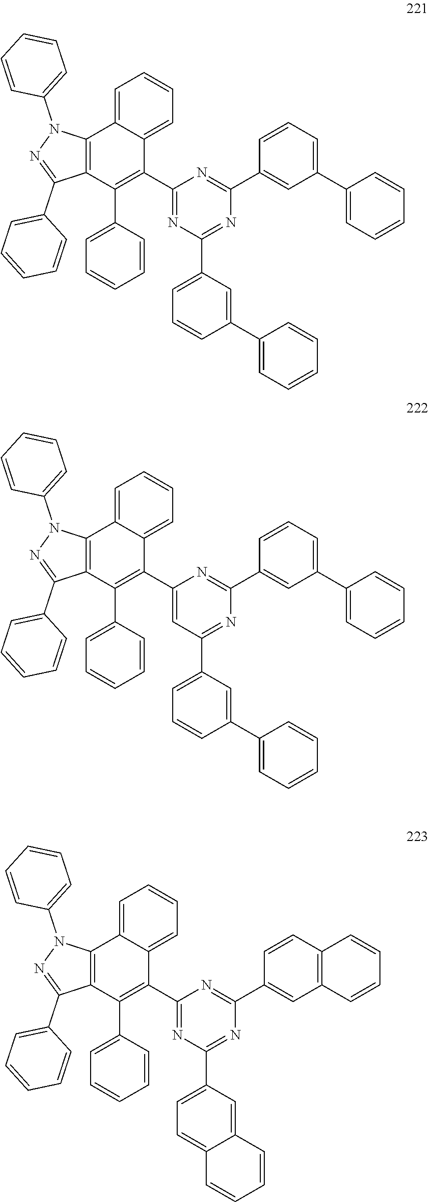 [SMILES: c1ccc(-c2cccc(-c3nc(-c4cccc(-c5ccccc5)c4)nc(-c4c(-c5ccccc5)c5c(-c6ccccc6)nn(-c6ccccc6)c5c5ccccc45)n3)c2)cc1 ; c1ccc(-c2cccc(-c3cc(-c4c(-c5ccccc5)c5c(-c6ccccc6)nn(-c6ccccc6)c5c5ccccc45)nc(-c4cccc(-c5ccccc5)c4)n3)c2)cc1 ; c1ccc(-c2nn(-c3ccccc3)c3c2c(-c2ccccc2)c(-c2nc(-c4ccc5ccccc5c4)nc(-c4ccc5ccccc5c4)n2)c2ccccc23)cc1]

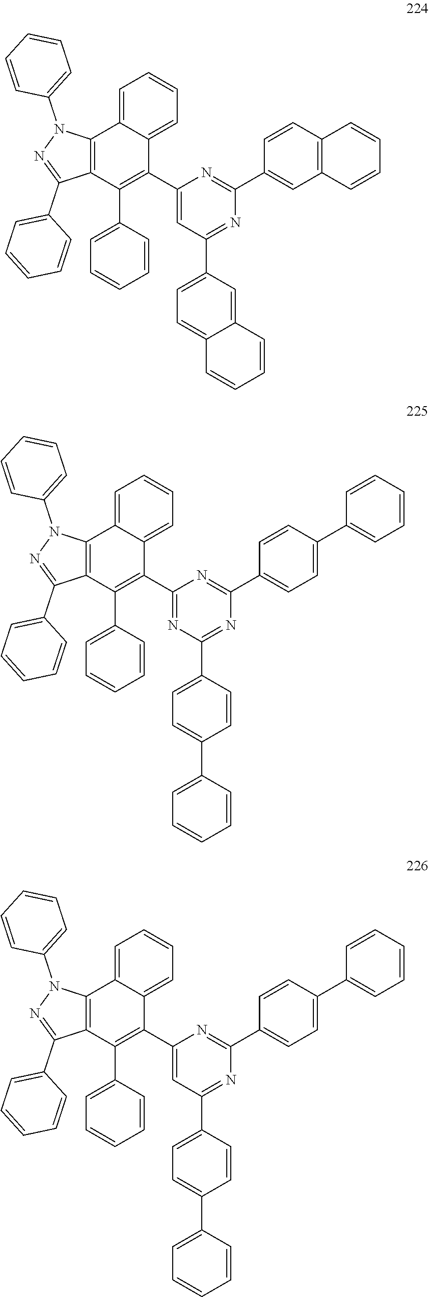 [SMILES: c1ccc(-c2nn(-c3ccccc3)c3c2c(-c2ccccc2)c(-c2cc(-c4ccc5ccccc5c4)nc(-c4ccc5ccccc5c4)n2)c2ccccc23)cc1 ; c1ccc(-c2ccc(-c3nc(-c4ccc(-c5ccccc5)cc4)nc(-c4c(-c5ccccc5)c5c(-c6ccccc6)nn(-c6ccccc6)c5c5ccccc45)n3)cc2)cc1 ; c1ccc(-c2ccc(-c3cc(-c4c(-c5ccccc5)c5c(-c6ccccc6)nn(-c6ccccc6)c5c5ccccc45)nc(-c4ccc(-c5ccccc5)cc4)n3)cc2)cc1]

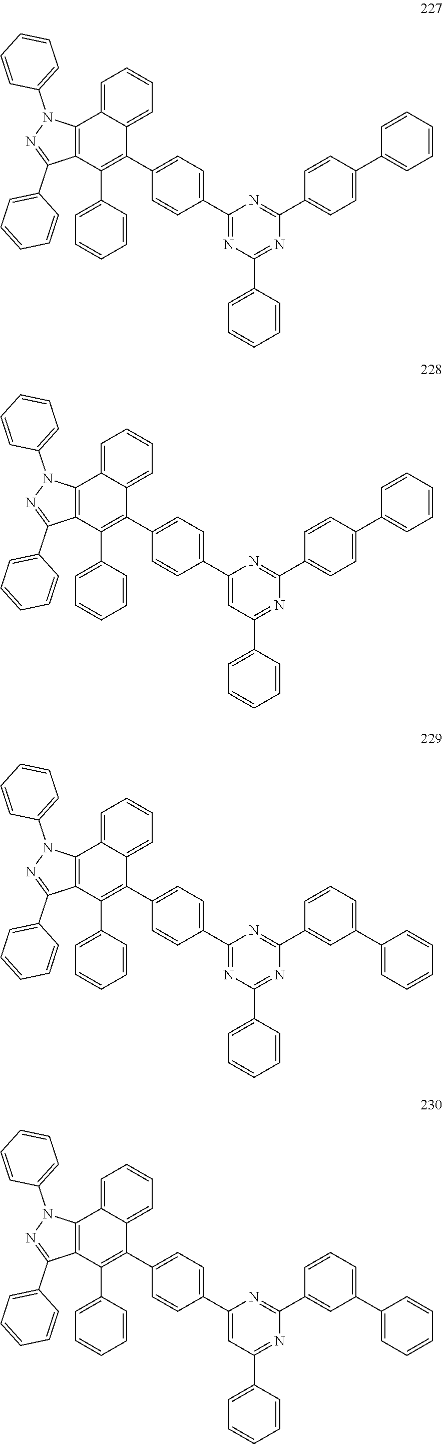 [SMILES: c1ccc(-c2ccc(-c3nc(-c4ccccc4)nc(-c4ccc(-c5c(-c6ccccc6)c6c(-c7ccccc7)nn(-c7ccccc7)c6c6ccccc56)cc4)n3)cc2)cc1 ; c1ccc(-c2ccc(-c3nc(-c4ccccc4)cc(-c4ccc(-c5c(-c6ccccc6)c6c(-c7ccccc7)nn(-c7ccccc7)c6c6ccccc56)cc4)n3)cc2)cc1 ; c1ccc(-c2cccc(-c3nc(-c4ccccc4)nc(-c4ccc(-c5c(-c6ccccc6)c6c(-c7ccccc7)nn(-c7ccccc7)c6c6ccccc56)cc4)n3)c2)cc1 ; c1ccc(-c2cccc(-c3nc(-c4ccccc4)cc(-c4ccc(-c5c(-c6ccccc6)c6c(-c7ccccc7)nn(-c7ccccc7)c6c6ccccc56)cc4)n3)c2)cc1]

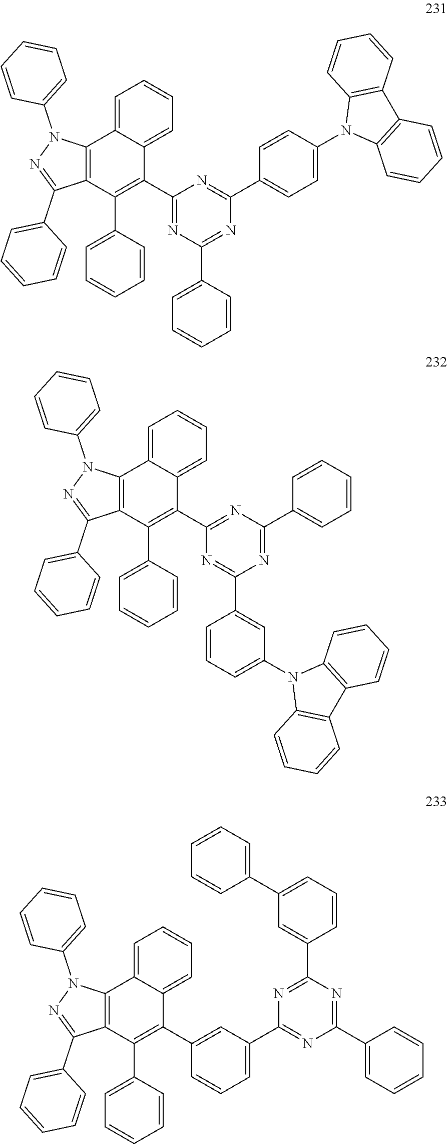 [SMILES: c1ccc(-c2nc(-c3ccc(-n4c5ccccc5c5ccccc54)cc3)nc(-c3c(-c4ccccc4)c4c(-c5ccccc5)nn(-c5ccccc5)c4c4ccccc34)n2)cc1 ; c1ccc(-c2nc(-c3cccc(-n4c5ccccc5c5ccccc54)c3)nc(-c3c(-c4ccccc4)c4c(-c5ccccc5)nn(-c5ccccc5)c4c4ccccc34)n2)cc1 ; c1ccc(-c2cccc(-c3nc(-c4ccccc4)nc(-c4cccc(-c5c(-c6ccccc6)c6c(-c7ccccc7)nn(-c7ccccc7)c6c6ccccc56)c4)n3)c2)cc1]

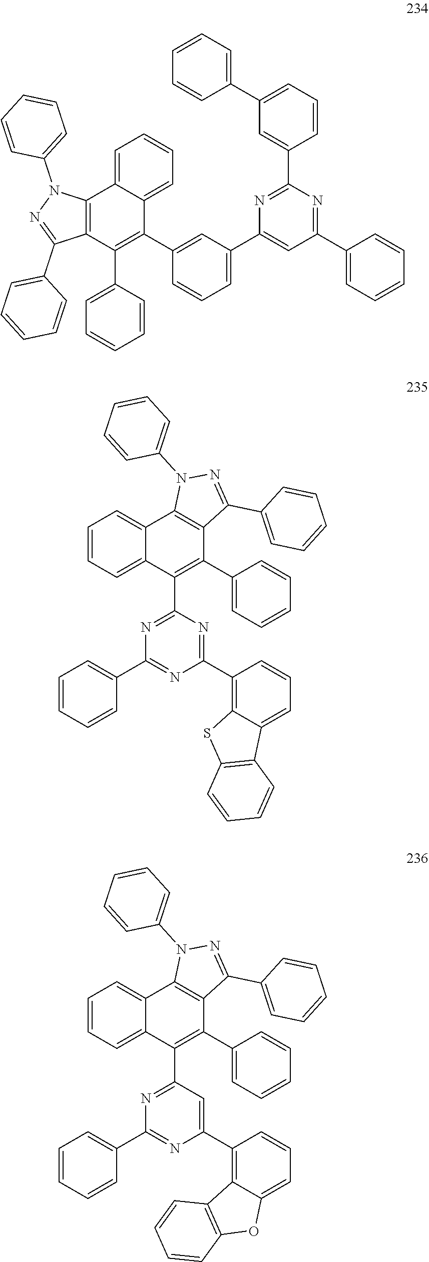 [SMILES: c1ccc(-c2cccc(-c3nc(-c4ccccc4)cc(-c4cccc(-c5c(-c6ccccc6)c6c(-c7ccccc7)nn(-c7ccccc7)c6c6ccccc56)c4)n3)c2)cc1 ; c1ccc(-c2nc(-c3c(-c4ccccc4)c4c(-c5ccccc5)nn(-c5ccccc5)c4c4ccccc34)nc(-c3cccc4c3sc3ccccc34)n2)cc1 ; c1ccc(-c2nc(-c3c(-c4ccccc4)c4c(-c5ccccc5)nn(-c5ccccc5)c4c4ccccc34)cc(-c3cccc4oc5ccccc5c34)n2)cc1]

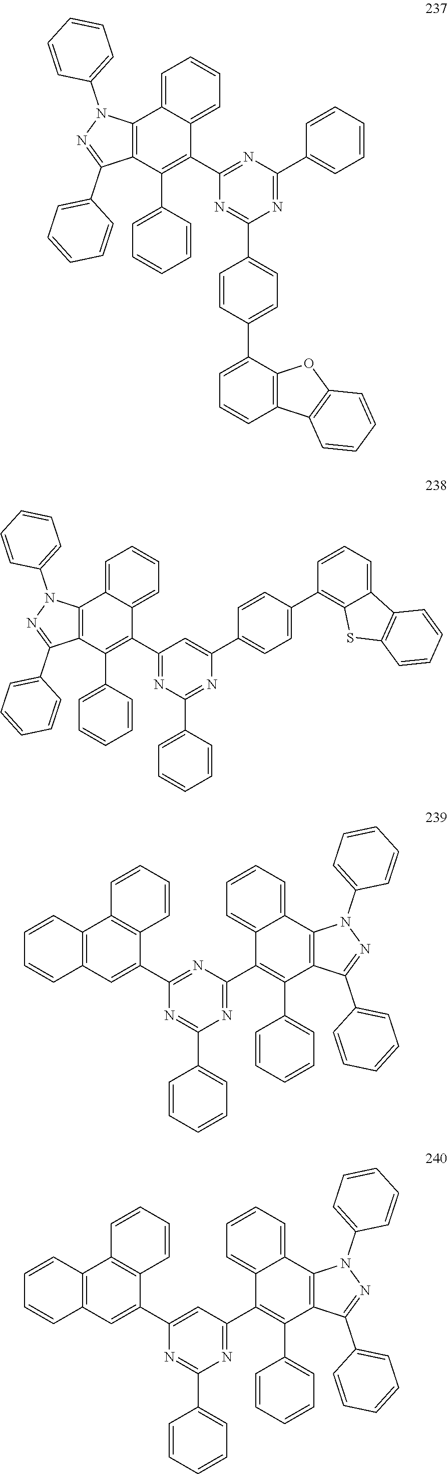 [SMILES: c1ccc(-c2nc(-c3ccc(-c4cccc5c4oc4ccccc45)cc3)nc(-c3c(-c4ccccc4)c4c(-c5ccccc5)nn(-c5ccccc5)c4c4ccccc34)n2)cc1 ; c1ccc(-c2nc(-c3ccc(-c4cccc5c4sc4ccccc45)cc3)cc(-c3c(-c4ccccc4)c4c(-c5ccccc5)nn(-c5ccccc5)c4c4ccccc34)n2)cc1 ; c1ccc(-c2nc(-c3cc4ccccc4c4ccccc34)nc(-c3c(-c4ccccc4)c4c(-c5ccccc5)nn(-c5ccccc5)c4c4ccccc34)n2)cc1 ; c1ccc(-c2nc(-c3cc4ccccc4c4ccccc34)cc(-c3c(-c4ccccc4)c4c(-c5ccccc5)nn(-c5ccccc5)c4c4ccccc34)n2)cc1]

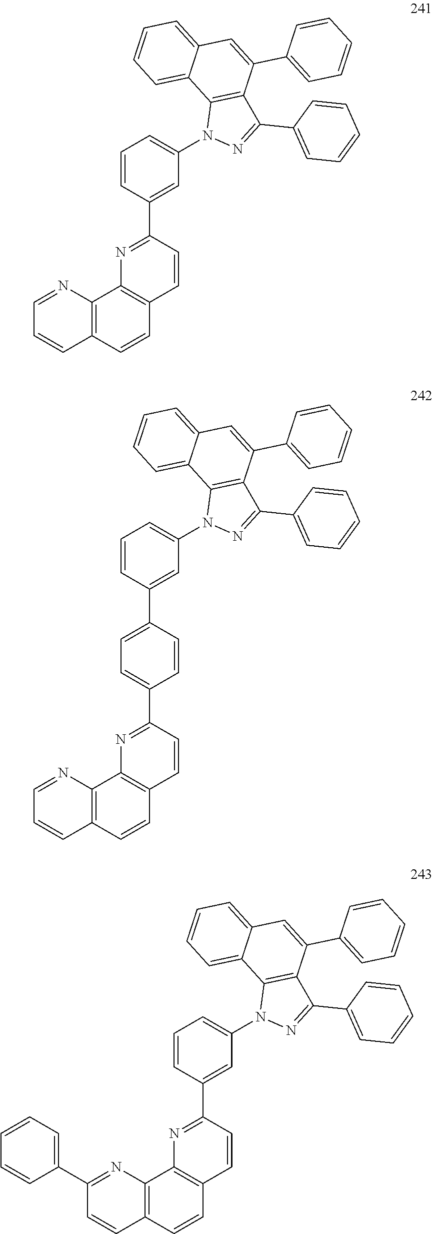 [SMILES: c1ccc(-c2cc3ccccc3c3c2c(-c2ccccc2)nn3-c2cccc(-c3ccc4ccc5cccnc5c4n3)c2)cc1 ; c1ccc(-c2cc3ccccc3c3c2c(-c2ccccc2)nn3-c2cccc(-c3ccc(-c4ccc5ccc6cccnc6c5n4)cc3)c2)cc1 ; c1ccc(-c2ccc3ccc4ccc(-c5cccc(-n6nc(-c7ccccc7)c7c(-c8ccccc8)cc8ccccc8c76)c5)nc4c3n2)cc1]

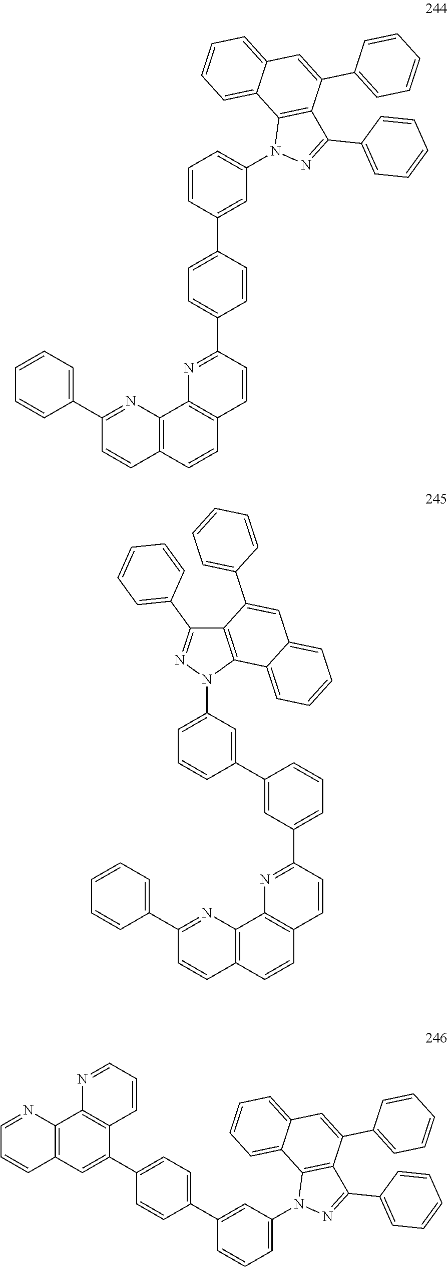 [SMILES: c1ccc(-c2ccc3ccc4ccc(-c5ccc(-c6cccc(-n7nc(-c8ccccc8)c8c(-c9ccccc9)cc9ccccc9c87)c6)cc5)nc4c3n2)cc1 ; c1ccc(-c2ccc3ccc4ccc(-c5cccc(-c6cccc(-n7nc(-c8ccccc8)c8c(-c9ccccc9)cc9ccccc9c87)c6)c5)nc4c3n2)cc1 ; c1ccc(-c2cc3ccccc3c3c2c(-c2ccccc2)nn3-c2cccc(-c3ccc(-c4cc5cccnc5c5ncccc45)cc3)c2)cc1]

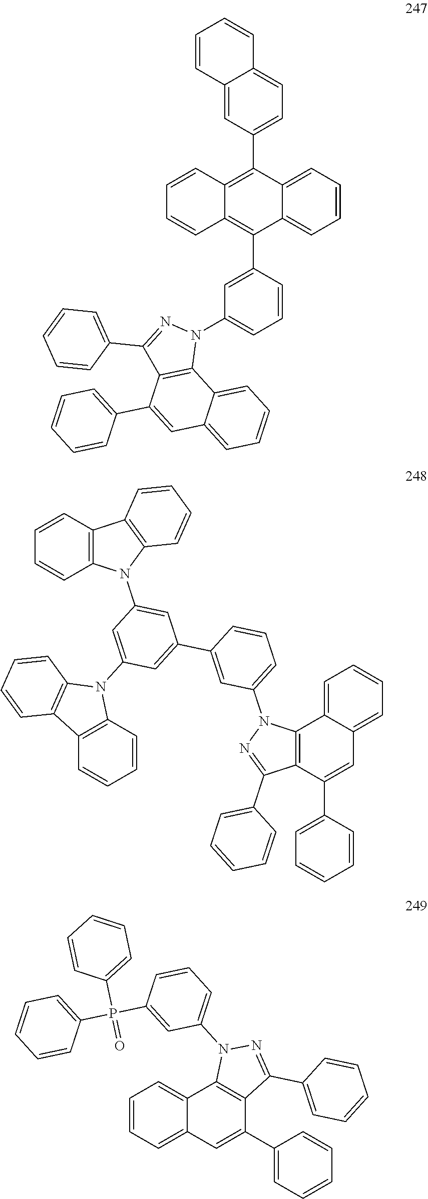 [SMILES: c1ccc(-c2cc3ccccc3c3c2c(-c2ccccc2)nn3-c2cccc(-c3c4ccccc4c(-c4ccc5ccccc5c4)c4ccccc34)c2)cc1 ; c1ccc(-c2cc3ccccc3c3c2c(-c2ccccc2)nn3-c2cccc(-c3cc(-n4c5ccccc5c5ccccc54)cc(-n4c5ccccc5c5ccccc54)c3)c2)cc1 ; O=P(c1ccccc1)(c1ccccc1)c1cccc(-n2nc(-c3ccccc3)c3c(-c4ccccc4)cc4ccccc4c32)c1]

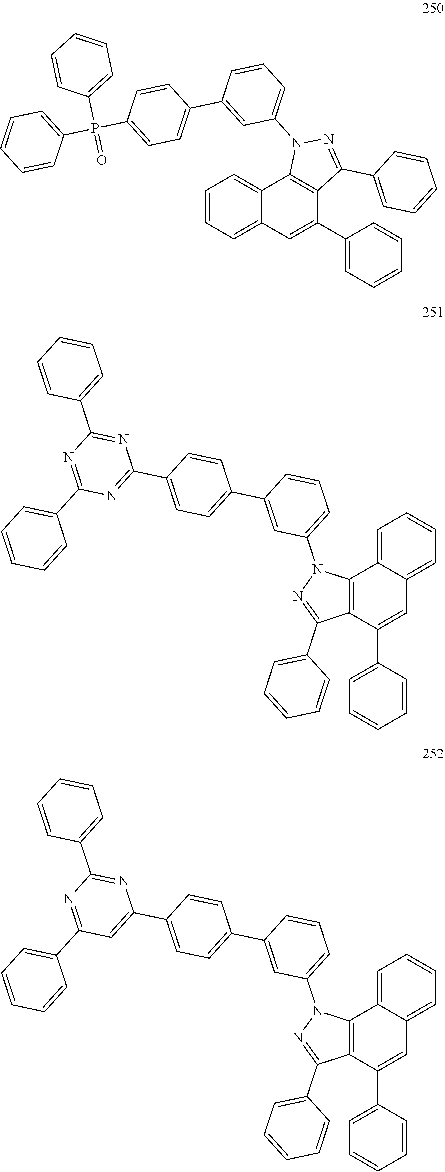 [SMILES: O=P(c1ccccc1)(c1ccccc1)c1ccc(-c2cccc(-n3nc(-c4ccccc4)c4c(-c5ccccc5)cc5ccccc5c43)c2)cc1 ; c1ccc(-c2nc(-c3ccccc3)nc(-c3ccc(-c4cccc(-n5nc(-c6ccccc6)c6c(-c7ccccc7)cc7ccccc7c65)c4)cc3)n2)cc1 ; c1ccc(-c2cc(-c3ccc(-c4cccc(-n5nc(-c6ccccc6)c6c(-c7ccccc7)cc7ccccc7c65)c4)cc3)nc(-c3ccccc3)n2)cc1]

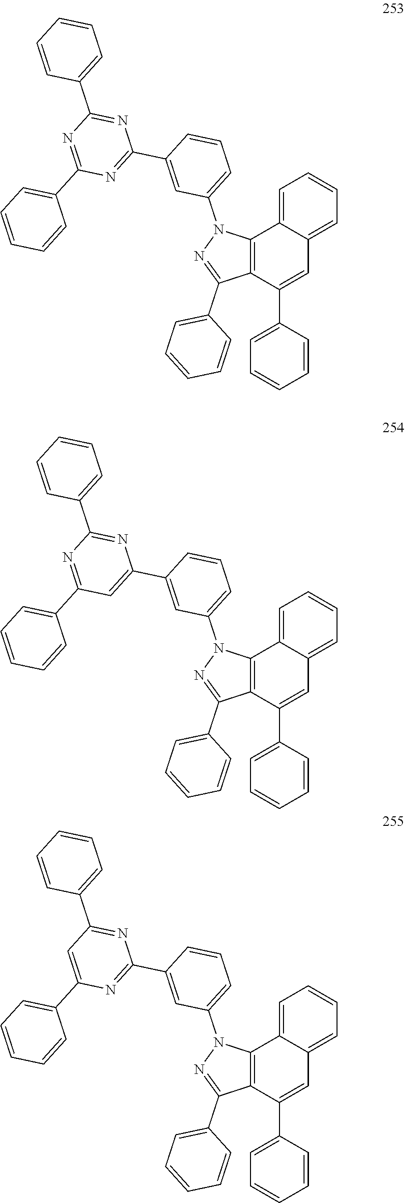 [SMILES: c1ccc(-c2nc(-c3ccccc3)nc(-c3cccc(-n4nc(-c5ccccc5)c5c(-c6ccccc6)cc6ccccc6c54)c3)n2)cc1 ; c1ccc(-c2cc(-c3cccc(-n4nc(-c5ccccc5)c5c(-c6ccccc6)cc6ccccc6c54)c3)nc(-c3ccccc3)n2)cc1 ; c1ccc(-c2cc(-c3ccccc3)nc(-c3cccc(-n4nc(-c5ccccc5)c5c(-c6ccccc6)cc6ccccc6c54)c3)n2)cc1]

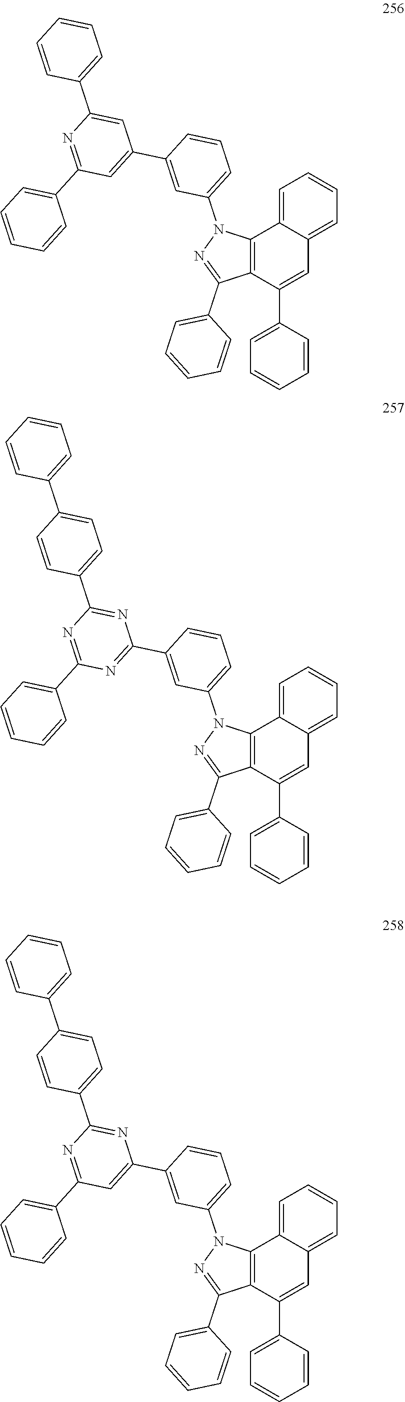 [SMILES: c1ccc(-c2cc(-c3cccc(-n4nc(-c5ccccc5)c5c(-c6ccccc6)cc6ccccc6c54)c3)cc(-c3ccccc3)n2)cc1 ; c1ccc(-c2ccc(-c3nc(-c4ccccc4)nc(-c4cccc(-n5nc(-c6ccccc6)c6c(-c7ccccc7)cc7ccccc7c65)c4)n3)cc2)cc1 ; c1ccc(-c2ccc(-c3nc(-c4ccccc4)cc(-c4cccc(-n5nc(-c6ccccc6)c6c(-c7ccccc7)cc7ccccc7c65)c4)n3)cc2)cc1]

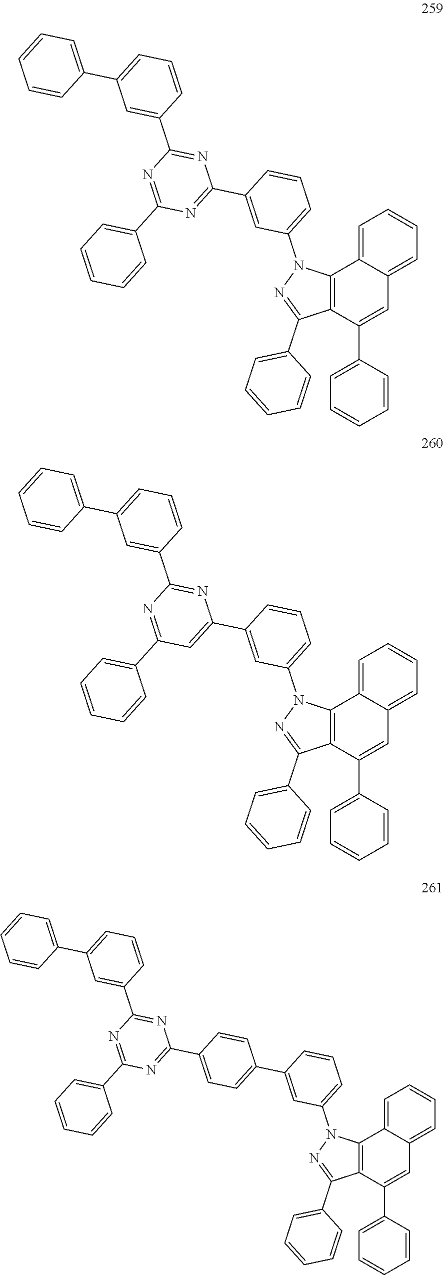 [SMILES: c1ccc(-c2cccc(-c3nc(-c4ccccc4)nc(-c4cccc(-n5nc(-c6ccccc6)c6c(-c7ccccc7)cc7ccccc7c65)c4)n3)c2)cc1 ; c1ccc(-c2cccc(-c3nc(-c4ccccc4)cc(-c4cccc(-n5nc(-c6ccccc6)c6c(-c7ccccc7)cc7ccccc7c65)c4)n3)c2)cc1 ; c1ccc(-c2cccc(-c3nc(-c4ccccc4)nc(-c4ccc(-c5cccc(-n6nc(-c7ccccc7)c7c(-c8ccccc8)cc8ccccc8c76)c5)cc4)n3)c2)cc1]

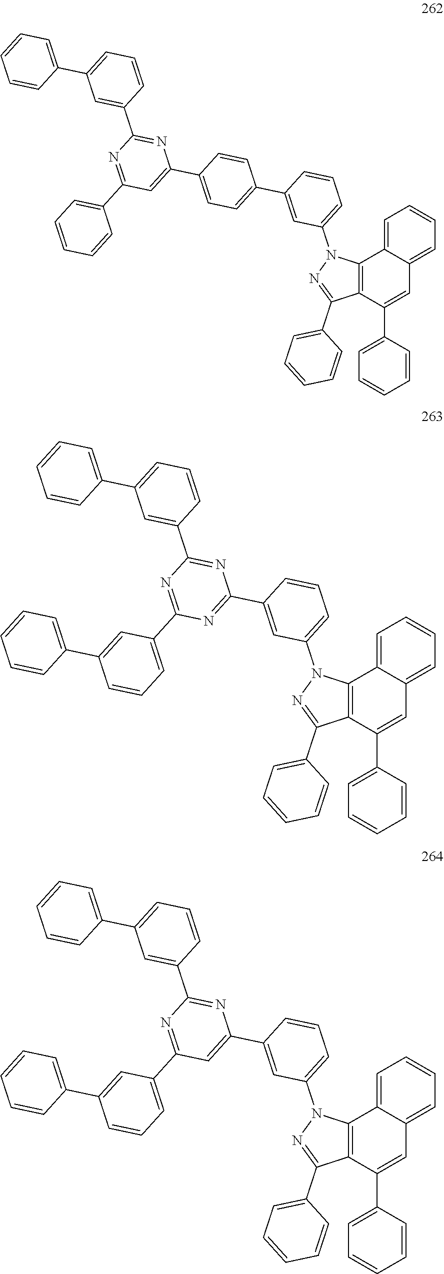 [SMILES: c1ccc(-c2cccc(-c3nc(-c4ccccc4)cc(-c4ccc(-c5cccc(-n6nc(-c7ccccc7)c7c(-c8ccccc8)cc8ccccc8c76)c5)cc4)n3)c2)cc1 ; c1ccc(-c2cccc(-c3nc(-c4cccc(-c5ccccc5)c4)nc(-c4cccc(-n5nc(-c6ccccc6)c6c(-c7ccccc7)cc7ccccc7c65)c4)n3)c2)cc1 ; c1ccc(-c2cccc(-c3cc(-c4cccc(-n5nc(-c6ccccc6)c6c(-c7ccccc7)cc7ccccc7c65)c4)nc(-c4cccc(-c5ccccc5)c4)n3)c2)cc1]

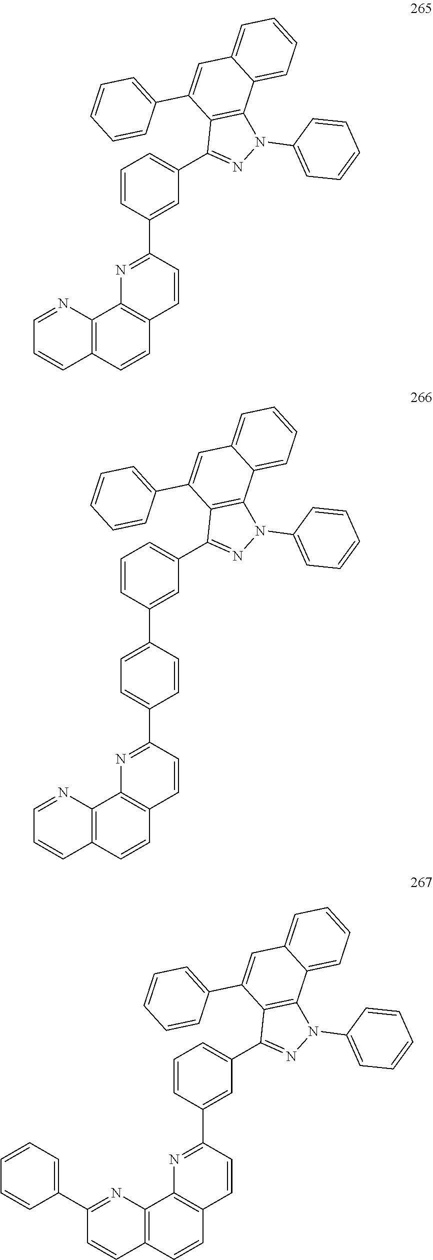 [SMILES: c1ccc(-c2cc3ccccc3c3c2c(-c2cccc(-c4ccc5ccc6cccnc6c5n4)c2)nn3-c2ccccc2)cc1 ; c1ccc(-c2cc3ccccc3c3c2c(-c2cccc(-c4ccc(-c5ccc6ccc7cccnc7c6n5)cc4)c2)nn3-c2ccccc2)cc1 ; c1ccc(-c2ccc3ccc4ccc(-c5cccc(-c6nn(-c7ccccc7)c7c6c(-c6ccccc6)cc6ccccc67)c5)nc4c3n2)cc1]

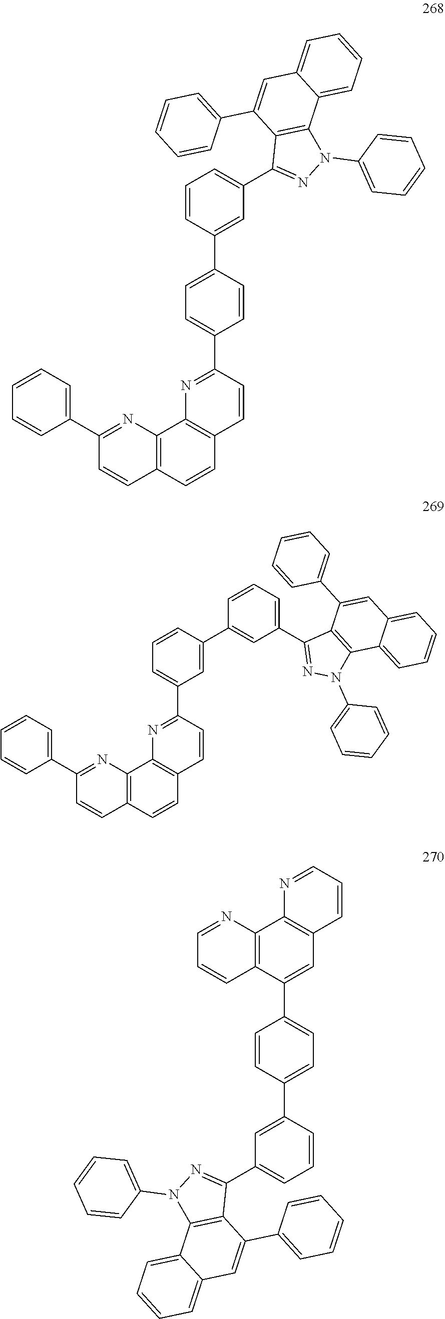 [SMILES: c1ccc(-c2ccc3ccc4ccc(-c5ccc(-c6cccc(-c7nn(-c8ccccc8)c8c7c(-c7ccccc7)cc7ccccc78)c6)cc5)nc4c3n2)cc1 ; c1ccc(-c2ccc3ccc4ccc(-c5cccc(-c6cccc(-c7nn(-c8ccccc8)c8c7c(-c7ccccc7)cc7ccccc78)c6)c5)nc4c3n2)cc1 ; c1ccc(-c2cc3ccccc3c3c2c(-c2cccc(-c4ccc(-c5cc6cccnc6c6ncccc56)cc4)c2)nn3-c2ccccc2)cc1]

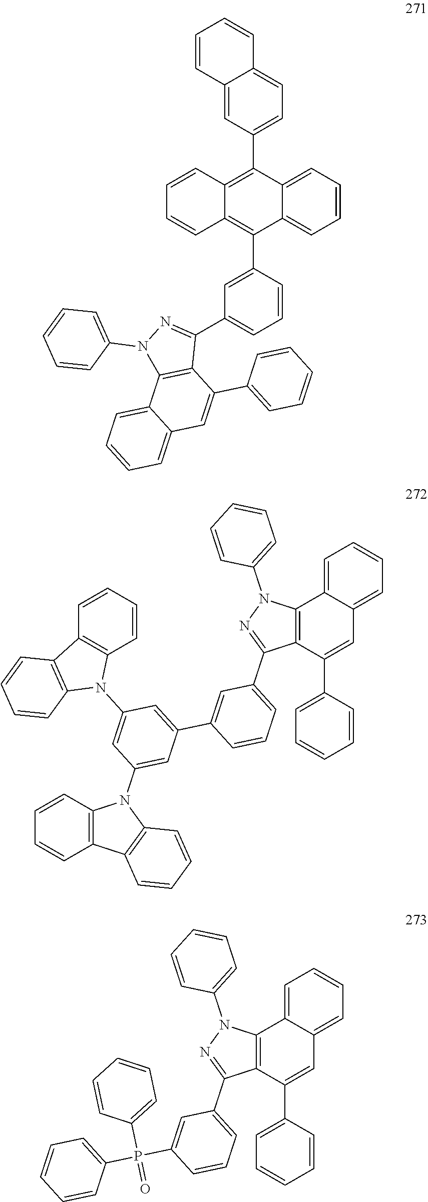 [SMILES: c1ccc(-c2cc3ccccc3c3c2c(-c2cccc(-c4c5ccccc5c(-c5ccc6ccccc6c5)c5ccccc45)c2)nn3-c2ccccc2)cc1 ; c1ccc(-c2cc3ccccc3c3c2c(-c2cccc(-c4cc(-n5c6ccccc6c6ccccc65)cc(-n5c6ccccc6c6ccccc65)c4)c2)nn3-c2ccccc2)cc1 ; O=P(c1ccccc1)(c1ccccc1)c1cccc(-c2nn(-c3ccccc3)c3c2c(-c2ccccc2)cc2ccccc23)c1]

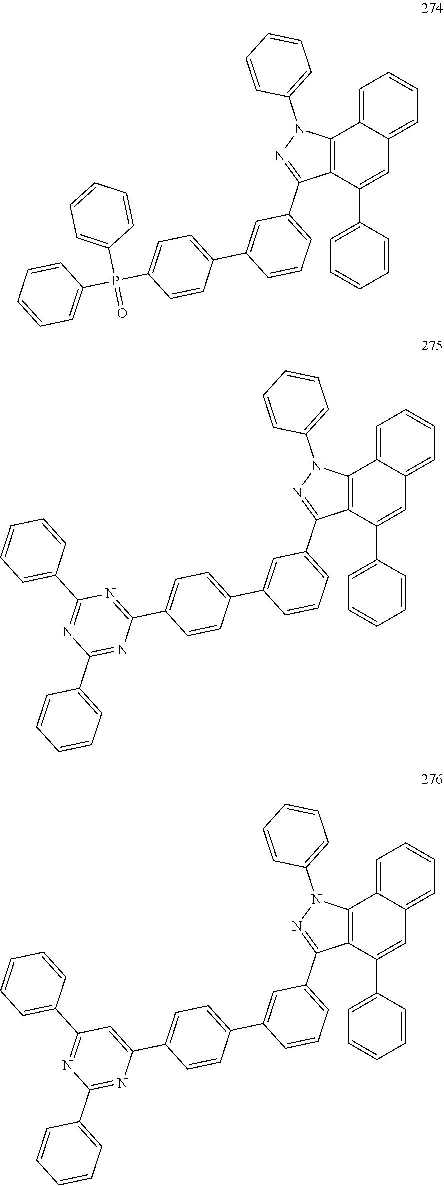 [SMILES: O=P(c1ccccc1)(c1ccccc1)c1ccc(-c2cccc(-c3nn(-c4ccccc4)c4c3c(-c3ccccc3)cc3ccccc34)c2)cc1 ; c1ccc(-c2nc(-c3ccccc3)nc(-c3ccc(-c4cccc(-c5nn(-c6ccccc6)c6c5c(-c5ccccc5)cc5ccccc56)c4)cc3)n2)cc1 ; c1ccc(-c2cc(-c3ccc(-c4cccc(-c5nn(-c6ccccc6)c6c5c(-c5ccccc5)cc5ccccc56)c4)cc3)nc(-c3ccccc3)n2)cc1]

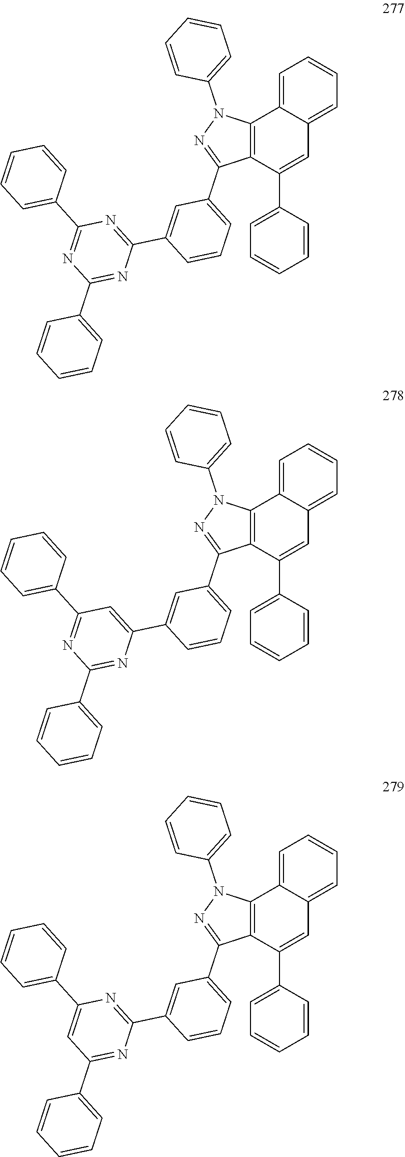 [SMILES: c1ccc(-c2nc(-c3ccccc3)nc(-c3cccc(-c4nn(-c5ccccc5)c5c4c(-c4ccccc4)cc4ccccc45)c3)n2)cc1 ; c1ccc(-c2cc(-c3cccc(-c4nn(-c5ccccc5)c5c4c(-c4ccccc4)cc4ccccc45)c3)nc(-c3ccccc3)n2)cc1 ; c1ccc(-c2cc(-c3ccccc3)nc(-c3cccc(-c4nn(-c5ccccc5)c5c4c(-c4ccccc4)cc4ccccc45)c3)n2)cc1]

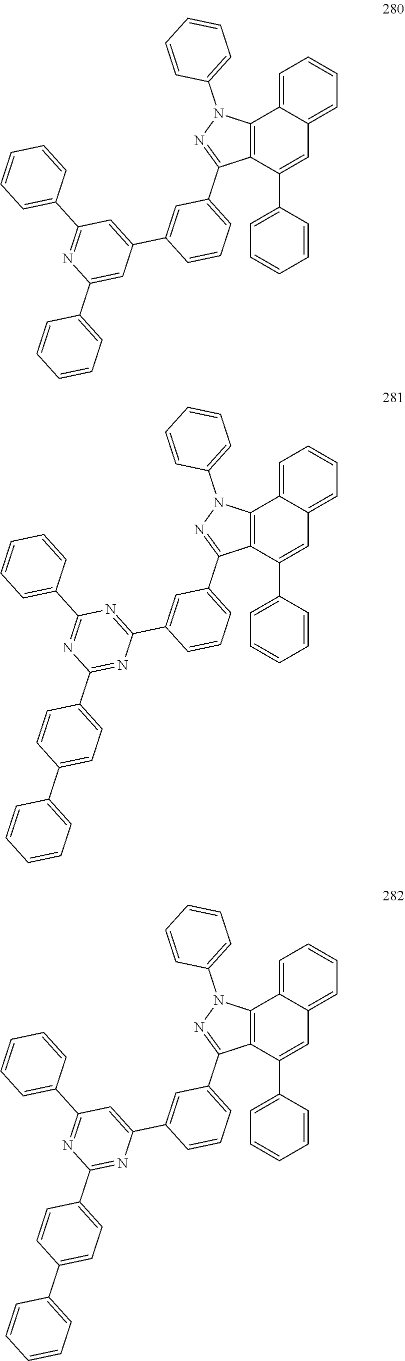 [SMILES: c1ccc(-c2cc(-c3cccc(-c4nn(-c5ccccc5)c5c4c(-c4ccccc4)cc4ccccc45)c3)cc(-c3ccccc3)n2)cc1 ; c1ccc(-c2ccc(-c3nc(-c4ccccc4)nc(-c4cccc(-c5nn(-c6ccccc6)c6c5c(-c5ccccc5)cc5ccccc56)c4)n3)cc2)cc1 ; c1ccc(-c2ccc(-c3nc(-c4ccccc4)cc(-c4cccc(-c5nn(-c6ccccc6)c6c5c(-c5ccccc5)cc5ccccc56)c4)n3)cc2)cc1]

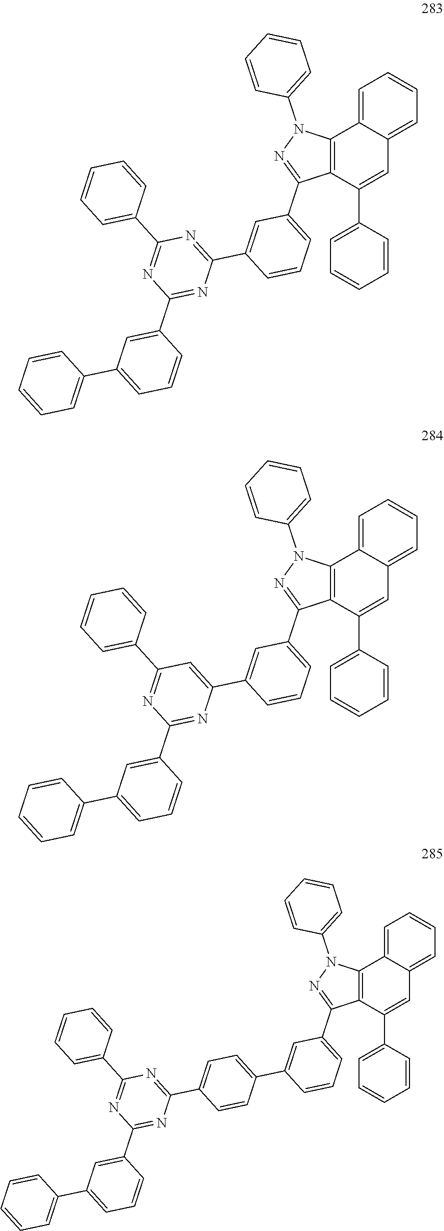 [SMILES: c1ccc(-c2cccc(-c3nc(-c4ccccc4)nc(-c4cccc(-c5nn(-c6ccccc6)c6c5c(-c5ccccc5)cc5ccccc56)c4)n3)c2)cc1 ; c1ccc(-c2cccc(-c3nc(-c4ccccc4)cc(-c4cccc(-c5nn(-c6ccccc6)c6c5c(-c5ccccc5)cc5ccccc56)c4)n3)c2)cc1 ; c1ccc(-c2cccc(-c3nc(-c4ccccc4)nc(-c4ccc(-c5cccc(-c6nn(-c7ccccc7)c7c6c(-c6ccccc6)cc6ccccc67)c5)cc4)n3)c2)cc1]

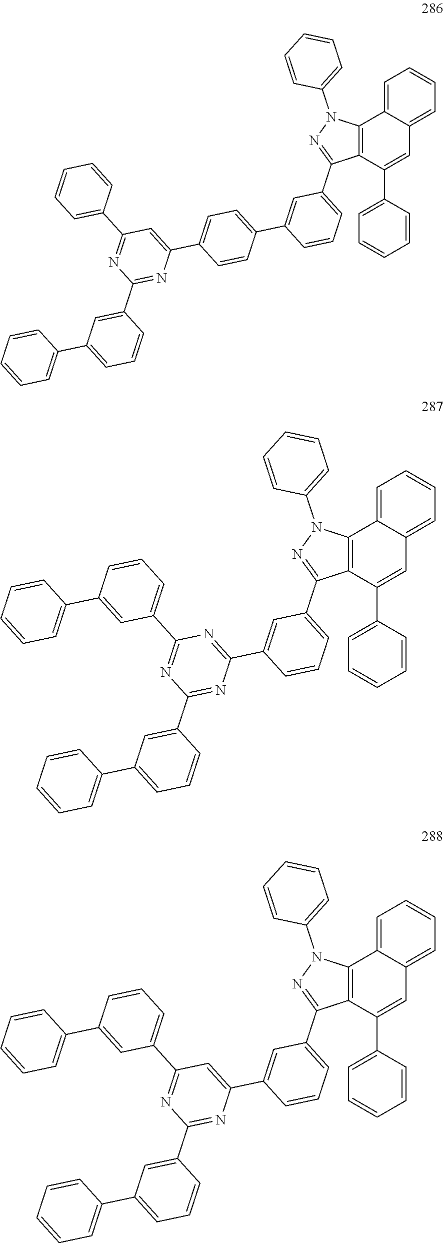 [SMILES: c1ccc(-c2cccc(-c3nc(-c4ccccc4)cc(-c4ccc(-c5cccc(-c6nn(-c7ccccc7)c7c6c(-c6ccccc6)cc6ccccc67)c5)cc4)n3)c2)cc1 ; c1ccc(-c2cccc(-c3nc(-c4cccc(-c5ccccc5)c4)nc(-c4cccc(-c5nn(-c6ccccc6)c6c5c(-c5ccccc5)cc5ccccc56)c4)n3)c2)cc1 ; c1ccc(-c2cccc(-c3cc(-c4cccc(-c5nn(-c6ccccc6)c6c5c(-c5ccccc5)cc5ccccc56)c4)nc(-c4cccc(-c5ccccc5)c4)n3)c2)cc1]

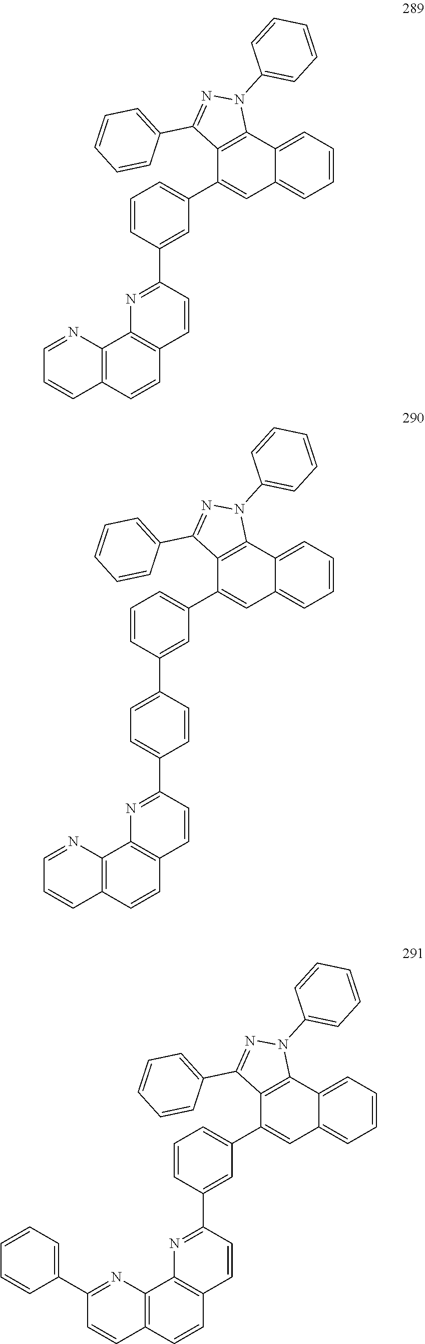 [SMILES: c1ccc(-c2nn(-c3ccccc3)c3c2c(-c2cccc(-c4ccc5ccc6cccnc6c5n4)c2)cc2ccccc23)cc1 ; c1ccc(-c2nn(-c3ccccc3)c3c2c(-c2cccc(-c4ccc(-c5ccc6ccc7cccnc7c6n5)cc4)c2)cc2ccccc23)cc1 ; c1ccc(-c2ccc3ccc4ccc(-c5cccc(-c6cc7ccccc7c7c6c(-c6ccccc6)nn7-c6ccccc6)c5)nc4c3n2)cc1]

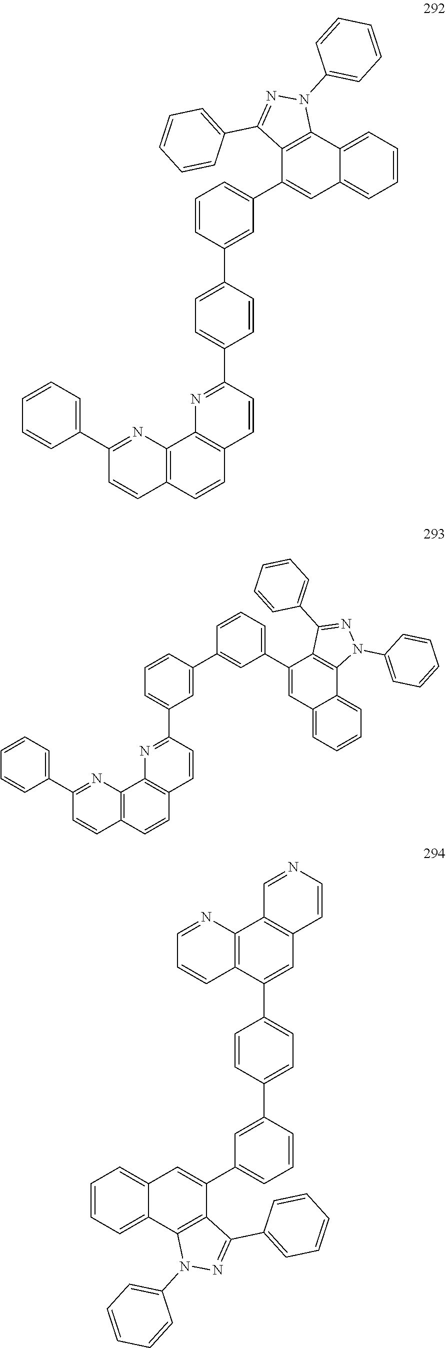 [SMILES: c1ccc(-c2ccc3ccc4ccc(-c5ccc(-c6cccc(-c7cc8ccccc8c8c7c(-c7ccccc7)nn8-c7ccccc7)c6)cc5)nc4c3n2)cc1 ; c1ccc(-c2ccc3ccc4ccc(-c5cccc(-c6cccc(-c7cc8ccccc8c8c7c(-c7ccccc7)nn8-c7ccccc7)c6)c5)nc4c3n2)cc1 ; c1ccc(-c2nn(-c3ccccc3)c3c2c(-c2cccc(-c4ccc(-c5cc6ccncc6c6ncccc56)cc4)c2)cc2ccccc23)cc1]

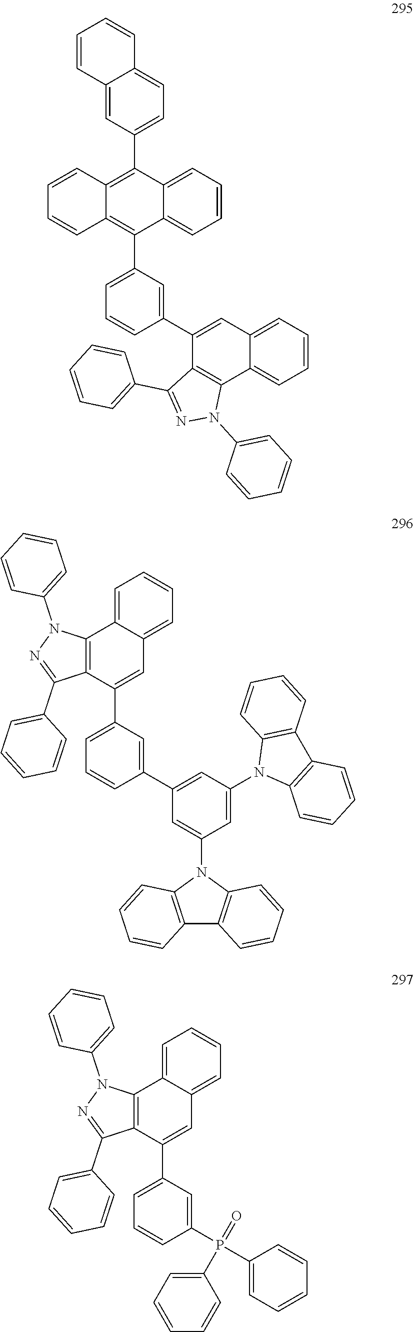 [SMILES: c1ccc(-c2nn(-c3ccccc3)c3c2c(-c2cccc(-c4c5ccccc5c(-c5ccc6ccccc6c5)c5ccccc45)c2)cc2ccccc23)cc1 ; c1ccc(-c2nn(-c3ccccc3)c3c2c(-c2cccc(-c4cc(-n5c6ccccc6c6ccccc65)cc(-n5c6ccccc6c6ccccc65)c4)c2)cc2ccccc23)cc1 ; O=P(c1ccccc1)(c1ccccc1)c1cccc(-c2cc3ccccc3c3c2c(-c2ccccc2)nn3-c2ccccc2)c1]

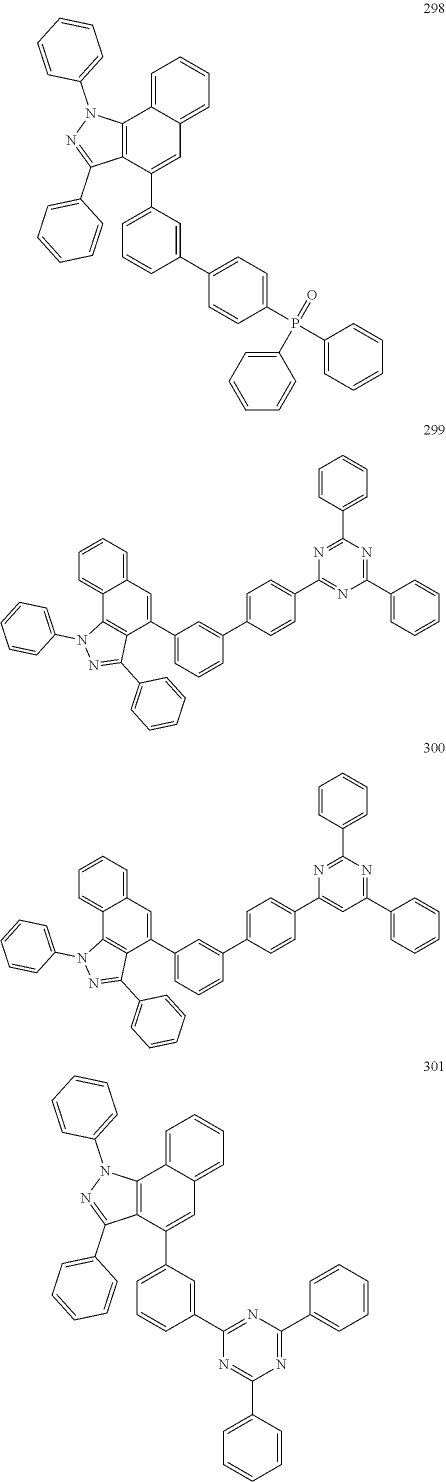 [SMILES: O=P(c1ccccc1)(c1ccccc1)c1ccc(-c2cccc(-c3cc4ccccc4c4c3c(-c3ccccc3)nn4-c3ccccc3)c2)cc1 ; c1ccc(-c2nc(-c3ccccc3)nc(-c3ccc(-c4cccc(-c5cc6ccccc6c6c5c(-c5ccccc5)nn6-c5ccccc5)c4)cc3)n2)cc1 ; c1ccc(-c2cc(-c3ccc(-c4cccc(-c5cc6ccccc6c6c5c(-c5ccccc5)nn6-c5ccccc5)c4)cc3)nc(-c3ccccc3)n2)cc1 ; c1ccc(-c2nc(-c3ccccc3)nc(-c3cccc(-c4cc5ccccc5c5c4c(-c4ccccc4)nn5-c4ccccc4)c3)n2)cc1]

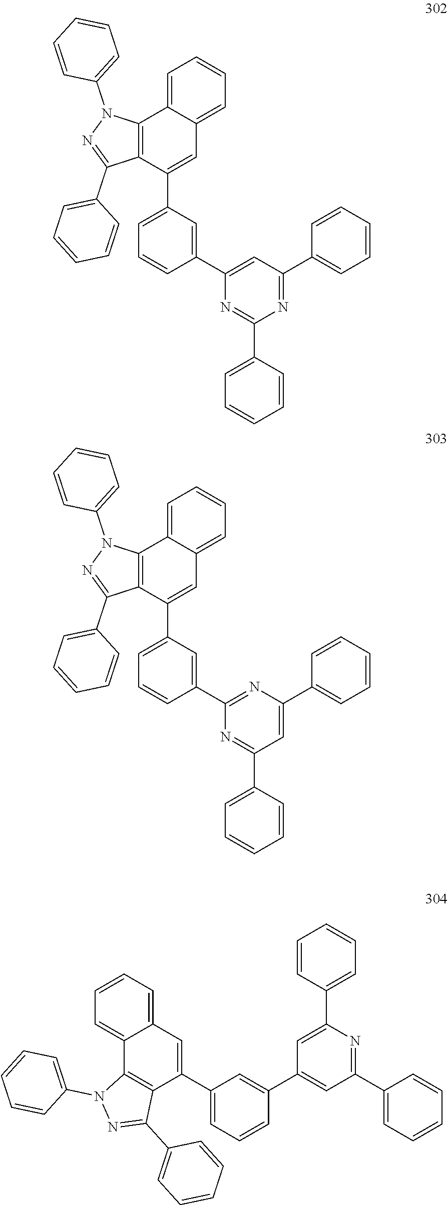 [SMILES: c1ccc(-c2cc(-c3cccc(-c4cc5ccccc5c5c4c(-c4ccccc4)nn5-c4ccccc4)c3)nc(-c3ccccc3)n2)cc1 ; c1ccc(-c2cc(-c3ccccc3)nc(-c3cccc(-c4cc5ccccc5c5c4c(-c4ccccc4)nn5-c4ccccc4)c3)n2)cc1 ; c1ccc(-c2cc(-c3cccc(-c4cc5ccccc5c5c4c(-c4ccccc4)nn5-c4ccccc4)c3)cc(-c3ccccc3)n2)cc1]

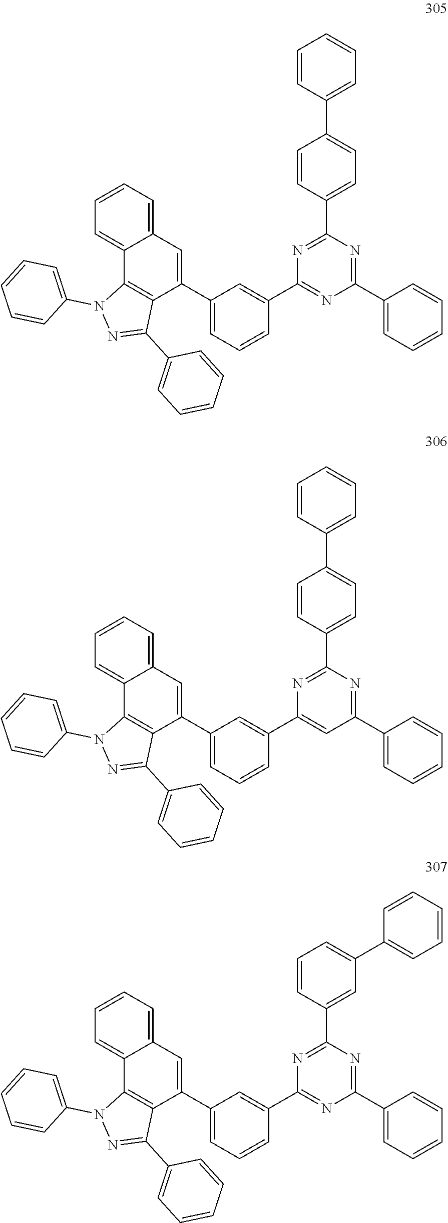 [SMILES: c1ccc(-c2ccc(-c3nc(-c4ccccc4)nc(-c4cccc(-c5cc6ccccc6c6c5c(-c5ccccc5)nn6-c5ccccc5)c4)n3)cc2)cc1 ; c1ccc(-c2ccc(-c3nc(-c4ccccc4)cc(-c4cccc(-c5cc6ccccc6c6c5c(-c5ccccc5)nn6-c5ccccc5)c4)n3)cc2)cc1 ; c1ccc(-c2cccc(-c3nc(-c4ccccc4)nc(-c4cccc(-c5cc6ccccc6c6c5c(-c5ccccc5)nn6-c5ccccc5)c4)n3)c2)cc1]

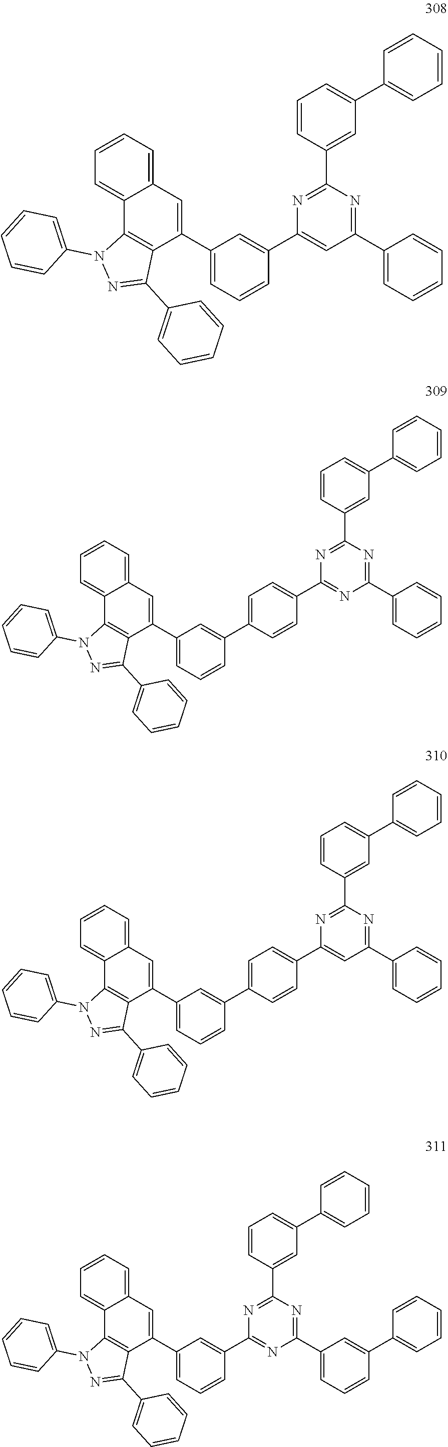 [SMILES: c1ccc(-c2cccc(-c3nc(-c4ccccc4)cc(-c4cccc(-c5cc6ccccc6c6c5c(-c5ccccc5)nn6-c5ccccc5)c4)n3)c2)cc1 ; c1ccc(-c2cccc(-c3nc(-c4ccccc4)nc(-c4ccc(-c5cccc(-c6cc7ccccc7c7c6c(-c6ccccc6)nn7-c6ccccc6)c5)cc4)n3)c2)cc1 ; c1ccc(-c2cccc(-c3nc(-c4ccccc4)cc(-c4ccc(-c5cccc(-c6cc7ccccc7c7c6c(-c6ccccc6)nn7-c6ccccc6)c5)cc4)n3)c2)cc1 ; c1ccc(-c2cccc(-c3nc(-c4cccc(-c5ccccc5)c4)nc(-c4cccc(-c5cc6ccccc6c6c5c(-c5ccccc5)nn6-c5ccccc5)c4)n3)c2)cc1]

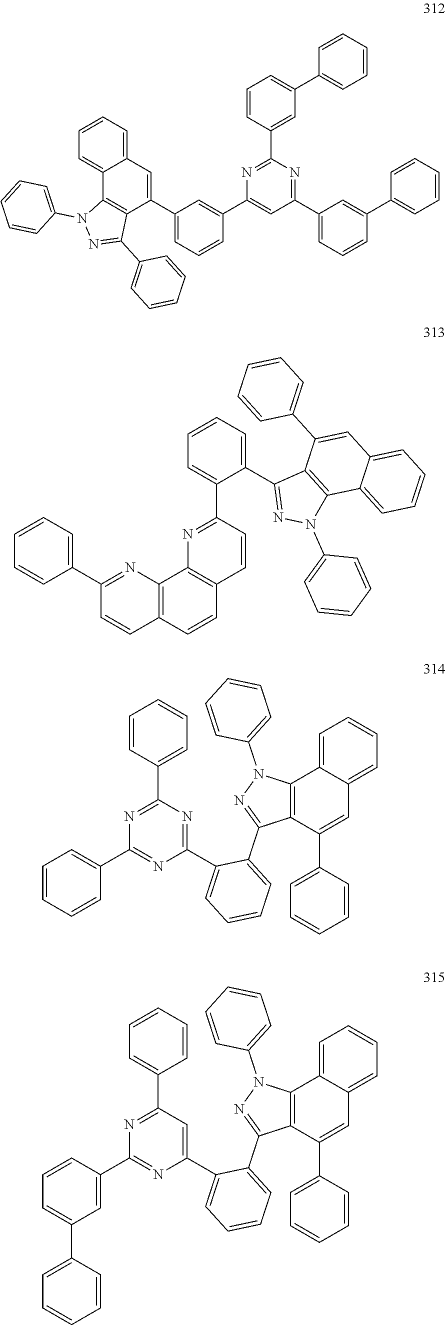 [SMILES: c1ccc(-c2cccc(-c3cc(-c4cccc(-c5cc6ccccc6c6c5c(-c5ccccc5)nn6-c5ccccc5)c4)nc(-c4cccc(-c5ccccc5)c4)n3)c2)cc1 ; c1ccc(-c2ccc3ccc4ccc(-c5ccccc5-c5nn(-c6ccccc6)c6c5c(-c5ccccc5)cc5ccccc56)nc4c3n2)cc1 ; c1ccc(-c2nc(-c3ccccc3)nc(-c3ccccc3-c3nn(-c4ccccc4)c4c3c(-c3ccccc3)cc3ccccc34)n2)cc1 ; c1ccc(-c2cccc(-c3nc(-c4ccccc4)cc(-c4ccccc4-c4nn(-c5ccccc5)c5c4c(-c4ccccc4)cc4ccccc45)n3)c2)cc1]

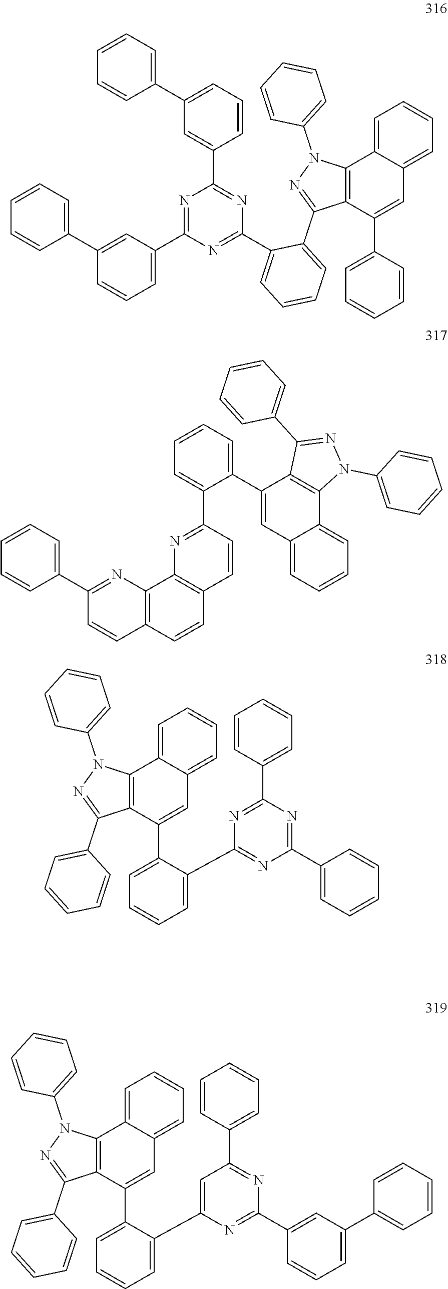

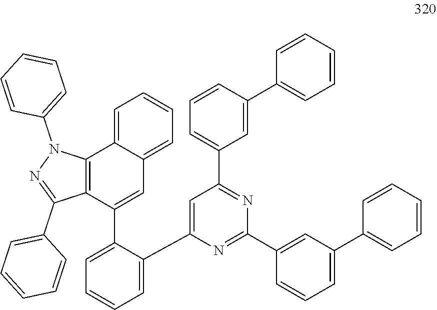 [SMILES: c1ccc(-c2cccc(-c3cc(-c4ccccc4-c4cc5ccccc5c5c4c(-c4ccccc4)nn5-c4ccccc4)nc(-c4cccc(-c5ccccc5)c4)n3)c2)cc1]

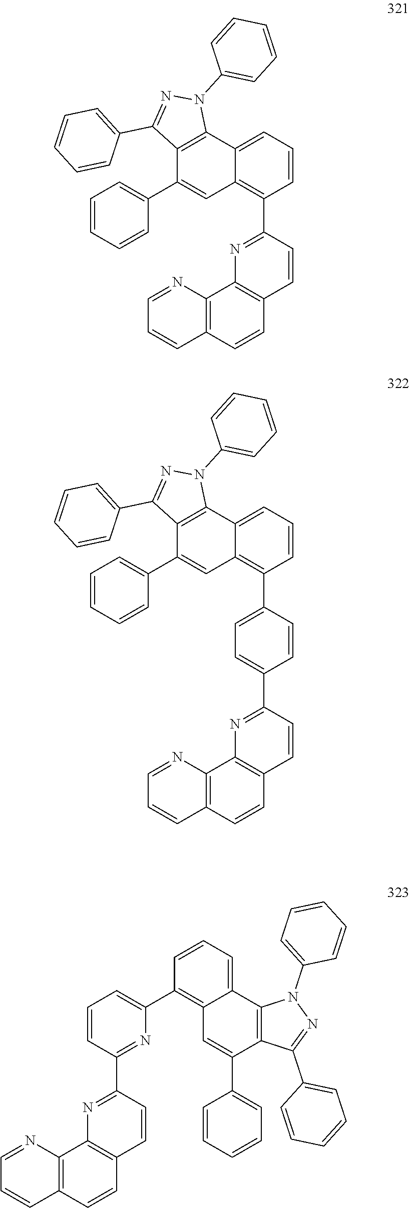

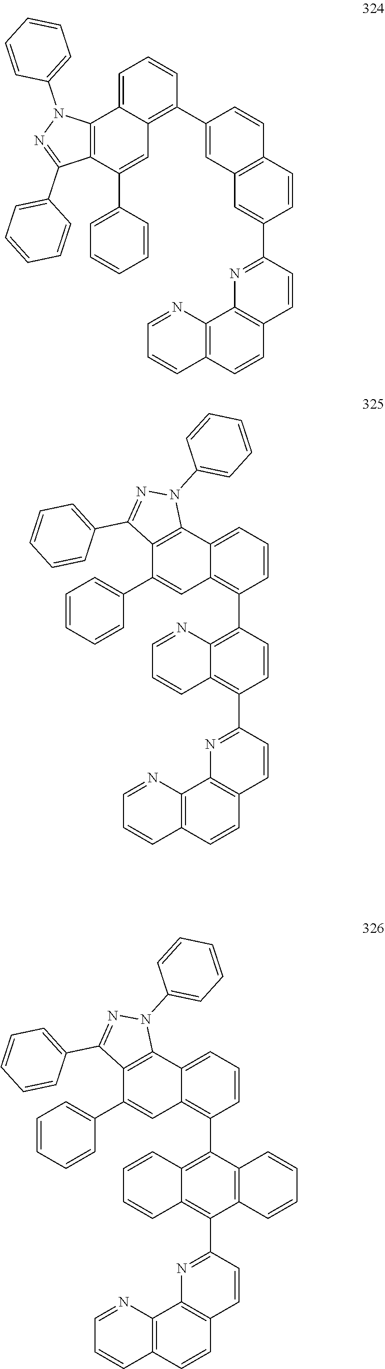 [SMILES: c1ccc(-c2cc3c(-c4ccc5ccc(-c6ccc7ccc8cccnc8c7n6)cc5c4)cccc3c3c2c(-c2ccccc2)nn3-c2ccccc2)cc1 ; c1ccc(-c2cc3c(-c4ccc(-c5ccc6ccc7cccnc7c6n5)c5cccnc45)cccc3c3c2c(-c2ccccc2)nn3-c2ccccc2)cc1 ; c1ccc(-c2cc3c(-c4c5ccccc5c(-c5ccc6ccc7cccnc7c6n5)c5ccccc45)cccc3c3c2c(-c2ccccc2)nn3-c2ccccc2)cc1]

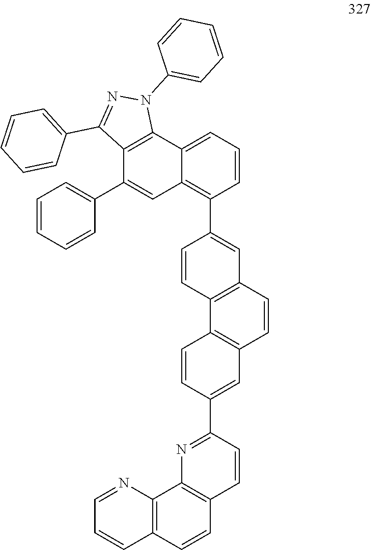 [SMILES: c1ccc(-c2cc3c(-c4ccc5c(ccc6cc(-c7ccc8ccc9cccnc9c8n7)ccc65)c4)cccc3c3c2c(-c2ccccc2)nn3-c2ccccc2)cc1]

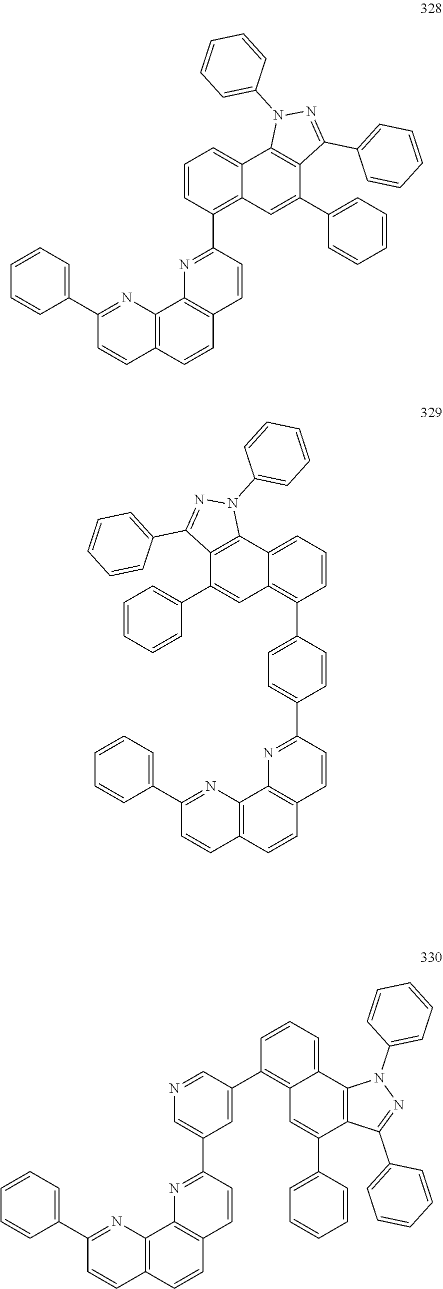 [SMILES: c1ccc(-c2ccc3ccc4ccc(-c5cccc6c5cc(-c5ccccc5)c5c(-c7ccccc7)nn(-c7ccccc7)c56)nc4c3n2)cc1 ; c1ccc(-c2ccc3ccc4ccc(-c5ccc(-c6cccc7c6cc(-c6ccccc6)c6c(-c8ccccc8)nn(-c8ccccc8)c67)cc5)nc4c3n2)cc1 ; c1ccc(-c2ccc3ccc4ccc(-c5cncc(-c6cccc7c6cc(-c6ccccc6)c6c(-c8ccccc8)nn(-c8ccccc8)c67)c5)nc4c3n2)cc1]

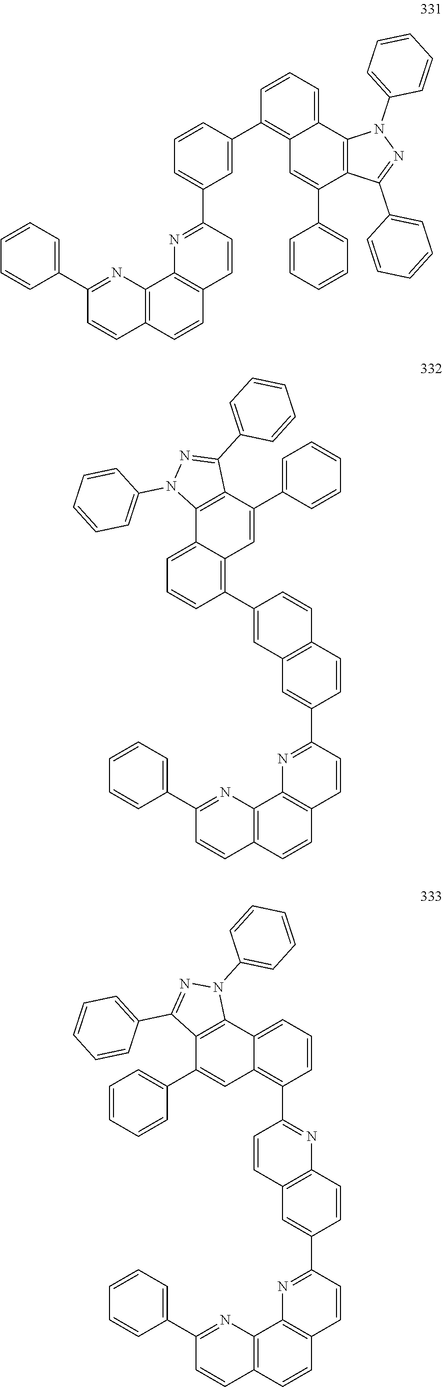 [SMILES: c1ccc(-c2ccc3ccc4ccc(-c5cccc(-c6cccc7c6cc(-c6ccccc6)c6c(-c8ccccc8)nn(-c8ccccc8)c67)c5)nc4c3n2)cc1 ; c1ccc(-c2ccc3ccc4ccc(-c5ccc6ccc(-c7cccc8c7cc(-c7ccccc7)c7c(-c9ccccc9)nn(-c9ccccc9)c78)cc6c5)nc4c3n2)cc1 ; c1ccc(-c2ccc3ccc4ccc(-c5ccc6nc(-c7cccc8c7cc(-c7ccccc7)c7c(-c9ccccc9)nn(-c9ccccc9)c78)ccc6c5)nc4c3n2)cc1]

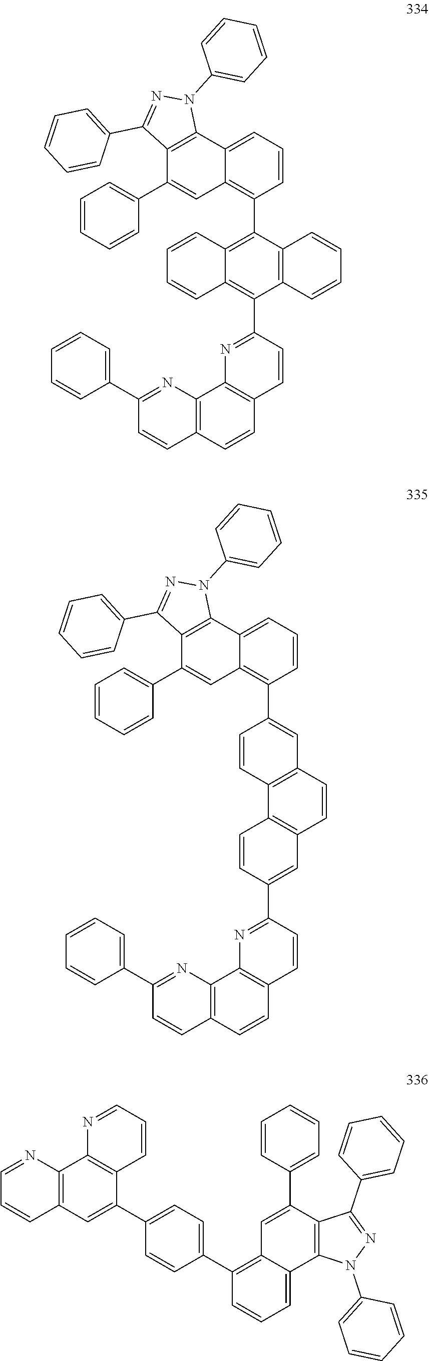 [SMILES: c1ccc(-c2ccc3ccc4ccc(-c5c6ccccc6c(-c6cccc7c6cc(-c6ccccc6)c6c(-c8ccccc8)nn(-c8ccccc8)c67)c6ccccc56)nc4c3n2)cc1 ; c1ccc(-c2ccc3ccc4ccc(-c5ccc6c(ccc7cc(-c8cccc9c8cc(-c8ccccc8)c8c(-c%10ccccc%10)nn(-c%10ccccc%10)c89)ccc76)c5)nc4c3n2)cc1 ; c1ccc(-c2cc3c(-c4ccc(-c5cc6cccnc6c6ncccc56)cc4)cccc3c3c2c(-c2ccccc2)nn3-c2ccccc2)cc1]

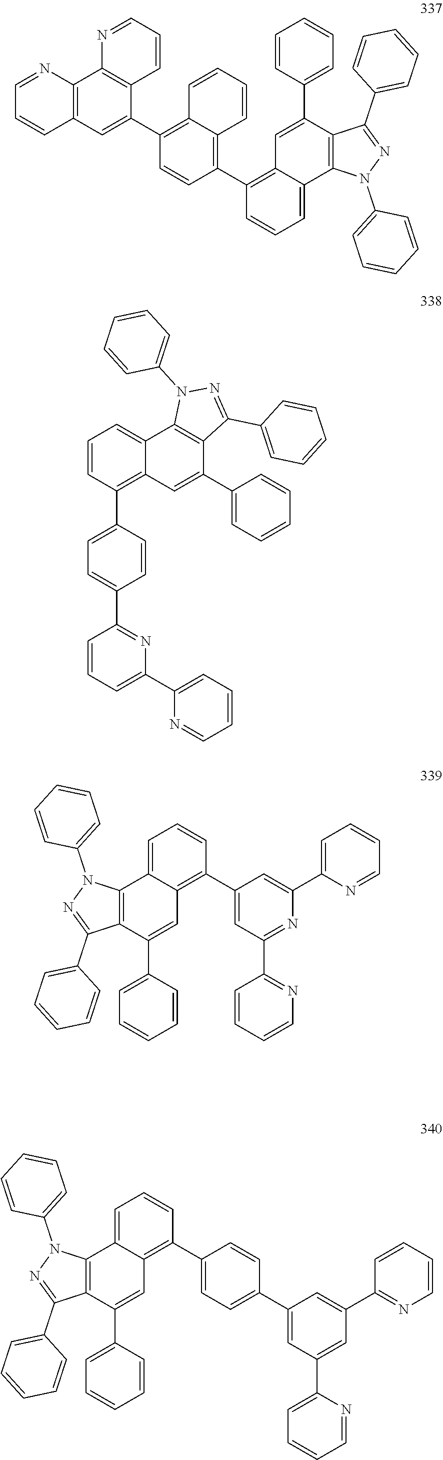 [SMILES: c1ccc(-c2cc3c(-c4ccc(-c5cc6cccnc6c6ncccc56)c5ccccc45)cccc3c3c2c(-c2ccccc2)nn3-c2ccccc2)cc1 ; c1ccc(-c2cc3c(-c4ccc(-c5cccc(-c6ccccn6)n5)cc4)cccc3c3c2c(-c2ccccc2)nn3-c2ccccc2)cc1 ; c1ccc(-c2cc3c(-c4cc(-c5ccccn5)nc(-c5ccccn5)c4)cccc3c3c2c(-c2ccccc2)nn3-c2ccccc2)cc1 ; c1ccc(-c2cc3c(-c4ccc(-c5cc(-c6ccccn6)cc(-c6ccccn6)c5)cc4)cccc3c3c2c(-c2ccccc2)nn3-c2ccccc2)cc1]

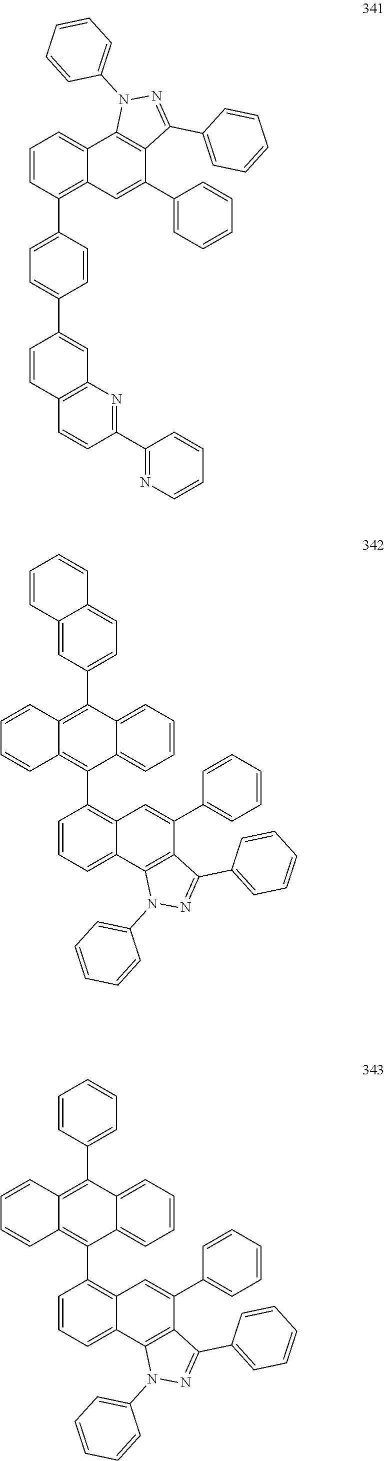 [SMILES: c1ccc(-c2cc3c(-c4ccc(-c5ccc6ccc(-c7ccccn7)nc6c5)cc4)cccc3c3c2c(-c2ccccc2)nn3-c2ccccc2)cc1 ; c1ccc(-c2cc3c(-c4c5ccccc5c(-c5ccc6ccccc6c5)c5ccccc45)cccc3c3c2c(-c2ccccc2)nn3-c2ccccc2)cc1 ; c1ccc(-c2c3ccccc3c(-c3cccc4c3cc(-c3ccccc3)c3c(-c5ccccc5)nn(-c5ccccc5)c34)c3ccccc23)cc1]

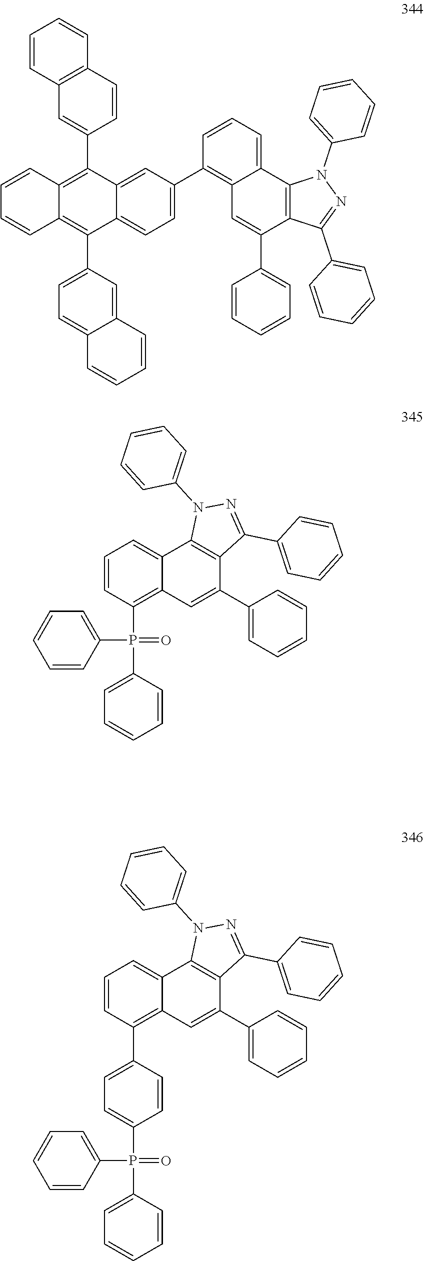 [SMILES: c1ccc(-c2cc3c(-c4ccc5c(-c6ccc7ccccc7c6)c6ccccc6c(-c6ccc7ccccc7c6)c5c4)cccc3c3c2c(-c2ccccc2)nn3-c2ccccc2)cc1 ; O=P(c1ccccc1)(c1ccccc1)c1cccc2c1cc(-c1ccccc1)c1c(-c3ccccc3)nn(-c3ccccc3)c12 ; O=P(c1ccccc1)(c1ccccc1)c1ccc(-c2cccc3c2cc(-c2ccccc2)c2c(-c4ccccc4)nn(-c4ccccc4)c23)cc1]

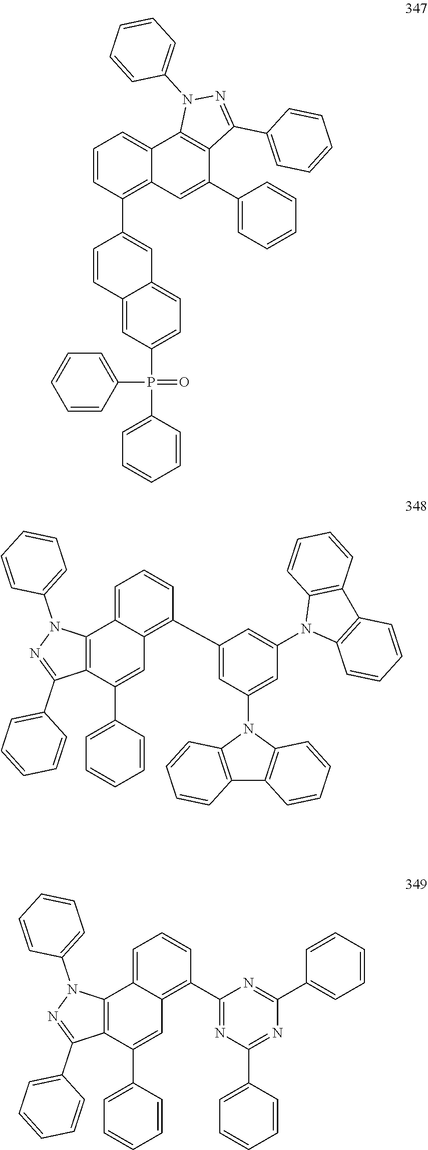 [SMILES: O=P(c1ccccc1)(c1ccccc1)c1ccc2cc(-c3cccc4c3cc(-c3ccccc3)c3c(-c5ccccc5)nn(-c5ccccc5)c34)ccc2c1 ; c1ccc(-c2cc3c(-c4cc(-n5c6ccccc6c6ccccc65)cc(-n5c6ccccc6c6ccccc65)c4)cccc3c3c2c(-c2ccccc2)nn3-c2ccccc2)cc1 ; c1ccc(-c2nc(-c3ccccc3)nc(-c3cccc4c3cc(-c3ccccc3)c3c(-c5ccccc5)nn(-c5ccccc5)c34)n2)cc1]

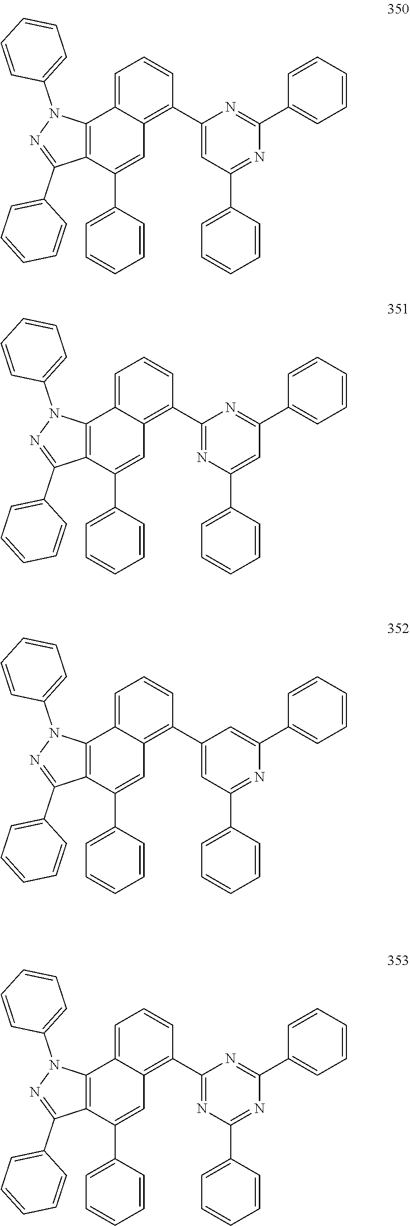 [SMILES: c1ccc(-c2cc(-c3cccc4c3cc(-c3ccccc3)c3c(-c5ccccc5)nn(-c5ccccc5)c34)nc(-c3ccccc3)n2)cc1 ; c1ccc(-c2cc(-c3ccccc3)nc(-c3cccc4c3cc(-c3ccccc3)c3c(-c5ccccc5)nn(-c5ccccc5)c34)n2)cc1 ; c1ccc(-c2cc(-c3cccc4c3cc(-c3ccccc3)c3c(-c5ccccc5)nn(-c5ccccc5)c34)cc(-c3ccccc3)n2)cc1 ; c1ccc(-c2nc(-c3ccccc3)nc(-c3cccc4c3cc(-c3ccccc3)c3c(-c5ccccc5)nn(-c5ccccc5)c34)n2)cc1]

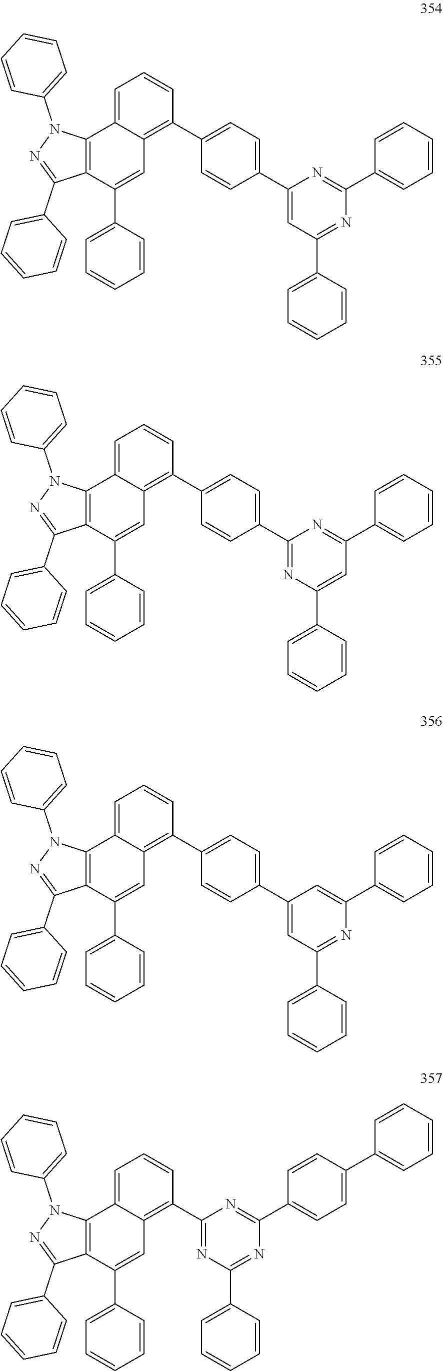 [SMILES: c1ccc(-c2cc(-c3ccc(-c4cccc5c4cc(-c4ccccc4)c4c(-c6ccccc6)nn(-c6ccccc6)c45)cc3)nc(-c3ccccc3)n2)cc1 ; c1ccc(-c2cc(-c3ccccc3)nc(-c3ccc(-c4cccc5c4cc(-c4ccccc4)c4c(-c6ccccc6)nn(-c6ccccc6)c45)cc3)n2)cc1 ; c1ccc(-c2cc(-c3ccc(-c4cccc5c4cc(-c4ccccc4)c4c(-c6ccccc6)nn(-c6ccccc6)c45)cc3)cc(-c3ccccc3)n2)cc1 ; c1ccc(-c2ccc(-c3nc(-c4ccccc4)nc(-c4cccc5c4cc(-c4ccccc4)c4c(-c6ccccc6)nn(-c6ccccc6)c45)n3)cc2)cc1]

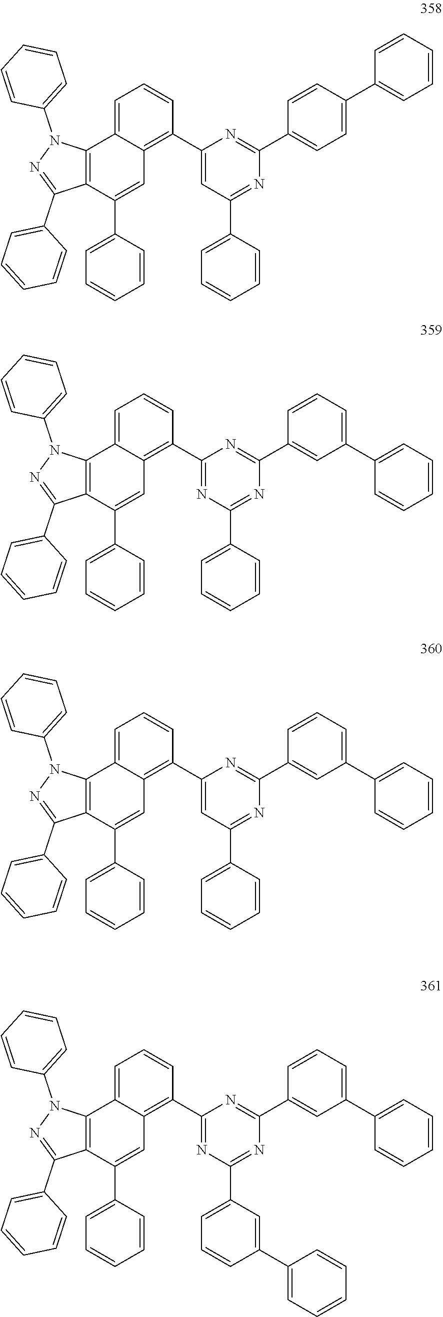 [SMILES: c1ccc(-c2ccc(-c3nc(-c4ccccc4)cc(-c4cccc5c4cc(-c4ccccc4)c4c(-c6ccccc6)nn(-c6ccccc6)c45)n3)cc2)cc1 ; c1ccc(-c2cccc(-c3nc(-c4ccccc4)nc(-c4cccc5c4cc(-c4ccccc4)c4c(-c6ccccc6)nn(-c6ccccc6)c45)n3)c2)cc1 ; c1ccc(-c2cccc(-c3nc(-c4ccccc4)cc(-c4cccc5c4cc(-c4ccccc4)c4c(-c6ccccc6)nn(-c6ccccc6)c45)n3)c2)cc1 ; c1ccc(-c2cccc(-c3nc(-c4cccc(-c5ccccc5)c4)nc(-c4cccc5c4cc(-c4ccccc4)c4c(-c6ccccc6)nn(-c6ccccc6)c45)n3)c2)cc1]

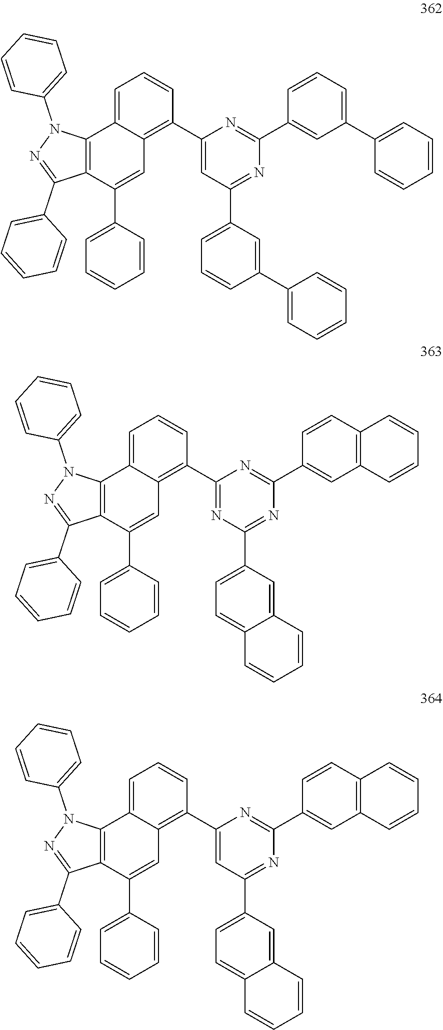 [SMILES: c1ccc(-c2cccc(-c3cc(-c4cccc5c4cc(-c4ccccc4)c4c(-c6ccccc6)nn(-c6ccccc6)c45)nc(-c4cccc(-c5ccccc5)c4)n3)c2)cc1 ; c1ccc(-c2cc3c(-c4nc(-c5ccc6ccccc6c5)nc(-c5ccc6ccccc6c5)n4)cccc3c3c2c(-c2ccccc2)nn3-c2ccccc2)cc1 ; c1ccc(-c2cc3c(-c4cc(-c5ccc6ccccc6c5)nc(-c5ccc6ccccc6c5)n4)cccc3c3c2c(-c2ccccc2)nn3-c2ccccc2)cc1]

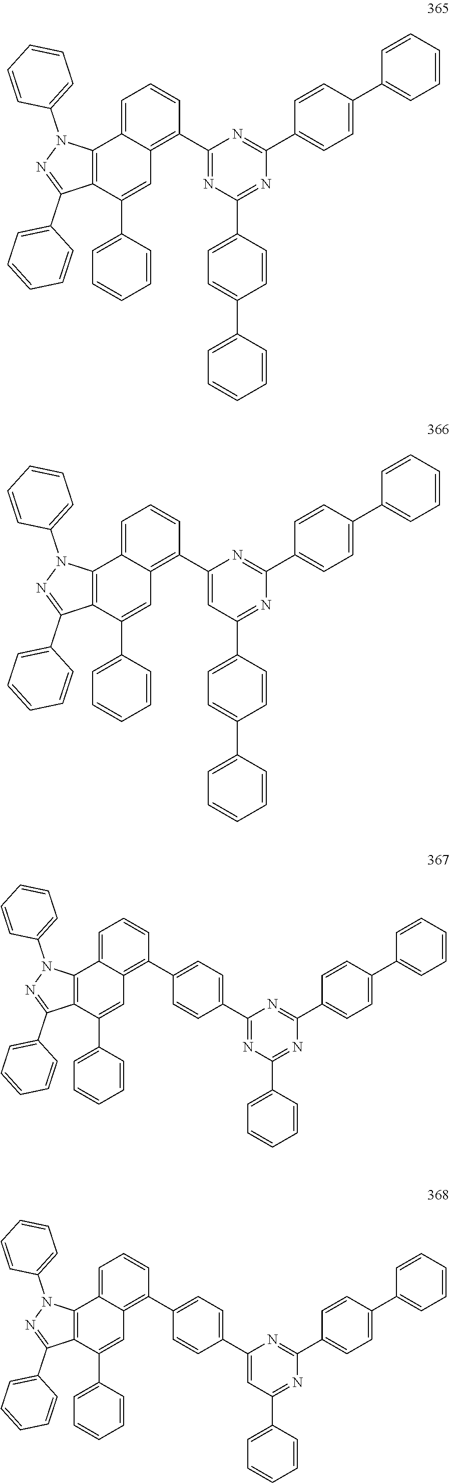 [SMILES: c1ccc(-c2ccc(-c3nc(-c4ccc(-c5ccccc5)cc4)nc(-c4cccc5c4cc(-c4ccccc4)c4c(-c6ccccc6)nn(-c6ccccc6)c45)n3)cc2)cc1 ; c1ccc(-c2ccc(-c3cc(-c4cccc5c4cc(-c4ccccc4)c4c(-c6ccccc6)nn(-c6ccccc6)c45)nc(-c4ccc(-c5ccccc5)cc4)n3)cc2)cc1 ; c1ccc(-c2ccc(-c3nc(-c4ccccc4)nc(-c4ccc(-c5cccc6c5cc(-c5ccccc5)c5c(-c7ccccc7)nn(-c7ccccc7)c56)cc4)n3)cc2)cc1 ; c1ccc(-c2ccc(-c3nc(-c4ccccc4)cc(-c4ccc(-c5cccc6c5cc(-c5ccccc5)c5c(-c7ccccc7)nn(-c7ccccc7)c56)cc4)n3)cc2)cc1]

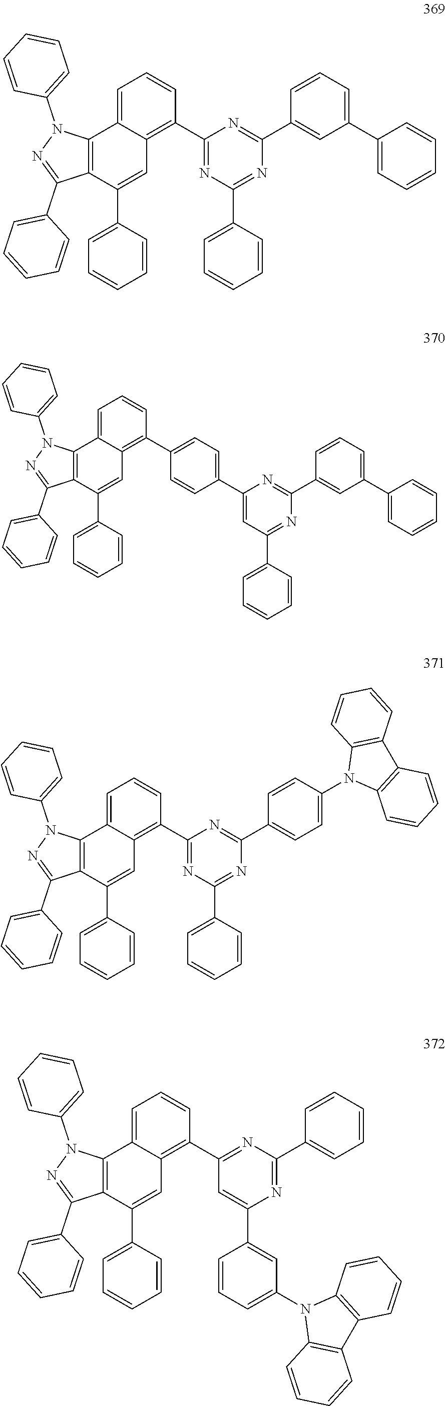 [SMILES: c1ccc(-c2cccc(-c3nc(-c4ccccc4)nc(-c4cccc5c4cc(-c4ccccc4)c4c(-c6ccccc6)nn(-c6ccccc6)c45)n3)c2)cc1 ; c1ccc(-c2cccc(-c3nc(-c4ccccc4)cc(-c4ccc(-c5cccc6c5cc(-c5ccccc5)c5c(-c7ccccc7)nn(-c7ccccc7)c56)cc4)n3)c2)cc1 ; c1ccc(-c2nc(-c3ccc(-n4c5ccccc5c5ccccc54)cc3)nc(-c3cccc4c3cc(-c3ccccc3)c3c(-c5ccccc5)nn(-c5ccccc5)c34)n2)cc1 ; c1ccc(-c2nc(-c3cccc(-n4c5ccccc5c5ccccc54)c3)cc(-c3cccc4c3cc(-c3ccccc3)c3c(-c5ccccc5)nn(-c5ccccc5)c34)n2)cc1]

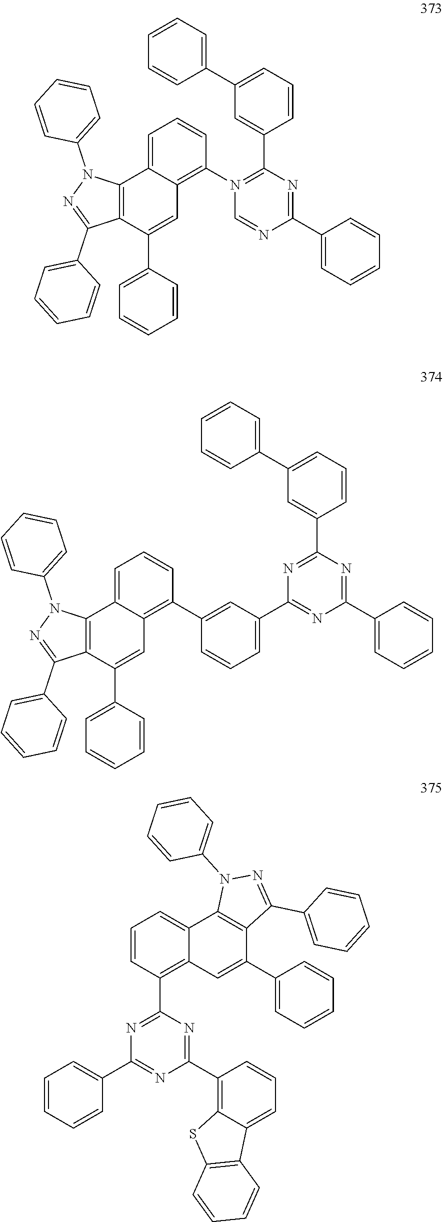

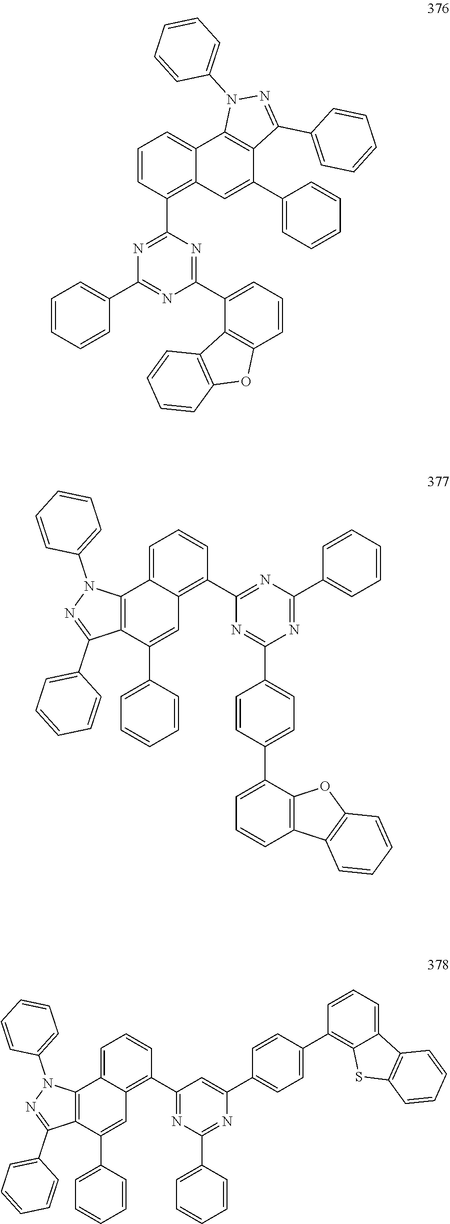 [SMILES: c1ccc(-c2nc(-c3cccc4c3cc(-c3ccccc3)c3c(-c5ccccc5)nn(-c5ccccc5)c34)nc(-c3cccc4oc5ccccc5c34)n2)cc1 ; c1ccc(-c2nc(-c3ccc(-c4cccc5c4oc4ccccc45)cc3)nc(-c3cccc4c3cc(-c3ccccc3)c3c(-c5ccccc5)nn(-c5ccccc5)c34)n2)cc1 ; c1ccc(-c2nc(-c3ccc(-c4cccc5c4sc4ccccc45)cc3)cc(-c3cccc4c3cc(-c3ccccc3)c3c(-c5ccccc5)nn(-c5ccccc5)c34)n2)cc1]

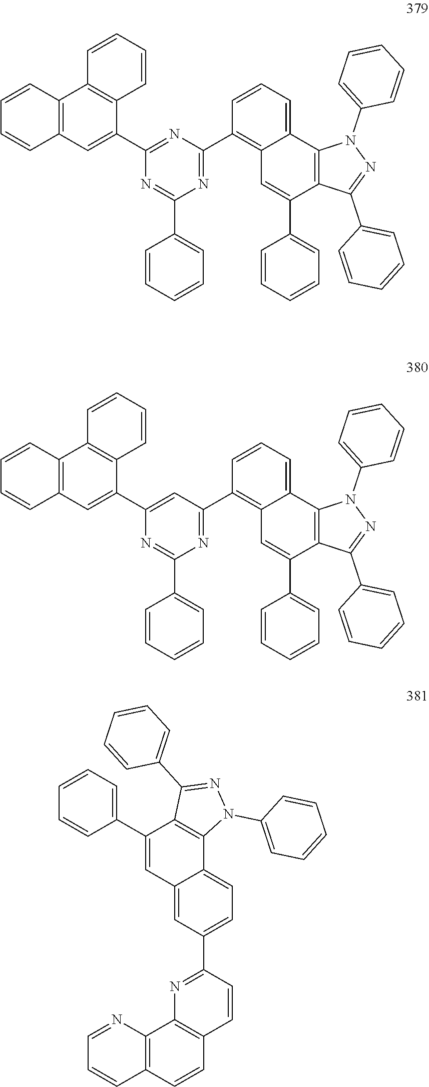 [SMILES: c1ccc(-c2nc(-c3cc4ccccc4c4ccccc34)nc(-c3cccc4c3cc(-c3ccccc3)c3c(-c5ccccc5)nn(-c5ccccc5)c34)n2)cc1 ; c1ccc(-c2nc(-c3cc4ccccc4c4ccccc34)cc(-c3cccc4c3cc(-c3ccccc3)c3c(-c5ccccc5)nn(-c5ccccc5)c34)n2)cc1 ; c1ccc(-c2cc3cc(-c4ccc5ccc6cccnc6c5n4)ccc3c3c2c(-c2ccccc2)nn3-c2ccccc2)cc1]

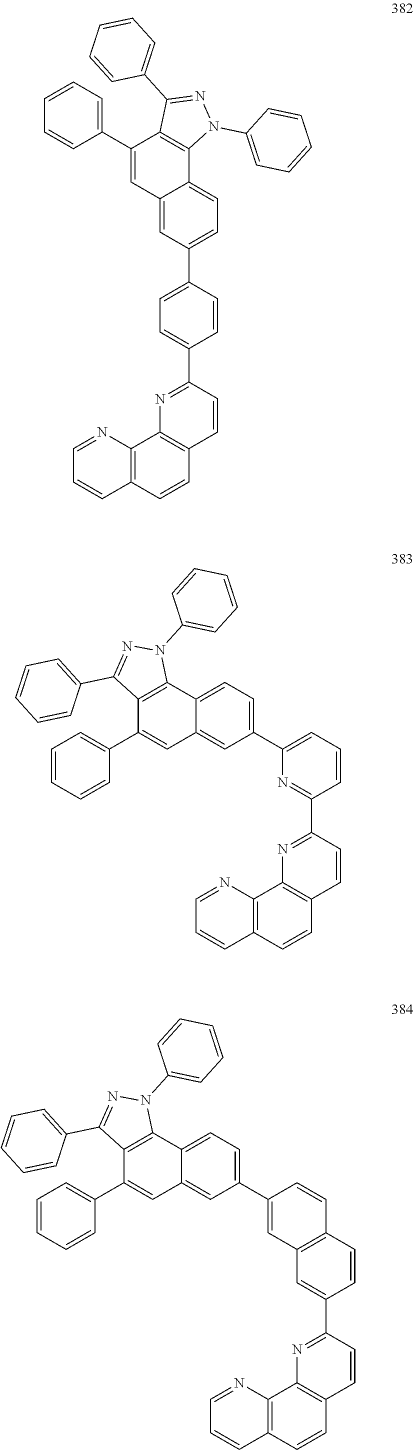 [SMILES: c1ccc(-c2cc3cc(-c4ccc(-c5ccc6ccc7cccnc7c6n5)cc4)ccc3c3c2c(-c2ccccc2)nn3-c2ccccc2)cc1 ; c1ccc(-c2cc3cc(-c4cccc(-c5ccc6ccc7cccnc7c6n5)n4)ccc3c3c2c(-c2ccccc2)nn3-c2ccccc2)cc1 ; c1ccc(-c2cc3cc(-c4ccc5ccc(-c6ccc7ccc8cccnc8c7n6)cc5c4)ccc3c3c2c(-c2ccccc2)nn3-c2ccccc2)cc1]

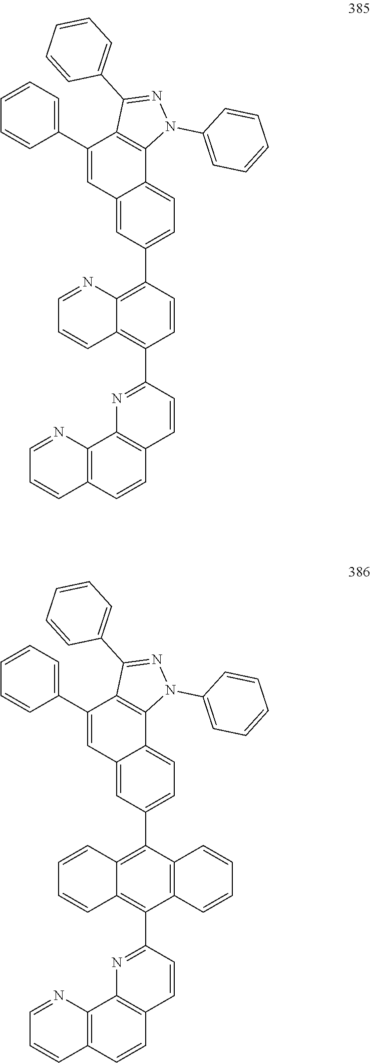 [SMILES: c1ccc(-c2cc3cc(-c4ccc(-c5ccc6ccc7cccnc7c6n5)c5cccnc45)ccc3c3c2c(-c2ccccc2)nn3-c2ccccc2)cc1 ; c1ccc(-c2cc3cc(-c4c5ccccc5c(-c5ccc6ccc7cccnc7c6n5)c5ccccc45)ccc3c3c2c(-c2ccccc2)nn3-c2ccccc2)cc1]

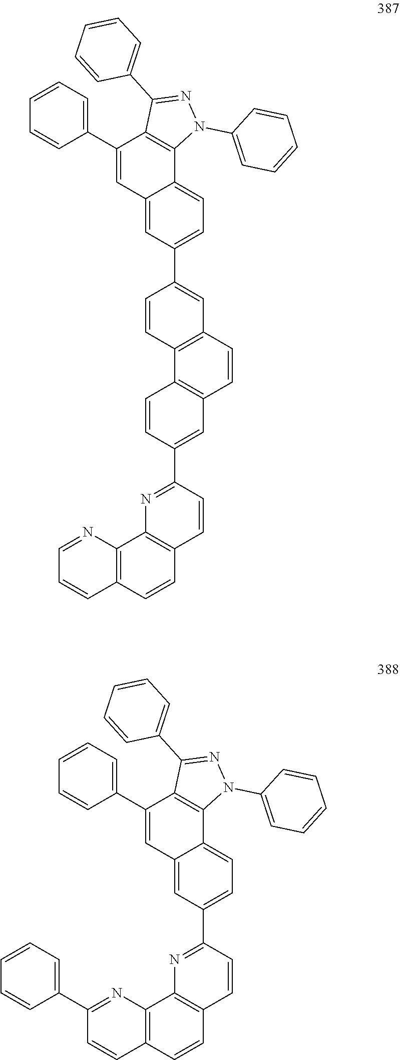 [SMILES: c1ccc(-c2cc3cc(-c4ccc5c(ccc6cc(-c7ccc8ccc9cccnc9c8n7)ccc65)c4)ccc3c3c2c(-c2ccccc2)nn3-c2ccccc2)cc1 ; c1ccc(-c2ccc3ccc4ccc(-c5ccc6c(c5)cc(-c5ccccc5)c5c(-c7ccccc7)nn(-c7ccccc7)c56)nc4c3n2)cc1]

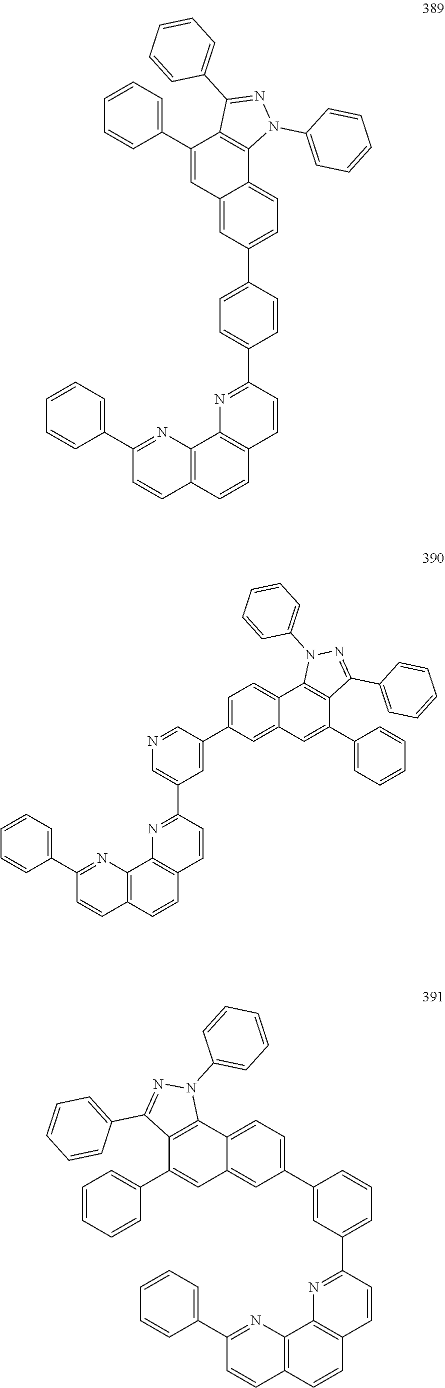 [SMILES: c1ccc(-c2ccc3ccc4ccc(-c5ccc(-c6ccc7c(c6)cc(-c6ccccc6)c6c(-c8ccccc8)nn(-c8ccccc8)c67)cc5)nc4c3n2)cc1 ; c1ccc(-c2ccc3ccc4ccc(-c5cncc(-c6ccc7c(c6)cc(-c6ccccc6)c6c(-c8ccccc8)nn(-c8ccccc8)c67)c5)nc4c3n2)cc1 ; c1ccc(-c2ccc3ccc4ccc(-c5cccc(-c6ccc7c(c6)cc(-c6ccccc6)c6c(-c8ccccc8)nn(-c8ccccc8)c67)c5)nc4c3n2)cc1]

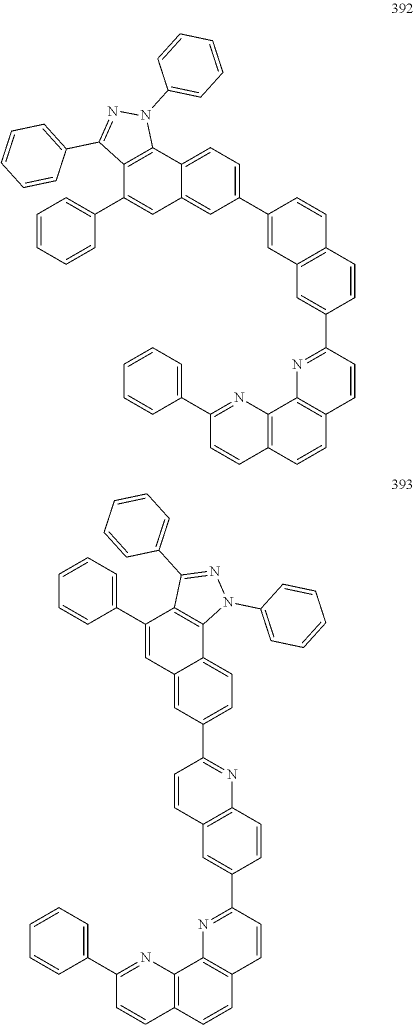 [SMILES: c1ccc(-c2ccc3ccc4ccc(-c5ccc6ccc(-c7ccc8c(c7)cc(-c7ccccc7)c7c(-c9ccccc9)nn(-c9ccccc9)c78)cc6c5)nc4c3n2)cc1 ; c1ccc(-c2ccc3ccc4ccc(-c5ccc6nc(-c7ccc8c(c7)cc(-c7ccccc7)c7c(-c9ccccc9)nn(-c9ccccc9)c78)ccc6c5)nc4c3n2)cc1]

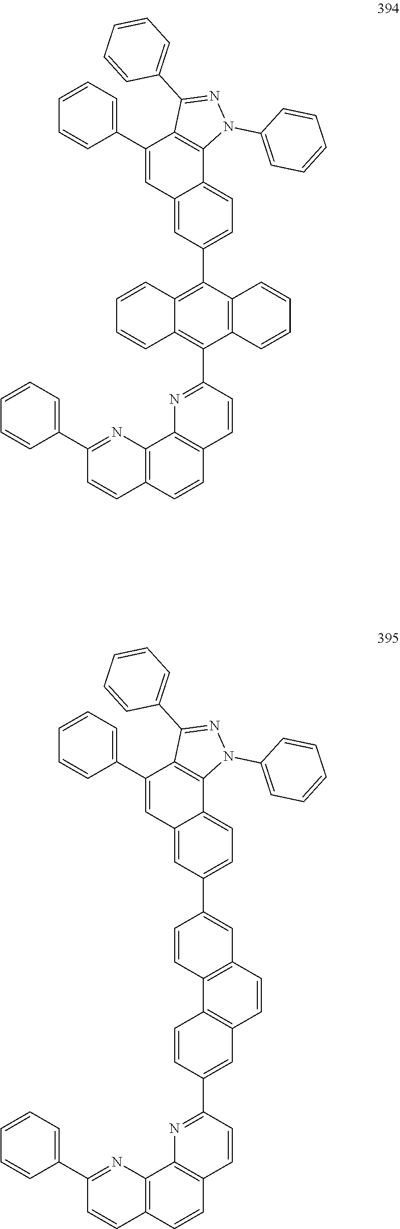 [SMILES: c1ccc(-c2ccc3ccc4ccc(-c5c6ccccc6c(-c6ccc7c(c6)cc(-c6ccccc6)c6c(-c8ccccc8)nn(-c8ccccc8)c67)c6ccccc56)nc4c3n2)cc1 ; c1ccc(-c2ccc3ccc4ccc(-c5ccc6c(ccc7cc(-c8ccc9c(c8)cc(-c8ccccc8)c8c(-c%10ccccc%10)nn(-c%10ccccc%10)c89)ccc76)c5)nc4c3n2)cc1]

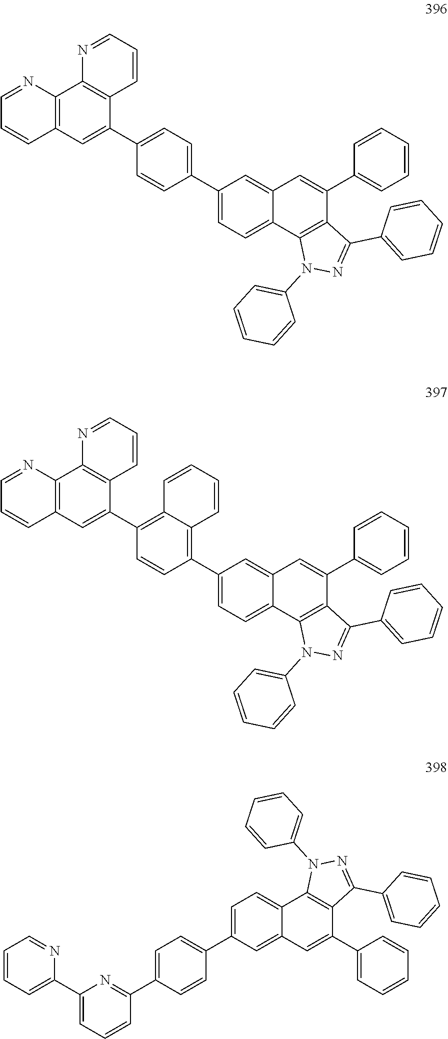 [SMILES: c1ccc(-c2cc3cc(-c4ccc(-c5cc6cccnc6c6ncccc56)cc4)ccc3c3c2c(-c2ccccc2)nn3-c2ccccc2)cc1 ; c1ccc(-c2cc3cc(-c4ccc(-c5cc6cccnc6c6ncccc56)c5ccccc45)ccc3c3c2c(-c2ccccc2)nn3-c2ccccc2)cc1 ; c1ccc(-c2cc3cc(-c4ccc(-c5cccc(-c6ccccn6)n5)cc4)ccc3c3c2c(-c2ccccc2)nn3-c2ccccc2)cc1]

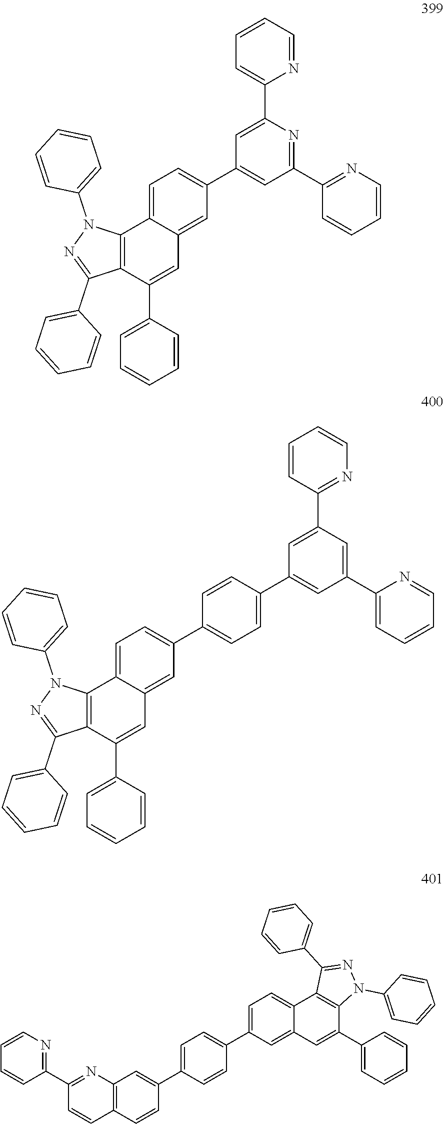 [SMILES: c1ccc(-c2cc3cc(-c4cc(-c5ccccn5)nc(-c5ccccn5)c4)ccc3c3c2c(-c2ccccc2)nn3-c2ccccc2)cc1 ; c1ccc(-c2cc3cc(-c4ccc(-c5cc(-c6ccccn6)cc(-c6ccccn6)c5)cc4)ccc3c3c2c(-c2ccccc2)nn3-c2ccccc2)cc1 ; c1ccc(-c2nn(-c3ccccc3)c3c(-c4ccccc4)cc4cc(-c5ccc(-c6ccc7ccc(-c8ccccn8)nc7c6)cc5)ccc4c23)cc1]

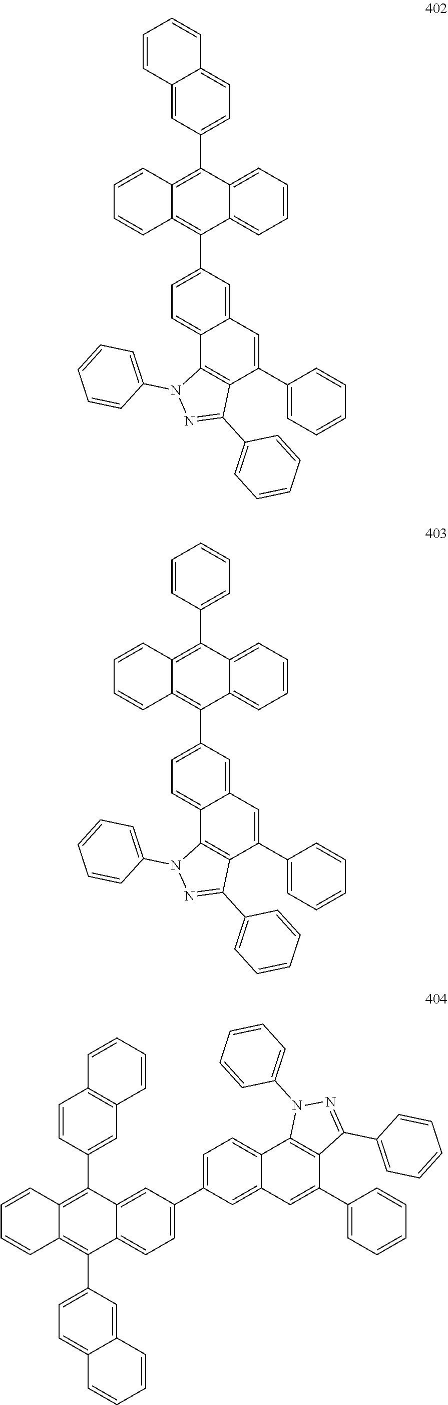 [SMILES: c1ccc(-c2cc3cc(-c4c5ccccc5c(-c5ccc6ccccc6c5)c5ccccc45)ccc3c3c2c(-c2ccccc2)nn3-c2ccccc2)cc1 ; c1ccc(-c2c3ccccc3c(-c3ccc4c(c3)cc(-c3ccccc3)c3c(-c5ccccc5)nn(-c5ccccc5)c34)c3ccccc23)cc1 ; c1ccc(-c2cc3cc(-c4ccc5c(-c6ccc7ccccc7c6)c6ccccc6c(-c6ccc7ccccc7c6)c5c4)ccc3c3c2c(-c2ccccc2)nn3-c2ccccc2)cc1]

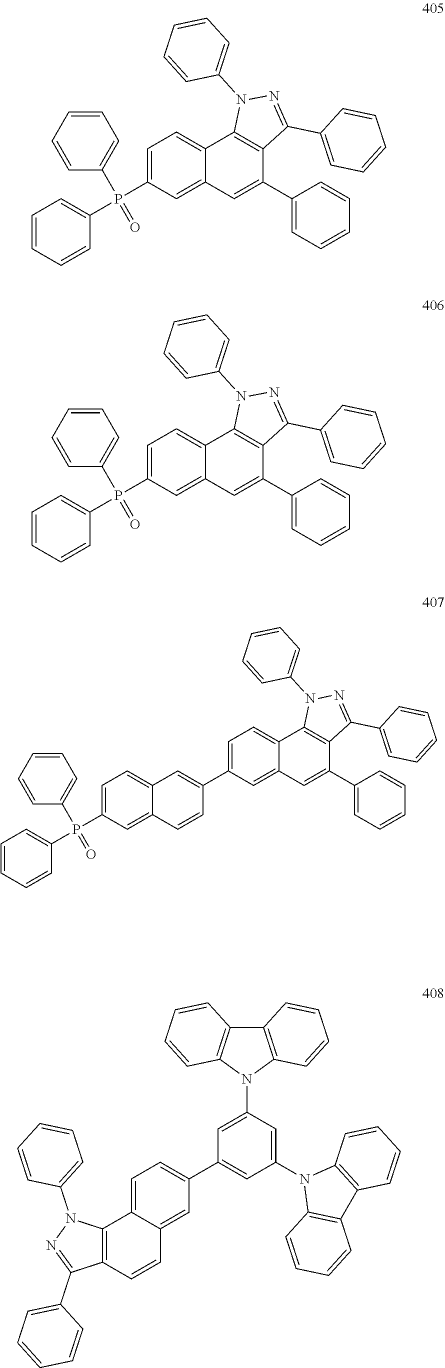 [SMILES: O=P(c1ccccc1)(c1ccccc1)c1ccc2c(c1)cc(-c1ccccc1)c1c(-c3ccccc3)nn(-c3ccccc3)c12 ; O=P(c1ccccc1)(c1ccccc1)c1ccc2c(c1)cc(-c1ccccc1)c1c(-c3ccccc3)nn(-c3ccccc3)c12 ; O=P(c1ccccc1)(c1ccccc1)c1ccc2cc(-c3ccc4c(c3)cc(-c3ccccc3)c3c(-c5ccccc5)nn(-c5ccccc5)c34)ccc2c1 ; c1ccc(-c2nn(-c3ccccc3)c3c2ccc2cc(-c4cc(-n5c6ccccc6c6ccccc65)cc(-n5c6ccccc6c6ccccc65)c4)ccc23)cc1]

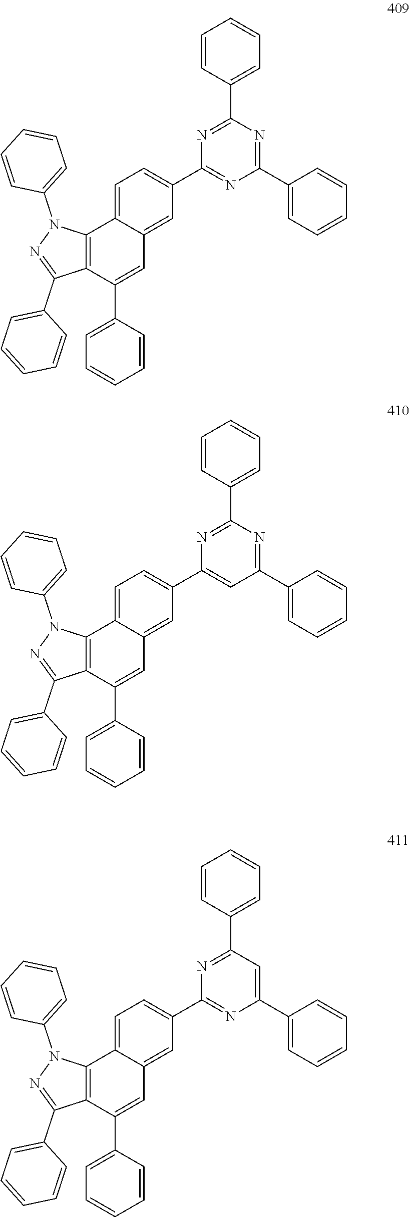 [SMILES: c1ccc(-c2nc(-c3ccccc3)nc(-c3ccc4c(c3)cc(-c3ccccc3)c3c(-c5ccccc5)nn(-c5ccccc5)c34)n2)cc1 ; c1ccc(-c2cc(-c3ccc4c(c3)cc(-c3ccccc3)c3c(-c5ccccc5)nn(-c5ccccc5)c34)nc(-c3ccccc3)n2)cc1 ; c1ccc(-c2cc(-c3ccccc3)nc(-c3ccc4c(c3)cc(-c3ccccc3)c3c(-c5ccccc5)nn(-c5ccccc5)c34)n2)cc1]

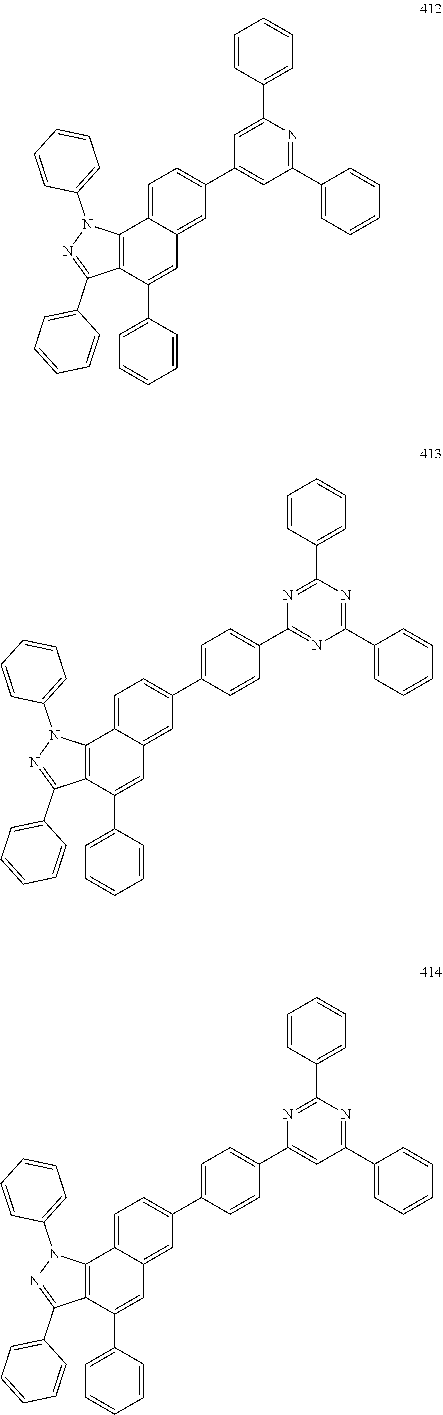 [SMILES: c1ccc(-c2cc(-c3ccc4c(c3)cc(-c3ccccc3)c3c(-c5ccccc5)nn(-c5ccccc5)c34)cc(-c3ccccc3)n2)cc1 ; c1ccc(-c2nc(-c3ccccc3)nc(-c3ccc(-c4ccc5c(c4)cc(-c4ccccc4)c4c(-c6ccccc6)nn(-c6ccccc6)c45)cc3)n2)cc1 ; c1ccc(-c2cc(-c3ccc(-c4ccc5c(c4)cc(-c4ccccc4)c4c(-c6ccccc6)nn(-c6ccccc6)c45)cc3)nc(-c3ccccc3)n2)cc1]

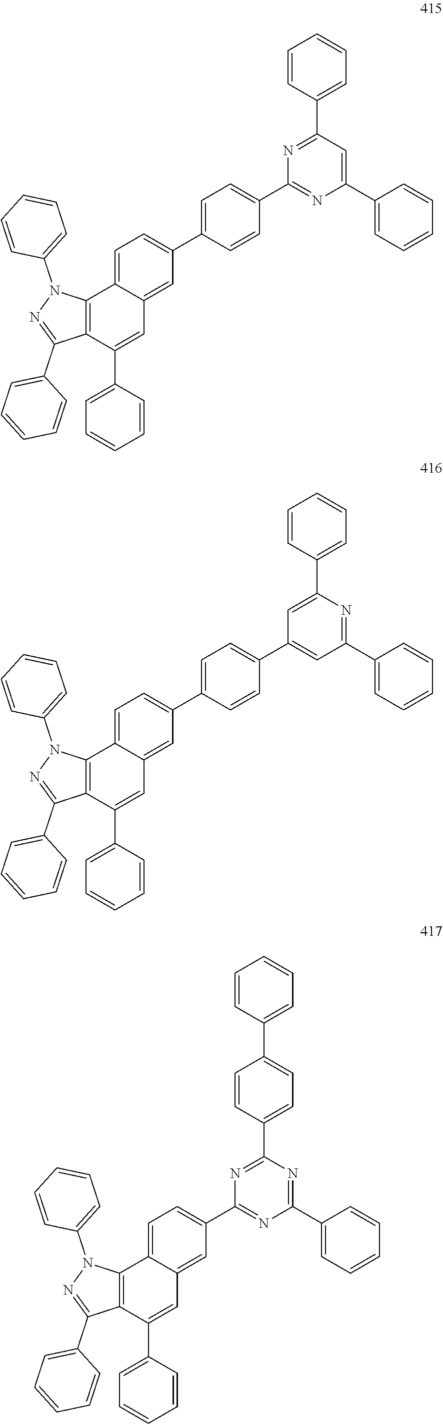 [SMILES: c1ccc(-c2cc(-c3ccccc3)nc(-c3ccc(-c4ccc5c(c4)cc(-c4ccccc4)c4c(-c6ccccc6)nn(-c6ccccc6)c45)cc3)n2)cc1 ; c1ccc(-c2cc(-c3ccc(-c4ccc5c(c4)cc(-c4ccccc4)c4c(-c6ccccc6)nn(-c6ccccc6)c45)cc3)cc(-c3ccccc3)n2)cc1 ; c1ccc(-c2ccc(-c3nc(-c4ccccc4)nc(-c4ccc5c(c4)cc(-c4ccccc4)c4c(-c6ccccc6)nn(-c6ccccc6)c45)n3)cc2)cc1]

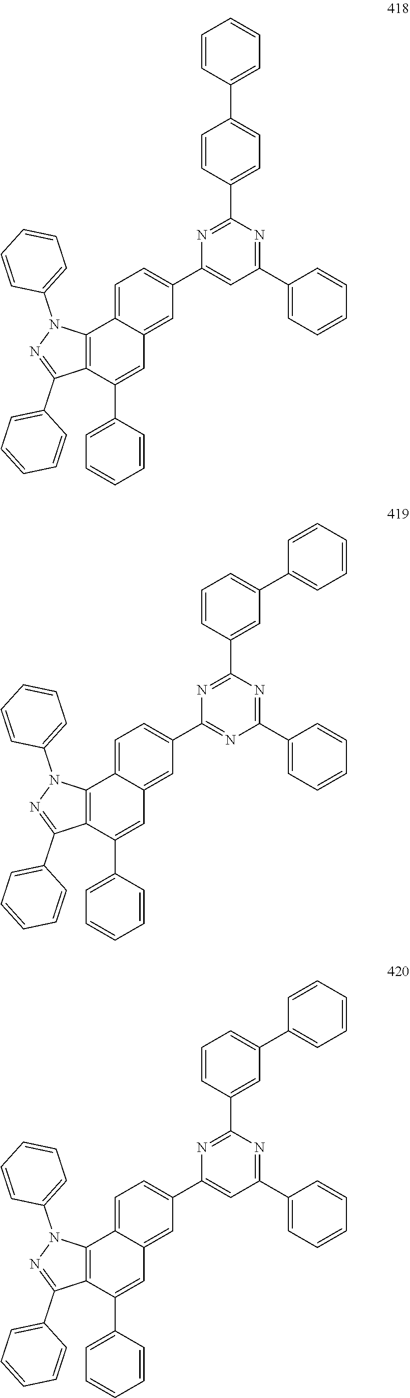 [SMILES: c1ccc(-c2ccc(-c3nc(-c4ccccc4)cc(-c4ccc5c(c4)cc(-c4ccccc4)c4c(-c6ccccc6)nn(-c6ccccc6)c45)n3)cc2)cc1 ; c1ccc(-c2cccc(-c3nc(-c4ccccc4)nc(-c4ccc5c(c4)cc(-c4ccccc4)c4c(-c6ccccc6)nn(-c6ccccc6)c45)n3)c2)cc1 ; c1ccc(-c2cccc(-c3nc(-c4ccccc4)cc(-c4ccc5c(c4)cc(-c4ccccc4)c4c(-c6ccccc6)nn(-c6ccccc6)c45)n3)c2)cc1]

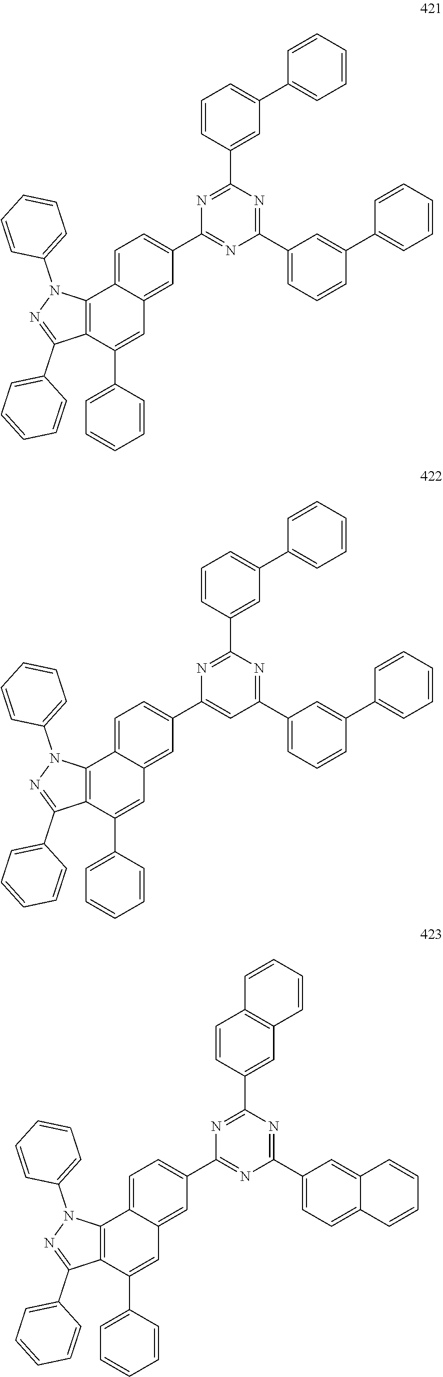 [SMILES: c1ccc(-c2cccc(-c3nc(-c4cccc(-c5ccccc5)c4)nc(-c4ccc5c(c4)cc(-c4ccccc4)c4c(-c6ccccc6)nn(-c6ccccc6)c45)n3)c2)cc1 ; c1ccc(-c2cccc(-c3cc(-c4ccc5c(c4)cc(-c4ccccc4)c4c(-c6ccccc6)nn(-c6ccccc6)c45)nc(-c4cccc(-c5ccccc5)c4)n3)c2)cc1 ; c1ccc(-c2cc3cc(-c4nc(-c5ccc6ccccc6c5)nc(-c5ccc6ccccc6c5)n4)ccc3c3c2c(-c2ccccc2)nn3-c2ccccc2)cc1]

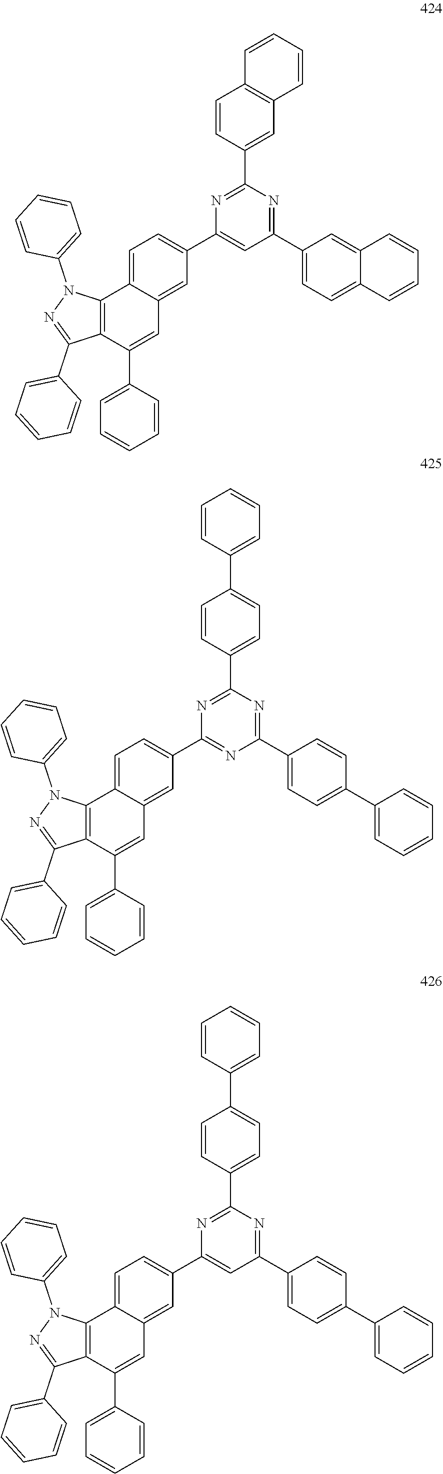 [SMILES: c1ccc(-c2cc3cc(-c4cc(-c5ccc6ccccc6c5)nc(-c5ccc6ccccc6c5)n4)ccc3c3c2c(-c2ccccc2)nn3-c2ccccc2)cc1 ; c1ccc(-c2ccc(-c3nc(-c4ccc(-c5ccccc5)cc4)nc(-c4ccc5c(c4)cc(-c4ccccc4)c4c(-c6ccccc6)nn(-c6ccccc6)c45)n3)cc2)cc1 ; c1ccc(-c2ccc(-c3cc(-c4ccc5c(c4)cc(-c4ccccc4)c4c(-c6ccccc6)nn(-c6ccccc6)c45)nc(-c4ccc(-c5ccccc5)cc4)n3)cc2)cc1]

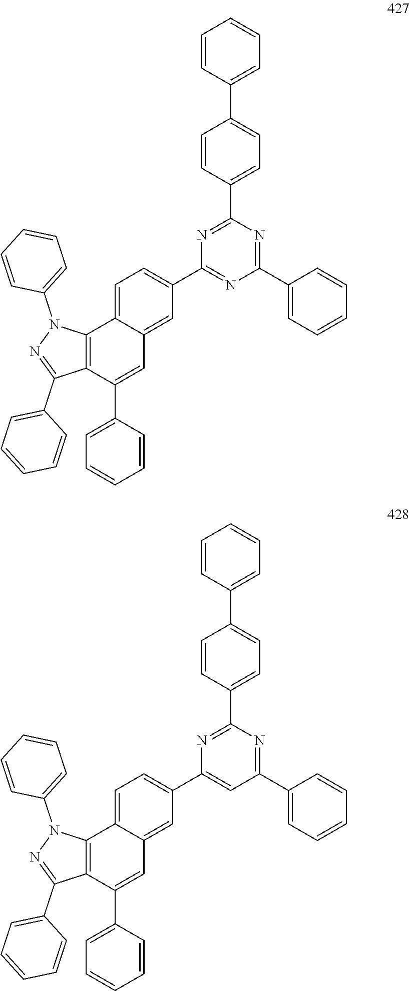 [SMILES: c1ccc(-c2ccc(-c3nc(-c4ccccc4)nc(-c4ccc5c(c4)cc(-c4ccccc4)c4c(-c6ccccc6)nn(-c6ccccc6)c45)n3)cc2)cc1 ; c1ccc(-c2ccc(-c3nc(-c4ccccc4)cc(-c4ccc5c(c4)cc(-c4ccccc4)c4c(-c6ccccc6)nn(-c6ccccc6)c45)n3)cc2)cc1]

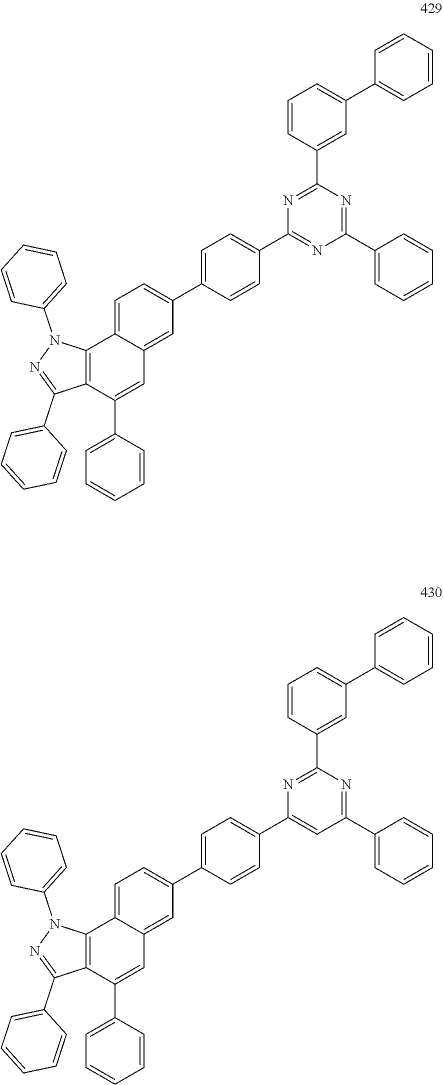 [SMILES: c1ccc(-c2cccc(-c3nc(-c4ccccc4)nc(-c4ccc(-c5ccc6c(c5)cc(-c5ccccc5)c5c(-c7ccccc7)nn(-c7ccccc7)c56)cc4)n3)c2)cc1 ; c1ccc(-c2cccc(-c3nc(-c4ccccc4)cc(-c4ccc(-c5ccc6c(c5)cc(-c5ccccc5)c5c(-c7ccccc7)nn(-c7ccccc7)c56)cc4)n3)c2)cc1]

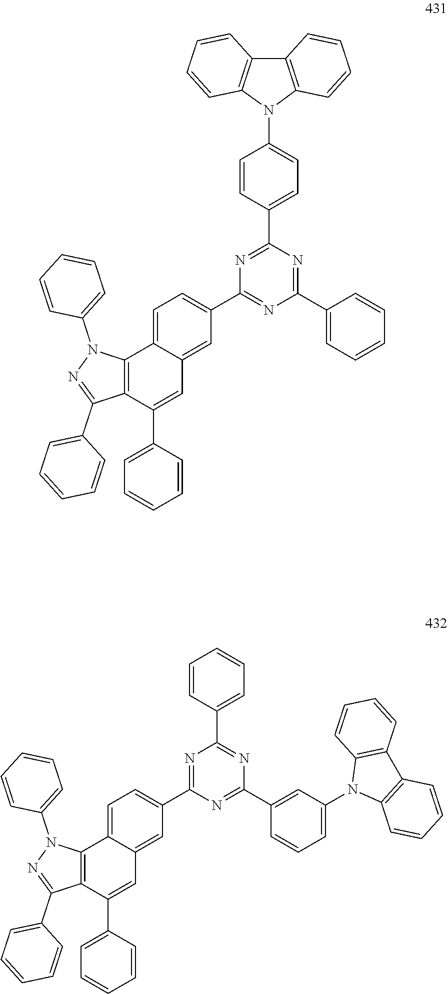 [SMILES: c1ccc(-c2nc(-c3ccc(-n4c5ccccc5c5ccccc54)cc3)nc(-c3ccc4c(c3)cc(-c3ccccc3)c3c(-c5ccccc5)nn(-c5ccccc5)c34)n2)cc1 ; c1ccc(-c2nc(-c3cccc(-n4c5ccccc5c5ccccc54)c3)nc(-c3ccc4c(c3)cc(-c3ccccc3)c3c(-c5ccccc5)nn(-c5ccccc5)c34)n2)cc1]

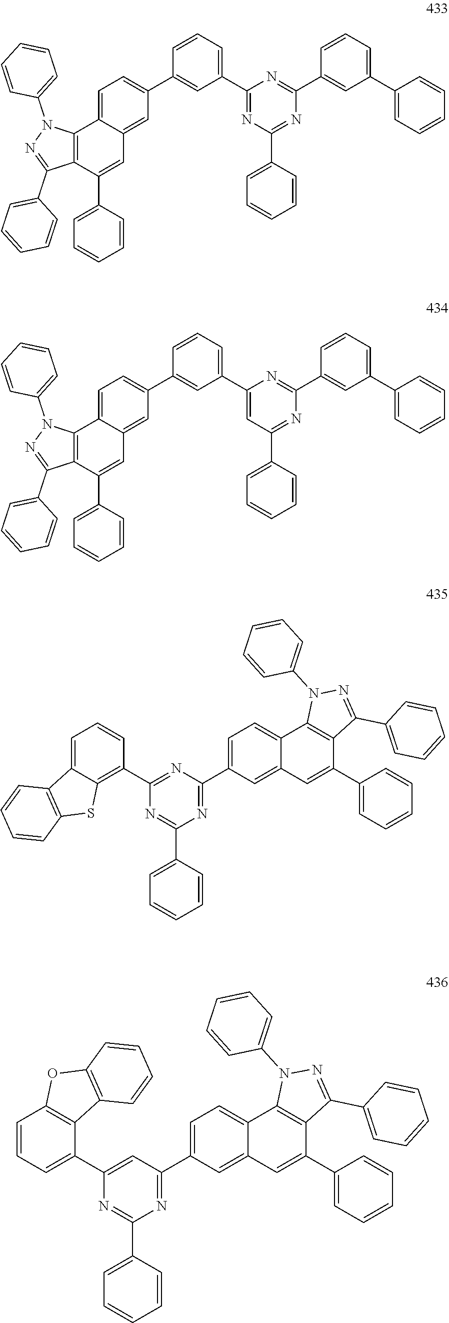 [SMILES: c1ccc(-c2cccc(-c3nc(-c4ccccc4)nc(-c4cccc(-c5ccc6c(c5)cc(-c5ccccc5)c5c(-c7ccccc7)nn(-c7ccccc7)c56)c4)n3)c2)cc1 ; c1ccc(-c2cccc(-c3nc(-c4ccccc4)cc(-c4cccc(-c5ccc6c(c5)cc(-c5ccccc5)c5c(-c7ccccc7)nn(-c7ccccc7)c56)c4)n3)c2)cc1 ; c1ccc(-c2nc(-c3ccc4c(c3)cc(-c3ccccc3)c3c(-c5ccccc5)nn(-c5ccccc5)c34)nc(-c3cccc4c3sc3ccccc34)n2)cc1 ; c1ccc(-c2nc(-c3ccc4c(c3)cc(-c3ccccc3)c3c(-c5ccccc5)nn(-c5ccccc5)c34)cc(-c3cccc4oc5ccccc5c34)n2)cc1]

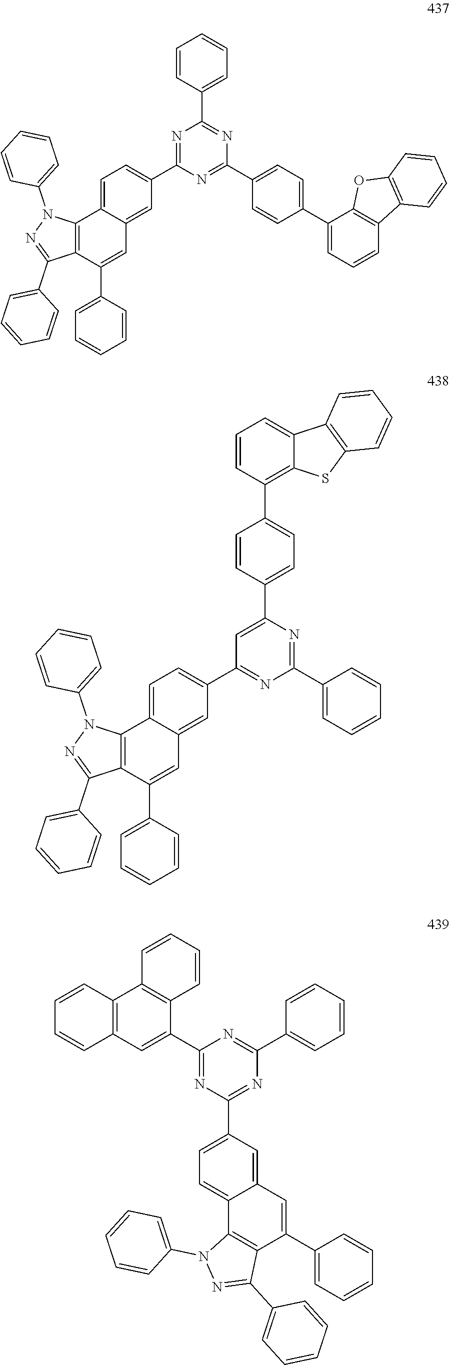 [SMILES: c1ccc(-c2nc(-c3ccc(-c4cccc5c4oc4ccccc45)cc3)nc(-c3ccc4c(c3)cc(-c3ccccc3)c3c(-c5ccccc5)nn(-c5ccccc5)c34)n2)cc1 ; c1ccc(-c2nc(-c3ccc(-c4cccc5c4sc4ccccc45)cc3)cc(-c3ccc4c(c3)cc(-c3ccccc3)c3c(-c5ccccc5)nn(-c5ccccc5)c34)n2)cc1 ; c1ccc(-c2nc(-c3ccc4c(c3)cc(-c3ccccc3)c3c(-c5ccccc5)nn(-c5ccccc5)c34)nc(-c3cc4ccccc4c4ccccc34)n2)cc1]

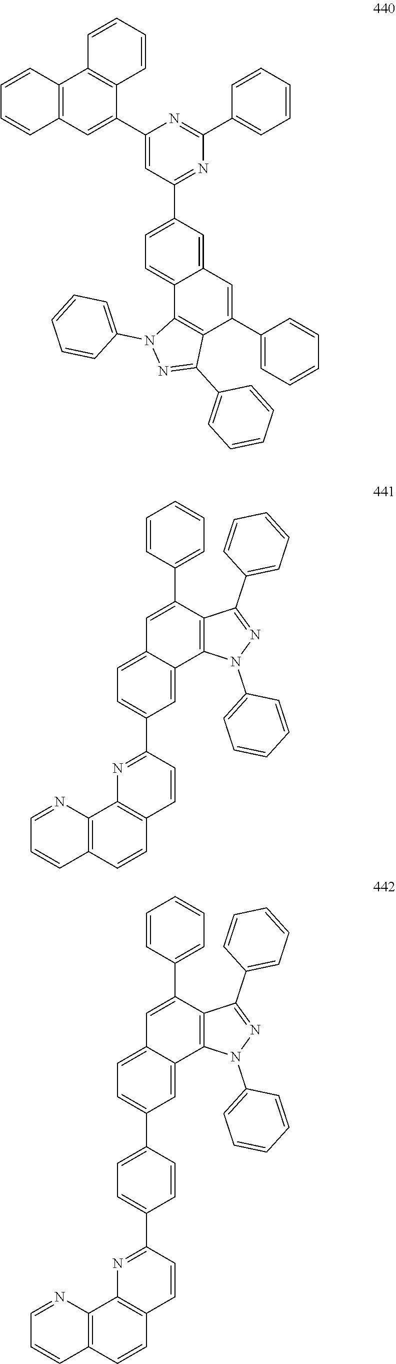 [SMILES: c1ccc(-c2nc(-c3ccc4c(c3)cc(-c3ccccc3)c3c(-c5ccccc5)nn(-c5ccccc5)c34)cc(-c3cc4ccccc4c4ccccc34)n2)cc1 ; c1ccc(-c2cc3ccc(-c4ccc5ccc6cccnc6c5n4)cc3c3c2c(-c2ccccc2)nn3-c2ccccc2)cc1 ; c1ccc(-c2cc3ccc(-c4ccc(-c5ccc6ccc7cccnc7c6n5)cc4)cc3c3c2c(-c2ccccc2)nn3-c2ccccc2)cc1]

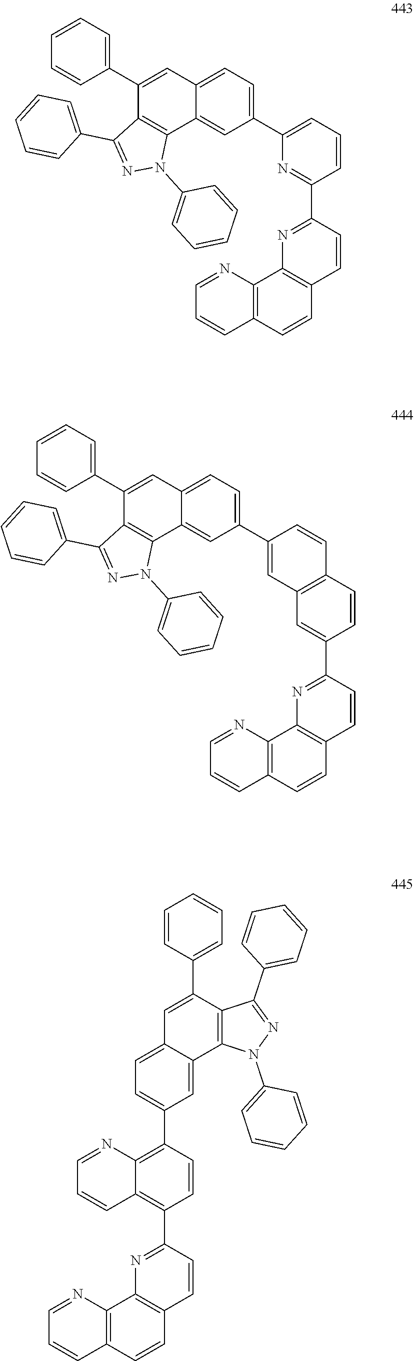 [SMILES: c1ccc(-c2cc3ccc(-c4cccc(-c5ccc6ccc7cccnc7c6n5)n4)cc3c3c2c(-c2ccccc2)nn3-c2ccccc2)cc1 ; c1ccc(-c2cc3ccc(-c4ccc5ccc(-c6ccc7ccc8cccnc8c7n6)cc5c4)cc3c3c2c(-c2ccccc2)nn3-c2ccccc2)cc1 ; c1ccc(-c2cc3ccc(-c4ccc(-c5ccc6ccc7cccnc7c6n5)c5cccnc45)cc3c3c2c(-c2ccccc2)nn3-c2ccccc2)cc1]

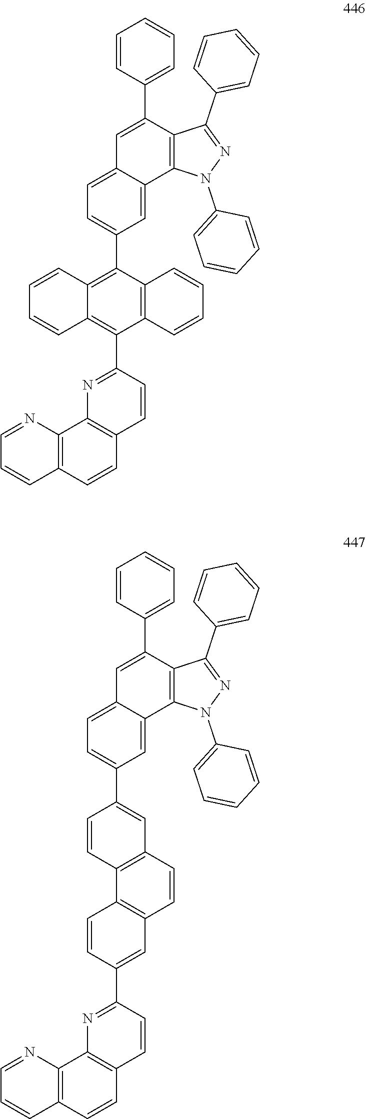 [SMILES: c1ccc(-c2cc3ccc(-c4c5ccccc5c(-c5ccc6ccc7cccnc7c6n5)c5ccccc45)cc3c3c2c(-c2ccccc2)nn3-c2ccccc2)cc1 ; c1ccc(-c2cc3ccc(-c4ccc5c(ccc6cc(-c7ccc8ccc9cccnc9c8n7)ccc65)c4)cc3c3c2c(-c2ccccc2)nn3-c2ccccc2)cc1]

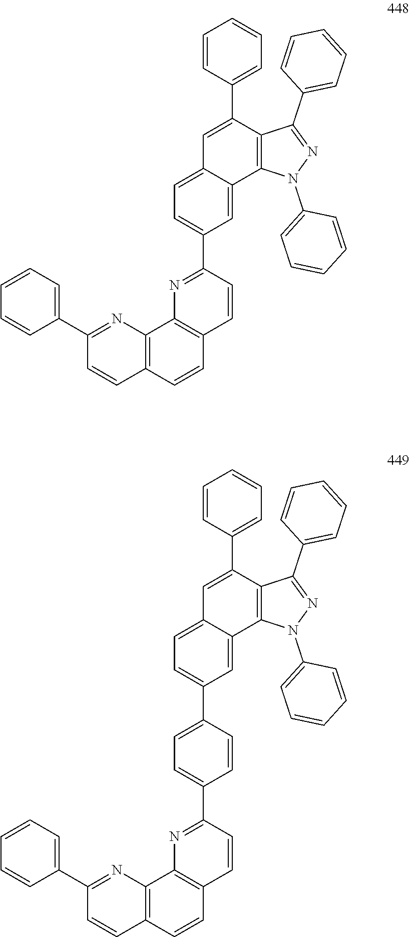 [SMILES: c1ccc(-c2ccc3ccc4ccc(-c5ccc6cc(-c7ccccc7)c7c(-c8ccccc8)nn(-c8ccccc8)c7c6c5)nc4c3n2)cc1 ; c1ccc(-c2ccc3ccc4ccc(-c5ccc(-c6ccc7cc(-c8ccccc8)c8c(-c9ccccc9)nn(-c9ccccc9)c8c7c6)cc5)nc4c3n2)cc1]

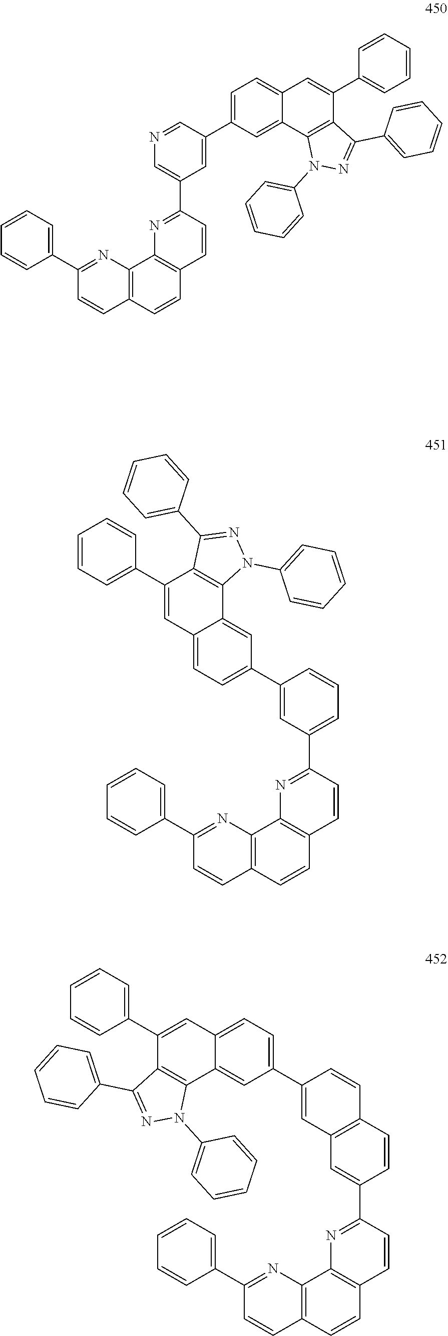 [SMILES: c1ccc(-c2ccc3ccc4ccc(-c5cncc(-c6ccc7cc(-c8ccccc8)c8c(-c9ccccc9)nn(-c9ccccc9)c8c7c6)c5)nc4c3n2)cc1 ; c1ccc(-c2ccc3ccc4ccc(-c5cccc(-c6ccc7cc(-c8ccccc8)c8c(-c9ccccc9)nn(-c9ccccc9)c8c7c6)c5)nc4c3n2)cc1 ; c1ccc(-c2ccc3ccc4ccc(-c5ccc6ccc(-c7ccc8cc(-c9ccccc9)c9c(-c%10ccccc%10)nn(-c%10ccccc%10)c9c8c7)cc6c5)nc4c3n2)cc1]

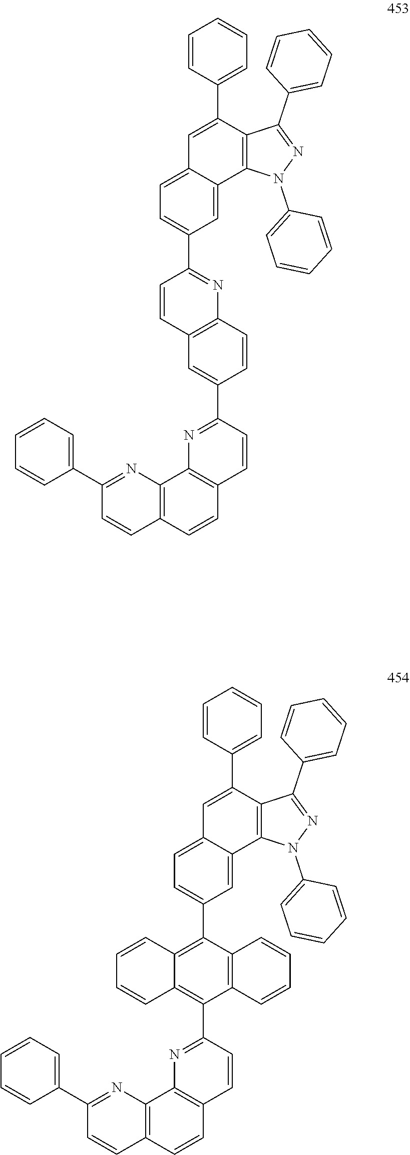 [SMILES: c1ccc(-c2ccc3ccc4ccc(-c5ccc6nc(-c7ccc8cc(-c9ccccc9)c9c(-c%10ccccc%10)nn(-c%10ccccc%10)c9c8c7)ccc6c5)nc4c3n2)cc1 ; c1ccc(-c2ccc3ccc4ccc(-c5c6ccccc6c(-c6ccc7cc(-c8ccccc8)c8c(-c9ccccc9)nn(-c9ccccc9)c8c7c6)c6ccccc56)nc4c3n2)cc1]

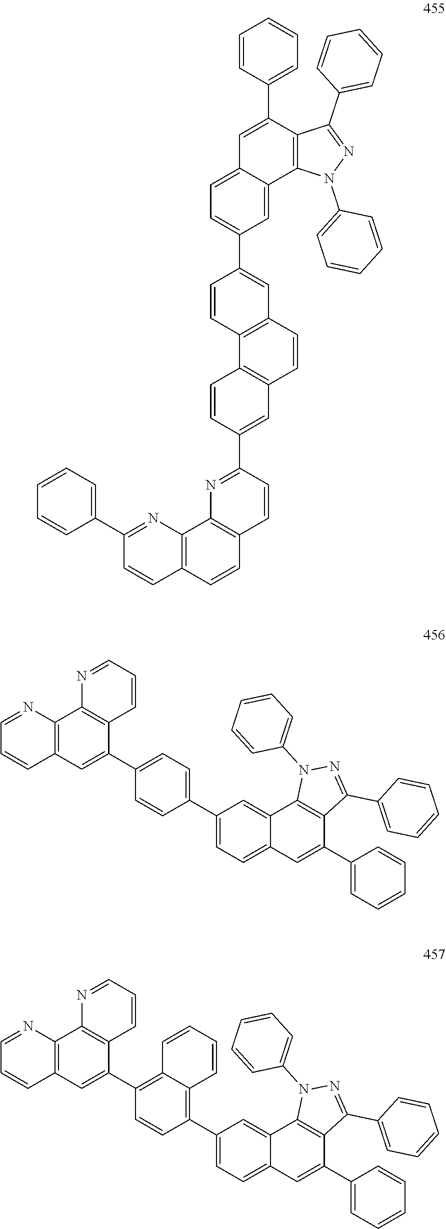 [SMILES: c1ccc(-c2ccc3ccc4ccc(-c5ccc6c(ccc7cc(-c8ccc9cc(-c%10ccccc%10)c%10c(-c%11ccccc%11)nn(-c%11ccccc%11)c%10c9c8)ccc76)c5)nc4c3n2)cc1 ; c1ccc(-c2cc3ccc(-c4ccc(-c5cc6cccnc6c6ncccc56)cc4)cc3c3c2c(-c2ccccc2)nn3-c2ccccc2)cc1 ; c1ccc(-c2cc3ccc(-c4ccc(-c5cc6cccnc6c6ncccc56)c5ccccc45)cc3c3c2c(-c2ccccc2)nn3-c2ccccc2)cc1]

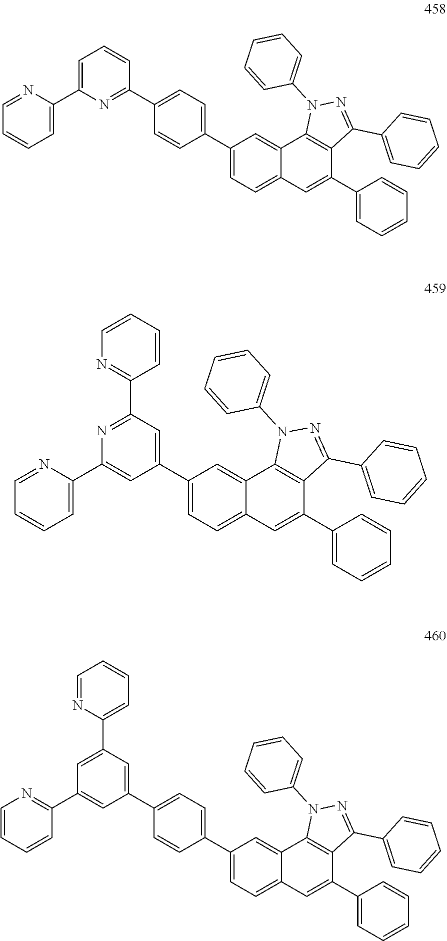 [SMILES: c1ccc(-c2cc3ccc(-c4ccc(-c5cccc(-c6ccccn6)n5)cc4)cc3c3c2c(-c2ccccc2)nn3-c2ccccc2)cc1 ; c1ccc(-c2cc3ccc(-c4cc(-c5ccccn5)nc(-c5ccccn5)c4)cc3c3c2c(-c2ccccc2)nn3-c2ccccc2)cc1 ; c1ccc(-c2cc3ccc(-c4ccc(-c5cc(-c6ccccn6)cc(-c6ccccn6)c5)cc4)cc3c3c2c(-c2ccccc2)nn3-c2ccccc2)cc1]

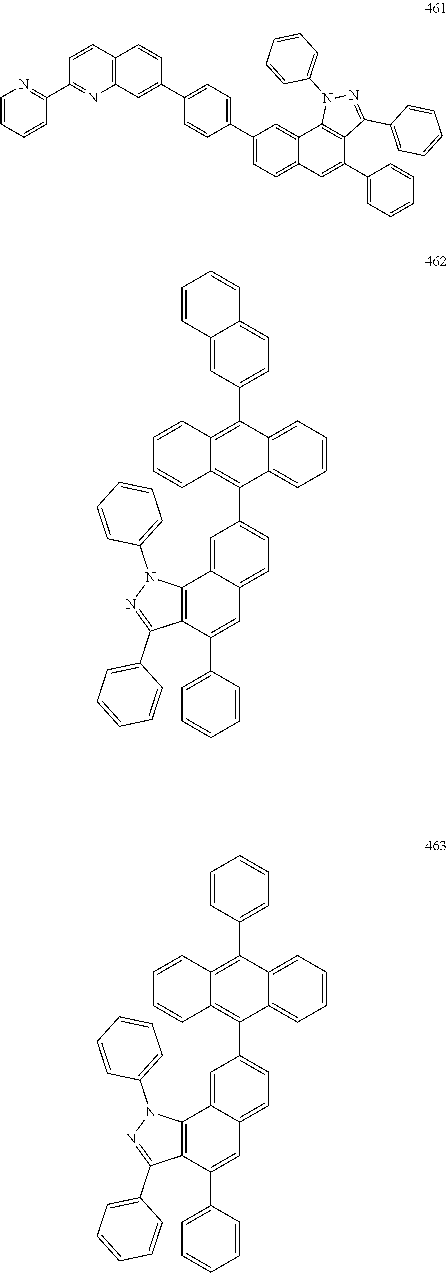 [SMILES: c1ccc(-c2cc3ccc(-c4ccc(-c5ccc6ccc(-c7ccccn7)nc6c5)cc4)cc3c3c2c(-c2ccccc2)nn3-c2ccccc2)cc1 ; c1ccc(-c2cc3ccc(-c4c5ccccc5c(-c5ccc6ccccc6c5)c5ccccc45)cc3c3c2c(-c2ccccc2)nn3-c2ccccc2)cc1 ; c1ccc(-c2c3ccccc3c(-c3ccc4cc(-c5ccccc5)c5c(-c6ccccc6)nn(-c6ccccc6)c5c4c3)c3ccccc23)cc1]

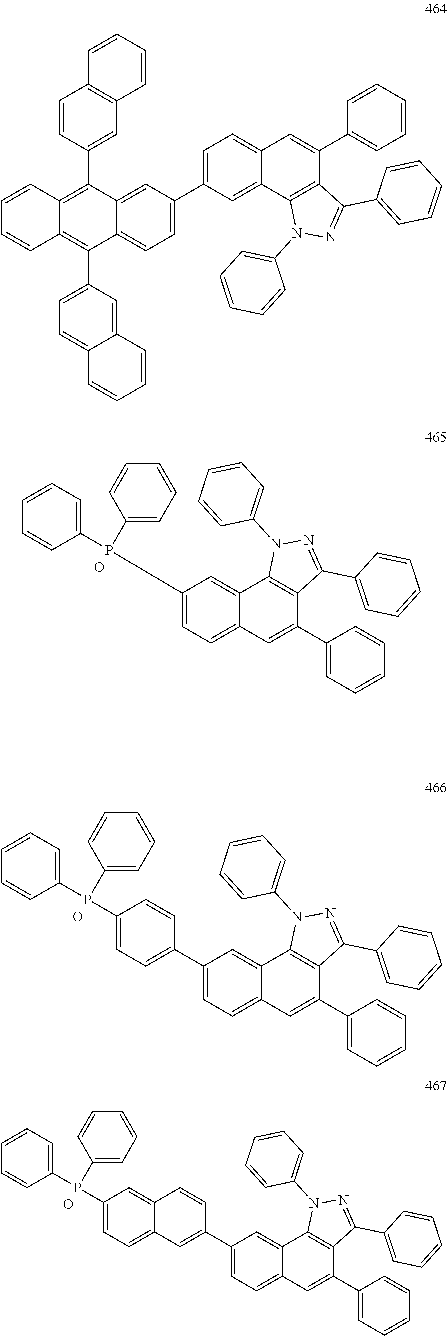

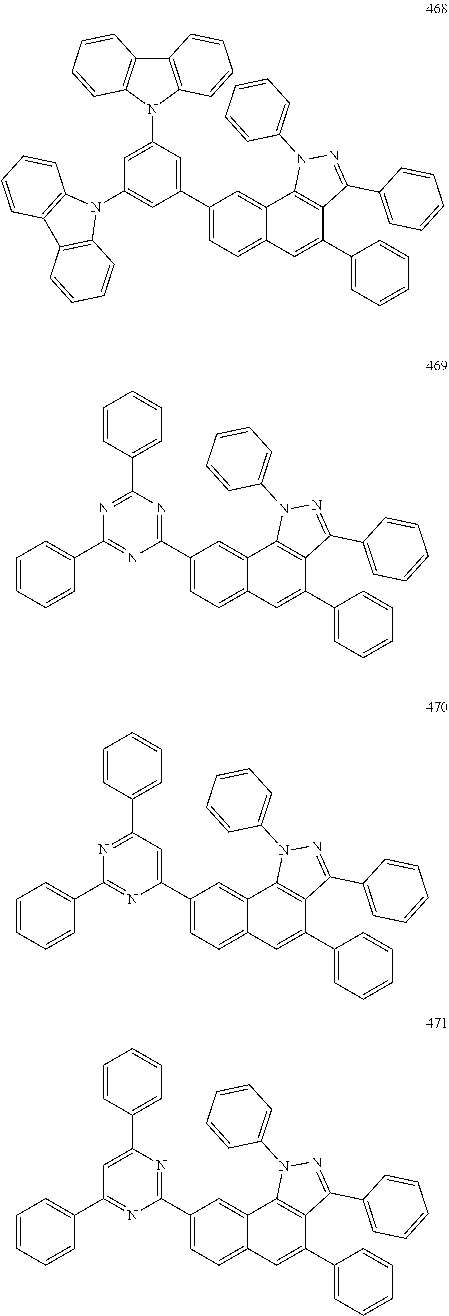 [SMILES: c1ccc(-c2cc3ccc(-c4cc(-n5c6ccccc6c6ccccc65)cc(-n5c6ccccc6c6ccccc65)c4)cc3c3c2c(-c2ccccc2)nn3-c2ccccc2)cc1 ; c1ccc(-c2nc(-c3ccccc3)nc(-c3ccc4cc(-c5ccccc5)c5c(-c6ccccc6)nn(-c6ccccc6)c5c4c3)n2)cc1 ; c1ccc(-c2cc(-c3ccc4cc(-c5ccccc5)c5c(-c6ccccc6)nn(-c6ccccc6)c5c4c3)nc(-c3ccccc3)n2)cc1 ; c1ccc(-c2cc(-c3ccccc3)nc(-c3ccc4cc(-c5ccccc5)c5c(-c6ccccc6)nn(-c6ccccc6)c5c4c3)n2)cc1]

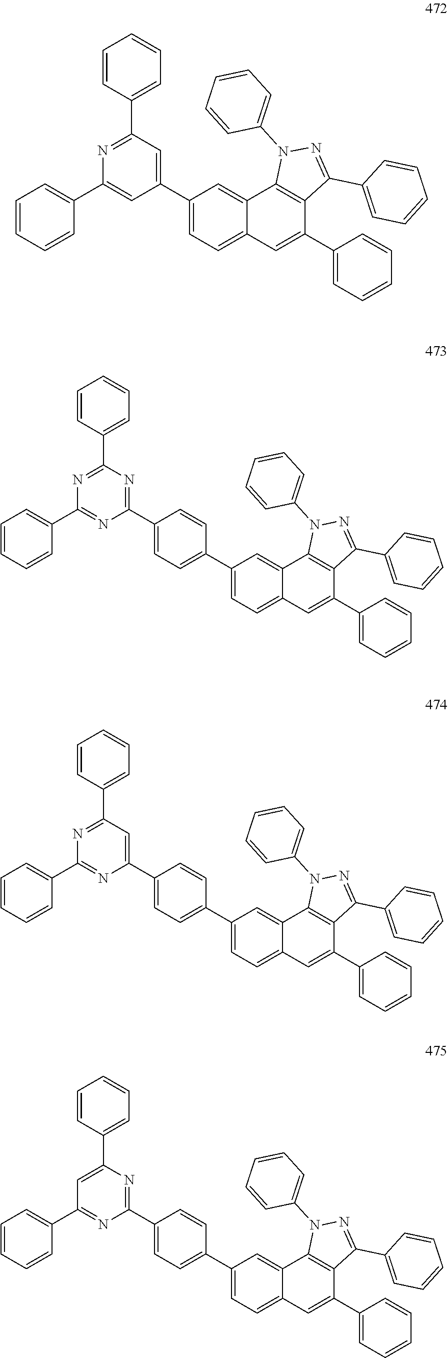 [SMILES: c1ccc(-c2cc(-c3ccc4cc(-c5ccccc5)c5c(-c6ccccc6)nn(-c6ccccc6)c5c4c3)cc(-c3ccccc3)n2)cc1 ; c1ccc(-c2nc(-c3ccccc3)nc(-c3ccc(-c4ccc5cc(-c6ccccc6)c6c(-c7ccccc7)nn(-c7ccccc7)c6c5c4)cc3)n2)cc1 ; c1ccc(-c2cc(-c3ccc(-c4ccc5cc(-c6ccccc6)c6c(-c7ccccc7)nn(-c7ccccc7)c6c5c4)cc3)nc(-c3ccccc3)n2)cc1 ; c1ccc(-c2cc(-c3ccccc3)nc(-c3ccc(-c4ccc5cc(-c6ccccc6)c6c(-c7ccccc7)nn(-c7ccccc7)c6c5c4)cc3)n2)cc1]

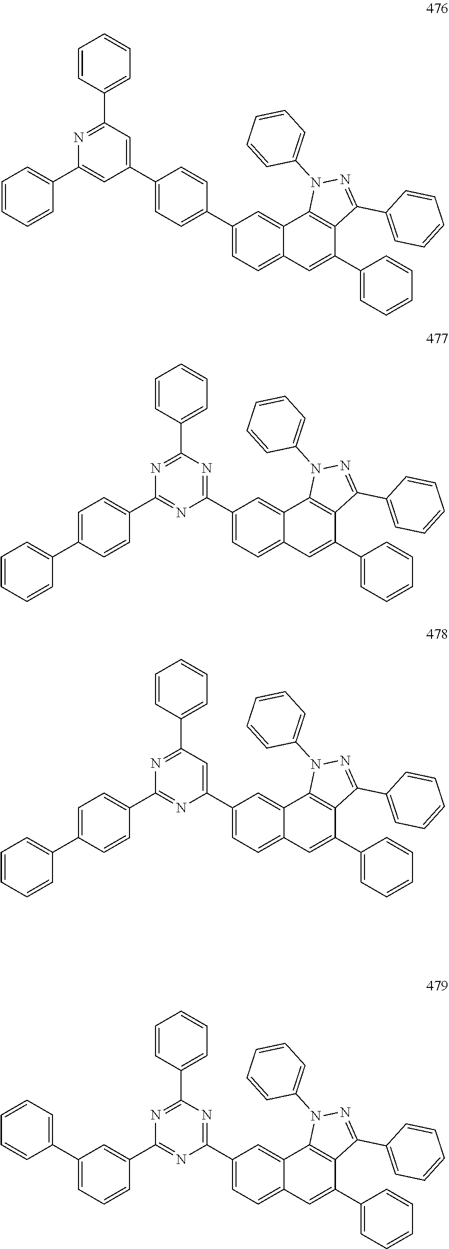 [SMILES: c1ccc(-c2cc(-c3ccc(-c4ccc5cc(-c6ccccc6)c6c(-c7ccccc7)nn(-c7ccccc7)c6c5c4)cc3)cc(-c3ccccc3)n2)cc1 ; c1ccc(-c2ccc(-c3nc(-c4ccccc4)nc(-c4ccc5cc(-c6ccccc6)c6c(-c7ccccc7)nn(-c7ccccc7)c6c5c4)n3)cc2)cc1 ; c1ccc(-c2ccc(-c3nc(-c4ccccc4)cc(-c4ccc5cc(-c6ccccc6)c6c(-c7ccccc7)nn(-c7ccccc7)c6c5c4)n3)cc2)cc1 ; c1ccc(-c2cccc(-c3nc(-c4ccccc4)nc(-c4ccc5cc(-c6ccccc6)c6c(-c7ccccc7)nn(-c7ccccc7)c6c5c4)n3)c2)cc1]

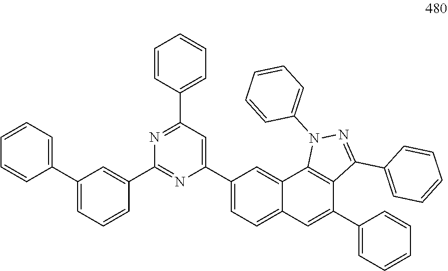 [SMILES: c1ccc(-c2cccc(-c3nc(-c4ccccc4)cc(-c4ccc5cc(-c6ccccc6)c6c(-c7ccccc7)nn(-c7ccccc7)c6c5c4)n3)c2)cc1]

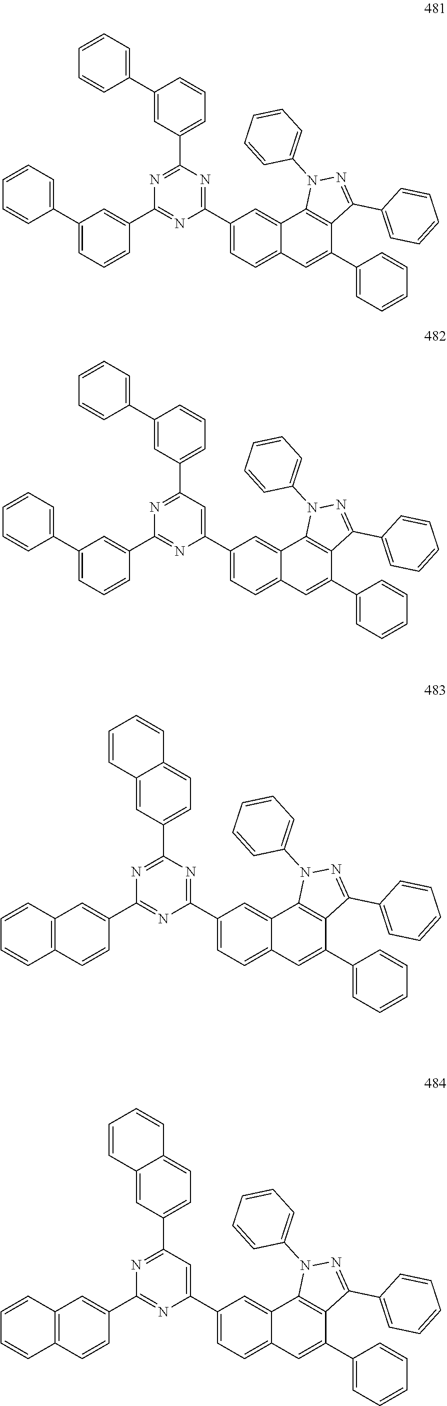

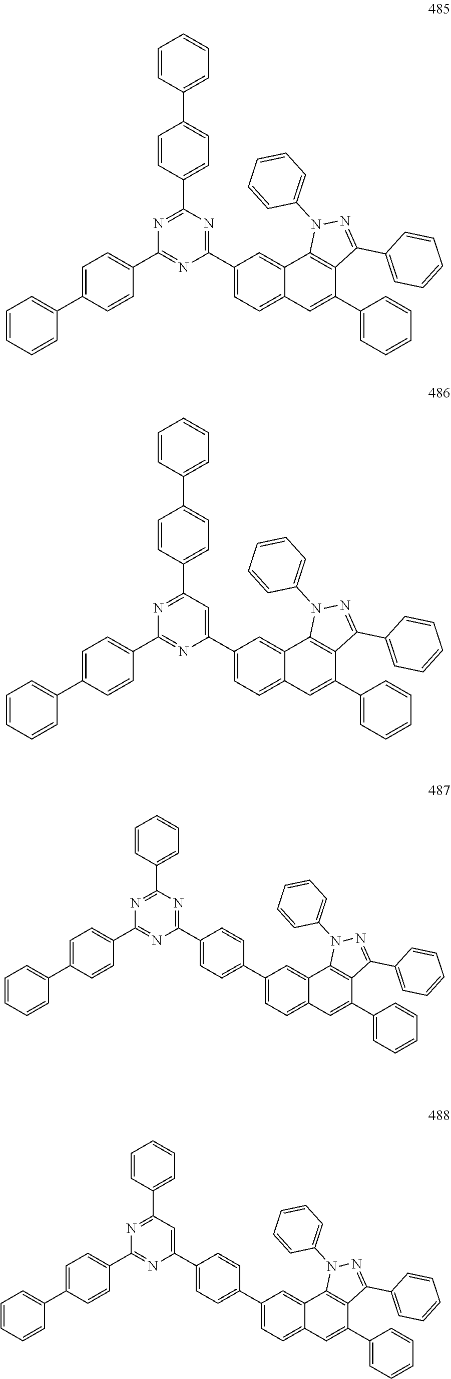

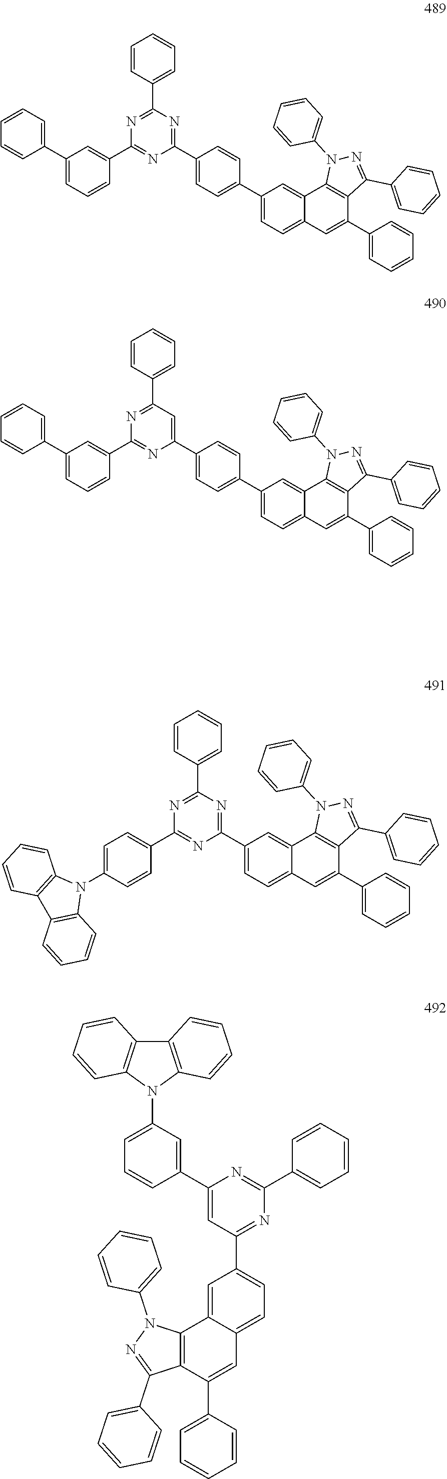

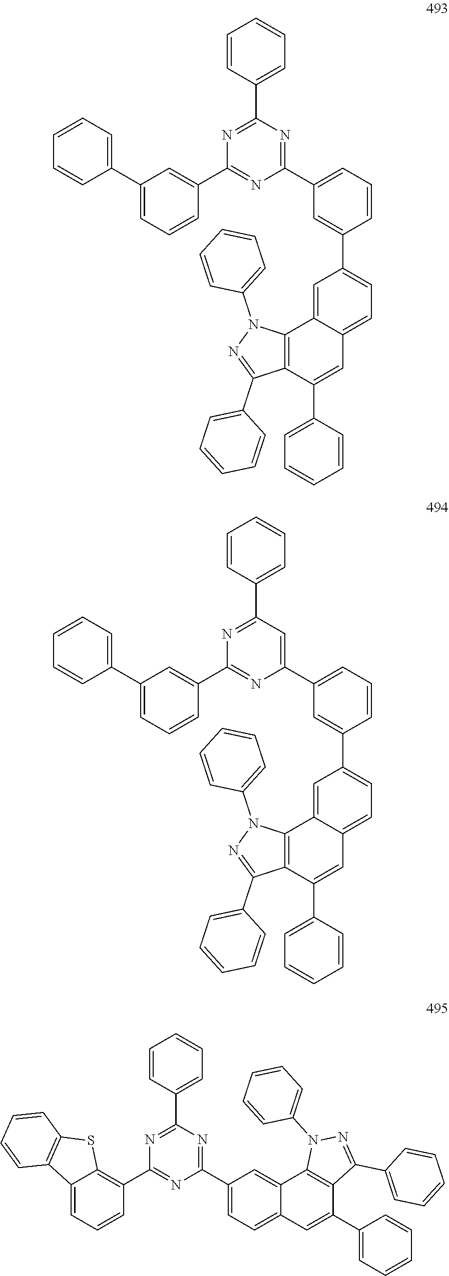

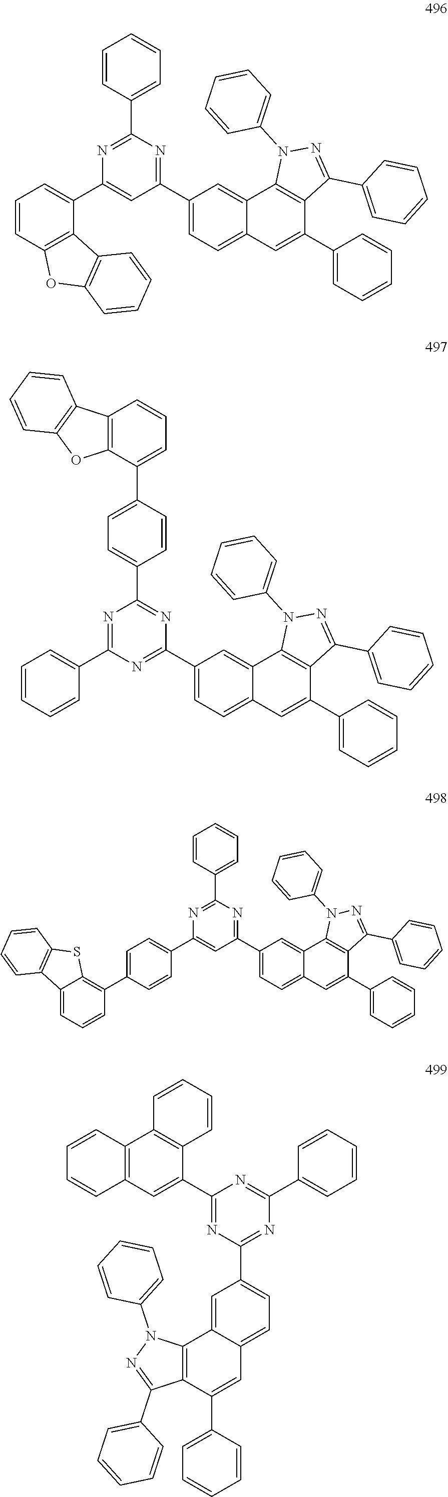

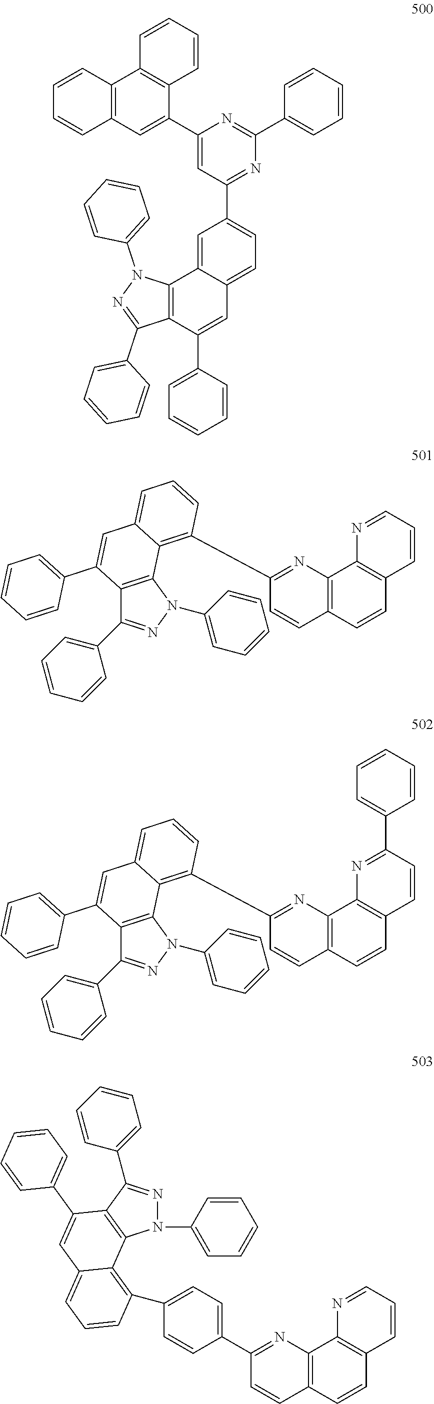

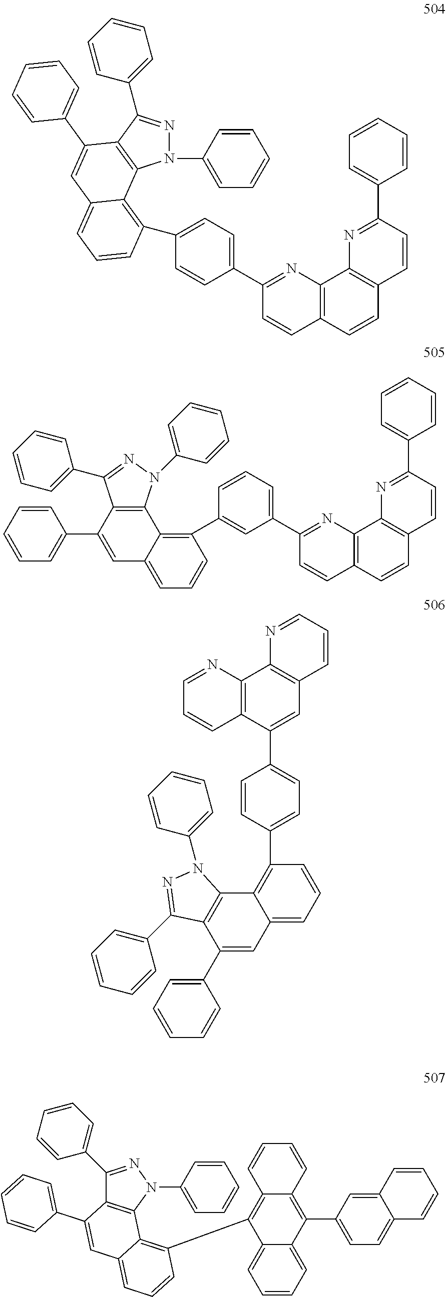

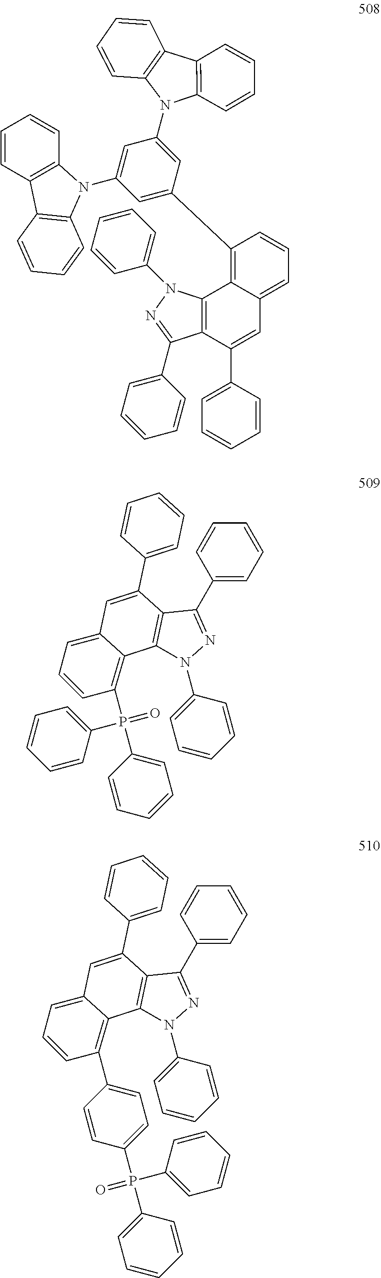

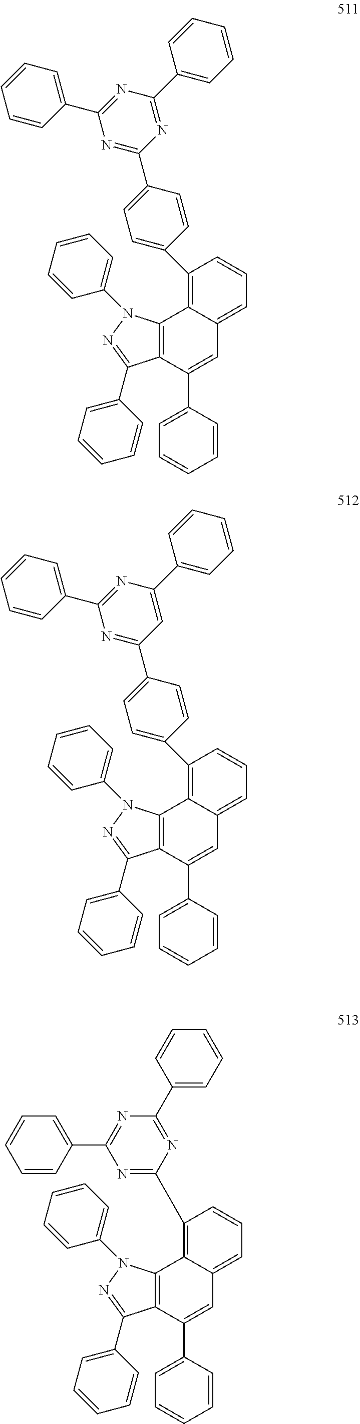

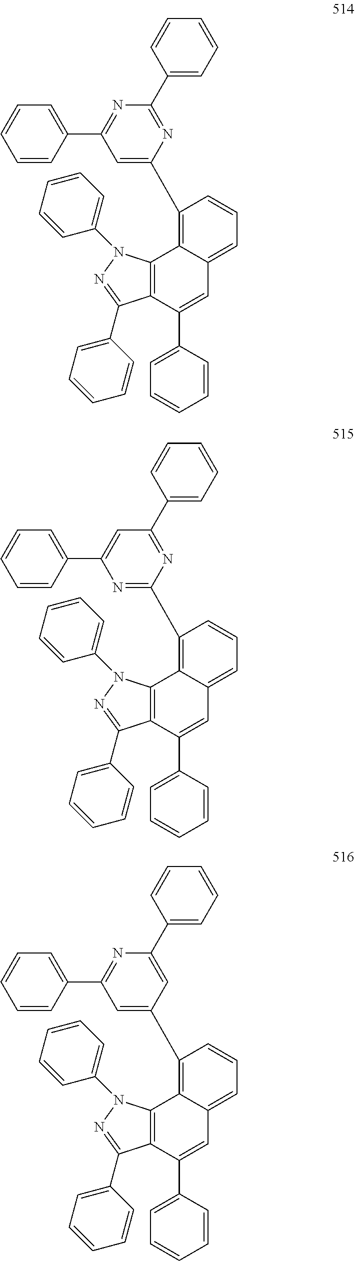

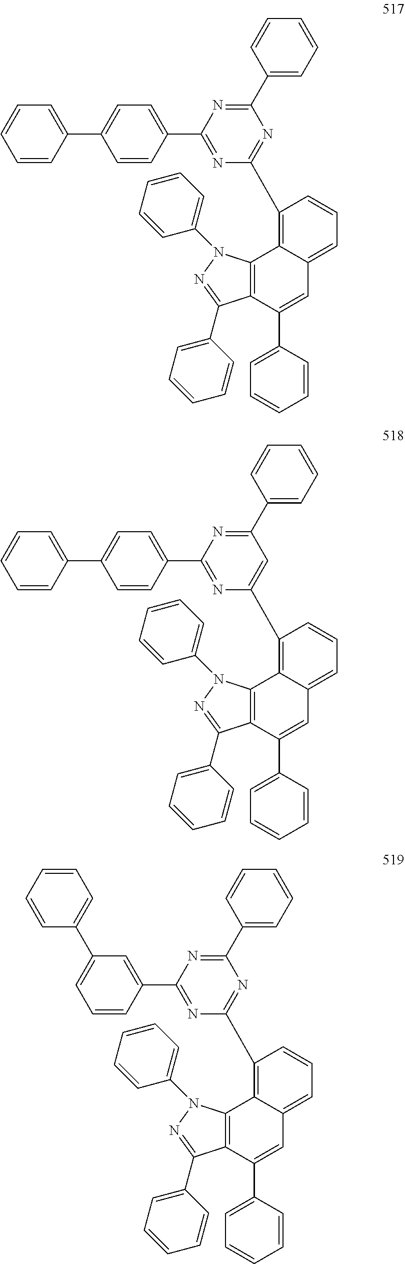

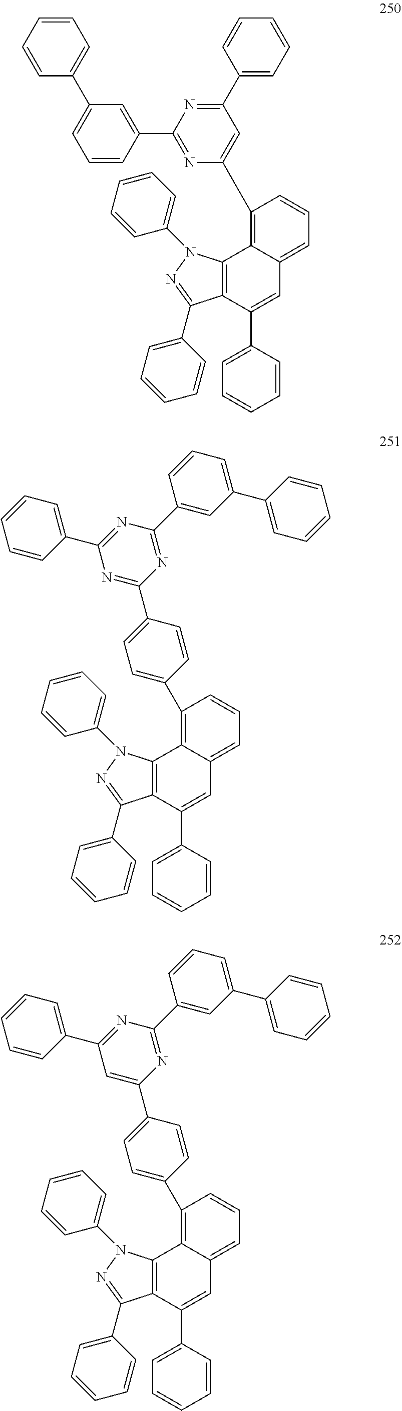

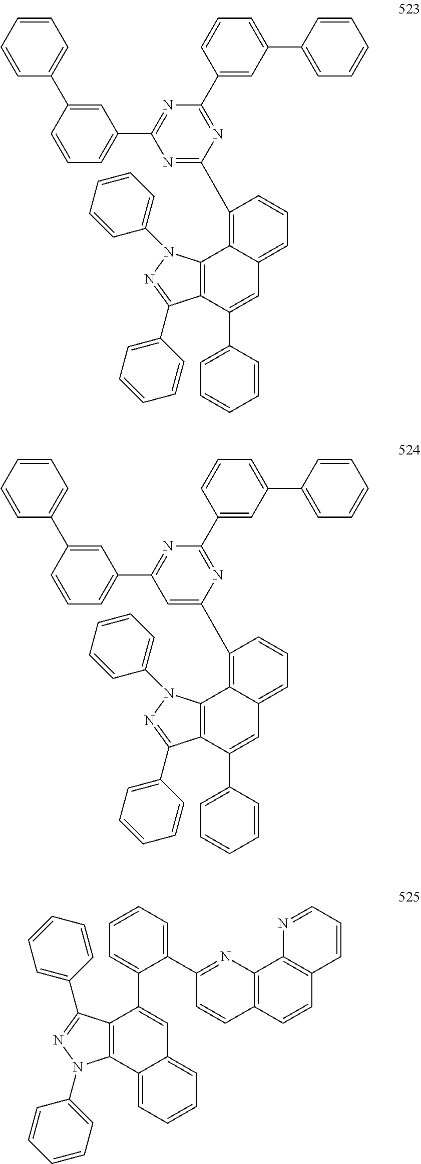 [SMILES: c1ccc(-c2cccc(-c3nc(-c4cccc(-c5ccccc5)c4)nc(-c4cccc5cc(-c6ccccc6)c6c(-c7ccccc7)nn(-c7ccccc7)c6c45)n3)c2)cc1 ; c1ccc(-c2cccc(-c3cc(-c4cccc5cc(-c6ccccc6)c6c(-c7ccccc7)nn(-c7ccccc7)c6c45)nc(-c4cccc(-c5ccccc5)c4)n3)c2)cc1 ; c1ccc(-c2nn(-c3ccccc3)c3c2c(-c2ccccc2-c2ccc4ccc5cccnc5c4n2)cc2ccccc23)cc1]

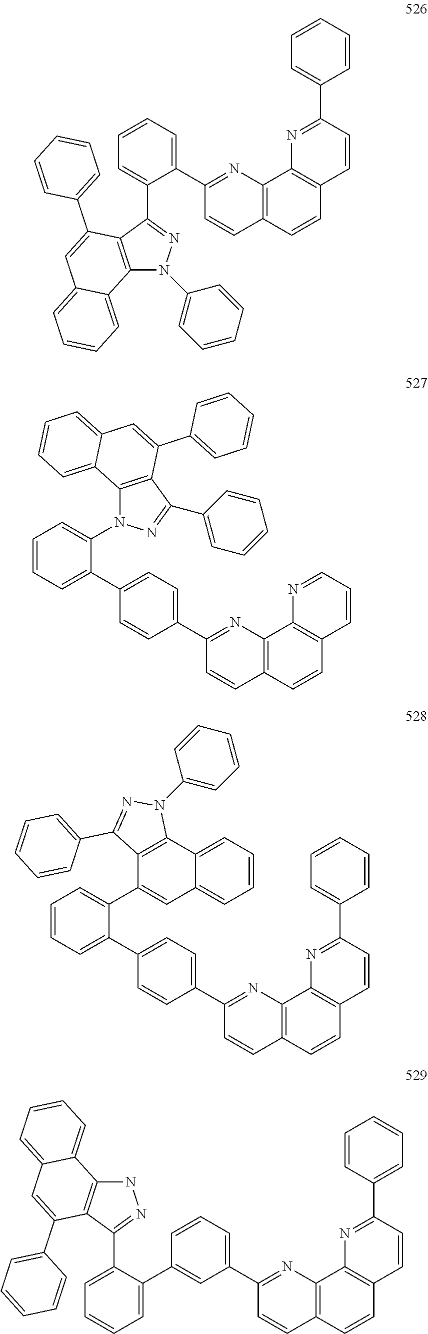

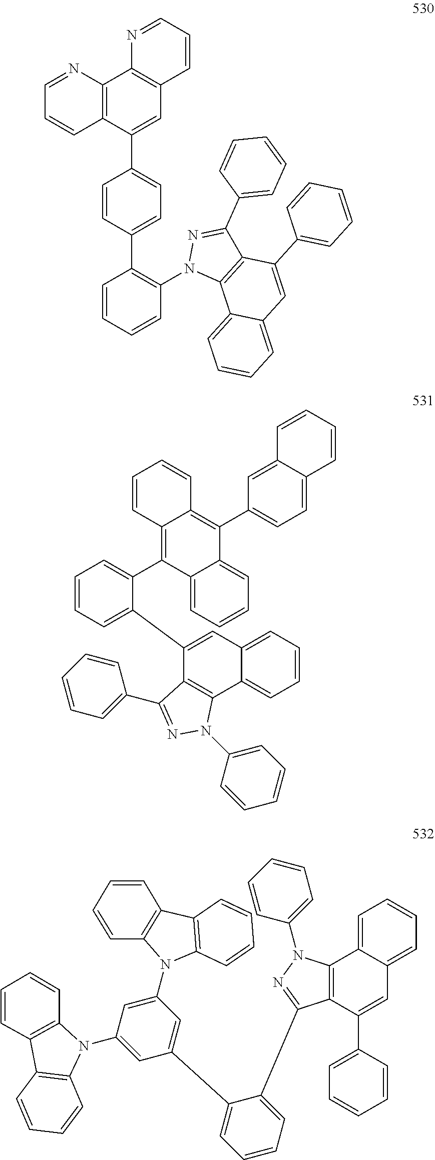 [SMILES: c1ccc(-c2cc3ccccc3c3c2c(-c2ccccc2)nn3-c2ccccc2-c2ccc(-c3cc4cccnc4c4ncccc34)cc2)cc1 ; c1ccc(-c2nn(-c3ccccc3)c3c2c(-c2ccccc2-c2c4ccccc4c(-c4ccc5ccccc5c4)c4ccccc24)cc2ccccc23)cc1 ; c1ccc(-c2cc3ccccc3c3c2c(-c2ccccc2-c2cc(-n4c5ccccc5c5ccccc54)cc(-n4c5ccccc5c5ccccc54)c2)nn3-c2ccccc2)cc1]

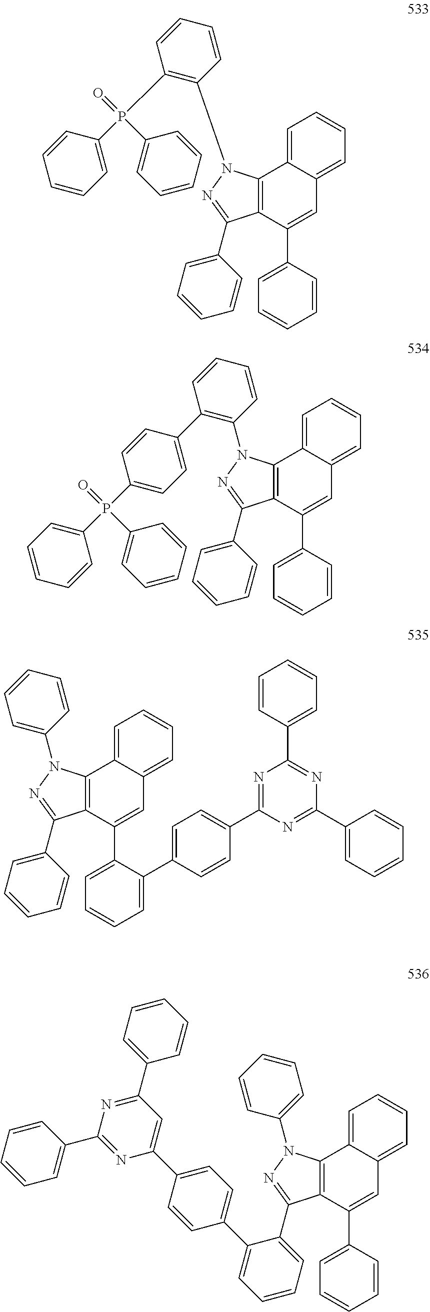

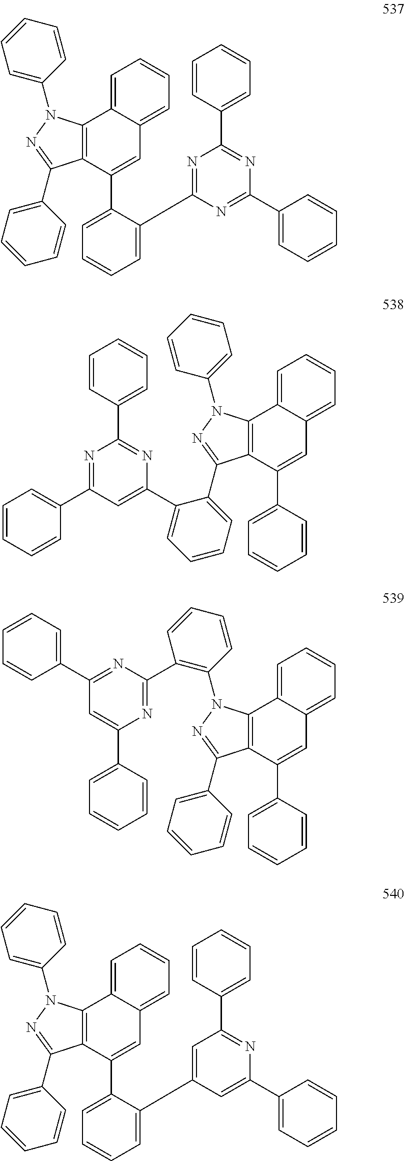

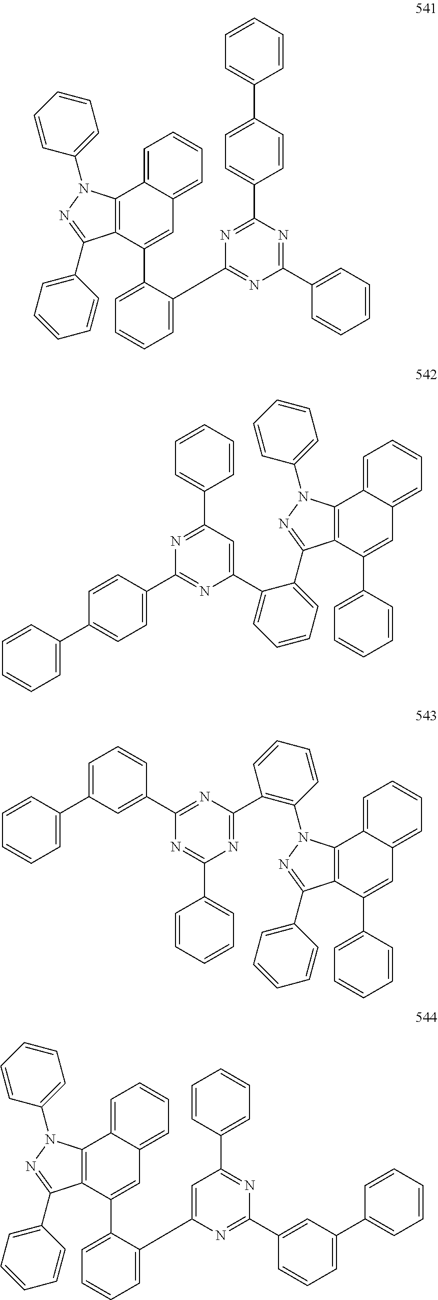 [SMILES: c1ccc(-c2ccc(-c3nc(-c4ccccc4)nc(-c4ccccc4-c4cc5ccccc5c5c4c(-c4ccccc4)nn5-c4ccccc4)n3)cc2)cc1 ; c1ccc(-c2ccc(-c3nc(-c4ccccc4)cc(-c4ccccc4-c4nn(-c5ccccc5)c5c4c(-c4ccccc4)cc4ccccc45)n3)cc2)cc1 ; c1ccc(-c2cccc(-c3nc(-c4ccccc4)nc(-c4ccccc4-n4nc(-c5ccccc5)c5c(-c6ccccc6)cc6ccccc6c54)n3)c2)cc1 ; c1ccc(-c2cccc(-c3nc(-c4ccccc4)cc(-c4ccccc4-c4cc5ccccc5c5c4c(-c4ccccc4)nn5-c4ccccc4)n3)c2)cc1]

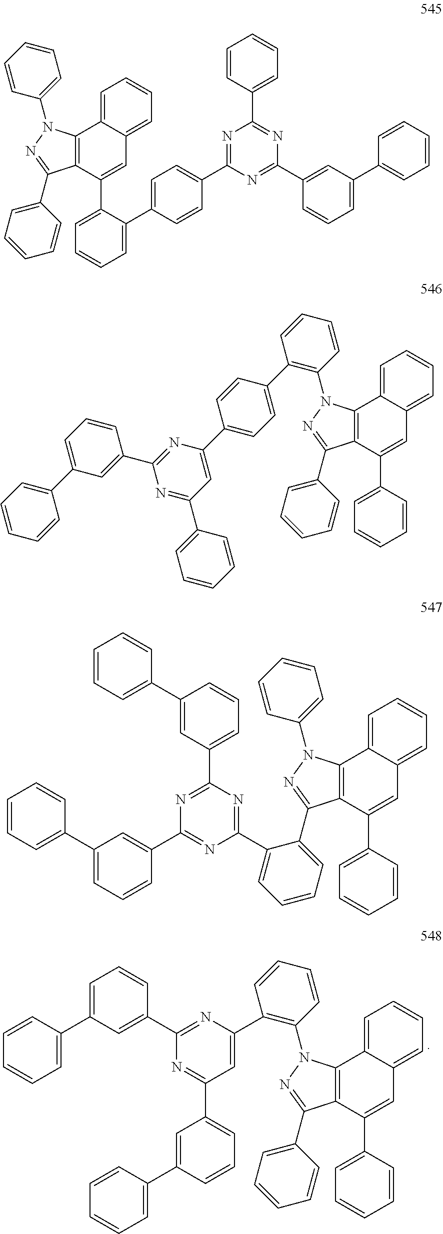 [SMILES: c1ccc(-c2cccc(-c3nc(-c4ccccc4)nc(-c4ccc(-c5ccccc5-c5cc6ccccc6c6c5c(-c5ccccc5)nn6-c5ccccc5)cc4)n3)c2)cc1 ; c1ccc(-c2cccc(-c3nc(-c4ccccc4)cc(-c4ccc(-c5ccccc5-n5nc(-c6ccccc6)c6c(-c7ccccc7)cc7ccccc7c65)cc4)n3)c2)cc1 ; c1ccc(-c2cccc(-c3nc(-c4cccc(-c5ccccc5)c4)nc(-c4ccccc4-c4nn(-c5ccccc5)c5c4c(-c4ccccc4)cc4ccccc45)n3)c2)cc1 ; c1ccc(-c2cccc(-c3cc(-c4ccccc4-n4nc(-c5ccccc5)c5c(-c6ccccc6)cc6ccccc6c54)nc(-c4cccc(-c5ccccc5)c4)n3)c2)cc1]

10. An organic light-emitting device comprising:
a first electrode;
a second electrode provided to face the first electrode; and
one or more organic layers provided between the first electrode and the second electrode, and
wherein one or more of the organic layers comprise the heterocyclic compound according to claim 1.

11. The organic light-emitting device according to claim 10, wherein the organic layer comprises an electron transport layer, wherein the electron transport layer comprises the heterocyclic compound.

12. The organic light-emitting device according to claim 10, wherein the organic layer comprises an electron injection layer or an electron transport layer, wherein the electron injection layer or the electron transport layer comprises the heterocyclic compound.

13. The organic light-emitting device according to claim 10, wherein the organic layer comprises an electron-blocking layer or a hole-blocking layer, wherein the electron-blocking layer or the hole-blocking layer comprises the heterocyclic compound.

14. The organic light-emitting device according to claim 10, wherein the organic layer comprises a first stack including one or more light-emitting layers; and a second stack including one or more light-emitting layers, and comprises one or more charge-generating layers including the heterocyclic compound between the first stack and the second stack.

15. The organic light-emitting device according to claim 10, wherein the organic layer comprises a first stack including one or more light-emitting layers; a second stack including one or more light-emitting layers; and a third stack including one or more light-emitting layers, and comprises one or more charge-generating layers each including the heterocyclic compound between the first stack and the second stack and between the second stack and the third stack.

16. The organic light-emitting device according to claim 14, wherein the charge-generating layer is an N-type charge-generating layer.

17. A composition for forming an organic layer of an organic light-emitting device, comprising the heterocyclic compound according to claim 1.

18. The composition for forming an organic layer according to claim 17, wherein the organic layer is an electron transport layer, a charge-generating layer, an electron injection layer, an electron-blocking layer, or a hole-blocking layer.

19. The organic light-emitting device according to claim 15, wherein the charge-generating layer is an N-type charge-generating layer.
